(19) United States
(12) Patent Application Publication
(10) Pub. No.: US 2009/0036430 A1
(43) Pub. Date: Feb. 5, 2009

(54) PYRIDO(3,2-D)PYRIMIDINES AND PHARMACEUTICAL COMPOSITIONS USEFUL FOR MEDICAL TREATMENT

(76) Inventors: Steven Cesar Alfons De Jonghe, Tervuren (BE); Eduard Dolusic, Wijgmaal (BE); Ling-Jie Gao, Bierbeek (BE); Piet Andre Maurits Maria Herdewijn, Rotselaar/Wezemaal (BE); Wolfgang Eugen Pfleiderer, Konstanz (DE)

Correspondence Address: CLARK & ELBING LLP, 101 FEDERAL STREET, BOSTON, MA, 02110, US

(21) Appl. No.: 12/143,652

(22) Filed: Jun. 20, 2008

Related U.S. Application Data
(63) Continuation-in-part of application No. 11/771,924, filed on Jun. 29, 2007.
Application No. 11/771,924 is a continuation-in-part of application No. PCT/EP2005/014187, filed on Dec. 29, 2005.
(60) Provisional application No. 60/693,899, filed on Jun. 24, 2005.

(30) Foreign Application Priority Data
Dec. 30, 2004 (GB) 0428475.8

(51) Int. Cl. A61K 31/551; C07D 471/02; A61K 31/519; A61K 31/496; A61P 19/02; A61P 37/06; A61P 17/00; C07D 413/14; A61K 31/5377; C07D 243/08

(52) U.S. Cl. 514/218; 544/279; 514/264.1; 514/252.16; 544/117; 514/234.2; 540/575

(57) ABSTRACT

This invention relates to substituted pyrido(3,2-d)pyrimidine derivatives, their pharmaceutically acceptable salts, N-oxides, solvates, pro-drugs and enantiomers, possessing unexpectedly desirable pharmaceutical properties, in particular which are highly active immunosuppressive agents, and as such are useful in the treatment in transplant rejection and/or in the treatment of certain inflammatory diseases. These derivatives are also useful in preventing or treating cardiovascular disorders, disorders of the central nervous system, TNF-α related disorders, viral diseases (including hepatitis C), erectile dysfunction and cell proliferative disorders.

### CROSS REFERENCE TO RELATED APPLICATIONS

This application is a continuation-in-part of U.S. application Ser. No. 11/771,924, filed Jun. 29, 2007, which is a continuation-in-part of International Application No. PCT/EP2005/014187, filed Dec. 29, 2005, which claims the benefit of U.K. patent application No. 0428475.8, filed Dec. 30, 2004, and U.S. provisional application No. 60/693,899, filed Jun. 24, 2005, the disclosures of which are incorporated by reference in their entirety.

The present invention relates to a class of novel pyrido(3,2-d)pyrimidine derivatives and a method for their preparation, as well as to pharmaceutical compositions comprising one or more of said pyrido(3,2-d)pyrimidine derivatives and one or more pharmaceutically acceptable excipients. The present invention further relates to the use of said novel pyrido(3,2-d)pyrimidine derivatives as biologically active ingredients, more specifically as medicaments for the treatment of disorders and pathologic conditions such as, but not limited to, immune and auto-immune disorders, organ and cells transplant rejections, cell proliferative disorders, cardiovascular disorders, disorders of the central nervous system and viral diseases.

### BACKGROUND OF THE INVENTION

A huge number of pyrido(3,2-d)pyrimidine derivatives is already known in the art. For instance pyrido(3,2-d)pyrimidine derivatives with various substituents on positions 2, 4 and 6 (using the standard atom numbering for the pyrido(3,2-d)pyrimidine moiety) are known with biological activities such as competitive inhibition of pteroylglutamic acid, inhibition of thrombocyte aggregation and adhesiveness, antineoplastic activity, inhibition of dihydrofolate reductase and thymidylate synthase, e.g. from U.S. Pat. No. 2,924,599, U.S. Pat. No. 3,939,268, U.S. Pat. No. 4,460,591, U.S. Pat. No. 5,167,963 and U.S. Pat. No. 5,508,281.

Pyrido(3,2-d)pyrimidine derivatives with various substituents on positions 2, 4, 6 and 7 (using the standard atom numbering for the pyrido(3,2-d)pyrimidine moiety) are also known e.g. from U.S. Pat. No. 5,521,190, U.S. patent application publication No. 2002/0049207, U.S. patent application publication No. 2003/0186987, U.S. patent application publication No. 2003/0199526, U.S. patent application publication No. 2004/0039000, U.S. patent application publication No. 2004/0106616, U.S. Pat. No. 6,713,484, U.S. Pat. No. 6,730,682 and U.S. Pat. No. 6,723,726. Some of them show activities as antiviral agents, anti-cancer agents, EGF inhibitors, inhibitors of GSK-3 protein kinases and the like.

U.S. Pat. No. 5,654,307 discloses pyrido(3,2-d)pyrimidine derivatives which are substituted on position 4 with monoarylamino or monobenzylamino, and on positions 6 and 7 with substituents each independently selected from the group consisting of lower alkyl, amino, lower alkoxy, mono- or dialkylamino, halogen and hydroxy. WO 01/083456 discloses pyrido(3,2-d)pyrimidine derivatives which are substituted on position 4 with morpholinyl and on position 2 with hydroxyphenyl or morpholinoethoxyphenyl, having PI3K and cancer inhibiting activity. U.S. Pat. No. 6,476,031 generically discloses substituted quinazoline derivatives, including (in reaction scheme 5) a series of pyrido(3,2-d)pyrimidine derivatives which are substituted on position 4 with hydroxy, chloro or an aryl, heteroaryl (including pyridyl, pyrimidyl, indolyl, benzimidazolyl, benzotriazolyl, isoquinolyl, quinolyl, benzothiazolyl, benzofuranyl, thienyl, furyl, pyrrolyi, thiazolyl, oxazolyl, imidazolyl), cycloaliphatic or cycloheteroaliphatic group being optionally spaced from the pyrido(3,2-d)pyrimidine ring by a linker such as NH. WO 02/22602 and WO 02/22607 disclose pyrazole and triazole compounds, including 2-(1-trifluoromethylphenyl)-4-fluorobenzopyrazoiyl-pyrido(3,2-d)pyrimidine and 2-(1-trifluoromethylphenyl)-4-methyltriazolyl-pyrido(3,2-d)pyrimidine being useful as protein kinase inhibitors. WO 03/062209 discloses pyrido(3,2-d)pyrimidine derivatives which are substituted on position 7 with aryl or heteoaryl and on position 4 with monoarylamino or monoheteroarylamino and which may further be substituted on positions 2 and/or 6, being useful as capsaicin receptor modulators. However none of these documents teaches or suggests pyrido(3,2-d)pyrimidine derivatives having the substitution pattern disclosed by the present invention.

However there is a continuous need in the art for specific and highly therapeutically active compounds, such as, but not limited to, drugs for treating immune and autoimmune disorders, organ and cells transplant rejections, cell proliferative disorders, cardiovascular disorders, disorders of the central nervous system, allergic conditions and viral diseases. In particular, there is a need in the art to provide immunosuppressive compounds, antineoplastic drugs and anti-viral drugs which are active in a minor dose in order to replace existing drugs having significant side effects and to decrease treatment costs.

Currently used immunosuppressive drugs include antiproliferative agents, such as methotrexate (a 2,4-diaminopyrido(3,2-d)pyrimidine derivative disclosed by U.S. Pat. No. 2,512,572), azathioprine, and cyclophosphamide. Since these drugs affect mitosis and cell division, they have severe toxic effects on normal cells with high turn-over rate such as bone marrow cells and the gastrointestinal tract lining. Accordingly, marrow depression and liver damage are common side effects of these antiproliferative drugs.

Anti-inflammatory compounds used to induce immunosuppression include adrenocortical steroids such as dexamethasone and prednisolone. The common side effects observed with the use of these compounds are frequent infections, abnormal metabolism, hypertension, and diabetes.

Other immunosuppressive compounds currently used to inhibit lymphocyte activation and subsequent proliferation include cyclosporine, tacrolimus and rapamycin. Cyclosporine and its relatives are among the most commonly used immunosuppressant drugs. Cyclosporine is typically used for preventing or treating organ rejection in kidney, liver, heart, pancreas, bone marrow, and heart-lung transplants, as well as for the treatment of autoimmune and inflammatory diseases such as Crohn's disease, aplastic anemia, multiple-sclerosis, myasthenia gravis, uveitis, biliary cirrhosis, etc. However, cyclosporines suffer from a small therapeutic dose window and severe toxic effects including nephrotoxicity, hepatotoxicity, hypertension, hirsutism, cancer, and neurotoxicity.

Additionally, monoclonal antibodies with immunosuppressant properties, such as OKT3, have been used to prevent and/or treat graft rejection. Introduction of such monoclonal antibodies into a patient, as with many biological materials, induces several side-effects, such as dyspnea. Within the context of many life-threatening diseases, organ transplantation is considered a standard treatment and, in many cases, the only alternative to death. The immune response to foreign cell surface antigens on the graft, encoded by the major histo-compatibility complex (hereinafter referred as MHC) and present on all cells, generally precludes successful transplantation of tissues and organs unless the transplant tissues come from a compatible donor and the normal immune response is suppressed. Other than identical twins, the best compatibility and thus, long term rates of engraftment, are achieved using MHC identical sibling donors or MHC identical unrelated cadaver donors. However, such ideal matches are difficult to achieve. Further, with the increasing need of donor organs an increasing shortage of transplanted organs currently exists. Accordingly, xenotransplantation has emerged as an area of intensive study, but faces many hurdles with regard to rejection within the recipient organism.

The host response to an organ allograft involves a complex series of cellular interactions among T and B lymphocytes as well as macrophages or dendritic cells that recognize and are activated by foreign antigen. Co-stimulatory factors, primarily cytokines, and specific cell-cell interactions, provided by activated accessory cells such as macrophages or dendritic cells are essential for T-cell proliferation. These macrophages and dendritic cells either directly adhere to T-cells through specific adhesion proteins or secrete cytokines that stimulate T-cells, such as IL-12 and IL-15. Accessory cell-derived co-stimulatory signals stimulate activation of interleukin-2 (IL-2) gene transcription and expression of high affinity IL-2 receptors in T-cells. IL-2 is secreted by T lymphocytes upon antigen stimulation and is required for normal immune responsiveness. IL-2 stimulates lymphoid cells to proliferate and differentiate by binding to IL-2 specific cell surface receptors (IL-2R). IL-2 also initiates helper T-cell activation of cytotoxic T-cells and stimulates secretion of interferon-γ which in turn activates cytodestructive properties of macrophages. Furthermore, IFN-γ and IL-4 are also important activators of MHC class II expression in the transplanted organ, thereby further expanding the rejection cascade by enhancing the immunogenicity of the grafted organ. The current model of a T-cell mediated response suggests that T-cells are primed in the T-cell zone of secondary lymphoid organs, primarily by dendritic cells. The initial interaction requires cell to cell contact between antigen-loaded MHC molecules on antigen-presenting cells (hereinafter referred as APC) and the T-cell receptor/CD3 complex on T-cells. Engagement of the TCR/CD3 complex induces CD154 expression predominantly on CD4 T-cells that in turn activate the APC through CD40 engagement, leading to improved antigen presentation. This is caused partly by upregulation of CD80 and CD86 expression on the APC, both of which are ligands for the important CD28 co-stimulatory molecule on T-cells. However, engagement of CD40 also leads to prolonged surface expression of MHC-antigen complexes, expression of ligands for 4-1BB and OX-40 (potent co-stimulatory molecules expressed on activated T-cells). Furthermore, CD40 engagement leads to secretion of various cytokines (e.g., IL-12, IL-15, TNF-α, IL-1, IL-6, and IL-8) and chemokines, all of which have important effects on both APC and T-cell activation and maturation. Similar mechanisms are involved in the development of auto-immune disease, such as type I diabetes. In humans and non-obese diabetic mice, insulin-dependent diabetes mellitus results from a spontaneous T-cell dependent auto-immune destruction of insulin-producing pancreatic .beta. cells that intensifies with age. The process is preceded by infiltration of the islets with mononuclear cells (insulitis), primarily composed of T lymphocytes. A delicate balance between auto-aggressive T-cells and suppressor-type immune phenomena determines whether expression of auto-immunity is limited to insulitis or not. Therapeutic strategies that target T-cells have been successful in preventing further progress of the auto-immune disease. These include neonatal thymectomy, administration of cyclosporine, and infusion of anti-pan T-cell, anti-CD4, or anti-CD25 (IL-2R) monoclonal antibodies. The aim of all rejection prevention and auto-immunity reversal strategies is to suppress the patient's immune reactivity to the antigenic tissue or agent, with a minimum of morbidity and mortality. Accordingly, a number of drugs are currently being used or investigated for their immunosuppressive properties. As discussed above, the most commonly used immunosuppressant is cyclosporine, which however has numerous side effects. Accordingly, in view of the relatively few choices for agents effective at immunosuppression with low toxicity profiles and manageable side effects, there exists a need in the art for identification of alternative immunosuppressive agents and for agents acting as complement to calcineurin inhibition.

The metastasis of cancer cells represents the primary source of clinical morbidity and mortality in the large majority of solid tumors. Metastasis of cancer cells may result from the entry of tumor cells into either lymphatic or blood vessels. Invasion of lymphatic vessels results in metastasis to regional draining lymph nodes. From the lymph nodes, melanoma cells for example tend to metastasize to the lung, liver, and brain. For several solid tumors, including melanoma, the absence or the presence of lymph nodes metastasis is the best predictor of patient survival. Presently, to our knowledge, no treatment is capable of preventing or significantly reducing metastasis. Hence, there is a need in the art for compounds having such anti-metastasis effect for a suitable treatment of cancer patients.

Septic shock is a major cause of death in intensive care units (about 150,000 estimated deaths annually in the United States of America, despite treatment with intravenous antibiotics and supportive care) for which very little effective treatment is available at present. Patients with severe sepsis often experience failures of various systems in the body, including the circulatory system, as well as kidney failure, bleeding and clotting. Lipopolysaccharide (hereinafter referred as LPS) is the primary mediator of Gramm-negative sepsis, the most common form of sepsis, by inducing the production of a whole array of macrophage-derived cytokines (such as TNF-α; interleukins such as IL-1, IL-6, IL-12; interferon-gamma (hereinafter referred IFN-γ), etc.). These cytokines may induce other cells (e.g. T cells, NK cells) to make cytokines as well (e.g. IFN-γ). In addition, other macrophage products (e.g. nitric oxide, hereinafter referred as NO) may also play a role in the pathogenesis of toxic shock. These substances (e.g. NO) may be induced directly due to microbial interactions or indirectly through the action of proinflammatory cytokines. LPS binds to a serum protein known as LPB and the LPS-LPB complex thus formed is recognized by the CD14 toll-like receptor 4 (hereinafter referred as Tlr 4) complex on mononuclear phagocytes. Tlr4 is a signal transducing unit, the activation of which results in the release of mediators such as TNF-α, IL-1α, IL-1β and IL-6. These cytokines are important for the pathogenesis of shock. Their administration produces the clinical symptoms of septic shock and their blockade partially protects against LPS-induced lethal shock.

Current therapeutic strategies for the treatment of septic shock are directed against LPS (e.g. antibodies against LPS or LBP-34-23) or against the cytokines induced by LPS (e.g. TNF antibodies) or against the receptor for LPS (e.a. CD14). Unfortunately the initial clinical data of these approaches are very disappointing and illustrate the redundancy of receptors and mediators involved in the pathogenesis of toxic shock. For instance flagellin seems to be another toxin that plays a role in Gramm-negative Salmonella shock syndrome and that cannot be prevented or treated by therapeutic strategies directed specifically at LPS.

Clinical trials in humans with TNF-α blocking antibodies (such as the IL-1 receptor antagonist or PAF receptor antagonists) have been unsuccessful yet, as have been approaches to down regulate inflammation (e.g. using prednisolone) or to block endotoxins. These products must be administered very early after the onset of the disease, which is in most cases not possible.

The only drug currently approved by health authorities for the treatment of adult patients with the most serious forms of sepsis, including septic shock, is a genetically engineered version of a naturally occurring human protein, Activated Protein C, known as Xigris® or drotecogin-alpha which shows only moderate efficacy. Furthermore, because Activated Protein C interferes with blood clotting, the most serious side effect associated with Xigris® is bleeding, including bleeding that causes stroke. Thus Xigris® is contra-indicated for patients who have active internal bleeding, or who are more likely to bleed because of certain medical conditions including recent strokes, recent head or spinal surgery or severe head trauma. Because treatment with Xigris® comes with potentially serious risks, the benefits and risks of treatment with Xigris® must be carefully weighed for each individual patient.

Therefore there is a strong need in the art for new medications, either alone or in combination with the currently suggested treatments, for treating the most serious forms of life-threatening illnesses caused by severe infection, such as septic shock.

TNF-α is generally considered to be the key mediator in the mammalian response to bacterial infection. It is a strong pro-inflammatory agent that will affect the function of almost any organ system, either directly or by inducing the formation of other cytokines like IL-1 or prostaglandines. TNF-α is also a potent anti-tumor agent. If administered in small quantities to humans, it causes fever, headache, anorexia, myalgia, hypotension, capillary leak syndrome, increased rates of lipolysis and skeletal muscle protein degradation (including cachexia). Its use in cancer treatment is therefore very much limited by its severe side effects.

TNF-α, a pleiotropic cytokine produced mainly by activated macro-phages, exerts an in vitro cytotoxic action against transformed cells and in vivo anti-tumor activities in animal models. However, despite the fact that TNF-α is used in cancer patients especially to treat melanoma and sarcoma, the major problem hampering its use is toxicity. Indeed, TNF-α induces shock-like symptoms such as bowel swelling and damage, liver cell necrosis, enhanced release of inflammatory cytokines such as IL-1 or IL-6, and hypo-tension probably due to the release of inducers of vessels dilatation such nitric oxide and other proinflammatory cytokines. Cardiovascular toxicity is usually dose-limiting. Hypotension can be severe with systolic blood pressure below 60 mm Hg. Respiratory compromise is common after treatment with TNF-α and may require mechanical ventilation. Upper as well as lower digestive tract symptoms are also common in this type of treatment. Nausea and vomiting can be distressing and in some cases dose-limiting. Watery diarrhea is frequently observed. Neurological sequelae of treatment with TNF-α can also occur.

Hence, compounds that inhibit the toxic effects of TNF-α but that do not inhibit TNF-α anti-tumor effect are highly desirable for the treatment of cancer patients. Presently, several clinical trials involving TNF-α are being developed for the cancer of organs such as liver, lung, kidney and pancreas, which are based on a procedure including the steps of organ isolation, injection of TNF-α into the isolated organ, and reperfusion of the treated organ. However, even for isolated organ perfusion, some TNF-α usually escapes to the general blood circulation and leads to the mortality of about 10% of the patients thus treated. Many patients treated by this procedure also require intensive care unit rescue to cope with the toxic side-effects of such TNF-α treatment.

Combined treatment of TNF-α with alkylating drugs in an isolated organ perfusion model has received considerable attention. TNF-α is currently successfully used in isolated limb perfusion of human cancer patients and, in combination with melphalan and interferon-gamma, against melanoma, sarcomas and carcinomas.

The gastrointestinal mucosa is very sensitive to chemotherapeutic drugs. Mucositis caused by chemotherapy usually begins rapidly after initiation of the treatment with inflammation and ulceration of the gastrointestinal tract and leading to diarrhea. Severe, potentially life-threatening, diarrhea may require interruption of the chemotheraputic treatment and subsequent dose reduction of the therapeutic agent. The oral cavity is often the place of severe side effects from cancer therapy that adversely affects the quality of life of the patient and its ability to tolerate the therapy. These side effects can be caused by radiotherapy as well as chemotherapy. A relationship between both serum and mucosal levels of TNF-α and IL-1 correlates with nonhematologic toxicities, including mucositis.

Radiation injuries occurring e.g. after a single high-dose irradiation include apoptosis as well as radiation necrosis. Even normal tissues protected by shielding during irradiation may be considerably damaged. It was found in experimental animal models that the radiation injuries after a single high-dose irradiation typically used for the treatment of various malignant tumors consist of radiation necrosis and apoptosis, which were correlated with the expression of TNF-α and TGF-β1.

Irradiation may induce graft-versus-host disease (hereinafter referred as GVHD) in cancer patients. This disease may occur especially in patients receiving allogeneic bone marrow transplantation as a treatment for cancers such as leukemia or lymphoma and can lead to the death of about 25% of the relevant patients. Before bone marrow transplantation, leukaemia patients for example receive either total body or total lymphoid irradiation to suppress their immune system. However, such irradiation induces not only necrosis but also the release of proinflammatory cytokines mainly TNF-α, IL-1 and IL-6 which in turn induce direct host tissues inflammation and activation of donor cells against host antigens leading to GVHD.

Cisplatin is an effective chemotherapeutic agent used in the treatment of a wide variety of both pediatric and adult malignancies, including testicular, germ cell, head and neck (cervical), bladder and lung cancer. Dose-dependent and cumulative nephrotoxicity is the major side effect of cisplatin, sometimes requiring a reduction in dose or discontinuation of the treatment. Other side effects of cisplatin include kidney damage, loss of fertility, harmful effect on a developing baby, temporary drop in bone marrow function causing drop in white blood cell count, anaemia, drop in platelets causing bleeding, loss of appetite, numbness or tingling in limbs, loss of taste, allergic reactions, and hearing disorders (difficulty in hearing some high-pitched sounds, experiencing ringing in the ears). Blurred vision may also be a side effect with high doses of cisplatin. It was shown that TNF-α is a key element in a network of proinflammatory chemokines and cytokines activated in the kidney by cisplatin. Blockade of TNF-α action would prevent the activation of this cytokine network and would provide protection against cisplatin nephrotoxicity. Hence, compounds that inhibit the toxic effects of cisplatin but that do not inhibit cisplatin anti-tumor effects are highly desirable for the treatment of cancer patients.

A surplus of TNF-α also causes a dramatic change of endothelial cells. In particular, TNF-α is an important mediator of skeletal muscle degeneration associated with cachexia, a debilitating syndrome characterized by extreme weight loss and whole-body wasting. Cachexia is usually a secondary condition whereby there is excessive tissue catabolism in combination with deficient anabolism. It is frequently seen in patients afflicted with chronic diseases such as cancer, cardiopulmonary diseases, aging, malabsortive disorders, excessive physical stress, eating disorders and acquired immuno-deficiency syndrome (AIDS). Some authors consider that the elevated TNF-α values found in at least 50% of cancer patients in the active stage of the disease can result in cachexia. TNF-α levels in clinically healthy adults, as well as in adult cancer patients, are well documented, for instance by Nenova et al. in Archives of Hellenic Medicine (2000) 17:619-621. Serum TNF-α concentrations in healthy children as well as in children with malignancies are documented for instance by Saarinen et al. in Cancer Research (1990) 50:592-595. A very significant proportion of cancer mortalities result from cachexia rather than from tumor burden. Chronic wasting disease (cachexia) may result when excessive cellular damage results in the release of substances (TNF-α, collagenase, hyaluronidase) that further catabolize the so-called healthy tissue resulting in an inability to assimilate nutrients required for anabolic restructuring of associated tissue.

Infants infected with human immunodeficiency virus type 1 (HIV-1) show growth retardation and severe weight loss that can lead to death. The overproduction of certain cytokines has been implicated as a possible cause for this. For instance, according to Rautonen et al. in AIDS (1991) 5:1319-1325, serum IL-6 concentrations are elevated and associated with elevated TNF-α concentrations in children with HIV infection. Swapan et al. in Journal of Virology (2002) 76:11710-11714 have shown that reduction of TNF-α levels by either anti-TNF-α antibodies or human chorionic gonadotropin inhibits the expression of HIV-1 proteins and prevents cachexia and death.

Very few drugs have been suggest at present for the treatment of cachexia. Some high-dose progestins like megestrol acetate, an agent used for the treatment of metastatic breast cancer, and medroxyprogesterone acetate were shown in randomized clinical trials to provide a statistically significant advantage as regards improved appetite and body weight gain. Hence, compounds that stimulate appetite and body weight gain without inhibiting the anti-tumor effect or anti-viral effect of co-administered drugs are highly desirable for the treatment of cachexia. More specifically, there is a need in the art for treating cachexia by the administration of compounds that reduce TNF-α levels in the serum of humans.

TNF-α is also suspected to play a role, through a possible dual action in the hematopoietic environment, in the development of hematologic malignancies such as idiopathic myelodysplastic syndromes occurring most often in elderly people but also occasionally in children, these syndromes being currently regarded as the early phase of acute leukemia.

Phosphodiesterases are a family of enzymes that hydrolyse cyclic nucleotide intracellular second messengers to their non-cyclic form. Cyclic 3′,5′-adenosine monophosphate (cAMP) modulates a variety of cellular and physiologic functions in mammals, such as, cell division, endocrine function, and the immune response. The level of cAMP is controlled by a class of enzymes called phosphodiesterases, which enzymatically deactivate cAMP. There are eleven types of phosphodiesterases which are categorized according to their function and the type of cell from which they are isolated. For instance, high-affinity phosphodiesterase (PDE-3) is isolated from human platelet cells and modulates platelet aggregation. Another type of phosphodiesterase (PDE-4) is found in various tissues but is the predominant form in human leukocytes; this enzyme modulates leukocyte activation and function associated with the immune response and inflammation. Both of these phosphodiesterases implement their control by modulating the cellular level of cAMP in their respective cells. Thus, inhibition of phosphodiesterases provides a method of modulating any cellular and bodily function that is controlled by cAMP. Compounds that are non-specific phosphodiesterase inhibitors, i.e. that inhibit all or multiple types of phosphodiesterases, are known. However, since cAMP is involved in so many functions throughout the body, a non-specific phosphodiesterase inhibitor has the potential to alter all functions modulated by cAMP, thus non-specific phospho-diesterase inhibitors are of limited value because of their numerous side-effects. Phosphodiesterase-4 (hereinafter referred as PDE-4) are cAMP-specific and are the major cAMP metabolising enzymes found in inflammatory and immune cells. Thus, molecules inhibiting PDE-4 lead to an elevation of cAMP levels within inflammatory and immune cells, thus having a potential immunomodulating effect on the activation of such cells which can lead to a decreased secretion of inflammatory and immunologically important molecules such as cytokines. TNF-α is an example of such an important inflammatory cytokine. Inhibition of PDE-4 using small molecules may be expected to inhibit the production of this cytokine by inflammatory cells such as monocytes and macrophages. Preparation of Human Lymphocyte Phospho-diesterase-4, as well as Human cAMP Phosphodiesterase assays have been described for instance in U.S. Pat. No. 5,264,437. Such a biological activity is important from a therapeutic point of view since excessive inflammatory cytokine production has been associated with a number of inflammatory and immunological diseases including for example, rheumatoid arthritis, rheumatoid spondylitis asthma, Crohn's disease, inflammatory bowel disease, osteoarthritis, reperfusion injury, sepsis and septic shock, chronic obstructive pulmonary disease, graft versus host reactions and allograft rejections.

The World Health Organization estimates that world-wide 170 million people (3% of the world's population) are chronically infected with HCV. These chronic carriers are at risk of developing cirrhosis and/or liver cancer. In studies with a 10 to 20 year follow-up, cirrhosis developed in 20-30% of the patients, 1-5% of whom may develop liver cancer during the next then years. The only treatment option available today is the use of interferon a-2 (or its pegylated from) either alone or combined with ribavirin. However, sustained response to such treatment is only observed in about 40% of the patients, and treatment is associated with serious adverse effects. There is thus an urgent need in the art for potent and selective inhibitors of HCV replication in order to treat patients infected with HCV. However, investigation of specific inhibitors of HCV replication has been hampered by the fact that it is highly difficult to efficiently propagate HCV in cell culture. Since HCV and pestiviruses belong to the same virus family and share many similarities (such as, but not limited to, organisation of the genome, analogous gene products and replication cycle), pestiviruses may be adopted as a model virus and surrogate for HCV. For example the Bovine Viral Diarrhea Virus (BVDV) is closely related to hepatitis C virus (HCV) and may be used as a surrogate virus in drug development for HCV infection.

There is a strong need in the art to improve, or to provide alternatives to, the existing prophylactic or therapeutic solutions to all the aforesaid diseases. In particular there is still a need in the art for providing alternative synthetic molecules having significant TNF-α activity and/or PDE-4 activity and/or HCV replication inhibiting activity. Meeting these various needs in the art constitutes the main goal of the present invention.

### SUMMARY OF THE INVENTION

The present invention is based on the unexpected finding that certain combinations of substituents on positions 2, 4, 6 and/or 7 (using the standard atom numbering for the pyrido(3,2-d)pyrimidine moiety) which are not suggested by the prior art are however able to meet one or more of the needs recited herein above, in particular have significant TNF-α activity and/or PDE-4 activity and/or HCV replication inhibiting activity.

Based on this finding the present invention relates, in a first embodiment, to a class of pyrido(3,2-d)pyrimidine derivatives represented by the structural formula (I):

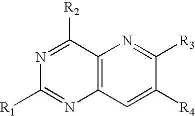

wherein:
R₁ is selected from the group consisting of hydrogen, halogen, cyano, carboxylic acid, acyl, thioacyl, alkoxycarbonyl, acyloxy, carbonate, carbamate, C₁₋₇ alkyl, aryl, amino, acetamido, N-protected amino, (mono- or di) C₁₋₇ alkylamino, (mono- or di) arylamino, (mono- or di) C₃₋₁₀ cycloalkylamino, (mono- or di) hydroxy C₁₋₇ alkylamino, (mono- or di) C₁₋₄ alkyl-arylamino, mercapto C₁₋₇ alkyl, C₁₋₇ alkyloxy, and groups of the formula R₆—NR₇R₁₂, wherein R₆ is a bond or C₁₋₃ alkylene, wherein R₇ and R₁₂ are independently selected from the group consisting of hydrogen, C₁₋₇ alkyl, C₂₋₇ alkenyl, C₂₋₇ alkynyl, aryl, arylalkyl, C₃₋₁₀ cycloalkyl and heteroaryl, or wherein R₇ and R₁₂ together form a heterocycle,
R₂ is selected from the group consisting of (mono- or di-) C₁₋₁₂ alkylamino; monoarylamino; diarylamino; (mono- or di-) C₃₋₁₀ cycloalkylamino; (mono- or di-) hydroxyC₁₋₇ alkylamino; (mono- or di-) C₁₋₄ alkylarylamino; (mono- or di-) arylC₁₋₄ alkylamino; morpholinyl; mercapto C₁₋₇ alkyl; C₁₋₇ alkoxy, homopiperazinyl and piperazinyl, wherein said homopiperazinyl or piperazinyl is optionally N-substituted with a substituent R₅ selected from the group consisting of formyl, acyl, thioacyl, amide, thioamide, sulfonyl, sulfinyl, carboxylate, thiocarboxylate, amino-substituted acyl, alkoxyalkyl, C₃₋₁₀ cycloalkyl-alkyl, C₃₋₁₀ cycloalkyl, dialkylaminoalkyl, heterocyclic-substituted alkyl, acyl-substituted alkyl, thioacyl-substituted alkyl, amido-substituted alkyl, thioamido-substituted alkyl, carboxylato-substituted alkyl, thiocarboxylato-substituted alkyl, (amino-substituted acyl)alkyl, heterocyclic, carboxylic acid ester, w-cyanoalkyl, w-carboxylic ester-alkyl, halo C₁₋₇ alkyl, C₂₋₇ alkenyl, C₂₋₇ alkynyl, arylalkenyl, aryloxyalkyl, arylalkyl and aryl, wherein the aryl moiety of each of said arylalkenyl, aryloxyalkyl, arylalkyl and aryl radicals is optionally substituted with one or more substituents independently selected from the group consisting of halogen, C₁₋₇ alkyl, C₂₋₇ alkenyl, C₂₋₇ alkynyl, halo C₁₋₇ alkyl, nitro, hydroxyl, sulfhydryl, amino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thio-heterocyclic, arylalkylthio, heterocyclic-substituted alkylthio, formyl, carbamoyl, thiocarbamoyl, ureido, thioureido, sulfonamido, hydroxylamino, alkoxy-amino, mercaptoamino, thioalkylamino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazino, alkylhydrazino and phenylhydrazino;
R₃ and R₄ are independently selected from the group consisting of hydrogen halogen, heteroaryl and aryl groups, wherein said heteroaryl or aryl groups are optionally substituted with one or more substituents selected from the group consisting of halogen, C₁₋₇ alkyl, C₂₋₇ alkenyl, C₂₋₇ alkynyl, halo C₁₋₇ alkyl, nitro, hydroxyl, sulfhydryl, amino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thio-heterocyclic, arylalkylthio, heterocyclic-substituted alkylthio, formyl, carbamoyl, thiocarbamoyl, ureido, thioureido, sulfonamido, hydroxylamino, alkoxy-amino, mercaptoamino, thioalkylamino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazino, alkylhydrazino and phenylhydrazino, provided that R₃ and R₄ are not both hydrogen, and further provided that R₄ is hydrogen when R₂ is monoarylamino, or a pharmaceutical acceptable addition salt thereof or a stereoisomer thereof or a N-oxide thereof or a solvate thereof.

Within the above defined class of compounds, a preferred group is one wherein R₁ is not hydrogen, i.e. position 2 of the pyrido(3,2-d)pyrimidine moiety is substituted. Another preferred group of compounds is one wherein R₁ is amino or N-protected amino such as, but not limited to, acetamido. Another preferred group of compounds is one wherein R₁ is amino or N-protected amino, and further wherein R₃ is a substituted aryl group. Another preferred group of compounds is one wherein R₁ is amino or N-protected amino, wherein R₃ is a substituted aryl group and further wherein R₄ is hydrogen.

In a second embodiment, the present invention relates to certain groups of tri-substituted pyrido(3,2-d)pyrimidines which are useful as intermediates for making some of the pyrido(3,2-d)pyrimidine derivatives represented by the structural formula (I), in particular:
a group of 2-amino-4-hydroxy-6-R₃-substituted pyrido(3,2-d)pyrimidines and 2,4-diamino-6-R₃-substituted pyrido(3,2-d)pyrimidines wherein R₃ is as defined in the structural formula (I) but R₃ is not hydrogen;
a group of 2-N-protected-amino-4-hydroxy-6-R₃-substituted pyrido(3,2-d)pyrimidines, 2-N-protected-amino-4-chloro-6-R₃-substituted pyrido(3,2-d)pyrimidines and 2-N-protected-amino-4-triazolyl-6-R₃-substituted pyrido(3,2-d)pyrimidines wherein R₃ is as defined in the general formula (I) but R₃ is not hydrogen, and wherein N-protected-amino may be, but is not limited to, acetamido and pivalamido;
a group of 2-R₁-substituted-4-hydroxy-6-R₃-substituted pyrido(3,2-d)pyrimidines, 2-R₁-substituted-4-chloro-6-R₃-substituted pyrido(3,2-d)pyrimidines and 2-R₁-substituted-4-triazolyl-6-R₃-substituted pyrido(3,2-d)pyrimidines wherein R₁ and R₃ are as defined in the structural formula (I) but are not hydrogen;
a group of 2,4-dihydroxy-6-R₃-substituted pyrido(3,2-d)pyrimidines and 2,4-dichloro-6-R₃-substituted pyrido(3,2-d)pyrimidines wherein R₃ is as defined in the structural formula (I) but R₃ is not hydrogen;
a group of 2-chloro-4-R₂-substituted-6-R₃-substituted pyrido(3,2-d)pyrimidines wherein R₂ and R₃ are as defined in the structural formula (I) but are not hydrogen;
a group of 2-amino-4-hydroxy-7-R-substituted pyrido(3,2-d)pyrimidines and 2,4-diamino-7-R₄-substituted pyrido(3,2-d)pyrimidines wherein R₄ is as defined in the structural formula (I) but R₄ is not hydrogen;
a group of 2-N-protected-amino-4-hydroxy-7-R₄-substituted pyrido(3,2-d)pyrimidines, 2-N-protected-amino-4-chloro-7-R-substituted pyrido(3,2-d)pyrimidines and 2-N-protected-amino-4-triazolyl-7-R₄-substituted pyrido(3,2-d)pyrimidines wherein R₄ is as defined in the structural formula (I) but R₄ is not hydrogen, and wherein N-protected-amino may be, but is not limited to, acetamido and pivalamido;
a group of 2-R₁-substituted-4-hydroxy-7-R₄-substituted pyrido(3,2-d)pyrimidines, 2-R₁-substituted-4-chloro-7-R₄-substituted pyrido(3,2-d)pyrimidines and 2-R₁-substituted-4-triazolyl-7-R₄-substituted pyrido(3,2-d)pyrimidines wherein R₁ and R₄ are as defined in the structural formula (I) but are not hydrogen;
a group of 2,4-dihydroxy-7-R₄-substituted pyrido(3,2-d)pyrimidines and 2,4-dichloro-7-R₄-substituted pyrido(3,2-d)pyrimidines wherein R₄ is as defined in the structural formula (I) but R₄ is not hydrogen; and
a group of 2-chloro-4-R₂-substituted-7-R₄-substituted pyrido(3,2-d)pyrimidines wherein R₂ and R₄ are as defined in the structural formula (I) but are not hydrogen.

In a third embodiment, the present invention relates to the unexpected finding that at least one desirable biological property is present in the said group of novel compounds such as, but not limited to:
the ability to decrease the proliferation of lymphocytes,
the ability to decrease T-cell activation,
the ability to decrease B-cell or monocytes or macrophages activation,
the ability to inhibit the release of certain cytokines,
the ability to inhibit human TNF-α production,
the ability to inhibit phosphodiesterase-4 activity, and
the ability to inhibit hepatitis C virus (hereinafter referred as HCV) replication.
   As a consequence, the invention relates to pharmaceutical compositions comprising one or more pharmaceutically acceptable carriers and, as an active principle, at least one pyrido(3,2-d)pyrimidine derivative represented by the structural formula (I) and/or a pharmaceutically acceptable addition salt thereof and/or a stereoisomer thereof and/or a N-oxide thereof and/or a solvate thereof.

As a result of their one or more biological properties mentioned hereinabove, compounds represented by the structural formula (I) are highly active immunosuppressive agents, or antineoplastic agents, or anti-HCV agents which, together with one or more pharmaceutically acceptable carriers, may be formulated into pharmaceutical compositions for the prevention or treatment of pathologic conditions such as, but not limited to, immune and autoimmune disorders, organ and cells transplant rejections, cell proliferative disorders, cardiovascular disorders, disorders of the central nervous system and hepatitis C. Compounds represented by the structural formula (I) are also useful for the prevention or treatment of a TNF-α-related disorder in a mammal such as, but not limited to:
septic or endotoxic shock,
TNF-α-mediated diseases,
pathologies and conditions associated with and/or induced by abnormal levels of TNF-α occurring in a systemic, localized or particular tissue type or location in the body of the mammal,
toxic effects of TNF-α and/or anti-cancer chemotherapeutic agents,
injuries after irradiation of a tissue of the mammal by radio-elements, and
cachexia.

Compounds represented by the structural formula (I) are also useful for the prevention or treatment of a disorder mediated by phosphodiesterase-4 activity in a mammal such as, but not limited to, erectile dysfunction.

In a further embodiment, the present invention relates to combined preparations containing at least one compound represented by the structural formula (I) and one or more drugs such as, but not limited to, immunosuppressant and/or immunomodulator drugs, antineoplastic drugs, anti-histamines, inhibitors of agents causative of allergic conditions, phosphodiesterase-4 inhibitors, and antiviral agents. In a further embodiment, the present invention relates to the prevention or treatment of the above-cited pathologic conditions by administering to the patient in need thereof an effective amount of a compound represented by the structural formula (I), optionally in the form of a pharmaceutical composition or a combined preparation with another suitable drug.

In another embodiment, the present invention relates to various processes and methods for making the novel pyrido(3,2-d)pyrimidine derivatives defined in the structural formula (I) as well as their pharmaceutically acceptable salts, N-oxides, solvates and stereoisomers, e.g. via one or more groups of tri-substituted pyrido(3,2-d)pyrimidine intermediates such as specified herein before.

In yet another embodiment, the present invention relates to the use of monosubstituted, disubstituted and trisubstituted pyrido(3,2-d)pyrimidines, whatever their substitution pattern (i.e. with a substitution pattern broader than that of structural formula (I) hereinabove, including substitution patterns of pyrido(3,2-d)pyrimidines disclosed in the section “Background of the Invention”), as phosphodiesterase-4 inhibitors. In a specific embodiment, such use includes a method of treatment of a disease mediated by phosphodiesterase-4 activity in a patient, comprising the administration of an effective amount, preferably a phosphodiesterase-4 inhibiting amount, of a pyrido(3,2-d)pyrimidine derivative. Such a disease includes, but is not limited to, erectile dysfunction, e.g. vasculogenic impotence, in a male individual.

In another embodiment the present invention relates to pyrido(3,2-d)pyrimidine

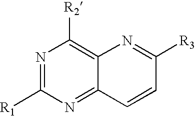

derivatives represented by the structural formula (II) (II) or the structural formula (III)

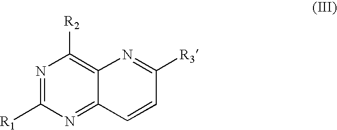

or the structural formula (IV)

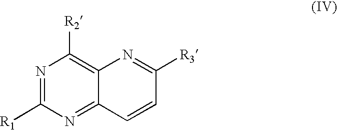

wherein:
R₁ is selected from the group consisting of hydrogen, halogen, cyano, carboxylic acid, acyl, thioacyl, alkoxycarbonyl, acyloxy, carbonate, carbamate, C₁₋₇ alkyl, aryl, amino, acetamido, N-protected amino, (mono- or di) C₁₋₇ alkylamino, (mono- or di) arylamino, (mono- or di) C₃₋₁₀ cycloalkylamino, (mono- or di) hydroxy C₁₋₇ alkylamino, (mono- or di) C₁₋₄ alkyl-arylamino, mercapto C₁₋₇ alkyl, C₁₋₇ alkyloxy, and groups of the formula R₆—NR₇R₁₂, wherein R₆ is a bond or C₁₋₃ alkylene, wherein R₇ and R₁₂ are independently selected from the group consisting of hydrogen, C₁₋₇ alkyl, C₂₋₇ alkenyl, C₂₋₇ alkynyl, aryl, arylalkyl, C₃₋₁₀ cycloalkyl and heteroaryl, or wherein R₇ and R₁₂ together form a heterocycle;
R₂ is selected from the group consisting of (mono- or di-) C₁₋₁₂ alkylamino; monoarylamino; diarylamino; (mono- or di-) C₃₋₁₀ cycloalkylamino; (mono- or di-) hydroxyC₁₋₇ alkylamino; (mono- or di-) C₁₋₄ alkylarylamino; (mono- or di-) arylC₁₋₄ alkylamino; morpholinyl; mercapto C₁₋₇ alkyl; C₁₋₇ alkoxy, homopiperazinyl and piperazinyl, wherein said homopiperazinyl or piperazinyl is optionally N-substituted with a substituent R₅
R₅ is selected from the group consisting of formyl, acyl, thioacyl, amide, thioamide, sulfonyl, sulfinyl, carboxylate, thiocarboxylate, amino-substituted acyl, alkoxyalkyl, C₃₋₁₀ cycloalkyl-alkyl, C₃₋₁₀ cycloalkyl, dialkylaminoalkyl, heterocyclic-substituted alkyl, acyl-substituted alkyl, thioacyl-substituted alkyl, amido-substituted alkyl, thioamido-substituted alkyl, carboxylato-substituted alkyl, thiocarboxylato-substituted alkyl, (amino-substituted acyl)alkyl, heterocyclic, carboxylic acid ester, w-cyanoalkyl, w-carboxylic ester-alkyl, halo C₁₋₇ alkyl, C₂₋₇ alkenyl, C₂₋₇ alkynyl, arylalkenyl, aryloxyalkyl, arylalkyl and aryl, wherein the aryl moiety of each of said arylalkenyl, aryloxyalkyl, arylalkyl and aryl radicals is optionally substituted with one or more substituents independently selected from the group consisting of halogen, C₁₋₇ alkyl, C₂₋₇ alkenyl, C₂₋₇ alkynyl, halo C₁₋₇ alkyl, nitro, hydroxyl, sulfhydryl, amino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thio-heterocyclic, arylalkylthio, heterocyclic-substituted alkylthio, formyl, carbamoyl, thiocarbamoyl, ureido, thioureido, sulfonamido, hydroxylamino, alkoxy-amino, mercaptoamino, thioalkylamino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazino, alkylhydrazino and phenylhydrazino;
R₃ is independently selected from the group consisting of hydrogen, heteroaryl and aryl groups, wherein said heteroaryl or aryl groups are optionally substituted with one or more substituents selected from the group consisting of halogen, C₁₋₇ alkyl, C₂₋₇ alkenyl, C₂₋₇ alkynyl, halo C₁₋₇ alkyl, nitro, hydroxyl, sulfhydryl, amino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thio-heterocyclic, arylalkylthio, heterocyclic-substituted alkylthio, formyl, carbamoyl, thiocarbamoyl, ureido, thioureido, sulfonamido, hydroxylamino, alkoxy-amino, mercaptoamino, thioalkylamino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazino, alkylhydrazino and phenylhydrazino;
R₂′ is selected from the group consisting of:
   piperazinyl or homopiperazinyl wherein one or more carbon atoms of said piperazinyl or homopiperazinyl are independently substituted with C₁₋₄ alkyl, or two carbon atoms of said piperazinyl or homopiperazinyl together with their alkyl substituent form a C₁₋₄ alkylene group, and wherein said piperazinyl or homopiperazinyl is optionally N-substituted with R₅;
   piperidin-4-yl-amino, piperidin-3-yl-amino, piperidin-4-yl-oxy, pyrrolidin-1-yl, 3-amino-pyrrolidin-1-yl, pyrrolidin-3-yl-amino or 2,6-diazabicyclo[3.2.0]heptan-2-yl, wherein said piperidin-4-yl, piperidin-3-yl, 3-amino-pyrrolidin-1-yl, pyrrolin-3-yl or 2,6-diazabicyclo[3.2.0]heptan-2-yl is optionally N-substituted with R₅ or C₁₋₄alkylarylcarbamoyl; or wherein any carbon atom of said piperidin-1-yl may be further substituted with one or more substituent selected from the group consisting of hydrocy, aryl, C₁₋₄alkylcarbamoyl, C₁₋₄alkoxycarbonyl and C₁₋₄alkylarylcarbamoyl;
   piperazinyl or homopiperazinyl being N-substituted with a substituent selected from the group consisting of C₁₋₄alkyl; arylcarbamoyl-substituted alkanoyl; arylalkanoyl wherein alkanoyl is substituted with one or more substituents selected from the group consisting of amino, hydroxy and halogen; mono-C₁₋₄alkylaryl-carbamoyl; di-C₁₋₄alkylaryl-carbamoyl; tri-C₁₋₄alkylaryl-carbamoyl; mono-C₁₋₄alkylaryl-C₁₋₄alkylcarbamoyl; di-C₁₋₄alkylaryl-C₁₋₄alkylcarbamoyl; tri-C₁₋₄alkylaryl-C₁₋₄alkylcarbamoyl; alkoxycarbonyl; alkanoyl substituted with one or more substituents independently selected from the group consisting of amino, alkoxycarbonyl, alkylcarbamate, arylamido and arylcarbamoyl; arylalkanoyl substituted by alkylcarbamate; cycloalkylcarbamoyl; alkoxyalkanoyl; dialkyl-carbamoyl; heterocyclic carbamoyl C₁₋₄alkyl; arylC₁₋₄alkylcarbamoyl; heterocyclic carbonyl C₁₋₄alkyl and aryl C₁₋₄alkylcarbamoyl C₁₋₄alkyl;
   triazolyl; heterocyclic amino; heterocyclic C₁₋₄alkylamino; alkoxy C₁₋₄alkylamino; amino cycloalkylamino; amino C₂₋₁₄alkylamino; amino C₁₋₆alkylamino wherein the N-atom is further substituted with C₁₋₄alkylarylcarbamoyl or aryloxy C₁₋₄alkanoyl; aryl C₁₋₄alkoxy; 3-amino-pyrrolidin-1-yl; N—C₁₋₄alkyl-N-arylcarbamoyl; and; C₁₋₄alkyl or alkanoyl substituted heterocyclic carbonyl C₁₋₄alkylamino; and,
R₃′ is an aryl group substituted with one or more substituents selected from the group consisting of heterocyclic; C₃₋₁₀ cycloalkylcarbamoyl; C₁₋₄ alkylcarbamoyl; C₁₋₄ alkylsulfonyl; C₁₋₄ alkylsulfonamido; C₁₋₄ alkyl-carboxylate; C₁₋₄ alkyl and C₁₋₄ alkoxy substituted with one or more substituents selected from the group consisting of amino, halogen, cyano and C₁₋₄alkoxy;
   or a pharmaceutical acceptable addition salt or a stereochemical isomeric form thereof or a N-oxide thereof or a solvate thereof.

In yet another embodiment the present invention relates to pharmaceutical compositions comprising a pyrido(3,2-d)pyrimidine derivative represented by one of the structural formulae (II), (III) and (IV) as an active ingredient especially for the treatment of immune disorders or the prevention of a transplant rejection.

### BRIEF DESCRIPTION OF THE DRAWINGS

FIG. 1 schematically shows a first method for making 2,4,6-tri-substituted pyrido(3,2-d)pyrimidine derivatives represented by the structural formula (I) wherein the substituent in position 2 is amino, as well as intermediates therefor wherein the substituent in position 2 is a N-protected amino such as acetamido and/or wherein the substituent in position 4 is hydroxy, chloro or triazolyl.

FIG. 2 schematically shows a second method for making 2,4,6-tri-substituted pyrido(3,2-d)pyrimidine derivatives represented by the structural formula (I) wherein the substituent in position 2 is amino, as well as intermediates therefor wherein the substituent in position 2 is a N-protected amino such as acetamido and/or wherein the substituent in position 4 is hydroxy, chloro or triazolyl.

FIG. 3 schematically shows a method for making 2,4,6-tri-substituted pyrido(3,2-d)pyrimidine intermediates represented by the structural formula (I), as well as intermediates wherein the substituent in position 4 is hydroxy, chloro or triazolyl.

FIG. 4 schematically shows another method for making 2,4,6-tri-substituted pyrido(3,2-d)pyrimidine intermediates represented by the structural formula (I), as well as intermediates wherein the substituent in positions 2 and 4 are hydroxy or chloro.

FIG. 5 schematically shows a first method for making 2,4,7-tri-substituted pyrido(3,2-d)pyrimidine derivatives represented by the structural formula (I) wherein the substituent in position 2 is amino, as well as intermediates therefor wherein the substituent in position 2 is a N-protected amino such as acetamido and/or wherein the substituent in position 4 is hydroxy, chloro or triazolyl.

FIG. 6 schematically shows a second method for making 2,4,7-tri-substituted pyrido(3,2-d)pyrimidine derivatives represented by the structural formula (I) wherein the substituent in position 2 is amino, as well as intermediates therefor wherein the substituent in position 2 is a N-protected amino such as acetamido and/or wherein the substituent in position 4 is hydroxy, chloro or triazolyl.

FIG. 7 schematically shows a method for making 2,4,7-tri-substituted pyrido(3,2-d)pyrimidine intermediates represented by the structural formula (I), as well as intermediates wherein the substituent in position 4 is hydroxy, chloro or triazolyl.

FIG. 8 schematically shows another method for making 2,4,7-tri-substituted pyrido(3,2-d)pyrimidine intermediates represented by the structural formula (I), as well as intermediates wherein the substituent in positions 2 and 4 are hydroxy or chloro.

### DEFINITIONS

Unless otherwise stated herein, the term “tri-substituted” means that three of the carbon atoms being in positions 2, 4 and 6 or, alternatively, in positions 2, 4 and 7 of the pyrido(3,2-d)pyrimidine moiety (according to standard atom numbering for the pyrido(3,2-d)pyrimidine moiety) are substituted with an atom or group of atoms other than hydrogen. The term “tetra-substituted” means that all four carbon atoms being in positions 2, 4, 6 and 7 of the pyrido(3,2-d)pyrimidine moiety are substituted with an atom or group of atoms other than hydrogen.

As used herein with respect to a substituting radical, and unless otherwise stated, the term “C₁₋₇ alkyl” means straight and branched chain saturated acyclic hydrocarbon monovalent radicals having from 1 to 7 carbon atoms such as, for example, methyl, ethyl, propyl, n-butyl, 1-methylethyl (isopropyl), 2-methylpropyl (isobutyl), 1,1-dimethylethyl (ter-butyl), 2-methylbutyl, n-pentyl, dimethylpropyl, n-hexyl, 2-methylpentyl, 3-methylpentyl, n-heptyl and the like. By analogy, the term C₁₋₁₂ alkyl” refers to such radicals having from 1 to 12 carbon atoms, i.e. up to and including dodecyl.

As used herein with respect to a substituting radical, and unless otherwise stated, the term “acyl” broadly refers to a substituent derived from an acid such as an organic monocarboxylic acid, a carbonic acid, a carbamic acid (resulting into a carbamoyl substituent) or the thioacid or imidic acid (resulting into a carbamidoyl substituent) corresponding to said acids, and the term “sulfonyl” refers to a substituent derived from an organic sulfonic acid, wherein said acids comprise an aliphatic, aromatic or heterocyclic group in the molecule. A more specific kind of “acyl” group within the scope of the above definition refers to a carbonyl (oxo) group adjacent to a C₁₋₇ alkyl, a C₃₋₁₀ cycloalkyl, an aryl, an arylalkyl or a heterocyclic group, all of them being such as herein defined. Suitable examples of acyl groups are to be found below.

Acyl and sulfonyl groups originating from aliphatic or cycloaliphatic monocarboxylic acids are designated herein as aliphatic or cycloaliphatic acyl and sulfonyl groups and include, but are not limited to, the following:
alkanoyl (for example formyl, acetyl, propionyl, butyryl, isobutyryl, valeryl, isovaleryl, pivaloyl and the like);
cycloalkanoyl (for example cyclobutanecarbonyl, cyclopentanecarbonyl, cyclohexanecarbonyl, 1-adamantanecarbonyl and the like);
cycloalkyl-alkanoyl (for example cyclohexylacetyl, cyclopentylacetyl and the like);
alkenoyl (for example acryloyl, methacryloyl, crotonoyl and the like);
alkylthioalkanoyl (for example methylthioacetyl, ethylthioacetyl and the like);
alkanesulfonyl (for example mesyl, ethanesulfonyl, propanesulfonyl and the like);
alkoxycarbonyl (for example methoxycarbonyl, ethoxycarbonyl, propoxycarbonyl, isopropoxycarbonyl, butoxycarbonyl, isobutoxycarbonyl and the like);
alkylcarbamoyl (for example methylcarbamoyl and the like);
(N-alkyl)-thiocarbamoyl (for example (N-methyl)-thiocarbamoyl and the like);
alkylcarbamidoyl (for example methylcarbamidoyl and the like); and
alkoxalyl (for example methoxalyl, ethoxalyl, propoxalyl and the like);

Acyl and sulfonyl groups may also originate from aromatic monocarboxylic acids and include, but are not limited to, the following:
aroyl (for example benzoyl, toluoyl, xyloyl, 1-naphthoyl, 2-naphthoyl and the like);
aralkanoyl (for example phenylacetyl and the like);
aralkenoyl (for example cinnamoyl and the like);
aryloxyalkanoyl (for example phenoxyacetyl and the like);
arylthioalkanoyl (for example phenylthioacetyl and the like);
arylaminoalkanoyl (for example N-phenylglycyl, and the like);
arylsulfonyl (for example benzenesulfonyl, toluenesulfonyl, naphthalene sulfonyl and the like);
aryloxycarbonyl (for example phenoxycarbonyl, naphthyloxycarbonyl and the like);
aralkoxycarbonyl (for example benzyloxycarbonyl and the like);
arylcarbamoyl (for example phenylcarbamoyl, naphthylcarbamoyl and the like);
arylglyoxyloyl (for example phenylglyoxyloyl and the like).
arylthiocarbamoyl (for example phenylthiocarbamoyl and the like); and
arylcarbamidoyl (for example phenylcarbamidoyl and the like).

Acyl groups may also originate from an heterocyclic monocarboxylic acids and include, but are not limited to, the following:
heterocyclic-carbonyl, in which said heterocyclic group is as defined herein, preferably an aromatic or non-aromatic 5- to 7-membered heterocyclic ring with one or more heteroatoms selected from the group consisting of nitrogen, oxygen and sulfur in said ring (for example thiophenoyl, furoyl, pyrrolecarbonyl, nicotinoyl and the like); and
heterocyclic-alkanoyl in which said heterocyclic group is as defined herein, preferably an aromatic or non-aromatic 5- to 7-membered heterocyclic ring with one or more heteroatoms selected from the group consisting of nitrogen, oxygen and sulfur in said ring (for example thiopheneneacetyl, furylacetyl, imidazolylpropionyl, tetrazolylacetyl, 2-(2-amino-4-thiazolyl)-2-methoxyiminoacetyl and the like).

As used herein with respect to a substituting radical, and unless otherwise stated, the term “thioacyl” refers to an acyl group as defined herein-above but wherein a sulfur atom replaces the oxygen atom of the carbonyl (oxo) moiety.

As used herein with respect to a substituting radical, and unless otherwise stated, the term “C₁₋₇ alkylene” means the divalent hydrocarbon radical corresponding to the above defined C₁₋₇ alkyl, such as methylene, bis(methylene), tris(methylene), tetramethylene, hexamethylene and the like.

As used herein with respect to a substituting radical, and unless otherwise stated, the term “C₃₋₁₀ cycloalkyl” means a mono- or polycyclic saturated hydrocarbon monovalent radical having from 3 to 10 carbon atoms, such as for instance cyclopropyl, cyclobutyl, cyclopentyl, cyclohexyl, cycloheptyl, cyclooctyl and the like, or a C₇₋₁₀ polycyclic saturated hydrocarbon monovalent radical having from 7 to 10 carbon atoms such as, for instance, norbornyl, fenchyl, trimethyltricycloheptyl or adamantyl.

As used herein with respect to a substituting radical, and unless otherwise stated, the term “C₃₋₁₀ cycloalkyl-alkyl” refers to an aliphatic saturated hydrocarbon monovalent radical (preferably a C₁₋₇ alkyl such as defined above) to which a C₃₋₁₀ cycloalkyl (such as defined above) is already linked such as, but not limited to, cyclohexylmethyl, cyclopentylmethyl and the like.

As used herein with respect to a substituting radical, and unless otherwise stated, the term “C₃₋₁₀ cycloalkylene” means the divalent hydrocarbon radical corresponding to the above defined C₃₋₁₀ cycloalkyl.

As used herein with respect to a substituting radical, and unless otherwise stated, the term “aryl” designate any mono- or polycyclic aromatic monovalent hydrocarbon radical having from 6 up to 30 carbon atoms such as but not limited to phenyl, naphthyl, anthracenyl, phenantracyl, fluoranthenyl, chrysenyl, pyrenyl, biphenylyl, terphenyl, picenyl, indenyl, biphenyl, indacenyl, benzocyclobutenyl, benzocyclooctenyl and the like, including fused benzo-C₄₋₈ cycloalkyl radicals (the latter being as defined above) such as, for instance, indanyl, tetrahydronaphtyl, fluorenyl and the like, all of the said radicals being optionally substituted with one or more substituents independently selected from the group consisting of halogen, amino, trifluoromethyl, hydroxyl, sulfhydryl and nitro, such as for instance 4-fluorophenyl, 4-chlorophenyl, 3,4-dichlorophenyl, 4-cyanophenyl, 2,6-dichlorophenyl, 2-fluorophenyl, 3-chlorophenyl, 3,5-dichlorophenyl and the like.

As used herein, e.g. with respect to a substituting radical such as the combination of substituents in certain positions of the pyrido(3,2-d)pyrimidine ring together with the carbon atoms in the same positions of said ring, and unless otherwise stated, the term “homocyclic” means a mono- or polycyclic, saturated or mono-unsaturated or polyunsaturated hydrocarbon radical having from 4 up to 15 carbon atoms but including no heteroatom in the said ring; for instance said combination of substituents may form a C₂₋₆ alkylene radical, such as tetramethylene, which cyclizes with the carbon atoms in certain positions of the pyrido(3,2-d)pyrimidine ring.

As used herein with respect to a substituting radical (including the combination of substituents in certain positions of the pyrido(3,2-d)pyrimidine ring together with the carbon atoms in the same positions of said ring), and unless otherwise stated, the term “heterocyclic” means a mono- or polycyclic, saturated or mono-unsaturated or polyunsaturated monovalent hydrocarbon radical having from 2 up to 15 carbon atoms and including one or more heteroatoms in one or more heterocyclic rings, each of said rings having from 3 to 10 atoms (and optionally further including one or more heteroatoms attached to one or more carbon atoms of said ring, for instance in the form of a carbonyl or thiocarbonyl or selenocarbonyl group, and/or to one or more heteroatoms of said ring, for instance in the form of a sulfone, sulfoxide, N-oxide, phosphate, phosphonate or selenium oxide group), each of said heteroatoms being independently selected from the group consisting of nitrogen, oxygen, sulfur, selenium and phosphorus, also including radicals wherein a heterocyclic ring is fused to one or more aromatic hydrocarbon rings for instance in the form of benzo-fused, dibenzo-fused and naphto-fused heterocyclic radicals; within this definition are included heterocyclic radicals such as, but not limited to, diazepinyl, oxadiazinyl, thiadiazinyl, dithiazinyl, triazolonyl, diazepinonyl, triazepinyl, triazepinonyl, tetrazepinonyl, benzoquinolinyl, benzothiazinyl, benzothiazinonyl, benzoxa-thiinyl, benzodioxinyl, benzodithiinyl, benzoxazepinyl, benzothiazepinyl, benzodiazepinyl, benzodioxepinyl, benzodithiepinyl, benzoxazocinyl, benzo-thiazocinyl, benzodiazocinyl, benzoxathiocinyl, benzodioxocinyl, benzotrioxepinyl, benzoxathiazepinyl, benzoxadiazepinyl, benzothia-diazepinyl, benzotriazepinyl, benzoxathiepinyl, benzotriazinonyl, benzoxazolinonyl, azetidinonyl, azaspiroundecyl, dithiaspirodecyl, selenazinyl, selenazolyl, selenophenyl, hypoxanthinyl, azahypo-xanthinyl, bipyrazinyl, bipyridinyl, oxazolidinyl, diselenopyrimidinyl, benzodioxocinyl, benzopyrenyl, benzopyranonyl, benzophenazinyl, benzoquinolizinyl, dibenzo-carbazolyl, dibenzoacridinyl, dibenzophenazinyl, dibenzothiepinyl, dibenzoxepinyl, dibenzopyranonyl, dibenzoquinoxalinyl, dibenzothiazepinyl, dibenzisoquinolinyl, tetraazaadamantyl, thiatetraazaadamantyl, oxauracil, oxazinyl, dibenzothiophenyl, dibenzofuranyl, oxazolinyl, oxazolonyl, azaindolyl, azolonyl, thiazolinyl, thiazolonyl, thiazolidinyl, thiazanyl, pyrimidonyl, thiopyrimidonyl, thiamorpholinyl, azlactonyl, naphtindazolyl, naphtindolyl, naphtothiazolyl, naphtothioxolyl, naphtoxindolyl, naphto-triazolyl, naphtopyranyl, oxabicycloheptyl, azabenzimidazolyl, azacycloheptyl, azacyclooctyl, azacyclononyl, azabicyclononyl, tetrahydrofuryl, tetrahydropyranyl, tetrahydro-pyronyl, tetrahydroquinoleinyl, tetrahydrothienyl and dioxide thereof, dihydrothienyl dioxide, dioxindolyl, dioxinyl, dioxenyl, dioxazinyl, thioxanyl, thioxolyl, thiourazolyl, thiotriazolyl, thiopyranyl, thiopyronyl, coumarinyl, quinoleinyl, oxyquinoleinyl, quinuclidinyl, xanthinyl, dihydropyranyl, benzodihydrofuryl, benzothiopyronyl, benzothiopyranyl, benzoxazinyl, benzoxazolyl, benzodioxolyl, benzodioxanyl, benzothiadiazolyl, benzotriazinyl, benzothiazolyl, benzoxazolyl, phenothioxinyl, phenothiazolyl, phenothienyl (benzothiofuranyl), phenopyronyl, phenoxazolyl, pyridinyl, dihydropyridinyl, tetrahydropyridinyl, piperidinyl, morpholinyl, thiomorpholinyl, pyrazinyl, pyrimidinyl, pyridazinyl, triazinyl, tetrazinyl, triazolyl, benzotriazolyl, tetrazolyl, imidazolyl, pyrazolyl, thiazolyl, thiadiazolyl, isothiazolyl, oxazolyl, oxadiazolyl, pyrrolyl, furyl, dihydrofuryl, furoyl, hydantoinyl, dioxolanyl, dioxolyl, dithianyl, dithienyl, dithiinyl, thienyl, indolyl, indazolyl, benzofuryl, quinolyl, quinazolinyl, quinoxalinyl, carbazolyl, phenoxazinyl, phenothiazinyl, xanthenyl, purinyl, benzothienyl, naphtothienyl, thianthrenyl, pyranyl, pyronyl, benzopyronyl, isobenzofuranyl, chromenyl, phenoxathiinyl, indolizinyl, quinolizinyl, isoquinolyl, phthalazinyl, naphthiridinyl, cinnolinyl, pteridinyl, carbolinyl, acridinyl, perimidinyl, phenanthrolinyl, phenazinyl, phenothiazinyl, imidazolinyl, imidazolidinyl, benzimidazolyi, pyrazolinyl, pyrazolidinyl, pyrrolinyl, pyrrolidinyl, piperazinyl, uridinyl, thymidinyl, cytidinyl, azirinyl, aziridinyl, diazirinyl, diaziridinyl, oxiranyl, oxaziridinyl, dioxiranyl, thiiranyl, azetyl, dihydroazetyl, azetidinyl, oxetyl, oxetanyl, oxetanonyl, homopiperazinyl, homopiperidinyl, thietyl, thietanyl, diazabicyclooctyl, diazetyl, diaziridinonyl, diaziridinethionyl, chromanyl, chromanonyl, thiochromanyl, thiochromanonyl, thiochromenyl, benzofuranyl, benzisothiazolyl, benzocarbazolyl, benzochromonyl, benzisoalloxazinyl, benzocoumarinyl, thiocoumarinyl, pheno-metoxazinyl, phenoparoxazinyl, phentriazinyl, thiodiazinyl, thiodiazolyl, indoxyl, thioindoxyl, benzodiazinyl (e.g. phtalazinyl), phtalidyl, phtalimidinyl, phtalazonyl, alloxazinyl, dibenzopyronyl (i.e. xanthonyl), xanthionyl, isatyl, isopyrazolyl, isopyrazolonyl, urazolyl, urazinyl, uretinyl, uretidinyl, succinyl, succinimido, benzylsultimyl, benzylsultamyl and the like, including all possible isomeric forms thereof, wherein each carbon atom of said heterocyclic ring may furthermore be independently substituted with a substituent selected from the group consisting of halogen, nitro, C₁₋₇ alkyl (optionally containing one or more functions or radicals selected from the group consisting of carbonyl (oxo), alcohol (hydroxyl), ether (alkoxy), acetal, amino, imino, oximino, alkyloximino, amino-acid, cyano, carboxylic acid ester or amide, nitro, thio C₁₋₇ alkyl, thio C₃₋₁₀ cycloalkyl, C₁₋₇ alkylamino, cycloalkylamino, alkenylamino, cycloalkenylamino, alkynylamino, arylamino, arylalkyl-amino, hydroxylalkylamino, mercaptoalkylamino, heterocyclic-substituted alkylamino, heterocyclic amino, heterocyclic-substituted arylamino, hydrazino, alkylhydrazino, phenylhydrazino, sulfonyl, sulfonamido and halogen), C₃₋₇ alkenyl, C₂₋₇ alkynyl, halo C₁₋₇ alkyl, C₃₋₁₀ cycloalkyl, aryl, arylalkyl, alkylaryl, alkylacyl, arylacyl, hydroxyl, amino, C₁₋₇ alkylamino, cycloalkylamino, alkenylamino, cycloalkenylamino, alkynylamino, arylamino, arylalkylamino, hydroxyalkylamino, mercaptoalkylamino, heterocyclic-substituted alkylamino, heterocyclic amino, heterocyclic-substituted arylamino, hydrazino, alkylhydrazino, phenylhydrazino, sulfhydryl, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thioheterocyclic, arylalkylthio, heterocyclic-substituted alkylthio, formyl, hydroxylamino, cyano, carboxylic acid or esters or thioesters or amides thereof, thiocarboxylic acid or esters or thioesters or amides thereof; depending upon the number of unsaturations in the 3 to 10 atoms ring, heterocyclic radicals may be sub-divided into heteroaromatic (or “heteroaryl”) radicals and non-aromatic heterocyclic radicals; when a heteroatom of said non-aromatic heterocyclic radical is nitrogen, the latter may be substituted with a substituent selected from the group consisting of C₁₋₇ alkyl, C₃₋₁₀ cycloalkyl, aryl, arylalkyl and alkylaryl.

As used herein with respect to a substituting radical, and unless otherwise stated, the terms “C₁₋₇ alkoxy”, “C₃₋₁₀ cycloalkoxy”, “aryloxy”, “arylalkyloxy”, “oxyheterocyclic”, “thio C₁₋₇ alkyl”, “thio C₃₋₁₀ cycloalkyl”, “arylthio”, “arylalkylthio” and “thioheterocyclic” refer to substituents wherein a carbon atom of a C₁₋₇ alkyl, respectively a C₃₋₁₀ cycloalkyl, aryl, arylalkyl or heterocyclic radical (each of them such as defined herein), is attached to an oxygen atom or a divalent sulfur atom through a single bond such as, but not limited to, methoxy, ethoxy, propoxy, butoxy, pentoxy, isopropoxy, sec-butoxy, tert-butoxy, isopentoxy, cyclopropyloxy, cyclobutyloxy, cyclopentyloxy, thiomethyl, thioethyl, thiopropyl, thiobutyl, thiopentyl, thiocyclopropyl, thiocyclobutyl, thiocyclopentyl, thiophenyl, phenyloxy, benzyloxy, mercaptobenzyl, cresoxy, and the like.

As used herein with respect to a substituting atom, and unless otherwise stated, the term halogen means any atom selected from the group consisting of fluorine, chlorine, bromine and iodine.

As used herein with respect to a substituting radical, and unless otherwise stated, the term “halo C₁₋₇ alkyl” means a C₁₋₇ alkyl radical (such as above defined) in which one or more hydrogen atoms are independently replaced by one or more halogens (preferably fluorine, chlorine or bromine), such as but not limited to difluoromethyl, trifluoromethyl, trifluoroethyl, octafluoropentyl, dodecafluoroheptyl, dichloromethyl and the like.

As used herein with respect to a substituting radical, and unless otherwise stated, the terms “C₂₋₇ alkenyl” designate a straight and branched acyclic hydrocarbon monovalent radical having one or more ethylenic unsaturations and having from 2 to 7 carbon atoms such as, for example, vinyl, 1-propenyl, 2-propenyl (allyl), 1-butenyl, 2-butenyl, 2-pentenyl, 3-pentenyl, 3-methyl-2-butenyl, 3-hexenyl, 2-hexenyl, 2-heptenyl, 1,3-butadienyl, pentadienyl, hexadienyl, heptadienyl, heptatrienyl and the like, including all possible isomers thereof.

As used herein with respect to a substituting radical, and unless otherwise stated, the term “C₃₋₁₀ cycloalkenyl” means a monocyclic mono- or polyunsaturated hydrocarbon monovalent radical having from 3 to 8 carbon atoms, such as for instance cyclopropenyl, cyclobutenyl, cyclopentenyl, cyclopentadienyl, cyclohexenyl, cyclohexadienyl, cycloheptenyl, cyclohepta-dienyl, cycloheptatrienyl, cyclooctenyl, cyclooctadienyl and the like, or a C₇₋₁₀ polycyclic mono- or polyunsaturated hydrocarbon mono-valent radical having from 7 to 10 carbon atoms such as dicyclopentadienyl, fenchenyl (including all isomers thereof, such as α-pinolenyl), bicyclo[2.2.1]hept-2-enyl, bicyclo[2.2.1]hepta-2,5-dienyl, cyclo-fenchenyl and the like.

As used herein with respect to a substituting radical, and unless otherwise stated, the term “C₂₋₇ alkynyl” defines straight and branched chain hydrocarbon radicals containing one or more triple bonds and optionally at least one double bond and having from 2 to 7 carbon atoms such as, for example, acetylenyl, 1-propynyl, 2-propynyl, 1-butynyl, 2-butynyl, 2-pentynyl, 1-pentynyl, 3-methyl-2-butynyl, 3-hexynyl, 2-hexynyl, 1-penten-4-ynyl, 3-penten-1-ynyl, 1,3-hexadien-1-ynyl and the like.

As used herein with respect to a substituting radical, and unless otherwise stated, the terms “arylalkyl”, “arylalkenyl” and “heterocyclic-substituted alkyl” refer to an aliphatic saturated or ethylenically unsaturated hydrocarbon monovalent radical (preferably a C₁₋₇ alkyl or C₂₋₇ alkenyl radical such as defined above) onto which an aryl or heterocyclic radical (such as defined above) is already bonded via a carbon atom, and wherein the said aliphatic radical and/or the said aryl or heterocyclic radical may be optionally substituted with one or more substituents independently selected from the group consisting of halogen, amino, hydroxyl, sulfhydryl, C₁₋₇ alkyl, C₁₋₇ alkoxy, trifluoromethyl and nitro, such as but not limited to benzyl, 4-chlorobenzyl, 4-fluorobenzyl, 2-fluorobenzyl, 3,4-dichlorobenzyl, 2,6-dichlorobenzyl, 3-methylbenzyl, 4-methylbenzyl, 4-ter-butylbenzyl, phenylpropyl, 1-naphthylmethyl, phenylethyl, 1-amino-2-phenylethyl, 1-amino-2-[4-hydroxy-phenyl]ethyl, 1-amino-2-[indol-2-yl]ethyl, styryl, pyridylmethyl (including all isomers thereof), pyridylethyl, 2-(2-pyridyl)isopropyl, oxazolylbutyl, 2-thienylmethyl, pyrrolylethyl, morpholinylethyl, imidazol-1-yl-ethyl, benzodioxolylmethyl and 2-furylmethyl.

As used herein with respect to a substituting radical, and unless otherwise stated, the terms “alkylaryl” and “alkyl-substituted heterocyclic” refer to an aryl or, respectively, heterocyclic radical (such as defined above) onto which are bonded one or more aliphatic saturated or unsaturated hydrocarbon monovalent radicals, preferably one or more C₁₋₇ alkyl, C₂₋₇ alkenyl or C₃₋₁₀ cycloalkyl radicals as defined above such as, but not limited to, o-toluoyl, m-toluoyl, p-toluoyl, 2,3-xylyl, 2,4-xylyl, 3,4-xylyl, o-cumenyl, m-cumenyl, p-cumenyl, o-cymenyl, m-cymenyl, p-cymenyl, mesityl, ter-butylphenyl, lutidinyl (i.e. dimethylpyridyl), 2-methylaziridinyl, methyl-benzimidazolyl, methylbenzofuranyl, methylbenzothiazolyl, methylbenzotriazolyl, methylbenzoxazolyl and methylbenzselenazolyl.

As used herein with respect to a substituting radical, and unless otherwise stated, the term “alkoxyaryl” refers to an aryl radical (such as defined above) onto which is (are) bonded one or more C₁₋₇ alkoxy radicals as defined above, preferably one or more methoxy radicals, such as, but not limited to, 2-methoxyphenyl, 3-methoxyphenyl, 4-methoxyphenyl, 3,4-dimethoxyphenyl, 2,4,6-trimethoxyphenyl, methoxynaphtyl and the like.

As used herein with respect to a substituting radical, and unless otherwise stated, the terms “alkylamino”, “cycloalkylamino”, “alkenylamino”, “cyclo-alkenylamino”, “arylamino”, “arylalkylamino”, “heterocyclic-substituted alkyl-amino”, “heterocyclic-substituted arylamino”, “heterocyclic amino”, “hydroxy-alkylamino”, “mercaptoalkylamino” and “alkynylamino” mean that respectively one (thus monosubstituted amino) or even two (thus disubstituted amino) C₁₋₇ alkyl, C₃₋₁₀ cycloalkyl, C₂₋₇ alkenyl, C₃₋₁₀ cycloalkenyl, aryl, arylalkyl, heterocyclic-substituted alkyl, heterocyclic-substituted aryl, heterocyclic (provided in this case the nitrogen atom is attached to a carbon atom of the heterocyclic ring), mono- or polyhydroxy C₁₋₇ alkyl, mono- or polymercapto C₁₋₇ alkyl, or C₂₋₇ alkynyl radical(s) (each of them as defined herein, respectively, and including the presence of optional substituents independently selected from the group consisting of halogen, amino, hydroxyl, sulfhydryl, C₁₋₇ alkyl, C₁₋₇ alkoxy, trifluoromethyl and nitro) is/are attached to a nitrogen atom through a single bond such as, but not limited to, anilino, 2-bromoanilino, 4-bromoanilino, 2-chloroanilino, 3-chloroanilino, 4-chloroanilino, 3-chloro-4-methoxyanilino, 5-chloro-2-methoxyanilino, 2,3-dimethylanilino, 2,4-dimethylanilino, 2,5-dimethylanilino, 2,6-dimethylanilino, 3,4-dimethylanilino, 2-fluoroanilino, 3-fluoroanilino, 4-fluoroanilino, 3-fluoro-2-methoxyanilino, 3-fluoro-4-methoxyanilino, 2-fluoro-4-methylanilino, 2-fluoro-5-methylanilino, 3-fluoro-2-methylanilino, 3-fluoro-4-methylanilino, 4-fluoro-2-methylanilino, 5-fluoro-2-methylanilino, 2-iodoanilino, 3-iodoanilino, 4-iodoanilino, 2-methoxy-5-methylanilino, 4-methoxy-2-methylanilino, 5-methoxy-2-methylanilino, 2-ethoxyanilino, 3-ethoxy-anilino, 4-ethoxyanilino, benzylamino, 2-methoxybenzylamino, 3-methoxybenzylamino, 4-methoxybenzylamino, 2-fluorobenzylamino, 3-fluorobenzylamino, 4-fluoro-benzylamino, 2-chlorobenzylamino, 3-chlorobenzylamino, 4-chlorobenzylamino, 2-aminobenzylamino, diphenylmethylamino, α-naphthylamino, methylamino, dimethylamino, ethylamino, diethylamino, isopropylamino, propenylamino, n-butylamino, ter-butylamino, dibutylamino, 1,2-diaminopropyl, 1,3-diaminopropyl, 1,4-diaminobutyl, 1,5-diaminopentyl, 1,6-diaminohexyl, morpholinomethylamino, 4-morpholinoanilino, hydroxymethylamino, β-hydroxyethylamino and ethynylamino; this definition also includes mixed disubstituted amino radicals wherein the nitrogen atom is attached to two such radicals belonging to two different sub-sets of radicals, e.g. an alkyl radical and an alkenyl radical, or to two different radicals within the same sub-set of radicals, e.g. methylethylamino; among di-substituted amino radicals, symmetrically-substituted amino radicals are more easily accessible and thus usually preferred from a standpoint of ease of preparation.

As used herein with respect to a substituting radical, and unless otherwise stated, the terms “(thio)carboxylic acid ester”, “(thio)carboxylic acid thioester” and “(thio)carboxylic acid amide” refer to radicals wherein the carboxyl or thiocarboxyl group is bonded to the hydrocarbonyl residue of an alcohol, a thiol, a polyol, a phenol, a thiophenol, a primary or secondary amine, a polyamine, an amino-alcohol or ammonia, the said hydrocarbonyl residue being selected from the group consisting of alkyl, alkenyl, alkynyl, cycloalkyl, cycloalkenyl, aryl, arylalkyl, alkylaryl, alkylamino, cycloalkylamino, alkenylamino, cycloalkenylamino, arylamino, arylalkylamino, heterocyclic-substituted alkylamino, heterocyclic amino, heterocyclic-substituted arylamino, hydroxyalkylamino, mercapto-alkylamino or alkynylamino (such as above defined, respectively).

As used herein with respect to a substituting radical, and unless otherwise stated, the term “amino-acid” refers to a radical derived from a molecule having the chemical formula H₂N—CHR—COOH, wherein R is the side group of atoms characterising the amino-acid type; said molecule may be one of the 20 naturally-occurring amino-acids or any similar non naturally-occurring amino-acid.

As used herein and unless otherwise stated, the term “stereoisomer” refers to all possible different isomeric as well as conformational forms which the compounds of formula (I) may possess, in particular all possible stereochemically and conformationally isomeric forms, all diastereomers, enantiomers and/or conformers of the basic molecular structure. Some compounds of the present invention may exist in different tautomeric forms, all of the latter being included within the scope of the present invention.

As used herein and unless otherwise stated, the term “enantiomer” means each individual optically active form of a compound of the invention, having an optical purity or enantiomeric excess (as determined by methods standard in the art) of at least 80% (i.e. at least 90% of one enantiomer and at most 10% of the other enantiomer), preferably at least 90% and more preferably at least 98%.

As used herein and unless otherwise stated, the term “solvate” includes any combination which may be formed by a pyrido(3,2-d)pyrimidine derivative of this invention with a suitable inorganic solvent (e.g. hydrates) or organic solvent, such as but not limited to alcohols, ketones, esters, ethers, nitriles and the like.

### DETAILED DESCRIPTION OF THE INVENTION

In the first embodiment of the invention, the novel pyrido(3,2-d)pyrimidine derivatives are as defined in the general formula (I), wherein each of the substituents R₁, R₂, R₃ and/or R₄ may independently correspond to any of the definitions given above, in particular with any of the individual meanings (such as illustrated above) of generic terms used for substituting radicals such as, but not limited to, “C₁₋₇ alkyl”, “C₃₋₁₀ cycloalkyl”, “C₂₋₇ alkenyl”, “C₂₋₇ alkynyl”, “aryl”, “homocyclic heterocyclic”, “halogen”, “C₃₋₁₀ cycloalkenyl”, “alkylaryl”, “arylalkyl “alkylamino”, “cycloalkyl-amino”, “alkenylamino”, “alkynylamino”, “arylamino”, arylalkylamino”, “heterocyclic-substituted alkylamino”, “heterocyclic amino heterocyclic-substituted arylamino”, “hydroxyalkylamino”, “mercaptoalkylamino”, “alkynylamino”, “C₁₋₇ alkoxy”, “C₃₋₁₀ cycloalkoxy”, “thio C₁₋₇ alkyl”, “thio C₃₋₁₀ cycloalkyl”, “halo C₁₋₇ alkyl” and the like.

In the second embodiment of the invention, the novel pyrido(3,2-d)pyrimidine intermediates are as specified herein before, wherein each of the substituents R₁, R₂, R₃ and/or R₄ may independently correspond to any of the definitions given with respect to the general formula (I), in particular with any of the individual meanings (such as illustrated above) of generic terms used for substituting radicals such as, but not limited to, “C₁₋₇ alkyl”, “C₃₋₁₀ cycloalkyl”, “C₂₋₇ alkenyl”, “C₂₋₇ alkynyl”, “aryl”, “homocyclic”, “heterocyclic”, “halogen”, “C₃₋₁₀ cycloalkenyl”, “alkylaryl”, “aryl-alkyl”, “alkylamino”, “cycloalkylamino”, “alkenylamino”, “alkynylamino”, “aryl-amino”, “arylalkylamino”, “heterocyclic-substituted alkylamino”, “heterocyclic amino”, “heterocyclic-substituted arylamino”, “hydroxyalkylamino”, “mercapto-alkylamino”, “alkynylamino”, “C₁₋₇ alkoxy”, “C₃₋₁₀ cycloalkoxy”, “thio C₁₋₇ alkyl” “thio C₃₋₁₀ cycloalkyl”, “halo C₁₋₇ alkyl” and the like.

In another embodiment of the present invention, the novel pyrido(3,2-d)pyrimidine derivatives are as defined in one of the structural formulae (II), (III) and (IV) wherein each of the substituents R₁, R₂, R₂′, R₃, R₃′ and/or R₅ may independently correspond to any of the definitions given above, in particular with any of the above illustrated individual meanings of generic terms used for substituting radicals such as but not limited to “C₁₋₇ alkyl”, “C₃₋₁₀ cycloalkyl”, “C₂₋₇ alkenyl”, “C₂₋₇ alkynyl”, “acyl”, “thioacyl”, “aryl”, “heterocyclic”, “halogen”, “alkylaryl”, “arylalkyl”, “alkylamino”, “cycloalkylamino”, “arylamino”, “aryl C₁₋₄ alkylamino”, “C₁₋₄ alkylarylamino”, “hydroxy C₁₋₇ alkylamino”, “thioalkylamino”, “C₁₋₇ alkoxy”, “C₃₋₁₀ cycloalkoxy”, “aryloxy”, “thio C₁₋₇ alkyl”, “thio C₃₋₁₀ cycloalkyl”, “thioaryl”, “halo C₁₋₇ alkyl” and the like.

Within the class of compounds represented by the structural formula (I), a preferred group is one wherein R₂ is a piperazinyl group optionally N-substituted with a substituent R₅ such as defined herein above. Said piperazinyl group may be further substituted, at one or more carbon atoms, by a number n of substituents R₀ wherein n is an integer from 0 to 6 and wherein, when n is at least 2, each R₀ may be defined independently from the others. The presence of one or more such substituents R₀ at one or more carbon atoms is a suitable way for introducing chirality into the pyrido(2,3-d)pyrimidine derivatives represented by the structural formula (I) as well as into the corresponding intermediates. In practice, the choice of such substituents R₀ may be restricted by the commercial availability of the substituted piperazine. More preferably R₂ is a piperazin-1-yl group, n is 0, 1 or 2, and a representative example of the substituent R₀ is methyl or phenyl such as for instance in 2-methylpiperazin-1-yl, 2-phenylpiperazin-1-yl and 2,5-dimethyl-piperazin-1-yl. Within the preferred group of compounds, a more specific embodiment of the invention is one wherein one of the two nitrogen atoms of the piperazinyl group bears a substituent R₅ which has a carbonyl (oxo) or thiocarbonyl (thioxo) or sulfonyl function preferably immediately adjacent to the said nitrogen atom. In other words, this specific embodiment means that when R₅ is selected from, respectively, acyl, thioacyl, amide, thioamide, sulfonyl, sulfinyl, carboxylate and thiocarboxylate, then R₅ together with the nitrogen atom to which it is attached forms, respectively, an amide, thioamide, urea, thiourea, sulfonamido, sulfinamido, carbamato or thiocarbamato group.

Especially useful species of pyrido(3,2-d)pyrimidine derivatives represented by the structural formula (I) are those wherein the substituent R₂ is a piperazin-1-yl group, said group being substituted in the 4 position with a substituent R₅, wherein R₅ is selected from the group consisting of:
COR₈ wherein R₈ is selected from hydrogen; C₁₋₇ alkyl; C₃₋₁₀ cycloalkyl; aryl optionally substituted with one or more substituents selected from the group consisting of halogen, C₁₋₇ alkyl, cyano and C₁₋₇ alkoxy; heterocyclic optionally substituted with one or more halogen atoms; arylalkyl; aryloxyalkyl; arylalkoxyalkyl; alkoxyalkyl; arylalkoxy; aryloxy; arylalkenyl; heterocyclic-substituted alkyl; alkylamino and arylamino; representative but non limiting examples of R₈ are methyl, ethyl, pentyl, cyclohexyl, phenyl, 4-fluorophenyl, 4-chlorophenyl, 3,4-dichlorophenyl, 4-butylphenyl, 4-cyanophenyl, 2-methoxyphenyl, 3-methoxyphenyl, 4-pentoxyphenyl, naphtyl, 2-thienyl, 4-pyridinyl, 1-tetrahydropyrrolyl, 2-tetrahydropyrrolyl, 2-furanyl, 3-furanyl, 2,4-dichloro-5-fluoro-3-pyridinyl, diethylamino, diisopropylamino, diphenylamino, phenyl-ethyl, 4-chlorobenzyl, phenoxymethyl, benzyloxymethyl, methoxymethyl, 2-thienylmethyl, styryl, benzyloxy, phenoxy, 1-amino-2-phenylethyl, 1-amino-2-[4-hydroxyphenyl]ethyl and 1-amino-2-[indol-2-yl]ethyl;
CSR₉, wherein R₉ is selected from the group consisting of alkylamino and aryloxy, such as but not limited to dimethylamino and phenoxy;
SO₂R₁₀, wherein R₁₀ is selected from the group consisting of aryl and arylalkyl, such as but not limited to phenyl and benzyl; and
R₁₁, wherein R₁₁ is selected from the group consisting of C₁₋₇ alkyl, aryl, arylalkyl, arylalkenyl, alkoxyalkyl, heterocyclic-substituted alkyl, cycloalkylalkyl, heterocyclic, C₃₋₁₀ cycloalkyl, alkylaminoalkyl, aryloxyalkyl, alkoxyaryl, ω-cyanoalkyl, ω-carboxylatoalkyl and carboxamidoalkyl.

Especially useful species of pyrido(3,2-d)pyrimidine derivatives represented by the structural formula (I) are those wherein the substituent R₁ is a group represented by the structural formula R₆—NR₇R₁₂, wherein R₆ is a bond or C₁₋₃ alkylene, wherein R₇ and R₁₂ are independently selected from the group consisting of hydrogen, C₁₋₇ alkyl, C₂₋₇ alkenyl, C₂₋₇ alkynyl, aryl, arylalkyl, C₃₋₁₀ cycloalkyl and heteroaryl, or wherein N, R₇ and R₁₂ together form a heterocycle. Within this sub-class of derivatives, it is preferred when R₆ is a bond or methylene, and/or R₇ is methyl, ethyl, propyl or cyclopropylmethyl, and/or N, R₇ and R₁₂ together form morpholinyl, 2,6-dimethylmorpholinyl, pyrrolidinyl, azepanyl, 3,3,5-trimethylazepanyl, piperidinyl, 2-methylpiperidinyl or 2-ethylpiperidinyl. Methods for introducing such substituents in position 2 of the pyrido(3,2-d)pyrimidine ring are extensively described in WO 03/062209.

The present invention further provides various processes and methods for making the novel pyrido(3,2-d)pyrimidine derivatives represented by the structural formula (I). As a general rule, the preparation of these compounds is based on the principle that, starting from a suitable pyrido(3,2-d)pyrimidine precursor (usually a 2,3,6-trisubstituted pyridine), each of the substituents R₂, R₃, R₄ and R₁ may be introduced separately without adversely influencing the presence of one or more substituents already introduced at other positions on the pyrido(3,2-d)pyrimidine moiety or the capacity to introduce further substituents later on.

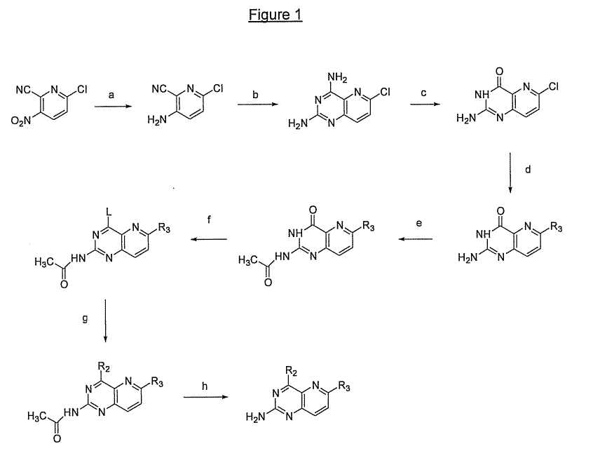

Methods of manufacture have been developed by the present inventors which may be used alternatively to, or may be combined with, the methods of synthesis already known in the art of pyrido(3,2-d)pyrimidine derivatives (depending upon the targeted final compound). For instance, the synthesis of mono- and di-N-oxides of the pyrido(3,2-d)pyrimidine derivatives of this invention can easily be achieved by treating the said derivatives with an oxidizing agent such as, but not limited to, hydrogen peroxide (e.g. in the presence of acetic acid) or a peracid such as chloroperbenzoic acid. The methods for making the pyrido(3,2-d)pyrimidine derivatives of the present invention will now be explained in more details by reference to the appended FIGS. 1 to 8 wherein, unless otherwise stated hereinafter, each of the substituting groups or atoms R₂, R₃, R₄ and R₁ is as defined in the structural formula (I) of the summary of the invention and, more specifically, may correspond to any of the individual meanings disclosed above.

In the description of the reaction steps involved in each figure, reference is made to the use of certain catalysts and/or certain types of solvents. It should be understood that each catalyst mentioned should be used in a catalytic amount well known to the skilled person with respect to the type of reaction involved. Solvents that may be used in the following reaction steps include various kinds of organic solvents such as protic solvents, polar aprotic solvents and non-polar solvents as well as aqueous solvents which are inert under the relevant reaction conditions. More specific examples include aromatic hydrocarbons, chlorinated hydrocarbons, ethers, aliphatic hydrocarbons, alcohols, esters, ketones, amides, water or mixtures thereof, as well as supercritical solvents such as carbon dioxide (while performing the reaction under supercritical conditions). The suitable reaction temperature and pressure conditions applicable to each kind of reaction step will not be detailed herein but do not depart from the relevant conditions already known to the skilled person with respect to the type of reaction involved and the type of solvent used (in particular its boiling point).

FIG. 1 schematically shows a first method for making 2,4,6-tri-substituted pyrido(3,2-d)pyrimidine derivatives represented by the structural formula (I) wherein the substituent in position 2 is amino, as well as intermediates therefor wherein the substituent in position 2 is a N-protected amino such as acetamido and/or wherein the substituent in position 4 is hydroxy, chloro or triazolyl. The nitro group of 6-chloro-2-cyano-3-nitropyridine is reduced in step (a) either catalytically (e.g. by using platinum or palladium under an atmosphere of hydrogen) or chemically (e.g. by using iron or tin under acidic conditions). A ring closure reaction leading to the formation of the pyrido[3,2-d]pyrimidine scaffold occurs in step (b) by treatment of 6-chloro-2-cyano-3-aminopyridine with a ring closure reagent such as, but not limited to, chloroformamidine or guanidine. Aqueous hydrolysis under aqueous acidic conditions then yields 2-amino-6-chloro-pyrido[3,2-d]pyrimidin-4(3H)one in step (c). In step (d), the chlorine atom at position 6 can be used as a leaving group for a variety of palladium-catalyzed reactions such as, but not limited to, a Suzuki reaction (by treatment of 2-amino-6-chloro-pyrido[3,2-d]pyrimidin-4(3H)one with an arylboronic or heteroarylboronic acid, or an ester thereof, leading to the formation of a biaryl derivative) and a Heck reaction (by treatment of 2-amino-6-chloro-pyrido[3,2-d]pyrimidin-4(3H)one with a wide variety of terminal alkenes or alkynes, thus yielding alkenyl or alkynyl compounds). In step (e), the amino group at position 2 is protected, for example by a pivaloyl (not shown in FIG. 1) or acetyl group, by reaction with acetic anhydride or pivaloyl anhydride in pyridine as a solvent, thus resulting into the introduction of a N-protected amino group at position 2 such as, but not limited to, acetamido or pivalamido. Activation of the tautomeric hydroxyl group at position 4 of the pyrido[3,2-d]pyrimidine scaffold for the subsequent nucleophilic displacement reaction occurs in step (f) by preparing the corresponding 4-(1,2,4-triazolyl)-pyrido[3,2-d]pyrimidine derivative or 4-chloro-pyrido[3,2-d]pyrimidine derivative. The 4-triazolyl derivative can be obtained by treating the 4-oxo-pyrido[3,2-d]pyrimidine derivative with POCl₃ or 4-chlorophenyl phosphorodichloridate and 1,2,4-triazole in an appropriate solvent such as, but not limited to, pyridine or acetonitrile. The 4-chloro derivative can be obtained by treating the 4-oxo-pyrido[3,2-d]pyrimidine derivative with thionyl chloride or POCl₃. The chlorine atom or triazolyl group is designated as L in FIG. 1. Nucleophilic displacement of the triazolyl group or chlorine atom occurs in step (g) by reaction with an appropriate nucleophile represented by the structural formula R₂H, wherein R₂ is as defined in the structural formula (I), in a polar aprotic solvent. When piperazine is introduced in step (g) of this method, as well as in the corresponding step of some of the further methods described herein, the second nitrogen atom of this piperazin-1-yl substituent may, if desired, be coupled with a suitable carboxylic acid or thio-carboxylic acid chloride or sulfonyl chloride R₅Cl at room temperature in a solvent such as pyridine. Representative but non limiting examples of commercially available N-alkylpiperazines, N-arylpiperazines and N-alkylarylpiperazines that can suitably be used in step (g) of this method, as well as in the corresponding step of some of the further methods described herein, include 1-cyclohexylpiperazine, 1-cyclopentylpiperazine, 1-(2,6-dichlorobenzyl)piperazine, 1-(3,4-dichlorophenyl)piperazine, 1-[2-(dimethylamino)-ethyl]piperazine, 1-[3-(dimethyl-amino)propyl]piperazine, 1-(3,4-dimethylphenyl)piperazine, 1-(2-ethoxyethyl)piperazine, 1-isobutylpiperazine, 1-(1-methylpiperidin-4-yl-methyl)piperazine, 1-(2-nitro-4-trifluoromethylphenyl)piperazine, 1-(2-phenoxyethyl)piperazine, 1-(1-phenylethyl)piperazine, 2-(piperazin-1-yl)acetic acid ethyl ester, 2-(piperazin-1-yl)acetic acid N-methyl-N-phenyl amide, 2-(piperazin-1-yl)acetic acid N-(2-thiazolyl)amide, 2-[2-(piperazin-1-yl)ethyl]-1,3-dioxolan-3-(1-piperazinyl)propionitrile, 1-[(2-pyridyl)-methyl]piperazine and 1-thiazol-2-yl-piperazine. In the final step (h), the amino protecting group is cleaved off by using standard cleavage conditions such as acidic or basic hydrolysis.

FIG. 1 also relates to a synthetic pathway useful for obtaining 2,4,6-tri-substituted pyrido(3,2-d)pyrimidine derivatives represented by one of the formulae (II), (III) and (IV). Although their substituents R₂′ and/or R₃′ are not shown in the figure, the skilled person readily understands that the above-mentioned chemical methodologies are similarly able to provide these derivatives.

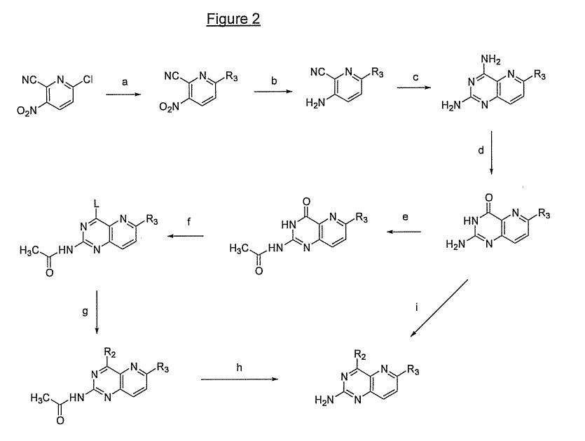

FIG. 2 schematically shows a second method for making 2,4,6-tri-substituted pyrido(3,2-d)pyrimidine derivatives represented by the structural formula (I) wherein the substituent in position 2 is amino, as well as intermediates therefor wherein the substituent in position 2 is a N-protected amino such as acetamido and/or wherein the substituent in position 4 is hydroxy, chloro or triazolyl. In step (a), 6-chloro-2-cyano-3-nitropyridine is subjected to a palladium-catalyzed reaction such as, but not limited to, a Suzuki reaction with an arylboronic or heteroarylboronic acid, or an ester thereof, to yield the corresponding biaryl derivative or a Heck reaction with a terminal alkene or alkyne leading to the formation of an alkenyl or alkynyl derivative. The 3-nitro group is reduced in step (b), either catalytically (e.g. by using platinum or palladium under an atmosphere of hydrogen) or chemically (e.g. by using iron or tin under acidic conditions). A ring closure reaction leading to the formation of the pyrido[3,4-d]pyrimidine scaffold occurs in step (c) by treatment of the 6-R₃-substituted-2-cyano-3-aminopyridine intermediate with a ring closure reagent such as, but not limited to, chloroformamidine or guanidine. Aqueous hydrolysis of the 4-amino group, either under acidic or alcaline conditions, yields the 2-amino-6-R₃-pyrido[3,2-d]pyrimidin-4(3H)one. In step (e), the amino group at position 2 is protected, for example by a pivaloyl (not shown in FIG. 2) or acetyl group, by reaction with acetic anhydride or pivaloyl anhydride respectively, in pyridine as a solvent, thus resulting into the introduction of a N-protected amino group at position 2 such as, but not limited to, acetamido or pivalamido. Activation of the tautomeric hydroxyl group at position 4 of the pyrido[3,2-d]pyrimidine scaffold for the subsequent nucleophilic displacement reaction occurs in step (f) by preparing the corresponding 4-(1,2,4-triazolyl)-pyrido[3,2-d]pyrimidine derivative or 4-chloro-pyrido[3,2-d]pyrimidine derivative. The 4-triazolyl derivative can be obtained by treating the 4-oxo-pyrido[3,2-d]pyrimidine derivative with POCl₃ or 4-chlorophenyl phosphorodichloridate and 1,2,4-triazole in an appropriate solvent such as, but not limited to, pyridine or acetonitrile. The 4-chloro derivative can be obtained by treating the 4-oxo-pyrido[3,2-d]pyrimidine derivative with thionyl chloride or POCl₃. The triazolyl group or chlorine atom is designated as L in FIG. 2. Nucleophilic displacement of the triazolyl group or chlorine atom occurs in step (g) by reaction with an appropriate nucleophile represented by the structural formula R₂H, wherein R₂ is as defined in the structural formula (I), in a polar aprotic solvent. In the final step (h), the amino protecting group is cleaved off by using standard cleavage conditions such as acidic or basic hydrolysis. Alternatively, an alkylamino, arylamino or alkylarylamino group R₂ can also be directly introduced, in step (i), at position 4 of the pyrido[3,2-d]pyrimidine scaffold by treatment of the 2-amino-6-R₃-substituted-pyrido[3,2-d]pyrimidine with an appropriate alkylamine, arylamine or alkylarylamine in the presence of a suitable amount of 1,1,1,3,3,3-hexamethyldisilazane as a reagent.

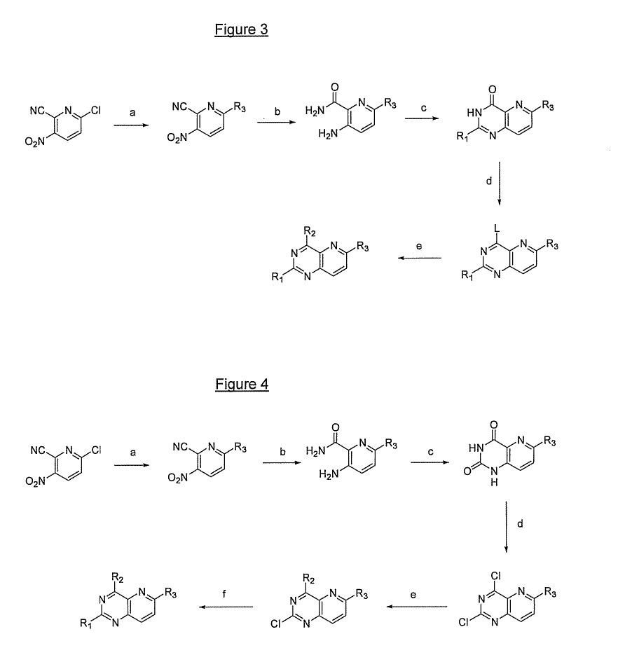

FIG. 2 also relates to a synthetic pathway useful for obtaining 2,4,6-tri-substituted pyrido(3,2-d)pyrimidine derivatives represented by one of the formulae (II), (III) and (IV). Although their substituents R₂′ and/or R₃′ are not shown in the figure, the skilled person readily understands that the above-mentioned chemical methodologies are similarly able to provide these derivatives. FIG. 3 schematically shows a method for making 2,4,6-tri-substituted pyrido(3,2-d)pyrimidine intermediates represented by the structural formula (I), as well as intermediates wherein the substituent in position 4 is hydroxy, chloro or triazolyl. In step (a), 6-chloro-2-cyano-3-nitropyridine is subjected to a palladium-catalyzed reaction such as, but not limited to, a Suzuki reaction with an arylboronic or heteroarylboronic acid, or an ester thereof, to yield the corresponding biaryl derivative or, alternatively, a Heck reaction with a terminal alkene or alkyne leading to the formation of alkenyl or alkynyl derivatives. In step (b), the 3-nitro group is reduced, either catalytically (e.g. by using platinum or palladium under an atmosphere of hydrogen) or chemically (e.g. by using iron or tin under acidic conditions) and at the same time the cyano group is hydrolyzed into a carboxamide function. Formation of the 2-R₁-substituted-pyrido[3,2-d]pyrimidine scaffold occurs in step (c) by treatment of a 6-R₃-substituted-2-carboxamido-3-aminopyridine derivative either with an orthoester (such as, but not limited to, triethyl orthoformate) or with an acid chloride followed by treatment with a base such as sodium hydroxide. Activation of the tautomeric hydroxyl group at position 4 of the pyrido[3,2-d]pyrimidine scaffold for the subsequent nucleophilic displacement reaction occurs in step (d) by preparing the corresponding 4-chloro-pyrido[3,2-d]pyrimidine derivative or the corresponding 4-(1,2,4-triazolyl)-pyrido[3,2-d]pyrimidine derivative. The triazolyl derivative can be obtained by treating the 4-oxo-pyrido[3,2-d]pyrimidine derivative with POCl₃ or 4-chlorophenyl phosphorodichloridate and 1,2,4-triazole in an appropriate solvent such as, but not limited to, pyridine or acetonitrile. The 4-chloro derivative can be obtained by treating the 4-oxo-pyrido[3,2-d]pyrimidine derivative with thionyl chloride or POCl₃. The triazolyl group or chlorine atom at position 4 are indicated as L in FIG. 3. Nucleophilic displacement of the chlorine atom or 1,2,4-triazolyl moiety occurs in step (e) by reaction with an appropriate nucleophile represented by the structural formula R₂H, wherein R₂ is as defined in the structural formula (I), in a polar protic or aprotic solvent.

FIG. 3 also relates to a synthetic pathway useful for obtaining 2,4,6-tri-substituted pyrido(3,2-d)pyrimidine derivatives represented by one of the formulae (II), (III) and (IV). Although their substituents R₂′ and/or R₃′ are not shown in the figure, the skilled person readily understands that the above-mentioned chemical methodologies are similarly able to provide these derivatives.

FIG. 4 schematically shows another method for making 2,4,6-tri-substituted pyrido(3,2-d)pyrimidine intermediates represented by the structural formula (I), as well as intermediates wherein the substituent in positions 2 and 4 are hydroxy or chloro. In step (a), 6-chloro-2-cyano-3-nitropyridine is subjected to a palladium-catalyzed reaction such as, but not limited to, a Suzuki reaction with an arylboronic or heteroarylboronic acid, or an ester thereof, to yield the corresponding biaryl derivative or, alternatively, a Heck reaction with a terminal alkene or alkyne leading to the formation of an alkenyl or alkynyl derivative. In step (b), the 3-nitro group is reduced, either catalytically (e.g. by using platinum or palladium under an atmosphere of hydrogen) or chemically (e.g. by using iron or tin under acidic conditions) and at the same time the cyano group is hydrolyzed into a carboxamide function. Ring closure reaction leading to the formation of the pyrido[3,2-d]pyrimidine scaffold occurs in step (c) by treatment of a 6-R₃-substituted-2-carboxamido-3-aminopyridine derivative either with a phosgene derivative in an aprotic solvent or with a carbonate (such as, but not limited to, dimethylcarbonate or diethylcarbonate) in a protic or aprotic solvent. Activation of the tautomeric hydroxyl groups at positions 2 and 4 of the pyrido[3,2-d]pyrimidine scaffold for the subsequent nucleophilic displacement reaction occurs in step (d) by preparing the corresponding 2,4-dichloro-pyrido[3,2-d]pyrimidine derivative, e.g. by treating the 4-oxo-pyrido[3,2-o]pyrimidine derivative with thionyl chloride or POCl₃. Selective nucleophilic displacement of the chlorine at position 4 occurs in step (e) by reaction with an appropriate nucleophile represented by the structural formula R₂H in a polar protic or aprotic solvent at an appropriate temperature. In step (f), the 2-chloro derivative is then treated with an appropriate nucleophile represented by the structural formula R₁H in a polar protic or aprotic solvent at an appropriate temperature in order to afford the desired 2,4,6-trisubstituted derivative.

FIG. 4 also relates to a synthetic pathway useful for obtaining 2,4,6-tri-substituted pyrido(3,2-d)pyrimidine derivatives represented by one of the formulae (II), (III) and (IV). Although their substituents R₂′ and/or R₃′ are not shown in the figure, the skilled person readily understands that the above-mentioned chemical methodologies are similarly able to provide these derivatives.

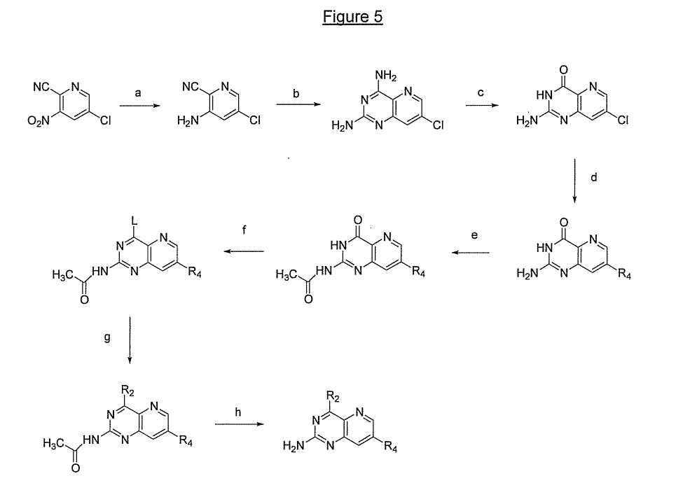

FIG. 5 schematically shows a first method for making 2,4,7-tri-substituted pyrido(3,2-d)pyrimidine derivatives represented by the structural formula (I) wherein the substituent in position 2 is amino, as well as intermediates therefor wherein the substituent in position 2 is a N-protected amino such as acetamido and/or wherein the substituent in position 4 is hydroxy, chloro or triazolyl. The nitro group of 5-chloro-2-cyano-3-nitropyridine is first reduced in step (a) either catalytically (e.g. by using platinum or palladium under an atmosphere of hydrogen) or chemically (e.g. by using iron or tin under acidic conditions). A ring closure reaction leading to the formation of the pyrido[3,2-d]pyrimidine scaffold occurs in step (b) by treatment of 5-chloro-2-cyano-3-aminopyridine with a ring closure reagent such as, but not limited to, chloroformamidine or guanidine. Aqueous hydrolysis under aqueous acidic conditions then yields 2-amino-7-chloro-pyrido[3,2-d]pyrimidin-4(3H)one in step (c). In step (d), the chlorine atom at position 7 can be used as a leaving group for a variety of palladium-catalyzed reactions such as, but not limited to, a Suzuki reaction (by treatment of 2-amino-7-chloro-pyrido[3,2-d]pyrimidin-4(3H)one with an arylboronic or heteroarylboronic acid, or an ester thereof, leading to the formation of a biaryl derivative) and a Heck reaction (by treatment of 2-amino-7-chloro-pyrido[3,2-d]pyrimidin-4(3H)one with a wide variety of terminal alkenes or alkynes, thus yielding alkenyl or alkynyl compounds). In step (e), the amino group at position 2 is protected, for example by a pivaloyl (not shown in FIG. 1) or acetyl group, by reaction with acetic anhydride or pivaloyl anhydride in pyridine as a solvent, thus resulting into the introduction of a N-protected amino group at position 2 such as, but not limited to, acetamido or pivalamido. Activation of the tautomeric hydroxyl group at position 4 of the pyrido[3,2-d]pyrimidine scaffold for the subsequent nucleophilic displacement reaction occurs in step (f) by preparing the corresponding 4-(1,2,4-triazolyl)-pyrido[3,2-d]pyrimidine derivative or 4-chloro-pyrido[3,2-d]pyrimidine derivative. The 4-triazolyl derivative can be obtained by treating the 4-oxo-pyrido[3,2-d]pyrimidine derivative with POCl₃ or 4-chlorophenyl phosphorodichloridate and 1,2,4-triazole in an appropriate solvent such as, but not limited to, pyridine or acetonitrile. The 4-chloro derivative can be obtained by treating the 4-oxo-pyrido[3,2-d]pyrimidine derivative with thionyl chloride or POCl₃. The chlorine atom or triazolyl group is designated as L in FIG. 5. Nucleophilic displacement of the triazolyl group or chlorine atom occurs in step (g) by reaction with an appropriate nucleophile represented by the structural formula R₂H, wherein R₂ is as defined in the structural formula (I), in a polar aprotic solvent. In the final step (h), the amino protecting group is cleaved off by using standard cleavage conditions such as acidic or basic hydrolysis.

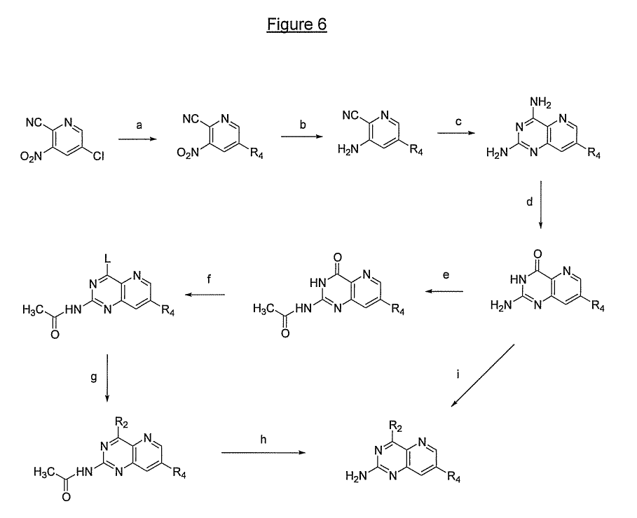

FIG. 6 schematically shows a second method for making 2,4,7-tri-substituted pyrido(3,2-d)pyrimidine derivatives represented by the structural formula (I) wherein the substituent in position 2 is amino, as well as intermediates therefor wherein the substituent in position 2 is a N-protected amino such as acetamido and/or wherein the substituent in position 4 is hydroxy, chloro or triazolyl. In step (a), 5-chloro-2-cyano-3-nitropyridine is subjected to a palladium-catalyzed reaction such as, but not limited to, a Suzuki reaction with an arylboronic or heteroarylboronic acid, or an ester thereof, to yield the corresponding biaryl derivative or a Heck reaction with a terminal alkene or alkyne leading to the formation of an alkenyl or alkynyl derivative. The 3-nitro group is reduced in step (b), either catalytically (e.g. by using platinum or palladium under an atmosphere of hydrogen) or chemically (e.g. by using iron or tin under acidic conditions). A ring closure reaction leading to the formation of the pyrido[3,4-d]pyrimidine scaffold occurs in step (c) by treatment of the 5-R₄-substituted-2-cyano-3-aminopyridine intermediate with a ring closure reagent such as, but not limited to, chloroformamidine or guanidine. Aqueous hydrolysis of the 4-amino group, either under acidic or alcaline conditions, yields the 2-amino-7-R₄-pyrido[3,2-d]pyrimidin-4(3H)one. In step (e), the amino group at position 2 is protected, for example by a pivaloyl (not shown in FIG. 2) or acetyl group, by reaction with acetic anhydride or pivaloyl anhydride respectively, in pyridine as a solvent, thus resulting into the introduction of a N-protected amino group at position 2 such as, but not limited to, acetamido or pivalamido. Activation of the tautomeric hydroxyl group at position 4 of the pyrido[3,2-d]pyrimidine scaffold for the subsequent nucleophilic displacement reaction occurs in step (f) by preparing the corresponding 4-(1,2,4-triazolyl)-pyrido[3,2-d]pyrimidine derivative or 4-chloro-pyrido[3,2-d]pyrimidine derivative. The 4-triazolyl derivative can be obtained by treating the 4-oxo-pyrido[3,2-d]pyrimidine derivative with POCl₃ or 4-chlorophenyl phosphorodichloridate and 1,2,4-triazole in an appropriate solvent such as, but not limited to, pyridine or acetonitrile. The 4-chloro derivative can be obtained by treating the 4-oxo-pyrido[3,2-d]pyrimidine derivative with thionyl chloride or POCl₃. The triazolyl group or chlorine atom is designated as L in FIG. 6. Nucleophilic displacement of the triazolyl group or chlorine atom occurs in step (g) by reaction with an appropriate nucleophile represented by the structural formula R₂H, wherein R₂ is as defined in the structural formula (I), in a polar aprotic solvent. In the final step (h), the amino protecting group is cleaved off by using standard cleavage conditions such as acidic or basic hydrolysis. Alternatively, an alkylamino, arylamino or alkylarylamino group R₂ can also be directly introduced, in step (i), at position 4 of the pyrido[3,2-d]pyrimidine scaffold by treatment of the 2-amino-7-R₄-substituted-pyrido[3,2-d]pyrimidine with an appropriate alkylamine, arylamine or alkylarylamine in the presence of a suitable amount of 1,1,1,3,3,3-hexamethyldisilazane as a reagent.

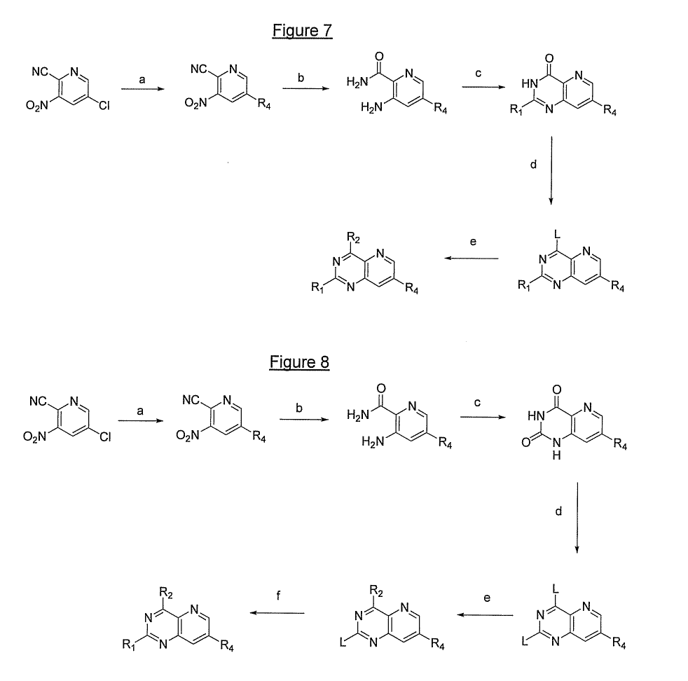

FIG. 7 schematically shows a method for making 2,4,7-tri-substituted pyrido(3,2-d)pyrimidine intermediates represented by the structural formula (I), as well as intermediates wherein the substituent in position 4 is hydroxy, chloro or triazolyl. In step (a), 5-chloro-2-cyano-3-nitropyridine is subjected to a palladium-catalyzed reaction such as, but not limited to, a Suzuki reaction with an arylboronic or heteroarylboronic acid, or an ester thereof, to yield the corresponding biaryl derivative or, alternatively, a Heck reaction with a terminal alkene or alkyne leading to the formation of alkenyl or alkynyl derivatives. In step (b), the 3-nitro group is reduced, either catalytically (e.g. by using platinum or palladium under an atmosphere of hydrogen) or chemically (e.g. by using iron or tin under acidic conditions) and at the same time the cyano group is hydrolyzed into a carboxamide function. Formation of the 2-R₁-substituted-pyrido[3,2-d]pyrimidine scaffold occurs in step (c) by treatment of a 5-R₄-substituted-2-carboxamido-3-aminopyridine derivative either with an orthoester (such as, but not limited to, triethyl orthoformate) or with an acid chloride followed by treatment with a base such as sodium hydroxide. Activation of the tautomeric hydroxyl group at position 4 of the pyrido[3,2-d]pyrimidine scaffold for the subsequent nucleophilic displacement reaction occurs in step (d) by preparing the corresponding 4-chloro-pyrido[3,2-d]pyrimidine derivative or the corresponding 4-(1,2,4-triazolyl)-pyrido[3,2-d]pyrimidine derivative. The triazolyl derivative can be obtained by treating the 4-oxo-pyrido[3,2-d]pyrimidine derivative with POCl₃ or 4-chlorophenyl phosphorodichloridate and 1,2,4-triazole in an appropriate solvent such as, but not limited to, pyridine or acetonitrile. The 4-chloro derivative can be obtained by treating the 4-oxo-pyrido[3,2-d]pyrimidine derivative with thionyl chloride or POCl₃. The triazolyl group or chlorine atom at position 4 are indicated as L in FIG. 7. Nucleophilic displacement of the chlorine atom or 1,2,4-triazolyl moiety occurs in step (e) by reaction with an appropriate nucleophile represented by the structural formula R₂H, wherein R₂ is as defined in the structural formula (I), in a polar protic or aprotic solvent.

FIG. 8 schematically shows another method for making 2,4,7-tri-substituted pyrido(3,2-d)pyrimidine intermediates represented by the structural formula (I), as well as intermediates wherein the substituent in positions 2 and 4 are hydroxy or chloro. In step (a), 5-chloro-2-cyano-3-nitropyridine is subjected to a palladium-catalyzed reaction such as, but not limited to, a Suzuki reaction with an arylboronic or heteroarylboronic acid, or an ester thereof, to yield the corresponding biaryl derivative or, alternatively, a Heck reaction with a terminal alkene or alkyne leading to the formation of an alkenyl or alkynyl derivative. In step (b), the 3-nitro group is reduced, either catalytically (e.g. by using platinum or palladium under an atmosphere of hydrogen) or chemically (e.g. by using iron or tin under acidic conditions) and at the same time the cyano group is hydrolyzed into a carboxamide function. Ring closure reaction leading to the formation of the pyrido[3,2-d]pyrimidine scaffold occurs in step (c) by treatment of a 5-R₄-substituted-2-carboxamido-3-aminopyridine derivative either with a phosgene derivative in an aprotic solvent or with a carbonate (such as, but not limited to, dimethylcarbonate or diethylcarbonate) in a protic or aprotic solvent. Activation of the tautomeric hydroxyl groups at positions 2 and 4 of the pyrido[3,2-d]pyrimidine scaffold for the subsequent nucleophilic displacement reaction occurs in step (d) by preparing the corresponding 2,4-dichloro-pyrido[3,2-d]pyrimidine derivative, e.g. by treating the 4-oxo-pyrido[3,2-d]pyrimidine derivative with thionyl chloride or POCl₃. Selective nucleophilic displacement of the chlorine at position 4 occurs in step (e) by reaction with an appropriate nucleophile represented by the structural formula R₂H in a polar protic or aprotic solvent at an appropriate temperature. In step (f), the 2-chloro derivative is then treated with an appropriate nucleophile represented by the structural formula R₁H in a polar protic or aprotic solvent at an appropriate temperature in order to afford the desired 2,4,7-trisubstituted derivative.

The methods described in relation to FIGS. 1 to 8 make use of an arylboronic or heteroarylboronic acid, or e.g. a pinacol ester thereof, for introducing a substituent onto the core structure. In these methods, suitable aryl-boronic acids include, but are not limited to, the following commercially available materials wherein the aryl group is 3-acetamidophenyl, 4-acetamindophenyl, 4-acetylphenyl, 3-acetylphenyl, 2-acetylphenyl, 5-acetyl-2-chlorophenyl, 4-acetyl-3-fluorophenyl, 5-acetyl-2-fluorophenyl, 3-aminophenyl, 4-aminomethylphenyl, 3-aminophenyl, 4-benzyloxybenzene, 3-benzyloxybenzene, 4-benzyloxy-2-fluorophenyl, 4-benzyloxy-3-fluorophenyl, biphenyl-3-, 3,5-bis(trifluoromethyl)benzene, 4-bromophenyl, 3-bromophenyl, 4-bromo-2,5-dimethylphenyl, 2-bromo-5-fluorophenyl, 2-Bromo-6-fluorophenyl, 4-carboxyphenyl, 2-carboxyphenyl, 2-carboxy-5-fluorophenyl, 4-carboxy-2-chlorophenyl, 5-carboxy-2-chlorophenyl, 4-carboxy-3-chlorophenyl, 3-carboxyphenyl, 2-chloro-5-formylphenyl, 2-chloro-5-hydroxyphenyl, 3-chloro-4-fluorophenyl, 2-chloro-4-fluorophenyl, 4-chloro-2-fluorophenyl, 3-chloro-5-methoxyphenyl, 2-chloro-4-methylphenyl, 2-chloro-5-methylphenyl, 2-chloro-5-trifluoromethoxyphenyl, 3-chloro-5-trifluoromethylphenyl, 4-chloro-2-trifluoromethylphenyl, 4-chlorophenyl, 3-chlorophenyl, 2-chlorophenyl, 4-cyanophenyl, 3-cyanophenyl, 2-cyanophenyl, 3,5-dibromophenyl, 2,6-dichlorophenyl, 3,4-dichlorophenyl, 2,4-dichlorophenyl, 2,3-dichlorophenyl, 3,5-dichlorophenyl, 3,5-difluorophenyl, 3,5-difluoro-2-methoxyphenyl, 3,4-difluorophenyl, 2,6-difluorophenyl, 2,5-difluorophenyl, 2,4-difluorophenyl, 2,3-difluorophenyl, 2,3-dihydro-1,4-benzodioin-6-yl, 2,4-dimethoxybenzene, 4-(N,N-dimethylamino)phenyl, 2-(N,N-dimethylaminomethyl)phenyl, 3,5-dimethylphenyl, 3,4-dimethylphenyl, 2,6-dimethylphenyl, 2,6-dimethoxyphenyl, 2,5-dimethoxyphenyl, 2,4-dimethoxyphenyl, 4-ethoxyphenyl, 2-ethoxyphenyl, 4-ethoxycarbonylphenyl, 3-ethoxycarbonylphenyl, 4-ethylphenyl, 4-fluorophenyl, 3-fluorophenyl, 2-fluorophenyl, 3-fluoro-4-formylphenyl, 4-fluoro-2-methylphenyl, 2-fluoro-5-methylphenyl, 4-fluoro-3-formylphenyl, 2-fluoro-5-methoxyphenyl, 5-fluoro-2-methoxycarbonylphenyl, 2-formyl-5-methoxyphenyl, 5-formyl-2-methoxyphenyl, 2-formyl-5-methylphenyl, 4-formylphenyl, 3-formylphenyl, 2-formylphenyl, 3-hydroxy-4-methoxycarbonylphenyl, 4-(hydroxymethyl)phenyl, 3-(hydroxymethyl)phenyl, 4-hydroxyphenyl, 3-hydroxyphenyl, 4-iodophenyl, 3-iodophenyl, 3-isopropoxycarbonylphenyl, 4-isopropoxycarbonylphenyl, 4-methanesulfonylphenyl, 2-methoxy-5-formylphenyl, 5-methoxy-2-formylphenyl, 4-methoxy-2-formylphenyl, 4-methoxycarbonylphenyl, 3-methoxycarbonylphenyl, 4-methoxyphenyl, 3-methoxyphenyl, 2-methoxyphenyl, 3,4-methylenedioxyphenyl, 4-methylphenyl, 2-methylphenyl, 4-(methylthio)phenyl, 3-(methylthio)phenyl, 4-morpholinophenyl, 3-nitrophenyl, 4-phenoxyphenyl, 4-(tert-butoxycarbonylamino)-3-methoxyphenyl, 2-(tert-butoxycarbonyl)phenyl, 3-(tert-butoxycarbonyl)phenyl, 4-(tert-butoxycarbonyl)phenyl, 4-tert-butylphenyl, 4-(tetrahdro-2H-pyran-2-yloxy)phenyl, 4-(2-thienyl)phenyl, trans-β-styrene, 4-tolyl, 3-tolyl, 2-tolyl, 4-trifluoromethoxyphenyl, 4-(trimethylammonium)methylphenyl, 2,4,6-trimethylphenyl, 3,4,5-trifluorophenyl, 3-trifluoromethylphenyl, 4-trifluoromethoxyphenyl, 3-trifluoromethoxyphenyl, 3-trifluoromethylphenyl, 2-trifluoromethylphenyl, 3,4,5-trimethoxyphenyl, 4-vinylphenyl, 6-benzyloxy-2-naphthyl, 1-naphthalene, 2-naphthalene, or 1-biphenylenyl.

In these methods, suitable heterocyclic-boronic acids include, but are not limited to, the following commercially available materials wherein the heterocyclic group is 2-acetamidopyridin-5-yl, 2-benzothienyl, 1-benzothiophen-3-yl, 1-benzothiophen-2-yl, 2-bromo-3-chloropyridin-4-yl, 5-bromo-2,3-dihydrobenzo[b]furan-7-yl, 2-bromo-3-methylpyridin-5-yl, 2-bromopyridin-5-yl, 5-bromothien-2-yl, 2-chloro-6-isopropylpyridin-3-yl, 2-chloro-3-methylpyridin-5-yl, 5-chlorothien-2-yl, dibenzo[b,d]furan-4-yl, 2-chloro-3-fluoropyridin-4-yl, dibenzo[b,d]thien-4-yl, 3,4-dihydro-2H-1,5-benzodioxepin-7-yl, 2,5-dibromo-3-pyridinyl, 2,6-dichloro-pyridin-3-yl, 2,3-dihydro-1-benzofuran-5-yl, 2,4-dimethoxypyrimidin-5-yl, 3,5-dimethylisoxazol-4-yl, 1-[1,3]dioxolan-2-ylmethyl-4-1H-pyrazolyl, 2,4-dioxo-1,2,34-tetrahydro-5-pyrimidinyl, 2,4-di(tert-butoxy)pyrimidin-5-yl, 2-ethoxypyridin-3-yl, 2-fluoro-3-methylpyridin-5-yl, 2-fluoropyridin-3-yl, 2-fluoropyridin-5-yl, 5-formyl-2-furyl, 5-formylthiophen-2-yl, furan-3-yl, furan-2-yl, 5-indolyl, isoquinolin-4-yl, 2-methoxypyrimidin-5-yl, 5-methyl-1-benzothiophen-2-yl, 5-methylfuran-2-yl, 5-methyl-3-phenyl-4-isoxazolyl, 5-(methylsulfanyl)-2-thienyl, 3-methyl-pyridin-2-yl, (5-methyl)thien-2-yl, 5-methylpyridin-2-yl, 5-methylpyridin-3-yl, 2-methoxypyridine-3-yl, (4-methyl)thien-2-yl, 2-methoxypyridin-5-yl, 1-(phenylsulfonyl)-1H-indol-3-yl, 1-(phenylsulfonyl)-1H-indol-3-yl, 5-phenyl-2-thienyl, pyridin-4-yl, pyridin-3-yl, 5-pyrimidinyl, 4-phenoxathiinyl, 8-quinolinyl, 3-quinolinyl, 1-tert-butoxycarbonyl-2-pyrrolyl, 1-(tert-butoxycarbonyl)-5-bromo-1H-indol-2-yl, 1-(tert-butoxycarbonyl)-1H-indol-2-yl, 1-(tert-butoxycarbonyl)-5-methoxy-1H-indol-2-yl, 1-thianthrenyl-3-thienyl, or 2-thienyl.

Also, as shown in certain examples below, when position 4 of the core structure is substituted with a heteroaryl group (e.g. piperazinyl or pyrrolidinyl) itself substituted with a carbamoyl or thiocarbamoyl group, a relevant method of synthesis includes a reaction step with an isocyanate or an isothiocyanate. Aryl isocyanates suitable for use in such a synthesis include, but are not limited to, 4-fluorophenyl isocyanate, phenyl isocyanate, m-tolyl isocyanate, p-tolyl isocyanate, 4-chlorophenyl isocyanate, ethyl 4-isocyanatobenzoate, 2-fluoro-phenyl isocyanate, 3-fluorophenyl isocyanate, α,α,α-trifluoro-o-tolyl isocyanate, tolylene-2,4-diisocyanate, tolylene 2,6-diisocyanate, 4-methoxyphenyl isocyanate, 4-bromophenyl isocyanate, 2-methoxyphenyl isocyanate, 3-Methoxyphenyl isocyanate, 2,4-dichlorophenyl isocyanate, o-tolyl isocyanate, 3,4-dichlorophenyl isocyanate, 2-nitrophenyl isocyanate, 4-nitrophenyl isocyanate, 2,4-difluorophenyl isocyanate, 2-bromophenyl isocyanate, 2,6-difluoro-phenyl isocyanate, 2-(trifluoromethoxy)phenyl isocyanate, 2-chloro-5-(trifluoro-methyl)phenyl isocyanate, 4-chloro-2-(trifluoro-methyl)phenyl isocyanate, 4-chloro-3-(trifluoromethyl)phenyl isocyanate, 2,5-difluoro-phenyl isocyanate, 4-(trifluoro-methoxy)phenyl isocyanate, 2-ethoxyphenyl isocyanate, 4-ethoxyphenyl isocyanate, 4-isopropylphenyl isocyanate, 3-acetylphenyl isocyanate, 2,6-diisopropylphenyl isocyanate, 3-bromophenyl isocyanate, 3,5-dichlorophenyl isocyanate, 4-fluoro-3-nitrophenyl isocyanate, 3,5-dimethylphenyl isocyanate, 3,5-bis(trifluoromethyl)phenyl isocyanate, 3-cyanophenyl isocyanate, 4-(methylthio)phenyl isocyanate, 2-ethylphenyl isocyanate, 2,6-dimethyl-phenyl isocyanate, α,α,α-trifluoro-p-tolyl isocyanate, 2,3-dichlorophenyl isocyanate, 4-methyl-3-nitrophenyl isocyanate, 2,4-dimethoxyphenyl isocyanate, 4-(chloro-methyl)phenyl isocyanate, 4-bromo-2-chlorophenyl isocyanate, 2-bromo-4,6-difluoro-phenyl isocyanate, 4-bromo-2-fluoro-phenyl isocyanate, 4-(dimethylamino)phenyl isocyanate, 2-fluoro-5-methylphenyl isocyanate, 4-fluoro-2-nitrophenyl isocyanate, 2-fluoro-3-(trifluoromethyl)phenyl isocyanate, 2-fluoro-5-(trifluoromethyl)phenyl isocyanate, 2-fluoro-6-(trifluoromethyl)-phenyl isocyanate, 4-fluoro-2-(trifluoromethyl)phenyl isocyanate, 4-fluoro-3-(trifluoromethyl)phenyl isocyanate, 4-(heptyloxy)phenyl isocyanate, 2-iodophenyl isocyanate, 2-naphthyl isocyanate, 2-n-propylphenyl isocyanate, 4-(trifluoromethyl-thio)phenyl isocyanate, 2,3,4-trifluorophenyl isocyanate, 2,6-dichlorophenyl isocyanate, 3-nitrophenyl isocyanate, 3-chlorophenyl isocyanate, 2-chlorophenyl isocyanate, 1-naphthyl isocyanate, 2,3-dimethylphenyl isocyanate, 3-chloro-4-fluorophenyl isocyanate, 2,5-dimethylphenyl isocyanate, 3,4-difluorophenyl isocyanate, 2,3-dihydro-1-benzofuran-5-yl isocyanate, 2,3-dihydro-1,4-benzodioxin-6-yl isocyanate, 6-fluoro-4H-1,3-benzodioxin-8-yl isocyanate, 2,1,3-benzothiadiazol-4-yl isocyanate, 3,4-dihydro-2H-1,5-benzodioxepin-7-yl isocyanate, 3-(cyclopentyloxy)-4-methoxyphenyl isocyanate, 2-(methylthio)phenyl isocyanate, 2-(tert-butyl)phenyl isocyanate, 4-(tert-butyl)phenyl isocyanate, 3-chloro-2-methylphenyl isocyanate, 4-butyl-2-methylphenyl isocyanate, 2-ethyl-6-methylphenyl isocyanate, 4-chloro-3-nitrophenyl isocyanate, 4-bromo-2-methylphenyl isocyanate, 3-(methylthio)phenyl isocyanate, 5,5,8,8-tetramethyl-5,6,7,8-tetrahydro-2-naphthalenyl isocyanate, 5-fluoro-2-methylphenyl isocyanate, 4-phenoxyphenyl isocyanate, 4-methoxy-2-methyl-phenyl isocyanate, α,α,α-trifluoro-m-tolyl isocyanate, 2,6-dibromo-4-isopropylphenyl isocyanate, 2,6-dimethoxyphenyl isocyanate, 2-(4-isocyanatophenyl)thiophene, 4-(3-isocyanatophenyl)-2-methyl-1,3-thiazole, 3-(3-isocyanatophenyl)-5-methyl-1,2,4-oxa-diazole, 1-benzothiophen-5-yl isocyanate, 1-(3-isocyanatophenyl)-1H-pyrrole, 1-(4-isocyanatophenyl)-1H-pyrrole, 3,5-dimethoxyphenyl isocyanate and 2,4,6-trichloro-phenyl isocyanate.

Aryl isothiocyanates suitable for use in such a synthesis include, but are not limited to, phenyl isothiocyanate, 4-fluorophenyl isothiocyanate, methyl 2-isocyanatobenzoate, 2-chlorophenyl isothiocyanate, 3-chlorophenyl isothiocyanate, otolyl isothiocyanate, m-tolyl isothiocyanate, p-tolyl isothiocyanate, 2-methoxyphenyl isothiocyanate, 2-bromophenyl isothiocyanate, 3-bromophenyl isothiocyanate, 2,4-dichloro-phenyl isothiocyanate, 2-fluoro phenylisothiocyanate, 4-methoxyphenyl isothiocyanate, α,α,α-trifluoro-m-tolyl isothiocyanate, 3-fluorophenyl isothiocyanate, 3,5-bis(trifluoromethyl)phenyl isothiocyanate, 1-naphthyl isothiocyanate, 4-dimethylamino-1-naphthyl isothiocyanate, 4-(methylthio)phenyl isothiocyanate, 2-methoxy-5-methylphenyl isothiocyanate, 4-cyanophenyl isothiocyanate, 3-chloro-4-fluorophenyl isothiocyanate, 4-(trifluoromethoxy)phenyl isothiocyanate, 3,5-dimethylphenyl isothiocyanate, 3,5-dimethoxyphenyl isothiocyanate, 4-chlorophenyl isothiocyanate, 3,4-dimethoxyphenyl isothiocyanate, 2,6-dimethylphenyl isothiocyanate, 3-methoxyphenyl isothiocyanate, mesityl isothiocyanate, 4-(benzyloxy)phenyl isothiocyanate, 2,4-dimethylphenyl isothiocyanate, 2-bromo-5-fluorophenyl isothiocyanate, 5-fluoro-2-methylphenyl isothiocyanate, 4-chloro-2,5-dimethoxyphenyl isothiocyanate, 2,5-dichlorophenyl isothiocyanate, 2-(tert-butyl)-4,5,6-trimethyl-3-nitrophenyl isothiocyanate, 2-isopropyl-6-methylphenyl isothiocyanate, 4-ethoxyphenyl isothiocyanate, 5-chloro-2-methylphenyl isothiocyanate, 2-ethyl-6-methylphenyl isothiocyanate and 4-(trifluoromethyl)phenyl isothiocyanate, 4-nitrophenyl isothiocyanate, 4-bromophenyl isothiocyanate, 2,3-dihydro-1,4-benzodioxin-6-yl isothiocyanate, 1,3-benzodioxol-5-yl isothiocyanate, 4-(1H-pyrazol-1-yl)phenyl isothiocyanate, 2-(trifluoromethyl)phenyl isothiocyanate, 2,3-dimethylphenyl isothiocyanate, 2-isopropyl phenyl isothiocyanate, 4-iso-propylphenyl isothiocyanate, 5-chloro-2-methoxyphenyl isothiocyanate, 2,4-dimethoxyphenyl isothiocyanate, 2,4-dichloro-6-methylphenyl isothiocyanate, 2-bromo-4-isopropylphenyl isothiocyanate, and 5-chloro-2-fluorophenyl isothiocyanate.

Alkyl isocyanates and alkyl isothiocyanates may also be useful in such a synthesis, depending upon the type of carbamoyl group to be introduced onto the heteroaryl group on position 4 of the core structure.

In another particular embodiment, the invention relates to a group of pyrido(3,2-d)pyrimidine derivatives, as well as pharmaceutical compositions comprising such pyrido(3,2-d)pyrimidine derivatives as active principle, represented by one of the above structural formulae (I), (II), (III) and (IV) and being in the form of a pharmaceutically acceptable salt. The latter include any therapeutically active non-toxic addition salt which compounds represented by one of the structural formulae (I), (II), (III) and (IV) are able to form with a salt-forming agent. Such addition salts may conveniently be obtained by treating the pyrido(3,2-d)pyrimidine derivatives of the invention with an appropriate salt-forming acid or base. For instance, pyrido(3,2-d)pyrimidine derivatives having basic properties may be converted into the corresponding therapeutically active, non-toxic acid addition salt form by treating the free base form with a suitable amount of an appropiate acid following conventional procedures. Examples of such appropriate salt-forming acids include, for instance, inorganic acids resulting in forming salts such as but not limited to hydrohalides (e.g. hydrochloride and hydrobromide), sulfate, nitrate, phosphate, diphosphate, carbonate, bicarbonate, and the like; and organic monocarboxylic or dicarboxylic acids resulting in forming salts such as, for example, acetate, propanoate, hydroxyacetate, 2-hydroxypropanoate, 2-oxopropanoate, lactate, pyruvate, oxalate, malonate, succinate, maleate, fumarate, malate, tartrate, citrate, methanesulfonate, ethanesulfonate, benzoate, 2-hydroxybenzoate, 4-amino-2-hydroxybenzoate, benzene-sulfonate, p-toluenesulfonate, salicylate, p-aminosalicylate, pamoate, bitartrate, camphorsulfonate, edetate, 1,2-ethanedisulfonate, fumarate, glucoheptonate, gluconate, glutamate, hexylresorcinate, hydroxynaphtoate, hydroxyethanesulfonate, mandelate, methylsulfate, pantothenate, stearate, as well as salts derived from ethanedioic, propanedioic, butanedioic, (Z)-2-butenedioic, (E) 2-butenedioic, 2-hydroxybutanedioic, 2,3-dihydroxybutane-dioic, 2-hydroxy-1,2,3-propanetricarboxylic and cyclohexanesulfamic acids and the like. Pyrido(3,2-d)pyrimidine derivatives represented by one of the structural formulae (I), (II), (III) and (IV) having acidic properties may be converted in a similar manner into the corresponding therapeutically active, non-toxic base addition salt form. Examples of appropriate salt-forming bases include, for instance, inorganic bases like metallic hydroxides such as but not limited to those of alkali and alkaline-earth metals like calcium, lithium, magnesium, potassium and sodium, or zinc, resulting in the corresponding metal salt; organic bases such as but not limited to ammonia, alkylamines, benzathine, hydrabamine, arginine, lysine, N,N′-dibenzylethylenediamine, chloroprocaine, choline, diethanolamine, ethylene-diamine, N-methylglucamine, procaine and the like.

Reaction conditions for treating the pyrido(3,2-d)pyrimidine derivatives represented by one of the structural formulae (I), (II), (III) and (IV) of this invention with an appropriate salt-forming acid or base are similar to standard conditions involving the same acid or base but different organic compounds with basic or acidic properties, respectively. Preferably, in view of its use in a pharmaceutical composition or in the manufacture of a medicament for treating specific diseases, the pharmaceutically acceptable salt will be designed, i.e. the salt-forming acid or base will be selected so as to impart greater water-solubility, lower toxicity, greater stability and/or slower dissolution rate to the pyrido(3,2-d)pyrimidine derivative of this invention.

The present invention further provides the use of a pyrido(3,2-d)pyrimidine derivative represented by the structural formula (I), or a pharmaceutically acceptable salt or a solvate thereof, as a biologically-active ingredient, i.e. active principle, especially as a medicine or a diagnostic agent or for the manufacture of a medicament or a diagnostic kit. In particular the said medicament may be for the prevention or treatment of a pathologic condition selected from the group consisting of:
immune disorders, in particular organ and cells transplant rejections, and autoimmune disorders,
cardiovascular disorders,
disorders of the central nervous system,
TNF-α-related disorders,
viral diseases,
disorders mediated by phosphodiesterase-4 activity, and
cell proliferative disorders.

The present invention further provides the use of a pyrido(3,2-d)pyrimidine derivative represented by one of the structural formulae (II), (III) and (IV) or a pharmaceutically acceptable salt or a solvate thereof, as a biologically-active ingredient, i.e. active principle, especially as a medicine or for the manufacture of a medicament for treating an immune disorder or for preventing a transplant rejection. The pathologic conditions and disorders concerned by the said use, and the corresponding methods of prevention or treatment, are detailed hereinbelow. Any of the uses mentioned with respect to the present invention may be restricted to a non-medical use (e.g. in a cosmetic composition), a non-therapeutic use, a non-diagnostic use, a non-human use (e.g. in a veterinary composition), or exclusively an in-vitro use, or a use with cells remote from an animal.

The invention further relates to a pharmaceutical composition comprising:
(a) one or more pyrido(3,2-d)pyrimidine derivatives represented by one of the structural formulae (I), (II), (III) and (IV), and
(b) one or more pharmaceutically acceptable carriers.

In another embodiment, this invention provides combinations, preferably synergistic combinations, of one or more pyrido(3,2-d)pyrimidine derivatives represented by one of the structural formulae (I), (II), (III) and (IV), with one or more biologically-active drugs being preferably selected from the group consisting of immunosuppressant and/or immunomodulator drugs, antineoplastic drugs, and antiviral agents. As is conventional in the art, the evaluation of a synergistic effect in a drug combination may be made by analyzing the quantification of the interactions between individual drugs, using the median effect principle described by Chou et al. in Adv. Enzyme Reg. (1984) 22:27. Briefly, this principle states that interactions (synergism, additivity, antagonism) between two drugs can be quantified using the combination index (hereinafter referred as CI) defined by the following equation:

$${CI}_{x} = \frac{{ED}_{x}^{1 c}}{{ED}_{x}^{1 a}} + \frac{{ED}_{x}^{2 c}}{{ED}_{x}^{2 a}}$$

wherein EDₓ is the dose of the first or respectively second drug used alone (1a, 2a), or in combination with the second or respectively first drug (1c, 2c), which is needed to produce a given effect. The said first and second drug have synergistic or additive or antagonistic effects depending upon CI<1, CI=1, or CI>1, respectively. As will be explained in more detail herein below, this principle may be applied to a number of desirable effects such as, but not limited to, an activity against transplant rejection, an activity against immunosuppression or immunomodulation, or an activity against cell proliferation.

For instance the present invention relates to a pharmaceutical composition or combined preparation having synergistic effects against immuno-suppression or immunomodulation and containing:
(a) one or more immunosuppressant and/or immunomodulator drugs, and
(b) at least one pyrido(3,2-d)pyrimidine derivative represented by one of the structural formulae (I), (II), (III) and (IV), and
(c) optionally one or more pharmaceutical excipients or pharmaceutically acceptable carriers,
   for simultaneous, separate or sequential use in the treatment or prevention of autoimmune disorders and/or in transplant-rejections.

Suitable immunosuppressant drugs for inclusion in the synergistic compo-sitions or combined preparations of this invention belong to a well known therapeutic class. They are preferably selected from the group consisting of cyclosporin A, substituted xanthines (e.g. methylxanthines such as pentoxyfylline), daltroban, sirolimus, tacrolimus, rapamycin (and derivatives thereof such as defined below), leflunomide (or its main active metabolite A771726, or analogs thereof called malononitrilamides), mycophenolic acid and salts thereof (including the sodium salt marketed under the trade name Mofetil®), adrenocortical steroids, azathioprine, brequinar, gusperimus, 6-mercaptopurine, mizoribine, chloroquine, hydroxy-chloroquine and monoclonal antibodies with immunosuppressive properties (e.g. etanercept, infliximab or kineret). Adrenocortical steroids within the meaning of this invention mainly include glucocorticoids such as but not limited to ciprocinonide, desoxycorticisterone, fludrocortisone, flumoxonide, hydrocortisone, naflocort, procinonide, timobesone, tipredane, dexamethasone, methylprednisolone, methotrexate, prednisone, prednisolone, triamcinolone and pharmaceutically acceptable salts thereof. Rapamycin derivatives as referred herein include O-alkylated derivatives, particularly 9-deoxorapamycins, 26-dihydrorapamycins, 40-O-substituted rapamycins and 28,40-O,O-disubstituted rapamycins (as disclosed in U.S. Pat. No. 5,665,772) such as 40-O-(2-hydroxy)ethyl rapamycin—also known as SDZ-RAD-, pegylated rapamycin (as disclosed in U.S. Pat. No. 5,780,462), ethers of 7-desmethylrapamycin (as disclosed in U.S. Pat. No. 6,440,991) and polyethylene glycol esters of SDZ-RAD (as disclosed in U.S. Pat. No. 6,331,547).

Suitable immunomodulator drugs for inclusion into the synergistic immunomodulating pharmaceutical compositions or combined preparations of this invention are preferably selected from the group consisting of acemannan, amiprilose, bucillamine, dimepranol, ditiocarb sodium, imiquimod, Inosine Pranobex, interferon-β, interferon-γ, lentinan, levamisole, lisophylline, pidotimod, romurtide, platonin, procodazole, propagermanium, thymomodulin, thymopentin and ubenimex.

Synergistic activity of the pharmaceutical compositions or combined preparations of this invention against immunosuppression or immuno-modulation may be readily determined by means of one or more lymphocyte activation tests. Usually activation is measured via lymphocyte proliferation. Inhibition of proliferation thus always means immunosuppression under the experimental conditions applied. There exist different stimuli for lymphocyte activation, in particular:
a) co-culture of lymphocytes of different species (mixed lymphocyte reaction, hereinafter referred as MLR) in a so-called mixed lymphocyte culture test: lymphocytes expressing different minor and major antigens of the HLA-DR type (=alloantigens) activate each other non-specifically;
b) a CD3 assay wherein there is an activation of the T-lymphocytes via an exogenously added antibody (OKT3). This antibody reacts against a CD3 molecule located on the lymphocyte membrane which has a co-stimulatory function. Interaction between OKT3 and CD3 results in T-cell activation which proceeds via the Ca²+/calmodulin/calcineurin system and can be inhibited e.g. by cyclosporin A (hereinafter referred as CyA);
c) a CD28 assay wherein specific activation of the T-lymphocyte proceeds via an exogenously added antibody against a CD28 molecule which is also located on the lymphocyte membrane and delivers strong co-stimulatory signals. This activation is Ca²+-independent and thus cannot be inhibited by CyA.

Determination of the immunosuppressing or immunomodulating activity of the pyrido(3,2-d)pyrimidine derivatives of this invention, as well as synergistic combinations comprising them, is preferably based on the determination of one or more, preferably at least three lymphocyte activation in vitro tests, more preferably including at least one of the MLR test, CD3 assay and CD28 assay referred above. Preferably the lymphocyte activation in vitro tests used include at least two assays for two different clusters of differentiation preferably belonging to the same general type of such clusters and more preferably belonging to type I transmembrane proteins. Optionally the determination of the immuno-suppressing or immunomodulating activity may be performed on the basis of other lymphocyte activation in vitro tests, for instance by performing a TNF-α assay or an IL-1 assay or an IL-6 assay or an IL-10 assay or an IL-12 assay or an assay for a cluster of differentiation belonging to a further general type of such clusters and more preferably belonging to type II transmembrane proteins such as, but not limited to, CD69, CD 71 or CD134.

The synergistic effect may be evaluated by the median effect analysis method described herein before. Such tests may for instance, according to standard practice in the art, involve the use of equiment, such as flow cytometer, being able to separate and sort a number of cell subcategories at the end of the analysis, before these purified batches can be analysed further.

Synergistic activity of the pharmaceutical compositions of this invention in the prevention or treatment of transplant rejection may be readily determined by means of one or more leukocyte activation tests performed in a Whole Blood Assay (hereinafter referred as WBA) described for instance by Lin et al. in Transplantation (1997) 63:1734-1738. WBA used herein is a lymphoproliferation assay performed in vitro using lymphocytes present in the whole blood, taken from animals that were previously given the pyrido(3,2-d)pyrimidine derivative of this invention, and optionally the other immunosuppressant drug, in vivo. Hence this assay reflects the in vivo effect of substances as assessed by an in vitro read-out assay. The synergistic effect may be evaluated by the median effect analysis method described herein before. Various organ transplantation models in animals are also available in vivo, which are strongly influenced by different immunogenicities, depending on the donor and recipient species used and depending on the nature of the transplanted organ. The survival time of transplanted organs can thus be used to measure the suppression of the immune response.

The pharmaceutical composition or combined preparation with synergistic activity against immunosuppression or immunomodulation according to this invention may contain the pyrido(3,2-d)pyrimidine derivative represented by one of the structural formulae (I), (II), (III) and (IV) over a broad content range depending on the contemplated use and the expected effect of the preparation. Typically, the pyrido(3,2-d)pyrimidine derivative content in the combined preparation is within the range of from 0.1 to 99.9% by weight, preferably from 1 to 99% by weight, more preferably from about 5 to 95% by weight.

The invention further relates to a composition or combined preparation having synergistic effects against cell proliferation and containing:
(a) one or more antineoplastic drugs, and
(b) at least one pyrido(3,2-d)pyrimidine derivative represented by the structural formula (I), and
(c) optionally one or more pharmaceutical excipients or pharmaceutically acceptable carriers,
   for simultaneous, separate or sequential use in the treatment or prevention of cell proliferative disorders.

Suitable antineoplastic drugs for inclusion into the synergistic antiproliferative pharmaceutical compositions or combined preparations of this invention are preferably selected from the group consisting of alkaloids, alkylating agents (including but not limited to alkyl sulfonates, aziridines, ethylenimines, methylmelamines, nitrogen mustards and nitrosoureas), antibiotics, antimetabolites (including but not limited to folic acid analogues, purine analogs and pyrimidine analogues), enzymes, interferon and platinum complexes. More specific examples include acivicin; aclarubicin; acodazole; acronine; adozelesin; aldesleukin; altretamine; ambomycin; ametantrone; aminoglutethimide; amsacrine; anastrozole; anthramycin; asparaginase; asperlin; azacitidine; azetepa; azotomycin; batimastat; benzodepa; bicalutamide; bisantrene; bisnafide; bizelesin; bleomycin; brequinar; bropirimine; busulfan; cactinomycin; calusterone; caracemide; carbetimer; carboplatin; carmustine; carubicin; carzelesin; cedefingol; chlorambucil; cirolemycin; cisplatin; cladribine; crisnatol; cyclophosphamide; cytarabine; dacarbazine; dactinomycin; daunorubicin; decitabine; dexormaplatin; dezaguanine; diaziquone; docetaxel; doxorubicin; droloxifene; dromostanolone; duazomycin; edatrexate; eflomithine; elsamitrucin; enloplatin; enpromate; epipropidine; epirubicin; erbulozole; esorubicin; estramustine; etanidazole; ethiodized oil I¹³¹; etoposide; etoprine; fadrozole; fazarabine; fenretinide; floxuridine; fludarabine; fluorouracil; fluorocitabine; fosquidone; fostriecin; gemcitabine; Gold 198; hydroxyurea; idarubicin; ifosfamide; ilmofosine; interferon α-2a; interferon α-2b; interferon α-n1; interferon α-n3; interferon β-1a; interferon γ-1b; iproplatin; irinotecan; lanreotide; letrozole; leuprolide; liarozole; lometrexol; lomustine; losoxantrone; masoprocol; maytansine; mechlorethamine; megestrol; melengestrol; melphalan; menogaril; mercaptopurine; methotrexate; metoprine; meturedepa; mitindomide; mitocarcin; mitocromin; mitogillin; mitomalcin; mitomycin; mitosper; mitotane; mitoxantrone; mycophenolic acid; nocodazole; nogala-mycin; ormaplatin; oxisuran; paclitaxel; pegaspargase; peliomycin; pentamustine; peplomycin; perfosfamide; pipobroman; piposulfan; piroxantrone; plicamycin; plomestane; porfimer; porfiromycin; prednimustine; procarbazine; puromycin; pyrazofurin; riboprine; rogletimide; safingol; semustine; simtrazene; sparfosate; sparsomycin; spirogermanium; spiromustine; spiroplatin; streptonigrin; streptozocin; strontium 89 chloride; sulofenur; talisomycin; taxane; taxoid; tecogalan; tegafur; teloxantrone; temoporfin; teniposide; teroxirone; testolactone; thiamiprine; thioguanine; thiotepa; tiazofurin; tirapazamine; topotecan; toremifene; trestolone; triciribine; trimetrexate; triptorelin; tubulozole; uracil mustard; uredepa; vapreotide; verteporfin; vinblastine; vincristine; vindesine; vinepidine; vinglycinate; vinleurosine; vinorelbine; vinrosidine; vinzolidine; vorozole; zeniplatin; zinostatin; zorubicin; and their pharmaceutically acceptable salts.

Other suitable anti-neoplastic compounds include vitamin D3 derivatives such as, but not limited to, 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; anti-androgens such as, but not limited to, benorterone, cioteronel, cyproterone, delmadinone, oxendolone, topterone, zanoterone and their pharmaceutically acceptable salts; anti-estrogens such as, but not limited to, clometherone; delmadinone; nafoxidine; nitromifene; raloxifene; tamoxifen; toremifene; trioxifene and their pharmaceutically acceptable salts; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin; azasetron; azatoxin; azatyrosine; baccatin III derivatives; balanol; batimastat; BCR/ABL antagonists; benzochlorins; benzoylstaurosporine; β-lactam derivatives; β-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors; castanospermine; cecropin B; cetrorelix; chlorins; chloroquinoxaline sulfonamide; cicaprost; cis-porphyrin; clomifene and analogues thereof; clotrimazole; collismycin A and B; combretastatin and analogues thereof; conagenin; crambescidin 816; cryptophycin and derivatives thereof; curacin A; cyclopentanthraquinones; cycloplatam; cypemycin; cytarabine; cytolytic factor; cytostatin; dacliximab; dehydrodidemnin B; deslorelin; dexifosfamide; dexrazoxane; dexverapamil; didemnin B; didox; diethylnorspermine; dihydro-5-azacytidine; dihydrotaxol; dioxamycin; diphenyl spiromustine; docosanol; dolasetron; doxifluridine; droloxifene; dronabinol; duocarmycin SA; ebselen; ecomustine; edelfosine; edrecolomab; elemene; emitefur; epristeride; estrogen agonists and antagonists; exemestane; filgrastim; finasteride; flavopiridol; flezelastine; fluasterone; fluorodaunorunicin; forfenimex; formestane; fotemustine; gadolinium texaphyrin; gallium nitrate; galocitabine; ganirelix; gelatinase inhibitors; glutathione inhibitors; hepsulfam; heregulin; hexamethylene bisacetamide; hypericin; ibandronic acid; idoxifene; idramantone; ilomastat; imidazoacridones; imiquimod; immunostimulant peptides; insulin-like growth factor-1 receptor inhibitor; interferon agonists; iobenguane; iododoxorubicin; ipomeanol; irinotecan; iroplact; irsogladine; isobengazole; isohomohalicondrin B; itasetron; jasplakinolide; kahalalide F; lamellarin-N; leinamycin; lenograstim; lentinan; leptolstatin; leukemia inhibiting factor; leuprorelin; levamisole; liarozole; lissoclinamide; lobaplatin; lombricine; lonidamine; lovastatin; loxoribine; lurtotecan; lutetium texaphyrin; lysofylline; mannostatin A; marimastat; masoprocol; maspin; matrilysin inhibitors; matrix metalloproteinase inhibitors; merbarone; meterelin; methioninase; metoclopramide; MIF inhibitors; mifepristone; miltefosine; mirimostim; mitoguazone; mitolactol; mitonafide; mitotoxin fibroblast growth factor-saporin; mofarotene; molgramostim; human chorionic gonadotrophin monoclonal antibody; mopidamol; mycaperoxide B; myriaporone; N-acetyldinaline; N-substituted benzamides; nafarelin; nagrestip; naloxone; pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; octreotide; okicenone; onapristone; ondansetron; ondansetron; oracin; osaterone; oxaliplatin; oxaunomycin; palauamine; palmitoylrhizoxin; pamidronic acid; panaxytriol; panomifene; parabactin; pazelliptine; peldesine; pentosan; pentostatin; pentrozole; perflubron; perillyl alcohol; phenazinomycin; phenylacetate; phosphatase inhibitors; picibanil; pilocarpine; pirarubicin; piritrexim; placetin A and B; plasminogen activator inhibitor; propyl bis-acridone; prostaglandin J2; proteasome inhibitors; protein kinase C inhibitors; protein tyrosine phosphatase inhibitors; purine nucleoside phosphorylase inhibitors; purpurins; pyrazoloacridine; raltitrexed; ramosetron; ras farnesyl protein transferase inhibitors; ras inhibitors; ras-GAP inhibitors; retelliptine; rhenium 186 etidronate; rhizoxin; retinamide; rohitukine; romurtide; roquinimex; rubiginone B1; ruboxyl; saintopin; sarcophytol A; sargramostim; sizofuran; sobuzoxane; sodium borocaptate; sodium phenylacetate; solverol; somatomedin binding protein; sonermin; sparfosic acid; spicamycin D; splenopentin; spongistatin 1; squalamine; stem-cell division inhibitors; stipiamide; stromelysin inhibitors; sulfinosine; suradista; suramin; swainsonine; tallimustine; tamoxifen; tauromustine; tazarotene; tecogalan; tellurapyrylium; telomerase inhibitors; temozolomide; tetrachlorodecaoxide; tetrazomine; thaliblastine; thiocoraline; thrombopoietin; thymalfasin; thymopoietin receptor agonist; thymotrinan; thyroid stimulating hormone; tin ethyl etiopurpurin; titanocene; topsentin; tretinoin; triacetyluridine; tropisetron; turosteride; tyrosine kinase inhibitors; tyrphostins; ubenimex; urogenital sinus-derived growth inhibitory factor; urokinase receptor antagonists; variolin B; velaresol; veramine; verdins; verteporfin; vinxaltine; vitaxin; zanoterone; zilascorb; and their pharmaceutically acceptable salts.

The compounds of this invention may also be administered in combination with anti-cancer agents which act by arresting cells in the G2-M phases due to stabilized microtubules. In addition to Taxol (paclitaxel), analogues and derivatives thereof, other examples of anti-cancer agents which act by this mechanism include without limitation the following marketed drugs and drugs in development: erbulozole, dolastatin, mivobulin isethionate, discodermolide, altorhyrtins, spongistatins, cemadotin hydrochloride, epothilones desoxyepothilone, 16-aza-epothilone, 21-aminoepothilone, 21-hydroxyepothilone, 26-fluoroepothilone, auristatin, soblidotin, cryptophycin, vitilevuamide, tubulysin, canadensol, centaureidin, oncocidin, fijianolide, laulimalide, narcosine, nascapine, hemiasterlin, vanadocene acetylacetonate, monsatrol, inanocine, eleutherobins, caribaeoside, caribaeolin, halichondrin, diazonamide, taccalonolide, diozostatin, phenylahistin, myoseverin, resverastatin phosphate sodium, and their pharmaceutically acceptable salts.

Synergistic activity of the pharmaceutical compositions or combined preparations of this invention against cell proliferation may be readily determined by means of one or more tests such as, but not limited to, the measurement of the radioactivity resulting from the incorporation of ³H-thymidine in culture of tumor cell lines. For instance, different tumor cell lines may be selected in order to evaluate the anti-tumor effects of the test compounds, such as but not limited to:
RPM11788: human Peripheral Blood Leucocytes (PBL) Caucasian tumor line,
Jurkat: human acute T cell leukemia,
EL4: C57BI/6 mouse lymphoma, or
THP-1: human monocyte tumor line.
   Depending on the selected tumor cell line, and according to general knowledge in the art, various culture media may be used for such tests, such as for example:
for RPMI1788 and THP-1: RPMI-1640+10% FCS+1% NEAA+1% sodium pyruvate+5×10⁵ mercapto-ethanol+antibiotics (G-418 0.45 μg/ml).
for Jurkat and EL4: RPMI-1640+10% FCS+antibiotics (G-418 0.45 μg/ml).

In a specific embodiment of the cell proliferation synergy determination test, tumor cell lines are harvested and a suspension of 0.27×10⁶ cells/ml in whole medium is prepared. The suspensions (150 μl) are added to a microtiter plate in triplicate. Either complete medium (controls) or the test compounds at the test concentrations (50 μl) are added to the cell suspension in the microtiter plate. Cells are incubated at 37° C. under 5% CO₂ for about 16 hours. ³H-thymidine is added, and cells are incubated for another 8 hours and then harvested, and radioactivity is measured in counts per minute (CPM) in a β-counter. The ³H-thymidine cell content, and thus the measured radioactivity, is proportional to the proliferation of the cell lines. The synergistic effect is evaluated by the median effect analysis method as disclosed herein before.

The pharmaceutical composition or combined preparation with synergistic activity against cell proliferation according to this invention may contain the pyrido(3,2-d)pyrimidine derivative represented by the structural formula (I) over a broad content range depending on the contemplated use and the expected effect of the preparation. Typically, the pyrido(3,2-d)pyrimidine derivative content of the combined preparation is within the range of from 0.1 to 99.9% by weight, preferably from 1 to 99% by weight, more preferably from about 5 to 95% by weight.

The invention further relates to a pharmaceutical composition or combined preparation having synergistic effects against a viral infection and containing:
(a) one or more anti-viral agents, and
(b) at least one pyrido(3,2-d)pyrimidine derivative represented by the structural formula (I), and
(c) optionally one or more pharmaceutical excipients or pharmaceutically acceptable carriers,
   for simultaneous, separate or sequential use in the treatment or prevention of a viral infection.

Suitable anti-viral agents for inclusion into the synergistic antiviral compositions or combined preparations of this invention include, for instance, retroviral enzyme inhibitors belonging to categories well known in the art, such as HIV-1 IN inhibitors, nucleoside reverse transcriptase inhibitors (e.g. zidovudine, lamivudine, didanosine, stavudine, zalcitabine and the like), non-nucleoside reverse transcriptase inhibitors (e.g. nevirapine, delavirdine and the like), other reverse transcriptase inhibitors (e.g. foscamet sodium and the like), and HIV-1 protease inhibitors (e.g. saquinavir, ritonavir, indinavir, nelfinavir and the like). Other suitable antiviral agents include for instance acemannan, acyclovir, adefovir, alovudine, alvircept, amantadine, aranotin, arildone, atevirdine, pyridine, cidofovir, cipamfylline, cytarabine, desciclovir, disoxaril, edoxudine, enviradene, enviroxime, famciclovir, famotine, fiacitabine, fialuridine, floxuridine, fosarilate, fosfonet, ganciclovir, idoxuridine, kethoxal, lobucavir, memotine, methisazone, penciclovir, pirodavir, somantadine, sorivudine, tilorone, trifluridine, valaciclovir, vidarabine, viroxime, zinviroxime, moroxydine, podophyllotoxin, ribavirine, rimantadine, stallimycine, statolon, tromantadine and xenazoic acid, and their pharmaceutically acceptable salts.

Especially relevant to this aspect of the invention is the inhibition of the replication of viruses selected from the group consisting of picorna-, toga-, bunya, orthomyxo-, paramyxo-, rhabdo-, retro-, arena-, hepatitis B-, hepatitis C-, hepatitis D-, adeno-, vaccinia-, papilloma-, herpes-, corona-, varicelia- and zoster-virus, in particular human immunodeficiency virus (HIV). Synergistic activity of the pharmaceutical compositions or combined preparations of this invention against viral infection may be readily determined by means of one or more tests such as, but not limited to, the isobologram method, as previously described by Elion et al. in J. Biol. Chem. (1954) 208:477-488 and by Baba et al. in Antimicrob. Agents Chemother. (1984) 25:515-517, using EC₅₀ for calculating the fractional inhibitory concentration (hereinafter referred as FIC). When the minimum FIC index corresponding to the FIC of combined compounds (e.g., FICₓ+FIC_{y}) is equal to 1.0, the combination is said to be additive; when it is between 1.0 and 0.5, the combination is defined as sub-synergistic, and when it is lower than 0.5, the combination is by defined as synergistic. When the minimum FIC index is between 1.0 and 2.0, the combination is defined as subantagonistic and, when it is higher than 2.0, the combination is defined as antagonistic.

The pharmaceutical composition or combined preparation with synergistic activity against viral infection according to this invention may contain the pyrido(3,2-d)pyrimidine derivative represented by the structural formula (I) over a broad content range depending on the contemplated use and the expected effect of the preparation. Typically, the pyrido(3,2-d)pyrimidine derivative content of the combined preparation is within the range of from 0.1 to 99.9% by weight, preferably from 1 to 99% by weight, more preferably from about 5 to 95% by weight.

The invention further relates to a pharmaceutical composition or combined preparation having synergistic effects against a disease mediated by phosphodiesterase-4 activity and containing:
(a) one or more phosphodiesterase-4 inhibitors, and
(b) at least one pyrido(3,2-d)pyrimidine derivative represented by the structural formula (I), and
(c) optionally one or more pharmaceutical excipients or pharmaceutically acceptable carriers,
   for simultaneous, separate or sequential use in the treatment or prevention of a disease mediated by phosphodiesterase-4 activity. The pharmaceutical composition or combined preparation with synergistic activity against a disease mediated by phosphodiesterase-4 activity according to this invention may contain the pyrido(3,2-d)pyrimidine derivative represented by the structural formula (I) over a broad content range depending on the contemplated use and the expected effect of the preparation. ally, the pyrido(3,2-d)pyrimidine derivative content of the combined preparation is within the range of from 0.1 to 99.9% by weight, preferably from 1 to 99% by weight, more preferably from about 5 to 95% by weight.

Suitable phosphodiesterase inhibitors may be selected from the group consisting of pyrrolidinones (such as, but not limited to, rolipram, RO20-1724 and RS 33793), quinazolinediones (such as, but not limited to, nitraquazone, CP-77059 and RS-25344), xanthine derivatives (such as, but not limited to, denbufylline, arofylline and BRL 61063), phenylethyl pyridines (such as, but not limited to, CDP 840), tetrahydropyrimidones (such as, but not limited to, atizoram), diazepine derivatives (such as, but not limited to, CI 1018), oxime carbamates (such as, but not limited to, filaminast), naphthyridinones (such as, but not limited to, RS 17597), benzofurans (such as, but not limited to, 2-butyl-7-methoxy-benzofuran-4-carboxylic acid (3-5-dichloropyridin-4-yl)-amide, 2-benzyl-7-methoxy-benzofuran-4-carboxylic acid (3-5-dichloropyridin-4-yl)-amide, 7-methoxy-2-phenethyl-benzofuran-4-carboxylic acid (3-5-dichloropyridin-4-yl)-amide, 5-(2-butyl-7-methoxy-benzofuran-4-yl)-tetrahydropyrimidin-2-one, and phenyldihydrobenzofuranes), naphthalene derivatives (such as, but not limited to, T 440), purine derivatives (such as, but not limited to, V-112294A), imidazolidinones, cyclohexane carboxylic acids (such as, but not limited to, ariflo), benzamides (such as, but not limited to, piclamilast), pyridopyridazinones, benzothiophenes (such as, but not limited to, tibenelast), etazolate, S-(+)-glaucine, substituted phenyl compounds and substituted biphenyl compounds, and pyridopyridazinones.

The pharmaceutical compositions and combined preparations according to this invention may be administered orally or in any other suitable fashion. Oral administration is preferred and the preparation may have the form of a tablet, aqueous dispersion, dispersable powder or granule, emulsion, hard or soft capsule, syrup, elixir or gel. The dosing forms may be prepared using any method known in the art for manufacturing these pharmaceutical compositions and may comprise as additives sweeteners, flavoring agents, coloring agents, preservatives and the like. Carrier materials and excipients are detailed hereinbelow and may include, inter alia, calcium carbonate, sodium carbonate, lactose, calcium phosphate or sodium phosphate; granulating and disintegrating agents, binding agents and the like. The pharmaceutical composition or combined preparation of this invention may be included in a gelatin capsule mixed with any inert solid diluent or carrier material, or has the form of a soft gelatin capsule, in which the ingredient is mixed with a water or oil medium. Aqueous dispersions may comprise the biologically active composition or combined preparation in combination with a suspending agent, dispersing agent or wetting agent. Oil dispersions may comprise suspending agents such as a vegetable oil. Rectal administration is also applicable, for instance in the form of suppositories or gels. Injection (e.g. intramuscularly or intraperiteneously) is also applicable as a mode of administration, for instance in the form of injectable solutions or dispersions, depending upon the disorder to be treated and the condition of the patient.

Auto-immune disorders to be prevented or treated by the pharmaceutical compositions or combined preparations of this invention include both:
systemic auto-immune diseases such as, but not limited to, lupus erythematosus, psoriasis, vasculitis, polymyositis, scleroderma, multiple sclerosis, ankylosing spondilytis, rheumatoid arthritis and Sjögren syndrome; auto-immune endocrine disorders such as thyroiditis; and
organ-specific auto-immune diseases such as, but not limited to, Addison disease, hemolytic or pernicious anemia, Goodpasture syndrome, Graves disease, idiopathic thrombocytopenic purpura, insulin-dependent diabetes mellitus, juvenile diabetes, uveitis, Crohn's disease, ulcerative colitis, pemphigus, atopic dermatitis, autoimmune hepatitis, primary biliary cirrhosis, autoimmune pneumonitis, autoimmune carditis, myasthenia gravis, glomerulonephritis and spontaneous infertility.

Transplant rejections to be prevented or treated by the pharmaceutical compositions or combined preparations of this invention include the rejection of transplanted or grafted organs or cells (both allografts and xenografts), such as but not limited to host versus graft reaction disease. The term “organ” as used herein means all organs or parts of organs in mammals, in particular humans, such as but not limited to kidney, lung, bone marrow, hair, cornea, eye (vitreous), heart, heart valve, liver, pancreas, blood vessel, skin, muscle, bone, intestine or stomach. The term “rejection” as used herein means all reactions of the recipient body or the transplanted organ which in the end lead to cell or tissue death in the transplanted organ or adversely affect the functional ability and viability of the transplanted organ or the recipient. In particular, this means acute and chronic rejection reactions. Also included in this invention is preventing or treating the rejection of cell transplants and xenotransplantation. The major hurdle for xenotransplantation is that even before the T lymphocytes, responsible for the rejection of allografts, are activated, the innate immune system, especially T-independent B lymphocytes and macrophages are activated. This provokes two types of severe and early acute rejection called hyper-acute rejection and vascular rejection, respectively. The present invention addresses the problem that conventional immunosuppressant drugs like cyclosporin A are ineffective in xeno-transplantation. The ability of the compounds of this invention to suppress T-independent xeno-antibody production as well as macrophage activation may be evaluated in the ability to prevent xenograft rejection in athymic, T-deficient mice receiving xenogenic hamster-heart grafts.

In a particular embodiment of the invention, the pyrido(3,2-d)pyrimidine derivatives according to one of the structural formulae (II), (III) and (IV) may be used in the treatment of auto-immune disorders, or the prevention of a transplant rejection in a patient. In particular, pyrido(3,2-d)pyrimidine derivatives according to one of the structural formulae (II), (III) and (IV) may be used in the treatment a disease selected from the group consisting of rheumatoid arthritis, Crohn's disease, ulcerative colitis, uveitis, multiple sclerosis, atopic dermatis, psoriasis and lupus erythematosus. Cell proliferative disorders to be prevented or treated by the pharmaceutical compositions or combined preparations including a pyrido(3,2-d)pyrimidine derivative represented by the structural formula (I) of this invention include any kind of tumor progression or invasion or metastasis inhibition of a cancer, preferably one selected from the group consisting of lung cancer, leukaemia, ovarian cancer, sarcoma, Kaposi's sarcoma, meningioma, colon cancer, lymp node tumor, glioblastoma multiforme, prostate cancer or skin carcinose.

CNS disorders to be prevented or treated by the pharmaceutical compositions or combined preparations including a pyrido(3,2-d)pyrimidine derivative represented by the structural formula (I) of this invention include cognitive pathologies such as dementia, cerebral ischemia, trauma, epilepsy, schizophrenia, chronic pain, and neurologic disorders such as but not limited to depression, social phobia and obsessive compulsive disorders.

Cardiovascular disorders to be prevented or treated by the pharmaceutical compositions or combined preparations including a pyrido(3,2-d)pyrimidine derivative represented by the structural formula (I) of this invention include, but are not limited to, ischemic disorders, infarct or reperfusion damage, atherosclerosis and stroke. TNF-α-related disorders to be prevented or treated by the pharmaceutical compositions or combined preparations including a pyrido(3,2-d)pyrimidine derivative represented by the structural formula (I) of this invention include the following:
septic or endotoxic shock or sepsis, especially in patients with a serum level of interleukin-6 above 1,000 μg/ml at start of treatment;
vascular TNF-α-mediated diseases such as, but not limited to, disseminated intravascular coagulation and Kawasaki's pathology;
pathologies and conditions associated with and/or induced by abnormal levels of TNF-α (herein defined as exceeding by at least 10% and at most 500% the TNF-α level present in a normal healthy subject) occurring in a systemic, localized or particular tissue type or location in the body of the mammal; such tissue types include, but are not limited to, blood, lymph, liver, kidney, spleen, heart muscle or blood vessels, brain or spinal cord white matter or grey matter, cartilage, ligaments, tendons, lung, pancreas, ovary, testes and prostate. Abnormal TNF-α levels can also be localized to specific regions or cells in the body, such as joints, nerve blood vessel junctions and bones. Such pathologies include alcohol-induced hepatitis; neurodegenerative diseases such as extrapyramidal and cerebellar disorders including lesions of the corticospinal system; disorders of the basal ganglia; hyperkinetic movement disorders such as chorea; drug-induced movement disorders; hypokinetic movement disorders, such as Parkinson's disease; spinocerebellar degenerations such as spinal ataxia, multiple systems degenerations (including Dejerine-Klumpke syndrome) and systemic disorders (including Refsum's disease, abetalipoprotemia, ataxia and telangiectasia); disorders of the motor unit, such as neurogenic muscular atrophies (anterior horn cell degeneration, such as amyotrophic lateral sclerosis, infantile spinal muscular atrophy and juvenile spinal muscular atrophy); Alzheimer's disease;
Wernicke-Korsakoff syndrome; Creutzfeldt-Jakob disease; Hallerrorden-Spatz disease; and primary or secondary myelodysplastic syndromes;
toxic effects of TNF-α and/or anti-cancer chemotherapeutic agents, especially side effects associated with TNF-α generation during neoplastic therapy, for instance following use of cisplatin;
injuries after irradiation of a tissue of a mammal by radio-elements, such as but not limited to radiation-induced graft-versus-host disease; and
cachexia and similar chronic wasting diseases, whether associated with cancer or with other chronic diseases such as malabsortive disorders, excessive physical stress, eating disorders, and AIDS.

Disorders mediated by phosphodiesterase-4 activity to be prevented or treated by the pharmaceutical compositions or combined preparations including a pyrido(3,2-d)pyrimidine derivative represented by the structural formula (I) of this invention include, but are not limited to, erectile dysfunction, sepsis and septic shock. PDE-4 is particularly abundant in inflammatory and immune cells. Through modulation of cAMP levels, PDE-4 regulates leukocyte responses including the pro-inflammatory actions of monocytes, T cells and neutrophils, airway and vascular smooth muscle constriction, and neurotransmitter signaling through adenylyl cyclase linked G-protein coupled receptors (such as that for N-methyl-D-aspartate). Inhibition of PDE-4 blocks cell traffic and cell proliferation, and attenuates the production of inflammatory mediators, cytokines and reactive oxygen species. TNF-α is an important target in rheumatoid arthritis, ankylosing spondylitis, Crohn's disease and psoriasis. However, in diseases such as severe asthma and late-stage rheumatoid arthritis, neutrophils do play a key role in the pathological inflammatory process. PDE-4 inhibitors are able to suppress multiple neutrophil responses, including the production of IL-8, leukotriene B4 and superoxide anions, as well as degranulation, chemotaxis and adhesion. In addition, the smooth muscle (e.g. bronchodilatory) relaxing effect of PDE-4 inhibitors are very beneficial for the treatment of asthma. The inhibition of TNF-α production that follows inhibition of PDE-4 β isoform is cAMP-dependent and requires protein kinase A activity for protection from LPS-induced shock. The highly specialized function of PDE-4 B in macrophages and its critical role in LPS signaling are thus well known in the art, and therefore provide basis for a therapeutic strategy using subtype-selective PDE-4 inhibitors for the treatment of sepsis and septic shock.

The term “erectile dysfunction” as used herein includes any type of erectile dysfunction, such as but not limited to vasculogenic, neurogenic, endocrinologic and psychogenic impotence (“impotence” being used herein to indicate a periodic or consistent inability to achieve or sustain an erection of sufficient rigidity for sexual intercourse); Peyronie's syndrome; priapism; premature ejaculation; and any other condition, disease or disorder, regardless of cause or origin, which interferes with at least one of the three phases of human sexual response, i.e., desire, excitement and orgasm.

The medicament of this invention may be for prophylactic use, i.e. where circumstances are such that an elevation in the TNF-α level might be expected or alternatively, may be for use in reducing the TNF-α level after it has reached an undesirably high level (as defined herein above) or as the TNF-α level is rising.

The term “pharmaceutically acceptable carrier or excipient” as used herein in relation to pharmaceutical compositions and combined preparations means any material or substance with which the active principle, i.e. a pyrido(3,2-d)pyrimidine derivative represented by one of the structural formulae (I), (II), (III) and (IV), and optionally the immunosuppressant or immunomodulator or antineoplastic drug or antiviral agent, may be formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, pellets or powders.

Suitable pharmaceutical carriers for use in the said pharmaceutical compositions and their formulation are well known to those skilled in the art. There is no particular restriction to their selection within the present invention although, due to the usually low or very low water-solubility of the pyrido(3,2-d)pyrimidine derivatives of this invention, special attention will be paid to the selection of suitable carrier combinations that can assist in properly formulating them in view of the expected time release profile. Suitable pharmaceutical carriers include additives such as wetting agents, dispersing agents, stickers, adhesives, emulsifying or surface-active agents, thickening agents, complexing agents, gelling agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals.

The pharmaceutical compositions of the present invention may be prepared in any known manner, for instance by homogeneously mixing, dissolving, spray-drying, coating and/or grinding the active ingredients, in a one-step or a multi-steps procedure, with the selected carrier material and, where appropriate, the other additives such as surface-active agents. may also be prepared by micronisation, for instance in view to obtain them in the form of microspheres usually having a diameter of about 1 to 10 μm, namely for the manufacture of microcapsules for controlled or sustained release of the biologically active ingredient(s).

Suitable surface-active agents to be used in the pharmaceutical compositions of the present invention are non-ionic, cationic and/or anionic surfactants having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanyl-phosphatidylcholine, dipalmitoylphoshatidylcholine and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediamino-polypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, preferably halides, having four hydrocarbon radicals optionally substituted with halo, phenyl, substituted phenyl or hydroxy; for instance quaternary ammonium salts containing as N-substituent at least one C₈-C₂₂ alkyl radical (e.g. cetyl, lauryl, palmityl, myristyl, oleyl and the like) and, as further substituents, unsubstituted or halogenated lower alkyl, benzyl and/or hydroxy-C₁₋₄ alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch”, 2^{nd} ed. (Hanser Verlag, Vienna, 1981) and “Encyclopaedia of Surfactants (Chemical Publishing Co., New York, 1981).

Structure-forming, thickening or gel-forming agents may be included into the pharmaceutical compositions and combined preparations of the invention. Suitable such agents are in particular highly dispersed silicic acid, such as the product commercially available under the trade name Aerosil; bentonites; tetraalkyl ammonium salts of montmorillonites (e.g., products commercially available under the trade name Bentone), wherein each of the alkyl groups may contain from 1 to 20 carbon atoms; cetostearyl alcohol and modified castor oil products (e.g. the product commercially available under the trade name Antisettle).

Gelling agents which may be included into the pharmaceutical compositions and combined preparations of the present invention include, but are not limited to, cellulose derivatives such as carboxymethylcellulose, cellulose acetate and the like; natural gums such as arabic gum, xanthum gum, tragacanth gum, guar gum and the like; gelatin; silicon dioxide; synthetic polymers such as carbomers, and mixtures thereof. Gelatin and modified celluloses represent a preferred class of gelling agents.

Other optional excipients which may be included in the pharmaceutical compositions and combined preparations of the present invention include additives such as magnesium oxide; azo dyes; organic and inorganic pigments such as titanium dioxide; UV-absorbers; stabilisers; odor masking agents; viscosity enhancers; antioxidants such as, for example, ascorbyl palmitate, sodium bisulfite, sodium metabisulfite and the like, and mixtures thereof; preservatives such as, for example, potassium sorbate, sodium benzoate, sorbic acid, propyl gallate, benzylalcohol, methyl paraben, propyl paraben and the like; sequestering agents such as ethylene-diamine tetraacetic acid; flavoring agents such as natural vanillin; buffers such as citric acid and acetic acid; extenders or bulking agents such as silicates, diatomaceous earth, magnesium oxide or aluminum oxide; densification agents such as magnesium salts; and mixtures thereof.

Additional ingredients may be included in order to control the duration of action of the biologically-active ingredient in the compositions and combined preparations of the invention. Control release compositions may thus be achieved by selecting appropriate polymer carriers such as for example polyesters, polyamino-acids, polyvinyl-pyrrolidone, ethylene-vinyl acetate copolymers, methylcellulose, carboxy-methylcellulose, protamine sulfate and the like. The rate of drug release and duration of action may also be controlled by incorporating the active ingredient into particles, e.g. microcapsules, of a polymeric substance such as hydrogels, polylactic acid, hydroxymethyl-cellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition or combined preparation of the invention may also require protective coatings.

Pharmaceutical forms suitable for injectable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol, complexing agents such as cyclodextrins and the like, and mixtures thereof.

Pharmaceutical forms suitable for transurethral delivery, e.g. intracavernosal injection, such as needed for the treatment of erectile dysfunction are extensively disclosed in U.S. Pat. No. 6,127,363, the content of which is incorporated by reference. Transurethral drug delivery may involve an active delivery mechanism such as iontophoresis, electroporation or phonophoresis. Devices and methods for delivering drugs in this way are well known in the art. Iontophoretically assisted drug delivery is, for example, described in WO96/40054. Briefly, the active agent is driven through the urethral wall by means of an electric current passed from an external electrode to a second electrode contained within or affixed to a urethral probe.

Other modes of local drug administration can also be used. For example, the selected active agent may be administered by way of intracavernosal injection, or may be administered topically, in an ointment, gel or the like, or transdermally, including transscrotally, using a conventional transdermal drug delivery system. Intracavernosal injection can be carried out by use of a syringe or any other suitable device. An example of a hypodermic syringe useful herein is described in U.S. Pat. No. 4,127,118, injection being made on the dorsum of the penis by placement of the needle to the side of each dorsal vein and inserting it deep into the corpora.

For intracavernosal injection, the active agent to be administered is preferably incorporated into a sterile liquid preparation, typically a solution or suspension in an aqueous or oleaginous medium. This solution or suspension may be formulated according to techniques known in the art using suitable carriers, dispersants, wetting agents, diluents, suspending agents or the like. Among the acceptable vehicles and solvents that may be employed are water, isotonic saline, vegetable oil, fatty esters and polyols.

Since, in the case of combined preparations including the pyrido(3,2-d)pyrimidine derivative of this invention and an immunosuppressant or immunomodulator or antineoplastic drug or antiviral agent or phosphodiesterase-4 inhibitor, both ingredients do not necessarily bring out their synergistic therapeutic effect directly at the same time in the patient to be treated, the said combined preparation may be in the form of a medical kit or package containing the two ingredients in separate but adjacent form. In the latter context, each ingredient may therefore be formulated in a way suitable for an administration route different from that of the other ingredient, e.g. one of them may be in the form of an oral or parenteral formulation whereas the other is in the form of an ampoule for intravenous injection or an aerosol.

The present invention further relates to a method for preventing or treating a disease selected from the group consisting of CNS disorders, cell proliferative disorders, viral infections, immune and auto-immune disorders, transplant rejections, PDE-4-mediated diseases and TNF-α-related disorders in a patient, preferably a mammal, more preferably a human being. The method of this invention consists of administering to the patient in need thereof an effective amount of a pyrido(3,2-d)pyrimidine derivative represented by the structural formula (I), (II), (III) or (IV), optionally together with an effective amount of another immunosuppressant or immunomodulator or antineoplastic drug or antiviral agent or phosphodiesterase-4 inhibitor, or a pharmaceutical composition comprising the same, such as disclosed above in extensive details. The effective amount is usually in the range of about 0.01 mg to 20 mg, preferably about 0.1 mg to 5 mg, per day per kg bodyweight for humans. Depending upon the pathologic condition to be treated and the patient's condition, the said effective amount may be divided into several sub-units per day or may be administered at more than one day intervals. The patient to be treated may be any warm-blooded animal, preferably a mammal, more preferably a human being, suffering from said pathologic condition.

The preferred compounds of the present invention are non-sedating. In other words, a dose of such compounds that is twice the minimum dose sufficient to provide analgesia in an animal model for determining pain relief causes only transient (i.e. lasting for no more than half the time that pain relief lasts) or preferably no statistically significant sedation in an animal model assay of sedation (using the method described by Fitzgerald et al. in Toxicology (1988) 49:433-9). Preferably, a dose that is five times the minimum dose sufficient to provide analgesia does not produce statistically significant sedation. More preferably, a compound provided herein does not produce sedation at intravenous doses of less than 10 mg/kg per day or at oral doses of less than 30 mg/kg per day. If desired, compounds provided herein may be evaluated for toxicity (a preferred compound is non-toxic when an immunomodulating amount or a cell anti-proliferative amount is administered to a subject) and/or side effects (a preferred compound produces side effects comparable to placebo when a therapeutically effective amount of the compound is administered to a subject). Toxicity and side effects may be assessed using any standard method. In general, the term “non-toxic” as used herein shall be understood as referring to any substance that, in keeping with established criteria, is susceptible to approval by the United States Federal Drug Administration for administration to mammals, preferably humans. Toxicity may be also evaluated using assays including bacterial reverse mutation assays, such as an Ames test, as well as standard teratogenicity and tumorogenicity assays. Preferably, administration of compounds provided herein within the therapeutic dose ranges disclosed hereinabove does not result in prolongation of heart QT intervals (e.g. as determined by electrocardiography in guinea pigs, minipigs or dogs). When administered daily, such doses also do not cause liver enlargement resulting in an increase of liver to body weight ratio of more than 50% over matched controls in laboratory rodents (e.g. mice or rats). Such doses also preferably do not cause liver enlargement resulting in an increase of liver to body weight ratio of more than 10% over matched untreated controls in dogs or other non-rodent mammals. The preferred compounds of the present invention also do not promote substantial release of liver enzymes from hepatocytes in vivo, i.e. the therapeutic doses do not elevate serum levels of such enzymes by more than 50% over matched untreated controls in vivo in laboratory rodents.

Another embodiment of this invention includes the various precursor or “pro-drug” forms of the compounds of the present invention. It may be desirable to formulate the compounds of the present invention in the form of a chemical species which itself is not significantly biologically-active, but which when delivered to the body of a human being or higher mammal will undergo a chemical reaction catalyzed by the normal function of the body, inter alia, enzymes present in the stomach or in blood serum, said chemical reaction having the effect of releasing a compound as defined herein. The term “pro-drug” thus relates to these species which are converted in vivo into the active pharmaceutical ingredient.

The pro-drugs of the present invention can have any form suitable to the formulator, for example, esters are non-limiting common pro-drug forms. In the present case, however, the pro-drug may necessarily exist in a form wherein a covalent bond is cleaved by the action of an enzyme present at the target locus. For example, a C—C covalent bond may be selectively cleaved by one or more enzymes at said target locus and, therefore, a pro-drug in a form other than an easily hydrolysable precursor, inter alia an ester, an amide, and the like, may be used.

For the purposes of the present invention the term “therapeutically suitable pro-drug” is defined herein as “a compound modified in such a way as to be transformed in vivo to the therapeutically active form, whether by way of a single or by multiple biological transformations, when in contact with the tissues of humans or mammals to which the pro-drug has been administered, and without undue toxicity, irritation, or allergic response, and achieving the intended therapeutic outcome

The present invention will be further described with reference to certain more specific embodiments and examples, but the present invention is not limited thereto but only by the attached claims. The following examples are given by way of illustration only.

### EXAMPLE 1

### Synthesis of 6-chloro-2-carboxamido-3-amino-pyridine

To a solution of 6-chloro-2-cyano-3-nitro-pyridine (3.03 g, 16.5 mmol) in ethanol (166 ml) and H₂O (16 ml) was added iron (165 mmol, 9.2 g) and calcium chloride (2.75 g, 24.8 mmol). The reaction mixture was refluxed for 4 hours and then cooled down to room temperature. The precipitate was filtered off over Celite and the filtrate was evaporated to dryness. The residue was redissolved in ethyl acetate and extracted with brine. The aqueous layer was extracted back with ethyl acetate. The combined organic layers were evaporated in vacuo. The residue was adsorbed on silica and purified by silica gel column chromatography, the mobile phase being a ethyl acetate/hexane mixture in a ratio of 3:7, resulting in the pure title compound (1.89 g, yield 67%) which was characterised by its mass spectrum as follows: MS (m/z): 172, 174 ([M+H]⁺, 100).

### EXAMPLE 2

### Synthesis of 6-chloro-pyrido[3,2-d]pyrimidin-4(3H)-one

A suspension of 6-chloro-2-carboxamido-3-amino-pyridine (1.34 mmol, 230 mg) in triethyl orthoformate (10 ml) was refluxed for 3 hours. A white suspension was formed which was cooled down to room temperature. The precipitate was filtered off and dried under vacuum resulting in the pure title compound (174 mg, yield 72%) which was characterised by its mass spectrum as follows: MS (m/z): 182, 184 ([M+H]⁺, 100).

### EXAMPLE 3

### Preparation of 6-(3,4-dimethoxyphenyl)-pyrido[3,2-d]pyrimidin-4(3H)-one

To a solution of 6-chloro-pyrido[3,2-d]pyrimidin-4(3H)-one (200 mg, 1.1 mmol) in 1,4-dioxane (20 ml) and water (10 ml) was added 3,4-dimethoxyphenyl boronic acid (240 mg, 1.32 mmol), potassium carbonate (380 mg, 2.75 mmol) and tetrakis(triphenylphosphine)palladium(0) (63 mg, 0.055 mmol). The reaction mixture was refluxed for 3 hours, cooled down to room temperature and the solvents were evaporated in vacuo. The residue was adsorbed on silica, purified by silica gel column chromatography (the mobile phase being a acetone/dichloromethane mixture, in a ratio gradually ranging from 30:70 to 40:60) and characterised by its mass spectrum as follows: MS (m/z): 284 ([M+H]⁺, 100).

### EXAMPLE 4

### Preparation of 4-chloro-6-(3,4-dimethoxyphenyl)-pyrido[3,2-d]pyrimidine

To a suspension of 6-(3,4-dimethoxyphenyl)-pyrido[3,2-d]pyrimidin-4(3H)-one (150 mg, 0.53 mmol) in toluene (30 ml) was added phosphorus oxychloride (148 μl, 1.59 mmol) and 2,6-lutidine (185 μl, 1.59 mmol). The reaction mixture was refluxed overnight until a black solution was obtained. After evaporation to dryness, the residue was redissolved in ethyl acetate and extracted with a saturated sodium bicarbonate solution. The combined organic layers were evaporated in vacuo. The residue was purified by silica gel column chromatography, the mobile phase being an ethyl acetate/hexane mixture, in a ratio gradually ranging from 2:8 to 3:7, resulting in the pure title compound (123 mg, yield 77%) which was characterised by its mass spectrum as follows: MS (m/z): 302, 304 ([M+H]⁺, 100).

### EXAMPLE 5

### Synthesis of 4-[(2-phenoxyethyl)-piperazin-1-yl]-6-(3,4-dimethoxyphenyl)-Pyrido[3,2-d]pyrimidine

To a suspension of 4-chloro-6-(3,4-dimethoxyphenyl)-pyrido[3,2-d]pyrimidine (120 mg, 0.398 mmol) in isopropanol (15 ml) was added 1-(2-phenoxyethyl)-piperazine (0.795 mmol, 164 mg). The suspension was stirred at 80° C., after which the suspension became a clear colorless solution. The solvents were evaporated in vacuo. The residue was redissolved in ethyl acetate and extracted with a NaOH solution (1 N). The combined organic layers were evaporated in vacuo and purified by silica gel column chromatography (the mobile phase being a mixture of methanol and dichloromethane in a ratio gradually ranging from 1:99 to 2:98), resulting in the title compound (157 mg, yield 84%) which was characterised by its mass spectrum as follows: MS (m/z): 472 ([M+H]⁺, 100).

### EXAMPLE 6

### Synthesis of 2-carboxamido-3-amino-6-(3,4-dimethoxyphenyl)-pyridine

To a solution of 6-(3,4-dimethoxyphenyl)-3-nitropyridine-2-carbonitrile (1.42 g, 5 mmol) in ethanol (50 ml) and water (5 ml) was added iron (1.39 g, 25 mmol) and calcium chloride (6 mmol, 666 mg). The reaction mixture was refluxed for 1 hour. An additional amount of iron (1.39 g, 25 mmol) was added and the reaction was refluxed for another 3 hours. The reaction was cooled down and filtered over a paper filter, followed by washings with boiling ethyl acetate. The filtrate was evaporated in vacuo and the residue was partitioned between ethyl acetate and water. The organic layers were evaporated to dryness and the residue was purified by silica gel column chromatography (the mobile phase being a mixture of ethyl acetate and hexane in a ratio of 1:1), resulting in the pure title compound (770 mg, yield 56%) which was characterised by its mass spectrum as follows: MS (m/z): 273 [(M+H)⁺, 100).

### EXAMPLE 7

### Preparation of 6-(3,4-dimethoxyphenyl)-pyrido[3,2-d]pyrimidin-4(3H)-one

A suspension of 2-carboxamido-3-amino-6-(3,4-dimethoxyphenyl)-pyridine (770 mg, 2.8 mmol) in triethyl orthoformate (28 ml) was refluxed for 12 hours. Then, the reaction mixture was cooled down and evaporated to dryness. The residue was purified by silica gel column chromatography (the mobile phase being an ethyl acetate/hexane mixture in a ratio gradually ranging from 2:8 to 3:7), resulting in the pure title compound (530 mg, yield 67%) which was characterised by its mass spectrum as follows: MS (m/z): 284 ([M+H)⁺, 100].

### EXAMPLE 8

### Synthesis of 4-(4-[3-methylphenyl)amino]carbonyl]piperazin-1-yl)-6-(3,4-dimethoxyphenyl)-pyrido[3,2-d]pyrimidine

To a suspension of 4-chloro-6-(3,4-dimethoxyphenyl)-pyrido[3,2-d]pyrimidine (227 mg, 0.8 mmol) in isopropanol (20 ml) was added piperazine-1-carboxylic acid m-tolylamide (351 mg, 1.6 mmol). The reaction mixture was stirred for 3 hours at 80° C. Then, the reaction was cooled down and evaporated to dryness. The residue was redissolved in ethyl acetate and extracted with a saturated sodium bicarbonate solution. The combined organic layers were evaporated in vacuo. The crude residue was purified by silica gel column chromatography (the mobile phase being a mixture of methanol and dichloromethane in a ratio gradually ranging from 1:99 to 2:98), resulting in the pure title compound (217 mg, yield 56%) which was characterised by its mass spectrum as follows: MZ (m/z): 485 ([M+H)⁺, 100).

### EXAMPLE 9

### Preparation of 2-methyl-6-(3,4-dimethoxyphenyl)-pyrido[3,2-d]pyrimidin-4(3H)-one

A suspension of 2-carboxamido-3-amino-6-(3,4-dimethoxyphenyl)-pyridine (546 mg, 2 mmol) in triethyl orthoacetate (25 ml) was refluxed for 12 hours. Then, the reaction mixture was cooled down and evaporated to dryness. The residue was purified by silica gel column chromatography (the mobile phase being an ethyl acetate/hexane mixture in a ratio gradually ranging from 2:8 to 3:7), resulting in the pure title compound (437 mg, yield 73%) which was characterised by its mass spectrum as follows: MS (m/z): 297 ([M+H]⁺, 100).

### EXAMPLE 10

### Preparation of 2-methyl-4-chloro-6-(3,4-dimethoxyphenyl)-pyrido[3,2-d]pyrimidine

To a solution of 2-methyl-6-(3,4-dimethoxyphenyl)-pyrido[3,2-d]pyrimidin-4(3H)-one (416 mg, 1.4 mmol) in toluene (28 ml) was added 2,6-lutidine (490 μl, 4.2 mmol) and POCl₃ (4.2 mmol, 385 μl). The mixture was refluxed under nitrogen atmosphere for 5 hours. The reaction mixture was cooled down, diluted with ethyl acetate (50 ml) and extracted with a saturated sodium bicarbonate solution. The combined organic layers were evaporated in vacuo and the residue was purified by silica gel column chromatography (the mobile phase being an ethyl acetate/hexane mixture in a ratio of 15:85), resulting in the pure title compound (330 mg, yield 75%) which was characterised by its mass spectrum as follows: MS (m/z): 316, 318 ([M+H]⁺, 100).

### EXAMPLE 11

### Synthesis of 2-methyl-4-(4-[3-methylphenyl)amino]carbonyl]piperazin-1-yl)-6-(3,4-dimethoxyphenyl)-pyrido[3,2-d]pyrimidine

To a suspension of 2-methyl-4-chloro-6-(3,4-dimethoxyphenyl)-pyrido[3,2-d]pyrimidine (330 mg, 1.04 mmol) in acetonitrile (20 ml) was added piperazine-1-carboxylic acid m-tolylamide (479 mg, 2.2 mmol). The reaction mixture was refluxed for 2 hours. The mixture was cooled down and ethyl acetate was added (100 ml). The reaction mixture was extracted with a saturated sodium bicarbonate solution. The combined organic layers were evaporated to dryness. The residue was purified by a first silica gel column chromatography (the mobile phase being a methanol/dichloro-methane mixture in a ratio gradually ranging from 1:99 to 2:98) and then a second silica gel column purification was performed with a mobile phase consisting of a 95:5 ethyl acetate/hexane mixture, resulting in the pure title compound (319 mg, yield 62%) which was characterised by its mass spectrum as follows: MS (m/z): 499 ([M+H]⁺, 100).

### EXAMPLE 12

### Synthesis of 6-(3,4-dimethoxyphenyl)pyrido[3,2-d]pyrimidin-2(1H)-4(3H)-dione

To a solution of 2-carboxamido-3-amino-6-(3,4-dimethoxyphenyl)-pyridine (4.10 g, 15 mmol) in 1,4-dioxane (150 ml) was added triphosgene (2.22 g, 7.5 mmol). The solution was refluxed for 25 minutes and then evaporated to dryness. The crude compound was crystallized from acetic acid (150 ml) and washed with ethyl acetate, diethyl ether and dried under vacuum over P₂O₅, resulting in the pure title compound (3.60 g, yield 80%) which was characterised by its mass spectrum as follows: MS (m/z): 300 ([M+H]⁺, 100).

### EXAMPLE 13

### Synthesis of 2,4-dichloro-6-(3,4-dimethoxyphenyl)pyrido[3,2-d]pyrimidine

To a suspension of 6-(3,4-dimethoxyphenyl)pyrido[3,2-d]pyrimidin-2(1H)-4(3H)-dione (2.69 g, 9 mmol) in POC₃ (60 ml) was added triethylamine (3.47 ml). The reaction mixture was refluxed under nitrogen until completion. The reaction was cooled down to room temperature and evaporated to dryness. The residue was partitioned between water and dichloromethane. The organic layer was washed with brine. The combined organic layers were evaporated and the residue was purified by silica gel column chromatography (the mobile phase being a hexane/ethyl acetate mixture in a ratio 6:4), resulting in the pure title compound (yield 83%) which was characterised by its mass spectrum as follows: MS (m/z): 336, 338 ([M+H]⁺, 100).

### EXAMPLE 14

### Synthesis of 2-chloro-4-(4-[3-methylphenyl)amino]carbonyl]piperazin-1-yl)-6-(3,4-dimethoxyphenyl)pyrido[3,2-d]pyrimidine

To a suspension of 2,4-dichloro-6-(3,4-dimethoxyphenyl)pyrido[3,2-d]pyrimidine (672 mg, 2 mmol) in THF (10 ml) was added piperazine-1-carboxylic acid m-tolylamide (484 mg, 2.2 mmol) and triethylamine (10 mmol, 1.40 ml). The reaction mixture was stirred at room temperature for 10 minutes. The mixture was evaporated to dryness. The residue was redissolved in dichloromethane and extracted with brine. The combined organic layers were evaporated in vacuo and the crude residue was purified by silica gel column chromatography (the mobile phase being a hexane/ethyl acetate mixture in a ratio 1:1), resulting in the pure title compound (760 mg, yield 73%) which was characterised by its mass spectrum as follows: MS (m/z): 519, 521 ([M+H]⁺, 100).

### EXAMPLE 15

### Synthesis of 2-dimethylamino-4-(4-[3-methylphenyl)amino]carbonyl]piperazin-1-yl)-6-(3,4-dimethoxyphenyl)pyrido[3,2-d]pyrimidine

To a suspension of 2-chloro-4-(4-[3-methylphenyl)amino]carbonyl]piperazin-1-yl)-6-(3,4-dimethoxyphenyl)pyrido[3,2-d]pyrimidine (0.35 mmol, 181 mg) in dioxane (5 ml) was added dimethylamine (100 μl of a 40% solution in water). The reaction was stirred at 80° C. for 1.5 hours, after which an additional amount (100 μl) of the dimethylamine solution was added. The reaction was stirred for another 18 hours and then, cooled down, and diluted with dichloromethane (50 ml). The reaction mixture was extracted with a saturated sodium bicarbonate solution. The combined organic layers were evaporated in vacuo. The residue was purified by preparative thin layer chromatography on silica (the mobile phase being a hexane/ethyl acetate mixture in a ratio 1:9), resulting in the pure title compound (57 mg, yield 31%) which was characterised by its mass spectrum as follows: MS (m/z): 528 ([M+H]⁺, 100).

### EXAMPLE 16

### Synthesis of 2-[(N-hydroxyethyl)morpholino]-4-(4-[3-methylphenyl)amino]carbonyl]piperazin-1-yl)-6-(3,4-dimethoxyphenyl)pyrido[3,2-d]pyrimidine

N-(2-hydroxyethyl)morpholine (55 μl, 0.45 mmol) was dissolved in dry tetrahydrofuran (5 ml) and sodium hydride 60% (20 mg, 0.495 mmol) was added. The solution was stirred at 60° C. under nitrogen for 20 minutes and then, 2-chloro-4-(4-[3-methylphenyl)amino]carbonyl]piperazin-1-yl)-6-(3,4-dimethoxyphenyl)pyrido[3,2-d]pyrimidine (156 mg, 0.3 mmol) was added. The reaction mixture was stirred for 1 hour at 60° C. The mixture was cooled down to room temperature, diluted with brine and extracted with ethyl acetate. The combined organic layers were evaporated in vacuo and purified by preparative thin layer chromatography on silica (the mobile phase being a methanol/dichloromethane mixture in a ratio 7.5:92.5), resulting in the pure title compound (166 mg, yield 90%) which was characterised by its mass spectrum as follows: MS (m/z): 614 ([M+H]⁺, 100).

### EXAMPLE 17

### Synthesis of 2-(1-methyl-2-pyrrolidino-ethoxy)-4-(4-[3-methylphenyl)amino]carbonyl]piperazin-1-yl)-6-(3,4-dimethoxyphenyl)pyrido[3,2-d]pyrimidine

Sodium hydride 60% (20 mg, 0.495 mmol) was dissolved in dry tetrahydrofuran (5 ml) and 1-methyl-2-pyrrolidine-ethanol (62 μl, 0.45 mmol) was added. The mixture was refluxed under an N₂-atmosphere for 15 minutes. Then, 2-chloro-4-(4-[3-methylphenyl)amino]carbonyl]piperazin-1-yl)-6-(3,4-dimethoxyphenyl)pyrido[3,2-d]pyrimidine (156 mg, 0.30 mmol) was added and the reaction mixture was refluxed under nitrogen for 16 hours. The reaction mixture was diluted with distilled water and extracted three times with ethyl acetate. The combined organic extracts were washed with brine and dried over Na₂SO₄. Upon filtration and evaporation in vacuo, the crude product was purified by preparative thin layer chromatography on silica with a dichloromethane/methanol mixture (ratio 9:1) as the mobile phase to afford 79 mg (yield 43%) of the title compound which was characterised by its mass spectrum as follows: MS (m/z): 612 ([M+H]⁺, 100).

### EXAMPLE 18

### Synthesis of 2-(2-phenoxyethoxy)-4-(4-[3-methylphenyl)amino carbonyl]piperazin-1-yl)-6-(3,4-dimethoxyphenyl)pyrido[3,2-d]pyrimidine

Sodium hydride 60% (25 mg, 0.62 mmol) and 2-phenoxyethanol (63 mg, 0.45 mmol) were dissolved in dry tetrahydrofuran (5 ml). The reaction mixture was refluxed under a nitrogen atmosphere for 15 minutes. Then, 2-chloro-4-(4-[3-methylphenyl)amino]carbonyl]piperazin-1-yl)-6-(3,4-dimethoxyphenyl)pyrido[3,2-d]pyrimidine (156 mg, 0.30 mmol) was added and the reaction was refluxed under nitrogen for 3 hours. The reaction mixture was diluted with distilled water and extracted with dichloromethane. Combined organic extracts were dried over Na₂SO₄. Upon filtration and evaporation in vacuo, the crude product was purified by preparative thin layer chromatography on silica with a n-hexane/ethyl acetate mixture (ratio 1.5:1) as the mobile phase. Recrystallization from ethyl acetate afforded 124 mg (yield 67%) of the title compound which was characterised by its mass spectrum as follows: MS (m/z): 621 ([M+H]⁺, 100).

### EXAMPLE 19

### Synthesis of 2-phenyl-4-(4-[3-methylphenyl)amino]carbonyl]piperazin-1-yl)-6-(3,4-dimethoxyphenyl)pyrido[3,2-d]pyrimidine

A suspension of 2-chloro-4-(4-[3-methylphenyl)amino]carbonyl]piperazin-1-yl)-6-(3,4-dimethoxyphenyl)pyrido[3,2-d]pyrimidine (156 mg, 0.30 mmol), potassium carbonate (181 mg, 1.31 mmol) and phenylboronic acid (49 mg, 0.39 mmol) in 1,4-dioxane (4.5 ml) and water (1.5 ml) was purged with a stream of nitrogen gas for 10 minutes. Tetrakis(triphenylphosphine)palladium(0) (18 mg, 15.6 μmol) was added and the reaction mixture was refluxed under a nitrogen atmosphere for 30 minutes. Upon cooling, the mixture was diluted with ethyl acetate and washed twice with brine. The organic layer was dried over Na₂SO₄ and subsequently filtered and evaporated in vacuo. Recrystallization from ethyl acetate afforded 74 mg (yield 44%) of the title compound which was characterised by its mass spectrum as follows: MS (m/z): 561 ([M+H]⁺, 100).

### EXAMPLE 20

### Synthesis of 2-amino-6-chloropyrido[3,2-d]pyrimidin-4(3H)-one

2,4-diamino-6-chloropyrido[3,2-d]pyrimidine (7.5 g, 38 mmole), e.g. prepared according to Colbry et al., J. Heterocycl. Chem. (1984) 21:1521, was suspended in 6 N HCl (300 ml) and the mixture was refluxed for 5 hours. After cooling, the pH was made alkaline (pH about 9-10) by means of 10 N NaOH. The precipitate obtained was filtered, washed with H₂O and dried at 100° C., resulting in the pure title compound (7.0 g, yield 95%) which was characterized by its mass spectrum as follows: MS (m/z): 197 ([M+H]⁺, 100).

### EXAMPLE 21

### Synthesis of 2-amino-6-(3,4-dimethoxyphenyl)-pyrido[3,2-d]pyrimidin-4(3H)-one

To a degassed suspension of 2-amino-6-chloro-pyrido[3,2-d]pyrimidin-4(3H)-one (7.30 g, 37 mmole), 3,4-dimethoxyphenyl boronic acid (7.50 g, 40 mmole) and potassium carbonate (20.70 g, 152 mmole) in a mixture of dioxane (540 ml) and H₂O (120 ml), was added a catalytic amount of tetrakis(triphenylphosphine)palladium(0) (2.16 g, 18.5 mmole). The mixture was refluxed for 24 hours and, after cooling at room temperature, was filtered. The filtrate was acidified with 5 N HCl to pH 4 and the resulting precipitate was filtered and then washed successively with H₂O, ethanol and diethylether, and dried under vacuum resulting in the pure title compound (8.0 g, yield 73%) which was characterized by its mass spectrum as follows: MS (m/z): 299 ([M+H]⁺, 100).

### EXAMPLE 22

### Synthesis of 2-acetamido-6-(3,4-dimethoxyphenyl)pyrido[3,2-d]pyrimidin-4(3H)-one

2-amino-6-(3,4-dimethoxyphenyl)-pyrido[3,2-d]pyrimidin-4(3H)-one (2.0 g, 6.70 mmole) was suspended in acetic anhydride (180 ml) and acetic acid (20 ml) and the mixture was refluxed for 16 hours. The hot suspension was filtered and the filtrate was concentrated under reduced pressure until crystallization started. The precipitate was filtered off to give the pure title compound (1.76 g, yield 77%) which was characterized by its mass spectrum as follows: MS (m/z): 341 ([M+H]⁺, 100).

### EXAMPLE 23

### Synthesis of 2-acetamido-4-(1,2,4-triazolyl)-6-(3,4-dimethoxyphenyl)-Pyrido[3,2-d]pyrimidine

A suspension of 1,2,4-triazole (8.28 g, 120 mmole) and phosphorus oxychloride (3.2 ml, 36 mmol) in dry acetonitrile (150 ml) was added to a stirred suspension of 2-acetamido-6-(3,4-dimethoxyphenyl)pyrido[3,2-d]pyrimidin-4(3H)-one (4.08 g, 12 mmole) and triethylamine (5.2 ml, 36 mmole) in dry acetonitrile (150 ml). The mixture was stirred at room temperature under nitrogen for 3 days and the yellow precipitate was filtered off, then successively washed with ethanol and ether, and dried over P₂O₅ in a vacuum dessicator resulting in the pure title compound (4.3 g, yield 90%) which was characterized by its mass spectrum as follows: MS (m/z): 392 ([M+H]⁺, 100), 414 ([M+Na]⁺; 804 [2M+Na]⁺

### EXAMPLES 24 AND 25

### Synthesis of 2-amino-6-(3,4-dimethoxyphenyl)-4-alkoxy-pyrido[3,2-d]pyrimidines

Sodium (44 mg, 2 mmol) was suspended in a suitable alcohol (10 ml) and the solution was warmed up to 50° C. until the sodium dissolved completely. Then, 2-acetamido-4-(1,2,4-triazolyl)-6-(3,4-dimethoxyphenyl)-pyrido[3,2-d]pyrimidine (160 mg, 0.4 mmole) was added and the mixture was stirred at room temperature for 16 hours. The mixture was then neutralized with a solution of 1 N HCl and the volatiles were removed under reduced pressure. The crude mixture was purified by silica gel column chromatography, the mobile phase consisting of CH₃OH/CH₂Cl₂ mixtures (in a ratio gradually ranging from 2:98 to 10:90), thus providing the desired compound with yields ranging from 40 to 60%, depending upon the alcohol used. The following compounds were made according to this procedure:
2-amino-4-isopropoxy-6-(3,4-dimethoxyphenyl)pyrido[3,2-o]pyrimidine (example 44) was obtained from isopropyl alcohol and characterized by its mass spectrum as follows: MS (m/z): 341 ([M+H]⁺, 100), and
2-amino-4-(2-phenoxyethoxy)-6-(3,4-dimethoxyphenyl)-pyrido[3,2-d]pyrimidine (example 45) was obtained from 2-phenoxyethanol and characterized by its mass spectrum as follows: MS (m/z): 419 ([M+H]⁺, 100).

### EXAMPLES 26 TO 36

### Synthesis of 2-acetylamino-4-alkylamino-6-(3,4-dimethoxy-phenyl)pyrido[3,2-d]pyrimidines, 2-acetylamino-4-cycloalkylamino-6-(3,4-dimethoxy-phenyl)pyrido[3,2-d]pyrimidines, 2-acetylamino-4-heteroarylalkylamino-6-(3,4-dimethoxyphenyl)pyrido[3,2-d]pyrimidines, 2-acetylamino-4-arylamino-6-(3,4-dimethoxyphenyl)pyrido[3,2-d]pyrimidines and 2-acetylamino-4-heterocyclic amino-6-(3,4-dimethoxyphenyl)pyrido[3,2-d]pyrimidines and the corresponding 2-amino-4-alkylamino-6-(3,4-dimethoxyphenyl)pyrido[3,2-d]pyrimidines, 2-amino-4-cycloalkyl-amino-6-(3,4-dimethoxyphenyl)pyrido[3,2-d]pyrimidines, 2-amino-4-heteroarylalkyl-amino-6-(3,4-dimethoxyphenyl)pyrido[3,2-d]pyrimidines, 2-amino-4-arylamino-6-(3,4-dimethoxyphenyl)pyrido[3,2-d]pyrimidines and 2-amino-4-heterocyclic amino-6-(3,4-dimethoxyphenyl)pyrido[3,2-d]pyrimidines

A suitable alkylamine, cycloalkylamine, arylamine, heterocyclic amine or heteroarylalkylamine (2 equivalents, 0.8 mmole) was added to a stirred suspension of 2-acetamido-4-(1,2,4-triazolyl)-6-(3,4-dimethoxyphenyl)pyrido[3,2-d]pyrimidine (160 mg, 0.4 mmole) in dioxane. The mixture was heated at 50° C. for 24 hours and the volatiles were removed under reduced pressure, yielding a crude 2-acetylamino-4-alkylamino-6-(3,4-dimethoxyphenyl)pyrido[3,2-d]pyrimidine, 2-acetylamino-4-cyclo-alkylamino-6-(3,4-dimethoxyphenyl)pyrido[3,2-d]pyrimidine, 2-acetylamino-4-hetero-arylalkylamino-6-(3,4-dimethoxyphenyl)pyrido[3,2-d]pyrimidine, 2-acetylamino-4-aryl-amino-6-(3,4-dimethoxyphenyl)pyrido[3,2-d]pyrimidine or 2-acetylamino-4-hetero-cyclic amino-6-(3,4-dimethoxyphenyl)pyrido[3,2-d]pyrimidine as an intermediate. This crude residue was resuspended in a 0.2 N sodium ethoxide (20 ml) and the mixture was stirred at room temperature for 24 hours and neutralized with 5-6 N HCl in isopropyl alcohol, yielding the crude corresponding 2-amino-4-alkylamino-6-(3,4-dimethoxyphenyl)pyrido[3,2-d]pyrimidine, 2-amino-4-cycloalkylamino-6-(3,4-dimethoxyphenyl)pyrido[3,2-d]pyrimidine, 2-amino-4-heteroarylalkylamino-6-(3,4-dimethoxyphenyl)pyrido[3,2-d]pyrimidine, 2-amino-4-arylamino-6-(3,4-dimethoxy-phenyl)pyrido[3,2-d]pyrimidine or 2-amino-4-heterocyclic amino-6-(3,4-dimethoxy-phenyl)pyrido[3,2-d]pyrimidine as the final compound. This crude residue was purified by silica gel column chromatography, the mobile phase consisting of CH₃OH/CH₂Cl₂ mixtures (in a ratio gradually ranging from 2:98 to 10:90) with 0.5% concentrated ammonia if needed. This procedure provided the desired final compounds with yields ranging from 40 to 80%. The following final compounds were synthesized according to this procedure (each time through the corresponding intermediate having the 2-amino group protected in the form of acetamido):
2-amino-4-[4-(ethoxycarbonyl)piperidin-1-yl]-6-(3,4-dimethoxyphenyl)pyrido[3,2-d]pyrimidine (example 26) was obtained from ethyl isonipecotate and characterized by its mass spectrum as follows: MS (m/z): 438 ([M+H]⁺, 100),
2-amino-4-(3-methyl-anilino)-6-(3,4-dimethoxyphenyl)-pyrido[3,2-d]pyrimidine (example 27) was obtained from 3-methyl-aniline and characterized by its mass spectrum as follows: MS (m/z): 388 ([M+H]⁺, 100),
2-amino-4-[3,4-(methylenedioxy)aniline]-6-(3,4-dimethoxyphenyl)pyrido[3,2-d]pyrimidine (example 28) was obtained from 3,4-(methylenedioxy)aniline and characterized by its mass spectrum as follows: MS (m/z): 418 ([M+H]⁺, 100),
2-amino-4-(3-bromo-anilino)-6-(3,4-dimethoxyphenyl)pyrido[3,2-d]-pyrimidine (example 29) was obtained from 3-bromo-aniline and characterized by its mass spectrum as follows: MS (m/z): 452 ([M+H]⁺, 100),
2-amino-4-(2-chloro-5-methoxy-anilino)-6-(3,4-dimethoxyphenyl)pyrido[3,2-d]pyrimidine (example 30) was obtained from 2-chloro-5-methoxy-aniline and characterized by its mass spectrum as follows: MS (m/z): 438 ([M+H]⁺, 100),
2-amino-4-(N-methyl-piperazino)-6-(3,4-dimethoxyphenyl)-pyrido[3,2-d]pyrimidine (example 31) was obtained from N-methyl-piperazine and characterized by its mass spectrum as follows: MS (m/z): 381 ([M+H]⁺, 100),
2-amino-4-(thienyl-2-methylamino)-6-(3,4-dimethoxyphenyl)-pyrido[3,2-d]pyrimidine-2,4-diamine (example 32) was obtained from 2-thiophenylmethylamine and characterized by its mass spectrum as follows: MS (m/z): 394 ([M+H]⁺, 100),
2-amino-4-[4-(2-aminoethyl)morpholino]-6-(3,4-dimethoxyphenyl)-pyrido[3,2-d]pyrimidine (example 33) was obtained from 4-(2-aminoethyl)morpholine and characterized by its mass spectrum as follows: MS (m/z) 411 ([M+H]⁺, 100),
2-amino-4-(2,2-dimethoxyethylamino)-6-(3,4-dimethoxyphenyl)pyrido[3,2-d]pyrimidine (example 34) was obtained from 2,2-dimethoxyethylamine and characterized by its mass spectrum as follows: MS (m/z): 386 ([M+H]⁺, 100),
2-amino-4-[2-(aminomethyl)pyridino]-6-(3,4-dimethoxyphenyl)-pyrido[3,2-d]pyrimidine (example 35) was obtained from 2-(aminomethyl)pyridine and characterized by its mass spectrum as follows: MS (m/z): 389 ([M+H]⁺, 100), and
2-amino-4-(1,4-diaminocyclohexyl)-6-(3,4-dimethoxyphenyl)pyrido[3,2-d]pyrimidine (example 36) was obtained from trans-1,4-diaminocyclohexane and characterized by its mass spectrum as follows: MS (m/z): 395 ([M+H]⁺, 100).

### EXAMPLE 37

### Synthesis of 6-(3,4-dimethoxyphenyl)-3-nitropyridine-2-carbonitrile

To a degassed suspension of 6-chloro-2-cyano-3-nitropyridine (5.51 g, 30 mmole), 3,4-dimethoxyphenyl boronic acid (6.55 g, 36 mmole) and potassium carbonate (16.59 g, 120 mmole) in dry toluene (300 ml), was added a catalytic amount of tetrakis(triphenylphosphine)palladium (3.47 g, 3 mmole). The mixture was refluxed for 24 hours and after cooling, the volatiles were evaporated to dryness. The crude mixture was purified by silica gel column chromatography, the mobile phase consisting of hexane/CH₂Cl₂ mixtures (in a ratio gradually ranging from 15:85 to 0:100). The appropriated fractions were collected, evaporated to dryness and the residue was suspended in ether. The orange precipitate was filtered off, washed with ether and dried, resulting in the pure title compound (6.79 g, yield 79%).

### EXAMPLE 38

### Synthesis of 3-amino-6-(3,4-dimethoxyphenyl)pyridine-2-carbonitrile

Iron (7.14 g, 128 mmole) was added portionwise to a stirred suspension of 6-(3,4-dimethoxyphenyl)-3-nitropyridine-2-carbonitrile (4.56 g; 16 mmole) in methanol (80 ml) and 37% HCl (25 ml). The mixture was refluxed for 5 hours and, after cooling, the pH was adjusted to 9-10 by means of concentrated ammonium hydroxide (30 ml). The mixture was filtered over Celite and washed with MeOH and EtOAc. The filtrate was evaporated to dryness and the residue was purified on silica gel column chromatography, using a mixture of CH₂Cl₂/EtOAc (in a ratio of 95:5) as eluent, to obtain the pure title compound (2.62 g, yield 64%) which was characterized by its mass spectrum as follows: MS (m/z): 256 ([M+H]⁺, 100).

### EXAMPLE 39

### Synthesis of 2,4-diamino-6-(3,4-dimethoxyphenyl)pyrido[3,2-d]pyrimidine

A solution of sodium (423 mg, 18.4 mmole) in n-butanol (180 ml) was added to 3-amino-6-(3,4-dimethoxyphenyl)pyridine-2-carbonitrile (2.36 g; 9.20 mmole) and guanidine hydrochloride (1.76 g; 18.4 mmole). The mixture was refluxed for 4 hours and, after cooling, the solvent was evaporated under reduced pressure. The residue was purified on silica gel column chromatography, using a mixture of CH₂Cl₂/MeOH (in a ratio of 95:5) as eluent, resulting in the pure title compound (1.88 g; yield 69%) which was characterized by its mass spectrum as follows: MS (m/z): 298 ([M+H]⁺, 100).

### EXAMPLE 40

### Synthesis of 2-amino-6-(3,4-dimethoxyphenyl)pyrido[3,2-d]pyrimidin-4(3H)-one hydrochloride

2,4-diamino-6-(3,4-dimethoxyphenyl)pyrido[3,2-d]pyrimidine (1.27 g, 4.27 mmole) was suspended in 6 N HCl (85 ml) and the mixture was refluxed for 8 hours. After cooling, the precipitate was filtered off, washed with H₂O and dried over P₂O₅ and KOH, resulting in the pure title compound (1.29 g; yield 90%) which was characterized by its mass spectrum as follows: MS (m/z): 299 ([M+H]⁺, 100)

### EXAMPLE 41

### Synthesis of 2-amino-4-morpholino-6-(3,4-dimethoxyphenyl)-pyrido[3,2-d]pyrimidine

2-amino-6-(3,4-dimethoxyphenyl)pyrido[3,2-d]pyrimidin-4(3H)-one hydrochloride (332 mg; 1 mmole) was suspended in toluene (10 ml) with a catalytic amount of p-toluenesulfonic acid and ammonium sulfate. Then, 1,1,1,3,3,3-hexamethyldisilizane (3.2 ml; 15 mmole) and morpholine (0.53 ml; 6 mmol) were added. The mixture was refluxed for 24 hours and evaporated to dryness. The residue was purified by silica gel column chromatography, using a mixture of CH₂Cl₂/MeOH: 96:4 as eluent, resulting in the pure title compound (120 mg; yield 32%) which was characterized by its mass spectrum as follows: MS (m/z): 368 ([M+H]⁺, 100).

### EXAMPLE 42

### Synthesis of 2-amino-4-(4-{[(3-methylphenyl)amino]carbonyl}piperazin-1-yl)-6-(3,4-dimethoxyphenyl)-pyrido[3,2-d]pyrimidine

Piperazine (258 mg; 3 mmole) was added to a stirred suspension of 2-acetamido-6-(3,4-dimethoxyphenyl)-4-(1,2,4-triazolyl)pyrido[3,2-d]pyrimidine (586 mg; 1.5 mmole) in dioxane (50 ml). The mixture was stirred at room temperature for 24 hours and the volatiles were removed under reduced pressure, yielding 2-acetamido-4-(N-piperazinyl)-6-(3,4-dimethoxyphenyl)-pyrido[3,2-d]pyrimidine as a crude residue. The latter was dissolved in DMF and m-tolyl isocyanate (0.66 ml, 5 mmole) was added. After 18 hours at room temperature, the solvent was removed and the residue was suspended in a mixture of CH₂Cl₂ (20 ml) and sodium ethoxide 0.2 N (20 ml). The suspension was stirred during 16 hours and neutralized with 5-6 N HCl in isopropyl alcohol. The crude residue was purified by silica gel column chromatography, the mobile phase consisting of a CH₃OH/CH₂Cl₂ mixture in a ratio gradually ranging from 2:98 to 5:95, thus resulting in the pure title compound (350 mg, yield 43%) which was characterized by its mass spectrum as follows: MS (m/z): 542 ([M+H]⁺, 100).

### EXAMPLE 43

### Synthesis of 2-amino-4-(4-fluorophenyl-piperazin-1-yl)-6-(3,4-dimethoxyphenyl)-pyrido[3,2-d]pyrimidine

1-(4-fluorophenyl)-piperazine (90 mg, 0.5 mmole) was added to a stirred suspension of 2-acetamido-6-(3,4-dimethoxyphenyl)-4-(1,2,4-triazolyl)pyrido[3,2-d]pyrimidine (120 mg, 0.3 mmole) in dioxane (10 ml). The mixture was stirred at 60° C. for 48 hours and the volatiles were removed under reduced pressure, yielding the crude 2-acetamido-4-(4-fluorophenyl-piperazin-1-yl-)-6-(3,4-dimethoxyphenyl)-pyrido[3,2-d]pyrimidine. The latter was dissolved in a mixture of CH₂Cl₂ (20 ml) and sodium ethoxide 0.2 N (20 ml). The suspension was stirred during 16 hours and neutralized with 5-6 N HCl in isopropyl alcohol. The crude residue was purified by preparative thin layer chromatography, the mobile phase consisting of a CH₃OH/CH₂Cl₂ mixture in a ratio of 5:95, resulting in the pure title compound (40 mg, yield 29%) which was characterized by its mass spectrum as follows: MS (m/z): 461 ([M+H]⁺, 100).

### EXAMPLE 44

### Synthesis of 2-amino-4-(4-methylphenyl-piperazin-1-yl)-6-(3,4-dimethoxyphenyl)-pyrido[3,2-d]pyrimidine

A similar procedure as in example 43 was used but starting from 1-(4-methylphenyl)-piperazine and resulted, through the corresponding 2-acetamido intermediate, in the pure title compound (49% yield) which was characterized by its mass spectrum as follows: MS (m/z): 457 ([M+H]⁺, 100).

### EXAMPLE 45

### Synthesis of 2-amino-4-(phenoxy-ethyl-piperazin-1-yl)-6-(3,4-dimethoxyphenyl)-pyrido[3,2-d]pyrimidine

A similar procedure as in example 43 was used but starting from 1-(2-phenoxy-ethyl)-piperazine and resulted, through the corresponding 2-acetamido intermediate, in the pure title compound (56% yield) which was characterized by its mass spectrum as follows: MS (m/z): 488 ([M+H]⁺, 100).

### EXAMPLE 46

### Synthesis of 2-amino-4-(3-chlorophenyl-piperazin-1-yl)-6-(3,4-dimethoxyphenyl)-pyrido[3,2-d]pyrimidine

A similar procedure as in example 43 was used but starting from 1-(3-chlorophenyl)-piperazine and resulted, through the corresponding 2-acetamido intermediate, in the pure title compound (42% yield) which was characterized by its mass spectrum as follows: MS (m/z) 478 ([M+H]⁺, 100)

### EXAMPLE 47

### Synthesis of 2-amino-4-(2-pyridyl-piperazin-1-yl)-6-(3,4-dimethoxy-phenyl)-pyrido[3,2-d]pyrimidine

A similar procedure as in example 43 was used but starting from 1-(2-pyridyl)-piperazine and resulted, through the corresponding 2-acetamido intermediate, in the pure title compound (37% yield) which was characterized by its mass spectrum as follows: MS (m/z): 444 ([M+H]⁺, 100).

### EXAMPLE 48

### Synthesis of 2-amino-4-[2-(piperazin-1-yl)-acetic acid N-(2-thiazolyl)-amide]-6-(3,4-dimethoxyphenyl)-pyrido[3,2-d]pyrimidine

A similar procedure as in example 43 was used but starting from 2-(piperazin-1-yl)-acetic acid N-(2-thiazolyl)-amide and resulted, through the corresponding 2-acetamido intermediate, in the pure title compound (52% yield) which was characterized by its mass spectrum as follows: MS (m/z): 507 ([M+H]⁺, 100).

### EXAMPLE 49

### Synthesis of 2-amino-4-(N-acetyl-piperazinyl)-6-(3,4-dimethoxyphenyl)-Pyrido[3,2-d]pyrimidine

A similar procedure as in example 43 was used but starting from N-acetyl-piperazine and resulted, through the corresponding 2-acetamido intermediate, in the pure title compound (33% yield) which was characterized by its mass spectrum as follows: MS (m/z): 409 ([M+H]⁺, 100).

### EXAMPLE 50

### Synthesis of 2-amino-4-(1-piperonyl-piperazin-1-yl)-6-(3,4-dimethoxy-phenyl)-pyrido[3,2-d]pyrimidine

A similar procedure as in example 43 was used but starting from 1-piperonyl-piperazine and resulted, through the corresponding 2-acetamido intermediate, in the pure title compound (38% yield) which was characterized by its mass spectrum as follows: MS (m/z): 501 ([M+H]⁺, 100).

### EXAMPLE 51

### Synthesis of 2-amino-4-[1-(2-furoyl)-piperazin-1-yl]-6-(3,4-dimethoxy-phenyl)-pyrido[3,2-d]pyrimidine

A similar procedure as in example 43 was used but starting from 1-(2-furoyl)-piperazine instead of 1-(4-fluorophenyl)-piperazine and resulted, through the corresponding 2-acetamido intermediate, in the pure title compound which was characterized by its mass spectrum as follows: MS (m/z): 461 ([M+H]⁺, 100).

### EXAMPLE 52

### Synthesis of 2-amino-4-(1-benzylpiperazin-1-yl)-6-(3,4-dimethoxyphenyl)-pyrido[3,2-d]pyrimidine

A similar procedure as in example 43 was used but starting from 1-benzylpiperazine and resulted, through the corresponding 2-acetamido intermediate, in the pure title compound (39% yield) which was characterized by its mass spectrum as follows: MS (m/z): 457 ([M+H]⁺, 100).

### EXAMPLE 53

### Synthesis of 2-acetamido-4-(piperazin-1-yl)-6-(3,4-dimethoxyphenyl)-pyrido[3,2-o]pyrimidine

Piperazine (430 mg, 5 mmole) was added to a stirred suspension of 2-acetamido-6-(3,4-dimethoxyphenyl)-4-(1,2,4-triazolyl)pyrido[3,2-d]pyrimidine (977 mg, 2.5 mmole) in dioxane (70 ml). The reaction mixture was refluxedfor 16 hours. The precipitate was filtered off and washed with a small amount of dioxane. The filtrate was evaporated to dryness and the residue washed with diethyl ether. Both fractions (the precipate and the washed filtrate) were combined, resulting in the pure title compound (805 mg, yield 79%) which was characterized by its mass spectrum as follows: MS (m/z): 409 ([M+H]⁺, 100).

### EXAMPLES 54 TO 58

### Synthesis of 2-amino-4-(N-carbamoyl-piperazin-1-yl)-6-(3,4-dimethoxy-phenyl)-pyrido[3,2-d]pyrimidines

To a solution of 2-acetamido-4-(piperazin-1-yl)-6-(3,4-dimethoxyphenyl)-pyrido[3,2-d]pyrimidine (200 mg, 0.5 mmole) in DMF (5 ml) was added a suitable isocyanate (0.75 mmole). The reaction mixture was stirred for 16 hours at room temperature. The solvents were evaporated in vacuo yielding a crude 2-acetamido-4-(N-carbamoyl-piperazin-1-yl)-6-(3,4-dimethoxyphenyl)-pyrido[3,2-d]pyrimidine as an intermediate. This crude residue was dissolved in a mixture of CH₂Cl₂ (10 ml) and sodium ethoxide 0.2 N (10 ml), the resulting suspension was stirred for 16 hours and neutralized with 5-6 N HCl in isopropyl alcohol, yielding a crude 2-amino-4-(N-carbamoyl-piperazin-1-yl)-6-(3,4-dimethoxyphenyl)-pyrido[3,2-d]pyrimidine as the final compound. This crude product was purified by preparative thin layer chromatography on silica, the mobile phase consisting of a CH₃OH/CH₂Cl₂ mixture in a ratio of 10:90, resulting in the pure desired compounds in yields varying from 20 to 40%, depending upon the isocyanate used. The following final compounds were synthesized according to this procedure (each time through the corresponding intermediate having the 2-amino group protected in the form of acetamido):
2-amino-4(N-3-thienylcarbamoyl-piperazin-1-yl)-6-(3,4-dimethoxyphenyl)-pyrido[3,2-d]pyrimidine (example 54) was obtained from 3-thienyl isocyanate and characterized by its mass spectrum as follows: MS (m/z): 492 ([M+H]⁺, 100),
2-amino-4(N-2,6-dichloro-pyridinyl-carbamoyl-piperazin-1-yl)-6-(3,4-dimethoxyphenyl)-pyrido[3,2-d]pyrimidine (example 55) was obtained from 2,6-dichloro-4-isocyanate-pyridine and was characterized by its mass spectrum as follows: MS (m/z): 555, 557 ([M+H]⁺, 100),
2-amino-4(N-4-fluoro-phenyl-carbamoyl-piperazin-1-yl)-6-(3,4-dimethoxy-phenyl)-pyrido[3,2-d]pyrimidine (example 56) was obtained from 4-fluoro-phenyl isocyanate and was characterized by its mass spectrum as follows: MS (m/z): 504 ([M+H]⁺, 100),
2-amino-4(N-3-chloro-4-fluoro-phenyl-carbamoyl-piperazin-1-yl)-6-(3,4-dimethoxyphenyl)-pyrido[3,2-d]pyrimidine (example 57) was obtained from 3-chloro-4-fluoro-phenyl isocyanate and was characterized by its mass spectrum as follows: MS (m/z): 539 ([M+H]⁺, 100), and
2-amino-4(N-3-chloro-phenyl-carbamoyl-piperazin-1-yl)-6-(3,4-dimethoxy-phenyl)-pyrido[3,2-d]pyrimidine (example 58) was obtained from 3-chloro-phenyl isocyanate and was characterized by its mass spectrum as follows: MS (m/z): 521 ([M+H]⁺, 100).

### EXAMPLE 59

### Synthesis of 2-amino-4-[(N-4-chloro-phenoxy-acetyl)-piperazin-1-yl]-6-(3,4-dimethoxyphenyl)-pyrido[3,2-d]pyrimidine

To a solution of 2-acetamido-4-(piperazin-1-yl)-6-(3,4-dimethoxyphenyl)-pyrido[3,2-d]pyrimidine (200 mg, 0.5 mmole) in dioxane (15 ml) was added p-chloro-phenoxy acetyl chloride (0.75 mmol). The reaction mixture was stirred for 16 hours at 50° C. overnight. The solvents were evaporated in vacuo yielding crude 2-acetamido-4-[(N-4-chloro-phenoxy-acetyl)-piperazin-1-yl]-6-(3,4-dimethoxyphenyl)-pyrido[3,2-d]pyrimidine as an intermediate. This crude residue was dissolved in a mixture of CH₂Cl₂ (10 ml) and sodium ethoxide 0.2 N (10 ml). The suspension was stirred for 16 hours and neutralized with 5-6 N HCl in isopropyl alcohol, yielding crude 2-amino-4-[(N-4-chloro-phenoxy-acetyl)-piperazin-1-yl]-6-(3,4-dimethoxyphenyl)-pyrido[3,2-d]pyrimidine as the final compound. This crude product was purified by preparative thin layer chromatography on silica, the mobile phase consisting of a CH₃OH/CH₂Cl₂ mixture in a ratio of 10:90, resulting in the pure title compound (98 mg, yield 37%) which was characterized by its mass spectrum as follows: MS (m/z): 536 ([M+H]⁺, 100).

### EXAMPLE 60

### Synthesis of 2-amino-4-[(N-phenoxy-acetyl)-piperazin-1-yl]-6-(3,4-dimethoxyphenyl)-pyrido[3,2-d]pyrimidine

A similar procedure as described in example 59 was performed, but using phenoxy acetyl chloride instead of p-chloro-phenoxy acetyl chloride and resulted, through the corresponding 2-acetamido intermediate, in the pure title compound which was characterized by its mass spectrum as follows: MS (m/z): 501 ([M+H]⁺, 100).

### EXAMPLE 61

### Synthesis of 3-amino-6-chloro-pyridine-2-carboxamide

To a suspension of 6-chloro-3-nitro-pyridine-2-carbonitrile (9.2 g, 50 mmole) in water (100 ml), was added 20 ml of a 25% ammonia aqueous solution. The mixture was stirred at room temperature for 20 minutes. Then, Na₂S₂O₄ (50 g, 86%, 150 mmole) was added portionwise, and the mixture was stirred at room temperature for another 2 hours. The precipitate formed was collected by filtration, washed two times with cold water (10 ml) and then dried over P₂O₅, resulting in the title compound (7.0 g, yield 81%) as a yellowish solid which was characterized by its mass spectrum as follows: MS (m/z): 172.1 ([M+H]⁺, 100).

### EXAMPLE 62

### Synthesis of 3-amino-5-chloro-pyridine-2-carboxamide

This compound was synthesized, by using the procedure of example 61 but from 5-chloro-3-nitro-pyridine-2-carbonitrile as a starting material, in 80% yield as a yellowish solid which was characterized by its mass spectrum as follows: MS (m/z): 172.1 ([M+H]⁺, 100).

### EXAMPLE 63

### Synthesis of 7-chloro-pyrido[3,2-d]pyrimidin-4(3H)one

A suspension of 3-amino-5-chloro-pyridine-2-carboxamide (3.43 g, 20 mmole) in triethyl orthoformate (50 ml) was refluxed for 3 hours. After cooling to room temperature, the precipitate was collected by filtration and washed with hexane. The title compound was obtained as a white solid (3.4 g, yield 94%) which was characterized by its mass spectrum as follows: MS (m/z): 182.1 ([M+H]⁺, 100).

### EXAMPLE 64

### Synthesis of 4,6-dichloro-pyrido[3,2-d]pyrimidine

To a mixture of 6-chloro-pyrido[3,2-d]pyrimidin-4(3H)one (3.0 g, 16.5 mmole) and N,N-diisopropylethylamine (9 ml, 50 mmole) in toluene (150 ml), was added POCl₃ (4.7 ml, 50 mmol). The resulting reaction mixture was refluxed for 1.5 hour. After cooling to room temperature, the solvent was removed under reduced pressure. The residue was dissolved in dichloromethane (200 ml) and washed with cold water till pH=6-7. The organic phase was dried over MgSO₄, filtrated and concentrated under reduced pressure to yield crude 4,6-dichloro-pyrido[3,2-d]pyrimidine which was not purified but used as such for further reactions.

### EXAMPLE 65

### Synthesis of 4-(piperazin-1-yl)-6-chloro-pyrido[3,2-d]pyrimidine

To a solution of piperazine (7.0 g) in 1,4-dioxane (100 ml) was added a solution of crude 4,6-dichloro-pyrido[3,2-d]pyrimidine in 1,4-dioxane (50 ml). The resulting mixture was stirred at room temperature for 1 hour. After concentration under reduced pressure, the residue was purified by silica gel flash chromatography, the mobile phase being a methanol/dichloromethane mixture (in a ratio gradually ragning from 1:10 to 1:5), resulting in the pure title compound as a yellowish solid (3.1 g, yield 76%) which was characterized by its mass spectrum as follows: MS (m/z): 250.1 ([M+H]⁺, 100).

### EXAMPLE 66

### Synthesis of 4,7-dichloro-pyrido[3,2-d]pyrimidine

This compound was synthesized from 7-chloro-pyrido[3,2-d]pyrimidin-4(3H)one using the procedure mentioned in example 64.

### EXAMPLE 67

### Synthesis of 7-chloro-4-(piperazin-1-yl)-pyrido[3,2-d]pyrimidine

The title compound was synthesized in 72% yield from 4,7-dichloro-pyrido[3,2-d]pyrimidine by the procedure of example 65 and was characterized by its mass spectrum as follows: MS (m/z): 250.1 ([M+H]⁺, 100).

### EXAMPLE 68

### Synthesis of 4-morpholino-6-chloro-pyrido[3,2-d]pyrimidine

The title compound was synthesized in 71% yield from 4,6-dichloro-pyrido[3,2-d]pyrimidine and morpholine by the procedure of example 65, and was characterized by its mass spectrum as follows: MS (m/z): 251.1 ([M+H]⁺, 100).

### EXAMPLE 69

### Synthesis of 4-[(N-3-chlorophenylcarbamoyl)-piperazin-1-yl]-7-chloro-pyrido[3,2-d]pyrimidine

To a solution of 4-(piperazin-1-yl)-7-chloro-pyrido[3,2-d]pyrimidine (1.0 g, 4 mmole) in dichloromethane (40 ml), was added 3-chlorophenyl isocyanate (615 mg, 4 mmole). The reaction mixture was stirred at room temperature for 1 hour. The solvent was removed under reduced pressure, resulting in the pure title compound (1.6 g, yield 99%) as a white solid which was characterized by its mass spectrum as follows: MS (m/z): 403.1 ([M+H]⁺, 100).

### EXAMPLE 70

### Synthesis of 4-[(N-3-chlorophenylcarbamoyl)-piperazin-1-yl]-6-chloro-pyrido[3,2-d]pyrimidine

This compound was synthesized from 4-(piperazin-1-yl)-6-chloro-pyrido[3,2-d]pyrimidine (2.5 g, 10 mmole) and 3-chlorophenyl isocyanate (1.54 g, 10 mmole) using the procedure of example 69, resulting in the pure title compound (4.0 g, 99%) as a white solid which was characterized by its mass spectrum as follows: MS (m/z): 403.1 ([M+H]⁺, 100).

### EXAMPLES 71 TO 78

### Synthesis of 4-[(N-3-chlorophenylcarbamoyl)-piperazin-1-yl]-7-aryl-pyrido[3,2-d]pyrimidines

To a solution of 4-[(N-3-chlorophenylcarbamoyl)-piperazin-1-yl]-7-chloro-pyrido[3,2-d]pyrimidine (0.5 mmole) in dioxane (20 ml) and water (5 ml) was added an appropriate arylboronic acid (0.5 mmole), K₂CO₃ (1.5 mmole), and tetrakis (triphenylphosphine)palladium(0) (0.025 mmole). The mixture was heated at 95° C. until the starting materials disappeared on thin layer chromatography. The reaction mixture was diluted with CH₂Cl₂ (50 ml) and washed with a 0.5 M Na₂CO₃ solution (10 ml), and the organic phase was concentrated under reduced pressure. The residue was purified by silica gel column chromatography, the mobile phase being an acetone/dichloromethane mixture (in a ratio gradually ranging from 1:3 to 1:2), resulting in the pure following compounds:
4-[(N-3-chlorophenylcarbamoyl)-piperazin-1-yl]-7-(3-chloro-4-methoxyphenyl)-pyrido[3,2-d]pyrimidine (example 71) was obtained from 3-chloro-4-methoxy-phenyl boronic acid (yield 81%) as a white solid which was characterized by its mass spectrum as follows: MS (m/z): 509.1 ([M+H]⁺, 100),
4-[(N-3-chlorophenylcarbamoyl)-piperazin-1-yl]-7-(3,4-dimethylphenyl)-pyrido[3,2-d]pyrimidine (example 72) was obtained from 3,4-dimethylphenyl boronic acid (yield 80%) as a white solid which was characterized by its mass spectrum as follows: MS (m/z): 473.2 ([M+H]⁺, 100),
4-[(N-3-chlorophenylcarbamoyl)-piperazin-1-yl]-7-(3,4-dichlorophenyl)-pyrido[3,2-d]pyrimidine (example 73) was obtained from 3,4-dichlorophenyl boronic acid (yield 82%) as a white solid which was characterized by its mass spectrum as follows: MS (m/z): 515.1 ([M+H]⁺, 100),
4-[(N-3-chlorophenylcarbamoyl)-piperazin-1-yl]-7-(3-fluoro-4-methyl-phenyl)-pyrido[3,2-d]pyrimidine (example 74) was obtained from 3-fluoro-4-methylphenyl boronic acid (yield 92%) as a white solid which was characterized by its mass spectrum as follows: MS (m/z): 477.1 ([M+H]⁺, 100),
4-[(N-3-chlorophenylcarbamoyl)-piperazin-1-yl]-7-(3-chloro-4-fluoro-phenyl)-pyrido[3,2-d]pyrimidine (example 75) was obtained from 3-chloro-4-fluoro-phenyl boronic acid (yield 86%) as a white solid which was characterized by its mass spectrum as follows: MS (m/z): 497.2 ([M+H]⁺, 100),
4-[(N-3-chlorophenylcarbamoyl)-piperazin-1-yl]-7-(3,4-methylenedioxy-phenyl)-pyrido[3,2-d]pyrimidine (example 76) was obtained from 3,4-methylenedioxyphenylboronic acid (yield 87%) as a white solid which was characterized by its mass spectrum as follows: MS (m/z): 489.2 ([M+H]⁺, 100),
4-[(N-3-chlorophenylcarbamoyl)-piperazin-1-yl]-7-(3-chloro-4-ethoxy-phenyl)-pyrido[3,2-d]pyrimidine (example 77) was obtained from 3-chloro-4-ethoxyphenylboronic acid (yield 81%) as a white solid which was characterized by its mass spectrum as follows: MS (m/z): 523.2 ([M+H]⁺, 100), and
4-[(N-3-chlorophenylcarbamoyl)-piperazin-1-yl]-7-(3-fluoro-4-ethoxy-phenyl)-pyrido[3,2-d]pyrimidine (example 78) was obtained from 3-fluoro-4-ethoxyphenyl boronic acid (yield 88%) as a white solid which was characterized by its mass spectrum as follows: MS (m/z): 507.2.2 ([M+H]⁺, 100).

### EXAMPLES 79 TO 84

### Synthesis of 4-[(N-3-chlorophenylcarbamoyl)-piperazin-1-yl]-6-aryl-pyrido[3,2-d]pyrimidines

The procedure of examples 71 to 78 was repeated, using 4-[(N-3-chloro-phenylcarbamoyl)-piperazin-1-yl]-6-chloro-pyrido[3,2-d]pyrimidine as the starting material, for preparing the following pure compounds:
4-[(N-3-chlorophenylcarbamoyl)-piperazin-1-yl]-6-(3-chloro-4-methoxyphenyl)-pyrido[3,2-d]pyrimidine (example 79) was obtained from 3-chloro-4-methoxy-phenyl boronic acid (yield 86%) as a white solid which was characterized by its mass spectrum as follows: MS (m/z): 509.1 ([M+H]⁺, 100),
4-[(N-3-chlorophenylcarbamoyl)-piperazin-1-yl]-6-(1,4-benzodioxan-6-yl)-pyrido[3,2-d]pyrimidine (example 80) was obtained from 1,4-benzodioxane-6-boronic acid (yield 93%) as a white solid which was characterized by its mass spectrum as follows: MS (m/z): 503.2 ([M+H]⁺, 100),
4-[(N-3-chlorophenylcarbamoyl)-piperazin-1-yl]-6-(3,4-dimethylphenyl-pyrido[3,2-d]pyrimidine (example 81) was obtained from 3,4-dimethylphenyl boronic acid (yield 80%) as a white solid which was characterized by its mass spectrum as follows: MS (m/z): 473.2 ([M+H]⁺, 100),
4-[(N-3-chlorophenylcarbamoyl)-piperazin-1-yl]-6-(3,4-methylenedioxy)phenyl-pyrido[3,2-d]pyrimidine (example 82) was obtained from 3,4-methylenedioxyphenyl boronic acid (yield 92%) as a white solid which was characterized by its mass spectrum as follows: MS (m/z): 489.2 ([M+H]⁺, 100),
4-[(N-3-chlorophenylcarbamoyl)-piperazin-1-yl]-6-(3-chloro-4-ethoxyphenyl-pyrido[3,2-d]pyrimidine (example 83) was obtained from 3-chloro-4-ethoxyphenylboronic acid (yield 92%) as a white solid which was characterized by its mass spectrum as follows: MS (m/z): 523.1 ([M+H]⁺, 100), and
4-[(N-3-chlorophenylcarbamoyl)-piperazin-1-yl]-6-(3,4-dichlorophenyl-pyrido[3,2-d]pyrimidine (example 84) was obtained from 3,4-dichlorophenyl boronic acid (yield 76%) as a white solid which was characterized by its mass spectrum as follows: MS (m/z): 515.1 ([M+H]⁺, 100).

### EXAMPLE 85

### Synthesis of 6-chloro-pyrido[3,2-d]pyrimidin-2(1H)-4(3H)-dione

Adding triphosgene (3.05 g, 10.14 mmole) to a solution of 6-chloro-2-carboxamido-3-amino-pyridine (3.48 g, 20.28 mmole) in dry dioxane (125 ml) under a N₂ atmosphere resulted in the immediate formation of a precipitate. The dark orange reaction mixture was stirred under reflux under a N₂ atmosphere for 30 minutes. Upon cooling, the solvent was removed under reduced pressure and the residue was purified by silica gel flash chromatography, the mobile phase being a CH₃OH/CH₂Cl₂ mixture (in a ratio gradually ranging from 5:95 to 15:95), resulting in the pure title compound as a white powder (2.96 g, yield 74%) which was characterized by its mass spectrum as follows: MS (m/z): 198 ([M+H]⁺, 100).

### EXAMPLE 86

### Synthesis of 6-(3,4-dimethoxy-phenyl)-pyrido[3,2-d]pyrimidin-2(1H)-4-(3H)-dione

A suspension of 6-chloro-pyrido[3,2-d]pyrimidin-2(1H)-4(3H)-dione (300 mg, 1.52 mmole), K₂CO₃ (840 mg, 6 mmole) and 3,4-dimethoxyphenylboronic acid (360 mg, 1.98 mmole) in 1,4-dioxane (22.5 ml) and water (8 ml) was purged with a nitrogen stream for 15 minutes. Tetrakis(triphenylphosphine)palladium(0) (90 mg, 76 mmole) was added and the mixture was heated to reflux for 24 hours. Upon cooling, the reaction mixture was filtered. The solid residue was recrystallized from hot acetic acid, then washed successively with acetic acid, ethyl acetate and diethyl ether, and finally dried, resulting in the pure title compound (297 mg, yield 65%) which was characterized by its mass spectrum as follows: MS (m/z): 300 ([M+H]⁺, 100).

### EXAMPLE 87

### Synthesis of 2,4-dichloro-6-(3,4-dimethoxyphenyl)-pyrido[3,2-d]pyrimidine

6-(3,4-dimethoxyphenyl)-pyrido[3,2-d]pyrimidin-2(1H)-4(3H)-dione (2.39 g, 7.97 mmole) was suspended in POCl₃ (54 ml) and triethylamine (3.1 ml, 21.8 mmole) was added. The dark brown mixture was stirred at reflux for 2.5 hours and allowed to cool down to room temperature. Most of POCl₃ was removed under reduced pressure and the rest was poured into ice/water and extracted with dichloromethane. The crude residue was purified by silica gel flash chromatography, the mobile phase being a n-hexane/EtOAc mixture, in a ratio gradually ranging from 1.5:1 to 1:1, to afford the pure title compound (1.69 g, yield 63%) which was characterized by its mass spectrum as follows: MS (m/z): 336 [(M+H)⁺, 100].

### EXAMPLE 88

### Synthesis of 2-morpholino-4-[(N-3-methyl-Phenylcarbamoyl-piperazin-1-yl]-6-(3,4-dimethoxyphenyl)-pyrido[3,2-d]pyrimidine

2-chloro-4-[(N-3-methyl-phenylcarbamoyl)-piperazin-1-yl]-6-(3,4-dimethoxyphenyl)-pyrido[3,2-d]pyrimidine (156 mg, 0.3 mmole) was suspended in 1,4-dioxane (10 ml) and morpholine (0.6 mmole) was added. The reaction mixture was heated at reflux for 4 hours, allowed to cool down to room temperature and partitioned between dichloromethane and a saturated aqueous sodium bicarbonate solution. The solid residue from the organic phase was purified by preparative thin layer chromatography on silica using a mixture of ethyl acetate and n-hexane (in a ratio of 1:4) as the mobile phase, to afford the pure title compound (21 mg, yield 12%) which was characterized by its mass spectrum as follows: MS (m/z): 570 ([M+H]⁺, 100).

### EXAMPLE 89

### Synthesis of 2-butoxy-4-[(N-3-methyl-phenylcarbamoyl)-piperazin-1-yl]-6-(3,4-dimethoxyphenyl)-pyrido[3,2-d]pyrimidine

28 mg (0.7 mmole) of 60% by weight NaH in mineral oil was suspended in dry tetrahydrofuran (5 ml) under a N₂ atmosphere, followed by the addition of n-butanol (0.6 mmole). Then, 2-chloro-[(N-3-methyl-phenylcarbamoyl)-piperazin-1-yl]-6-(3,4-dimethoxyphenyl)-pyrido[3,2-d]pyrimidine (149 mg, 0.29 mmole) was added. The mixture was heated at reflux under N₂ for 2.5 hours and then diluted with water. The crude product was extracted four times from the reaction mixture with ethyl acetate. The organic extracts were combined, dried over MgSO₄ and evaporated to dryness under reduced pressure. Preparative thin layer chromatography on silica using a n-hexane/ethyl acetate 1:4 mixture as eluent afforded the pure title compound (148 mg, yield 93%) which was characterized by its mass spectrum as follows: MS (m/z): 557 ([M+H]⁺, 100).

### EXAMPLE 90

### Synthesis of 2-methoxy-4-[(N-3-methyl-phenylcarbamoyl)-piperazin-1-yl]-6-(3,4-dimethoxy-phenyl)-pyrido[3,2-d]pyrimidine

24 mg (0.6 mmole) of 60% by weight NaH in mineral oil was suspended in dry tetrahydrofuran (3 ml) under a N₂ atmosphere followed by the addition of methanol (0.4 mmole). The mixture was stirred at room temperature for 15 minutes, and 2-chloro-4-[(N-3-methyl-phenylcarbamoyl)-piperazin-1-yl]-6-(3,4-dimethoxy phenyl)-pyrido[3,2-d]pyrimidine (104 mg, 0.2 mmole) was added. The solution was heated at reflux under N₂ for 1 hour and diluted with water. The crude product was extracted from the reaction mixture with ethyl acetate and the organic layer was washed with brine, dried over MgSO₄ and evaporated to dryness under reduced pressure. Preparative thin layer chromatography on silica, using a n-hexane/ethyl acetate mixture in a ratio of 1:5 as eluent, afforded the pure title compound (52 mg, yield 51%) which was characterized by its mass spectrum as follows: MS (m/z): 515 ([M+H]⁺, 100).

### EXAMPLE 91

### Synthesis of 2-(β-tolylamino)-4-[(N-3-methylphenylcarbamoyl)-piperazin-1-yl]-6-(3,4-dimethoxy-phenyl)-pyrido[3,2-d]pyrimidine

A white suspension of 2-chloro-4-[(N-3-methyl-phenylcarbamoyl)-piperazin-1-yl]-6-(3,4-dimethoxyphenyl)-pyrido[3,2-d]pyrimidine (104 mg, 0.2 mmole), K₂CO₃ (64 mg, 0.46 mmole), and p-toluidine (46 mg, 0.43 mmole) in a mixture of 1,4-dioxane/t-BuOH 5:1 (2 ml) was stirred at room temperature under nitrogen for 5 minutes. Thereafter, tetrakis(triphenylphosphine)palladium(0) (26 mg, 23 pmole) was added and the reaction mixture was heated at reflux under a N₂ atmosphere for 48 hours. Upon cooling, the mixture was diluted with water and extracted three times with ethyl acetate (brine added). The combined organic extracts were dried over Na₂SO₄, filtered and evaporated under reduced pressure. The crude residue was purified by column chromatography on silica using an ethyl acetate/n-hexane mixture as the mobile phase (in a ratio gradually ranging from 1:1 to 3:1), resulting in the pure title compound (30 mg, yield 25%) which was characterized by its mass spectrum as follows: MS (m/z): 590 ([M+H]⁺, 100).

### EXAMPLE 92

### Synthesis of 2-[(3-chloro-4-fluoro-anilino)-4-[(N-3-methyl-phenylcarbamoyl)-piperazin-1-yl]-6-(3,4-dimethoxyphenyl)-pyrido[3,2-d]pyrimidine

A suspension of 2-chloro-4-[(N-3-methyl-phenylcarbamoyl)-piperazin-1-yl]-6-(3,4-dimethoxyphenyl)-pyrido[3,2-d]pyrimidine (106 mg, 0.20 mmole), K₂CO₃ (62 mg, 0.45 mmole) and 3-chloro-4-fluoroaniline (60 mg 0.40 mmole) in a 1,4-dioxane/t-BuOH 5:1 mixture (2 ml) was purged with nitrogen for 15 minutes. Thereafter, tetrakis(triphenylphosphine)palladium(0) (28 mg, 24 μmol) was added and the reaction mixture was heated at reflux under a N₂ atmosphere for 20 hours. Upon cooling, the mixture was partitioned between ethyl acetate and brine. The organic phase was evaporated under reduced pressure and the crude residue was purified by flash chromatography on silica, using an ethyl acetate/n-hexane mixture as the mobile phase (in a ratio gradually ranging from 1:1 to 4:1), thus affording the pure title compound (60 mg, yield 47%) which was characterized by its mass spectrum as follows: MS (m/z): 628 ([M+H]⁺, 100).

### EXAMPLE 93

### Synthesis of 2,4-diamino-6-(4-hydroxy-3-methoxyphenyl)-pyrido[3,2-d]pyrimidine

A suspension of 2,4-diamino-6-chloropyrido[3,2-d]pyrimidine (378 mg, 1.93 mmole), K₂CO₃ (1075 mg, 7.78 mmole) and 2-methoxy-4-(4,4,5,5-tetramethyl-[1,3,2]dioxaborolan-2-yl)-phenol (599 mg, 2.32 mmole) in 1,4-dioxane (29 ml) and water (6 ml) was purged with a nitrogen stream for 30 minutes. Then, tetrakis(triphenylphosphine)palladium(0) (240 mg, 0.21 mmole) was added and purging with N₂ was continued for 15 minutes. The reaction mixture was then heated at reflux under a N₂ atmosphere for 2 hours. Upon cooling, the mixture was partitioned between CH₂Cl₂ and brine and the organic phase was dried over Na₂SO₄, filtered and evaporated under reduced pressure. Purification of the residue by silica gel flash chromatography with 10% methanol and 1% Et₃N in CH₂Cl₂ as mobile phase, afforded the pure title compound (375 mg, yield 69%) which was characterized by its mass spectrum as follows: MS (m/z): 284 ([M+H]⁺, 100).

### EXAMPLE 94

### Synthesis of 2,4-diamino-6-(3-chloro-4-methoxyphenyl)-pyrido[3,2-d]pyrimidine

A suspension of 2,4-diamino-6-chloropyrido[3,2-d]pyrimidine (464 mg, 2.37 mmole), K₂CO₃ (1332 mg, 9.64 mmole), 3-chloro-4-methoxyphenyl boronic acid (907 mg, 4.86 mmole) in 1,4-dioxane (35.5 ml) and water (7 ml) was purged with a stream of nitrogen for 15 minutes. Then, tetrakis(triphenylphosphine)palladium(0) (278 mg, 0.24 mmole) was added and the reaction mixture was heated at reflux under a N₂ atmosphere for 4 hours. Upon cooling, the mixture was partitioned between CH₂Cl₂ and a saturated aqueous sodium bicarbonate solution. The organic phase was dried over Na₂SO₄, filtered and evaporated under reduced pressure. The crude residue was purified by silica gel flash chromatography, using methanol and 1% Et₃N in CH₂Cl₂ as eluent, gradually increasing the methanol concentrations from 5% to 10%, to afford the pure title compound (277 mg, yield 39%) which was characterized by its mass spectrum as follows: MS (m/z): 302 ([M+H]⁺, 100).

### EXAMPLE 95

### Synthesis of 2-amino-6-(4-hydroxy-3-methoxy-phenyl)-pyrido[3,2-d]pyrimidin-4(3H)-one

A suspension of 2,4-diamino-6-(4-hydroxy-3-methoxy)-pyrido[3,2-d]pyrimidine (268 mg, 0.95 mmole) in 6 M aqueous HCl (7.6 ml) was refluxed for 26 hours. The cooled reaction mixture was stored at 4° C. for 16 hours. The yellow precipitate obtained was filtered off, washed with water until neutral pH value of the filtrate and dried to afford 243 mg (yield 90%) of the pure title compound which was characterized by its mass spectrum as follows: MS (m/z): 285 ([M+H]⁺, 100)

### EXAMPLE 96

### Synthesis of 2-amino-4-(N-morpholino)-6-(4-hydroxy-3-methoxy)-Pyrido[3,2-d]pyrimidine

A suspension of 2-amino-6-(4-hydroxy-3-methoxyphenyl)-pyrido[3,2-d]pyrimidin-4(3H)one (66 mg, 0.23 mmole), p-toluenesulphonic acid monohydrate (10 mg, 53 μmole), (NH₄)₂SO₄(11 mg, 83 μmole), 1,1,1,3,3,3-hexamethyldisilazane (1.15 mmole) and morpholine (1.83 mmole) in toluene (2 ml) was refluxed for 33 hours. The reaction mixture was allowed to cool down and partitioned between ethyl acetate and brine/saturated NaHCO₃ aqueous solution. The aqueous layer was extracted two times with ethyl acetate. The combined organic layers were dried over MgSO₄, filtered and evaporated under reduced pressure. The crude residue was purified by preparative thin layer chromatography on silica with 5% MeOH and 1% Et₃N in CH₂Cl₂ as mobile phase to afford the pure title compound (68 mg, yield 84%) which was characterized by its mass spectrum as follows: MS (m/z): 354 ([M+H]⁺, 100).

### EXAMPLE 97

### Synthesis of 2-amino-4-(N-morpholino)-6-(4-ethoxy-3-methoxyphenyl)-Pyrido[3,2-d]pyrimidine

A yellow suspension of 2-amino-4-(N-morpholino)-6-(4-hydroxy-3-methoxy-phenyl)-pyrido[3,2-d]pyrimidine (32 mg, 90 μmole), anhydrous potassium carbonate (30 mg, 0.22 mmole) and iodoethane (0.36 mmole) in acetone (2 ml) was refluxed under a nitrogen atmosphere. After 24 hours, second aliquots of K₂CO₃ and iodoethane were added and the reaction was continued for another 24 hours. Upon cooling, the reaction mixture was partitioned between EtOAc and a 5% aqueous sodium bicarbonate solution. The aqueous layer was extracted with ethyl acetate. The combined organic layers were dried over MgSO₄, filtered and evaporated under reduced pressure. Preparative thin layer chromatography of the crude residue on silica, using 5% methanol, 1% Et₃N in CH₂Cl₂ as mobile phase, afforded the pure title compound (26 mg, yield 76%) which was characterized by its mass spectrum as follows: MS (m/z): 382 ([M+H]⁺, 100).

### EXAMPLE 98

### Synthesis of 2-amino-4-(N-morpholino)-6-(4-cyclopentyloxy-3-methoxyphenyl)-pyrido[3,2-d]pyrimidine

A dark orange solution of 2-amino-4-(N-morpholino)-6-(4-hydroxy-3-methoxy-phenyl)-pyrido[3,2-d]pyrimidine (68 mg, 0.19 mmole), anhydrous potassium carbonate (53 mg, 0.38 mmole) and cyclopentyl iodide (0.75 mmole) in dimethylformamide (4 ml) was stirred at 60° C. After 24 hours, a second aliquot of cyclopentyl iodide was added and the reaction was continued for another 24 hours. Upon cooling, the reaction mixture was partitioned between ethyl acetate and brine/5% NaHCO₃ aqueous solution. The aqueous layer was extracted two times with ethyl acetate. The combined organic layers were dried over MgSO₄, filtered and evaporated under reduced pressure. Preparative thin layer chromatography of the crude residue on silica using 5% methanol in CH₂Cl₂ as mobile phase, afforded the pure title compound (6 mg, yield 7%) which was characterized by its mass spectrum as follows: MS (m/z): 422 ([M+H]⁺, 100).

### EXAMPLE 99

### Synthesis of 2-amino-4-(N-morpholino)-6-(4-isopropoxy-3-methoxy-phenyl)-pyrido[3,2-d]pyrimidine

To a yellow solution of 2-amino-4-(N-morpholino)-6-(3-methoxy-4-hydroxyphenyl)-pyrido[3,2-d]pyrimidine (107 mg, 0.30 mmole) in dry dimethyl-formamide (10 ml), was added 60% by weight NaH in mineral oil (0.93 mmole), resulting in an orange suspension. Then, 2-iodopropane (6.02 mmole) was added and the reaction mixture was stirred at room temperature for 40 minutes. The reaction mixture was partitioned between ethyl acetate and brine. The organic phase is dried over MgSO₄, filtered and evaporated under reduced pressure. Preparative thin layer chromatography of the crude residue on silica, using 5% methanol, 1% Et₃N in CH₂Cl₂ as mobile phase, afforded the title compound (83 mg, 70%) which was characterized by its mass spectrum as follows: MS (m/z): 396 ([M+H]⁺, 100).

### EXAMPLE 100

### Synthesis of 2-amino-4-(N-piperazin-1-yl)-6-(3-methoxy-4-hydroxy-phenyl)-pyrido[3,2-d]pyrimidine

A suspension of 2-amino-6-(4-hydroxy-3-methoxyphenyl)-pyrido[3,2-d]pyrimidin-4(3H)-one (227 mg, 0.80 mmole), p-toluenesulphonic acid monohydrate (88 μmole), (NH₄)₂SO₄ (0.12 mmole), 1,1,1,3,3,3-hexamethyldisilazane (3.98 mmole) and piperazine (11.72 mmole) in toluene (3 ml) was refluxed for 24 hours. Upon cooling, the reaction mixture was partitioned between ethyl acetate and 5% NaHCO₃ aqueous solution/brine. The aqueous layer was extracted 3 times with ethyl acetate. The combined organic layers were dried over MgSO₄, filtered and evaporated under reduced pressure. The crude residue was purified by preparative thin layer chromatography on silica using 15% methanol, 1% Et₃N in CH₂Cl₂ as mobile phase, affording the title compound (74 mg, yield 62%) which was characterized by its mass spectrum as follows: MS (m/z): 353 ([M+H]⁺, 100).

### EXAMPLE 101

### Synthesis of 2-amino-4-[(N-4-fluoro-phenyl-carbamoyl)-piperazin-1-yl]-6-(4-hydroxy-3-methoxy-phenyl)-pyrido[3,2-d]pyrimidine

A solution of 4-fluorophenyl isocyanate (0.39 mmole) in dimethylformamide (0.5 ml) was added to a yellow suspension of 2-amino-4-(N-piperazin-1-yl)-6-(4-hydroxy-3-methoxy)-pyrido[3,2-d]pyrimidine (0.31 mmole) in dimethylformamide (2 ml). The mixture was stirred at room temperature for 1 hour. The solvent was evaporated in vacuo. Preparative thin layer chromatography of the crude residue on silica using 5% methanol, 1% Et₃N in CH₂Cl₂ as mobile phase, afforded the pure title compound (100 mg, yield 66%) which was characterized by its mass spectrum as follows: MS (m/z): 490 ([M+H]⁺, 100).

### EXAMPLE 102

### Synthesis of 2-amino-4-[(N-4-fluoro-phenyl-carbamoyl-piperazin-1-yl)-6-(4-ethoxy-3-methoxyphenyl)-pyrido[3,2-d]pyrimidine

A suspension of 2-amino-4-[(N-4-fluoro-phenyl-carbamoyl)-piperazin-1-yl]-6-(4-hydroxy-3-methoxyphenyl)-pyrido[3,2-d]pyrimidine (0.13 mmole), anhydrous potassium carbonate (0.80 mmole) and iodoethane (1.23 mmole) in acetone (5 ml) was refluxed for 24 hours. Upon cooling, the reaction mixture was partitioned between ethyl acetate and brine. The aqueous layer was extracted with ethyl acetate. The combined organic layers were dried over MgSO₄, filtered and evaporated under reduced pressure. Preparative thin layer chromatography of the residue on silica using 5% methanol in CH₂Cl₂ as mobile phase, afforded the pure title compound (15 mg, yield 22%) which was characterized by its mass spectrum as follows: MS (m/z): 518 ([M+H]⁺, 100).

### EXAMPLE 103

### Synthesis of 2-amino-4-[(N-4-fluoro-phenyl-carbamoyl)-piperazin-1-yl]-6-(4-isopropoxy-3-methoxy-phenyl)-pyrido[3,2-d]pyrimidine

A suspension of 2-amino-4-[(N-4-fluoro-phenyl-carbamoyl)-piperazin-1-yl]-6-(4-hydroxy-3-methoxy-phenyl)-pyrido[3,2-d]pyrimidine (96 μmole), anhydrous potassium carbonate (0.22 mmole) and 2-iodopropane (0.96 mmole) in acetone (7 ml) was refluxed under a nitrogen atmosphere for 20 hours. Then, another aliquot of 2-iodopropane was added and the reaction was continued for another 24 hours. Upon cooling, the reaction mixture was partitioned between ethyl acetate and brine and the aqueous layer was extracted several times with ethyl acetate. The combined organic layers were dried over MgSO₄, filtered and evaporated under reduced pressure. Purification of the crude residue by silica gel flash chromatography, using 10% methanol in CH₂Cl₂ as mobile phase, afforded the pure title compound (20 mg, yield 39%) which was characterized by its mass spectrum as follows: MS (m/z): 532 ([M+H]⁺, 100).

### EXAMPLE 104

### Synthesis of 2-amino-4-[(N-3-methyl-phenyl-carbamoyl)-piperazin-1-yl]-6-(4-hydroxy-3-methoxyphenyl)-pyrido[3,2-d]pyrimidine

m-toluoyl isocyanate (0.55 mmole) was added to a suspension of 2-amino-4-(N-piperazin-1-yl)-6-(4-hydroxy-3-methoxyphenyl)-pyrido[3,2-d]pyrimidine (0.55 mmole) in dimethylformamide (7 ml). The mixture was stirred at room temperature for 20 minutes, and then partitioned between ethyl acetate and a 5% NaHCO₃ aqueous solution. The aqueous layer was extracted two times with ethyl acetate. The combined organic layers were dried over MgSO₄, filtered and evaporated under reduced pressure. Purification of the crude residue by preparative thin layer chromatography on silica using 5% methanol, 1% Et₃N in CH₂Cl₂ as eluent, afforded the pure title compound (123 mg, yield 46%) which was characterized by its mass spectrum as follows: MS (m/z): 486 ([M+H]⁺, 100).

### EXAMPLE 105

### Synthesis of 4-(4-methyl-phenyl-piperazin-1-yl)-6-(3,4-dimethoxy-phenyl)-pyrido[3,2-d]pyrimidine

To a suspension of 4-chloro-6-(3,4-dimethoxyphenyl)-pyrido[3,2-d]pyrimidine (0.597 mmole) in isopropanol (20 ml) was added 1-(4-methyl)phenyl-piperazine (1.2 mmole). The reaction mixture was heated at 80° C. for 2 hours, after which the suspension became a yellow solution. The solvent was evaporated in vacuo. The residue was redissolved in ethyl acetate and extracted with a NaOH solution (1 N). The combined organic layers were evaporated in vacuo and purified by silica gel column chromatography (the mobile phase being a mixture of methanol and dichloromethane in a ratio gradually ranging from 1:99 to 2:98), resulting in the title compound (191 mg, yield 73%) which was characterized by its mass spectrum as follows: MS (m/z): 442 ([M+H]⁺, 100).

### EXAMPLE 106

### Synthesis of 4-(4-fluorophenyl-piperazin-1-yl)-6-(3,4-dimethoxy-phenyl)-pyrido[3,2-d]pyrimidine

The procedure of example 105 was performed, but using 1-(4-fluoro)phenyl-piperazine as the starting material, thus resulting in the pure title compound which was characterized by its mass spectrum as follows: MS (m/z): 446 ([M+H]⁺, 100).

### EXAMPLE 107

### Synthesis of 4-(N-piperazin-1-yl)-6-(3,4-dimethoxyphenyl)-pyrido[3,2-d]-pyrimidine

To a suspension of 4-chloro-6-(3,4-dimethoxyphenyl)-pyrido[3,2-d]pyrimidine (1.47 mmole) in isopropanol (50 ml) was added piperazine (1.2 mmole). The reaction mixture was heated at 80° C. for 2 hours. Volatiles were evaporated in vacuo. The crude residue was purified by silica gel flash chromatography, the mobile phase being a methanol/dichloromethane mixture with an 0.5% aqueous NH₃ solution (in a ratio gradually ranging from 2:98 to 3:97), resulting in the pure title compound (351 mg, yield 68%) which was characterized by its mass spectrum as follows: MS (m/z): 352 ([M+H]⁺, 100).

### EXAMPLES 108 TO 112

### Synthesis of 4-(N-carbamoyl-piperazin-1-yl)-6-(3,4-dimethoxy-phenyl)-pyrido[3,2-d]pyrimidines

To a solution of 4-(N-piperazin-1-yl)-6-(3,4-dimethoxyphenyl)-pyrido[3,2-d]-pyrimidine (0.26 mmole) in dimethylformamide (20 ml) was added an appropriate isocyanate (0.39 mmole). The reaction mixture was stirred at room temperature for 2 hours. The solvents was evaporated in vacuo and the crude residue was purified by silica gel flash chromatography, the mobile phase being a mixture of methanol and dichloromethane in a ratio gradually ranging from 2:98 to 3:97, affording the pure title compounds in yields from 65 to 80% depending upon the relevant isocyanate. The following individual compounds were made according to this procedure:
4-[(N-3-chloro-4-fluorophenylcarbamoyl)-piperazin-1-yl]-6-(3,4-dimethoxy-phenyl)-pyrido[3,2-d]pyrimidine (example 108) was obtained from 3-chloro-4-fluorophenyl isocyanate and was characterized by its mass spectrum as follows: MS (m/z): 524 ([M+H]⁺, 100),
4-[(N-2-thienyl-carbamoyl)-piperazin-1-yl]-6-(3,4-dimethoxyphenyl-pyrido[3,2-d]pyrimidine (example 109) was obtained from 2-thienyl isocyanate and was characterized by its mass spectrum as follows: MS (m/z): 477 ([M+H]⁺, 100),
4-[(N-2,6-dichloro-pyridyl-carbamoyl)-piperazin-1-yl]-6-(3,4-dimethoxyphenyl-pyrido[3,2-d]pyrimidine (example 110) was obtained from 2,6-dichloro-4-isocyanato-pyridine and was characterized by its mass spectrum as follows: MS (m/z): 541 ([M+H]⁺, 100),
4-[(N-4-fluorophenylcarbamoyl)-piperazin-1-yl]-6-(3,4-dimethoxyphenyl)-pyrido[3,2-d]pyrimidine (example 111) was obtained from 4-fluorophenyl isocyanate and was characterized by its mass spectrum as follows: MS (m/z): 489 ([M+H]⁺, 100), and
4-[(N-3-chlorophenylcarbamoyl)-piperazin-1-yl]-6-(3,4-dimethoxyphenyl)-pyrido[3,2-d]pyrimidine (example 112) was obtained from 3-chlorophenyl isocyanate and was characterized by its mass spectrum as follows: MS (m/z): 506 ([M+H]⁺, 100).

### EXAMPLE 113

### Synthesis of 4-[(N-4-chlorophenoxy-acetyl)-piperazin-1-yl]-6-(3,4-dimethoxyphenyl)-pyrido[3,2-d]pyrimidine

To a solution of 4-(N-piperazin-1-yl)-6-(3,4-dimethoxyphenyl)-pyrido[3,2-d]-pyrimidine (0.18 mmole) in dimethylformamide (20 ml) was added triethylamine (0.26 mmole) and p-chloro-phenoxy acetyl chloride (0.23 mmole). The reaction mixture was stirred at room temperature for 3 hours, then quenched with water. The aqueous phase was extracted with dichloromethane. The combined organic layers were evaporated in vacuo. The residue was purified by silica gel flash chromatography, the mobile phase being a methanol/dichloromethane mixture in a ratio of 2:98, affording the pure title compound (66 mg, yield 71%) which was characterized by its mass spectrum as follows: MS (m/z): 521 ([M+H]⁺, 100).

### EXAMPLE 114

### Synthesis of 6-(3-methyl-4-methoxyphenyl)-pyrido[3,2-d]pyrimidin-4(3H)one

To a solution of 6-chloro-pyrido[3,2-d]pyrimidin-4(3H)one (1.94 mmole) in 1,4-dioxane (40 ml) and water (20 ml) was added 4-methoxy-3-methylphenyl boronic acid (2.33 mmole), potassium carbonate (4.85 mmole) and tetrakis(tri-phenylphosphine)palladium(0) (0.097 mmole). The reaction mixture was refluxed for two hours, cooled to room temperature and the solvents were evaporated in vacuo. The residue was adsorbed on silica and purified by silica gel column chromatography (the mobile phase being a methanol/dichloromethane mixture in a ratio of 3:97), affording the title compound as a pure white powder (398 mg, yield 77%) which was characterized by its mass spectrum as follows: MS (m/z): 268 ([M+H]⁺, 100).

### EXAMPLE 115

### Synthesis of 4-chloro-6-(3-methyl-4-methoxyphenyl)-pyrido[3,2-d]pyrimidine

To a suspension of 6-(3-methyl-4-methoxyphenyl)-pyrido[3,2-d]pyrimidin-4(3H)one (1.41 mmole) in toluene (80 ml) was added phosphorus oxychloride (4.23 mmole) and 2,6-lutidine (4.23 mmole). The reaction mixture was refluxed for 16 hours until a black solution was obtained. After evaporation to dryness, the residue was redissolved in ethyl acetate and extracted with a saturated sodium bicarbonate solution. The combined organic layers were evaporated in vacuo. The residue was purified by silica gel column chromatography (the mobile phase being a ethylacetate/hexane mixture in a ratio gradually ranging from 2:8 to 3:7), resulting in the pure title compound (300 mg, yield 74%) which was characterized by its mass spectrum as follows: MS (m/z): 287 ([M+H]⁺, 100).

### EXAMPLE 116

### Synthesis of 4-(piperazin-1-yl)-6-(3-methyl-4-methoxyphenyl)-pyrido[3,2-d]pyrimidine

To a suspension of 4-chloro-6-(3-methyl-4-methoxyphenyl)-pyrido[3,2-d]pyrimidine (0.99 mmole) in isopropanol (40 ml) was added piperazine (1.99 mmole). The reaction mixture was heated at 80° C. for 2 hours. The solvents were evaporated in vacuo. The crude residue was purified by silica gel flash chromatography (the mobile phase being a mixture of methanol and dichloromethane with an 0.5% aqueous NH₃ solution (in a ratio gradually ranging from 2:98 to 3:97), resulting in the pure title compound (259 mg, yield 78%) which was characterized by its mass spectrum as follows: MS (m/z): 336 ([M+H]⁺, 100).

### EXAMPLE 117

### Synthesis of 4-[(N-3-chloro-phenylcarbamoyl)-piperazin-1-yl]-6-(3-methyl-4-methoxyphenyl)-pyrido[3,2-d]pyrimidine

To a solution of 4-(N-piperazin-1-yl)-6-(3-methyl-4-methoxyphenyl)-pyrido[3,2-d]-pyrimidine (0.25 mmole) in DMF (30 ml) was added 3-chlorophenyl isocyanate (0.38 mmole). The reaction mixture was stirred at room temperature for 2 hours. The solvents were evaporated in vacuo and the crude residue was purified by silica gel flash chromatography, the mobile phase being a mixture of methanol and dichloro-methane in a ratio gradually ranging from 2:98 to 3:97, affording the pure title compound (81 mg, yield 66%) which was characterized by its mass spectrum as follows: MS (m/z): 490 ([M+H]⁺, 100).

### EXAMPLE 118

### Synthesis of 4-[(N-4-chloro-phenylcarbamoyl)-piperazin-1-yl]-6-(3-methyl-4-methoxyphenyl)-pyrido[3,2-d]pyrimidine

The procedure of example 117 was followed, but using 4-chlorophenyl isocyanate as the starting material. The pure title compound was isolated in a yield of 81% and was characterized by its mass spectrum as follows: MS (m/z): 490 ([M+H]⁺, 100).

### EXAMPLE 119

### Synthesis of 4-[(N-3-chloro-phenylcarbamoyl)-piperazin-1-yl]-6-(3-methoxy-4-hydroxyphenyl)-pyrido[3,2-d]pyrimidine

To a solution of 4-[(N-3-chloro-phenylcarbamoyl)-piperazin-1-yl]-6-chloro-pyrido[3,2-d]pyrimidine (0.51 mmole) in 1,4-dioxane (15 ml) and water (5 ml) was added 2-methoxy-4-(4,4,5,5-tetramethyl-[1,3,2]dioxaborolan-2-yl)-phenol (0.51 mmole), potassium carbonate (1.53 mmole) and tetrakis(triphenyl-phosphine)palladium(0) (0.02 mmole). The reaction mixture was refluxed for two hours, cooled down to room temperature and the solvents were evaporated in vacuo. The residue was purified by silica gel column chromatography (the mobile phase being an acetone/dichloromethane mixture in a ratio of 20:80), affording the title compound as a pure white powder (135 mg, yield 54%) which was characterized by its mass spectrum as follows: MS (m/z): 492 ([M+H]⁺, 100).

### EXAMPLE 120

### Synthesis of 4-[(N-3-chloro-phenylcarbamoyl)-piperazin-1-yl]-6-(3-methoxy-4-ethoxy-phenyl)-pyrido[3,2-d]pyrimidine

To a solution of 4-[(N-4-chloro-phenylcarbamoyl)-piperazin-1-yl]-6-(3-methoxy-4-hydroxyphenyl-pyrido[3,2-d]pyrimidine (0.19 mmole) in dry dimethyl-formamide (15 ml) was added potassium carbonate (0.19 mmole). This mixture was stirred at room temperature for 30 minutes under nitrogen and then, ethyl iodide (0.19 mmole) was added. The reaction mixture was stirred at room temperature for 16 hours. The solvent was evaporated in vacuo and the residue was purified by silica gel flash chromatography (the mobile phase being a methanol/dichloromethane mixture in a ratio of 2:98), affording the pure title compound as a white powder (67 mg, yield 68%) which was characterized by its mass spectrum as follows: MS (m/z): 520 ([M+H]⁺, 100).

### EXAMPLE 121

### Synthesis of 4-[(N-3-chloro-phenylcarbamoyl)-piperazin-1-yl]-6-(3-methoxy-4-isopropoxy-phenyl-pyrido[3,2-d]pyrimidine

The procedure of example 120 was followed, but using 2-iodopropane as the starting material. The pure title compound was isolated and characterized by its mass spectrum as follows: MS (m/z): 533 ([M+H]⁺, 100).

### EXAMPLE 122

### Synthesis of 4-[(N-3-chlorophenylacetyl)-piperazin-1-yl]-6-chloro pyrido[3,2-d]pyrimidine

A suspension of 3-chlorophenylacetic acid (2 mmole) in thionyl chloride (10 ml) was refluxed for 1 hour. The excess thionyl chloride was removed under reduced pressure to yield crude 3-chloro phenyl acetic acid chloride. This crude residue was redissolved in dichloromethane (10 ml) and this solution was added to a solution of 4-(piperazin-1-yl)-6-chloro-pyrido[3,2-d]pyrimidine (2 mmole) in dichloromethane (10 ml). The resulting mixture was stirred at room temperature for 1 hour. The solvents were removed by evaporation in vacuo. The crude residue was purified by silica gel column chromatography, the mobile phase being a MeOH/dichloromethane mixture in a ratio of 1:40, affording the pure title compound (yield 60%) as a yellowish solid which was characterized by its mass spectrum as follows: MS (m/z): 403.1 ([M+H]⁺, 100).

### EXAMPLE 123

### Synthesis of 4-morpholino-6-(3,4-dichlorophenyl)-pyrido[3,2-d]pyrimidine

The reaction of 4-morpholino-6-chloro-pyrido[3,2-d]pyrimidine and 3,4-dichlorophenylboronic acid afforded the pure title compound (yield 97%) as a yellowish solid which was characterized by its mass spectrum as follows: MS (m/z): 361.2 ([M+H]⁺, 100).

### EXAMPLE 124

### Synthesis of 4-morpholino-6-(4-chlorophenyl)-pyrido[3,2-d]pyrimidine

The reaction of 4-morpholino-6-chloro-pyrido[3,2-d]pyrimidine and 4-chloro-phenylboronic acid afforded the pure title compound (yield 92%) as a white solid solid which was characterized by its mass spectrum as follows: MS (m/z): 341.2 ([M+H]⁺, 100).

### EXAMPLE 125

### Synthesis of 4-[(N-3-chlorophenylacetyl)-piperazin-1-yl]-6-(3,4-dichlorophenyl)pyrido[3,2-d]pyrimidine

The reaction of 4-[(N-3-chlorophenylacetyl)piperazin-1-yl]-6-chloro-pyrido[3,2-d]pyrimidine and 3,4-dichlorophenyl boronic acid afforded the pure title compound (yield 86%) as a yellowish solid which was characterized by its mass spectrum as follows: MS (m/z): 512.2 ([M+H]⁺, 100).

### EXAMPLES 126 TO 132

### Synthesis of 2-amino-6-aryl-pyrido[3,2-d]pyrimidin-4(3H)-ones

To a degassed suspension of 2-amino-6-chloro-pyrido[3,2-d]pyrimidin-4(3H)-one (6 mmole), an appropriate aryl boronic acid (6.6 mmole) and potassium carbonate (30 mmole) in a mixture of dioxane (120 ml) and H₂O (30 ml), was added a catalytic amount of tetrakis(triphenylphosphine)palladium(0) (0.9 g). The mixture was refluxed for 24 hours and after cooling to room temperature, the reaction mixture was filtered. The filtrate was acidified with 5 N HCl to pH 4 and the resulting precipitate was filtered off, washed successively with H₂O, ethanol and diethylether, and further dried under vacuum to afford the desired compound in a yield between 65 and 85%, depending upon the relevant aryl boronic acid used. The following compounds were synthesized according to this procedure:
2-amino-6-(3-methoxy-4-methyl-phenyl)pyrido[3,2-d]pyrimidin-4(3H)-one (example 126) was obtained from 3-methoxy-4-methylphenyl boronic acid and was characterized by its mass spectrum as follows: MS (m/z): 317 ([M+H]⁺, 100),
2-amino-6-(3-chloro-4-ethoxy-phenyl)pyrido[3,2-d]pyrimidin-4(3H)-one (example 127) was obtained from 3-chloro-4-ethoxyphenyl boronic acid and was characterized by its mass spectrum as follows: MS (m/z): 317 ([M+H]⁺, 100),
2-amino-6-(3-ethoxy-4-fluoro-phenyl)pyrido[3,2-d]pyrimidin-4(3H)-one (example 128) was obtained from 3-ethoxy-4-fluorophenyl boronic acid and was characterized by its mass spectrum as follows: MS (m/z): 301 ([M+H]⁺, 100),
2-amino-6-(3-methyl-4-fluoro-phenyl)pyrido[3,2-d]pyrimidin-4(3H)-one (example 129) was obtained from 3-methyl-4-fluorophenyl boronic acid and was characterized by its mass spectrum as follows: MS (m/z): 271 ([M+H]⁺, 100),
2-amino-6-(3,4-dichloro-phenyl)pyrido[3,2-d]pyrimidin-4(3H)-one (example 130) was obtained from 3,4-dichlorophenyl boronic acid was characterized by its mass spectrum as follows: MS (m/z): 307 ([M+H]⁺, 100),
2-amino-6-(3,4-(methylenedioxy)phenyl)pyrido[3,2-d]pyrimidin-4(3H)-one (example 131) was obtained from 3,4-(methylenedioxy)phenyl boronic acid and was characterized by its mass spectrum as follows: MS (m/z): 283 ([M+H]⁺, 100), and
2-amino-6-(1,4-benzodioxane-phenyl)pyrido[3,2-d]-pyrimidin-4(3H)-one (example 132) was obtained from 1,4-benzodioxane-phenyl boronic acid and was characterized by its mass spectrum as follows: MS (m/z): 297 ([M+H]⁺, 100).

### EXAMPLES 133 TO 139

### Synthesis of 2-acetamido-6-aryl-pyrido[3,2-d]pyrimidin-4(3H)ones

A 2-amino-6-aryl-pyrido[3,2-d]pyrimidin-4(3H)-one (2.0 g) was suspended in acetic anhydride (180 ml) and acetic acid (20 ml) and the mixture was refluxed for 16 hours. The hot suspension was filtered and the filtrate was concentrated under reduced pressure until crystallization started. The precipitate was filtered off to give the pure title compound in a yield varying from 70 to 80%, depending upon the 6-aryl substituent being present in the starting material. The following compounds were synthesized according to this procedure:
2-acetamido-6-(3-methoxy-4-methyl-phenyl)pyrido[3,2-d]pyrimidin-4(3H)-one (example 133) was characterized by its mass spectrum as follows: MS (m/z): 325 ([M+H]⁺, 100),
2-acetamido-6-(3-chloro-4-ethoxy-phenyl)pyrido[3,2-d]pyrimidin-4(3H)-one (example 134) was characterized by its mass spectrum as follows: MS (m/z): 359 ([M+H]⁺, 100),
2-acetamido-6-(3-ethoxy-4-fluoro-phenyl)pyrido[3,2-d]pyrimidin-4(3-one (example 135) was characterized by its mass spectrum as follows: MS (m/z): 343 ([M+H]⁺, 100),
2-acetamido-6-(3-methyl-4-fluoro-phenyl)pyrido[3,2-a]pyrimidin-4(3H)-one (example 136) was characterized by its mass spectrum as follows: MS (m/z): 313 ([M+H]⁺, 100),
2-acetamido-6-(3,4-dichlorophenyl)pyrido[3,2-d]pyrimidin-4(3H)-one (example 137) was characterized by its mass spectrum as follows: MS (m/z): 349 ([M+H]⁺, 100),
2-acetamido-6-(3,4-(methylenedioxy)phenyl)pyrido[3,2-d]pyrimidin-4(3H)-one (example 138) was characterized by its mass spectrum as follows: MS (m/z): 325 ([M+H]⁺, 100), and
2-acetamido-6-(1,4-benzodioxane-phenyl)pyrido[3,2-d]pyrimidin-4(3H)-one (example 139) was characterized by its mass spectrum as follows: MS (m/z): 338 ([M+H]⁺, 100).

### EXAMPLES 140 TO 147

### Synthesis of 2-acetamido-4-(1,2,4-triazolyl)-6-aryl-pyrido[3,2-d]pyrimidines

A suspension of 1,2,4-triazole (120 mmole) and phosphorus oxychloride (36 mmole) in dry acetonitrile (150 ml) was added to a stirred suspension of a 2-acetamido-6-aryl-pyrido[3,2-d]pyrimidin-4(3H)-one (12 mmole) (obtained in examples 133 to 139) and triethylamine (36 mmole) in dry acetonitrile (150 ml). The mixture was stirred at room temperature under nitrogen for 70 hours and the yellow precipitate formed was filtered off, then successively washed with ethanol and ether, and further dried over P₂O₅ in a vacuum dessicator to afford the pure title compounds. Yields varied between 63% and 90%, depending upon the 6-aryl substituent being present. The following compounds were synthesized according to this procedure:
2-acetamido-4-(1,2,4-triazolyl)-6-(3-methyl-4-methoxyphenyl)pyrido-[3,2-d]pyrimidine (example 140) was characterized by its mass spectrum as follows: MS (m/z): 376 ([M+H]⁺, 100),
2-acetamido-4-(1,2,4-triazolyl)-6-(3-chloro-4-methoxy-phenyl)pyrido-[3,2-d]pyrimidine (example 141) was characterized by its mass spectrum as follows: MS (m/z): 396 ([M+H]⁺, 100),
2-acetamido-4-(1,2,4-triazolyl)-6-(3-chloro-4-ethoxy-phenyl)pyrido-[3,2-d]pyrimidine (example 142) was characterized by its mass spectrum as follows: MS (m/z): 411 ([M+H]⁺, 100),
2-acetamido-4-(1,2,4-triazolyl)-6-(3-fluoro-4-ethoxy-phenyl)pyrido-[3,2-d]pyrimidine (example 143) was characterized by its mass spectrum as follows: MS (m/z): 395 ([M+H]⁺, 100),
2-acetamido-4-(1,2,4-triazolyl)-6-(3-methyl-4-fluoro-phenyl)pyrido-[3,2-d]pyrimidine (example 144) was characterized by its mass spectrum as follows: MS (m/z): 365 ([M+H]⁺, 100),
2-acetamido-4-(1,2,4-triazolyl)-6-(3,4-dichloro-phenyl)pyrido-[3,2-d]pyrimidine (example 145) was characterized by its mass spectrum as follows: MS (m/z): 400 ([M+H]⁺, 100),
2-acetamido-4-(1,2,4-triazolyl)-6-(3,4-(methylenedioxy)phenyl)pyrido[3,2-d]pyrimidine (example 146) was characterized by its mass spectrum as follows: MS (m/z): 377 ([M+H]⁺, 100), and
2-acetamido-4-(1,2,4-triazolyl)-6-(1,4-benzodioxane-phenyl)pyrido[3,2-d]pyrimidin-4(3H)-one (example 147) was characterized by its mass spectrum as follows: MS (m/z): 381 ([M+H]⁺, 100).

### EXAMPLES 148 TO 155

### Synthesis of 2-acetamido-4-(N-piperazin-1-yl)-6-aryl-pyrido[3,2-d]pyrimidines

To a suspension of a 2-acetamido-4-(1,2,4-triazolyl)-6-aryl-pyrido[3,2-d]pyrimidine (1.25 mmole; obtained in examples 140 to 147) in dioxane (50 ml) was added piperazine (2.5 mmole). The reaction mixture was stirred for 16 hours at 50° C. The solvent was evaporated and the crude residue was purified by preparative thin layer chromatography on silica, using a methanol/dichloromethane mixture in a ratio of 20:80 as mobile phase, affording the pure title compounds in yields varying between 30 and 40%, depending upon the 6-aryl substituent being present. The following compounds were made according to this procedure:
2-acetamido-4-(N-piperazin-1-yl)-6-(3-methyl-4-methoxy-phenyl)pyrido-[3,2-d]pyrimidine (example 148) was characterized by its mass spectrum as follows: MS (m/z): 394 ([M+H]⁺, 100),
2-acetamido-4-(N-piperazin-1-yl)-6-(3-chloro-4-methoxy-phenyl)pyrido-[3,2-d]pyrimidine (example 149) was characterized by its mass spectrum as follows: MS (m/z): 414 ([M+H]⁺, 100),
2-acetamido-4-(N-piperazin-1-yl)-6-(3-chloro-4-ethoxy-phenyl)pyrido-[3,2-d]pyrimidine (example 150) was characterized by its mass spectrum as follows: MS (m/z): 428 ([M+H]⁺, 100),
2-acetamido-4-(N-piperazin-1-yl)-6-(3-fluoro-4-ethoxy-phenyl)pyrido-[3,2-d]pyrimidine (example 151) was characterized by its mass spectrum as follows: MS (m/z): 412 ([M+H]⁺, 100),
2-acetamido-4-(N-piperazin-1-yl)-6-(3-methyl-4-fluoro-phenyl)pyrido-[3,2-d]pyrimidine (example 152) was characterized by its mass spectrum as follows: MS (m/z): 382 ([M+H]⁺, 100),
2-acetamido-4-(N-piperazin-1-yl)-6-(3,4-dichloro-phenyl)pyrido-[3,2-d]pyrimidine (example 153) was characterized by its mass spectrum as follows: MS (m/z): 418 ([M+H]⁺, 100),
2-acetamido-4-(N-piperazin-1-yl)-6-(3,4-(methylenedioxy)phenyl)pyrido[3,2-d]pyrimidine (example 154) was characterized by its mass spectrum as follows, MS (m/z): 393 ([M+H]⁺, 100), and
2-acetamido-4-(N-piperazin-1-yl)-6-(1,4-benzodioxane-phenyl)pyrido[3,2-d]pyrimidine (example 155) was characterized by its mass spectrum as follows: MS (m/z): 407 ([M+H]⁺, 100).

### EXAMPLES 156 TO 162

### Synthesis of 2-acetamido-4-[(N-3-chloro-phenyl-carbamoyl)-piperazin-1-yl]-6-aryl-pyrido[3,2-d]pyrimidines and 2-amino-4-[(N-3-chloro-phenyl-carbamoyl)-piperazin-1-yl]-6-aryl-pyrido[3,2-d]pyrimidines

To a solution of a 2-acetamido-4-(piperazin-1-yl)-6-aryl-pyrido[3,2-d]pyrimidine (0.5 mmole) in dimethylformamide (5 ml) was added 3-chlorophenyl isocyanate (0.75 mmole). The reaction mixture was stirred for 16 hours at room temperature. The solvent was evaporated in vacuo, affording a crude 2-acetamido-4-[(N-3-chloro-phenyl-carbamoyl)-piperazin-1-yl]-6-aryl-pyrido[3,2-d]pyrimidine as an intermediate. This crude residue was dissolved in a mixture of CH₂Cl₂ (10 ml) and sodium ethoxide 0.2 N (10 ml). The suspension was stirred for 16 hours and neutralized with 5-6 N HCl in isopropyl alcohol, resulting in a crude 2-amino-4-[(N-3-chloro-phenyl-carbamoyl)-piperazin-1-yl]-6-aryl-pyrido[3,2-d]pyrimidine as the final product. This crude product was purified by preparative thin layer chromatography, the mobile phase consisting of CH₃OH/CH₂Cl₂ mixtures in a ratio of 10:90, yielding the pure title compounds, in yields varying from 20 to 40%, depending on the 6-aryl substituent being present. The following compounds were synthesized according to this procedure (each time through the corresponding intermediate having the 2-amino group protected in the form of acetamido):
2-amino-4-[(N-3-chloro-phenyl-carbamoyl)-piperazin-1-yl]-6-(3-methyl-4-methoxy-phenyl)pyrido-[3,2-d]pyrimidine (example 156) was characterized by its mass spectrum as follows: MS (m/z): 505 ([M+H]⁺, 100),
2-amino-4-[(N-3-chloro-phenyl-carbamoyl)piperazin-1-yl]-6-(3-chloro-4-methoxy-phenyl)pyrido-[3,2-d]pyrimidine (example 157) was characterized by its mass spectrum as follows: MS (m/z): 525 ([M+H]⁺, 100),
2-amino-4-[(N-3-chloro-phenyl-carbamoyl)piperazin-1-yl]-6-(3-chloro-4-ethoxy-phenyl)pyrido-[3,2-d]pyrimidine (example 158) was characterized by its mass spectrum as follows: MS (m/z): 538 ([M+H]⁺, 100),
2-amino-4-[(N-3-chloro-phenyl-carbamoyl)-piperazin-1-yl]-6-(3-fluoro-4-ethoxyphenyl)pyrido-[3,2-d]pyrimidine (example 159) was characterized by its mass spectrum as follows: MS (m/z): 523 ([M+H]⁺, 100),
2-amino-4-[N-3-chloro-phenyl-carbamoyl)-piperazin-1-yl]-6-(3,4-dichloro-phenyl)-pyrido-[3,2-d]pyrimidine (example 160) was characterized by its mass spectrum as follows: MS (m/z): 528 ([M+H]⁺, 100),
2-amino-4-[N-3-chloro-phenyl-carbamoyl)-piperazin-1-yl]-6-(3,4-(methylene-dioxy)phenyl)pyrido[3,2-d]pyrimidine (example 161) was characterized by its mass spectrum as follows: MS (m/z): 505 ([M+H]⁺, 100), and
2-amino-4-[(N-3-chloro-phenyl-carbamoyl)-piperazin-1-yl)-6-(1,4-benzo-dioxane-phenyl)pyrido[3,2-d]pyrimidine (example 162) was characterized by its mass spectrum as follows: MS (m/z): 519 ([M+H]⁺, 100).

### EXAMPLES 163 TO 165

### Synthesis of 2-amino-4-morpholino-6-aryl-pyrido[3,2-d]pyrimidines

To a suspension of a 2-acetamido-6-aryl-pyrido[3,2-d]pyrimidin-4(3H)-one (1 mmole) in toluene (10 ml) was added morpholine (4 mmole), p-toluene sulfonic acid (0.1 mmole), ammonium sulfate (0.1 mmole) and 1,1,1,3,3,3-hexamethyldisilazane (8 mmole). The reaction mixture was refluxed for 48 hours until a brown solution was formed. The solvent was evaporated in vacuo and the crude resulting residue was redissolved in dichloromethane and extracted successively with a saturated sodium bicarbonate aqueous solution and water. The combined organic layers were dried over sodium sulfate and evaporated in vacuo, resulting in a crude 2-amino-4-morpholino-6-aryl-pyrido[3,2-d]pyrimidine as a final product. This crude residue was purified by preparative thin layer chromatography on silica, using a methanol/dichloromethane mixture in a ratio of 10:90 as mobile phase, affording the pure final compounds in yields between 20 and 30%, depending on the 6-aryl substituent being present. The following final compounds were synthesized according to this procedure (each time through the corresponding intermediate having the 2-amino group protected in the form of acetamido):
2-amino-4-(morpholino)-6-(3-methyl-4-methoxyphenyl)pyrido[3,2-d]pyrimidine (example 163) was characterized by its mass spectrum as follows: MS (m/z): 352 ([M+H]⁺, 100),
2-amino-4-(morpholino)-6-(3-chloro-4-methoxyphenyl)pyrido[3,2-d]pyrimidine (example 164) was characterized by its mass spectrum as follows: MS (m/z) 372 ([M+H]⁺, 100), and
2-amino-4-(morpholino)-6-(1,4-benzodioxane-phenyl)pyrido[3,2-d]pyrimidine (example 165) was characterized by its mass spectrum as follows: MS (m/z): 366 ([M+H]⁺, 100).

### EXAMPLES 166 TO 168

### Synthesis of 2-amino-4-morpholino-6-aryl-pyrido[3,2-d]pyrimidines

To a suspension of a 2-acetamido-4-(1,2,4-triazolyl)-6-aryl-pyrido[3,2-d]pyrimidine (0.5 mmole) in dioxane (5 ml) was added morpholine (1 mmole). The reaction mixture was stirred for 16 hours at 50° C. The solvent was evaporated in vacuo yielding a crude 2-acetamido-4-morpholino-6-aryl-pyrido[3,2-d]pyrimidine as an intermediate product. This crude residue was dissolved in a mixture of CH₂Cl₂ (10 ml) and sodium ethoxide 0.2 N (10 ml). The suspension was stirred for 16 hours and neutralized with 5-6 N HCl in isopropyl alcohol, resulting in a crude 2-amino-4-morpholino-6-aryl-pyrido[3,2-d]pyrimidine as a final product. This crude product was purified by preparative thin layer chromatography, the mobile phase consisting of a CH₃OH/CH₂Cl₂ mixtures in a ratio of 10:90, affording the pure title compounds, in yields varying from 20 to 40% depending on the 6-aryl substituent being present. The following compounds were synthesized according to this procedure (each time through the corresponding intermediate having the 2-amino group protected in the form of acetamido):
2-amino-4-morpholino-6-(3-fluoro-4-ethoxy-phenyl)-pyrido[3,2-d]pyrimidine (example 166) was characterized by its mass spectrum as follows: MS (m/z): 370 ([M+H]⁺, 100),
2-amino-4-morpholino-6-(4-chlorophenyl)-pyrido[3,2-d]pyrimidine (example 167) was characterized by its mass spectrum as follows: MS (m/z): 342 ([M+H]⁺, 100), and
2-amino-4-morpholino-piperazin-1-yl]-6-(3,4-(methylenedioxy)phenyl)pyrido[3,2-d]pyrimidine (example 168) was characterized by its mass spectrum as follows: MS (m/z): 352 ([M+H]⁺, 100).

### EXAMPLES 169-173

### Synthesis of 2-amino-6-(aryl)-pyrido[3,2-d]pyrimidin-4(3H)-one analogues

### General Procedure

To a degassed suspension of 2-amino-6-chloro-pyrido[3,2-d]pyrimidin-4(3H)-one (1.96 g, 10 mmol), an appropriate phenyl boronic acid (11 mmol) and potassium carbonate (6.9 g, 50 mmol) in a mixture of dioxane (180 ml) and H₂O (50 ml), was added a catalytic amount of tetrakis(triphenylphosphine)palladium(0) (750 mg). The suspension was refluxed for 16 hours and finally became a solution. After cooling to room temperature, the reaction mixture was filtered. The filtrate was acidified with 5 N HCl to pH 4 and the resulting precipitate was filtered off. It was washed successively with H₂O, ethanol, diethylether and dried under vacuum to yield the desired product. The following compounds were synthesized according to this procedure:

### EXAMPLE 169

### 2-amino-6-(3-methyl-4-fluoro-phenyl)-pyrido[3,2-d]pyrimidin-4(3H)-one

Obtained from 3-methyl-4-fluoro-phenyl boronic acid in 70% yield.

MS (m/z): 271 ([M+H]⁺, 100)

### EXAMPLE 170

### 2-amino-6-(3,4-dichloro-phenyl)-pyrido[3,2-d]pyrimidin-4(3H)-one

Obtained from 3,4-dichlorophenyl boronic acid in 91% yield.

MS (m/z): 307, 309 ([M+H]⁺, 100)

### EXAMPLE 171

### 2-amino-6-(4-fluoro-phenyl)-pyrido[3,2-d]pyrimidin-4(3H)-one

Obtained from 4-fluoro-phenyl boronic acid in 78% yield.

MS (m/z): 257 ([M+H]⁺, 100)

### EXAMPLE 172

### 2-amino-6-(1,4-benzodioxane)-pyrido[3,2-d]pyrimidin-4(3H)-one

Obtained from 1,4-benzodioxane-6-boronic acid in 82% yield.

MS (m/z): 297 ([M+H]⁺, 100)

### EXAMPLE 173

### 2-amino-6-(3,4-methylenedioxyphenyl)-pyrido[3,2-d]pyrimidin-4(3H)-one

Obtained from 3,4-methylenendioxyphenyl boronic acid in 71% yield.

MS (m/z): 283 ([M+H]⁺, 100)

### EXAMPLE 174-178

### Synthesis of 2-acetamido-6-(aryl)-pyrido[3,2-d]pyrimidin-4(3H)-one analogues

2-Amino-6-aryl-pyrido[3,2-d]pyrimidin-4(3H)-one (10 mmol) was suspended in acetic anhydride (300 ml) and the mixture was refluxed for 2 hours till a clear solution was obtained. The solution was concentrated under reduced pressure until crystallization started. The precipitate was filtered off to give the pure title compound. The following compounds were synthesized according to this procedure:

### EXAMPLE 174

### 2-acetamido-6-(3-methyl-4-fluoro-phenyl)-pyrido[3,2-d]pyrimidin-4(3H)-one

Obtained from 2-amino-6-(3-methyl-4-fluoro-phenyl)-pyrido[3,2-d]pyrimidin-4(3H)-one in 90% yield.

MS (m/z): 313 ([M+H]⁺, 100)

### EXAMPLE 175

### 2-acetamido-6-(3,4-dichloro-phenyl)-pyrido[3,2-d]pyrimidin-4(3H)-one

Obtained from 2-amino-6-(3,4-dichloro-phenyl)-pyrido[3,2-d]pyrimidin-4(3H)-one in 90% yield.

MS (m/z): 349, 351 ([M+H]⁺, 100)

### EXAMPLE 176

### 2-acetamido-6-(4-fluoro-phenyl)-pyrido[3,2-d]pyrimidin-4(3H)-one

Obtained from 2-amino-6-(4-fluoro-phenyl)-pyrido[3,2-d]pyrimidin-4(3H)-one in 78% yield.

MS (m/z): 299 ([M+H]⁺, 100)

### EXAMPLE 177

### 2-acetamido-6-(1,4-benzodioxane)-pyrido[3,2-d]pyrimidin-4(3H)-one

Obtained from 2-amino-6-(1,4-benzodioxane)-pyrido[3,2-d]pyrimidin-4(3H)-one in 68% yield.

MS (m/z): 339 ([M+H]⁺, 100)

### EXAMPLE 178

### 2-acetamido-6-(3,4-methylenedioxyphenyl)-pyrido[3,2-d]pyrimidin-4(3H)-one

Obtained from 2-amino-6-(3,4-methylenedioxyphenyl)-pyrido[3,2-d]pyrimidin-4(3H)-one in 74% yield.

MS (m/z): 325 ([M+H]⁺, 100)

### EXAMPLE 179

### Synthesis of 2-amino-4-(morpholino)-6-(3-methyl-4-fluoro-phenyl)-pyrido[3,2-d]pyrimidine

To a suspension of 2-acetamido-6-(3-methyl-4-fluorophenyl)-pyrido[3,2-d]pyrimidin-4(3H-one (312 mg, 1 mmol) in toluene (10 ml) was added morpholine (4 mmol, 0.23 ml), p-toluene sulfonic acid (0.1 mmol, 19 mg), ammonium sulfate (13 mg, 0.1 mmol) and 1,1,1,3,3,3-hexamethyldisilazane (2 ml, 8 mmol). The reaction mixture was refluxed for 48 hours till a brown solution was formed. The solvents were evaporated in vacuo, yielding crude 2-acetamido-4-(morpholino)-6-(4-methyl-3-fluoro-phenyl)-pyrido[3,2-d]pyrimidine. The residue was redissolved in a mixture of dichloromethane and ethanol (in a ratio of 80/20, 10 ml). A sodium ethoxide solution (0.2 N solution) was added till pH 12 and the resulting mixture was stirred overnight at room temperature. The solvents were evaporated in vacuo. The crude residue was purified by preparative TLC on silica, using a methanol/dichloromethane mixture in a ratio of 10:90 as mobile phase, yielding pure 2-amino-4-(morpholino)-6-(3-methyl-4-fluoro-phenyl)-pyrido[3,2-d]pyrimidine (80 mg, 25%).

MS (m/z): 340 ([M+H]⁺, 100)

UV (MeOH, m): 211, 278, 361

### EXAMPLE 180-183

### Synthesis of 2-acetamido-4-(1,2,4-triazolyl)-6-aryl-pyrido[3,2-d]pyrimidine

### General Procedure

A suspension of 1,2,4-triazole (345 mg, 5 mmol) and phosphorus oxychloride (0.11 ml, 1.25 mmol) in dry acetonitrile (10 ml) was stirred under a nitrogen atmosphere for 15 minutes. This suspension was added to another suspension of 2-acetamido-6-aryl-pyrido[3,2-d]pyrimidin-4(3H)-one (1 mmol) and triethylamine (0.4 ml, 3 mmol) in dry acetonitrile (10 ml). The resulting mixture was stirred at 50° C. under nitrogen for 24 hours. The solvents were evaporated in vacuo. The crude residue was redissolved in dichloromethane and extracted with a diluted hydrochloric acid solution (HCl 0.01 N). The combined organic layers were evaporated yielding the title compounds, which were used for further reaction without any additional purification. The following compounds were made according to this procedure:

### EXAMPLE 180

### 2-acetamido-4-(1,2,4-triazolyl)-6-(3,4-dichloro-phenyl)pyrido[3,2-d]pyrimidine

Obtained from 2-acetamido-6-(3,4-dichloro-phenyl)-pyrido[3,2-d]pyrimidin-4(3H)-one in 80% yield.

MS (m/z): 400, 402 ([M+H]⁺, 100)

### EXAMPLE 181

### 2-acetamido-4-(1,2,4-triazolyl)-6-(4-fluoro-phenyl)-pyrido[3,2-d]pyrimidine

Obtained from 2-acetamido-6-(4-fluoro-phenyl)-pyrido[3,2-d]pyrimidin-4(3H)-one in 72% yield.

MS (m/z): 350 ([M+H]⁺, 100)

### EXAMPLE 182

### 2-acetamido-4-(1,2,4-triazolyl)-6-(1,4-benzodioxane)-pyrido[3,2-d]pyrimidine

Obtained from 2-acetamido-6-(1,4-benzodioxane)-pyrido[3,2-d]pyrimidin-4(3H)-one in 59% yield.

MS (m/z): 390 ([M+H]⁺, 100)

### EXAMPLE 183

### 2-acetamido-4-(1,2,4-triazolyl)-6-(3,4-methylenedioxyphenyl)-pyrido[3,2-d]pyrimidine

Obtained from 2-acetamido-6-(3,4-methylenedioxyphenyl)-pyrido[3,2-d]pyrimidin-4(3H)-one in 68% yield.

MS (m/z): 376 ([M+H]⁺, 100)

### EXAMPLE 184

### Synthesis of 2-amino-4-(morpholino)-6-(3,4-dichlorophenyl)-pyrido[3,2-d]pyrimidine

To a suspension of 2-acetamido-4-(1,2,4-triazolyl)-6-(3,4-dichlorophenyl)-pyrido[3,2-d]pyrimidine (400 mg, 1 mmol) in dioxane (10 ml) was added morpholine (174 mg, 2 mmol). The reaction mixture was stirred overnight at 50° C. The solvents were evaporated in vacuo yielding crude 2-acetamido-4-(morpholino)-6-(3,4-dichlorophenyl)-pyrido[3,2-d]pyrimidine. The residue was redissolved in a mixture of dichloromethane and ethanol (in a ratio of 80/20, 10 ml). A sodium ethoxide solution (0.2 N solution) was added till pH 12 and the resulting mixture was stirred overnight at room temperature. The solvents were evaporated in vacuo. The crude residue was purified by preparative TLC on silica, using a methanol/dichloromethane mixture in a ratio of 10:90 as mobile phase, yielding the pure title compound (220 mg, 60%).

MS (m/z): 376, 378 ([M+H]⁺, 100)

UV (MeOH, m): 282, 365

### EXAMPLE 185-188

### Synthesis of 2-acetamido-4-(N-piperazin-1-yl)-6-(aryl)-pyrido[3,2-d]pyrimidine

To a suspension of 2-acetamido-4-(1,2,4-triazolyl)-6-aryl-pyrido[3,2-d]pyrimidine (1 mmol) in dioxane (20 ml) was added piperazine (172 mg, 2 mmol). The reaction mixture was stirred overnight at 50° C. The solvents were evaporated in vacuo and the crude residue was purified by preparative TLC on silica, using a methanol/dichloromethane mixture in a ratio of 10:90 as mobile phase, yielding the pure title compounds.

The following compounds were prepared according to this procedure:

### EXAMPLE 185

### 2-acetamido-4-(N-piperazin-1-yl)-6-(4-fluorophenyl)-pyrido[3,2-d]pyrimidine

Obtained from 2-acetamido-4-(1,2,4-triazolyl)-6-(4-fluorophenyl)-pyrido[3,2-d]pyrimidine in 68% yield.

MS (m/z): 368 ([M+H]⁺, 100)

### EXAMPLE 186

### 2-acetamido-4-(N-piperazin-1-yl)-6-(1,4-benzodioxane)-pyrido[3,2-d]pyrimidine

Obtained from 2-acetamido-4-(1,2,4-triazolyl)-6-(1,4-benzodioxane)-pyrido[3,2-d]pyrimidine

MS (m/z): 407 ([M+H]⁺, 100)

### EXAMPLE 187

### 2-acetamido-4-(N-piperazin-1-yl)-6-(3,4-methylenedioxyphenyl)-pyrido[3,2-d]pyrimidine

Obtained from 2-acetamido-4-(1,2,4-triazolyl)-6-(3,4-methylenedioxyphenyl)-pyrido[3,2-d]pyrimidine

MS (m/z): 393 ([M+H]⁺, 100)

### EXAMPLE 188

### 2-acetamido-4-(N-piperazin-1-yl)-6-(3,4-dichloro-phenyl)-pyrido[3,2-d]pyrimidine

Obtained from 2-acetamido-4-(1,2,4-triazolyl)-6-(3,4-dichlorophenyl)-pyrido[3,2-d]pyrimidine

MS (m/z): 416, 418 ([M+H]⁺, 100)

### EXAMPLE 189

### Synthesis of 2-amino-4-[(N-4-chloro-benzylcarbamoyl)-piperazin-1-yl]-6-(4-fluorophenyl)-pyrido[3,2-d]pyrimidine

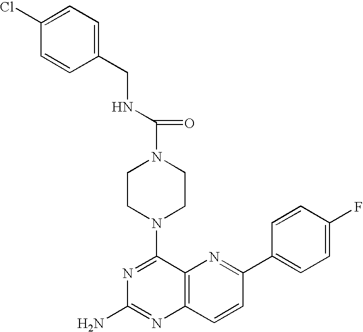

To a solution of 2-acetamido-4-(N-piperazin-1-yl)-6-(4-fluorophenyl)-pyrido[3,2-d]pyrimidine (367 mg, 1 mmol) in DMF (10 ml) was added 4-chloro-benzyl isocyanate (201 mg, 1.2 mmol). The solution was stirred overnight at room temperature. The solvents were evaporated in vacuo yielding crude 2-acetamido-4-[(N-4-chloro-benzyl-carbamoyl)-piperazin-1-yl]-6-(4-fluoro-phenyl)-pyrido[3,2-d]pyrimidine. The residue was redissolved in a mixture of dichloromethane and ethanol (in a ratio of 80/20, 10 ml). A sodium ethoxide solution (0.2 N solution) was added till pH 12 and the resulting mixture was stirred overnight at room temperature. The solvents were evaporated in vacuo. The crude residue was purified by preparative TLC on silica, using a methanol/dichloromethane mixture in a ratio of 10:90 as mobile phase, yielding the pure title compound (280 mg, 58%).

MS (m/z): 492, 494 ([M+H]⁺, 100)

UV (MeOH, m): 245, 350, 460, 560

### EXAMPLE 190

### Synthesis of 2-amino-4-[N-acetyl-piperazin-1-yl]-6-(3,4-methylenedioxyphenyl)-pyrido[3,2-d]pyrimidine

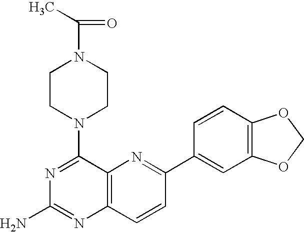

This compound was synthesized according to the procedure of example 184, using N-acetyl-piperazine and 2-acetamido-4-(1,2,4-triazolyl)-6-(3,4-methylenedioxy-phenyl)-pyrido[3,2-d]pyrimidine as starting materials.

MS (m/z): 393 ([M+H]⁺, 100)

### EXAMPLE 191

### Synthesis of 2-amino-4-[2-(piperazin-1-yl acetic acid N-(2-thiazolyl)-amide)]-6-3,4-methylenedioxyphenyl)-pyrido[3,2-d]pyrimidine

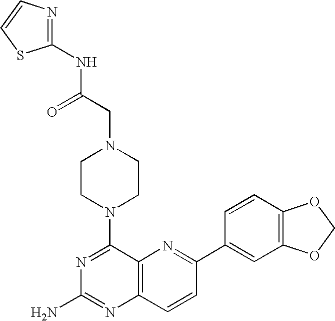

This compound was prepared according to the procedure of example 184, using 4-[2-(piperazin-1-yl acetic acid N-(2-thiazolyl)-amide) and 2-acetamido-4-(1,2,4-triazolyl)-6-(3,4-methylenedioxyphenyl)-pyrido[3,2-d]pyrimidine as starting materials.

MS (m/z): 491 ([M+H]⁺, 100)

### EXAMPLE 192

### Synthesis of 2-amino-4-[N-(2-furoyl)-piperazin-1-yl]-6-(3,4-methylenedioxyphenyl)-pyrido[3,2-d]pyrimidine

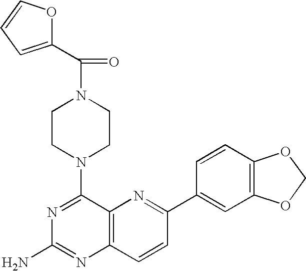

This compound was obtained using the procedure of example 184, using 2-furoyl-piperazine and 2-acetamido-4-(1,2,4-triazolyl)-6-(3,4-methylenedioxyphenyl)-pyrido[3,2-d]pyrimidine as starting materials.

MS (m/z): 445 ([M+H]⁺, 100)

### EXAMPLE 193

### Synthesis of 2-amino-4-[N-(4-chlorophenoxy-acetyl)-piperazin-1-yl]-6-(3,4-methylenedioxyphenyl)-pyrido[3,2-d]pyrimidine

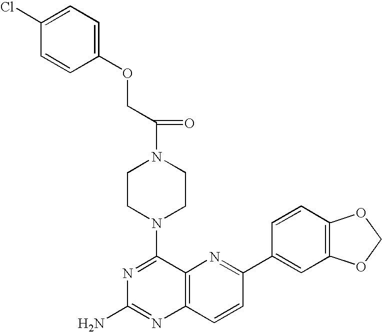

To a solution of 2-acetamido-4-(N-piperazin-1-yl)-6-(3,4-methylenedioxyphenyl)-pyrido[3,2-d]pyrimidine (60 mg, 0.16 mmol) in pyridine (5 ml) was added was added 4-chloro-phenoxy acetyl chloride (80 mg, 0.4 mmol). The solution was stirred overnight at 50° C. The solvents were evaporated in vacuo, thus yielding crude 2-acetamido-4-[N-(4-chlorophenoxy-acetyl)-piperazin-1-yl]-6-(3,4-methylenedioxy-phenyl)-pyrido[3,2-d]pyrimidine. The residue was redissolved in 5 ml of a dichloro-methane/ethanol mixture (in a volume ratio 80/20). A sodium ethoxide solution (0.2 N solution) was added till pH 12 and the resulting mixture was stirred overnight at room temperature. The solvents were evaporated in vacuo. The crude residue was purified by preparative TLC on silica, using a methanol/dichloromethane mixture in a volume ratio 10:90 as a mobile phase, yielding the pure title compound (48 mg, 47%).

MS (m/z): 519, 521 ([M+H]⁺, 100)

### EXAMPLE 194

### Synthesis of 2-amino-4-[N-(4-chlorophenoxy-acetyl)-piperazin-1-yl]-6-(3,4-dichlorophenyl)-pyrido[3,2-d]pyrimidine

This compound was obtained using the procedure described for the synthesis of example 193, using 2-acetamido-4-(N-piperazin-1-yl)-6-(3,4-dichlorophenyl)-pyrido[3,2-d]pyrimidine as starting material.

MS (m/z): 542, 544 ([M+H]⁺, 100)

### EXAMPLE 195

### Synthesis of 2-amino-4-[N-(4-chlorophenoxy-acetyl)-piperazin-1-yl]-6-(1,4-benzodioxane)-pyrido[3,2-d]pyrimidine

This compound was obtained using the procedure described for the synthesis of example 193, using 2-acetamido-4-(N-piperazin-1-yl)-6-(1,4-benzodioxane)-pyrido[3,2-d]pyrimidine as starting material. MS (m/z): 532, 534 ([M+H]⁺, 100).

### EXAMPLE 196

### Synthesis of 2-amino-4-[N-(3-methyl-phenyl-carbamoyl)-piperazin-1-yl]-6-(3,4-methylenedioxyphenyl)-pyrido[3,2-c]pyrimidine

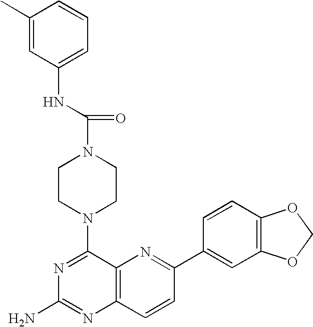

To a solution of 2-acetamido-4-(N-piperazin-1-yl)-6-(3,4-methylenedioxyphenyl)-pyrido[3,2-d]pyrimidine (60 mg, 0.16 mmol) in DMF (5 ml) was added m-tolyl isocyanate (31 μl, 0.24 mmol). The solution was stirred overnight at room temperature. The solvents were evaporated in vacuo yielding crude 2-acetamido-4-[N-(3-methyl-phenyl-carbamoyl)-piperazin-1-yl]-6-(3,4-methylenedioxyphenyl)-pyrido[3,2-d]pyrimidine. The residue was redissolved in a mixture of dichloromethane and ethanol (in a ratio of 80/20, 5 ml). A sodium ethoxide solution (0.2 N solution) was added till pH 12 and the resulting mixture was stirred overnight at room temperature. The solvents were evaporated in vacuo. The crude residue was purified by preparative TLC on silica, using a methanol/dichloromethane mixture in a ratio of 10:90 as mobile phase, yielding the pure title compound (32 mg, 43%).

MS (m/z): 484 ([M+H]⁺, 100).

### EXAMPLE 197

### Synthesis of 2-amino-4-[N-(3-methyl-phenyl-carbamoyl)-piperazin-1-yl]-6-(3,4-dichlorophenyl)-pyrido[3,2-d]pyrimidine

This compound was synthesized according to the procedure of example 196, using 2-acetamido-4-(N-piperazin-1-yl)-6-(3,4-dichlorophenyl)-pyrido[3,2-d]pyrimidine as the starting material. MS (m/z): 507, 509 ([M+H]⁺, 100).

### EXAMPLE 198

### Synthesis of 2-amino-4-[N-(3-methyl-phenyl-carbamoyl)-piperazin-1-yl]-6-(1,4-benzodioxane)-pyrido[3,2-d]pyrimidine

This compound was synthesized according to the procedure of example 196, using 2-acetamido-4-(N-piperazin-1-yl)-6-(1,4-benzodioxane)-pyrido[3,2-d]pyrimidine as a starting material. MS (m/z): 498 ([M+H]⁺, 100).

### EXAMPLE 199

### Synthesis of 2-amino-4-[N-acetyl-piperazin-1-yl]-6-(1,4-benzodioxane)-Pyrido[3,2-d]pyrimidine

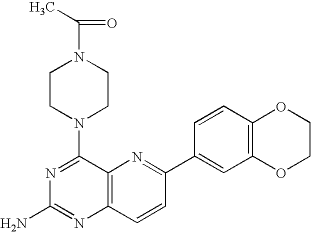

This compound was synthesized according to the procedure of example 184, using N-acetyl-piperazine and 2-acetamido-4-(1,2,4-triazolyl)-6-(1,4-benzodioxane)-pyrido[3,2-d]pyrimidine as starting materials. MS (m/z): 407 ([M+H]⁺, 100).

### EXAMPLE 200

### Synthesis of 2-amino-4-[N-acetyl-piperazin-1-yl]-6-(3,4-dichloro-Phenyl)-pyrido[3,2-d]pyrimidine

This compound was synthesized according to the procedure of example 184, using N-acetyl-piperazine and 2-acetamido-4-(1,2,4-triazolyl)-6-(3,4-dichlorophenyl)-pyrido[3,2-d]pyrimidine as starting materials. MS (m/z): 416, 418 ([M+H]⁺, 100).

### EXAMPLE 201

### Synthesis of 2-amino-4-[2-(piperazin-1-yl acetic acid N-(2-thiazolyl)-amide]-6-(1,4-benzodioxane)-pyrido[3,2-d]pyrimidine

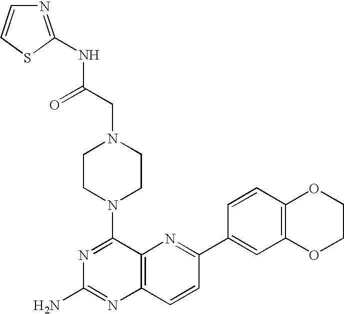

This compound was prepared according to the procedure of example 184, using 2-(piperazin-1-yi acetic acid)-N-(2-thiazolyl)-amide and 2-acetamido-4-(1,2,4-triazolyl)-6-(3,4-methylenedioxyphenyl)-pyrido[3,2-d]pyrimidine as starting materials.

MS (m/z): 505 ([M+H]⁺, 100)

### EXAMPLE 202

### Synthesis of 2-amino-4-[2-(piperazin-1-yl acetic acid N-(2-thiazolyl)-amide]-6-(3,4-dichlorophenyl)-pyrido[3,2-d]pyrimidine

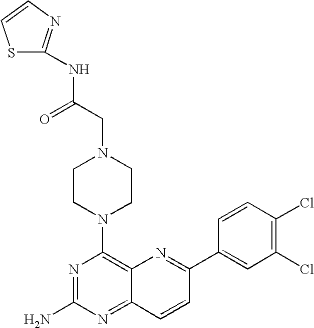

This compound was prepared according to the procedure of example 184, using 2-(piperazin-1-yl acetic acid)-N-(2-thiazolyl)-amide and 2-acetamido-4-(1,2,4-triazolyl)-6-(3,4-dichlorophenyl)-pyrido[3,2-d]pyrimidine as starting materials. MS (m/z): 514, 516 ([M+H]⁺, 100).

### EXAMPLE 203

### Synthesis of 2-amino-4-[N-(2-furoyl)-piperazin-1-yl]-6-(1,4-benzo-dioxane)-pyrido[3,2-o]pyrimidine

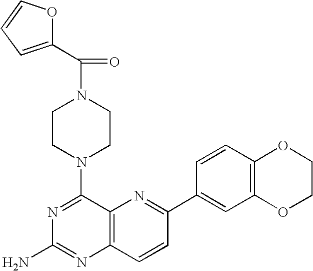

This compound was obtained using the procedure of example 184, using 2-furoyl-piperazine and 2-acetamido-4-(1,2,4-triazolyl)-6-(1,4-benzodioxane)-pyrido[3,2-d]pyrimidine as starting materials. MS (m/z): 459 ([M+H]⁺, 100).

### EXAMPLE 204

### Synthesis of 2-amino-4-[N-(4-fluoro-phenyl)-piperazin-1-yl]-6-(4-fluorophenyl)-pyrido[3,2-d]pyrimidine

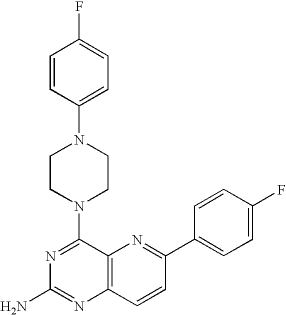

To a solution of 2-acetamido-4-(1,2,4-triazolyl)-6-(4-fluorophenyl)-pyrido[3,2-d]pyrimidine (367 mg, 1 mmol) in dioxane (10 ml) was added 1-(4-fluorophenyl)piperazine (360 mg, 2 mmol). The solution was stirred for 16 hours at 60° C. The solvents were evaporated in vacuo, yielding crude 2-acetamido-4-[N-(4-fluoro-phenyl)-piperazin-1-yl]-6-(4-fluoro-phenyl)-pyrido[3,2-d]pyrimidine. The residue was redissolved in 10 ml of a dichloromethane/ethanol mixture (in a volume ratio 80/20). A sodium ethoxide solution (0.2 N solution) was added till pH 12 and the resulting mixture was stirred for 16 hours at room temperature. The solvents were evaporated in vacuo. The crude residue was purified by preparative TLC on silica, using a methanol/dichloromethane mixture (volume ratio 10:90) as a mobile phase, yielding the pure title compound (280 mg, 69%) which was characterised as follows:

MS (m/z): 419 ([M+H]⁺, 100); and

UV (MeOH, m): 250, 345, 560.

### EXAMPLE 205

### Synthesis of 2-amino-4-[N-(phenoxy-ethyl)-piperazin-1-yl)]-6-(4-fluorophenyl)-pyrido[3,2-d]pyrimidine

To a suspension of 2-acetamido-4-(1,2,4-triazolyl)-6-(4-fluorophenyl)-pyrido[3,2-d]pyrimidine (367 mg, 1 mmol) in dioxane (10 ml) was added 1-(2-phenoxy-ethyl)-piperazine (412 mg, 2 mmol). The solution was stirred overnight at 60° C. The solvents were evaporated in vacuo yielding crude 2-acetamido-4-[N-(phenoxy-ethyl-piperazin-1-yl)]-6-(4-fluoro-phenyl)-pyrido[3,2-d]pyrimidine. The residue was redissolved in a mixture of dichloromethane and ethanol (in a ratio of 80/20, 10 ml). A sodium ethoxide solution (0.2 N solution) was added till pH 12 and the resulting mixture was stirred overnight at room temperature. The solvents were evaporated in vacuo. The crude residue was purified by preparative TLC on silica, using a methanol/dichloromethane mixture in a ratio of 10:90 as mobile phase, yielding the pure title compound (200 mg, 45%).

MS (m/z): 445 ([M+H]⁺, 100)

UV (MeOH, m): 250, 345, 495, 580

### EXAMPLE 206

### Synthesis of 2-amino-4-(anilino)-6-(4-fluorophenyl)-pyrido[3,2-d]pyrimidine

To a suspension of 2-acetamido-4-(1,2,4-triazolyl)-6-(4-fluorophenyl)-pyrido[3,2-d]pyrimidine (367 mg, 1 mmol) in dioxane (20 ml) was added aniline (186 mg, 2 mmol). The solution was stirred overnight at 60° C. The solvents were evaporated in vacuo yielding crude 2-acetamido-4-anilino-6-(4-fluoro-phenyl)-pyrido[3,2-d]pyrimidine. The residue was redissolved in a mixture of dichloromethane and ethanol (in a ratio of 80/20, 10 ml). A sodium ethoxide solution (0.2 N solution) was added till pH 12 and the resulting mixture was stirred overnight at room temperature. The solvents were evaporated in vacuo. The crude residue was purified by preparative TLC on silica, using a methanol/dichloromethane mixture in a ratio of 10:90 as mobile phase, yielding the pure title compound (160 mg, 50%).

MS (m/z): 332 ([M+H]⁺, 100)

UV (MeOH, m): 250, 350, 565

### EXAMPLE 207

### Synthesis of 2-amino-4-[(N-4-chloro-phenoxy-acetyl)-piperazin-1-yl]-6-(4-fluorophenyl)-pyrido[3,2-d]pyrimidine

To a solution of 2-acetamido-4-(N-piperazin-1-yl)-6-(4-fluorophenyl)-pyrido[3,2-d]pyrimidine (367 mg, 1 mmol) in pyridine (10 ml) was added 4-chloro-phenoxy acetyl chloride (410 mg, 2 mmol). The solution was stirred overnight at 50° C. The solvents were evaporated in vacuo yielding crude 2-acetamido-4-[(N-4-chloro-phenoxy-acetyl)-piperazin-1-yl]-6-(4-fluoro-phenyl)-pyrido[3,2-d]pyrimidine. The residue was redissolved in a mixture of dichloromethane and ethanol (in a ratio of 80/20, 10 ml). A sodium ethoxide solution (0.2 N solution) was added till pH 12 and the resulting mixture was stirred overnight at room temperature. The solvents were evaporated in vacuo. The crude residue was purified by preparative TLC on silica, using a methanol/dichloromethane mixture in a ratio of 10:90 as mobile phase, yielding the pure title compound (250 mg, 50%).

MS (m/z): 493, 495 ([M+H]⁺, 100)

UV (CH₃OH, m): 245, 345, 465, 560

### EXAMPLE 208

### Synthesis of 2-acetamido-6-chloro-pyrido[3,2-d]pyrimidin-4(3H)-one

A suspension of 2-amino-6-chloro-pyrido[3,2-d]pyrimidin-4(3H)-one (1.96 g, 10 mmol) in acetic anhydride (200 ml) was refluxed for 2 hours till a clear solution was obtained. The solvents were evaporated in vacuo till crystallization started. The precipitate was filtered off and dried under vacuum yielding the pure title compound (2 g, 80%).

MS (m/z): 239, 241 ([M+H]⁺, 100)

### EXAMPLE 209

### Synthesis of 2-amino-4-morpholino-6-chloro-pyrido[3,2-d]pyrimidine

To a suspension of 2-acetamido-6-chloro-pyrido[3,2-d]pyrimidin-4(3H)-one (2.38 g, 10 mmol) in dioxane (100 ml) was added diisopropylethylamine (5.3 ml, 30 mmol). The mixture was stirred for 10 minutes at 80° C., after which phosphorus oxychloride (1.4 ml, 15 mmol) was added. This reaction mixture was stirred for 90 minutes at 80° C. The solvents were evaporated in vacuo. The residue, was redissolved in dichloromethane and extracted with water. The combined organic layers were evaporated till a volume of 50 ml. Then, morpholine (870 mg, 10 mmol) was added and the reaction was stirred overnight at room temperature. The solvents were evaporated in vacuo. The residue was redissolved in a mixture of dichloromethane and ethanol (80/20, 100 ml). A sodium ethoxide solution (0.2 N solution) was added till pH=11. The mixture was stirred overnight at room temperature. The solvents were evaporated in vacuo. The residue was redissolved in dichloromethane and washed with water. The combined organic layers were combined and evaporated in vacuo, yielding the title compound (1 g, 40%).

MS (m/z): 266, 268 ([M+H]⁺, 100)

### EXAMPLE 210

### Synthesis of 2-amino-4-morpholino-6-(2-bromo-phenyl)-pyrido[3,2-d]pyrimidine

A solution of 2-amino-4-morpholino-6-chloro-pyrido[3,2-d]pyrimidine (265 mg, 1 mmol), potassium carbonate (690 mg, 5 mmol), tetrakis(triphenylphosphine)palladium(0) (100 mg) in dioxane (10 ml) and water (3 ml) was refluxed. To this refluxing solution was added dropwise (with a speed of 0.25 ml/min) a solution of 2-bromo-phenyl boronic acid (220 mg, 1.1 mmol) in dioxane (2 ml). Once the addition was complete, the reaction mixture was refluxed for another 2 hours. The reaction mixture was cooled down and the solvents were evaporated in vacuo. The residue was redissolved in dichloromethane and extracted with water. The combined organic layers were dried over Na₂SO₄ and the crude residue was purified by preparative TLC on silica, using a methanol/dichloromethane mixture in a ratio of 10:90 as mobile phase, yielding the pure title compound (100 mg, 30%).

MS (m/z): 386, 388 ([M+H]⁺, 100)

### EXAMPLE 211

### Synthesis of 4-[N-(3-chloro-phenylcarbamoyl)-piperazin-1-yl]-6-(3-methoxy-4-cyclopropylmethoxy-phenyl)-pyrido[3,2-d]pyrimidine

The procedure of example 120 was followed, but using cyclopropylmethyl bromide as a starting material. The pure title compound was isolated and characterized by its mass spectrum as follows: MS (m/z): 560, 562 ([M+H]⁺, 100).

### EXAMPLE 212

### Synthesis of 4-[N-(3-chloro-phenylcarbamoyl)-piperazin-1-yl]-6-(3-hydroxy-4-methoxy-phenyl)-pyrido[3,2-d]pyrimidine

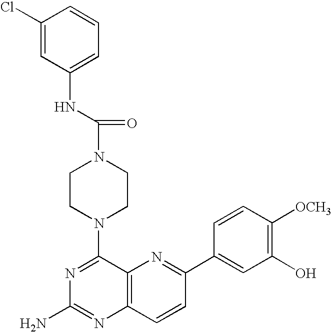

To a solution of 4-[N-(3-chloro-phenylcarbamoyl)-piperazin-1-yl]-6-chloro-pyrido[3,2-d]pyrimidine (650 mg, 1.61 mmol) in 1,4-dioxane (40 ml) and water (13 ml) was added 2-methoxy-5-(4,4,5,5-tetramethyl-1,3,2-dioxaborolan-2-yl)phenyl acetate (470 mg, 1.61 mmol), potassium carbonate (667 mg, 4.83 mmol) and tetrakis(triphenylphosphine)palladium(0) (93 mg, 0.0805 mmol). The reaction mixture was refluxed for 3 hours, then cooled down to room temperature and the solvents were evaporated in vacuo. The residue was purified by silica gel column chromatography, the mobile phase being an acetone/dichloromethane mixture (in a ratio ranging from 20:80 to 30:70), yielding the title compound as a pure white powder (513 mg, 63%). MS (m/z): 506, 508 ([M+H]⁺, 100).

### EXAMPLE 213-215

### Synthesis of 4-[(N-3-chloro-phenylcarbamoyl)-piperazin-1-yl]-6-(3-alkoxy-4-methoxy-phenyl)-pyrido[3,2-d]pyrimidine analogues

To a solution of 4-[N-(3-chloro-phenylcarbamoyl)-piperazin-1-yl]-6-(3-hydroxy-4-methoxy-phenyl)-pyrido[3,2-d]pyrimidine (100 mg, 0.20 mmol) in dry DMF (10 ml) was added potassium carbonate (42 mg, 0.3 mmol). This mixture was stirred at room temperature for 30 minutes under nitrogen and then, the appropriate alkyl halide (0.3 mmol) was added. After stirring for 5 hours, there was still starting material left and therefore an additional amount of the alkyl halide (0.3 mmol) and potassium carbonate (0.3 mmol) was added. The reaction mixture was further stirred at room temperature overnight. The solvents were evaporated in vacuo and purified by silica gel flash chromatography, the mobile phase being a mixture of methanol/dichloromethane (in a ratio ranging from 2:98 to 3:97), yielding the title compound as white powders, in yields varying from 60% to 70%, depending on the alkyl halide used.

The following compounds were synthesized according to this procedure:

### EXAMPLE 213

### 4-[N-(3-chloro-phenylcarbamoyl)-piperazin-1-yl]-6-(3-ethoxy-4-methoxy-phenyl)-pyrido[3,2-d]pyrimidine

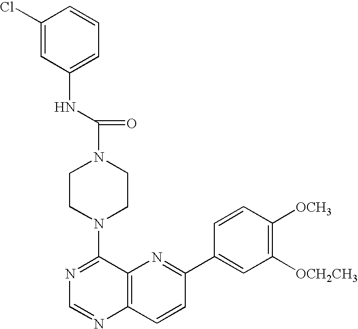

This compound was obtained from ethyl iodide as starting material.

MS (m/z): 534, 536 ([M+H]⁺, 100)

### EXAMPLE 214

### 4-[N-(3-chloro-phenylcarbamoyl)-piperazin-1-yl]-6-(3-isopropoxy-4-methoxy-phenyl)-pyrido[3,2-d]pyrimidine

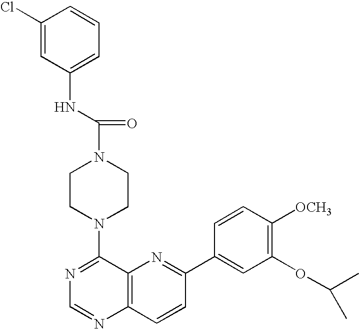

This compound was obtained from isopropyl iodide as starting material. MS (m/z) 548, 550 ([M+H]⁺, 100).

### EXAMPLE 215

### Synthesis of 4-[N-(3-chloro-phenylcarbamoyl)-piperazin-1-yl]-6-(3-cyclopropylmethoxy-4-methoxy-phenyl)-pyrido[3,2-d]pyrimidine

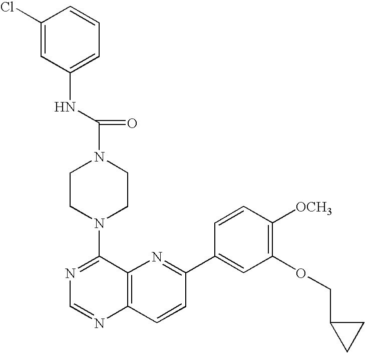

This compound was obtained from cyclopropylmethyl bromide as starting material.

MS (m/z): 560, 562 ([M+H]⁺, 100).

### EXAMPLE 216a

### Synthesis of 2-acetamido-4,6-dichloro-pyrido[3,2-d]pyrimidine

To a suspension of 2-acetamido-6-chloro-pyrido[3,2-d]pyrimidin-4(3H)-one (360 mg, 1.51 mmol) in dioxane (30 ml) was added diisopropylethylamine (788 μl, 4.53 mmol) and POCl₃ (422 μl, 4.53 mmol). The reaction was heated at 100° C. overnight till a black solution was obtained. The solvents were evaporated in vacuo. The crude residue was redissolved in dichloromethane and was extracted three times with ice-cold water. The combined organic layers were evaporated in vacuo and used for further reactions without any additional purification. MS (m/z): 257, 259 ([M+H]⁺, 100).

### EXAMPLE 216B & 216 C

### Synthesis of 2-acetamido-4-[(S)-3-(Boc-amino)pyrrolidine]-6-chloro-pyrido[3,2-d]pyrimidine

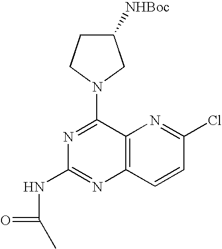

To a solution of 2-acetamido-4,6-dichloro-pyrido[3,2-d]pyrimidine (the crude residue obtained in the previous example 216a) in dioxane (20 ml) was added (S)-3-(Boc-amino)pyrrolidine (563 mg, 3.02 mmol). The reaction mixture was stirred at room temperature for 2 hours. The reaction was diluted with water and extracted with dichloromethane. The combined organic layers were evaporated in vacuo. The crude residue was purified by silica gel flash chromatography, the mobile phase being a MeOH/CH₂Cl₂ mixture in a ratio of 4:96, yielding two pure compounds, i.e.:
2-acetamido-4-[(S)-3-(Boc-amino)pyrrolidine]-6-chloro-pyrido[3,2-d]pyrimidine (216 b) (210 mg); MS (m/z): 257, 259 ([M+H]⁺, 100); and
2-amino-4-[(S)-3-(Boc-amino)pyrrolidine]-6-chloro-pyrido[3,2-d]pyrimidine (216 c) (43 mg); MS (m/z): 257, 259 ([M+H]⁺, 100).

### EXAMPLE 217

### Synthesis of 2-amino-4-[(S)-3-(Boc-amino)pyrrolidine]-6-chloro-pyrido[3,2-d]pyrimidine

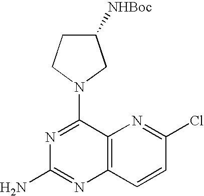

To a solution of 2-acetamido-4-[(S)-3-(Boc-amino)pyrrolidine]-6-chloro-pyrido[3,2-d]pyrimidine in methanol (10 ml) was added a solution of potassium carbonate (360 mg) in water (5 ml). The reaction was heated at 80° C. for 2 hours. The reaction was cooled down, diluted with water and extracted with dichloromethane. The combined organic layers were evaporated in vacuo and the crude residue was purified by flash chromatography on silica, the mobile phase being a mixture of acetone/CH₂Cl₂ (in a ratio of 40:60), followed by a mixture of CH₃OH/CH₂Cl₂ in a ratio of 4:96, yielding the title compound as a pure white solid (133 mg, 71%). MS (m/z): 365, 367 ([M+H]⁺, 100).

### EXAMPLE 218

### Synthesis of 2-amino-4-[(S)-3-(Boc-amino)pyrrolidine]-6-(3,4-dimethoxyphenyl)-pyrido[3,2-d]pyrimidine

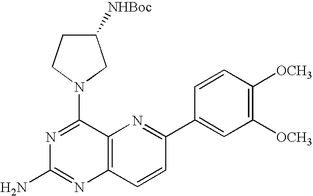

To a solution of 2-amino-4-[(S)-3-(Boc-amino)pyrrolidine]-6-chloro-pyrido[3,2-d]pyrimidine (100 mg, 0.27 mmol) in 1,4-dioxane (20 ml) and water (7 ml) was added 3,4-dimethoxyphenyl boronic acid (65 mg, 0.36 mmol), potassium carbonate (114 mg, 0.82 mmol) and tetrakis(triphenylphosphine)palladium(0) (16 mg, 0.014 mmol). The reaction mixture was refluxed for three hours, cooled down to room temperature and the solvents were evaporated in vacuo. The residue was purified by silica gel column chromatography, the mobile phase being a CH₃OH/dichloromethane mixture (in a ratio of 4:96), yielding the title compound as a pure white powder (79 mg, 63%).

MS (m/z): 467 ([M+H]⁺, 100).

### EXAMPLE 219

### Synthesis of 2-amino-4-[(S)-3-(amino)pyrrolidine]-6-(3,4-dimethoxyphenyl)-pyrido[3,2-d]pyrimidine

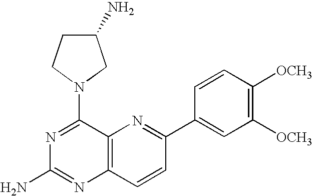

A solution of 2-amino-4-[(S)-3-(Boc-amino)pyrrolidine]-6-(3,4-dimethoxy-phenyl)-pyrido[3,2-d]pyrimidine (113 mg, 0.24 mmol) in dichloromethane (10 ml) and trifluoroacetic acid (4 ml) was stirred at room temperature for 30 minutes. The solvents were evaporated. The salt was redissolved in water and the solution was made alkaline (pH=9) by the addition of a 33% aqueous ammonia solution. The solvents were evaporated in vacuo and the residue was purified by silica gel flash chromatography, the mobile phase being a mixture of CH₃OH/CH₂Cl₂ in a ratio of 4:96, containing 0.5% of an aqueous 33% ammonia solution, yielding the title compound as a pure white solid (76 mg, 87%). MS (m/z): 367 ([M+H]⁺, 100).

### EXAMPLE 220

### Synthesis of 2-amino-4-[3-(S)-4-chloro-phenoxy-acetyl-amino)-pyrrolidin-1-yl]-6-(3,4-dimethoxyphenyl)-pyrido[3,2-d]pyrimidine

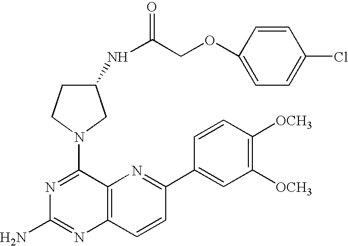

To a solution of 2-amino-4-[(S)-3-(amino)pyrrolidine]-6-(3,4-dimethoxyphenyl)-pyrido[3,2-d]pyrimidine (76 mg, 0.21 mmol) in DMF (10 ml) was added triethylamine (38 μl, 0.27 mmol) and p-chloro-phenoxy acetyl chloride (51 mg, 0.25 mmol). The reaction was stirred at 60° C. for 2 hours. The solvents were evaporated in vacuo and the crude residue was purified by silica gel flash chromatography, the mobile phase being a mixture of CH₃OH/CH₂Cl₂ in a ratio of 4:96, yielding the pure title compound (87 mg, 78%). MS (m/z): 535, 537 ([M+H]⁺, 100).

### EXAMPLE 221

### Synthesis of 2-amino-4-[3-(S)-3-methyl phenyl carbamoyl pyrrolidin-1-yl]-6-(3,4-dimethoxyphenyl)-pyrido[3,2-d]pyrimidine

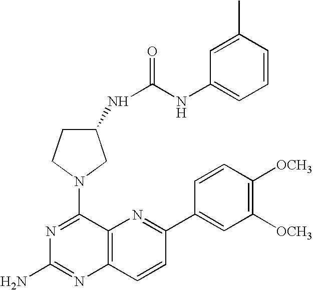

To a solution of 2-amino-4-[(S)-3-(amino)pyrrolidine]-6-(3,4-dimethoxyphenyl)-pyrido[3,2-d]pyrimidine (113 mg, 0.25 mmol) in dichloromethane (10 ml) was added m-tolyl isocyanate (0.28 mmol, 35 μl). The reaction was stirred at room temperature for 2 hours. The solvents were evaporated in vacuo and the crude residue was purified by silica gel flash chromatography, the mobile phase being a mixture of CH₃OH/CH₂Cl₂ in a ratio of 3:97, yielding the pure title compound (77 mg, 62%). MS (m/z): 500 ([M+H]⁺, 100).

### EXAMPLE 222

### Synthesis of 2-amino-6-(3,4-dimethoxyphenyl)-pyrido[3,2-d]pyrimidin-4(3H)thione

A suspension of 2-amino-6-(3,4-dimethoxyphenyl)-pyrido[3,2-d]pyrimidin-4(3H)one (100 mg, 0.34 mmol) and phosphorus pentasulfide (163 mg, 0.37 mmol) in pyridine (10 ml) was refluxed for 4 hours. The solvents were evaporated in vacuo. The residue was resuspended in a small amount of water and filtered off, yielding the title compound which was used without any further purification. MS (m/z): 315 ([M+H]⁺, 100).

### EXAMPLE 223

### Synthesis of 2-amino-4-thiomethyl-6-(3,4-dimethoxyphenyl)-pyrido[3,2-d]pyrimidine

The crude compound obtained in example 222 was dissolved in NaOH 1 N. Then, methyl iodide (18 μl, 0.29 mmol) was added and the reaction mixture was stirred at room temperature for 2 hours. Then, an additional amount of methyl iodide (9 μl) was added and the reaction was stirred for another hour at room temperature. A yellow precipitate was formed, which was filtered off. The precipitate was adsorbed on silica and purified by silica gel flash chromatography, the mobile phase being a methanol/dichloromethane mixture (in a ratio of 1:99), yielding the pure title compound (52 mg, 47%). MS (m/z): 329 ([M+H]⁺, 100).

### EXAMPLE 224

### Synthesis of 3-amino-6-chloro-pyridine-2-carbonitrile

To a suspension of 6-chloro-3-nitro-pyridine-2-carbonitrile (5.5 g, 30 mmol) in water (100 ml), was added acetic acid (5.4 ml, 90 mmol). The mixture was stirred at room temperature for 20 minutes. Then, Na₂S₂O₄ (20 g, 86%, 90 mmol) was added slowly. The reaction mixture was stirred at room temperature for another 2 hours. The precipitate was filtered off and washed with cold water (2×10 ml). The precipitate was dried over P₂O₅ yielding the title compound as a yellowish solid (3.7 g, 80%) which was characterised as follows:

Rf=0.64 (EtOAc/CH₂Cl₂1:4); and

MS (m/z): 154, 156 ([M+H]⁺, 100).

### EXAMPLE 225

### Synthesis of 2,4-diamino-6-chloro-pyrido[3,2-d]pyrimidine

A mixture consisting of 3-amino-6-chloro-pyridine-2-carbonitrile (4.6 g, 30 mmol), chloroformamidine hydrochloride (6.9 g, 60 mmol) and dimethylsulfon (12 g) was heated at 165° C. for 30 minutes. After cooling to room temperature, water (500 ml) was added. The solution was neutralized with a 30% NaOH solution to pH 9-10. The precipitate was filtered off, washed with water, dried over P₂O₅, yielding the title compound as a yellow solid (4.0 g, 68%) which was characterised as follows:

Rf=0.40 (MeOH/CH₂Cl₂ 1:9); and

MS (m/z): 196, 198 ([M+H]⁺, 100).

### EXAMPLE 226

### Synthesis of 3-amino-6-chloro-pyridine-2-carboxamide

To a suspension of 6-chloro-3-nitro-pyridine-2-carbonitrile (4 g, 22 mmol) in water (40 ml) was added a 33% aqueous solution of ammonia in water (8.8 ml). This suspension was stirred at room temperature for 30 minutes. Then, sodium dithionite (21.8 g, 124 mmol) was added portionwise. The resulting mixture was stirred for another 2 hours at room temperature. The precipitate was filtered off and washed with a small amount of water, yielding the title compound (2.7 g, 72%). MS (m/z): 172, 174 ([M+H]⁺, 100)

### EXAMPLE 227

### Synthesis of 2-amino-6-chloro-pyrido[3,2-d]pyrimidin-4(3H)one

### Method A

A suspension of 2,4-diamino-6-chloro-pyrido[3,2-d]pyrimidine (3.5 g, 17 mmol) in 5 N HCl (150 ml) was refluxed for 3 hours. After cooling to room temperature, the mixture was neutralized with a 30% NaOH solution to pH 6-7. The precipitate was filtered off, washed with water, dried over P₂O₅, yielding the title compound as a yellow solid (3.2 g, 90%).

### Method B

A mixture of 3-amino-6-chloro-pyridine-2-carboxamide (2.4 g, 14 mmol), chloro-formamidine hydrochloride (3.2 g, 28 mmol), dimethylsulfone (6 g) and sulfolane (0.8 ml) was heated at 165° C. for 30 minutes. After cooling to room temperature, water (600 ml) was added and the pH was adjusted to 7-8 with a 25% ammonia solution in water. The precipitate was filtered off, washed with water and dried over P₂O₅, yielding the title compound as a yellow solid (2.7 g, 98%) which was characterised as follows:

Rf=0.33 (MeOH/CH₂Cl₂1:4); and

MS (m/z): 197, 199 ([M+H]⁺, 100).

### EXAMPLE 228

### Synthesis of 2-acetamido-6-chloro-pyrido[3,2-d]pyrimidin-4(3H)one

A suspension of 2-amino-6-chloro-pyrido[3,2-d]pyrimidin-4(3H)one (3.2 g, 16 mmol) in acetic anhydride (20 ml) was refluxed for 2 hours. After cooling to room temperature, the precipitate was filtered off, washed with diethyl ether and dried under vacuum yielding the title compound as a yellowish solid (3.2 g, 85%) which was characterised as follows:

Rf=0.75 (MeOH/CH₂Cl₂ 1:4); and

MS (m/z): 238, 240 ([M+H]⁺, 100).

### EXAMPLE 229

### Synthesis of 2-acetamido-4-morpholino-6-chloro-pyrido[3,2-d]pyrimidine

A mixture of 2-acetamido-6-chloro-pyrido[3,2-d]pyrimidin-4(3H)one (2.4 g, 10 mmol), N,N-diisopropylethylamine (5.4 ml, 30 mmol) and POCl₃ (2.8 ml, 30 mmol) in dioxane (100 ml), was stirred at room temperature for 2 hours. After concentration under reduced pressure, the residue was redissolved in dichloromethane (200 ml) and extracted with cold water till pH 6-7. The combined organic layers were dried over MgSO₄, filtered and concentrated under reduced pressure to yield crude 2-acetamido-4,6-dichloro-pyrido[3,2-d]pyrimidine. This crude residue was dissolved in 1,4-dioxane (100 ml) and morpholine (5 ml) was added. The resulting reaction mixture was stirred at 50° C. for 1 hour. After concentration under reduced pressure, the residue was purified by silica gel flash chromatography, the mobile phase being a mixture of MeOH/dichloromethane (in a ratio of 1:40), yielding the title compound as a yellowish solid (1.6 g, 68%) which was characterised as follows:

Rf=0.82 (MeOH/CH₂Cl₂ 1:19); and

MS (m/z): 308, 310 ([M+H]⁺, 100).

### EXAMPLE 230

### Synthesis of 2-amino-6-chloro-4-morpholino-pyrido[3,2-d]pyrimidine

A suspension of 2-acetamido-4-morpholino-6-chloro-pyrido[3,2-d]pyrimidine (500 mg, 1.6 mmol) and K₂CO₃ (660 mg, 4.8 mmol) in MeOH (30 ml) and water (10 ml) was refluxed for 2 hours. After cooling to room temperature, the mixture was extracted with dichloromethane (100 ml), washed with water and dried over MgSO₄. After filtration and concentration, the residue was purified by silica gel flash chromatography, the mobile phase being a MeOH/CH₂Cl₂ mixture (in a ratio of 1:35) yielding the title compound as yellowish solid (425 mg, 98%) which was characterised as follows:

Rf=0.64 (MeOH/CH₂Cl₂ 1:9);

UV (MeOH/H₂O): 245, 330, and 455 nm; and

MS (m/z): 266, 268 ([M+H]⁺, 100)

### EXAMPLES 231 TO 246

### Synthesis of 2-amino-4-morpholino-6-aryl-pyrido[3,2-d]pyrimidine analogues and 2-amino-4-morpholino-6-heteroaryl-pyrido[3,2-d]pyrimidine analogues

To a solution of 2-amino-4-morpholino-6-chloro-pyrido[3,2-d]pyrimidine (53 mg, 0.2 mmol) in 1,4-dioxane (15 ml) and water (5 ml) was added an appropriate aryl or heteroaryl boronic acid (0.2 mmol), potassium carbonate (280 mg, 2 mmol) and tetrakis(triphenylphosphine)palladium(0) (30 mg, 0.026 mmol). The reaction mixture was refluxed for three hours, cooled down to room temperature and the solvents were evaporated in vacuo. The residue was purified by silica gel column chromatography, the mobile phase being a CH₃OH/dichloromethane mixture, thus resulting in the pure desired compounds in the following yields:

### EXAMPLE 231

### 2-amino-4-morpholino-6-(3,4-dichlorophenyl)-pyrido[3,2-d]pyrimidine

was obtained from 3,4-dichlorophenylboronic acid as a yellowish solid (79%) and was characterised as follows:

Rf=0.55 (MeOH/CH₂Cl₂ 1:9);

UV (MeOH/H₂O, nm): 283.8, 365.9; and

MS (m/z): 376, 378 ([M+H]⁺, 100).

### EXAMPLE 232

### 2-amino-4-morpholino-6-(2-furan)-pyrido[3,2-d]pyrimidine

Was obtained from 2-furanboronic acid as a yellow solid (79%) and was characterised as follows:

Rf=0.36 (MeOH/CH₂Cl₂ 1:9);

UV (MeOH/H₂O, nm): 212.9, 290.9, 377.9; and

MS (m/z): 298 ([M+H]⁺, 100).

### EXAMPLE 233

### 2-amino-4-morpholino-6-(3-thiophene)-pyrido[3,2-d]pyrimidine

Was obtained from 3-thiopheneboronic acid as a yellowish solid (73%) and was characterised as follows:

Rf=0.50 (MeOH/CH₂Cl₂ 1:9);

UV (MeOH/H₂O, nm): 215.3, 279.1, 362.5; and

MS (m/z): 314 ([M+H]⁺, 100).

### EXAMPLE 234

### 2-amino-4-morpholino-6-(4-pyridinyl)-pyrido[3,2-d]pyrimidine

Was obtained from 4-pyridine boronic acid as a yellowish solid (90%) and was characterised as follows:

Rf=0.63 (MeOH/CH₂Cl₂ 1:9);

UV (MeOH/H₂O, nm): 214.1, 236.5, 280.3, 341, 356.6; and

MS (m/z): 309 ([M+H]⁺, 100)

### EXAMPLE 235

### 2-amino-4-morpholino-6-(5-methyl-2-thienyl)-pyrido[3,2-d]pyrimidine

Was obtained from 5-methyl-2-thiophene boronic acid as a yellowish solid (69%) and was characterised as follows:

Rf=0.60 (MeOH/CH₂Cl₂ 1:9);

UV (MeOH/H₂O, nm): 214.1, 298.1, 380.3; and

MS (m/z): 328 ([M+H]⁺, 100).

### EXAMPLE 236

### 2-amino-4-morpholino-6-(6-methoxy-2-pyridinyl)-pyrido[3,2-d]pyrimidine

Was obtained from 6-methoxy-2-pyridine boronic acid as a yellowish solid (75%) and was characterised as follows:

Rf=0.44 (MeOH/CH₂Cl₂ 1:9);

UV (MeOH/H₂O, nm): 214.1, 283.8, 359.5; and

MS (m/z): 339 ([M+H]⁺, 100).

### EXAMPLE 237

### 2-amino-4-morpholino-6-(5-indolyl)-pyrido[3,2-d]pyrimidine

Was obtained from 5-indole boronic acid as a yellowish solid (90%) and was characterised as follows:

Rf=0.25 (MeOH/CH₂Cl₂ 1:9);

UV (MeOH/H₂O, nm): 216.5, 314.7, 422.5, 441.9; and

MS (m/z): 347 ([M+H]⁺, 100).

### EXAMPLE 238

### 2-amino-4-morpholino-6-(2-thienyl)-pyrido[3,2-d]pyrimidine

Was obtained from 2-thiophene boronic acid as a yellowish solid (72%) and was characterised as follows:

Rf=0.70 (MeOH/CH₂Cl₂ 1:9);

UV (MeOH/H₂O, nm): 214.1, 293.3, 377.9; and

MS (m/z): 314 ([M+H]⁺, 100).

### EXAMPLE 239

### 2-amino-4-morpholino-6-(4-methyl-2-thienyl)-pyrido[3,2-d]pyrimidine

Was obtrained from 4-methyl-2-thiophene boronic acid as a yellowish solid (76%) and was characterised as follows:

Rf=0.45 (MeOH/CH₂Cl₂ 1:9);

UV (MeOH/H₂O, nm): 212.9, 298.1, 380.3;

MS (m/z): 328 ([M+H]⁺, 100).

### EXAMPLE 240

### 2-amino-4-morpholino-6-(3-pyridinyl)-pyrido[3,2-d]pyrimidine

Was obtained from 3-pyridine boronic acid as a yellowish solid (90%) and was characterised as follows:

Rf=0.55 (MeOH/CH₂Cl₂ 1:9);

UV (MeOH/H₂O, nm): 214.1, 247.1, 285, 363.5; and

MS (m/z): 309 ([M+H]⁺, 100).

### EXAMPLE 241

### 2-amino-4-morpholino-6-(5-chloro-2-thienyl)-pyrido[3,2-d]pyrimidine

Was obtained from 5-chloro-2-thiophene boronic acid as a yellowish solid (29%) and was characterised as follows:

Rf=0.65 (MeOH/CH₂Cl₂ 1:9);

UV (MeOH/H₂O, nm): 212.9, 298.1, 380.3; and

MS (m/z): 348 ([M+H]⁺, 100).

### EXAMPLE 242

### 2-amino-4-morpholino-6-(3-chloro-4-fluorophenyl)-pyrido[3,2-d]pyrimidine

Was obtained from 3-chloro-4-fluorophenyl boronic acid as a yellowish solid (75%) and was characterised as follows:

Rf=0.55 (MeOH/CH₂Cl₂ 1:9);

UV (MeOH/H₂O, nm): 345, 480, 560; and

MS (m/z): 360 ([M+H]⁺, 100).

### EXAMPLE 243

### 2-amino-4-morpholino-6-(3,4-difluorophenyl)-pyrido[3,2-d]pyrimidine

Was obtained from 3,4-difluorophenyl boronic acid as a yellowish solid (75%) and was characterised as follows:

Rf=0.64 (MeOH/CH₂Cl₂ 1:9);

UV (MeOH/H₂O, nm): 345, 465, 560; and

MS (m/z): 344 ([M+H]⁺, 100).

### EXAMPLE 244

### 2-amino-4-morpholino-6-(4-fluoro-3-methylphenyl)-pyrido[3,2-d]pyrimidine

Was obtained from 4-fluoro-3-methylphenyl boronic acid as a white solid (81%) and was characterised as follows:

Rf=0.60 (MeOH/CH₂Cl₂ 1:9);

UV (MeOH/H₂O, nm): 280.3, 365.9; and

MS (m/z): 340 ([M+H]⁺, 100).

### EXAMPLE 245

### 2-amino-4-morpholino-6-(4-fluorophenyl)-pyrido[3,2-d]pyrimidine

Was obtained from 4-fluorophenyl boronic acid as a white solid (85%) and was characterised as follows:

Rf=0.64 (MeOH/CH₂Cl₂1:9);

UV (MeOH/H₂O, nm): 250, 470, 560; and

MS (m/z): 326 ([M+H]⁺, 100).

### EXAMPLE 246

### 2-amino-4-morpholino-6-[4-(3,5-dimethylisoxazolyl)]-pyrido[3,2-d]pyrimidine

Was obtained from 3,5-dimethylisoxazole-4-boronic acid as a yellowish solid (62%) and was characterised as follows:

Rf 0.60 (MeOH/CH₂Cl₂ 1:9);

UV (MeOH/H₂O, nm): 214.1, 269.6, 356.6; and

MS (m/z): 327 ([M+H]⁺, 100).

### EXAMPLE 247

### Synthesis of 2-acetamido-4-(N-homopiperazin-1-yl)-6-chloro-pyrido[3,2-d]pyrimidine

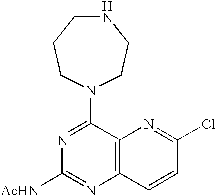

This compound was synthesized from homopiperazine according to the procedure of example 229, yielding the pure title compound as a yellowish solid (49%) which characterised as follows:

Rf=0.17 (MeOH/CH₂Cl₂ 1:4); and

MS (m/z): 321, 323 ([M+H]⁺, 100).

### EXAMPLE 248

### Synthesis of 2-acetamido-4-[(N-3-methylphenylcarbamoyl)-homopiperazin-1-yl]-6-chloro-pyrido[3,2-d]pyrimidine

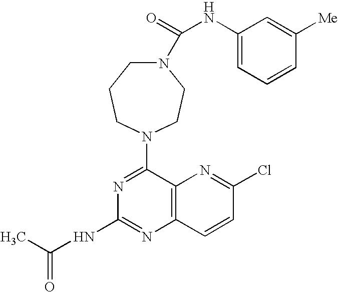

To a solution of 2-acetamido-6-chloro-4-(N-homopiperazin-1-yl)-pyrido[3,2-d]pyrimidine (95 mg, 0.3 mmol) in dichloromethane (10 ml) was added m-tolylisocyanate (40 mg, 0.3 mmol). The solution was stirred at room temperature for 1 hour. The solvents were evaporated in vacuo yielding the crude title compound, which was used for further reaction without any purification.

### EXAMPLE 249

### Synthesis of 2-acetamido-4-[(N-3-methylphenylcarbamoyl)-homopiperazin-1-yl]-6-(3,4-dimethoxyphenyl)-pyrido[3,2-d]pyrimidine

To a solution of crude 2-acetamido-6-chloro-4-[N-(3-methylphenylcarbamoyl)-homopiperazin-1-yl]-pyrido[3,2-d]pyrimidine (130 mg, 0.3 mmol) in dioxane (15 ml) and water (5 ml) was added 3,4-dimethoxyphenyl boronic acid (55 mg, 0.3 mmol), potassium carbonate (280 mg, 2 mmol) and tetrakis(triphenylphosphine)palladium(0) (30 mg, 0.026 mmol). The reaction mixture was refluxed for 30 minutes. The solvents were evaporated in vacuo. The crude residue was purified by silica gel flash chromatography, the mobile phase being a MeOH/CH₂Cl₂ mixture (in a ratio of 1:40), yielding the pure title compound (126 mg, 78%). MS (m/z): 556 ([M+H]⁺, 100).

### EXAMPLE 250

### Synthesis of 2-amino-4-[(N-3-methylphenylcarbamoyl)-homopiperazin-1-yl]-6-(3,4-dimethoxyphenyl)-pyrido[3,2-d]pyrimidine

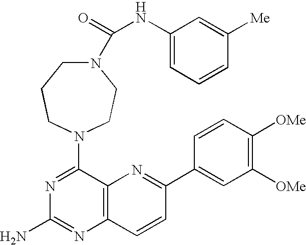

A solution of 2-acetamido-4-[(N-3-methylphenylcarbamoyl)-homopiperazin-1-yl]-6-(3,4-dimethoxyphenyl)-pyrido[3,2-d]pyrimidine (110 mg, 0.24 mmol) and potassium carbonate (83 mg, 0.6 mmol) in methanol (10 ml) and water (5 ml) was heated at 50° C. for 2 hours. The solvents were evaporated in vacuo and the crude residue was purified by silica gel flash chromatography, the mobile phase being a MeOH/CH₂Cl₂ mixture in a volume ratio of 1:30, yielding the pure title compound (96 mg, 93%) which characterised as follows:

Rf=0.55 (MeOH/CH₂Cl₂ 1/9);

UV (MeOH/H₂O, nm): 245, 490, 565; and

MS (m/z): 514 ([M+H]⁺, 100).

### EXAMPLE 251

### Synthesis of 2-acetamido-4-[(R)-3-Boc-aminopyrrolidin-1-yl]-6-chloro pyrido[3,2-d]pyrimidine

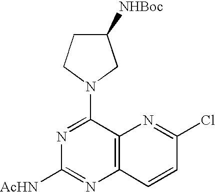

This compound was prepared from (R)-3-Boc-amino-pyrrolidine according to the procedure of example 229, yielding the title compound as a yellowish solid (46%) which characterised as follows:

Rf=0.55 (MeOH/CH₂Cl₂ 1:9); and

MS (m/z): 407, 409 ([M+H]⁺, 100).

### EXAMPLE 252

### Synthesis of 2-amino-4-[(R)-3-Boc-aminopyrrolidin-1-yl)]-6-(3,4-dimethoxyphenyl)-pyrido[3,2-d]pyrimidine

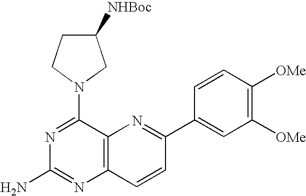

This compound was synthesized from the compound of example 251. In a first step, a Suzuki coupling with 3,4-dimethoxyphenyl boronic acid (general procedure as in examples 231 to 246) was performed. In a second step, alkaline hydrolysis of the acetyl group (using the procedure for the synthesis of example 230) yielded the pure title compound (81%) which characterised as follows:

Rf=0.54 (MeOH/CH₂Cl₂ 1:9);

UV (MeOH/H₂O, nm): 280, 470, 565; and

MS (m/z): 467 ([M+H]⁺, 100).

### EXAMPLE 253 TO 258

### Synthesis of 2-amino-4-substituted-6-(3,4-dimethoxyphenyl)-Pyrido[3,2-d]pyrimidines

A suspension of 2-amino-6-(3,4-dimethoxyphenyl)-pyrido[3,2-d]pyrimidin-4(3H)one (298 mg, 1.0 mmol), 1,1,1,3,3,3-hexamethyldisilazane (1 ml, 4.7 mmol), an appropriate amine (4.0 mmol), p-toluenesulfonic acid (20 mg, 0.1 mmol) and ammonium sulfate (20 mg, 0.15 mmol) in pyridine (5 ml) was refluxed for 12 to 48 hours (depending upon the amine used; the reaction mixture became clear when reaction was completed). The solvents were evaporated in vacuo and the residue was purified by silica gel flash chromatography, the mobile phase being a MeOH/dichloromethane mixture (in a volume ratio of 1:20 to 1:30, depending upon the amine used), resulting into the title compounds as yellow solids in the following yields.

### EXAMPLE 253

### 2-amino-4-(ethylenediamino-1-N-yl)-6-(3,4-dimethoxyphenyl)-pyrido[3,2-d]pyrimidine

Was obtained from ethylene diamine as a yellowish solid (64%) which characterised as follows:

Rf=0.25 (MeOH/CH₂Cl₂ 1:4); and

MS (m/z): 341 ([M+H]⁺, 100).

### EXAMPLE 254

### 2-amino-4-(1,3-diaminopropane-1-N-yl]-6-(3,4-dimethoxyphenyl)-Pyrido[3,2-d]pyrimidine

Was obtained from 1,3-diaminopropane as a yellowish solid (68%) which characterised as follows:

Rf=0.28 (MeOH/CH₂Cl₂ 1:4); and

MS (m/z): 355 ([M+H]⁺, 100).

### EXAMPLE 255

### 2-amino-4-[(1-Boc-piperidin-4-yl)amino]-6-(3,4-dimethoxyphenyl)-pyrido[3,2-d]pyrimidine

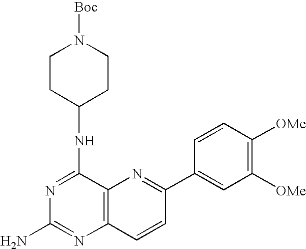

was obtained from 4-amino-N-Boc-piperidine as a yellowish solid (92%) which characterised as follows:

Rf=0.58 (MeOH/CH₂Cl₂ 1:9);

UV (MeOH/H₂O, nm): 250, 480, 565; and

MS (m/z): 481 ([M+H]⁺, 100).

### EXAMPLE 256

### 2,4-diamino-6-(3,4-dimethoxyphenyl)-pyrido[3,2-d]pyrimidine

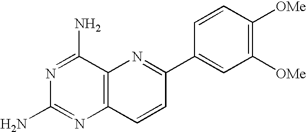

was obtained from ammonium chloride as a yellowish solid (56%) which characterised as follows:

Rf=0.23 (MeOH/CH₂Cl₂ 1:4);

UV (MeOH/H₂O, nm): 245, 585; and

MS (m/z): 298 ([M+H]⁺, 100).

### EXAMPLE 257

### 2-amino-4-[(1-Boc-piperidin-3-yl)amino]-6-(3,4-dimethoxyphenyl)-pyrido[3,2-d]pyrimidine

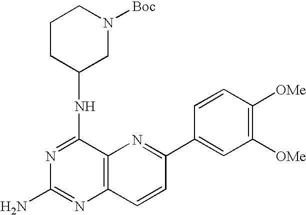

was obtained from 3-amino-N-Boc-piperidine as a yellowish solid (70%) which characterised as follows:

Rf=0.60 (MeOH/CH₂Cl₂ 1:9);

UV (MeOH/H₂O, nm): 250, 490, 565; and

MS (m/z): 481 ([M+H]⁺, 100).

### EXAMPLE 258

### 2-amino-4-[(1-Cbz-piperidin-3-yl)amino]-6-(3,4-dimethoxyphenyl)-pyrido[3,2-d]pyrimidine

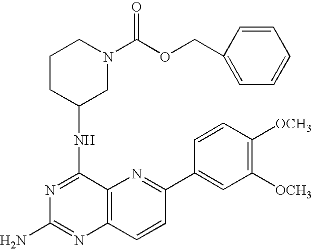

was synthesized from 3-amino-1-benzyloxycarbonyl-piperidine, yielding the title compound (63%). MS (m/z): 515 ([M+H]⁺, 100).

### EXAMPLE 259

### Synthesis of 2-amino-4-[(R)-3-aminopyrrolidin-1-yl]-6-(3,4-dimethoxy-phenyl)-pyrido[3,2-d]pyrimidine

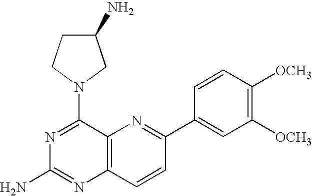

To a suspension of 2-amino-4-[(R)-3-Boc-aminopyrrolidin-1-yl]-6-(3,4-di-methoxyphenyl)pyrido[3,2-d]pyrimidine (94 mg, 0.2 mmol) in dichloromethane (5 ml) was added trifluoroacetic acid (2 ml). The resulting solution was stirred at room temperature for 30 minutes. The solvents were removed under reduced pressure. The residue was extracted with chloroform and washed with a 0.2 M Na₂CO₃ solution. The combined organic layers were evaporated in vacuo. The crude residue was purified by silica gel flash chromatography, the mobile phase being a MeOH/CH₂Cl₂ mixture in a volume ratio of 2:3, yielding the pure title compound (70 mg, 96%). MS (m/z): 367 ([M+H]⁺, 100).

### EXAMPLE 260

### Synthesis of 2-amino-4-[3-(R)-(3-methylphenylcarbamoyl)-pyrrolidin-1-yl]-6-(3,4-dimethoxyphenyl)-pyrido[3,2-d]pyrimidine

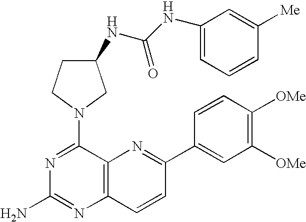

To a solution of 2-amino-4-[(R)-3-Boc-aminopyrrolidin-1-yl)]-6-(3,4-dimethoxy-phenyl)-pyrido[3,2-d]pyrimidine (55 mg, 0.12 mmol) in dichloromethane (5 ml) was added trifluoroacetic acid (2 ml). The mixture was stirred at room temperature for 30 minutes. The solvents were evaporated in vacuo. To a suspension of this crude residue in dichloromethane (5 ml) was added N,N-diisopropylethylamine (0.5 ml) and m-tolyl isocyanate (16 μl). The reaction mixture was stirred at room temperature for 30 minutes. The solvents were evaporated in vacuo. The crude residue was purified by silica gel chromatography, the mobile phase being a MeOH/CH₂Cl₂ mixture (in a ratio of 1:20), yielding the pure title compound (50 mg, 85%) which characterised as follows:

Rf=0.42 (MeOH/CH₂Cl₂ 1:9);

UV (MeOH/H₂O, nm): 240, 470, 560; and

MS (m/z): 500 ([M+H]⁺, 100).

### EXAMPLE 261

### Synthesis of 2-amino-4-[(3-methylphenylcarbamoyl)-ethylenediamine-1-N-yl]-6-(3,4-dimethoxyphenyl)-pyrido[3,2-d]pyrimidine

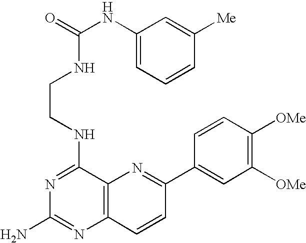

To a solution of 2-amino-4-(ethylenediamine-1-N-yl)-6-(3,4-dimethoxyphenyl)-pyrido[3,2-d]pyrimidine (70 mg, 0.2 mmol) in dichloromethane (10 ml) was added N,N-diisopropylethylamine (200 μl) and m-tolyl isocyanate (26 μl). The solution was stirred at room temperature for 1 hour. The solvents were evaporated in vacuo. The crude residue was purified by silica gel flash chromatography, the mobile phase being a MeOH/CH₂Cl₂ mixture, in a ratio of 1:15, yielding the pure title compound (72 mg, 76%) which characterised as follows:

Rf=0.32 (MeOH/CH₂Cl₂ 1:9);

UV (MeOH/H₂O, nm): 250, 560; and

MS (m/z): 474 ([M+H]⁺, 100).

### EXAMPLE 262

### Synthesis of 2-amino-4-[(3-methylphenylcarbamoyl)-3-aminopropane-amino-1-N-yl]-6-(3,4-dimethoxyphenyl)-pyrido[3,2-d]pyrimidine

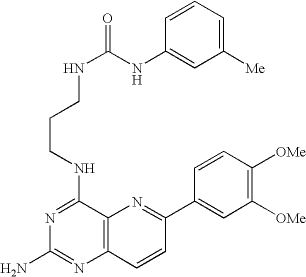

This compound was obtained from 2-amino-4-(3-aminopropanamine-1-N-yl)-6-(3,4-dimethoxyphenyl)-pyrido[3,2-d]pyrimidine and m-tolyl isocyanate (using the procedure described for the synthesis of example 261) in 82% yield and was characterised as follows:

Rf=0.38 (MeOH/CH₂Cl₂ 1:9);

UV (MeOH/H₂O, nm): 250, 480, 560; and

MS (m/z): 488 ([M+H]⁺, 100).

### EXAMPLE 263

### Synthesis of 2-amino-4-[1-(3-methylphenylcarbamoyl)piperidin-4-yl)amino]-6-(3,4-dimethoxyphenyl)-pyrido[3,2-d]pyrimidine

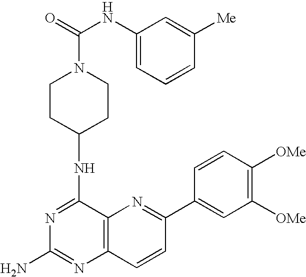

This compound was obtained from 2-amino-4-(1-Boc-piperidin-4-yl-amino)-6-(3,4-dimethoxyphenyl)-pyrido[3,2-d]pyrimidine as a yellowish solid (82%) which characterised as follows:

Rf=0.40 (MeOH/CH₂Cl₂ 1:9);

UV (MeOH/H₂O, nm): 250, 470, 560; and

MS (m/z): 514 ([M+H]⁺, 100).

### EXAMPLE 264

### Synthesis of 2-amino-4-[(3-methylphenylcarbamoylpiperidin-3-yl)amino)-6-(3,4-dimethoxyphenyl]-pyrido[3,2-d]pyrimidine

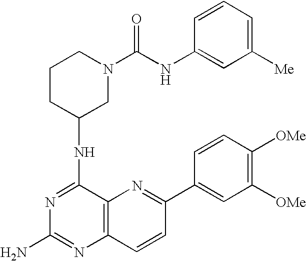

This compound was synthesized from 2-amino-4-(1-Boc-piperidin-3-ylamino)-6-(3,4-dimethoxyphenyl)-pyrido[3,2-d]pyrimidine by Boc-deprotection and coupling with m-tolyl isocyanate (using the procedure described for example 260), as a yellowish solid (88%) which was characterised as follows:

Rf=0.32 (MeOH/CH₂Cl₂ 1:9);

UV (MeOH/H₂O, nm): 250, 370, 560; and

MS (m/z): 514 ([M+H]⁺, 100).

### EXAMPLE 265

### Synthesis of 2-amino-4-[2-(4-chlorophenoxy-acetyl-ethylenediamine-1-N-yl]-6-(3,4-dimethoxyphenyl)-pyrido[3,2-d]pyrimidine

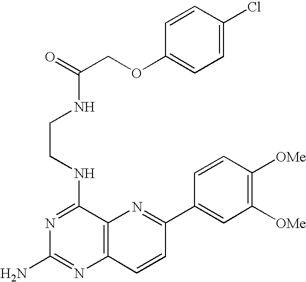

To a suspension of 2-amino-4-(ethylenediamine-1-N-yl)-6-(3,4-dimethoxy-phenyl)-pyrido[3,2-d]pyrimidine (50 mg, 0.15 mmol) in dichloromethane (10 ml) was added DIPEA (200 μl) and 4-chloro-phenoxy acetyl chloride (30 mg, 0.15 mmol). The mixture was stirred at room temperature for 1 hour. The solvents were evaporated in vacuo. The crude residue was purified by flash chromatography, the mobile phase being a MeOH/CH₂Cl₂ mixture (in a ratio of 1:20), yielding the pure title compound (40 mg, 53%) which was characterised as follows:

Rf=0.35 (MeOH/CH₂Cl₂ 1:9);

UV (MeOH/H₂O, nm): 250, 480, 560; and

MS (m/z): 509, 511 ([M+H]⁺, 100).

### EXAMPLE 266

### Synthesis of 2-amino-4-[3-N-(4-chlorophenoxy-acetyl)-3-amino-propane-amine-1-N-yl]-6-(3,4-dimethoxyphenyl)-pyrido[3,2-d]pyrimidine

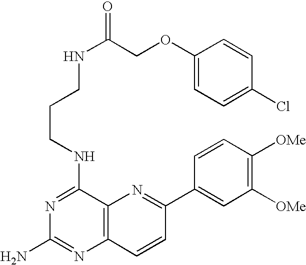

This compound was synthesized from 2-amino-4-(3-aminopropanamine-1-N-yl)-6-(3,4-dimethoxyphenyl)-pyrido[3,2-d]pyrimidine and 4-chlorophenoxyacetyl chloride, using the procedure described for the synthesis of example 265, yielding the pure title compound (56%) which was characterised as follows:

Rf=0.36 (MeOH/CH₂Cl₂ 1:9);

UV (MeOH/H₂O, nm): 250, 560; and

MS (m/z): 523, 525 ([M+H]⁺, 100).

### EXAMPLE 267

### Synthesis of 2-amino-4-[(3-(R)-(4-chlorophenoxyacetyl-amino)-pyrrolidin-1-yl]-6-(3,4-dimethoxyphenyl)-pyrido[3,2-d]pyrimidine

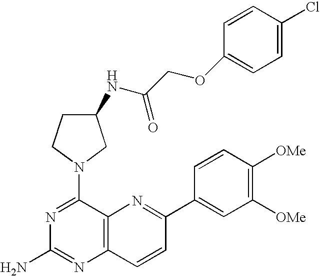

This compound was synthesized from 2-amino-4-[(3-(R)-Boc-aminopyrrolidin-1-yl]-6-(3,4-dimethoxyphenyl)-pyrido[3,2-d]pyrimidine in two steps. The Boc group was deprotected (using the procedure described for example 259) and then, the free amino group was coupled with 4-chlorophenoxyacetyl chloride (using the procedure described for example 265), yielding the pure title compound (68%) which was characterised as follows:

Rf=0.30 (MeOH/CH₂Cl₂ 1:9);

UV (MeOH/H₂O, nm): 280, 470, 560; and

MS (m/z): 535, 537 ([M+H]⁺, 100).

### EXAMPLES 268 TO 276

### Synthesis of 2-amino-4-substituted-6-(314-dimethoxyphenyl)-pyrido[3,2-d]pyrimidines

To a suspension of 2-acetamido-4-(1,2,4-triazolyl)-6-(3,4-dimethoxyphenyl)-pyrido[3,2-d]pyrimidine (0.5 mmol) and N,N-diisopropylethylamine (3 mmol) in 1,4-dioxane (20 ml) was added an appropriate amine (1.5 mmol). The reaction mixture was refluxed for 2 hours. The solvents were evaporated in vacuo and the residue was redissolved in methanol (20 ml). A solution of K₂CO₃ (3 mmol) in water (5 ml) was added and the resulting reaction mixture was refluxed for 2 hours. After cooling to room temperature, the mixture was extracted with dichloromethane (100 ml). The organic phase was washed with a 0.5 M Na₂CO₃ solution and concentrated under reduced pressure. The residue was purified by silica gel flash chromatography, the mobile phase being a mixture of MeOH and dichloromethane, thus resulting into the pure title compounds in the following yields.

### EXAMPLE 268

### 2-amino-4-[(3-carboxylic acid isobutylamide)-piperidin-1-yl]-6-(3,4-dimethoxyphenyl)-pyrido[3,2-d]pyrimidine

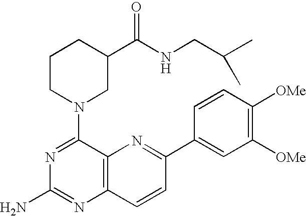

This compound was obtained from piperidine-3-carboxylic acid isobutyl amide, as a yellowish solid (60%) which was characterised as follows:

Rf=0.25 (MeOH/CH₂Cl₂ 1:9);

UV (MeOH/H₂O, nm): 245, 560; and

MS (m/z): 465 ([M+H]⁺, 100).

### EXAMPLE 269

### 2-amino-4-(4-chlorophenyl-4-hydroxypiperidin-1-yl)-6-(3,4-dimethoxy-phenyl)-pyrido[3,2-d]pyrimidine

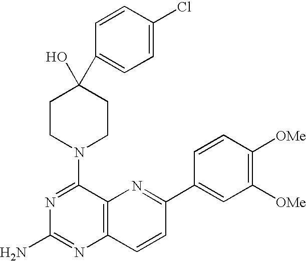

This compound was obtained from 4-(4-chlorophenyl)-4-hydroxy-piperidine, as a white solid (58%) which was characterised as follows:

Rf=0.42 (MeOH/CH₂Cl₂ 1:9);

UV (MeOH/H₂O, nm): 285, 365, 560; and

MS (m/z): 492, 494 ([M+H]⁺, 100).

### EXAMPLE 270

### 2-amino-4-[4-(N-2-phenylethylacetamid-2-yl)piperazin-1-yl]-6-(3,4-dimethoxyphenyl)-pyrido[3,2-d]pyrimidine

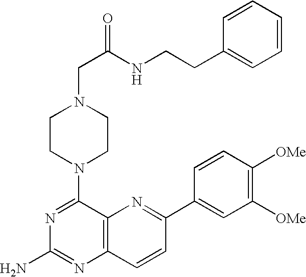

This compound was synthesized from N-(2-phenylethyl)-2-piperazin-1-yl-acetamide as a yellowish solid (54%) which was characterised as follows:

Rf=0.38 (MeOH/CH₂Cl₂ 1:9);

UV (MeOH/H₂O, nm): 245, 560; and

MS (m/z): 528 ([M+H]⁺, 100).

### EXAMPLE 271

### 2-amino-4-[2-(4-benzylpiperazin-1-yl)-2-oxo-ethane-amino]-6-(3,4-dimethoxyphenyl)-pyrido[3,2-d]pyrimidine

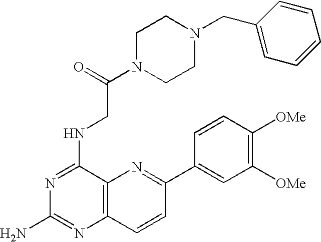

This compound was obtained from 2-amino-1-(4-benzylpiperazin-1-yl)-ethanone as a yellowish solid (54%) which was characterised as follows:

Rf=0.32 (MeOH/CH₂Cl₂ 1:9);

UV (MeOH/H₂O, nm): 265, 585;

MS (m/z): 514 ([M+H]⁺, 100)

### EXAMPLE 272

### 2-amino-4-[3-(4-acetylpiperazin-1-yl)-propan-3-one-1-yl-amino]-6-(3,4-dimethoxyphenyl)-pyrido[3,2-d]pyrimidine

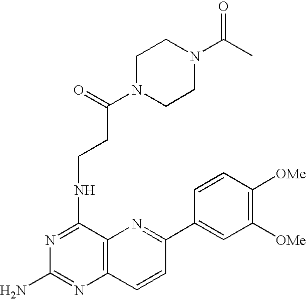

This compound was obtained from 1-(4-acetylpiperazin-1-yl)-3-aminopropan-1-one as a yellowish solid (60%) which was characterised as follows:

Rf=0.30 (MeOH/CH₂Cl₂ 1:9);

UV (MeOH/H₂O, nm): 250, 505, 580; and

MS (m/z): 480 ([M+H]⁺, 100)

### EXAMPLE 273

### 2-amino-4-(N-pyrrolidinyl-acetamid-2-yl-piperazin-1-yl)-6-(3,4-dimethoxyphenyl)pyrido[3,2-d]pyrimidine

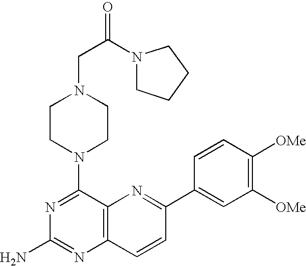

This compound was synthesized from 2-piperazine-1-yl-1-pyrrolidin-1-yl-ethanone (yield 59%) as a yellowish solid which was characterised as follows:

Rf=0.27 (MeOH/CH₂Cl₂ 1/9);

UV (MeOH/H₂O, nm): 245, 580; and

MS (m/z): 478 ([M+H]⁺, 100)

### EXAMPLE 274

### Synthesis of 2-amino-4-(N-pyridinyl-acetamid-2-Vi-piperazin-1-yl)-6-(3,4-dimethoxyphenyl)-pyrido[3,2-d]-pyrimidine

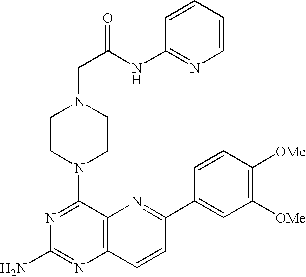

This compound was synthesized from 2-piperazin-1-yl-N-pyridin-2-yl-acetamide as a yellowish solid (53%) which was characterised as follows:

Rf=0.33 (MeOH/CH₂Cl₂1:9);

UV (MeOH/H₂O, nm): 245, 365, 560; and

MS (m/z): 501 ([M+H]⁺, 100)

### EXAMPLE 275

### 2-amino-4-[N-(piperazino)-acetyl-morpholino]-6-(3,4-dimethoxyphenyl)-pyrido[3,2-d]pyrimidine

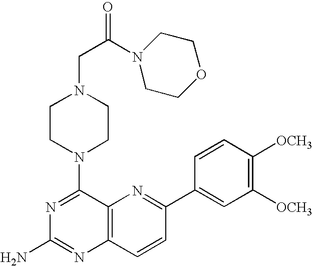

This compound was synthesized from N-[2-(1-piperazino)-acetyl]-morpholino as a yellowish solid (57%) which was characterised as follows:

Rf=0.45 (MeOH/CH₂Cl₂1:9);

UV (MeOH/H₂O, nm): 275, 365; and

MS (m/z): 494 ([M+H]⁺, 100)

### EXAMPLE 276

### 2-amino-4-[2-amino-1-(4-methyl-piperazin-1-yl)-ethanone]-6-(3,4-dimethoxyphenyl)-pyrido[3,2-d]pyrimidine

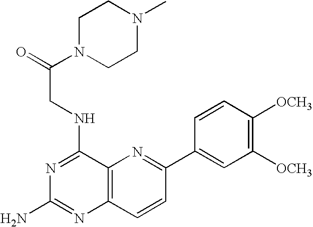

This compound was synthesized from 2-amino-1-(4-methylpiperazin-1-yl)-ethanone as a yellowish solid (57%) which was characterised as follows:

Rf=0.20 (MeOH/CH₂Cl₂ 1:4);

UV (MeOH/H₂O, nm): 270, 355, 495;

MS (m/z): 438 ([M+H]⁺, 100)

### EXAMPLES 277 AND 278

### Synthesis of 2-acetamido-4-substituted-6-(3,4-dimethoxy-phenyl)-pyrido[3,2-d]pyrimidine analogues

To a suspension of 2-acetamido-4-(1,2,4-triazolyl)-6-(3,4-dimethoxyphenyl)-pyrido[3,2-d]pyrimidine (0.5 mmol) and N,N-diisopropylethylamine (3 mmol) in 1,4-dioxane (20 ml) was added an appropriate amine (1.5 mmol). The reaction mixture was refluxed for 2 hours. The solvents were evaporated in vacuo and the residue was purified by silica gel chromatography, the mobile phase being a mixture of methanol and dichloromethane (in a ratio of 1:30) yielding the pure final compounds as follows:

### EXAMPLE 277

### 2-acetamido-4-[(N-pyridin-3-yl-acetamid)-2-yl-piperazin-1-yl]-6-(3,4-dimethoxyphenyl)-pyrido[3,2-d]pyrimidine

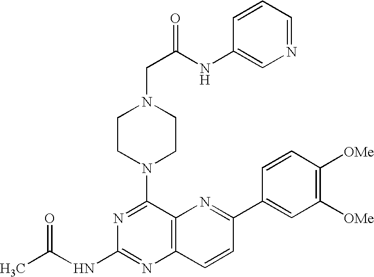

This compound was synthesized from 2-piperazin-1-yl-N-pyridin-3-yl-acetamide as a yellowish solid (40%) which was characterised as follows:

Rf=0.40 (MeOH/CH₂Cl₂ 1:9);

UV (MeOH/H₂O, nm): 245, 370; and

MS (m/z): 543 ([M+H]⁺, 100)

### EXAMPLE 278

### 2-acetamido-4-[(N-methyl-N-phenylacetamid)-2-yl-piperazin-1-yl]-6-(3,4-dimethoxyphenyl)-pyrido[3,2-d]pyrimidine

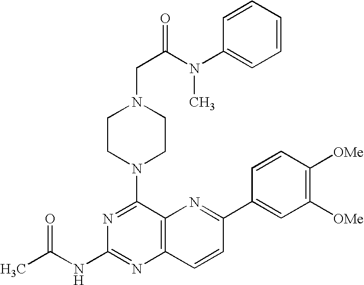

This compound was synthesized from N-methyl-N-phenyl-2-piperazin-1-yl-acetamide as a yellowish solid (38%) which was characterised as follows:

Rf=0.45 (MeOH/CH₂Cl₂ 1:9);

UV (MeOH/H₂O, nm): 255, 360; and

MS (m/z): 556 ([M+H]⁺, 100)

### EXAMPLE 279

### Synthesis of 3-amino-6-chloro-pyridine-2-carboxamide

To a suspension of 6-chloro-3-nitro-pyridine-2-carbonitrile (11.01 g, 60 mmol) in methanol (120 ml), was added Raney-Nickel (3 g, washed with methanol to remove water) and the mixture was shaken under a H₂-atmosphere at room temperature for 4 hours. The catalyst was removed by filtration, washed with methanol (500 ml). Both filtrates were combined and then evaporated to dryness. The residue was dissolved in dichloromethane and the solution was filtered through a short and wide column with silica gel (100 g). The column was additionally washed with CH₂Cl₂/MeOH (200 ml, 4:1). The filtrate and washings were combined and evaporated to small volume. The formed precipitate was filtered off to give 3-amino-6-chloro-pyridine-2-carboxamide (8.1 g). The final filtrate was evaporated to dryness and the residue purified by column chromatography on silica gel (30 g). The compound was eluted with the following solvent systems: CH₂Cl₂ (200 ml), CH₂Cl₂/MeOH 100:1 (200 ml). The appropriate fractions were evaporated in vacuo yielding an additional 1.15 g of 3-amino-6-chloro-pyridine-2-carboxamide (total yield: 9.25 g, i.e. 90%) which was characterised as follows:

M.p. 176-177° C.;

UV (MeOH): 212 (3.76), 256 (4.14), 348 (3.76); and
Elemental analysis: calculated for C₆H₆ClN₃O (171.6): C 42.00H 3.52 N 24.49. Found: C 42.42H 3.54H 24.11.

### EXAMPLE 280

### Synthesis of 2-amino-6-chloro-pyrido[3,2-d]pyrimidin-4(3H)-one

A mixture of 3-amino-6-chloro-pyridine-2-carboxamide (5.1 g, 30 mmol), chloroform-amidine hydrochloride (6.99 g, 60 mmol), dimethylsulfone (24 g) and sulfolane (2.4 ml) was heated at 165° C. for 30 min. To the hot mixture was added water (50 ml). After cooling to room temperature, a diluted ammonium hydroxide solution was slowly added dropwise till pH 7. The resulting precipitate was filtered off, washed with water and dried overnight at 100° C. to give the pure title compound (5.8 g, 98%). The obtained compound was used a such for further reactions without additional purification. M.p.>330° C.; elemental analysis calc. for C₇H₅ClN₄O (196.6): C 42.77H 2.56 N 28.50. Found: C 41.61H 2.74 N 28.76.

### EXAMPLE 281

### Synthesis of 2-acetamido-6-chloro-pyrido[3,2-d]pyrimidin-4(3H)-one

A suspension of 2-amino-6-chloro-pyrido[3,2-d]pyrimidin-4(3H)-one (6.2 g, 31.54 mmol) in acetic anhydride (620 ml) was refluxed with stirring for 4 hours. The hot mixture was filtered to remove insoluble material and the filtrate was evaporated to dryness. To the residue was added methanol (50 ml). The precipitate was filtered, washed with methanol and dried yielding the title compound (5.3 g, 70%) which was characterised as follows:

M.p. 317-319° C.;

UV (MeOH): 208 (4.13), 216 (sh 4.17), 280 (4.13), 310 (sh 3.44); and
Elemental analysis: calc. for C₉H₇ClN₄O₂ (238.6): C 45.30H 2.96 N 23.48. Found: C 45.61H 3.53 N 23.28.

### EXAMPLES 282 TO 289

### Synthesis of 2-acetamido-4-alkoxy-6-chloro-pyrido[3,2-d]pyrimidines

To a mixture of 2-acetamido-6-chloro-pyrido[3,2-d]pyrimidin-4(3H)-one (0.72 g, 3 mmol), triphenylphosphine (1.18 g, 4.5 mmol), and the appropriate alcohol (4.5 mmol) in dioxane (50 ml) was added diisopropyl azodicarboxylate (0.91 g, 0.87 ml, 4.5 mmol). The mixture was stirred at room temperature for 24-36 hr and then evaporated in vacuo. The residue was purified by silica gel flash chromatography. The compound was eluted with the following solvent systems: CH₂Cl₂ (500 ml), CH₂Cl₂/AcOEt 5:1 (600 ml), CH₂Cl₂/AcOEt 4:1 (500 ml), CH₂Cl₂/AcOEt 1:1 (300 ml), CH₂Cl₂/MeOH 100:5 (500 ml). Evaporation of the product fractions gave the desired 4-alkyloxy-2-amino-6-chloropyrido[3,2-d]pyrimidine in yields of 45-60%, depending on the alcohol used. Analytical samples were obtained by crystallization of the 2-amino-4-alkoxy-6-chloro-pyrido[3,2-d]pyrimidine from ethyl acetate, diethylether or methanol. Unreacted 2-acetamido-6-chloro-pyrido[3,2-d]pyrimidin-4(3H)-one (40 to 20%) was also isolated during chromatography. The following compounds were synthesized according to this general procedure:

### EXAMPLE 282

### 2-acetamido-4-ethoxy-6-chloro-pyrido[3,2-d]pyrimidine

From ethanol (210 mg, 4.5 mmol) to give the pure title compound (0.48 g, 60%) which was characterised as follows:

M.p. 233° C.;

UV (MeOH): 237 (4.58), 266 (4.15), 274 (4.14), 321 (3.73).
Calc. for C₁₁H₁₁ClN₄O₂ (266.7): C 49.54H 4.16 N 21.01. Found: C 49.01H 4.30 N 20.70.

### EXAMPLE 283

### 2-acetamido-4-n-propoxy-6-chloro-pyrido[3,2-d]pyrimidine

From n-propanol (270 mg, 4.5 mmol) to give the pure title compound (0.42 g, 50%) which was characterised as follows:

M.p. 191° C.;

UV (MeOH): 237 (4.58), 266 (4.15), 274 (4.14), 321 (3.73); and
Calc. for C₁₂H₁₃ClN₄O₂ (280.7): C 51.35H 4.67 N 19.96. Found: C 51.16H 4.69 N 19.94.

### EXAMPLE 284

### 2-acetamido-4-isopropoxy-6-chloro-pyrido[3,2-d]pyrimidine

From isopropanol (270 mg, 4.5 mmol) to give the pure title compound (0.479 g, 57%) which was characterised as follows:

M.p. 244° C.;

UV (MeOH): 237 (4.59), 266 (4.15), 274 (4.15), 321 (3.73);
Calc. for C₁₂H₁₃ClN₄O₂ (280.7): C 51.35H 4.67 N 19.96. Found: C 51.30H 4.71 N 20.05.

### EXAMPLE 285

### 2-acetamido-4-n-butoxy-6-chloro-pyrido[3,2-d]pyrimidine

From n-butanol (270 mg, 4.5 mmol) to give the pure title compound (0.504 g, 57%) which was characterised as follows:

M.p. 158-159° C.;

UV (MeOH): 237 (4.59), 266 (4.15), 274 (4.15), 321 (3.73); and
Calc. for C₁₃H₁₅ClN₄O₂ (294.7): C 52.98H 5.13 N 19.01. Found: C 52.11H 5.16 N 18.68.

### EXAMPLE 286

### 2-acetamido-4-isobutoxy-6-chloro-pyrido[3,2-d]pyrimidine

From isobutanol (333 mg, 4.5 mmol) to yield the pure title compound (0.46 g, 52%) which was characterised as follows:

M.p. 168° C.;

UV (MeOH): 237 (4.59), 266 (4.16), 274 (4.15), 321 (3.75);
Calc. for C₁₃H₁₅ClN₄O₂ (294.7): C 52.98H 5.13 N 19.01. Found: C 52.87H 5.16 N 19.07.

### EXAMPLE 287

### 2-acetamido-4-sec.butoxy-6-chloro-pyrido[3,2-d]pyrimidine

From sec-butanol (400 mg, 4.5 mmol) to yield the pure title compound (0.442 g, 50%) which was characterised as follows:

M.p. 143-144° C.;

UV (MeOH): 237 (4.56), 266 (4.13), 274 (4.18), 321 (3.71); and
Calc. for C₁₃H₁₅ClN₄O₂ (294.7): C 52.98H 5.13 N 19.01. Found: C 52.85H 5.13 N 18.92.

### EXAMPLE 288

### 2-acetamido-4-n-pentoxy-6-chloro-pyrido[3,2-d]pyrimidine

From n-pentanol (333 mg, 4.5 mmol) to yield the pure title compound (0.37 g, 40%) which was characterised as follows:

M.p. 174° C.;

UV (MeOH): 238 (4.60), 266 (4.13), 275 (4.13), 322 (3.72); and

Calc. for C₁₄H₁₇ClN₄O₂ (308.8): C 54.46H 5.55 N 18.15. Found: C 54.47H 5.66 N 18.14.

### EXAMPLE 289

### 2-acetamido-4-benzyloxy-6-chloro-pyrido[3,2-d]pyrimidine

From benzylalcohol (486 mg, 4.5 mmol) and stirring for 72 hours to give the pure title compound as a yellowish powder (240 mg, 24%) which was characterised as follows:

M.p. 199-200° C.;

UV (MeOH): 207 (4.40), 237 (4.56), 265 (4.15), 274 (4.13), 322 (3.74);
Calc. for C₁₆H₁₃ClN₄O₂ (328.8): C 58.46H 3.99 N 17.04. Found: C 58.56H 4.04 N 17.05.

### EXAMPLES 290 TO 312

### Synthesis of 2-amino-4-alkoxy- and 2-amino-4-benzyloxy-6-(fluorophenyl)pyrido[3,2-d]pyrimidines

To a degassed suspension of a 2-acetamido-4-alkoxy-6-chloro-pyrido[3,2-d]pyrimidine (0.5 mmol), 2-, 3-, or 4-fluorophenylboronic acid (80 mg, 0.57 mmol) and potassium carbonate (2-4 mmol) in a mixture of dioxane (7.3 ml) and water (1.6 ml) was added tetrakis(triphenylphosphine)palladium(0) (29 mg, 0.025 mmol). The mixture was refluxed (bath temperature 120° C.) for 24 hours. After cooling to room temperature dichloromethane (30 ml) was added and the mixture was washed with a brine solution. The organic layer was separated, dried over Na₂SO₄ and evaporated in vacuo. The resulting crude material was purified by silica gel flash chromatography. The compound was eluted with the following solvent systems: CH₂Cl₂ (100 ml), CH₂Cl₂/MeOH 100:1 (101 ml), 100:2 (102 ml), 100:3 (103 ml). Evaporation of the product fractions afforded 2-amino-4-O-substituted-6-(fluorophenyl)pyrido[3,2-d]pyrimidines as crystal solids in yields varying from 70-85%. In some cases the corresponding 2-acetamidoderivates were detected and also isolated as the faster-moving component. The analytical samples were prepared by recrystallization from ether or methanol. The following compounds were synthesized according to this general procedure:

### EXAMPLE 290

### 2-amino-4-ethoxy-6-(o-fluorophenyl)-pyrido[3,2-a]pyrimidine

Analogous to the general procedure with 2-fluorophenylboronic acid (80 mg, 0.57 mmol) to yield the pure title compound (0.657 g, 77%) which was characterised as follows:

M.p. 182° C.;

UV (MeOH): 231 (4.47), 284 (4.29), 348 (3.89); and
Calc. for C₁₅H₁₃FN₄O (284.3): C 63.37H 4.61 N 19.41. Found: C 62.70H 4.65 N 19.41

### EXAMPLE 291

### 2-amino-4-ethoxy-6-(m-fluorophenyl)-pyrido[3,2-d]-pyrimidine

Analogous to the general procedure with 3-fluorophenylboronic acid (80 mg, 0.57 mmol) to yield the pure title compound (0.69 g, 81%) which was characterised as follows:

M.p. 174° C.;

UV (MeOH): 234 (4.43), 292 (4.31), 352 (3.92); and
Calc. for C₁₅H₁₃FN₄O (284.3): C 63.37H 4.61 N 19.41. Found: C 62.51H 4.72 N 19.10

### EXAMPLE 292

### 2-amino-4-ethoxy-6-(p-fluorophenyl)pyrido[3,2-d]-pyrimidine

Analogous to the general procedure with 4-fluorophenyl boronic acid (80 mg, 0.57 mmol) to give the pure title compound (0.657 g, 77%) which was characterised as follows:

M.p. 188-189° C.;

UV (MeOH): 216 (4.48), 234 (4.44), 287 (4.34), 354 (3.89); and
Calc. for C₁₅H₁₃FN₄O (284.3): C 63.37H 4.61 N 19.41. Found: C 62.98H 4.63 N 19.67

### EXAMPLE 293

### 2-amino-4-n-propoxy-6-(o-fluorophenyl)-pyrido[3,2-d]pyrimidine

Analogous to the general procedure with 2-fluorophenyl boronic acid (80 mg, 0.57 mmol) to yield the pure title compound (0.698 g, 78%) which was characterised as follows:

M.p. 191° C.;

UV (MeOH): 231 (4.49), 284 (4.30), 348 (3.90);
Calc. for C₁₅H₁₃FN₄O (298.3): C 64.42H 5.07 N 18.78. Found: C 64.15H 5.00 N 18.76.

### EXAMPLE 294

### 2-amino-4-n-propoxy-6-(P-fluorophenyl)-pyrido[3,2-d]pyrimidine

Analogous to the general procedure with 4-fluorophenyl boronic acid (80 mg, 0.57 mmol) to yield the pure title compound (0.698 g, 78%) which was characterised as follows:

M.p. 185-186° C.;

UV (MeOH): 216 (4.50), 233 (4.46), 287 (4.35), 353 (3.90); and
Calc. for C₁₅H₁₃FN₄O (298.3): C 64.42H 5.07 N 18.78. Found: C 63.86H 5.37 N 18.46.

### EXAMPLE 295

### 2-amino-4-isopropoxy-6-(m-fluorophenyl)-pyrido[3,2-d]pyrimidine

Analogous to the general procedure with 3-fluorophenylboronic acid (80 mg, 0.57 mmol) to yield the pure title compound (0.698 g, 78%) which was characterised as follows:

M.p. 200-201° C.;

UV (MeOH): 236 (4.38), 292 (4.29), 352 (3.91),
Calc. for C₁₆H₁₅FN₄O (298.3): C 64.42H 5.07 N 18.78. Found: C 63.07H 5.08 N 18.06

### EXAMPLE 296

### 2-acetamido-4-isopropoxy-6-(p-fluorophenyl)-pyrido[3,2-d]pyrimidine

Analogous to the general procedure with 4-fluorophenyl boronic acid (80 mg, 0.57 mmol) and isolated from the first fraction on column chromatography to give the pure title compound (0.694 g, 68%) which was characterised as follows:

M.p. 196-197° C.;

UV (MeOH): 239 (4.39), 257 (4.24), 286 (4.30), 334 (3.99); and
Calc. for C₁₈H₁₇FN₄O₂ (340.4): C 63.52H 5.03 N 16.46. Found: C 62.65H 4.73 N 16.40

### EXAMPLE 297

### 2-amino-4-isopropoxy-6-(p-fluorophenyl)-pyrido[3,2-d]pyrimidine

Analogous to the general procedure with 4-fluorophenylboronic acid (80 mg, 0.57 mmol) and isolated a the second fraction of column chromatography to give the pure title compound (0.143 g, 16%) which was characterised as follows:

M.p. 191-192° C.;

UV (MeOH): 216 (4.50), 233 (4.46), 287 (4.35), 353 (3.90); and
Calc. for C₁₆H₁₅FN₄O (298.3): C 64.42H 5.07 N 18.78. Found: C 64.25H 5.16 N 18.68

### EXAMPLE 298

### 2-amino-4-n-butoxy-6-(o-fluorophenyl)-pyrido[3,2-d]pyrimidine

Analogous to the general procedure with 2-fluorophenyl boronic acid (80 mg, 0.57 mmol) to give the pure title compound (0.75 g, 80%) which was characterised as follows:

M.p. 147-148° C.;

UV (MeOH): 232 (4.42), 284 (4.28), 348 (3.88); and
Calc. for C₁₇H₁₇FN₄O (312.4): C 65.37H 5.49 N 17.94. Found: C 64.55H 5.56 N 17.62

### EXAMPLE 299

### 2-amino-4-n-butoxy-6-(m-fluorophenyl)-pyrido[3,2-d]pyrimidine

Analogous to the general procedure with 3-fluorophenyl boronic acid (80 mg, 0.57 mmol) to give the pure title compound (0.61 g, 65%) which was characterised as follows:

M.p. 160-161° C.;

UV (MeOH): 236 (4.38), 292 (4.29), 352 (3.91);
Calc. for C₁₇H₁₇FN₄O (312.4): C 65.37H 5.49 N 17.94. Found: C 64.84H 5.65 N 18.03

### EXAMPLE 300

### 2-acetamido-4-n-butoxy-6-(p-fluorophenyl)-pyrido[3,2-d]pyrimidine

Analogous to the general procedure with 4-fluorophenyl boronic acid (80 mg, 0.57 mmol) and isolated from the first fraction of column chromatography to give the pure title compound (0.16 g, 15%) which was characterised as follows:

M.p. 170° C.;

UV (MeOH): 225 (4.32), 239 (4.39), 257 (4.22), 288 (4.32), 334 (4.00);
Calc. for C₁₉H₁₉FN₄O₂ (312.4): C 64.40H 5.40 N 15.81. Found: C 63.73H 5.54 N 15.50

### EXAMPLE 301

### 2-amino-4-n-butoxy-6-(p-fluorophenyl)-pyrido[3,2-d]pyrimidine

Analogous to the general procedure with 4-fluorophenyl boronic acid (80 mg, 0.57 mmol) and isolated from the second fraction of column chromatography to give the pure title compound (0.73 g, 78%) which was characterised as follows:

M.p. 172-173° C.;

UV (MeOH): 218 (4.50), 234 (4.39), 288 (4.35), 352 (3.89);
Calc. for C₁₇H₁₇FN₄O (312.4): C 65.37H 5.49 N 17.94. Found: C 64.84H 5.65 N 18.03

### EXAMPLE 302

### 2-amino-4-isobutoxy-6-(o-fluorophenyl)-pyrido[3,2-d]pyrimidine

Analogous to the general procedure with 2-fluorophenyl boronic acid (80 mg, 0.57 mmol) to give the pure title compound (0.75 g, 78%) which was characterised as follows:

M.p. 165° C.;

UV (MeOH): 232 (4.46), 284 (4.32), 348 (3.93); and
Calc. for C₁₇H₁₇FN₄O (312.4): C 65.37H 5.49 N 17.94. Found: C 65.60H 5.75 N 18.04

### EXAMPLE 303

### 2-amino-4-isobutoxy-6-(m-fluorophenyl)-pyrido[3,2-d]pyrimidine

Analogous to the general procedure with 3-fluorophenyl boronic acid (80 mg, 0.57 mmol) to give the pure title compound (0.75 g, 78%) which was characterised as follows:

M.p. 185° C.;

UV (MeOH): 236 (4.39), 292 (4.31), 352 (3.93);
Calc. for C₁₇H₁₇FN₄O (312.4): C 65.37H 5.49 N 17.94. Found: C 65.59H 5.55 N 18.00

### EXAMPLE 304

### 2-amino-4-isobutoxy-6-(p-fluorophenyl)-pyrido[3,2-d]pyrimidine

Analogous to the general procedure with 4-fluorophenylboronic acid (80 mg, 0.57 mmol) to give the pure title compound (0.806 g, 86%) which was characterised as follows:

M.p. 196° C.;

UV (MeOH): 234 (4.40), 287 (4.34), 353 (3.89); and
Calc. for C₁₇H₁₇FN₄O (312.4): C 65.37H 5.49 N 17.94. Found: C 64.94H 5.42 N 17.90

### EXAMPLE 305

### 2-amino-4-sec.butoxy-6-(o-fluorophenyl)-pyrido[3,2-d]pyrimidine

Analogous to the general procedure with 2-fluorophenylboronic acid (80 mg, 0.57 mmol) to give the pure title compound (0.693 g, 74%) which was characterised as follows:

M.p. 159° C.;

UV (MeOH): 233 (4.42), 284 (4.27), 348 (3.89); and
Calc. for C₁₇H₁₇FN₄O (312.4): C 65.37H 5.49 N 17.94. Found: C 65.60H 5.42 N 17.70

### EXAMPLE 306

### 2-amino-4-sec.butoxy-6-(m-fluorophenyl)-pyrido[3,2-d]pyrimidine

Analogous to the general procedure with 3-fluorophenyl boronic acid (80 mg, 0.57 mmol) to give the pure title compound (0.646 g, 69%) which was characterised as follows:

M.p. 158-159° C.;

UV (MeOH): 237 (4.39), 292 (4.31), 352 (3.94); and
Calc. for C₁₇H₁₇FN₄O (312.4): C 65.37H 5.49 N 17.94. Found: C 64.58H 5.19 N 18.04

### EXAMPLE 307

### 2-amino-4-sec.butoxy-6-(p-fluorophenyl)-pyrido[3,2-d]pyrimidine

Analogous to the general procedure with 4-fluorophenyl boronic acid (80 mg, 0.57 mmol) to yield the pure title compound (0.645 g, 69%) which was characterised as follows:

M.p. 148° C.;

UV (MeOH): 234 (4.37), 287 (4.31), 354 (3.87); and
Calc. for C₁₇H₁₇FN₄O (312.4): C 65.37H 5.49 N 17.94. Found: C 65.28H 5.34 N 18.03

### EXAMPLE 308

### 2-amino-4-n-pentyloxy-6-(o-fluorophenyl)pyrido[3,2-d]-pyrimidine

Analogous to the general procedure with 2-fluorophenyl boronic acid (80 mg, 0.57 mmol) to give 0.803 g (82%) which was characterised as follows:

M.p. 136-137° C.;

UV (MeOH): 232 (4.43), 284 (4.28), 348 (3.89); and
Calc. for C₁₈H₁₉FN₄O (326.4): C 66.24H 5.87 N 17.17. Found: C 65.83H 5.62 N 17.14

### EXAMPLE 309

### 2-amino-4-n-pentyloxy-6-(m-fluorophenyl)-pyrido[3,2-c]pyrimidine

Analogous to the general procedure with 3-fluorophenyl boronic acid (80 mg, 0.57 mmol) to give the pure title compound (0.783 g, 80%) which was characterised as follows:

M.p. 142-143° C.;

UV (MeOH): 236 (4.39), 292 (4.30), 351 (3.92); and
Calc. for C₁₈H₁₉FN₄O (326.4): C 66.24H 5.87 N 17.17. Found: C 65.36H 5.72 N 16.52

### EXAMPLE 310

### 2-amino-4-benzyloxy-6-(o-fluorophenyl)-pyrido[3,2-d]pyrimidine

Analogous to the general procedure with 2-fluorophenyl boronic acid (80 mg, 0.57 mmol) to yield the pure title compound (0.748 g, 72%) which was characterised as follows:

M.p. 200-202° C.;

UV (MeOH): 208 (4.45), 232 (4.43), 285 (4.28), 350 (3.90); and
Calc. for C₂₀H₁₅FN₄O (346.4): C 69.36H 4.37 N 16.18. Found: C 69.16H 4.59 N 16.30

### EXAMPLE 311

### 2-amino-4-benzyloxy-6-(m-fluorophenyl)-pyrido[3,2-d]pyrimidine

Analogous to the general procedure with 3-fluorophenyl boronic acid (80 mg, 0.57 mmol) to give the pure title compound (0.717 g, 69%) which was characterised as follows:

M.p. 199-200° C.;

UV (MeOH): 208 (4.43), 235 (4.39), 292 (4.30), 352 (3.92); and
Calc. for C₂₀H₁₅FN₄O (346.4): C 69.36H 4.37 N 16.18. Found: C 69.07H 4.44 N 15.60

### EXAMPLE 312

### 2-amino-4-benzyloxy-6-(p-fluorophenyl)-pyrido[3,2-d]pyrimidine

Analogous to the general procedure with 4-fluorophenyl boronic acid (80 mg, 0.57 mmol) to give the pure title compound (0.81 g, 78%) which was characterised as follows:

M.p. 225° C.;

UV (MeOH): 210 (4.46), 233 (4.43), 287 (4.35), 354 (3.92); and
Calc. for C₂₀H₁₅FN₄O (346.4): C 69.36H 4.37 N 16.18. Found: C 69.16H 4.59 N 16.30.

### EXAMPLE 313

### Synthesis of 2-amino-4-(N-piperazin-1-yl)-6-(3,4-dimethoxyphenyl)-pyrido[3,2-d]pyrimidine

A mixture of 2-amino-6-(3,4-dimethoxy-phenyl)-pyrido[3,2-d]pyrimidin-4(3H)-one (722 mg, 2.42 mmol), 1,1,1,3,3,3-hexamethyldisilazane (2.6 ml, 12 mmol), piperazine (840 mg, 9.75 mmol), p-toluenesulphonic acid (60 mg, 0.32 mmol) and ammonium sulphate (47 mg, 0.36 mmol) in pyridine (12 ml) is refluxed for 2 days. Upon cooling down to room temperature, the reaction mixture is evaporated with silica gel. The residue is purified by silica gel flash chromatography, the mobile phase being a mixture of methanol and dichloromethane (in a ratio of 15:85, with 1% triethylamine), affording the pure title compound (439 mg). An impure fraction is purified further by preparative TLC on silica eluting with 20% MeOH and 1% Et₃N in CH₂Cl₂ to give another 140 mg of the title compound (combined yield: 579 mg, 65%).

MS (m/z): 367 ([M+H]⁺, 100)

### EXAMPLES 314 TO 318

### Synthesis of 2-amino-4-(N-acyl-piperazin-1-yl)-6-(3,4-dimethoxyphenyl)-pyrido[3,2-d]pyrimidines

To a suspension of the compound of example 313 (36 mg, 98 μmol) in CH₂Cl₂ (2 ml) and triethylamine (15 μl) is added an appropriate acid chloride (105 μmol). The reaction mixture was stirred at room temperature for 45 minutes. The solvents are evaporated in vacuo and the residue is purified by preparative TLC on silica gel. Elution with 5% MeOH in CH₂Cl₂ afforded the pure title compounds in yields varying from 55 to 90%, depending on the acid chloride used.

### EXAMPLE 314

### 2-amino-4-[N-(cyclohexanoyl)-piperazin-1-yl]-6-(3,4-dimethoxy-phenyl)-pyrido[3,2-d]pyrimidine

This compound was synthesized using cyclohexanecarbonyl chloride. MS (m/z): 477 ([M+H]⁺, 100)

### EXAMPLE 315

### 2-amino-4-[N-(propionyl)-piperazin-1-yl]-6-(3,4-dimethoxy-phenyl)-pyrido[3,2-d]pyrimidine

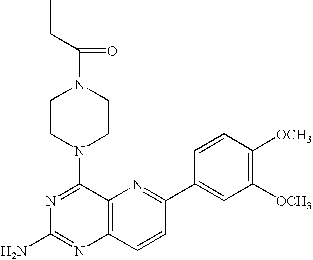

This compound was synthesized using propionyl chloride. MS (m/z): 423 ([M+H]⁺, 100)

### EXAMPLE 316

### 2-amino-4-[N-(hexanoyl)-piperazin-1-yl]-6-(3,4-dimethoxy-phenyl)-pyrido[3,2-d]pyrimidine

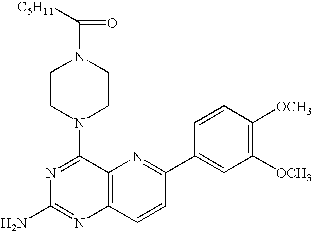

This compound was synthesized using hexanoyl chloride. MS (m/z): 465 ([M+H]⁺, 100).

### EXAMPLE 317

### 2-amino-4-[N-(methoxyacetyl)-piperazin-1-yl]-6-(3,4-dimethoxphenyl)-pyrido[3,2-d]pyrimidine (4AZA2613)

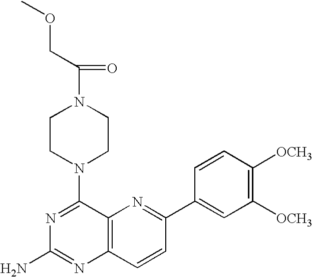

This compound was synthesized using methoxyacetyl chloride. MS (m/z): 439 ([M+H]⁺, 100).

### EXAMPLE 318

### 2-amino-4-[N-(methanesulfonyl)-piperazin-1-yl]-6-(3,4-dimethoxy-Phenyl)-pyrido[3,2-d]pyrimidine

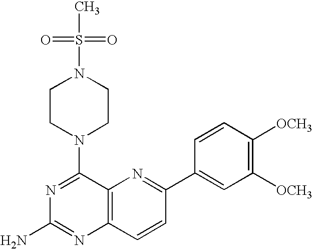

This compound was synthesized using methanesulfonyl chloride. MS (m/z): 445 ([M+H]⁺, 100)

### EXAMPLE 319

### Mixed Lymphocyte Reaction Assay

Pyrido[3,2-d]pyrimidine derivatives were first dissolved (10 mM) in dimethyl-sulfoxide (hereinafter referred as DMSO) and further diluted in culture medium before use for the following in vitro experiments. The commercially available culture medium consisted of RPMI-1640+10% foetal calf serum (FCS). Some pyrido[3,2-d]pyrimidine derivatives described herein were tested in the following mixed lymphocyte reaction (MLR) assay.

Peripheral blood mononuclear cells (hereinafter referred as PBMC) were isolated from heparinized peripheral blood by density gradient centrifugation over Lymphoprep (Nycomed, Maorstua, Norway). Allogeneic PBMC or Eppstein-Barr Virus-transformed human B cells [commercially available under the trade name RPM11788 (ATCC name CCL156)] which strongly express B7-1 and B7-2 antigens were used as stimulator cells after irradiation with 30 Gy. MLR was performed in triplicate wells. After 5 days incubation at 37° C., 1 μCi [³H]-thymidine was added to each cup. After a further 16 hours incubation, cells were harvested and counted in a β-counter. Inhibition of proliferation by a compound described in some of the present examples was counted while using the formula:

$$\% inhibition = \frac{( cpm + drugs ) - ( cpm cult . med )}{( cpm - drugs ) - ( OD cult . med )} \times 100$$

wherein cpm is the thymidine count per minute. The MLR assay is regarded by those skilled in the art as an in vitro analogue of the transplant rejection since it is based on the recognition of allogeneic major histocompatibility antigens on the stimulator leukocytes, by responding lymphocytes. The IC₅₀ value represents the lowest concentration of the pyrido[3,2-d]pyrimidine derivative (expressed in pmole/l) that resulted in a 50% suppression of the MLR. The following IC₅₀ values in the MLR test are mentioned in table 1 below.

### EXAMPLE 320

### TNF-α Assay

Peripheral blood mononuclear cells (herein referred as PBMC), in response to stimulation by lipopolysaccharide (hereinafter LPS), a gram-negative bacterial endotoxin, produce various chemokines, in particular human TNF-α. Inhibition of the activation of PBMC can therefore be measured by the level of suppression of the production of TNF-α by PBMC in response to stimulation by LPS. Inhibition measurement was performed as follows: PBMC were isolated from heparinized peripheral blood by density gradient centrifugation over Lymphoprep (commercially available from Nycomed, Norway). LPS was then added to the PMBC suspension in complete medium (10⁶ cells/ml) at a final concentration of 1 μg/ml. The pteridine derivative to be tested was added at different concentrations (0.1 μM, 1 μM and 10 μM) and the cells were incubated at 37° C. for 72 hours in 5% CO₂. The supernatants were collected, then TNF-α concentrations were measured with respectively an anti-TNF-α antibody in a sandwich ELISA (Duo Set ELISA human TNFα, commercially available from R&D Systems, United Kingdom). The colorimetric reading of the ELISA was measured by a Multiskan RC plate reader (commercially available from ThermoLabsystems, Finland) at 450 nm (reference wavelength: 690 nm). Data analysis was performed with Ascent software 2.6. (also from ThermoLabsystems, Finland): a standard curve (recombinant human TNFα) was drawn and the amount (μg/ml) of each sample on the standard curve was determined. The % suppression of human TNFα production by the pyrido[3,2-d]pyrimidine derivatives of the invention was calculated using the formula:

$$\% suppression = \frac{pg \text{/} ml in drugs - pg \text{/} ml in cult . med .}{( pg \text{/} ml in cult . med . + LPS ) - pg \text{/} ml cult . med .}$$

### EXAMPLE 321

### IL-1β Assay

Peripheral blood mononuclear cells (herein referred as PBMC), in response to stimulation by lipopolysaccharide (LPS), a gram-negative bacterial endotoxin, produce various chemokines, in particular human IL-1β. Inhibition of the activation of PBMC can therefore be measured by the level of suppression of the production of IL-1β by PBMC in response to stimulation by LPS.

Such inhibition measurement was performed as follows: PBMC were isolated from heparinized peripheral blood by density gradient centrifugation over Lymphoprep (commercially available from Nycomed, Norway). LPS was then added to the PMBC suspension in complete medium (10⁶ cells/ml) at a final concentration of 1 μg/ml. The pteridine derivative to be tested was added at different concentrations (0.1 μM, 1 μM and 10 μM) and the cells were incubated at 37° C. for 72 hours in 5% CO₂. The supernatants were collected, then IL-1α concentrations were measured with an anti-IL-1β antibody in a sandwich ELISA. The colorimetric reading of the ELISA was measured by a Multiskan RC plate reader (commercially available from ThermoLabsystems, Finland) at 450 nm (reference wavelength: 690 nm). Data analysis was performed with Ascent software 2.6. (also from ThermoLabsystems, Finland): a standard curve (recombinant human IL-1β) was drawn and the amount (μg/ml) of each sample on the standard curve was determined.

The % suppression of human IL-1β by the pyrido[3,2-d]pyrimidine derivatives of this invention was calculated using the formula:

$$\% suppression = \frac{pg \text{/} ml in drugs - pg \text{/} ml in cult . med .}{( pg \text{/} ml in cult . med . + LPS ) - pg \text{/} ml cult . med .}$$

### EXAMPLE 322

### Biological Activity of Pyrido[3,2-d]pyrimidine Derivatives

Some of the pyrido[3,2-d]pyrimidine derivatives being described in the previous examples have been tested for biological activities according to the methodologies of examples 169 to 171.

The detailed nomenclature of these pyrido[3,2-d]pyrimidine derivatives is shown in the following table 1, which also shows their IC₅₀ values (expressed in μM) in the MLR test of example 169 and in the TNF-α assay of example 170. IC₅₀ values found in the IL-1 assay of example 171 were:
6.9 μM for the derivative of example 32,
7.9 μM for the derivative of example 41, and
1.8 μM for the derivative of example 42.

TABLE 1

| Example | Derivative | MLR (μM) | TNF α (μM) |
|---|---|---|---|
| 5 | 4-[(2-phenoxyethyl)-piperazin-1-yl]-6-(3,4- | 0.1 | 0.65 |
| | dimethoxy-phenyl)-pyrido[3,2-d]pyrimidine | | |
| 8 | 4-(4-[3-methylphenyl)amino]carbonyl]piperazin- | 0.0094 | 0.07 |
| | 1-yl)-6-(3,4-dimethoxyphenyl)-pyrido[3,2- | | |
| | d]pyrimidine | | |
| 11 | 2-methyl-4-(4-[3- | 0.026 | 0.5 |
| | methylphenyl)amino]carbonyl]pipera-zin-1-yl)-6- | | |
| | (3,4-dimethoxyphenyl)-pyrido[3,2-d]pyrimi-dine | | |
| 14 | 2-chloro-4-(4-[3- | 0.066 | |
| | methylphenyl)amino]carbonyl]pipera-zin-1-yl)-6- | | |
| | (3,4-dimethoxyphenyl)-pyrido[3,2-d]pyrimi-dine | | |
| 15 | 2-dimethylamino-4-(4-[3- | 0.4 | 3.3 |
| | methylphenyl)amino]carbo-nyl]piperazin-1-yl)-6- | | |
| | (3,4-dimethoxyphenyl)-pyrido[3,2-d]pyrimidine | | |
| 16 | 2-[(N-hydroxyethyl)morpholino]-4-(4-[3- | 0.4 | |
| | methylphenyl) amino]carbonyl]piperazin-1-yl)-6- | | |
| | (3,4-dimethoxy-phenyl)-pyrido[3,2-d]pyrimidine | | |
| 17 | 2-(1-methyl-2-pyrrolidino-ethoxy)-4-(4-[3-methyl- | 2.7 | 5.2 |
| | phenyl)amino]carbonyl]piperazin-1-yl)-6-(3,4- | | |
| | dimethoxyphenyl)-pyrido[3,2-d]pyrimidine | | |
| 18 | 2-(2-phenoxyethoxy)-4-(4-[3- | 0.9 | |
| | methylphenyl)amino] carbonyl]piperazin-1-yl)-6- | | |
| | (3,4-dimethoxyphenyl)-pyrido[3,2-d]pyrimidine | | |
| 24 | 2-amino-4-isopropoxy-6-(3,4-dimethoxyphenyl)- | 0.066 | 0.5 |
| | pyrido[3,2-d]pyrimidine | | |
| 25 | 2-amino-4-phenoxyethoxy-6-(3,4- | 0.3 | 0.7 |
| | dimethoxyphenyl)-pyrido[3,2-d]pyrimidine | | |
| 26 | 2-amino-4-[(4-carboxylic ethyl ester)-piperidin-1- | 2.6 | 0.06 |
| | yl]-6-(3,4-dimethoxyphenyl)-pyrido[3,2- | | |
| | d]pyrimidine | | |
| 27 | 2-amino-4-(m-tolylamino)-6-(3,4- | 5.7 | 6.4 |
| | dimethoxyphenyl)-pyrido[3,2-d]pyrimidine | | |
| 28 | 2-amino-4-[3,4-(methylenedioxy)anilino]-6-(3,4- | 0.7 | 0.8 |
| | dimethoxyphenyl)-pyrido[3,2-d]pyrimidine | | |
| 29 | 2-amino-4-(m-bromophenylamino)-6-(3,4- | 0.7 | 0.8 |
| | dimethoxy-phenyl)-pyrido[3,2-d]pyrimidine | | |
| 30 | 2-amino-4-(2-chloro-5-methoxy-anilino)-6-(3,4- | 10 | |
| | dimethoxyphenyl)-pyrido[3,2-d]pyrimidine | | |
| 31 | 2-amino-4-(4-methylpiperazin-1-yl)-6-(3,4- | 0.9 | 0.8 |
| | dimethoxy-phenyl)-pyrido[3,2-d]pyrimidine | | |
| 32 | 2-amino-4-(thien-2-ylmethyl)amino-6-(3,4- | 0.8 | 0.8 |
| | dimethoxy-phenyl)-pyrido[3,2-d]pyrimidine | | |
| 33 | 2-amino-4-(2-N-morpholinylethyl)amino-6-(3,4- | 1.9 | 0.7 |
| | dimethoxyphenyl)-pyrido[3,2-d]pyrimidine | | |
| 34 | 2-amino-4-(2,2-dimethoxyethyl)amino-6-(3,4- | 0.8 | 0.6 |
| | dimethoxyphenyl)-pyrido[3,2-d]pyrimidine | | |
| 35 | 2-amino-4-(pyridin-2-yl-methyl)amino-6-(3,4- | 0.8 | 0.7 |
| | dimethoxyphenyl)-pyrido[3,2-d]pyrimidine | | |
| 36 | 2-amino-4-(4-aminocyclohexylamino)-6-(3,4- | 6.5 | 2.9 |
| | dimethoxyphenyl)-pyrido[3,2-d]pyrimidine | | |
| 41 | 2-amino-4-morpholino-6-(3,4-dimethoxyphenyl)- | 0.037 | 0.03 |
| | pyrido[3,2-d]pyrimidine | | |
| 42 | 2-amino-4-(4-[3- | 0.000064 | 0.06 |
| | methylphenyl)amino]carbonyl]pipera-zin-1-yl)-6- | | |
| | (3,4-dimethoxyphenyl)-pyrido[3,2-d]pyrimi-dine | | |
| 43 | 2-amino-4-(4-fluorophenyl-piperazin-1-yl)-6-(3,4- | 0.3 | 0.09 |
| | dimethoxyphenyl)-pyrido[3,2-d]pyrimidine | | |
| 44 | 2-amino-4-(4-methylphenyl-piperazin-1-yl)-6- | 0.16 | 0.3 |
| | (3,4-dimethoxyphenyl)-pyrido[3,2-d]pyrimidine | | |
| 45 | 2-amino-4-(phenoxyethyl-piperazin-1-yl)-6-(3,4- | 0.28 | 0.8 |
| | dimethoxyphenyl)-pyrido[3,2-d]pyrimidine | | |
| 46 | 2-amino-4-(3-chlorophenyl-piperazin-1-yl)-6- | 0.8 | 0.5 |
| | (3,4-dimethoxyphenyl)-pyrido[3,2-d]pyrimidine | | |
| 47 | 2-amino-4-(2-pyridyl-piperazin-1-yl)-6-(3,4- | 0.3 | 0.05 |
| | dimethoxyphenyl)-pyrido[3,2-d]pyrimidine | | |
| 48 | 2-amino-4-[2-(piperazin-1-yl)-acetic acid N-(2- | 0.06 | 0.06 |
| | thiazolyl)-amide]-6-(3,4-dimethoxyphenyl)- | | |
| | pyrido[3,2-d]pyrimidine | | |
| 49 | 2-amino-4-(N-acetyl-piperazin-1-yl)-6-(3,4- | 0.07 | 0.04 |
| | dimethoxy-phenyl)-pyrido[3,2-d]pyrimidine | | |
| 50 | 2-amino-4-(1-piperonyl-piperazin-1-yl)-6-(3,4- | 7.4 | 8.5 |
| | dimethoxyphenyl)-pyrido[3,2-d]pyrimidine | | |
| 51 | 2-amino-4-[1-(2-furoyl)-piperazin-1-yl]-6-(3,4- | 0.03 | 0.03 |
| | dimethoxy-phenyl)-pyrido[3,2-d]pyrimidine | | |
| 52 | 2-amino-4-(1-benzylpiperazin-1-yl)-6-(3,4- | 0.8 | 0.46 |
| | dimethoxy-phenyl)-pyrido[3,2-d]pyrimidine | | |
| 54 | 2-amino-4(N-3-thienyl-carbamoyl-piperazin-1- | 0.002 | 0.05 |
| | yl)-6-(3,4-dimethoxyphenyl)-pyrido[3,2- | | |
| | d]pyrimidine | | |
| 55 | 2-amino-4(N-2,6-dichloropyridinyl-carbamoyl- | 0.3 | 0.4 |
| | pipera-zin-1-yl)-6-(3,4-dimethoxyphenyl)- | | |
| | pyrido[3,2-d]pyrimi-dine | | |
| 56 | 2-amino-4(N-4-fluorophenyl-carbamoyl- | 0.003 | 0.07 |
| | piperazin-1-yl)-6-(3,4-dimethoxy-phenyl)- | | |
| | pyrido[3,2-d]pyrimidine | | |
| 57 | 2-amino-4(N-3-chloro-4-fluorophenyl-carbamoyl- | 0.004 | 0.3 |
| | piperazin-1-yl)-6-(3,4-dimethoxyphenyl)- | | |
| | pyrido[3,2-d]pyrimidine | | |
| 58 | 2-amino-4(N-3-chloro-phenyl-carbamoyl- | 0.0004 | 0.26 |
| | piperazin-1-yl)-6-(3,4-dimethoxyphenyl)- | | |
| | pyrido[3,2-d]pyrimidine | | |
| 59 | 2-amino-4[(N-4-chloro-phenoxy-acetyl)- | 0.016 | |
| | piperazin-1-yl]-6-(3,4-dimethoxyphenyl)- | | |
| | pyrido[3,2-d]pyrimidine | | |
| 60 | 2-amino-4[(N-phenoxy-acetyl)-piperazin-1-yl]-6- | 0.053 | |
| | (3,4-dimethoxyphenyl)-pyrido[3,2-d]pyrimidine | | |
| 70 | 4-[(N-3-chlorophenylcarbamoyl)-piperazin-1-yl]- | 3.4 | |
| | 6-chloro-pyrido[3,2-d]pyrimidine | | |
| 79 | 4-[(N-3-chlorophenylcarbamoyl)-piperazin-1-yl]- | 0.3 | |
| | 6-(3-chloro-4-methoxyphenyl)-pyrido[3,2- | | |
| | d]pyrimidine | | |
| 80 | 4-[(N-3-chlorophenylcarbamoyl)-piperazin-1-yl]- | 0.3 | |
| | 6-(1,4-benzodioxan-6-yl)-pyrido[3,2-d]pyrimidine | | |
| 81 | 4-[(N-3-chlorophenylcarbamoyl)-piperazin-1-yl]- | 0.5 | |
| | 6-(3,4-dimethylphenyl)-pyrido[3,2-d]pyrimidine | | |
| 82 | 4-[(N-3-chlorophenylcarbamoyl)-piperazin-1-yl]- | 0.3 | |
| | 6-(3,4-methylenedioxy)phenyl-pyrido[3,2- | | |
| | d]pyrimidine | | |
| 83 | 4-[(N-3-chlorophenylcarbamoyl)-piperazin-1-yl]- | 0.5 | |
| | 6-(3-chloro-4-ethoxyphenyl)-pyrido[3,2- | | |
| | d]pyrimidine | | |
| 84 | 4-[(N-3-chlorophenylcarbamoyl)-piperazin-1-yl]- | 0.7 | |
| | 6-(3,4-dichlorophenyl)-pyrido[3,2-d]pyrimidine | | |
| 88 | 2-morpholino-4-[(N-3-methyl-phenylcarbamoyl)- | 0.4 | |
| | piperazin-1-yl]-6-(3,4-dimethoxyphenyl)- | | |
| | pyrido[3,2-d] pyrimidine | | |
| 89 | 2-butoxy-4-[(N-3-methyl-phenylcarbamoyl)- | 2 | |
| | piperazin-1-yl]-6-(3,4-dimethoxyphenyl)- | | |
| | pyrido[3,2-d]pyrimidine | | |
| 90 | 2-methoxy-4-[(N-3-methyl-phenylcarbamoyl)- | 0.14 | 0.5 |
| | pipera-zin-1-yl]-6-(3,4-dimethoxyphenyl)- | | |
| | pyrido[3,2-d]pyrimi-dine | | |
| 91 | 2-(p-tolylamino-4-[(N-3-methyl- | 0.8 | |
| | phenylcarbamoyl)-piperazin-1-yl]-6-(3,4- | | |
| | dimethoxyphenyl)-pyrido[3,2-d] pyrimidine | | |
| 92 | 2-(3-chloro-4-fluoroanilino)-4-[(N-3-methyl- | 1.2 | |
| | phenyl-carbamoyl)-piperazin-1-yl]-6-(3,4- | | |
| | dimethoxyphenyl)-pyrido[3,2-d]pyrimidine | | |
| 93 | 2,4-diamino-6-(4-hydroxy-3-methoxyphenyl)- | 5.1 | |
| | pyrido[3,2-d] pyrimidine | | |
| 96 | 2-amino-4-(N-morpholino)-6-(4-hydroxy-3- | 0.8 | 0.6 |
| | methoxy)-pyrido[3,2-d]pyrimidine | | |
| 97 | 2-amino-4-(N-morpholino)-6-(4-ethoxy-3- | 0.5 | 0.3 |
| | methoxyphenyl)-pyrido[3,2-d]pyrimidine | | |
| 98 | 2-amino-4-(N-morpholino)-6-(4-cyclopentyloxy- | 7.6 | |
| | 3-methoxyphenyl)-pyrido[3,2-d]pyrimidine | | |
| 99 | 2-amino-4-(N-morpholino)-6-(4-isopropoxy-3- | 0.7 | 1 |
| | methoxy-phenyl)-pyrido[3,2-d]pyrimidine | | |
| 100 | 2-amino-4-(N-piperazin-1-yl)-6-(3-methoxy-4- | 3.3 | 2.4 |
| | hydroxy-phenyl)-pyrido[3,2-d]pyrimidine | | |
| 101 | 2-amino-4-[(N-4-fluoro-phenyl-carbamoyl)- | 0.5 | 0.6 |
| | piperazin-1-yl]-6-(4-hydroxy-3-methoxy-phenyl)- | | |
| | pyrido[3,2-d]pyri-midine | | |
| 102 | 2-amino-4-[(N-4-fluoro-phenyl-carbamoyl)- | 0.04 | 0.6 |
| | piperazin-1-yl]-6-(4-ethoxy-3-methoxyphenyl)- | | |
| | pyrido[3,2-d]pyrimi-dine | | |
| 103 | 2-amino-4-[(N-4-fluoro-phenyl-carbamoyl)- | 0.07 | 6.4 |
| | piperazin-1-yl]-6-(4-isopropoxy-3- | | |
| | methoxyphenyl)-pyrido[3,2-d] pyrimidine | | |
| 104 | 2-amino-4-[(N-3-methyl-phenyl-carbamoyl)- | 0.4 | 0.3 |
| | piperazin-1-yl]-6-(4-hydroxy-3-methoxyphenyl)- | | |
| | pyrido[3,2-d] pyrimidine | | |
| 105 | 4-(4-methyl-phenyl-piperazin-1-yl)-6-(3,4- | 0.8 | 0.09 |
| | dimethoxy-phenyl)-pyrido[3,2-d]pyrimidine | | |
| 107 | 4-(N-piperazin-1-yl)-6-(3,4-dimethoxyphenyl)- | 3.8 | 2.7 |
| | pyrido [3,2-d]pyrimidine | | |
| 108 | 4-(N-3-chloro-4-fluoro-phenylcarbamoyl- | 0.06 | 0.2 |
| | piperazin-1-yl)-6-(3,4-dimethoxyphenyl)- | | |
| | pyrido[3,2-d]pyrimidine | | |
| 109 | 4-[N-2-thienyl-carbamoyl)-piperazin-1-yl]-6-(3,4- | 0.06 | 0.13 |
| | dimethoxyphenyl)-pyrido[3,2-d]pyrimidine | | |
| 110 | 4-[N-2,6-dichloropyridyl-carbamoyl)-piperazin-1- | 10 | 6 |
| | yl]-6-(3,4-dimethoxyphenyl)-pyrido[3,2- | | |
| | d]pyrimidine | | |
| 111 | 4-[N-4-fluorophenyl-carbamoyl)-piperazin-1-yl]- | 0.03 | 0.03 |
| | 6-(3,4-dimethoxyphenyl)-pyrido[3,2-d]pyrimidine | | |
| 112 | 4-[N-3-chlorophenyl-carbamoyl)-piperazin-1-yl]- | 0.04 | 0.07 |
| | 6-(3,4-dimethoxyphenyl)-pyrido[3,2-d]pyrimidine | | |
| 113 | 4-[(N-4-chlorophenoxy-acetyl)-piperazin-1-yl]-6- | 0.04 | 0.3 |
| | (3,4-dimethoxyphenyl)-pyrido[3,2-d]pyrimidine | | |
| 116 | 4-(piperazin-1-yl)-6-(3-methyl-4-methoxyphenyl)- | 5.1 | 8.1 |
| | pyrido [3,2-d]pyrimidine | | |
| 117 | 4-[(N-3-chloro-phenylcarbamoyl)-piperazin-1-yl]- | 0.5 | 9 |
| | 6-(3-methyl-4-methoxyphenyl)-pyrido[3,2- | | |
| | d]pyrimidine | | |
| 118 | 4-[(N-4-chloro-phenylcarbamoyl)-piperazin-1-yl]- | 0.6 | |
| | 6-(3-methyl-4-methoxyphenyl)-pyrido[3,2- | | |
| | d]pyrimidine | | |
| 119 | 4-[(N-3-chloro-phenylcarbamoyl)-piperazin-1-yl]- | 0.6 | |
| | 6-(3-methoxy-4-hydroxyphenyl)-pyrido[3,2- | | |
| | d]pyrimidine | | |
| 120 | 4-[(N-3-chloro-phenylcarbamoyl)-piperazin-1-yl]- | 0.07 | 0.8 |
| | 6-(3-methoxy-4-ethoxyphenyl)-pyrido[3,2- | | |
| | d]pyrimidine | | |
| 121 | 4-[(N-3-chloro-phenylcarbamoyl)-piperazin-1-yl]- | 0.3 | |
| | 6-(3-methoxy-4-isopropoxy-phenyl-pyrido[3,2- | | |
| | d]pyrimidine | | |
| 124 | 4-morpholino-6-(4-chlorophenyl)-pyrido[3,2- | 7.2 | 10 |
| | d]pyrimi-dine | | |
| 145 | 2-acetamido-4-(1,2,4-triazolyl)-6-(3,4-dichloro- | 5.3 | |
| | phenyl)pyrido-[3,2-d] pyrimidine | | |
| 156 | 2-amino-4[(N-3-chloro-phenyl-carbamoyl)- | 0.1 | 5.5 |
| | piperazin-1-yl]-6-(3-methyl-4-methoxy- | | |
| | phenyl)pyrido-[3,2-d]pyrimi-dine | | |
| 157 | 2-amino-4[(N-3-chloro-phenyl-carbamoyl)- | 0.068 | 1.6 |
| | piperazin-1-yl]-6-(3-chloro-4-methoxy- | | |
| | phenyl)pyrido-[3,2-d]pyrimi-dine | | |
| 159 | 2-amino-4[(N-3-chloro-phenyl-carbamoyl)- | 0.07 | 4.8 |
| | piperazin-1-yl]-6-(3-fluoro-4-ethoxy- | | |
| | phenyl)pyrido-[3,2-d]pyrimidine | | |
| 161 | 2-amino-4[(N-3-chloro-phenyl-carbamoyl)- | 0.06 | |
| | piperazin-1-yl]-6-(3,4- | | |
| | methylenedioxy)phenyl)pyrido-[3,2-d]pyrimi-dine | | |
| 162 | 2-amino-4[(N-3-chloro-phenyl-carbamoyl)- | 0.1 | 10 |
| | piperazin-1-yl]-6-(1,4-benzodioxane- | | |
| | phenyl)pyrido-[3,2-d]pyrimi-dine | | |
| 163 | 2-amino-4-morpholino-6-(3-methyl-4- | 3.6 | 6.5 |
| | methoxyphenyl) pyrido[3,2-d]pyrimidine | | |
| 164 | 2-amino-4-(morpholino)-6-(3-chloro-4- | 0.6 | 1.5 |
| | methoxyphenyl)pyrido[3,2-d]pyrimidine | | |
| 165 | 2-amino-4-morpholino-6-(1,4-benzodioxane- | 0.6 | 0.9 |
| | phenyl) pyrido[3,2-d]pyrimidine | | |
| 166 | 2-amino-4-morpholino-6-(3-fluoro-4- | 0.7 | 0.8 |
| | ethoxyphenyl) pyrido[3,2-d]pyrimidine | | |
| 168 | 2-amino-4-morpholino-piperazin-1-yl]-6-(3,4- | 2.4 | |
| | (methylenedioxy)phenyl)pyrido [3,2-d]pyrimidine | | |
| 189 | 2-amino-4-[(N-4-chloro-benzylcarbamoyl)- | 0.5 | |
| | piperazin-1-yl]-6-(4-fluorophenyl)-pyrido[3,2- | | |
| | d]pyrimidine | | |
| 190 | 2-amino-4-[N-acetyl-piperazin-1-yl]-6-(3,4- | 0.8 | 4.8 |
| | methylenedioxyphenyl)-pyrido[3,2-d]pyrimidine | | |
| 191 | 2-amino-4-[2-(piperazin-1-yl acetic acid N-(2- | 0.4 | 3.5 |
| | thiazolyl)-amide)]-6-3,4-methylenedioxyphenyl)- | | |
| | pyrido[3,2-d]pyrimidine | | |
| 192 | 2-amino-4-[N-(2-furoyl)-piperazin-1-yl]-6-(3,4- | 8.1 | |
| | methylenedioxyphenyl)-pyrido[3,2-d]pyrimidine | | |
| 193 | 2-amino-4-[N-(4-chlorophenoxy-acetyl)- | 0.04 | 5.8 |
| | piperazin-1-yl]-6-(3,4-methylenedioxyphenyl)- | | |
| | pyrido[3,2-d]pyrimidine | | |
| 196 | 2-amino-4-[N-(3-methyl-phenyl-carbamoyl)- | 0.03 | 5.3 |
| | piperazin-1-yl]-6-(3,4-methylenedioxyphenyl)- | | |
| | pyrido[3,2-d]pyrimidine | | |
| 199 | 2-amino-4-[N-acetyl-piperazin-1-yl]-6-(1,4- | 4.8 | 2.9 |
| | benzodioxane)-pyrido[3,2-d]pyrimidine | | |
| 201 | 2-amino-4-[2-(piperazin-1-yl acetic acid N-(2- | 0.8 | 3.4 |
| | thiazolyl)-amide]-6-(1,4-benzodioxane)- | | |
| | pyrido[3,2-d]pyrimidine | | |
| 203 | 2-amino-4-[N-(2-furoyl)-piperazin-1-yl]-6-(1,4- | 5.7 | |
| | benzodioxane)-pyrido[3,2-d]pyrimidine | | |
| 204 | 2-amino-4-[N-(4-fluoro-phenyl)-piperazin-1-yl]-6- | 6.4 | |
| | (4-fluorophenyl)-pyrido[3,2-d]pyrimidine | | |
| 205 | 2-amino-4-[N-(phenoxy-ethyl)-piperazin-1-yl)]-6- | 5.5 | |
| | (4-fluorophenyl)-pyrido[3,2-d]pyrimidine | | |
| 207 | 2-amino-4-[(N-4-chloro-phenoxy-acetyl)- | 0.03 | |
| | piperazin-1-yl]-6-(4-fluorophenyl)-pyrido[3,2- | | |
| | d]pyrimidine | | |
| 210 | 2-amino-4-morpholino-6-(2-bromo-phenyl)- | 1.8 | 7.2 |
| | pyrido[3,2-d]pyrimidine | | |
| 211 | 4-[N-(3-chloro-phenylcarbamoyl)-piperazin-1-yl]- | 0.4 | |
| | 6-(3-methoxy-4-cyclopropylmethoxy-phenyl)- | | |
| | pyrido[3,2-d]pyrimidine | | |
| 212 | 4-[N-(3-chloro-phenylcarbamoyl)-piperazin-1-yl]- | 0.04 | 0.9 |
| | 6-(3-hydroxy-4-methoxy-phenyl)-pyrido[3,2- | | |
| | d]pyrimidine | | |
| 213 | 4-[N-(3-chloro-phenylcarbamoyl)-piperazin-1-yl]- | 0.057 | 0.06 |
| | 6-(3-ethoxy-4-methoxy-phenyl)-pyrido[3,2- | | |
| | d]pyrimidine | | |
| 214 | 4-[N-(3-chloro-phenylcarbamoyl)-piperazin-1-yl]- | 0.05 | 0.3 |
| | 6-(3-isopropoxy-4-methoxy-phenyl)-pyrido[3,2- | | |
| | d]pyrimidine | | |
| 215 | 4-[N-(3-chloro-phenylcarbamoyl)-piperazin-1-yl]- | 0.2 | 0.8 |
| | 6-(3-cyclopropylmethoxy-4-methoxy-phenyl)- | | |
| | pyrido[3,2-d]pyrimidine | | |
| 219 | 2-amino-4-[(S)-3-(amino)pyrrolidine]-6-(3,4- | 5.5 | 1.9 |
| | dimethoxyphenyl)-pyrido[3,2-d]pyrimidine | | |
| 220 | 2-amino-4-[3-(S)-4-chloro-phenoxy-acetyl- | 4.3 | 1.8 |
| | amino)-pyrrolidin-1-yl]-6-(3,4-dimethoxyphenyl)- | | |
| | pyrido[3,2-d]pyrimidine | | |
| 221 | 2-amino-4-[3-(S)-3-methyl phenyl carbamoyl | 1.3 | |
| | pyrrolidin-1-yl]-6-(3,4-dimethoxyphenyl)- | | |
| | pyrido[3,2-d]pyrimidine | | |
| 223 | 2-amino-4-thiomethyl-6-(3,4-dimethoxyphenyl)- | 0.9 | 0.8 |
| | pyrido[3,2-d]pyrimidine | | |
| 230 | of 2-amino-6-chloro-4-morpholino-pyrido[3,2- | >10 | |
| | d]pyrimidine | | |
| 232 | 2-amino-4-morpholino-6-(2-furan)-pyrido[3,2- | 5.3 | 5.3 |
| | d]pyrimidine | | |
| 233 | 2-amino-4-morpholino-6-(3-thiophene)- | 4.4 | 3.7 |
| | pyrido[3,2-d]pyrimidine | | |
| 234 | 2-amino-4-morpholino-6-(4-pyridinyl)-pyrido[3,2- | 0.7 | 0.5 |
| | d]pyrimidine | | |
| 235 | 2-amino-4-morpholino-6-(5-methyl-2-thiophene)- | 3.4 | 1.8 |
| | pyrido[3,2-d]pyrimidine | | |
| 236 | 2-amino-4-morpholino-6-(6-methoxy-2-pyridinyl)- | 5.3 | |
| | pyrido[3,2-d]pyrimidine | | |
| 237 | 2-amino-4-morpholino-6-(5-indole)-pyrido[3,2- | 0.8 | 2.6 |
| | d]pyrimidine | | |
| 238 | 2-amino-4-morpholino-6-(2-thiophene)- | 0.8 | 2.8 |
| | pyrido[3,2-d]pyrimidine | | |
| 239 | 2-amino-4-morpholino-6-(4-methyl-2-thiophene)- | 4.6 | 4.9 |
| | pyrido[3,2-d]pyrimidine | | |
| 240 | 2-amino-4-morpholino-6-(3-pyridinyl)-pyrido[3,2- | 1.3 | 0.7 |
| | d]pyrimidine | | |
| 241 | 2-amino-4-morpholino-6-(5-chloro-2-thiophene)- | 2.2 | 5.4 |
| | pyrido[3,2-d]pyrimidine | | |
| 242 | 2-amino-4-morpholino-6-(3-chloro-4- | 2.6 | |
| | fluorophenyl)-pyrido[3,2-d]pyrimidine | | |
| 243 | 2-amino-4-morpholino-6-(3,4-difluorophenyl)- | 2.3 | 6.0 |
| | pyrido[3,2-d]pyrimidine | | |
| 244 | 2-amino-4-morpholino-6-(4-fluoro-3- | 3.8 | 7.2 |
| | methylphenyl)-pyrido[3,2-d]pyrimidine | | |
| 245 | 2-amino-4-morpholino-6-(4-fluorophenyl)- | 2.3 | |
| | pyrido[3,2-d]pyrimidine | | |
| 246 | 2-amino-4-morpholino-6-[4-(3,5- | 5.5 | 1.6 |
| | dimethylisoxazole)]-pyrido[3,2-d]pyrimidine | | |
| 250 | 2-amino-4-[(N-3-methylphenylcarbamoyl)- | 0.0085 | 0.6 |
| | homopiperazin-1-yl]-6-(3,4-dimethoxyphenyl)- | | |
| | pyrido[3,2-d]pyrimidine | | |
| 255 | 2-amino-4-[(1-Boc-piperidin-4-yl)amino]-6-(3,4- | 0.06 | 0.04 |
| | dimethoxyphenyl)-pyrido[3,2-d]pyrimidine | | |
| 256 | 2,4-diamino-6-(3,4-dimethoxyphenyl)-pyrido[3,2- | 0.6 | |
| | d]pyrimidine | | |
| 257 | 2-amino-4-[(1-Boc-piperidin-3-yl)amino]-6-(3,4- | 0.3 | 0.6 |
| | dimethoxyphenyl)-pyrido[3,2-d]pyrimidine | | |
| 258 | 2-amino-4-[(1-Cbz-piperidin-3-yl)amino]-6-(3,4- | 0.9 | 0.3 |
| | dimethoxyphenyl)-pyrido[3,2-d]pyrimidine | | |
| 260 | 2-amino-4-[3-(R)-(3-methylphenylcarbamoyl)- | 2.1 | |
| | pyrrolidin-1-yl]-6-(3,4-dimethoxyphenyl)- | | |
| | pyrido[3,2-d]pyrimidine | | |
| 261 | 2-amino-4-[(3-methylphenylcarbamoyl)- | 0.08 | 0.8 |
| | ethylenediamine-1-N-yl]-6-(3,4- | | |
| | dimethoxyphenyl)-pyrido[3,2-d]pyrimidine | | |
| 262 | 2-amino-4-[(3-methylphenylcarbamoyl)-3- | 0.5 | 6.9 |
| | aminopropane-amino-1-N-yl]-6-(3,4- | | |
| | dimethoxyphenyl)-pyrido[3,2-d]pyrimidine | | |
| 263 | 2-amino-4-[1-(3- | 0.07 | 0.5 |
| | methylphenylcarbamoyl)piperidin-4-yl)amino]-6- | | |
| | (3,4-dimethoxyphenyl)-pyrido[3,2-d]pyrimidine | | |
| 264 | 2-amino-4-[(3-methylphenylcarbamoylpiperidin- | 0.5 | 1.8 |
| | 3-yl)amino)-6-(3,4-dimethoxyphenyl]-pyrido[3,2- | | |
| | d]pyrimidine | | |
| 265 | 2-amino-4-[2-(4-chlorophenoxy-acetyl- | 0.3 | 3.9 |
| | ethylenediamine-1-N-yl]-6-(3,4- | | |
| | dimethoxyphenyl)-pyrido[3,2-d]pyrimidine | | |
| 266 | 2-amino-4-[3-N-(4-chlorophenoxy-acetyl)-3- | 0.7 | 0.9 |
| | amino-propane-amine-1-N-yl]-6-(3,4- | | |
| | dimethoxyphenyl)-pyrido[3,2-d]pyrimidine | | |
| 267 | 2-amino-4-[(3-(R)-(4-chlorophenoxyacetyl- | 0.7 | 2.5 |
| | amino)-pyrrolidin-1-yl]-6-(3,4-dimethoxyphenyl)- | | |
| | pyrido[3,2-d]pyrimidine | | |
| 268 | 2-amino-4-[(3-carboxylic acid isobutylamide)- | 0.5 | 0.7 |
| | piperidin-1-yl]-6-(3,4-dimethoxyphenyl)- | | |
| | pyrido[3,2-d]pyrimidine | | |
| 269 | 2-amino-4-(4-chlorophenyl-4-hydroxypiperidin-1- | 3.1 | |
| | yl)-6-(3,4-dimethoxyphenyl)-pyrido[3,2- | | |
| | d]pyrimidine | | |
| 270 | 2-amino-4-[4-(N-2-phenylethylacetamid-2- | | 0.3 |
| | yl)piperazin-1-yl]-6-(3,4-dimethoxyphenyl)- | | |
| | pyrido[3,2-d]pyrimidine | | |
| 271 | 2-amino-4-[2-(4-benzylpiperazin-1-yl)-2-oxo- | 0.7 | 0.5 |
| | ethane-amino]-6-(3,4-dimethoxyphenyl)- | | |
| | pyrido[3,2-d]pyrimidine | | |
| 272 | 2-amino-4-[3-(4-acetylpiperazin-1-yl)-propan-3- | | 0.7 |
| | one-1-yl-amino]-6-(3,4-dimethoxyphenyl)- | | |
| | pyrido[3,2-d]pyrimidine | | |
| 273 | 2-amino-4-(N-pyrrolidinyl-acetamid-2-yl- | | 0.08 |
| | piperazin-1-yl)-6-(3,4- | | |
| | dimethoxyphenyl)pyrido[3,2-d]pyrimidine | | |
| 274 | 2-amino-4-(N-pyridinylacetamid-2-yl-piperazin-1- | | 0.08 |
| | yl)-6-(3,4-dimethoxyphenyl)-pyrido[3,2- | | |
| | d]pyrimidine | | |
| 275 | 2-amino-4-[N-(piperazino)-acetyl-morpholino]-6- | | 0.097 |
| | (3,4-dimethoxyphenyl)-pyrido[3,2-d]pyrimidine | | |
| 276 | 2-amino-4-[2-amino-1-(4-methyl-piperazin-1-yl)- | | 0.6 |
| | ethanone]-6-(3,4-dimethoxyphenyl)-pyrido[3,2- | | |
| | d]pyrimidine | | |
| 184/231 | 2-amino-4-(morpholino)-6-(3,4-dichlorophenyl)- | 3.9 | |
| | pyrido[3,2-d]pyrimidine | | |

Table 1 (end)

### EXAMPLES 323 TO 356

### Preparation of 2-acetamido-4-arylamino-6-(3,4-dimethoxy-phenyl)pyrido[3,2-d]pyrimidines and 2-amino-4-arylamino-6-(3,4-dimethoxyphenyl) pyrido[3,2-d]pyrimidines

The procedure of examples 26 to 36 is repeated, except for the use of other arylamines (as mentioned below for each example) as starting materials, and achieves in good yield the following 2-amino-4-arylamino-6-(3,4-dimethoxy-phenyl)pyrido[3,2-d]pyrimidines, each time through the corresponding intermediate having the 2-amino group protected in the form of acetamido:
2-amino-4-(2-bromoanilino)-6-(3,4-dimethoxy-phenyl)pyrido[3,2-d]pyrimidine (example 323) from 2-bromoaniline,
2-amino-4-(4-bromoanilino)-6-(3,4-dimethoxy-phenyl)pyrido[3,2-d]pyrimidine (example 324) from 4-bromoaniline,
2-amino-4-(2-chloroanilino)-6-(3,4-dimethoxy-phenyl)pyrido[3,2-d]pyrimidine (example 325) from 2-chloroaniline,
2-amino-4-(3-chloroanilino)-6-(3,4-dimethoxy-phenyl)pyrido[3,2-d]pyrimidine (example 326) from 3-chloroaniline,
2-amino-4-(4-chloroanilino)-6-(3,4-dimethoxy-phenyl)pyrido[3,2-d]pyrimidine (example 327) from 4-chloroaniline,
2-amino-4-(3-chloro-4-methoxyanilino)-6-(3,4-dimethoxy-phenyl)pyrido[3,2-d]pyrimidine (example 328) from 3-chloro-4-methoxyaniline,
2-amino-4-(5-chloro-2-methoxyanilino)-6-(3,4-dimethoxy-phenyl)pyrido[3,2-d]pyrimidine (example 329) from 5-chloro-2-methoxyaniline,
2-amino-4-(2,3-dimethylanilino)-6-(3,4-dimethoxy-phenyl)pyrido[3,2-d]pyrimidine (example 330) from 2,3-dimethylaniline,
2-amino-4-(2,4-dimethylanilino)-6-(3,4-dimethoxy-phenyl)pyrido[3,2-d]pyrimidine (example 331) from 2,4-dimethylaniline,
2-amino-4-(2,5-dimethylanilino)-6-(3,4-dimethoxy-phenyl)pyrido[3,2-d]pyrimidine (example 332) from 2,5-dimethylaniline,
2-amino-4-(2,6-dimethylanilino)-6-(3,4-dimethoxy-phenyl)pyrido[3,2-d]pyrimidine (example 333) from 2,6-dimethylaniline,
2-amino-4-(3,4-dimethylanilino)-6-(3,4-dimethoxy-phenyl)pyrido[3,2-d]pyrimidine (example 334) from 3,4-dimethylaniline,
2-amino-4-(2-fluoroanilino)-6-(3,4-dimethoxy-phenyl)pyrido[3,2-d]pyrimidine (example 335) from 2-fluoroaniline,
2-amino-4-(3-fluoroanilino)-6-(3,4-dimethoxy-phenyl)pyrido[3,2-d]pyrimidine (example 336) from 3-fluoroaniline,
2-amino-4-(4-fluoroanilino)-6-(3,4-dimethoxy-phenyl)pyrido[3,2-d]pyrimidine (example 337) from 4-fluoroaniline,
2-amino-4-(3-fluoro-2-methoxyanilino)-6-(3,4-dimethoxy-phenyl)pyrido[3,2-d]pyrimidine (example 338) from 3-fluoro-2-methoxyaniline,
2-amino-4-(3-fluoro-4-methoxyanilino)-6-(3,4-dimethoxy-phenyl)pyrido[3,2-d]pyrimidine (example 339) from 3-fluoro-4-methoxyaniline,
2-amino-4-(2-fluoro-4-methylanilino)-6-(3,4-dimethoxy-phenyl)pyrido[3,2-d]pyrimidine (example 340) from 2-fluoro-4-methylaniline,
2-amino-4-(2-fluoro-5-methylanilino)-6-(3,4-dimethoxy-phenyl)pyrido[3,2-d]pyrimidine (example 341) from 2-fluoro-5-methylaniline,
2-amino-4-(3-fluoro-2-methylanilino)-6-(3,4-dimethoxy-phenyl)pyrido[3,2-d]pyrimidine (example 342) from 3-fluoro-2-methylaniline,
2-amino-4-(3-fluoro-4-methylanilino)-6-(3,4-dimethoxy-phenyl)pyrido[3,2-d]pyrimidine (example 343) from 3-fluoro-4-methylaniline,
2-amino-4-(4-fluoro-2-methylanilino)-6-(3,4-dimethoxy-phenyl)pyrido[3,2-d]pyrimidine (example 344) from 4-fluoro-2-methylaniline,
2-amino-4-(5-fluoro-2-methylanilino)-6-(3,4-dimethoxy-phenyl)pyrido[3,2-d]pyrimidine (example 345) from 5-fluoro-2-methylaniline,
2-amino-4-(2-fluoro-4-iodoanilino)-6-(3,4-dimethoxy-phenyl)pyrido[3,2-d]pyrimidine (example 346) from 2-fluoro-4-iodoaniline,
2-amino-4-(2-iodoanilino)-6-(3,4-dimethoxy-phenyl)pyrido[3,2-d]pyrimidine (example 347) from 2-iodoaniline,
2-amino-4-(3-iodoanilino)-6-(3,4-dimethoxy-phenyl)pyrido[3,2-d]pyrimidine (example 348) from 3-iodoaniline,
2-amino-4-(4-iodoanilino)-6-(3,4-dimethoxy-phenyl)pyrido[3,2-d]pyrimidine (example 349) from 4-iodoaniline,
2-amino-4-(2-methoxy-5-methylanilino)-6-(3,4-dimethoxy-phenyl)pyrido[3,2]pyrimidine (example 350) from 2-methoxy-5-methylaniline,
2-amino-4-(4-methoxy-2-methylanilino)-6-(3,4-dimethoxy-phenyl)pyrido[3,2-d]pyrimidine (example 351) from 4-methoxy-2-methylaniline,
2-amino-4-(5-methoxy-2-methylanilino)-6-(3,4-dimethoxy-phenyl)pyrido[3,2-d]pyrimidine (example 352) from 5-methoxy-2-methylaniline,
2-amino-4-(2-ethoxyanilino)-6-(3,4-dimethoxy-phenyl)pyrido[3,2-d]pyrimidine (example 353) from 2-ethoxyaniline (o-phenetidine),
2-amino-4-(3-ethoxyanilino)-6-(3,4-dimethoxy-phenyl)pyrido[3,2-d]pyrimidine (example 354) from 3-ethoxyaniline (m-phenetidine),
2-amino-4-(4-ethoxyanilino)-6-(3,4-dimethoxy-phenyl)pyrido[3,2-d]pyrimidine (example 355) from 4-ethoxyaniline (p-phenetidine), and
2-amino-4-(α-naphthylamino)-6-(3,4-dimethoxy-phenyl)pyrido[3,2-d]pyrimidine (example 356) from α-naphthylamine.

### EXAMPLES 357 TO 367

### Preparation of 2-acetamido-4-arylalkylamino-6-(3,4-dimethoxyphenyl)pyrido[3,2-d]pyrimidines and 2-amino-4-arylalkylamino-6-(3,4-dimethoxyphenyl) Pyrido[3,2-d]pyrimidines

The procedure of examples 26 to 36 is repeated, except for the use of other arylalkylamines (as mentioned below for each example) as starting materials, and achieves in good yield the following 2-amino-4-arylalkylamino-6-(3,4-dimethoxy-phenyl)pyrido[3,2-d]pyrimidines, each time through the corresponding intermediate having the 2-amino group protected in the form of acetamido:
2-amino-4-benzylamino-6-(3,4-dimethoxyphenyl)pyrido[3,2-d]pyrimidine (example 357) from benzylamine,
2-amino-4-(2-methoxybenzylamino)-6-(3,4-dimethoxyphenyl)pyrido[3,2-d]pyrimidine (example 358) from 2-methoxybenzylamine,
2-amino-4-(3-methoxybenzylamino)-6-(3,4-dimethoxyphenyl)pyrido[3,2-d]pyrimidine (example 359) from 3-methoxybenzylamine,
2-amino-4-(4-methoxybenzylamino)-6-(3,4-dimethoxyphenyl)pyrido[3,2-d]pyrimidine (example 210) from 4-methoxybenzylamine,
2-amino-4-(2-fluorobenzylamino)-6-(3,4-dimethoxyphenyl)pyrido[3,2-d]pyrimidine (example 360) from 2-fluorobenzylamine,
2-amino-4-(3-fluorobenzylamino)-6-(3,4-dimethoxyphenyl)pyrido[3,2-d]pyrimidine (example 361) from 3-fluorobenzylamine,
2-amino-4-(4-fluorobenzylamino)-6-(3,4-dimethoxyphenyl)pyrido[3,2-d]pyrimidine (example 362) from 4-fluorobenzylamine,
2-amino-4-(2-chlorobenzylamino)-6-(3,4-dimethoxyphenyl)pyrido[3,2-d]pyrimidine (example 363) from 2-chlorobenzylamine,
2-amino-4-(3-chlorobenzylamino)-6-(3,4-dimethoxyphenyl)pyrido[3,2-d]pyrimidine (example 364) from 3-chlorobenzylamine,
2-amino-4-(4-chlorobenzylamino)-6-(3,4-dimethoxyphenyl)pyrido[3,2-d]pyrimidine (example 365) from 4-chlorobenzylamine,
2-amino-4-(2-aminobenzylamino)-6-(3,4-dimethoxyphenyl)pyrido[3,2-d]pyrimidine (example 366) from 2-aminobenzylamine,
2-amino-4-diphenylmethylamino-6-(3,4-dimethoxyphenyl)pyrido[3,2-d]pyrimidine (example 367) from aminodiphenylmethane,

### EXAMPLES 368 TO 378

### Preparation of 2-acetamido-4-alkylamino-6-(3,4-dimethoxy-phenyl)pyrido[3,2-d]pyrimidines and 2-amino-4-alkylamino-6-(3,4-dimethoxyphenyl) pyrido[3,2-d]pyrimidines

The procedure of examples 26 to 36 is repeated, except for the use of other alkylamines (as mentioned below for each example) as starting materials, and achieves in good yield the following 2-amino-4-alkylamino-6-(3,4-dimethoxy-phenyl)pyrido[3,2-d]pyrimidines, each time through the corresponding intermediate having the 2-amino group protected in the form of acetamido:
2-amino-4-(1,2-diaminopropyl)-6-(3,4-dimethoxyphenyl)pyrido[3,2-d]pyrimidine (example 368) from 1,2-diaminopropane,
2-amino-4-(1,3-diaminopropyl)-6-(3,4-dimethoxyphenyl)pyrido[3,2-d]pyrimidine (example 369) from 1,3-diaminopropane,
2-amino-4-(1,4-diaminobutyl)-6-(3,4-dimethoxyphenyl)pyrido[3,2-d]pyrimidine (example 370) from 1,4-diaminobutane,
2-amino-4-(1,5-diaminopentyl)-6-(3,4-dimethoxyphenyl)pyrido[3,2-d]pyrimidine (example 371) from 1,5-diaminopentane,
2-amino-4-(1,6-diaminohexyl)-6-(3,4-dimethoxyphenyl)pyrido[3,2-d]pyrimidine (example 372) from 1,6-diaminohexane,
2-amino-4-(1,2-diaminocyclohexyl)-6-(3,4-dimethoxyphenyl)pyrido[3,2-d]pyrimidine (example 373) from 1,2-diaminocyclohexane,
2-amino-4-(1,7-diaminoheptyl)-6-(3,4-dimethoxyphenyl)pyrido[3,2-d]pyrimidine (example 374) from 1,7-diaminoheptane,
2-amino-4-(1,8-diaminooctyl)-6-(3,4-dimethoxyphenyl)pyrido[3,2-d]pyrimidine (example 375) from 1,8-diaminooctane,
2-amino-4-(1,9-diaminononyl)-6-(3,4-dimethoxyphenyl)pyrido[3,2-d]pyrimidine (example 376) from 1,9-diaminononane,
2-amino-4-(1,10-diaminodecyl)-6-(3,4-dimethoxyphenyl)pyrido[3,2-d]pyrimidine (example 377) from 1,10-diaminodecane, and
2-amino-4-(1,12-diaminododecyl)-6-(3,4-dimethoxyphenyl)pyrido[3,2-d]pyrimidine (example 378) from 1,12-diaminododecane.

### EXAMPLE 379

### Phosphodiesterase-4 Inhibiting Activity

A phosphodiesterase-4 (PDE-4) extract was prepared from cultured U937 cells, then cells were lysed and homogenised. Following homogenization, the supernatant was collected by centrifugation and loaded onto a Sephacryl S-200 column. Fractions found to contain PDE-4 activity were used in the subsequent assay procedure.

PDE-4 inhibitory activity of some of the pyrido[3,2-d]pyrimidine derivatives described in the previous examples has been assessed using an isotopic two-step method as follows. The derivative to be tested (in 1% DMSO) was combined with 0.2 μg of PDE-4 enzyme and preincubated for 15 minutes at 25° C. in a buffer containing 50 mM Tris-HCl and 5 mM MgCl₂ at pH 7.5. Radiolabelled cyclic [³H]AMP+cAMP was then added to provide a final concentration of 1.01 μM and incubated for 20 minutes at 25° C. Active PDE-4 enzyme hydrolyses the cyclic [³H]AMP into 5′-[³H]AMP. The reaction was terminated by incubating the reaction mixture at 100° C. Snake venom from Crotalus atrox (10 μl of 10 mg/ml) was added for 10 minutes at 37° C. for further hydrolyzing 5′-[³H]AMP into [³H]adenosine by the effect of nucleotidase contained in said snake venom. The reaction was then terminated by the addition of 200 μL of an anion exchange resin (AG1-X2) which binds all charged nucleotides except [³H]adenosine. The resin was allowed to settle for 5 minutes and then 50 μl of the aqueous phase was taken and combined with 0.2 ml of scintillation fluid. The radioactivity of the solution was measured using a liquid scintillation counter.

Table 2 shows IC₅₀ values (expressed in μM), or the percentage inhibition at a certain concentration, of some derivatives of the previous examples which have been tested in this assay.

TABLE 2

| Example | Derivative | PDE-4 (% inhibition) |
|---|---|---|
| 8 | 4-(4-[3-methylphenyl)amino]carbonyl]piperazin-1-yl)- | 0.016 (IC₅₀) |
| | 6-(3,4-dimethoxyphenyl)-pyrido[3,2-d]pyrimidine | |
| 11 | 2-methyl-4-(4-[3- | 69% @ 0.5 μM |
| | methylphenyl)amino]carbonyl]pipera-zin-1-yl)-6-(3,4- | |
| | dimethoxyphenyl)-pyrido[3,2-d]pyrimi-dine | |
| 24 | 2-amino-4-isopropoxy-6-(3,4-dimethoxyphenyl)- | 29% @ 0.5 μM |
| | pyrido[3,2-d]pyrimidine | |
| 26 | 2-amino-4-[(4-carboxylic ethyl ester)-piperidin-1-yl]- | 0.25 (IC₅₀) |
| | 6-(3,4-dimethoxyphenyl)-pyrido[3,2-d]pyrimidine | |
| 41 | 2-amino-4-morpholino-6-(3,4-dimethoxyphenyl)- | 0.041 (IC₅₀) |
| | pyrido[3,2-d]pyrimidine | |
| 42 | 2-amino-4-(4-[3- | 0.061 (IC₅₀) |
| | methylphenyl)amino]carbonyl]pipera-zin-1-yl)-6-(3,4- | |
| | dimethoxyphenyl)-pyrido[3,2-d]pyrimi-dine | |
| 43 | 2-amino-4-(4-fluorophenyl-piperazin-1-yl)-6-(3,4- | 61% @ 0.09 μM |
| | dimethoxyphenyl)-pyrido[3,2-d]pyrimidine | |
| 45 | 2-amino-4-(phenoxyethyl-piperazin-1-yl)-6-(3,4- | 76% @ 0.7 μM |
| | dimethoxyphenyl)-pyrido[3,2-d]pyrimidine | |
| 47 | 2-amino-4-(2-pyridyl-piperazin-1-yl)-6-(3,4- | 64% @ 0.05 μM |
| | dimethoxyphenyl)-pyrido[3,2-d]pyrimidine | |
| 48 | 2-amino-4-[2-(piperazin-1-yl)-acetic acid N-(2- | 59% @ 0.06 μM |
| | thiazolyl)-amide]-6-(3,4-dimethoxyphenyl)-pyrido[3,2- | |
| | d]pyrimidine | |
| 49 | 2-amino-4-(N-acetyl-piperazin-1-yl)-6-(3,4- | 48% @ 0.04 μM |
| | dimethoxy-phenyl)-pyrido[3,2-d]pyrimidine | |
| 51 | 2-amino-4-[1-(2-furoyl)-piperazin-1-yl]-6-(3,4- | 63% @ 0.03 μM |
| | dimethoxy-phenyl)-pyrido[3,2-d]pyrimidine | |
| 54 | 2-amino-4(N-3-thienyl-carbamoyl-piperazin-1-yl)-6- | 62% @ 0.1 μM |
| | (3,4-dimethoxyphenyl)-pyrido[3,2-d]pyrimidine | |
| 56 | 2-amino-4(N-4-fluorophenyl-carbamoyl-piperazin-1- | 74% @ 0.1 μM |
| | yl)-6-(3,4-dimethoxy-phenyl)-pyrido[3,2-d]pyrimidine | |
| 57 | 2-amino-4(N-3-chloro-4-fluorophenyl-carbamoyl- | 74% @ 0.1 μM |
| | piperazin-1-yl)-6-(3,4-dimethoxyphenyl)-pyrido[3,2- | |
| | d]pyrimidine | |
| 58 | 2-amino-4(N-3-chloro-phenyl-carbamoyl-piperazin-1- | 75% @ 0.1 μM |
| | yl)-6-(3,4-dimethoxyphenyl)-pyrido[3,2-d]pyrimidine | |
| 59 | 2-amino-4[(N-4-chloro-phenoxy-acetyl)-piperazin-1- | 0.034 (IC₅₀) |
| | yl]-6-(3,4-dimethoxyphenyl)-pyrido[3,2-d]pyrimidine | |
| 79 | 4-[(N-3-chlorophenylcarbamoyl)-piperazin-1-yl]-6-(3- | 67% @ 10 μM |
| | chloro-4-methoxyphenyl)-pyrido[3,2-d]pyrimidine | |
| 80 | 4-[(N-3-chlorophenylcarbamoyl)-piperazin-1-yl]-6- | 17% @ 10 μM |
| | (1,4-benzodioxan-6-yl)-pyrido[3,2-d]pyrimidine | |
| 81 | 4-[(N-3-chlorophenylcarbamoyl)-piperazin-1-yl]-6- | 12% @ 10 μM |
| | (3,4-dimethylphenyl)-pyrido[3,2-d]pyrimidine | |
| 82 | 4-[(N-3-chlorophenylcarbamoyl)-piperazin-1-yl]-6- | 31% @ 10 μM |
| | (3,4-methylenedioxy)phenyl-pyrido[3,2-d]pyrimidine | |
| 83 | 4-[(N-3-chlorophenylcarbamoyl)-piperazin-1-yl]-6-(3- | 49% @ 10 μM |
| | chloro-4-ethoxyphenyl)-pyrido[3,2-d]pyrimidine | |
| 84 | 4-[(N-3-chlorophenylcarbamoyl)-piperazin-1-yl]-6- | 22% @ 10 μM |
| | (3,4-dichlorophenyl)-pyrido[3,2-d]pyrimidine | |
| 91 | 2-(p-tolylamino-4-[(N-3-methyl-phenylcarbamoyl)- | 10 μM (IC₅₀) |
| | piperazin-1-yl]-6-(3,4-dimethoxyphenyl)-pyrido[3,2-d] | |
| | pyrimidine | |
| 108 | 4-(N-3-chloro-4-fluoro-phenylcarbamoyl-piperazin-1- | 59% @ 0.2 μM |
| | yl)-6-(3,4-dimethoxyphenyl)-pyrido[3,2-d]pyrimidine | |
| 109 | 4-[N-2-thienyl-carbamoyl)-piperazin-1-yl]-6-(3,4- | 58% @ 0.09 μM |
| | dimethoxyphenyl)-pyrido[3,2-d]pyrimidine | |
| 112 | 4-[N-3-chlorophenyl-carbamoyl)-piperazin-1-yl]-6- | 56% @ 0.07 μM |
| | (3,4-dimethoxyphenyl)-pyrido[3,2-d]pyrimidine | |
| 113 | 4-[(N-4-chlorophenoxy-acetyl)-piperazin-1-yl]-6-(3,4- | 77% @ 0.4 μM |
| | dimethoxyphenyl)-pyrido[3,2-d]pyrimidine | |
| 117 | 4-[(N-3-chloro-phenylcarbamoyl)-piperazin-1-yl]-6-(3- | 54% @ 10 μM |
| | methyl-4-methoxyphenyl)-pyrido[3,2-d]pyrimidine | |
| 118 | 4-[(N-4-chloro-phenylcarbamoyl)-piperazin-1-yl]-6-(3- | 62% @ 10 μM |
| | methyl-4-methoxyphenyl)-pyrido[3,2-d]pyrimidine | |
| 120 | 4-[(N-3-chloro-phenylcarbamoyl)-piperazin-1-yl]-6-(3- | 67% @ 0.8 μM |
| | methoxy-4-ethoxyphenyl)-pyrido[3,2-d]pyrimidine | |
| 121 | 4-[(N-3-chloro-phenylcarbamoyl)-piperazin-1-yl]-6-(3- | 54% 10 μM |
| | methoxy-4-isopropoxy-phenyl-pyrido[3,2- | |
| | d]pyrimidine | |
| 157 | 2-amino-4[(N-3-chloro-phenyl-carbamoyl)-piperazin- | 57% @ 0.9 μM |
| | 1-yl]-6-(3-chloro-4-methoxy-phenyl)pyrido-[3,2- | |
| | d]pyrimi-dine | |
| 159 | 2-amino-4[(N-3-chloro-phenyl-carbamoyl)-piperazin- | 16% @ 0.1 μM; 71% |
| | 1-yl]-6-(3-fluoro-4-ethoxy-phenyl)pyrido-[3,2- | @ 10 μM; IC₅₀ = |
| | | 2.44 μM |
| | d]pyrimidine | |
| 161 | 2-amino-4[(N-3-chloro-phenyl-carbamoyl)-piperazin- | 68% @ 10 μM |
| | 1-yl]-6-(3,4-methylenedioxy)phenyl)pyrido-[3,2- | |
| | d]pyrimi-dine | |
| 162 | 2-amino-4[(N-3-chloro-phenyl-carbamoyl)-piperazin- | 63% @ 10 μM; IC₅₀ = |
| | 1-yl]-6-(1,4-benzodioxane-phenyl)pyrido-[3,2- | 7.84 μM |
| | d]pyrimi-dine | |
| 189 | 2-amino-4-[(N-4-chloro-benzylcarbamoyl)-piperazin- | 32% @ 10 μM |
| | 1-yl]-6-(4-fluorophenyl)-pyrido[3,2-d]pyrimidine | |
| 207 | 2-amino-4-[(N-4-chloro-phenoxy-acetyl)-piperazin-1- | 34% @ 10 μM |
| | yl]-6-(4-fluorophenyl)-pyrido[3,2-d]pyrimidine | |
| 211 | 4-[N-(3-chloro-phenylcarbamoyl)-piperazin-1-yl]-6-(3- | 16% @ 10 μM |
| | methoxy-4-cyclopropylmethoxy-phenyl)-pyrido[3,2- | |
| | d]pyrimidine | |
| 212 | 4-[N-(3-chloro-phenylcarbamoyl)-piperazin-1-yl]-6-(3- | 43% @ 0.9 μM |
| | hydroxy-4-methoxy-phenyl)-pyrido[3,2-d]pyrimidine | |
| 213 | 4-[N-(3-chloro-phenylcarbamoyl)-piperazin-1-yl]-6-(3- | 78% @ 0.06 μM |
| | ethoxy-4-methoxy-phenyl)-pyrido[3,2-d]pyrimidine | |
| 214 | 4-[N-(3-chloro-phenylcarbamoyl)-piperazin-1-yl]-6-(3- | 61% @ 0.3 μM |
| | isopropoxy-4-methoxy-phenyl)-pyrido[3,2- | |
| | d]pyrimidine | |
| 261 | 2-amino-4-[(3-methylphenylcarbamoyl)- | 86% @ 10 μM |
| | ethylenediamine-1-N-yl]-6-(3,4-dimethoxyphenyl)- | |
| | pyrido[3,2-d]pyrimidine | |

Table 2 (end)

### EXAMPLE 380

### Anti-HCV Assay/Replicon Assay

Huh-5-2 cells [a cell line with a persistent HCV replicon 13891 uc-ubi-neo/NS3-3′/5.1; replicon with firefly luciferase-ubiquitin-neomycin phosphotransferase fusion protein and EMCV-IRES driven NS3-5B HCV polyprotein] was cultured in a RPMI medium (commercially available from Gibco) supplemented with 10% fetal calf serum, 2 mM L-glutamine (commercially available from Life Technologies), 1×non-essential amino acids (commercially available from Life Technologies); 100 IU/ml penicillin, 100 μg/ml streptomycin and 250 μg/ml G418 (Geneticin, commercially available from Life Technologies). Cells were seeded at a density of 7,000 cells per well in 96 well View Plate (commercially available from Packard) in a medium containing the same components as described above, except for G418. Cells were allowed to adhere and proliferate for 24 hours. At that time, the culture medium was removed and serial dilutions of the pyrido[3,2-d]pyrimidine derivatives to be tested were added in a culture medium lacking G418. Interferon-α 2a (500 IU) was included as a positive control. Plates were further incubated at 37° C. and 5% CO₂ for 72 hours. Replication of the HCV replicon in Huh-5 cells resulted in luciferase activity in the cells. Luciferase activity was measured by adding 50 μl of 1×Glo-lysis buffer (commercially available from Promega) for 15 minutes followed by 50 μl of the Steady-Glo Luciferase assay reagent (commercially available from Promega). Luciferase activity was measured with a luminometer and the signal in each individual well was expressed as a percentage of the untreated cultures. Parallel cultures of Huh-5-2 cells, seeded at a density of 7,000 cells/well of classical 96-well cel culture plates (commercially available from Becton-Dickinson) were treated in a similar fashion except that no Glo-lysis buffer or Steady-Glo Luciferase reagent was added. Instead the density of the culture was measured by means of the MTS method (commercially available from Promega).

Results in table 3 are expressed by the following data:
the 50% cytostatic concentration (CC₅₀), i.e. the concentration that results in 50% inhibition of cell growth, and
the 50% effective concentration (EC₅₀), i.e. the concentration that protects 50% of the cell monolayer from virus-induced cythopathic effect.
   Table 3 shows EC₅₀ and CC₅₀ values (expressed in μM, i.e. μmol/l) of a few derivatives tested in this assay.

TABLE 3

| Derivative | EC₅₀ | CC₅₀ |
|---|---|---|
| Example 27 | 0.5 | >50 |
| Example 36 | 1.0 | 7.7 |

### EXAMPLE 381

### Synthesis of 2-amino-4-oxo-6-(4-fluorophenyl)-pyrido-[3,2-d]-pyrimidine

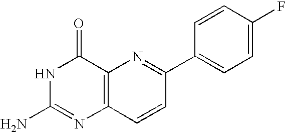

To a suspension of 2-amino-6-chloro-3H-pyrido[3,2-d]pyrimidin-4-one (2.55 g, 13.0 mmol) in dioxane (190 ml) and water (40 ml), potassium carbonate (7.22 g, 52.2 mmol) and 4-fluorophenylboronic acid (2.02 g, 14.4 mmol) were added and the mixture was purged with nitrogen for 15 minutes. Upon addition of tetrakis (triphenylphosphine)palladium(0) (750 mg, 0.65 mmol), the reaction mixture was heated at reflux temperature under a nitrogen atmosphere for 18 hours. The cooled mixture was filtered through Celite 545 and the filtrate was acidified with 6 M hydrochloric acid till pH 5-6 (as measured with indicator paper). The resulting suspension was kept at 4° C. overnight and the yellow precipitate (389 mg) was filtered off, washed twice with cold water and dried. The filtrate was concentrated under reduced pressure and worked up in the same way to yield another crop of the pure (by TLC on silica plates developed in 15% methanol in dichloromethane) title product (1.58 g) along with an impure fraction. The latter was purified by column chromatography on silica (10% methanol 1% triethylamine in dichloromethane) to afford a further 168 mg of the title compound. The overall yield was 2.14 g (64%) for the title compound which was characterised as follows: MS (m/z): 257 ([M+H]⁺, 100).

### EXAMPLE 382

### Synthesis of 2-amino-4-(N-piperazin-1-yl)-6-(4-fluorophenyl)-pyrido-[3,2-d]pyrimidine

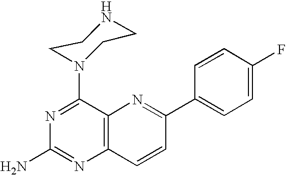

A suspension of 2-amino-4-oxo-6-(4-fluorophenyl)-pyrido-[3,2-d]pyrimidine (1.97 g, 7.69 mmol), piperazine (2.69 g, 31.2 mmol), p-toluenesulfonic acid monohydrate (195 mg, 1.0 mmol), ammonium sulfate (156 mg, 1.2 mmol) and 1,1,1,3,3,3-hexamethyldisilazane (HMDS; 8.5 ml, 40.3 mmol) in pyridine (40 ml) was heated at reflux for 4 days. Another aliquot of piperazine was added and the reaction mixture was heated at reflux for one more day. Upon cooling, the reaction mixture was evaporated with silica gel and purified twice on a silica gel column (15-20% methanol and 1% triethylamine in dichloromethane) to afford the title compound (1.82 g, 73%) which was characterised as follows: MS (m/z): 325 ([M+H]⁺, 100).

### EXAMPLE 383

### Synthesis of 2-amino-4-[4-(2-naphthoxyacetyl)piperazin-1-yl]-6-(4-fluorophenyl)-pyrido[3,2-d]pyrimidine

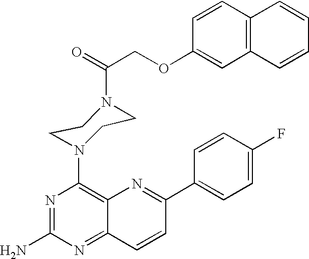

A solution of 2-amino-4-(N-piperazin-1-yl)-6-(4-fluorophenyl)-pyrido[3,2-d]pyrimidine (40 mg, 0.12 mmol), 2-naphthoxyacetic acid (34 mg, 0.16 mmol), diisopropylethylamine (hereinafter referred as DIPEA; 0.33 mmol) and O-(benzotriazol-1-yl)-N,N,N′,N′-tetramethyluronium tetrafluoroborate (TBTU; 66 mg, 0.21 mmol) in dry DMF (2 ml) was stirred under an N₂ atmosphere for 3 hours at room temperature. The reaction mixture was applied directly onto a plate of silica gel. Developing with 8% methanol in dichloromethane afforded the title compound (21 mg, 28%) which was characterised as follows: MS (m/z): 509 ([M+H]⁺, 100).

### EXAMPLE 384

### Synthesis of 2-amino-4-[4-(3-methylphenoxyacetyl)piperazin-1-yl]-6-(4-fluorophenyl)-pyrido[3,2-o]pyrimidine

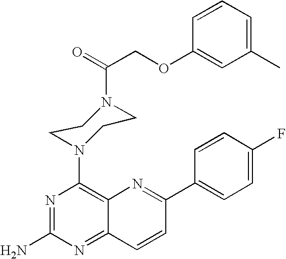

This compound was prepared according to the procedure of example 383, using (3-methylphenoxy)acetic acid and 2-amino-4-(N-piperazin-1-yl)-6-(4-fluorophenyl)-pyrido[3,2-d]pyrimidine as starting materials. Yield: 54% for the title compound which was characterised as follows: MS (m/z): 473 ([M+H]⁺, 100).

### EXAMPLE 385

### Synthesis of 2-amino-4-[4-(3-chlorophenoxyacetyl)piperazin-1-yl]-6-(4-fluorophenyl)-pyrido[3,2-d]pyrimidine

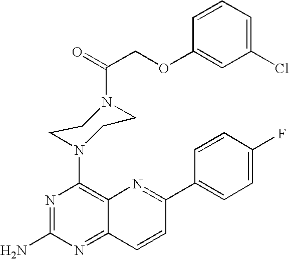

This compound was prepared according to the procedure of example 383, using 3-chlorophenoxyacetic acid and 2-amino-4-(N-piperazin-1-yl)-6-(4-fluorophenyl)-pyrido[3,2-d]pyrimidine as starting materials. Yield: 34% for the title compound which was characterised as follows: MS (m/z): 493, 495 ([M+H]⁺, 100).

### EXAMPLE 386

### Synthesis of 2-amino-4-[4-(2,4-dichlorophenoxyacetyl)piperazin-1-yl]-6-(4-fluorophenyl)-pyrido[3,2-d]pyrimidine

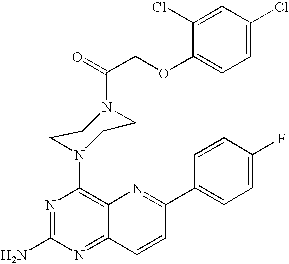

This compound was prepared according to the procedure of example 383, using 2,4-dichlorophenoxyacetic acid and 2-amino-4-(N-piperazin-1-yl)-6-(4-fluorophenyl)-pyrido[3,2-d]pyrimidine as starting materials. Yield: 41% for the title compound which was characterised as follows: MS (m/z): 527, 529 ([M+H]⁺, 100).

### EXAMPLE 387

### Synthesis of 2-amino-4-[4-(4-fluorophenoxyacetyl)piperazin-1-yl]-6-(4-fluorophenyl)-pyrido[3,2-d]pyrimidine

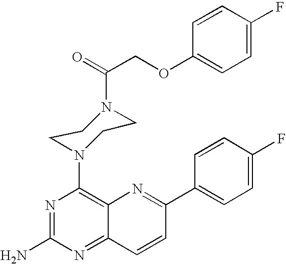

This compound was prepared according to the procedure of example 383, using 4-fluorophenoxyacetic acid and 2-amino-4-(N-piperazin-1-yl)-6-(4-fluorophenyl)-pyrido[3,2-d]pyrimidine as starting materials. Yield: 9% for the title compound which was characterised as follows: MS (m/z): 477 ([M+H]⁺, 100).

### EXAMPLE 388

### Synthesis of 2-amino-4-[4-(4-bromophenoxyacetyl)piperazin-1-yl]-6-(4-fluorophenyl)-pyrido[3,2-d]pyrimidine

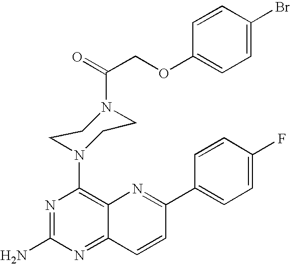

This compound was prepared according to the procedure of example 383, using 4-bromophenoxyacetic acid and 2-amino-4-(N-piperazin-1-yl)-6-(4-fluorophenyl)-pyrido[3,2-d]pyrimidine as starting materials. Yield: 17% for the title compound which was characterised as follows: MS (m/z): 537, 539 ([M+H]⁺, 100).

### EXAMPLE 389

### Synthesis of 2-amino-4-[4-(trimethylacetyl)piperazin-1-yl]-6-(3,4-dimethoxyphenyl)-pyrido[3,2-d]pyrimidine

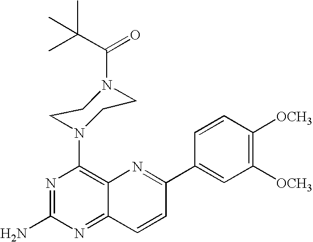

Triethylamine (15 μl) was added to a solution of 2-amino-4-(N-piperazin-1-yl)-6-(3,4-dimethoxyphenyl)-pyrido[3,2-d]pyrimidine (35 mg) in dichloromethane (2 ml), followed by trimethylacetyl chloride (13 μl). After stirring for 30 minutes at room temperature, the reaction mixture was applied directly onto a plate of silica gel. Elution with mixtures of dichloromethane and methanol (6-10% MeOH in CH₂Cl₂) yielded the title compound (41 mg, 95%) which was characterised as follows: MS (m/z): 451 ([M+H]⁺, 100).

### EXAMPLE 390

### Synthesis of 2-amino-4-[4-(4-pentenoyl)piperazin-1-yl]-6-(3,4-dimethoxyphenyl)-pyrido[3,2-d]pyrimidine

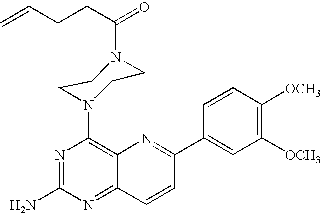

This compound was prepared according to the procedure of example 389, using 4-pentenoyl chloride and 2-amino-4-(N-piperazin-1-yl)-6-(3,4-dimethoxyphenyl)-pyrido[3,2-d]pyrimidine as starting materials. Yield: 100% for the title compound which was characterised as follows: MS (m/z): 449 ([M+H]⁺, 100).

### EXAMPLE 391

### Synthesis of 2-amino-4-[4-(2-methylpropionyl)piperazin-1-yl]-6-(3,4-dimethoxyphenyl)-pyrido[3,2-d]pyrimidine

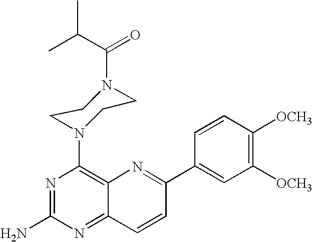

This compound was prepared according to the procedure of example 389, using isobutyryl chloride and 2-amino-4-(N-piperazin-1-yl)-6-(3,4-dimethoxyphenyl)-pyrido[3,2-d]pyrimidine as starting materials. Yield: 79% for the title compound which was characterised as follows: MS (m/z): 437 ([M+H]⁺, 100).

### EXAMPLE 392

### Synthesis of 2-amino-4-[4-(3,3-dimethylbutyryl)piperazin-1-yl]-6-(3,4-dimethoxyphenyl)-pyrido[3,2-d]pyrimidine

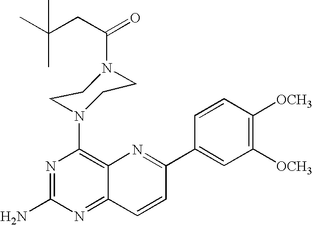

This compound was prepared according to the procedure of example 389, using tert-butylacetyl chloride and 2-amino-4-(N-piperazin-1-yl)-6-(3,4-dimethoxyphenyl)-pyrido[3,2-d]pyrimidine as starting materials. Yield: 61% for the title compound which was characterised as follows: MS (m/z): 465 ([M+H]⁺, 100).

### EXAMPLE 393

### Synthesis of 2-amino-4-[4-(2-propenoyl)piperazin-1-yl]-6-(3,4-dimethoxyphenyl)-pyrido[3,2-d]pyrimidine

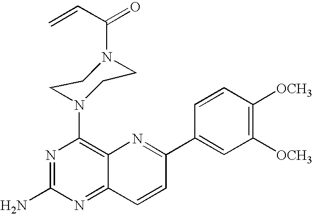

This compound was prepared according to the procedure of example 389, using acryloyl chloride and 2-amino-4-(N-piperazin-1-yl)-6-(3,4-dimethoxyphenyl)-pyrido[3,2-d]pyrimidine as starting materials. Yield: 100% for the title compound which was characterised as follows: MS (m/z): 421 ([M+H]⁺, 100).

### EXAMPLE 394

### Synthesis of 2-amino-4-[4-(N,N-dimethylthiocarbamoyl)-piperazin-1-yl]-6-(3,4-dimethoxy-phenyl)-pyrido[3,2-d]pyrimidine

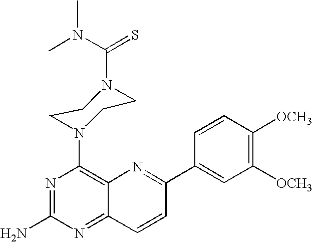

This compound was prepared according to the procedure of example 389, using dimethylthiocarbamoyl chloride and 2-amino-4-(N-piperazin-1-yl)-6-(3,4-dimethoxyphenyl)-pyrido[3,2-d]pyrimidine as starting materials. Yield: 72% for the title compound which was characterised as follows: MS (m/z): 454 ([M+H]⁺, 100).

### EXAMPLE 395

### Synthesis of 2-amino-4-[4-(N,N-dimethylcarbamoyl)-piperazin-1-yl]-6-(3,4-dimethoxy-phenyl)-pyrido[3,2-d]pyrimidine

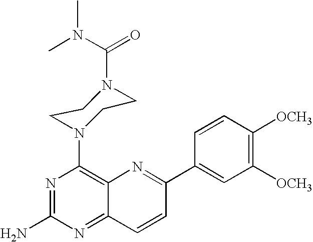

This compound was prepared according to the procedure of example 389, using dimethylcarbamoyl chloride and 2-amino-4-(N-piperazin-1-yl)-6-(3,4-dimethoxyphenyl)-pyrido[3,2-d]pyrimidine as starting materials. Yield: 98% for the title compound which was characterised as follows: MS (m/z): 438 ([M+H]⁺, 100).

### EXAMPLE 396

### Synthesis of 2-amino-4-[4-[N-(tert-butoxycarbonyl)-glycyl]-piperazin-1-yl]-6-(3,4-di-methoxyphenyl)-pyrido[3,2-d]pyrimidine

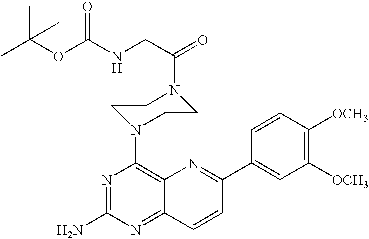

A solution of 2-amino-4-(N-piperazin-1-yl)-6-(3,4-dimethoxyphenyl)-pyrido[3,2-d]pyrimidine (100 mg, 0.27 mmol), N-(tert-butoxycarbonyl)glycine (54 mg, 0.31 mmol), DIPEA (115 μl, 0.70 mmol) and TBTU (140 mg, 0.44 mmol) in dry DMF (3 ml) was stirred under an N₂ atmosphere for 4 hours at room temperature. The reaction mixture was applied directly onto a plate of silica gel. Elution with methanol in dichloromethane (6-8% MeOH in CH₂Cl₂) yielded the title compound (27 mg, 19%) which was characterised as follows: MS (m/z): 524 ([M+H]⁺, 100).

### EXAMPLE 397

### Synthesis of 2-amino-4-[4-(N-butylcarbamoyl)-piperazin-1-yl]-6-(3,4-dimethoxyphenyl)-pyrido[3,2-d]pyrimidine

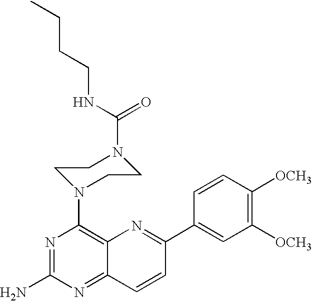

n-butyl isocyanate (12 μl, 110 μmol) was added to a solution of 2-amino-4-(N-piperazin-1-yl)-6-(3,4-dimethoxyphenyl)-pyrido[3,2-d]pyrimidine (35 mg, 96 μmol) in dichloromethane (2 ml). After stirring for approximately 45 min at room temperature, the reaction mixture was applied directly onto a plate of silica gel. Elution with 10% MeOH in CH₂Cl₂ yielded the title compound (38 mg, 100%) which was characterised as follows: MS (m/z): 466 ([M+H]⁺, 100).

### EXAMPLE 398

### Synthesis of 2-amino-4-[4-(N-hexylcarbamoyl)-piperazin-1-yl]-6-(3,4-dimethoxyphenyl)-pyrido[3,2-d]pyrimidine

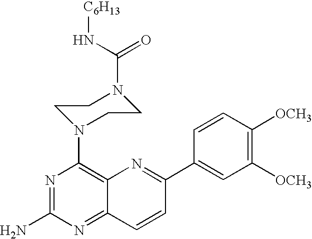

This compound was prepared according to the procedure of example 397, using n-hexyl isocyanate and 2-amino-4-(N-piperazin-1-yl)-6-(3,4-dimethoxyphenyl)-pyrido[3,2-d]pyrimidine as starting materials. Yield: 97% for the title compound which was characterised as follows: MS (m/z): 494 ([M+H]⁺, 100).

### EXAMPLE 399

### Synthesis of 2-amino-4-[4-(4-chlorophenoxyacetyl)piperazin-1-yl]-6-(4-trifluoromethylphenyl)-pyrido-[3,2-d]pyrimidine

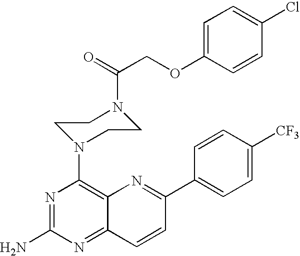

A suspension of 2-amino-4-[4-(4-chlorophenoxyacetyl)piperazin-1-yl]-6-chloro-pyrido-[3,2-d]pyrimidine (40 mg, 92 μmol), potassium fluoride (22 mg, 0.37 mmol) and 4-(trifluoromethyl)phenylboronic acid (21 mg, 0.11 mmol) in dioxane (2 ml) and water (0.5 ml) was purged with nitrogen for 15 minutes. Tetrakis (triphenylphosphine)palladium(0) (8 mg, 7 μmol) was added and the reaction mixture was heated at reflux for 1 hour under an N₂ atmosphere. Upon cooling, the mixture was applied directly onto a plate of silica gel. Elution with 5% methanol in dichloromethane afforded the title compound (49 mg, 98%) which was characterised as follows: MS (m/z): 543, 545 ([M+H]⁺, 100).

### EXAMPLE 400

### Synthesis of 2-amino-4-[4-(4-chlorophenoxyacetyl)piperazin-1-yl]-6-(4-cyanophenyl)pyrido-[3,2-d]pyrimidine

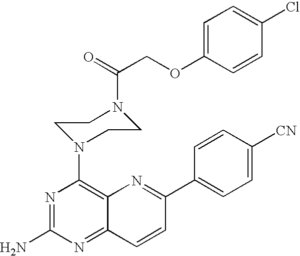

This compound was prepared according to the procedure of example 399, using 4-cyanophenylboronic acid, 2-amino-4-[4-(4-chlorophenoxyacetyl)piperazin-1-yl]-6-chloro-pyrido-[3,2-d]pyrimidine and potassium carbonate (52 mg, 0.37 mmol) as starting materials. Yield: 18% for the title compound which was characterised as follows: MS (m/z): 500, 502 ([M+H]⁺, 100).

### EXAMPLE 401

### Synthesis of 2-amino-4-[4-(4-chlorophenoxyacetyl)piperazin-1-yl]-6-(3-fluorophenyl)pyrido-[3,2-d]pyrimidine

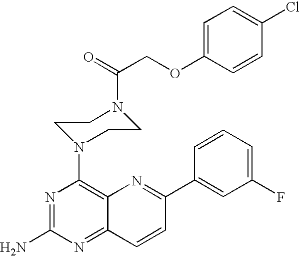

This compound was prepared according to the procedure of example 399, using 3-fluorophenylboronic acid and 2-amino-4-[4-(4-chlorophenoxyacetyl)piperazin-1-yl]-6-chloro-pyrido-[3,2-d]pyrimidine as starting materials. Yield: 80% for the title compound which was characterised as follows: MS (m/z): 493, 495 ([M+H]⁺, 100).

### EXAMPLE 402

### Synthesis of 2-amino-4-[4-(4-chlorophenoxyacetyl)piperazin-1-yl]-6-(furan-3-yl)pyrido-[3,2-d]pyrimidine

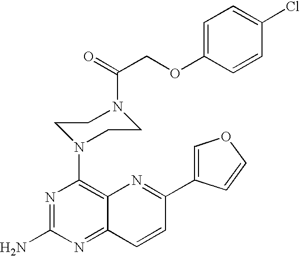

This compound was prepared according to the procedure of example 399, using 3-furanboronic acid and 2-amino-4-[4-(4-chlorophenoxyacetyl)piperazin-1-yl]-6-chloro-pyrido-[3,2-d]pyrimidine as starting materials. Yield: 41% for the title compound which was characterised as follows: MS (m/z): 465, 467 ([M+H]⁺, 100).

### EXAMPLE 403

### Synthesis of 2-amino-4-[4-(4-chlorophenoxyacetyl)piperazin-1-yl]-6-(thiophen-3-yl)pyrido-[3,2-d]pyrimidine

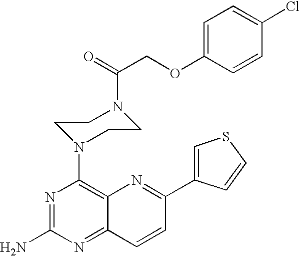

This compound was prepared according to the procedure of example 399, using 3-thiopheneboronic acid and 2-amino-4-[4-(4-chlorophenoxyacetyl)piperazin-1-yl]-6-chloro-pyrido-[3,2-d]pyrimidine as starting materials, Yield: 67% for the title compound which was characterised as follows: MS (m/z): 481, 483 ([M+H]⁺, 100).

### EXAMPLE 404

### Synthesis of 2-amino-4-[4-(4-chlorophenoxyacetyl)piperazin-1-yl]-6-(3,4-difluorophenyl)pyrido-[3,2-d]pyrimidine

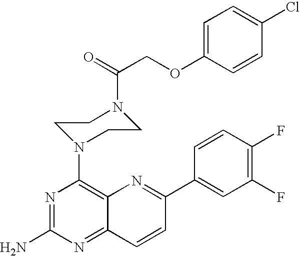

This compound was prepared according to the procedure of example 399, using 3,4-difluorophenylboronic acid and 2-amino-4-[4-(4-chlorophenoxyacetyl)piperazin-1-yl]-6-chloro-pyrido-[3,2-d]pyrimidine as starting materials. Yield: 71% for the title compound which was characterised as follows: MS (m/z): 511, 513 ([M+H]⁺, 100).

### EXAMPLE 405

### Synthesis of 2-amino-4-[4-(4-chlorophenoxyacetyl)piperazin-1-yl]-6-(4-chlorophenyl)pyrido-[3,2-d]pyrimidine

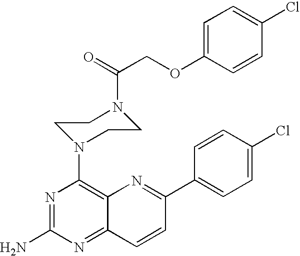

This compound was prepared according to the procedure of example 399, using 4-chlorophenylboronic acid and 2-amino-4-[4-(4-chlorophenoxyacetyl)piperazin-1-yl]-6-chloro-pyrido-[3,2-d]pyrimidine as starting materials. Yield: 53% for the title compound which was characterised as follows: MS (m/z): 509, 511 ([M+H]⁺, 100).

### EXAMPLE 406

### Synthesis of 2-amino-4-[4-(4-chlorophenoxyacetyl)piperazin-1-yl]-6-(3-chlorophenyl)pyrido-[3,2-d]pyrimidine

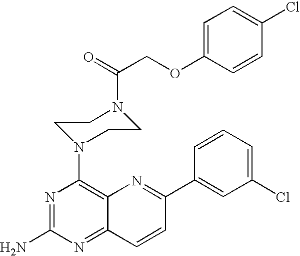

This compound was prepared according to the procedure of example 399, using 3-chlorophenylboronic acid and 2-amino-4-[4-(4-chlorophenoxyacetyl)piperazin-1-yl]-6-chloro-pyrido-[3,2-d]pyrimidine as starting materials. Yield: 79% for the title compound which was characterised as follows: MS (m/z): 509, 511 ([M+H]⁺, 100).

### EXAMPLE 407

### Synthesis of 2-amino-4-[4-(4-chlorophenoxyacetyl)piperazin-1-yl]-6-(pyridin-4-yl)pyrido-[3,2-d]pyrimidine

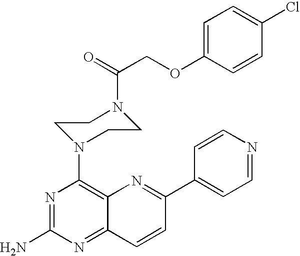

This compound was prepared according to the procedure of example 399, using 4-pyridineboronic acid and 2-amino-4-[4-(4-chlorophenoxy-acetyl)piperazin-1-yl]-6-chloro-pyrido-[3,2-d]pyrimidine as starting materials and a reaction time of 1.5 h. Yield: 79% for the title compound which was characterised as follows: MS (m/z): 476, 478 ([M+H]⁺, 100).

### EXAMPLE 408

### Synthesis of 2-amino-4-[4-(4-chlorophenoxyacetyl)piperazin-1-yl]-6-(3-chloro-4-fluorophenyl)-pyrido-[3,2-d]pyrimidine

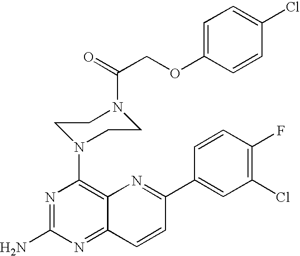

This compound was prepared according to the procedure of example 399, using 3-chloro-4-fluorophenylboronic acid and 2-amino-4-[4-(4-chlorophenoxyacetyl)piperazin-1-yl]-6-chloro-pyrido-[3,2-d]pyrimidine as starting materials. Yield: 56% for the title compound which was characterised as follows: MS (m/z): 527, 529 ([M+H]⁺, 100).

### EXAMPLE 409

### Synthesis of 2-amino-4-[4-(4-chlorophenoxyacetyl)piperazin-1-yl]-6-(pyridin-3-yl)pyrido-[3,2-d]pyrimidine

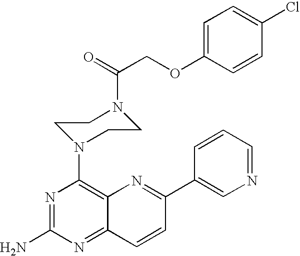

This compound was prepared according to the procedure of example 399, using 3-pyridineboronic acid and 2-amino-4-[4-(4-chlorophenoxyacetyl)piperazin-1-yl]-6-chloro-pyrido-[3,2-d]pyrimidine as starting materials. Yield: 100% for the title compound which was characterised as follows: MS (m/z): 476, 478 ([M+H]⁺, 100).

### EXAMPLE 410

### Synthesis of 2-amino-4-[4-(4-chlorophenoxyacetyl)piperazin-1-yl]-6-(2-methoxypyridin-5-yl)pyrido-[3,2-d]pyrimidine

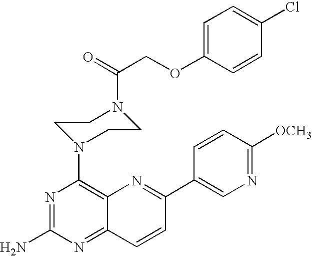

This compound was prepared according to the procedure of example 399, using 2-methoxy-5-pyridineboronic acid and 2-amino-4-[4-(4-chloro-phenoxyacetyl)piperazin-1-yl]-6-chloro-pyrido-[3,2-d]pyrimidine as starting materials. Yield: 86% for the title compound which was characterised as follows: MS (m/z): 506, 508 ([M+H]⁺, 100).

### EXAMPLE 411

### Synthesis of 2-amino-4-[4-(4-chlorophenoxyacetyl)piperazin-1-yl]-6-(3,5-dimethylisoxazol-4-yl)pyrido-[3,2-o]pyrimidine

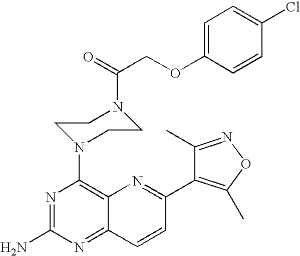

This compound was prepared according to the procedure of example 399, using 3,5-dimethylisoxazole-4-boronic acid and 2-amino-4-[4-(4-chlorophenoxyacetyl)piperazin-1-yl]-6-chloro-pyrido-[3,2-d]pyrimidine as starting materials and a reaction time of 2 h. Yield: 53% for the title compound which was characterised as follows: MS (m/z): 494, 496 ([M+H]⁺, 100).

### EXAMPLE 412

### Synthesis of 2-amino-4-[4-(4-chlorophenoxyacetyl)piperazin-1-yl]-6-(indol-5-yl)pyrido-[3,2-d]pyrimidine

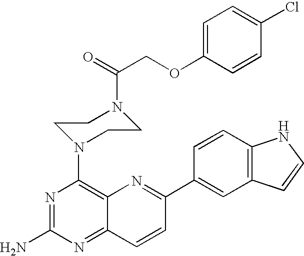

This compound was prepared according to the procedure of example 399, using 5-indolylboronic acid and 2-amino-4-[4-(4-chlorophenoxyacetyl)piperazin-1-yl]-6-chloro-pyrido-[3,2-d]pyrimidine as starting materials. Yield: 60% for the title compound which was characterised as follows: MS (m/z): 514, 516 ([M+H]⁺, 100).

### EXAMPLE 413

### Synthesis of 2-amino-4-[4-(4-chloronhenoxyacetyl)piperazin-1-yl]-6-(2-carboxythiophen-5-yl)pyrido-[3,2-d]pyrimidine

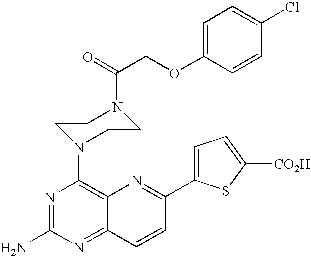

This compound was prepared according to the procedure of example 399, using 5-(dihydroxyboryl)-2-thiophenecarboxylic acid and 2-amino-4-[4-(4-chlorophenoxyacetyl)piperazin-1-yl]-6-chloro-pyrido-[3,2-d]pyrimidine as starting materials and a reaction time of 30 min. The title compound (4 mg, 8%) was isolated by precipitation and further washing with 10% ethanol in dichloromethane, and was characterised as follows: MS (m/z): 525, 527 ([M+H]⁺, 100).

### EXAMPLE 414

### Synthesis of 2-amino-4-[4-(4-chlorophenoxyacetyl)piperazin-1-yl]-6-(3-cyanophenyl)pyrido-[3,2-d]pyrimidine

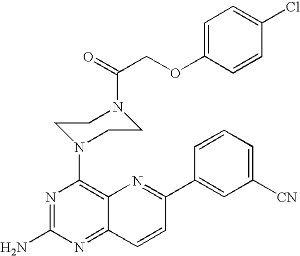

This compound was prepared according to the procedure of example 399, using 3-cyanophenylboronic acid and 2-amino-4-[4-(4-chlorophenoxy-acetyl)piperazin-1-yl]-6-chloro-pyrido-[3,2-d]pyrimidine as starting materials and a reaction time of 2 h. Yield: 89% for the title compound which was characterised as follows: MS (m/z): 500, 502 ([M+H]⁺, 100).

### EXAMPLE 415

### Synthesis of 2-amino-4-[4-(4-chlorophenoxyacetyl)piperazin-1-yl]-6-(4-hydroxyphenyl)pyrido-[3,2-d]pyrimidine

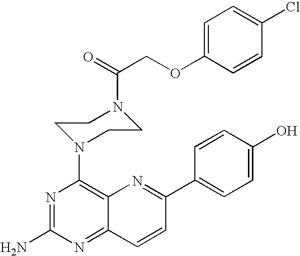

This compound was prepared according to the procedure of example 399, using 4-hydroxyphenylboronic acid and 2-amino-4-[4-(4-chlorophenoxy-acetyl)piperazin-1-yl]-6-chloro-pyrido-[3,2-d]pyrimidine as starting materials. Yield: 100% for the title compound which was characterised as follows: MS (m/z): 491, 493 ([M+H]⁺, 100).

### EXAMPLE 416

### Synthesis of 2-amino-4-[4-(4-chlorophenoxyacetyl)piperazin-1-yl]-6-(2-cyanophenyl)pyrido-[3,2-d]pyrimidine

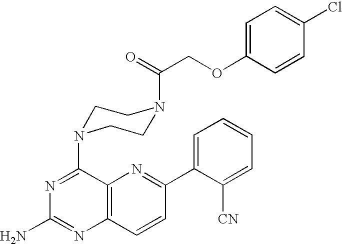

This compound was prepared according to the procedure of example 399, using 2-cyanophenylboronic acid and 2-amino-4-[4-(4-chlorophenoxyacetyl)piperazin-1-yl]-6-chloro-pyrido-[3,2-d]pyrimidine as starting materials. Yield: 49% for the title compound which was characterised as follows: MS (m/z): 500, 502 ([M+H]⁺, 100).

### EXAMPLE 417

### Synthesis of 2-amino-4-[4-(4-chlorophenoxyacetyl)piperazin-1-yl]-6-(4-(methanesulfonyl)-phenyl)pyrido-[3,2-d]pyrimidine

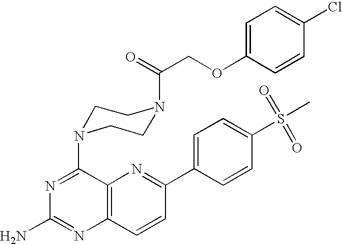

This compound was prepared according to the procedure of example 399, using 4-(methanesulfonyl)phenylboronic acid and 2-amino-4-[4-(4-chlorophenoxyacetyl)piperazin-1-yl]-6-chloro-pyrido-[3,2-d]pyrimidine as starting materials. Yield: 62% for the title compound which was characterised as follows: MS (m/z): 553, 555 ([M+H]⁺, 100).

### EXAMPLE 418

### Synthesis of 2-amino-4-[4-(4-chlorophenoxyacetyl)piperazin-1-yl]-6-(3-methoxyphenyl)pyrido-[3,2-d]pyrimidine

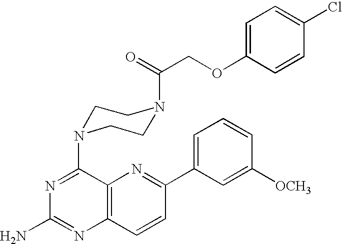

This compound was prepared according to the procedure of example 399, using 3-methoxyphenylboronic acid and 2-amino-4-[4-(4-chlorophenoxyacetyl)piperazin-1-yl]-6-chloro-pyrido-[3,2-d]pyrimidine as starting materials. Yield: 72% for the title compound which was characterised as follows: MS (m/z): 505, 507 ([M+H]⁺, 100).

### EXAMPLE 419

### Synthesis of 2-amino-4-[4-(4-chlorophenoxyacetyl)piperazin-1-yl]-6-(3-aminophenyl)pyrido-[3,2-d]pyrimidine

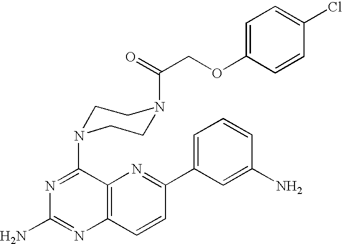

This compound was prepared according to the procedure of example 399, using 3-aminophenylboronic acid and 2-amino-4-[4-(4-chlorophenoxy-acetyl)piperazin-1-yl]-6-chloro-pyrido-[3,2-d]pyrimidine as starting materials and a reaction time of 1.5 h. Yield: 80% for the title compound which was characterised as follows: MS (m/z): 490, 492 ([M+H]⁺, 100).

### EXAMPLE 420

### Synthesis of 2-amino-4-[4-(4-chlorophenoxyacetyl)piperazin-1-yl]-6-(4-fluoro-3-methylphenyl)-pyrido-[3,2-d]pyrimidine

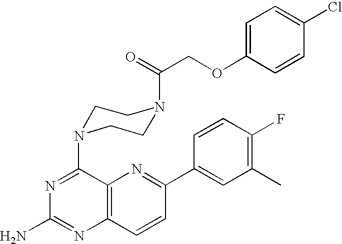

This compound was prepared according to the procedure of example 399, using 4-fluoro-3-methylphenylboronic acid and 2-amino-4-[4-(4-chlorophenoxyacetyl)piperazin-1-yl]-6-chloro-pyrido-[3,2-d]pyrimidine as starting materials. Yield: 91% for the title compound which was characterised as follows: MS (m/z): 507, 509 ([M+H]⁺, 100).

### EXAMPLE 421

### Synthesis of 2-amino-4-[4-(4-chlorophenoxyacetyl)piperazin-1-yl]-6-phenylpyrido-[3,2-d]pyrimidine

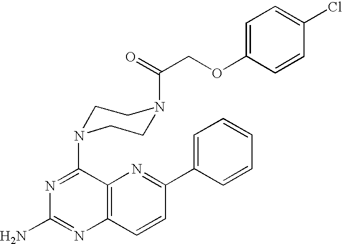

This compound was prepared according to the procedure of example 399, using phenylboronic acid and 2-amino-4-[4-(4-chlorophenoxy-acetyl)piperazin-1-yl]-6-chloro-pyrido-[3,2-d]pyrimidine as starting materials. Yield: 42% for the title compound which was characterised as follows: MS (m/z): 475, 477 ([M+H]⁺, 100).

### EXAMPLE 422

### Synthesis of 2-amino-4-[4-(4-chlorophenoxyacetyl)piperazin-1-yl]-6-(2-methoxyphenyl)pyrido-[3,2-d]pyrimidine

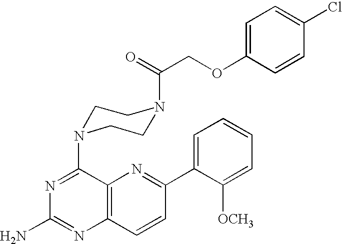

This compound was prepared according to the procedure of example 399, using 2-methoxyphenylboronic acid and 2-amino-4-[4-(4-chlorophenoxy-acetyl)piperazin-1-yl]-6-chloro-pyrido-[3,2-d]pyrimidine as starting materials. Yield: 88% for the title compound which was characterised as follows: MS (m/z): 505, 507 ([M+H]⁺, 100).

### EXAMPLE 423

### Synthesis of 2-amino-4-[4-(4-chlorophenoxyacetyl)piperazin-1-yl]-6-(2,4-difluorophenyl)pyrido-[3,2-d]pyrimidine

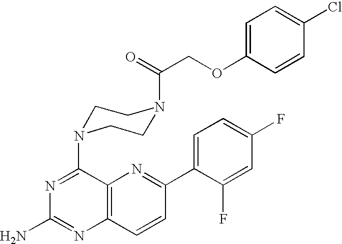

This compound was prepared according to the procedure of example 399, using 2,4-difluorophenylboronic acid and 2-amino-4-[4-(4-chlorophenoxyacetyl)piperazin-1-yl]-6-chloro-pyrido-[3,2-d]pyrimidine as starting materials. Yield: 85% for the title compound which was characterised as follows: MS (m/z): 511, 513 ([M+H]⁺, 100).

### EXAMPLE 424

### Synthesis of 2-amino-4-[4-(4-chlorophenoxyacetyl)piperazin-1-yl]-6-(2-fluorophenyl)pyrido-[3,2-d]pyrimidine

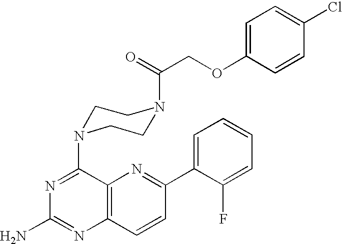

This compound was prepared according to the procedure of example 399, using 2-fluorophenylboronic acid and 2-amino-4-[4-(4-chlorophenoxyacetyl)piperazin-1-yl]-6-chloro-pyrido-[3,2-d]pyrimidine as starting materials. Yield: 98% for the title compound which was characterised as follows: MS (m/z): 493, 495 ([M+H]⁺, 100).

### EXAMPLE 425

### Synthesis of 2-amino-4-[4-(4-chlorophenoxyacetyl)piperazin-1-yl]-6-(2,3-dichlorophenyl)-pyrido-[3,2-d]pyrimidine

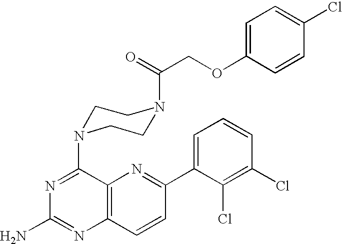

This compound was prepared according to the procedure of example 399, using 2,3-dichlorophenylboronic acid and 2-amino-4-[4-(4-chlorophenoxyacetyl)piperazin-1-yl]-6-chloro-pyrido-[3,2-d]pyrimidine as starting materials. Yield: 100% for the title compound which was characterised as follows: MS (m/z): 543, 545 ([M+H]⁺, 100).

### EXAMPLE 426

### Synthesis of 2-amino-4-[4-(4-chlorophenoxyacetyl)piperazin-1-yl]-6-(4-methoxyphenyl)pyrido-[3,2-d]pyrimidine

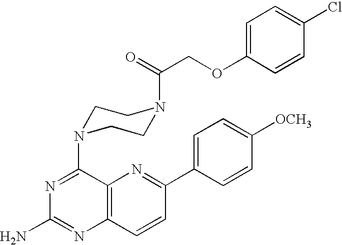

This compound was prepared according to the procedure of example 399, using 4-methoxyphenylboronic acid and 2-amino-4-[4-(4-chlorophenoxyacetyl)piperazin-1-yl]-6-chloro-pyrido-[3,2-d]pyrimidine as starting materials. Yield: 78% for the title compound which was characterised as follows: MS (m/z): 505, 507 ([M+H]⁺, 100).

### EXAMPLE 427

### Synthesis of 2-amino-4-[4-(4-chlorophenoxyacetyl)piperazin-1-yl]-6-(2,4-dichlorophenyl)-pyrido-[3,2-d]pyrimidine

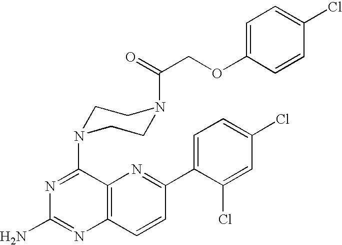

This compound was prepared according to the procedure of example 399, using 2,4-dichlorophenylboronic acid (49 mg, 0.26 mmol), 2-amino-4-[4-(4-chlorophenoxyacetyl)piperazin-1-yl]-6-chloro-pyrido-[3,2-d]pyrimidine and tetrakis (triphenylphosphine)palladium(0) (14 mg) as starting materials and a reaction time of 2 h. Yield: 10% for the title compound which was characterised as follows: MS (m/z): 543, 545 ([M+H]⁺, 100).

### EXAMPLE 428

### Synthesis of 2-amino-4-[4-(4-chlorophenoxyacetyl)piperazin-1-yl]-6-(2,6-difluorophenyl)pyrido-[3,2-d]pyrimidine

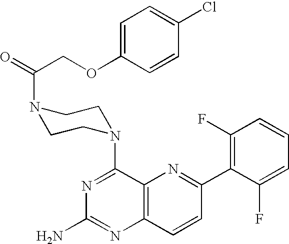

This compound was prepared according to the procedure of example 399, using 2,6-difluorophenylboronic acid (54 mg, 0.34 mmol), 2-amino-4-[4-(4-chlorophenoxyacetyl)piperazin-1-yl]-6-chloro-pyrido-[3,2-d]pyrimidine and tetrakis (triphenylphosphine)palladium(0) (16 mg) as starting materials and a reaction time of 3.5 h. Yield: 100% for the title compound which was characterised as follows: MS (m/z): 511, 513 ([M+H]⁺, 100).

### EXAMPLE 429

### Synthesis of 2-amino-4-[4-(4-chlorophenoxyacetyl)piperazin-1-yl]-6-(2,5-dichlorophenyl)-pyrido-[3,2-d]pyrimidine

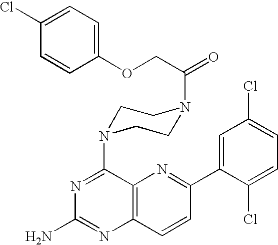

This compound was prepared according to the procedure of example 399, using 2,5-dichlorophenylboronic acid and 2-amino-4-[4-(4-chlorophenoxyacetyl)piperazin-1-yl]-6-chloro-pyrido-[3,2-d]pyrimidine as starting materials. Yield: 61% for the title compound which was characterised as follows: MS (m/z): 543, 545 ([M+H]⁺, 100).

### EXAMPLE 430

### Synthesis of 2-amino-4-[4-(4-chlorophenoxyacetyl)piperazin-1-yl]-6-(2-chlorophenyl)pyrido-[3,2-d]pyrimidine

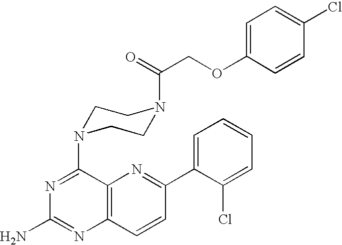

This compound was prepared according to the procedure of example 399, using 2-chlorophenylboronic acid (29 mg, 0.18 mmol) and 2-amino-4-[4-(4-chlorophenoxyacetyl)piperazin-1-yl]-6-chloro-pyrido-[3,2-d]pyrimidine as starting materials. Yield: 73% for the title compound which was characterised as follows: MS (m/z): 509, 511 ([M+H]⁺, 100).

### EXAMPLE 431

### Synthesis of 2-amino-4-[4-(4-chlorophenoxyacetyl)piperazin-1-yl]-6-(5-chloro-2-fluorophenyl)-pyrido-[3,2-d]pyrimidine

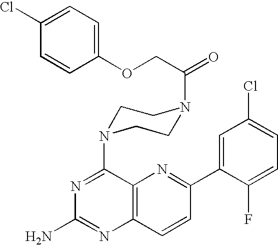

This compound was prepared according to the procedure of example 399, using 5-chloro-2-fluorophenylboronic acid and 2-amino-4-[4-(4-chloro-phenoxyacetyl)piperazin-1-yl]-6-chloro-pyrido-[3,2-d]pyrimidine as starting materials. Yield: 100% for the title compound which was characterised as follows: MS (m/z): 527, 529 ([M+H]⁺, 100).

### EXAMPLE 432

### Synthesis of 2-amino-4-[4-(4-chlorophenoxyacetyl)piperazin-1-yl]-6-(3,4,5-trifluorophenyl)-pyrido-[3,2-d]pyrimidine

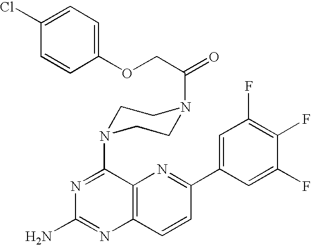

This compound was prepared according to the procedure of example 399, using 3,4,5-trifluorophenylboronic acid and 2-amino-4-[4-(4-chlorophenoxy-acetyl)piperazin-1-yl]-6-chloro-pyrido-[3,2-d]pyrimidine as starting materials. Yield: 100% for the title compound which was characterised as follows: MS (m/z): 529, 531 ([M+H]⁺, 100).

### EXAMPLE 433

### Synthesis of 2-amino-4-[4-(4-chlorophenoxyacetyl)piperazin-1-yl]-6-(2,6-dimethylphenyl)-pyrido-[3,2-d]pyrimidine

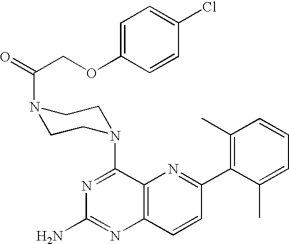

This compound was prepared according to the procedure of example 399, using 2,6-dimethylphenylboronic acid (41 mg, 0.28 mmol), 2-amino-4-[4-(4-chlorophenoxyacetyl)piperazin-1-yl]-6-chloro-pyrido-[3,2-d]pyrimidine and tetrakis (triphenylphosphine)palladium(0) (13 mg) as starting materials and a reaction time of 3 h. Yield: 87% for the title compound which was characterised as follows: MS (m/z): 503, 505 ([M+H]⁺, 100).

### EXAMPLE 434

### Synthesis of 2-amino-4-{4-[N-(tert-butoxycarbonyl)-glycyl-piperazin-1-yl}-6-(4-fluorophenyl)-pyrido[3,2-d]pyrimidine

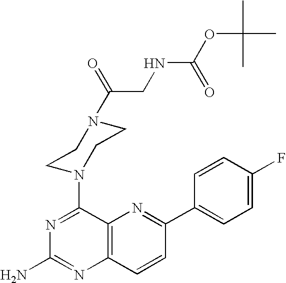

A solution of 2-amino-4-(N-piperazin-1-yl)-6-(4-fluorophenyl)-pyrido[3,2-d]pyrimidine (114 mg, 0.35 mmol), N-(tert-butoxycarbonyl)glycine (137 mg, 0.78 mmol), DIPEA (162 μl, 0.97 mmol) and TBTU (226 mg, 0.70 mmol) in dry dioxane (10 ml) was stirred under an N₂ atmosphere for 2 hours at room temperature. The reaction mixture was partitioned between dichloromethane and water and the aqueous layer was extracted three times with dichloromethane. The combined organic layers were dried over magnesium sulfate, filtered and concentrated under reduced pressure. Purification by chromatography on a column of silica, eluting with 10% methanol in dichloromethane yielded the title compound (108 mg, 64%) which was characterised as follows: MS (m/z): 482 ([M+H]⁺, 100).

### EXAMPLE 435

### Synthesis of 2-amino-4-[4-(4-chlorophenoxyacetyl)piperazin-1-yl]-6-[N-3-(methanesulfonamidophenyl)]-pyrido-[3,2-d]pyrimidine

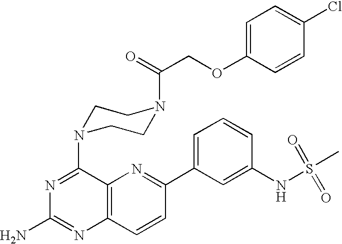

This compound was prepared according to the procedure of example 399, using N-3-methanesulfonamidephenylboronic acid and 2-amino-4-[4-(4-chloro-phenoxyacetyl)piperazin-1-yl]-6-chloro-pyrido-[3,2-d]pyrimidine as starting materials. Yield: 73% for the title compound which was characterised as follows: MS (m/z): 568, 570 ([M+H]⁺, 100).

### EXAMPLE 436

### Synthesis of 2-amino-4-[4-(2-hydroxyacetyl)piperazin-1-yl]-6-chloro-pyrido[3,2-d]pyrimidine

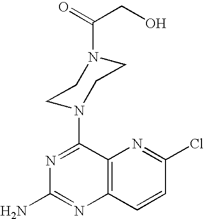

A solution of 2-amino-4-(N-piperazin-1-yl)-6-chloro-pyrido[3,2-d]pyrimidine (48 mg, 0.18 mmol), glycolic acid (18 mg, 0.23 mmol), DIPEA (85 μl, 0.49 mmol) and TBTU (100 mg, 0.31 mmol) in dry DMF (2 ml) was stirred under an N₂ atmosphere for 5 h at room temperature. The reaction mixture was applied directly onto a column of silica gel packed in 4% methanol in dichloromethane. Elution with the same solvent mixture yielded the title compound (17 mg, 29%) which was characterised as follows:

MS (m/z): 323, 325 ([M+H]⁺, 100).

### EXAMPLE 437

### Synthesis of 2-amino-4-[4-(2-hydroxyacetyl)piperazin-1-yl]-6-(4-fluorophenyl)-pyrido[3,2-d]pyrimidine

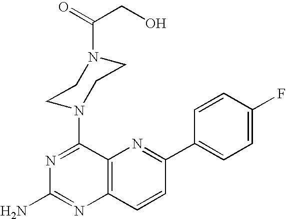

This compound was prepared according to the procedure of example 399, using 4-fluorophenylboronic acid and 2-amino-4-[4-(2-hydroxyacetyl)piperazin-1-yl]-6-chloro-pyrido[3,2-d]pyrimidine as starting materials and chromatography in 10% methanol in dichloromethane for purification. Yield: 67% for the title compound which was characterised as follows: MS (m/z): 383 ([M+H]⁺, 100).

### EXAMPLE 438

### Synthesis of 2-amino-4-[4-(4-chlorophenoxyacetyl)piperazin-1-yl]-6-(2,6-dichlorophenyl)-pyrido-[3,2-d]pyrimidine

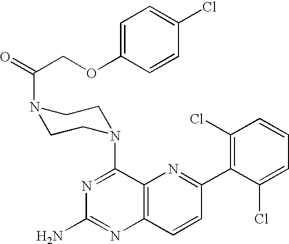

This compound was prepared according to the procedure of example 399, using 2,6-dichlorophenylboronic acid (18 mg, 92 μmol), 2-amino-4-[4-(4-chlorophenoxyacetyl)piperazin-1-yl]-6-chloro-pyrido-[3,2-d]pyrimidine and tetrakis (triphenylphosphine)palladium(0) (12 mg) as starting materials and a reaction time of 4 hours. Yield: 44% for the title compound which was characterised as follows: MS (m/z): 543, 545 ([M+H]⁺, 100).

### EXAMPLE 439

### Synthesis of 2-amino-4-[4-(4-chlorophenoxyacetyl)piperazin-1-yl]-6-(4-trifluoromethoxyphenyl)-pyrido-[3,2-d]pyrimidine

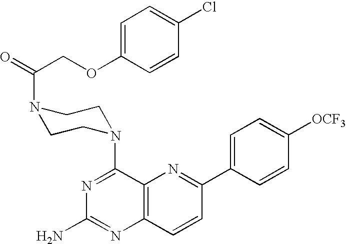

This compound was prepared according to the procedure of example 399, using 4-trifluoromethoxyphenylboronic acid and 2-amino-4-[4-(4-chlorophenoxy-acetyl)piperazin-1-yl]-6-chloro-pyrido-[3,2-d]pyrimidine as starting materials. Yield: 100% for the title compound which was characterised as follows: MS (m/z): 559, 561 ([M+H]⁺, 100).

### EXAMPLE 440

### Synthesis of 2-amino-4-[4-(4-chlorophenoxyacetyl)piperazin-1-yl]-6-(2,5-difluorophenyl)pyrido-[3,2-d]pyrimidine

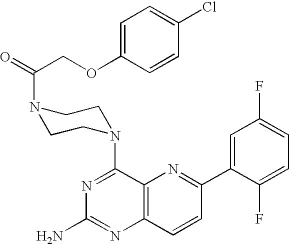

This compound was prepared according to the procedure of example 399, using 2,5-difluorophenylboronic acid (44 mg, 0.28 mmol), 2-amino-4-[4-(4-chlorophenoxyacetyl)piperazin-1-yl]-6-chloro-pyrido-[3,2-d]pyrimidine and tetrakis (triphenylphosphine)palladium(0) (14 mg, 12 μmol) as starting and a reaction time of 2.5 h. Yield: 100% for the title compound which was characterised as follows: MS (m/z): 511, 513 ([M+H]⁺, 100).

### EXAMPLE 441

### Synthesis of 2-amino-4-[4-(4-chlorophenoxyacetylpiperazin-1-yl]-6-[4-(hydroxymethyl)phenyl]pyrido-[3,2-d]pyrimidine

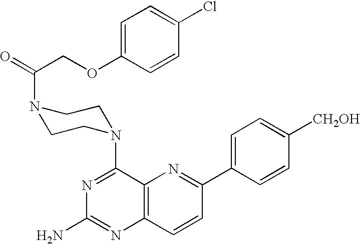

This compound was prepared according to the procedure of example 399, using 4-(hydroxymethyl)phenylboronic acid and 2-amino-4-[4-(4-chlorophenoxy-acetyl)piperazin-1-yl]-6-chloro-pyrido-[3,2-d]pyrimidine as starting materials. Yield: 100% for the title compound which was characterised as follows: MS (m/z): 505, 507 ([M+H]⁺, 100).

### EXAMPLE 442

### Synthesis of 2-amino-4-[4-(4-chlorophenoxyacetyl)piperazin-1-yl]-6-(2-chloro-6-fluorophenyl)-pyrido-[3,2-d]pyrimidine

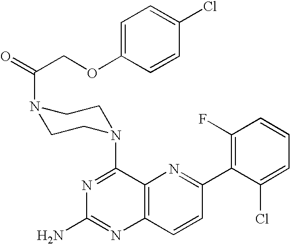

This compound was prepared according to the procedure of example 399, using 2-chloro-6-fluorophenylboronic acid and 2-amino-4-[4-(4-chlorophenoxy-acetyl)piperazin-1-yl]-6-chloro-pyrido-[3,2-d]pyrimidine as starting materials and a reaction time of 2 hours. Yield: 52% for the title compound which was characterised as follows: MS (m/z): 527, 529 ([M+H]⁺, 100).

### EXAMPLE 443

### Synthesis of 2-amino-4-[4-(4-chlorophenoxyacetyl)piperazin-1-yl]-6-[4-(methylaminocarbonyl)phenyl]-pyrido-[3,2-d]pyrimidine

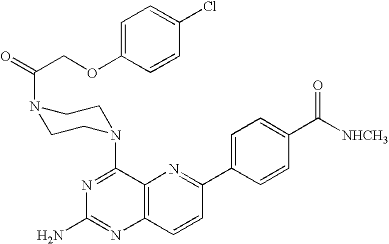

This compound was prepared according to the procedure of example 399, using 4-(N-methylaminocarbonyl)phenyl boronic acid and 2-amino-4-[4-(4-chloro-phenoxyacetyl)piperazin-1-yl]-6-chloro-pyrido-[3,2-d]pyrimidine as starting materials. Yield: 85% for the title compound which was characterised as follows: MS (m/z): 532, 534 ([M+H]⁺, 100).

### EXAMPLE 444

### Synthesis of 2-amino-4-[4-(aminoacetyl)-piperazin-1-yl]-6-(4-fluorophenyl)-pyrido[3,2-d]pyrimidine

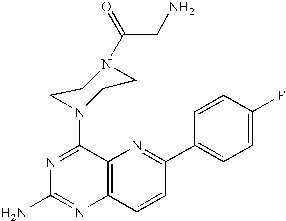

A solution of 2-amino-4-{4-[N-(tert-butoxycarbonyl)-glycyl]-piperazin-1-yl}-6-(4-fluorophenyl)-pyrido[3,2-d]pyrimidine (60 mg, 0.12 mmol) in dichloromethane (5 ml) was cooled to 0° C. Trifluoroacetic acid (2.2 ml, 28.3 mmol) was added slowly via a syringe. After 5 minutes, the ice bath was removed and the reaction mixture was stirred for a further 60 minutes, whereupon it was diluted with dichloromethane (20 ml) and treated with saturated sodium hydrogen carbonate solution until basic pH of the aqueous layer was reached. The layers were separated and the aqueous layer was extracted three times with dichloromethane. The combined organic layers are dried over magnesium sulfate, filtered and concentrated under reduced pressure. Purification by chromatography on a plate of silica, eluting with 20% methanol in dichloromethane yielded the title compound (11 mg, 24%) which was characterised as follows: MS (m/z): 382 ([M+H]⁺, 100).

### EXAMPLE 445

### Synthesis of 2-amino-4-[4-(4-chlorophenoxyacetyl)piperazin-1-yl]-6-(4-methylphenyl)pyrido-[3,2-o]pyrimidine

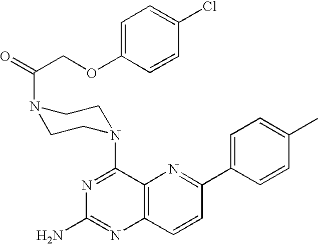

This compound was prepared according to the procedure of example 399, using p-tolylboronic acid and 2-amino-4-[4-(4-chlorophenoxyacetyl)piperazin-1-yl]-6-chloro-pyrido-[3,2-d]pyrimidine as starting materials and a reaction time of 2 hours. Yield: 98% for the title compound which was characterised as follows: MS (m/z): 489, 491 ([M+H]⁺, 100).

### EXAMPLE 446

### Synthesis of 2-amino-4-[4-(4-chlorophenoxyacetyl)piperazin-1-yl]-6-(4-acetylphenyl)pyrido-[3,2-o]pyrimidine

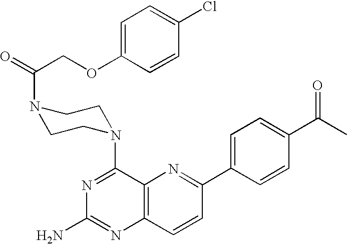

This compound was prepared according to the procedure of example 399, using 4-acetylphenylboronic acid and 2-amino-4-[4-(4-chlorophenoxyacetyl)piperazin-1-yl]-6-chloro-pyrido-[3,2-d]pyrimidine as starting materials. Yield: 80% for the title compound which was characterised as follows: MS (m/z): 517, 519 ([M+H]⁺, 100).

### EXAMPLE 447

### Synthesis of 2-amino-4-[4-(4-chlorophenoxyacetyl)piperazin-1-yl]-6-[4-(aminomethyl)phenyl]pyrido-[3,2-d]pyrimidine

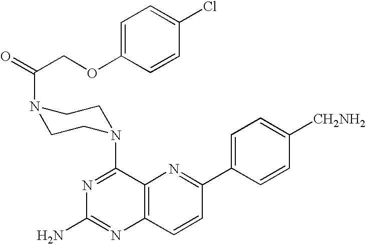

This compound was prepared according to the procedure of example 399, using 4-aminomethylphenylboronic acid hydrochloride (24 mg, 0.13 mmol), 2-amino-4-[4-(4-chlorophenoxyacetyl)piperazin-1-yl]-6-chloro-pyrido-[3,2-d]-pyrimidine and tetrakis(triphenylphosphine)palladium(0) (19 mg, 17 μmol) as starting materials. After 1 hour, one more aliquot of boronic acid derivative and palladium catalyst were added and the reaction was allowed to proceed for a further 60 minutes. Yield: 57% for the title compound which was characterised as follows: MS (m/z): 504, 506 ([M+H]⁺, 100).

### EXAMPLE 448

### Synthesis of 2-amino-4-[4-(4-chlorophenoxyacetyl)piperazin-1-yl]-6-[4-(cyclopropylaminocarbonyl)phenyl]-pyrido-[3,2-d]pyrimidine

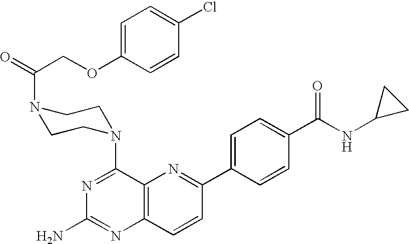

This compound was prepared according to the procedure of example 399, using 4-(cyclopropylaminocarbonyl)phenylboronic acid and 2-amino-4-[4-(4-chloro-phenoxyacetyl)piperazin-1-yl]-6-chloro-pyrido-[3,2-d]pyrimidine as starting materials and purification by chromatography in 8% methanol in dichloromethane. Yield: 71% for the title compound which was characterised as follows: MS (m/z): 558, 560 ([M+H]⁺, 100).

### EXAMPLE 449

### Synthesis of 2-amino-4-[4-(4-chlorophenoxyacetyl)piperazin-1-yl]-6-[4-(acetamido)phenyl]-pyrido-[3,2-d]pyrimidine

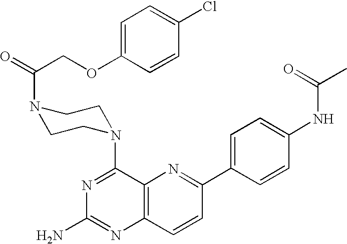

This compound was prepared according to the procedure of example 399, using 4-(acetamido)phenylboronic acid and 2-amino-4-[4-(4-chlorophenoxyacetyl)piperazin-1-yl]-6-chloro-pyrido-[3,2-d]pyrimidine as starting materials and purification by chromatography in 8-10% methanol in dichloromethane. Yield: 48% for the title compound which was characterised as follows: MS (m/z): 532, 534 ([M+H]⁺, 100).

### EXAMPLE 450

### Synthesis of 2-amino-4-[4-(N-ethylcarbamoyl)-piperazin-1-yl]-6-(4-fluorophenyl)-pyrido[3,2-d]pyrimidine

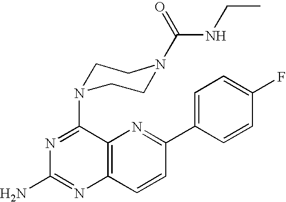

Ethyl isocyanate (12 μl, 150 μmol) was added to a suspension of 2-amino-4-(N-piperazin-1-yl)-6-(4-fluorophenyl)-pyrido[3,2-d]pyrimidine (43 mg, 130 μmol) in a mixture of dichloromethane (2 ml) and DMF (2 ml). After stirring for 30 minutes at room temperature (the reaction mixture then becomes clear), the whole mixture was applied directly onto a column of silica gel packed in 10% methanol in dichloromethane. Elution with the same solvent mixture yielded the title compound (16 mg, 31%) which was characterised as follows: MS (m/z): 396 ([M+H]⁺, 100).

### EXAMPLE 451

### Synthesis of 2-amino-4-[4-(N-butylcarbamoyl)-piperazin-1-yl]-6-(4-fluorophenyl)-pyrido[3,2-d]pyrimidine

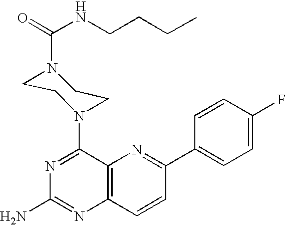

This compound was prepared according to the procedure of example 450, using n-butyl isocyanate and 2-amino-4-(N-piperazin-1-yl)-6-(4-fluorophenyl)-pyrido[3,2-d]pyrimidine as starting materials. Yield: 26% for the title compound which was characterised as follows: MS (m/z): 424 ([M+H]⁺, 100).

### EXAMPLE 452

### Synthesis of 2-amino-4-[4-(N-methylcarbamoyl)-piperazin-1-yl]-6-(4-fluorophenyl)-pyrido[3,2-d]pyrimidine

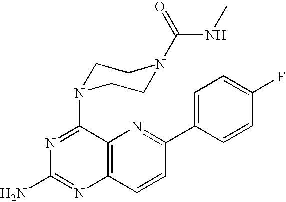

This compound was prepared according to the procedure of example 450, using methyl isocyanate and 2-amino-4-(N-piperazin-1-yl)-6-(4-fluorophenyl)-pyrido[3,2-d]pyrimidine as starting materials. Yield: 24% for the title compound which was characterised as follows: MS (m/z): 382 ([M+H]⁺, 100).

### EXAMPLE 453

### Synthesis of 2-amino-4-{4-[N-(1-adamantylcarbamoyl)]-piperazin-1-yl}-6-(4-fluorophenyl)-pyrido[3,2-d]pyrimidine

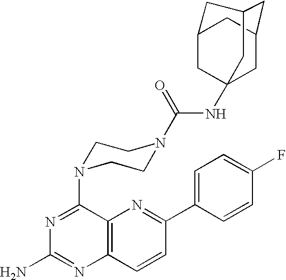

This compound was prepared according to the procedure of example 450, using 1-adamantyl isocyanate and 2-amino-4-(N-piperazin-1-yl)-6-(4-fluorophenyl)-pyrido[3,2-d]pyrimidine as starting materials. Yield: 35% for the title compound which was characterised as follows: MS (m/z): 502 ([M+H]⁺, 100).

### EXAMPLE 454

### Synthesis of 2-amino-4-[4-(N-cyclopentylcarbamoyl)-piperazin-1-yl]-6-(4-fluorophenyl)-pyrido[3,2-d]pyrimidine

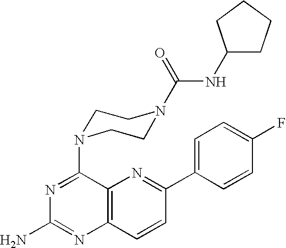

This compound was prepared according to the procedure of example 450, using cyclopentyl isocyanate and 2-amino-4-(N-piperazin-1-yl)-6-(4-fluorophenyl)-pyrido[3,2-d]pyrimidine as starting materials, HPLC grade acetonitrile as solvent and a reaction time of 2.5 hours. After 60 minutes following the start of the reaction, two additional equivalents of cyclopentyl isocyanate were added, followed by further two equivalents of cyclopentyl isocyanate and dry dioxane (1 ml) after another 60 minutes. Yield: 27% for the title compound which was characterised as follows: MS (m/z): 436 ([M+H]⁺, 100).

### EXAMPLE 455

### Synthesis of 2-amino-4-{4-[N-(4-chlorophenyl)carbamoyl]-piperazin-1yl}-6-(4-fluorophenyl)-pyrido[3,2-d]pyrimidine

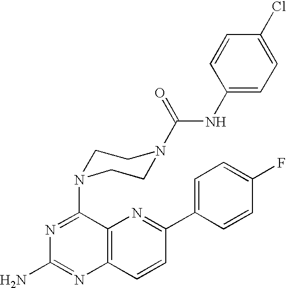

This compound was prepared according to the procedure of example 450, using 4-chlorophenyl isocyanate and 2-amino-4-(N-piperazin-1-yl)-6-(4-fluorophenyl)-pyrido[3,2-d]pyrimidine as starting materials and dry DMF (2 ml) as solvent. Yield: 20% for the title compound which was characterised as follows: MS (m/z): 478, 480 ([M+H]⁺, 100).

### EXAMPLE 456

### Synthesis of 2-amino-4-[4-(2-phenoxyethyl)-piperazin-1-yl]-6-(4-fluorophenyl)-pyrido[3,2-o]pyrimidine

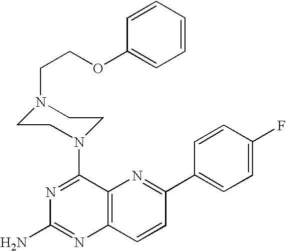

A suspension of 2-amino-4-oxo-6-(4-fluorophenyl)-pyrido-[3,2-d]-pyrimidine (51 mg, 0.19 mmol) and 1,8-diazabicyclo[5.4.0]undec-7-ene (DBU; 55 μl, 0.36 mmol) in dry DMF (10 ml) was homogenized by brief sonication and stirred under an N₂ atmosphere for 15 minutes. (Benzotriazol-1-yloxy)tris(dimethylamino)phosphonium hexafluoro-phosphate (BOP; 119 mg, 0.27 mmol) and 1-(2-phenoxyethyl)piperazine (90 mg, 0.43 mmol) were added and the reaction mixture was stirred for 18 hours, whereupon it was partitioned between dichloromethane (50 ml) and water (50 ml). The organic layer was concentrated under reduced pressure. Purification by chromatography on a column of silica, eluting with 10% methanol in dichloromethane yielded the title compound (84 mg, ˜100%; lyophilization was necessary to completely remove DMF) which was characterised as follows: MS (m/z): 445 ([M+H]⁺, 100).

### EXAMPLE 457

### Synthesis of 2-amino-4-[1-(tert-butoxycarbonyl)piperid-3-ylamino]-6-(4-fluorophenyl)-pyrido[3,2-d]pyrimidine

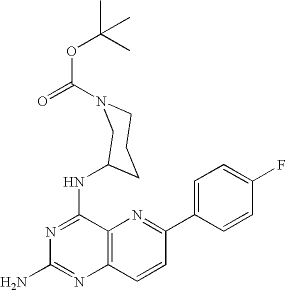

A suspension of 2-amino-4-oxo-6-(4-fluorophenyl)-pyrido-[3,2-d]-pyrimidine (21 mg, 80 μmol) and 1,8-diazabicyclo[5.4.0]undec-7-ene (DBU; 48 μl, 0.31 mmol) in HPLC grade acetonitrile (5 ml) was homogenized by brief sonication and stirred under an N₂ atmosphere for 15 minutes. (Benzotriazol-1-yloxy)tris(dimethylamino)phosphonium hexafluorophosphate (BOP; 59 mg, 0.13 mmol) and 3-amino-1-Boc-piperidine hydrochloride (43 mg, 0.18 mmol) were added and the reaction mixture was stirred for 4 hours, whereupon it was partitioned between dichloromethane (25 ml) and water (25 ml). The aqueous layer was extracted two times with dichloromethane and the combined organic layers were dried over magnesium sulfate and concentrated under reduced pressure. Purification by chromatography on a column of silica, eluting with 10% methanol in dichloromethane yielded the title compound (12 mg, 34%) which was characterised as follows: MS (m/z): 439 ([M+H]⁺, 100).

### EXAMPLE 458

### Synthesis of 2-amino-4-[4-(benzyloxycarbonyl)-piperazin-1-yl]-6-(4-fluorophenyl)-pyrido[3,2-d]pyrimidine

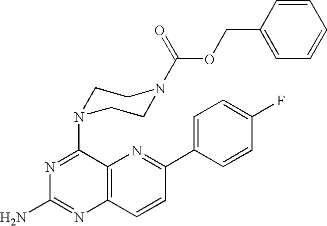

This compound was prepared according to the procedure of example 457, using 1-(benzyloxycarbonyl)piperazine and 2-amino-4-oxo-6-(4-fluorophenyl)-pyrido-[3,2-d]-pyrimidine as starting materials, a reaction time of 1.5 hour and the following work-up: the reaction mixture was partitioned between dichloromethane (25 ml) and water (25 ml). The aqueous layer was extracted three times with dichloromethane and the combined organic layers were dried over magnesium sulfate and concentrated under reduced pressure. Purification by chromatography on a column of silica, eluting with 10% methanol in dichloromethane yielded the title compound (60 mg, 82%) which was characterised as follows: MS (m/z): 459 ([M+H]⁺, 100).

### EXAMPLE 459

### Synthesis of 2-amino-4-{4-[2-(phenyl)ethylcarbamylmethyl]-piperazin-1-yl}-6-(4-fluorophenyl)-pyrido[3,2-o]pyrimidine

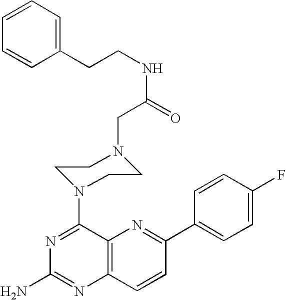

This compound was prepared according to the procedure of example 457, using 2-(piperazin-1-yl)-acetic acid N-(2-phenylethyl)amide and 2-amino-4-oxo-6-(4-fluorophenyl)-pyrido-[3,2-d]-pyrimidine as starting materials and dry dioxane as solvent. Yield: 95% for the title compound which was characterised as follows: MS (m/z): 486 ([M+H]⁺, 100).

### EXAMPLE 460

### Synthesis of 2-amino-4-[4-(4-chlorophenyl)-piperazin-1-yl]-6-(4-fluorophenyl)-pyrido[3,2-d]pyrimidine

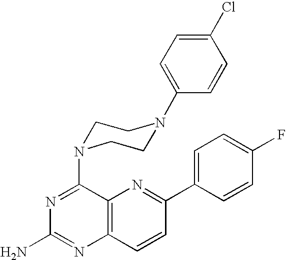

This compound was prepared according to the procedure of example 457, using 1-(4-chlorophenyl)piperazine dihydrochloride, 2-amino-4-oxo-6-(4-fluorophenyl)-pyrido-[3,2-d]-pyrimidine and DBU (212 μl, 1.39 mmol) as starting materials and a reaction time of 4 hours. Yield: 34% for the title compound which was characterised as follows: MS (m/z): 435, 437 ([M+H]⁺, 100).

### EXAMPLE 461

### Synthesis of 2-amino-4-[1-(tert-butoxycarbonyl)piperid-4-ylamino]-6-(4-fluorophenyl)-pyrido[3,2-d]pyrimidine

This compound was prepared according to the procedure of example 457, using 4-amino-1-Boc-piperidine and 2-amino-4-oxo-6-(4-fluorophenyl)-pyrido-[3,2-d]pyrimidine as starting materials and dry dioxane as solvent. Yield: 43% for the title compound which was characterised as follows: MS (m/z): 439 ([M+H]⁺, 100).

### EXAMPLE 462

### Synthesis of 2-amino-4-[4-(methoxyacetyl)piperazin-1-yl]-6-(4-fluorophenyl)-pyrido[3,2-d]pyrimidine

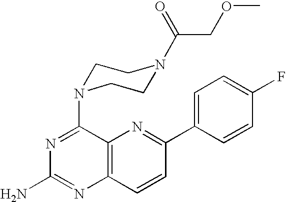

DIPEA (32 μl, 0.19 mmol) was added to a nitrogen purged solution of 2-amino-4-(N-piperazin-1-yl)-6-(4-fluorophenyl)-pyrido[3,2-d]pyrimidine (53 mg, 0.16 mmol) in dry DMF (2 ml). Methoxyacetyl chloride (15 μl, 0.16 mmol) was dissolved in 0.5 ml of dry DMF and this solution was added dropwise to the above mixture. After stirring for 30 minutes at room temperature, the reaction mixture was applied directly onto a column of silica gel packed in 10% methanol in dichloromethane. Elution with the same solvent mixture yielded the title compound (31 mg, 49%) which was characterised as follows: MS (m/z): 397 ([M+H]⁺, 100).

### EXAMPLE 463

### Synthesis of 2-amino-4-[4-(diethylcarbamoyl)piperazin-1-yl]-6-(4-fluorophenyl)-pyrido[3,2-d]pyrimidine

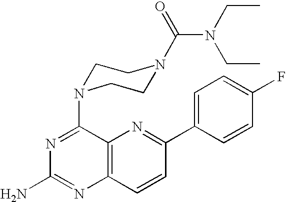

A suspension of 2-amino-4-(N-piperazin-1-yl)-6-(4-fluorophenyl)-pyrido[3,2-d]pyrimidine (46 mg, 0.14 mmol) and DIPEA (140 μl, 0.83 mmol) in dry dioxane (5 ml) was brought under an N₂ atmosphere and homogenized by brief sonication. Diethylcarbamyl chloride (90 μl, 0.70 mmol) was added and the mixture was stirred at room temperature. Further aliquots of DIPEA and diethylcarbamyl chloride were added at the time points of 30 and 60 minutes. After a total reaction time of 1.5 hour, the clear reaction mixture was applied directly onto a column of silica gel packed in 10% methanol in dichloromethane. Elution with the same solvent mixture yielded the title compound (47 mg, 79%) which was characterised as follows: MS (m/z): 424 ([M+H]⁺, 100).

### EXAMPLE 464

### Synthesis of 2-amino-4-[4-(dimethylcarbamoyl)piperazin-1-yl]-6-(4-fluorophenyl)-pyrido[3,2-d]pyrimidine

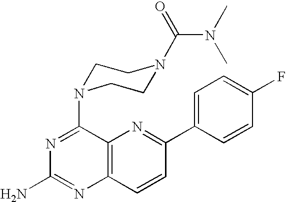

This compound was prepared according to the procedure of example 463, using dimethylcarbamyl chloride and 2-amino-4-(N-piperazin-1-yl)-6-(4-fluorophenyl)-pyrido[3,2-d]pyrimidine as starting materials. Yield: 58% for the title compound which was characterised as follows: MS (m/z): 396 ([M+H]⁺, 100).

### EXAMPLE 465

### Synthesis of 2-amino-4-[4-(diisopropylcarbamoyl)piperazin-1-yl]-6-(4-fluorophenyl)-pyrido[3,2-d]pyrimidine

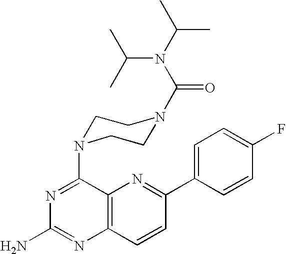

This compound was prepared according to the procedure of example 463, using diisopropylcarbamyl chloride and 2-amino-4-(N-piperazin-1-yl)-6-(4-fluorophenyl)-pyrido[3,2-d]pyrimidine as starting materials and a total reaction time of 2.5 hours. Yield: 82% for the title compound which was characterised as follows: MS (m/z): 452 ([M+H]⁺, 100).

### EXAMPLE 466

### Synthesis of 2-amino-4-[4-(morpholinocarbonyl)piperazin-1-yl]-6-(4-fluorophenyl)-pyrido[3,2-o]pyrimidine

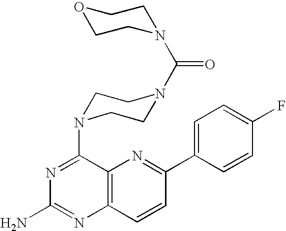

This compound was prepared according to the procedure of example 463 using 4-morpholinocarbonyl chloride and 2-amino-4-(N-piperazin-1-yl)-6-(4-fluorophenyl)-pyrido[3,2-d]pyrimidine as starting materials and a total reaction time of 45 minutes without the additional aliquots of acyl chloride and base. Yield: 69% for the title compound which was characterised as follows: MS (m/z): 438 ([M+H]⁺, 100).

### EXAMPLE 467

### Synthesis of 2-amino-4-{4-[2-(4-chlorophenyl)-3-methylbutyryl]-piperazin-1-yl}-6-(4-fluorophenyl)-pyrido[3,2-d]pyrimidine

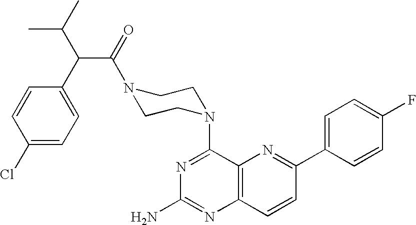

A suspension of 2-amino-4-(N-piperazin-1-yl)-6-(4-fluorophenyl)-pyrido[3,2-d]pyrimidine (68 mg, 0.21 mmol) and DIPEA (42 μl, 0.25 mmol) in dry dioxane (5 ml) was brought under an N₂ atmosphere and homogenized by brief sonication. A solution of 2-(4-chlorophenyl)-3-methylbutyryl chloride (47 μl, 0.23 mmol) in 0.5 ml of dry dioxane was added carefully. The reaction mixture was stirred for approximately 30 minutes and applied directly onto a column of silica gel packed in 10% methanol in dichloromethane. Elution with the same solvent mixture yielded the title compound (29 mg, 27%) which was characterised as follows: MS (m/z): 519, 521 ([M+H]⁺, 100).

### EXAMPLE 468

### Synthesis of 2-amino-4-[4-(2-chloropropionyl)piperazin-1-yl]-6-(4-fluorophenyl)-pyrido[3,2-d]pyrimidine

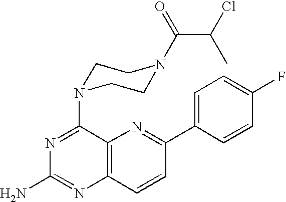

This compound was prepared according to the procedure of example 467 using 2-chloropropionyl chloride and 2-amino-4-(N-piperazin-1-yl)-6-(4-fluorophenyl)-pyrido[3,2-d]pyrimidine as starting materials. Yield: 28% for the title compound which was characterised as follows: MS (m/z): 415, 417 ([M+H]⁺, 100).

### EXAMPLE 469

### Synthesis of 2-amino-4-[4-(4-chlorophenoxycarbonyl)piperazin-1-yl]-6-(4-fluorophenyl)-pyrido[3,2-d]pyrimidine

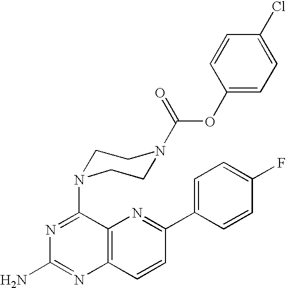

This compound was prepared according to the procedure of example 467, using 4-chlorophenyl chloroformate and 2-amino-4-(N-piperazin-1-yl)-6-(4-fluorophenyl)-pyrido[3,2-d]pyrimidine as starting materials. Yield: 72% for the title compound which was characterised as follows: MS (m/z): 479, 481 ([M+H]⁺, 100).

### EXAMPLE 470

### Synthesis of 2-amino-4-[4-(methoxycarbonylacetyl)piperazin-1-yl]-6-(4-fluorophenyl)-pyrido[3,2-d]pyrimidine

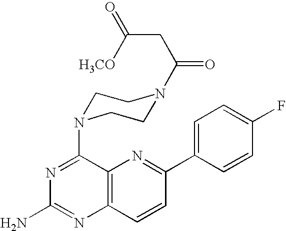

This compound was prepared according to the procedure of example 467, using methyl malonyl chloride and 2-amino-4-(N-piperazin-1-yl)-6-(4-fluorophenyl)-pyrido[3,2-d]pyrimidine as starting materials and the following work-up: the reaction mixture was partitioned between dichloromethane and water. The aqueous layer was extracted three times with dichloromethane and the combined organic layers were dried over magnesium sulfate and concentrated under reduced pressure. Purification by RP-HPLC, eluting with methanol/water 50:50+0.1% trifluoroacetic acid afforded the title compound (61 mg, 69%) which was characterised as follows: MS (m/z): 425 ([M+H]⁺, 100).

### EXAMPLE 471

### Synthesis of 2-amino-4-{4-[2-(4-chlorophenoxy)propionyl]-piperazin-1-yl}-6-(4-fluorophenyl)-pyrido[3,2-d]pyrimidine

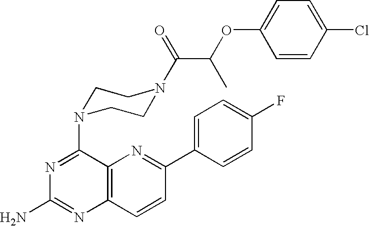

A solution of 2-amino-4-[4-(2-chloropropionyl)piperazin-1-yl]-6-(4-fluorophenyl)-pyrido[3,2-d]pyrimidine (21 mg, 51 μmol), 4-chlorophenol (10 mg, 78 μmol) and anhydrous potassium carbonate (7 mg, 51 μmol) in HPLC grade acetone (4 ml) was stirred at reflux temperature under an N₂ atmosphere for 3 days with stepwise addition of further potassium carbonate (25 mg, 0.18 mmol) and 4-chlorophenol (95 mg, 0.72 mmol). The reaction mixture was partitioned between dichloromethane and water. The aqueous layer was extracted with dichloromethane and the combined organic layers were dried over magnesium sulfate and concentrated under reduced pressure. Purification on a column of silica eluting with 10% methanol in dichloromethane afforded the title compound (12 mg, 46%) which was characterised as follows: MS (m/z): 507, 509 ([M+H]⁺, 100).

### EXAMPLE 472

### Synthesis of 2-amino-4-{4-[2-(4-chlorophenoxy)-2-methylpropionyl]-piperazin-1-yl}-6-(4-fluorophenyl)-pyrido[3,2-d]pyrimidine

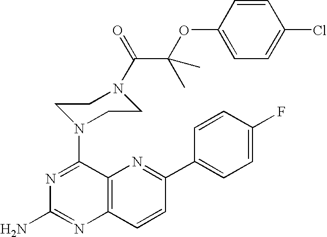

This compound was prepared according to the procedure of example 434, using 2-(4-chlorophenoxy)-2-methylpropionic acid and 2-amino-4-(N-piperazin-1-yl)-6-(4-fluorophenyl)-pyrido[3,2-d]pyrimidine as starting materials, a reaction time of 2 hours and purification on a column of silica eluting with 10% methanol in dichloromethane. Lyophilization was used to remove DMF from the chromatographically purified material. Yield: 59% for the title compound which was characterised as follows: MS (m/z): 521, 523 ([M+H]⁺, 100).

### EXAMPLE 473

### Synthesis of 2-amino-4-{4-[3-(4-chlorophenoxy)propionyl]-piperazin-1-yl}-6-(4-fluorophenyl)-pyrido[3,2-d]pyrimidine

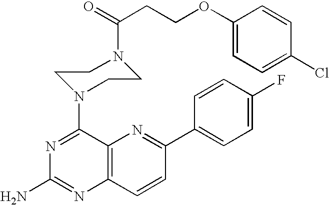

This compound was prepared according to the procedure of example 434, using 3-(4-chlorophenoxy)propionic acid and 2-amino-4-(N-piperazin-1-yl)-6-(4-fluorophenyl)-pyrido[3,2-d]pyrimidine as starting materials, dry dioxane (5 ml) as solvent, a reaction time of 1 hour and purification on a column of silica eluting with 10% methanol in dichloromethane. Yield: 64% for the title compound which was characterised as follows: MS (m/z): 507, 509 ([M+H]⁺, 100).

### EXAMPLE 474

### Synthesis of 2-amino-4-[4-(2-phenoxypropionyl)piperazin-1-yl]-6-(4-fluorophenyl)-pyrido[3,2-d]pyrimidine

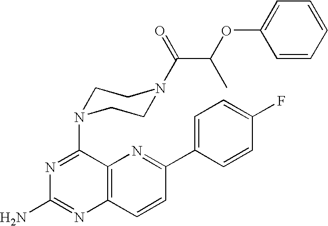

This compound was prepared according to the procedure of example 471, using phenol (77 mg, 0.82 mmol), potassium carbonate (85 mg, 0.61 mmol) and 2-amino-4-[4-(2-chloropropionyl)piperazin-1-yl]-6-(4-fluorophenyl)-pyrido[3,2-d]pyrimidine as starting materials. Yield: 70% for the title compound which was characterised as follows: MS (m/z): 473 ([M+H]⁺, 100).

### EXAMPLE 475

### Synthesis of {4-[2-amino-6-(4-fluorophenyl)-pyrido[3,2-d]pyrimidin-4-yl]-piperazin-1-yl}-acetic acid 4-chloro-benzyl ester

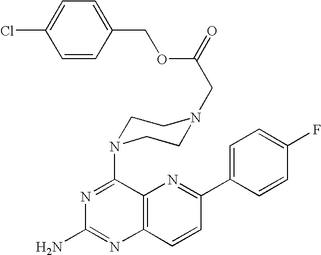

A suspension of 2-amino-4-(N-piperazin-1-yl)-6-(4-fluorophenyl)-pyrido[3,2-d]pyrimidine (45 mg, 0.14 mmol) and DIPEA (140 μl, 0.83 mmol) in dry dioxane (10 ml) was brought under an N₂ atmosphere and homogenized by brief sonication. A solution of chloroacetic acid 4-chlorobenzyl ester (100 mg, 0.46 mmol) in 1 ml of dry dioxane was added and the reaction mixture was stirred at reflux temperature under an N₂ atmosphere for 21 hours. The reaction mixture was partitioned between dichloromethane and water. The aqueous layer was extracted with dichloromethane and the combined organic layers were dried over magnesium sulfate and concentrated under reduced pressure. Purification on a column of silica eluting with 10% methanol in dichloromethane afforded the title compound (31 mg, 44%) which was characterised as follows: MS (m/z): 507, 509 ([M+H]⁺, 100).

### EXAMPLE 476

### Synthesis of N-(2-{4-[2-amino-6-(4-fluorophenyl)-pyrido[3,2-d]pyrimidin-4-yl]-piperazin-1-yl}-2-oxoethyl)-4-chlorobenzamide

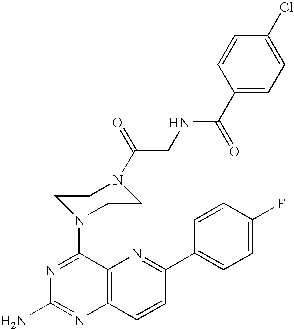

A suspension of 2-amino-4-[4-(aminoacetyl)-piperazin-1-yl]-6-(4-fluorophenyl)-pyrido[3,2-d]pyrimidine (70 mg, 0.18 mmol) and DIPEA (37 μl, 0.22 mmol) in dry dioxane (5 ml) was brought under an N₂ atmosphere and homogenized by brief sonication. 4-Chlorobenzoyl chloride (26 μl, 0.20 mmol) was added and the reaction mixture was stirred for approximately 30 minutes. Purification by chromatography on silica eluting with 7-10% methanol in dichloromethane yielded the title compound (48 mg, 51%) which was characterised as follows: MS (m/z): 520, 522 ([M+H]⁺, 100).

### EXAMPLE 477

### Synthesis of (S)-[2-{4-[2-amino-6-(4-fluorophenyl)-pyrido[3,2-d]pyrimidin-4-yl]-piperazin-1-yl}-1-(4-chlorobenzyl)-2-oxoethyl]-carbamic acid tert-butyl ester

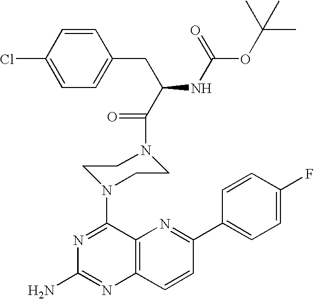

This compound was prepared according to the procedure of example 434, using N-Boc-4-chlorophenylalanine and 2-amino-4-(N-piperazin-1-yl)-6-(4-fluorophenyl)-pyrido[3,2-d]pyrimidine as starting materials, dry dioxane (5 ml) as solvent, a reaction time of 3.5 hours and the following work-up: the reaction mixture was partitioned between dichloromethane and water and the aqueous layer is extracted three times with dichloromethane. The combined organic layers are dried over magnesium sulfate, filtered and concentrated under reduced pressure. Purification by chromatography on a column of silica, eluting with 10% methanol in dichloromethane yielded the title compound (73 mg, ˜100%) which was characterised as follows: MS (m/z): 606, 608 ([M+H]⁺, 100).

### EXAMPLE 478

### Synthesis of N-(2-{4-[2-amino-6-(4-fluorophenyl)-pyrido[3,2-f]pyrimidin-4-yl]-piperazin-1-yl}-2-oxoethyl)benzamide

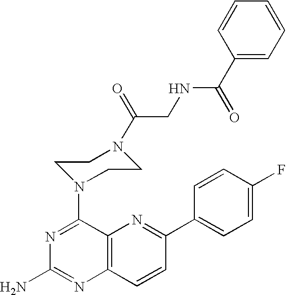

This compound was prepared according to the procedure of example 476, using benzoyl chloride and 2-amino-4-(N-piperazin-1-yl)-6-(4-fluorophenyl)-pyrido[3,2-d]pyrimidine as starting materials and purification on a column of silica eluting with 10% methanol in dichloromethane. Yield: 94% for the title compound which was characterised as follows: MS (m/z): 486 ([M+H]⁺, 100).

### EXAMPLE 479

### Synthesis of 2-amino-4-(piperidin-4-ylamino)-6-(4-fluorophenyl)-Pyrido[3,2-d]pyrimidine

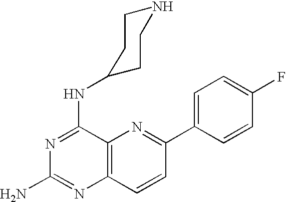

Trifluoroacetic acid (TFA; 14 ml) was added via a syringe to a solution of 2-amino-4-[1-(tert-butoxyarbonyl)piperid-4-ylamino]-6-(4-fluorophenyl)-pyrido-[3,2-d]pyrimidine (333 mg, 0.76 mmol) in dichloromethane (30 ml) cooled to 0° C. under an N₂ atmosphere. After 5 minutes, the ice bath was removed and the reaction mixture was stirred for a further 25 minutes, whereupon it was diluted with dichloromethane (30 ml) and treated with a saturated sodium hydrogen carbonate solution (250 ml). The layers were separated and the aqueous layer was extensively extracted with dichloromethane. The combined organic layers were dried over magnesium sulfate, filtered and concentrated under reduced pressure. The crude material was used in the next reaction step without further purification.

### EXAMPLE 480

### Synthesis of 2-amino-4-[1-(4-chlorophenoxyacetyl)piperid-4-ylamino]-6-(4-fluorophenyl)-pyrido[3,2-d]pyrimidine

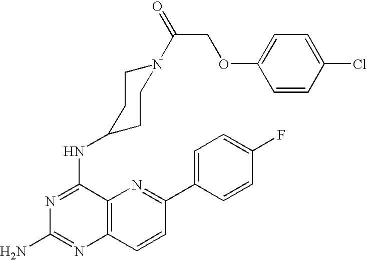

A suspension of 2-amino-4-(piperid-4-ylamino)-6-(4-fluorophenyl)-pyrido[3,2-d]pyrimidine (87 mg, 0.26 mmol) and DIPEA (52 μl, 0.31 mmol) in dry dioxane (10 ml) was brought under an N₂ atmosphere and homogenized by brief sonication. 4-Chlorophenoxyacetyl chloride (62 mg, 0.30 mmol) was added and the reaction mixture was stirred under an N₂ atmosphere for 30 minutes. The reaction mixture was partitioned between dichloromethane and water. The aqueous layer was extracted with dichloromethane and the combined organic layers were dried over magnesium sulfate and concentrated under reduced pressure. Purification on a column of silica eluting with 10% methanol in dichloromethane afforded the title compound (39 mg, 30%) which was characterised as follows: MS (m/z): 507, 509 ([M+H]⁺, 100).

### EXAMPLE 481

### Synthesis of (R)-4-[2-amino-6-(4-fluorophenyl)-pyrido[3,2-d]pyrimidin-4-yl]-3-methylpiperazine-1-carboxylic acid tert-butyl ester

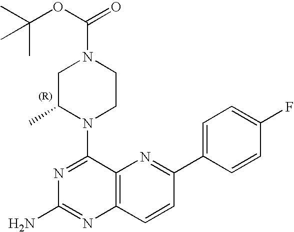

This compound was prepared according to the procedure of example 457, using (R)-1-Boc-3-methylpiperazine and 2-amino-4-oxo-6-(4-fluorophenyl)-pyrido-[3,2-d]-pyrimidine as starting materials and a reaction time of 2 hours. Yield: 22% for the title compound which was characterised as follows: MS (m/z): 439 ([M+H]⁺, 100).

### EXAMPLE 482

### Synthesis of (R)-2-amino-4-[4-(4-chlorophenoxyacetyl)-2-methylpiperazin-1-yl]-6-(4-fluorophenyl)-pyrido[3,2-d]pyrimidine

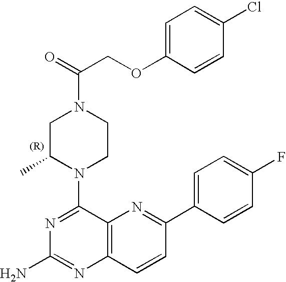

Trifluoroacetic acid (TFA; 1 ml) was added via a syringe to a solution of (R)-4-[2-amino-6-(4-fluorophenyl)-pyrido[3,2-d]pyrimidin-4-yl]-3-methylpiperazine-1-carboxylic acid tert-butyl ester (18 mg, 41 μmol) in dichloromethane (2 ml) cooled to 0° C. under an N₂ atmosphere. After 5 minutes, the ice bath was removed and the reaction mixture was stirred for a further 25 minutes, whereupon the volatiles were removed under reduced pressure. The residue was dissolved in dichloromethane (2 ml) and brought under an N₂ atmosphere. DIPEA (200 μl, 1.21 mmol) and a solution of 4-chlorophenoxyacetyl chloride (12 mg, 57 μmol) in dichloromethane (2 ml) were added and the resulting mixture was after 30 minutes applied onto a column of silica packed in 6% methanol in dichloromethane. Elution with the same solvent mixture yielded the title compound (10 mg, 48%) which was characterised as follows: MS (m/z): 507, 509 ([M+H]⁺, 100).

### EXAMPLE 483

### Synthesis of (S,S)-5-[2-amino-6-(4-fluorophenyl)-pyrido[3,2-d]pyrimidin-4-yl]-2,5-diaza-bicyclo[2.2.1]heptane-2-carboxylic acid tert-butyl ester

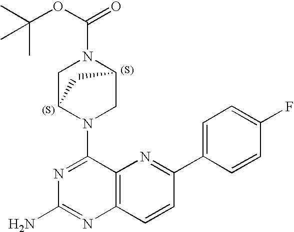

This compound was prepared according to the procedure of example 457, using (1S,4S)-2-Boc-2,5-diazabicyclo[2.2.1]heptane and 2-amino-4-oxo-6-(4-fluorophenyl)-pyrido-[3,2-d]-pyrimidine as starting materials and a reaction time of 2.5 hours. Yield: 77% for the title compound which was characterised as follows: MS (m/z): 437 ([M+H]⁺, 100).

### EXAMPLE 484

### Synthesis of (S,S)-1-[5-[2-amino-6-(4-fluorophenyl)-pyrido[3,2-d]pyrimidin-4-yl]-2,5-diaza-bicyclo[2.2.1]hept-2-yl]-2-(4-chlorophenoxy)-ethanone

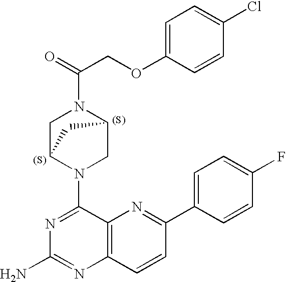

This compound was prepared according to the procedure of example 482, using (S,S)-5-[2-amino-6-(4-fluorophenyl)-pyrido[3,2-d]pyrimidin-4-yl]-2,5-diaza-bicyclo [2.2.1]heptane-2-carboxylic acid tert-butyl ester and 4-chlorophenoxyacetyl chloride as starting materials. Yield: 55% for the title compound which was characterised as follows: MS (m/z): 505, 507 ([M+H]⁺, 100).

### EXAMPLE 485

### Synthesis of 2-amino-4-[1-(phenoxyacetyl)piperid-4-ylamino]-6-(4-fluorophenyl)-pyrido[3,2-d]pyrimidine

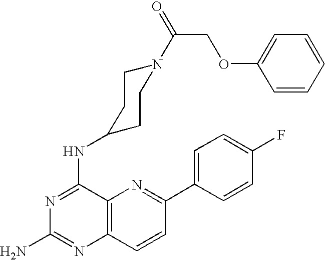

This compound was prepared according to the procedure of example 480, using 2-amino-4-(piperid-4-ylamino)-6-(4-fluorophenyl)-pyrido[3,2-pyrimidine and phenoxyacetyl chloride as starting materials and a final purification step by preparative thin layer chromatography on silica eluting with 7% methanol in dichloromethane. Yield: 19% for the title compound which was characterised as follows: MS (m/z): 473 ([M+H]⁺, 100).

### EXAMPLE 486

### Synthesis of (S)-4-[2-amino-6-(4-fluorophenyl)-pyrido[3,2-d]pyrimidin-4-yl]-3-methylpiperazine-1-carboxylic acid benzyl ester

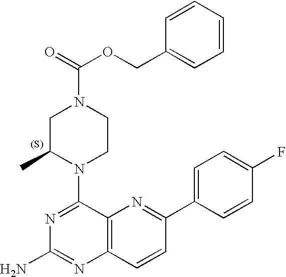

This compound was prepared according to the procedure of example 457, using 1-benzyloxycarbonyl-(S)-3-methylpiperazine and 2-amino-4-oxo-6-(4-fluorophenyl)-pyrido-[3,2-d]-pyrimidine as starting materials and a reaction time of 4 hours. The extraction step was omitted; instead, solvent was removed under reduced pressure and the residue was taken in dichloromethane and applied onto a plate of silica. Eluting with 10% methanol in dichloromethane yielded the title compound (19 mg, 21%) which was characterised as follows: MS (m/z): 473 ([M+H]⁺, 100).

### EXAMPLE 487

### Synthesis of (R)-2-amino-4-(4-benzoyl-2-methylpiperazin-1-yl)-6-(4-fluorophenyl)-pyrido[3,2-d]pyrimidine

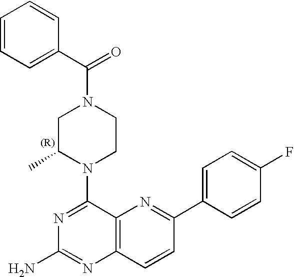

This compound was prepared according to the procedure of example 457, using (R)-1-benzoyl-3-methylpiperazine hydrochloride, 2-amino-4-oxo-6-(4-fluoro-phenyl)-pyrido-[3,2-d]-pyrimidine and DBU (138 μl, 0.90 mmol) as starting materials, a reaction time of 21 hours and a final purification step by preparative thin layer chromatography on silica eluting with 7% methanol in dichloromethane. Yield: 46% for the title compound which was characterised as follows: MS (m/z): 443 ([M+H]⁺, 100).

### EXAMPLE 488

### Synthesis of (S)-2-amino-4-[4-[3-(4-chlorophenyl)-2-aminopropionyl]piperazin-1-yl]-6-(4-fluorophenyl)-pyrido[3,2-d]pyrimidine

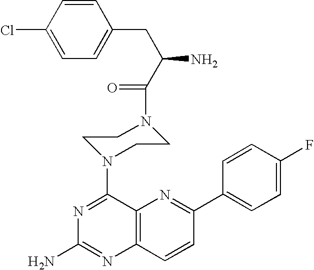

This compound was prepared according to the TFA treatment as described for the procedure of example 482, using (S)-[2-{4-[2-amino-6-(4-fluorophenyl)-pyrido[3,2-d]pyrimidin-4-yl]-piperazin-1-yl}-1-(4-chlorobenzyl)-2-oxoethyl]-carbamic acid tert-butyl ester as starting material and a total reaction time of 1 hour. The crude product was purified by preparative thin layer chromatography on silica eluting with 5% methanol 1% triethylamine in dichloromethane. Yield: 36% for the title compound which was characterised as follows: MS (m/z): 506, 508 ([M+H]⁺, 100).

### EXAMPLE 489

### Synthesis of (S)-2-amino-4-[4-(4-chlorophenoxyacetyl)-2-methylpiperazin-1-yl]-6-(4-fluorophenyl)-pyrido[3,2-d]pyrimidine

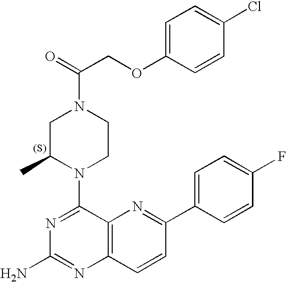

This compound was prepared according to the procedure of example 482, using (S)-4-[2-amino-6-(4-fluorophenyl)-pyrido[3,2-d]pyrimidin-4-yl]-3-methylpiperazine-1-carboxylic acid tert-butyl ester and 4-chlorophenoxyacetyl chloride as starting materials and a final purification step by preparative thin layer chromatography on silica eluting with 7% methanol in dichloromethane. Yield 61% for the title compound which was characterised as follows: MS (m/z): 507, 509 ([M+H]⁺, 100).

### EXAMPLE 490

### Synthesis of 2-amino-4-[4-(4-chlorophenylcarbamoyl-acetyl)piperazin-1-yl]-6-(4-fluorophenyl)-pyrido[3,2-d]pyrimidine

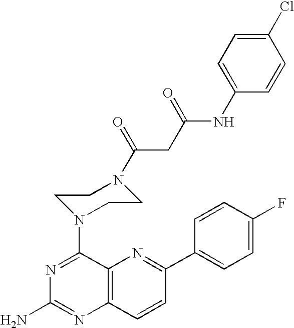

A suspension of 2-amino-4-[4-(methoxycarbonylacetyl)piperazin-1-yl]-6-(4-fluorophenyl)-pyrido[3,2-d]pyrimidine (128 mg, 0.30 mmol), 4-chloroaniline (200 mg, 1.54 mmol) and DIPEA (55 μl, 033 mmol) in 15 ml of dry dioxane was stirred at reflux temperature under an N₂ atmosphere for 5 days. The reaction mixture was partitioned between dichloromethane (50 ml) and water (50 ml). The aqueous layer was extracted twice with dichloromethane and the combined organic layers were dried over magnesium sulfate, filtered and concentrated under reduced pressure. Chromatography on silica eluting with mixtures of dichloromethane and methanol (5-10% methanol) afforded the crude title product (46 mg, 29%) for the title compound which was characterised as follows: MS (m/z): 520, 522 ([M+H]⁺, 100).

### EXAMPLE 491

### Synthesis of 2-amino-4-piperazino-6-chloro-pyrido[3,2-d]pyrimidine

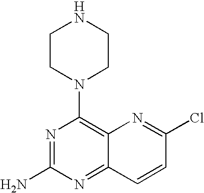

To a solution of 2-amino-6-chloro-pyrido[3,2-d]pyrimidin-4(3H)-one (example 20; 0.9 mmol, 1 g) and 1,8-diazabicyclo[5.4.0]undec-7-ene (DBU, 9.16 mmol, 1.368 mL) in DMF (80 mL) were added (benzotriazol-1-yloxy)tris-(dimethylamino)phosphonium hexafluorophosphate (BOP, 7.63 mmol, 3.375 g) and piperazine (22.89 mmol, 1.972 g). The reaction mixture was stirred at room temperature for 16 hours after which the solvent was evaporated in vacuo. The residue was adsorbed on silica and purified by silica gel column chromatography (the mobile phase being a dichloromethane/methanol/triethylamine mixture, in a ratio gradually ranging from 98:1:1 to 95:4:1) yielding the title compound (734 mg, yield 54%), which was characterized by its mass spectrum as follows: MS (m/z): 265 ([M+H]⁺, 100).

### EXAMPLE 492

### Preparation of 2-amino-4-(N-(4-chlorophenoxyacetyl)-piperazin-1-yl)-6-chloro-pyrido[3,2-d]pyrimidine

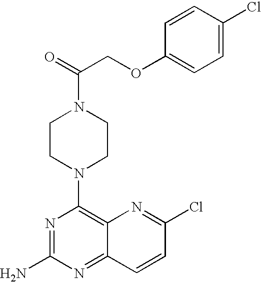

To a solution of 2-amino-4-piperazino-6-chloro-pyrido[3,2-d]pyrimidine (2.77 mmol, 734 mg) and N,N-diisopropylethylamine (6.10 mmol, 1 mL) in dioxane (120 mL) and methanol (30 mL) was added a solution of 4-chlorophenoxyacetyl chloride (3.05 mmol, 625 mg) in-dioxane (30 mL). The reaction mixture was stirred at room temperature for 16 hours. Then, the solvents were evaporated in vacuo and the residue was purified by means of silica gel column chromatography (the mobile phase being a dichloromethane/methanol mixture, in a ratio gradually ranging from 99:1 to 97:3) yielding the pure title compound (906 mg, yield 75%), which was characterized by its mass spectrum as follows: MS (m/z): 433 ([M+H]⁺, 100).

### EXAMPLES 493 TO 515

### Synthesis of 2-amino-4-[N-(4-chlorophenoxyacetyl)-piperazin-1-yl]-6-aryl-pyrido(3,2-d)pyrimidine analogues

To a solution of 2-amino-4-(N-(4-chlorophenoxyacetyl)-piperazin-1-yl)-6-chloro-pyrido[3,2-d]pyrimidine (0.23 mmol, 100 mg) and potassium fluoride (0.58 mmol, 33 mg) in dioxane (8 mL) and water (3 mL) was added the appropriate arylboronic or heteroarylboronic acid, or ester thereof (0.25 mmol) and tetrakis(triphenyl-phosphine)palladium(0) (0.01 mmol, 13 mg). The reaction mixture was heated at 90° C. for 4 to 7 hours (following the course of the reaction by thin layer chromatography), cooled down to room temperature and extracted with dichloromethane. The organic layer was evaporated in vacuo and the residue was purified by silica gel column chromatography (the mobile phase being a dichloromethane/methanol mixture, in a ratio gradually ranging from 99:1 to 97:3) yielding the pure, corresponding compounds (yields ranging from 45 to 91%).

The following compounds were synthesized and characterised according to this procedure:

### EXAMPLE 493

### 2-amino-4-[N-(4-chlorophenoxyacetyl)-piperazin-1-yl]-6-(3-chloro-4-ethoxyphenyl)-pyrido(3,2-d)pyrimidine

This compound was obtained from 3-chloro-4-ethoxyphenylboronic acid in 62% yield and was characterized by its mass spectrum as follows: MS (m/z): 553 ([M+H]⁺, 100).

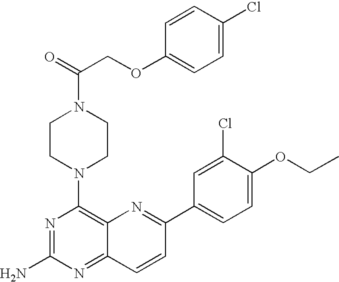

### EXAMPLE 494

### 2-amino-4-[N-(4-chlorophenoxyacetyl)-piperazin-1-yl]-6-(4-ethoxy phenyl)-pyrido(3,2-d)pyrimidine

This compound was obtained from 4-ethoxyphenylboronic acid in 69% yield and was characterized by its mass spectrum as follows: MS (m/z): 519 ([M+H]⁺, 100).

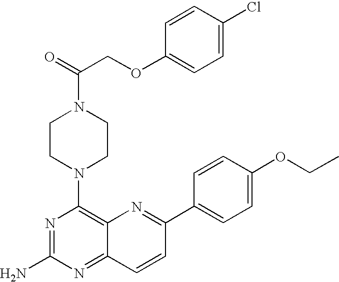

### EXAMPLE 495

### 2-amino-4-[N-(4-chlorophenoxyacetyl)-piperazin-1-yl]-6-(4-methyl thiophenyl)-pyrido(3,2-d)pyrimidine

This compound was obtained from 4-methylthiophenylboronic acid in 61% yield and was characterized by its mass spectrum as follows: MS (m/z): 521 ([M+H]⁺, 100).

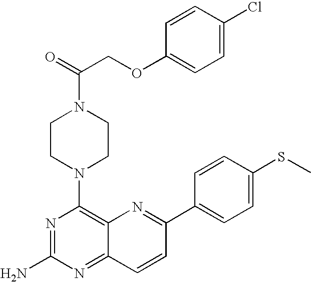

### EXAMPLE 496

### 2-amino-4-[N-(4-chlorophenoxyacetyl)-piperazin-1-yl]-6-(3-methoxy-4-hydroxyphenyl)-pyrido(3,2-d)pyrimidine

This compound was obtained from 2-methoxy-4-(4,4,5,5-tetramethyl-1,3,2-dioxaborolan-2-yl)phenol in 58% yield and was characterized by its mass spectrum as follows: MS (m/z): 521 ([M+H]⁺, 100).

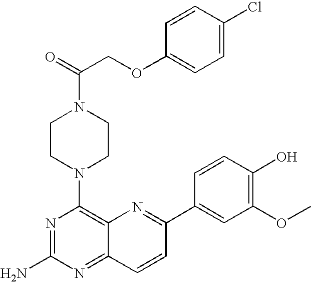

### EXAMPLE 497

### 2-amino-4-[N-(4-chlorophenoxyacetyl)-piperazin-1-yl]-6-(2,3-dihydro-1-benzofuran-5-yl)-pyrido(3,2-d)pyrimidine

This compound was obtained from 2,3-dihydro-1-benzofuran-5-ylboronic acid in 52% yield and was characterized by its mass spectrum as follows: MS (m/z): 517 ([M+H]⁺, 100).

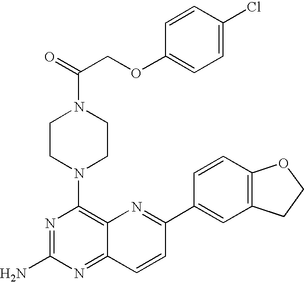

### EXAMPLE 498

### 2-amino-4-[N-(4-chlorophenoxyacetyl)-piperazin-1-yl]-6-(3-methyl-phenyl)-pyrido(3,2-d)pyrimidine

This compound was obtained from 3-methylphenylboronic acid in 69% yield and was characterized by its mass spectrum as follows: MS (m/z): 489 ([M+H]⁺, 100).

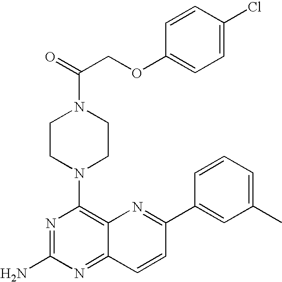

### EXAMPLE 499

### 2-amino-4-[N-(4-chlorophenoxyacetyl)-piperazin-1-yl]-6-(4-cyanomethyl-phenyl)-pyrido(3,2-d)pyrimidine

This compound was obtained from 2-[(4-cyanomethyl)phenyl]-4,4,5,5-tetramethyl-1,3,2-dioxaborolane in 62% yield and was characterized by its mass spectrum as follows: MS (m/z): 514 ([M+H]⁺, 100).

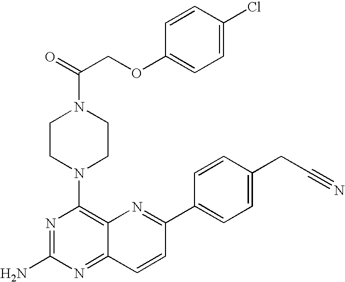

### EXAMPLE 500

### 2-amino-4-[N-(4-chlorophenoxyacetyl)-piperazin-1-yl]-6-(3-methoxy-methylphenyl)-pyrido(3,2-d)pyrimidine

This compound was obtained from 2-[(3-methoxymethyl)phenyl]-4,4,5,5-tetramethyl-1,3,2-dioxaborolane in 67% yield and was characterized by its mass spectrum as follows: MS (m/z): 519 ([M+H]⁺, 100).

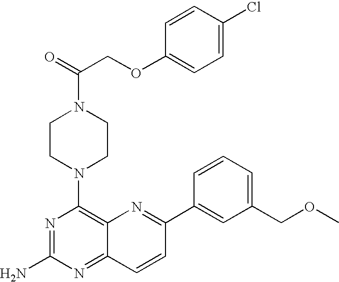

### EXAMPLE 501

### 2-amino-4-[N-(4-chlorophenoxyacetyl)-piperazin-1-yl]-6-(3-methoxy-4-benzyloxyphenyl)-pyrido(3,2-d)pyrimidine

This compound was obtained from 2-[(3-methoxy-4-benzyloxymethyl)phenyl]-4,4,5,5-tetramethyl-1,3,2-dioxaborolane in 63% yield (0.25 mmol, 86 mg) and was characterized by its mass spectrum as follows: MS (m/z): 611 ([M+H]⁺, 100).

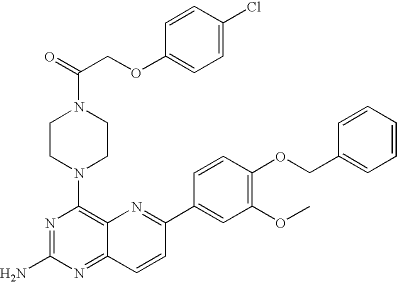

### EXAMPLE 502

### 2-amino-4-[N-(4-chlorophenoxyacetyl)-piperazin-1-yl]-6-(3,5-dimethyl-4-methoxyphenyl)-pyrido(3,2-d)pyrimidine

This compound was obtained from 3,5-dimethyl-4-methoxyphenylboronic acid in 73% yield and was characterized by its mass spectrum as follows: MS (m/z): 533 ([M+H]⁺, 100).

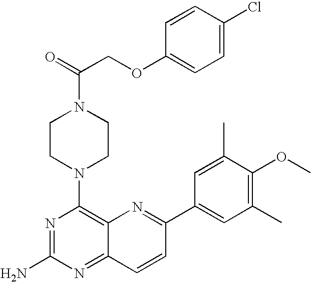

### EXAMPLE 503

### 2-amino-4-[N-(4-chlorophenoxyacetyl)-piperazin-1-yl]-6-(4-cyano-methoxyphenyl)-pyrido(3,2-d)pyrimidine

This compound was obtained from 2-[(4-cyanomethoxy)phenyl]-4,4,5,5-tetramethyl-1,3,2-dioxaborolane in 76% yield and was characterized by its mass spectrum as follows: MS (m/z): 530 ([M+H]⁺, 100).

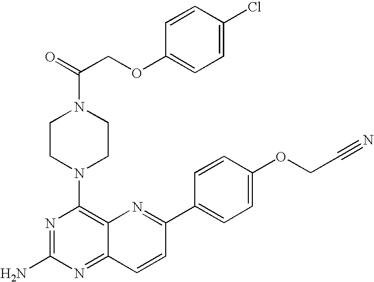

### EXAMPLE 504

### 2-amino-4-[N-(4-chlorophenoxyacetyl)-piperazin-1-yl]-6-(4-acetoxy-Phenyl)-pyrido(3,2-d)pyrimidine

This compound was obtained from 4-(4,4,5,5-tetramethyl-1,3,2-dioxaborolan-2-yl)phenyl acetate in 45% yield and was characterized by its mass spectrum as follows:

MS (m/z): 533 ([M+H]⁺, 100).

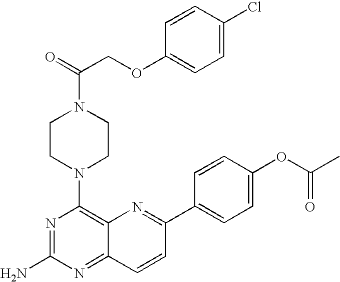

### EXAMPLE 505

### 2-amino-4-[N-(4-chlorophenoxyacetyl)-piperazin-1-yl-6-(2,4-dimethoxyphenyl)-pyrido(3,2-d)pyrimidine

This compound was obtained from 2,4-dimethoxyphenylboronic acid in 89% yield and was characterized by its mass spectrum as follows: MS (m/z): 535 ([M+H]⁺, 100).

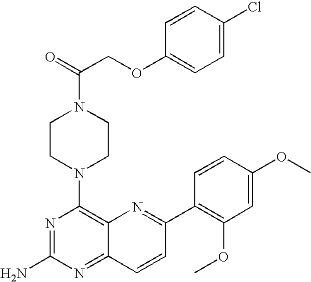

### EXAMPLE 506

### 2-amino-4-[N-(4-chlorophenoxyacetyl)-piperazin-1-yl]-6-(2,5-dimethoxyphenyl)-pyrido(3,2-d)pyrimidine

This compound was obtained from 2,5-dimethoxyphenylboronic acid in 91% yield and was characterized by its mass spectrum as follows: MS (m/z): 535 ([M+H]⁺, 100).

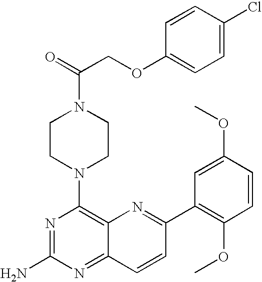

### EXAMPLE 507

### 2-amino-4-[N-(4-chlorophenoxyacetyl)-piperazin-1-yl]-6-(3-amino-4-methylphenyl)-pyrido(3,2-d)pyrimidine

This compound was obtained from 3-amino-4-methylphenylboronic acid in 84% yield and was characterized by its mass spectrum as follows: MS (m/z): 504 ([M+H]⁺, 100).

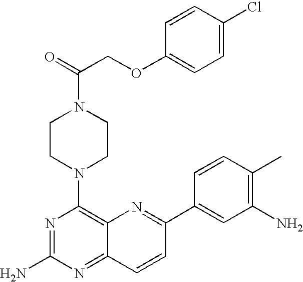

### EXAMPLE 508

### Synthesis of 2-amino-4-[N-(4-chlorophenoxyacetyl)-piperazin-1-yl]-6-(4-ethylphenyl)-pyrido(3,2-d)pyrimidine

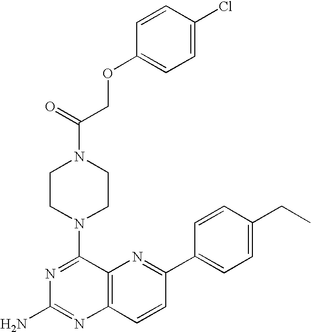

This compound was obtained from 4-ethylphenylboronic acid in 72% yield and was characterized by its mass spectrum as follows: MS (m/z): 503 ([M+H]⁺, 100).

### EXAMPLE 509

### Synthesis of 2-amino-4-[N-(4-chlorophenoxyacetyl)-piperazin-1-yl]-6-(3-methyl-4-methoxyphenyl)-pyrido(3,2-d)pyrimidine

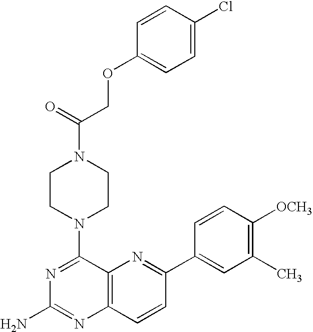

This compound was obtained from 4-methoxy-3-methylphenylboronic acid in 68% yield and was characterized by its mass spectrum as follows: MS (m/z): 519 ([M+H]⁺, 100).

### EXAMPLE 510

### Synthesis of 2-amino-4-[N-(4-chlorophenoxyacetyl)-piperazin-1-yl]-6-(4-ethoxycarbonylphenyl)-pyrido(3,2-d)pyrimidine

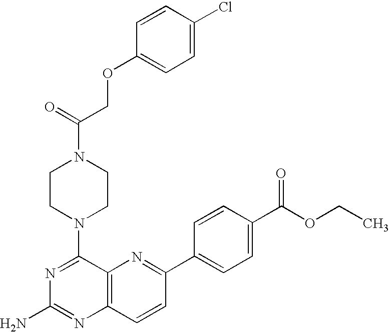

This compound was obtained from in (4-ethoxycarbonylphenyl)boronic acid in 77% yield and was characterized by its mass spectrum as follows: MS (m/z): 547 ([M+H]⁺, 100).

### EXAMPLE 511

### Synthesis of 2-amino-4-[N-(4-chlorophenoxyacetyl)-piperazin-1-yl]-6-(4-methyl-2-thienyl)-pyrido[3,2-d]pyrimidine

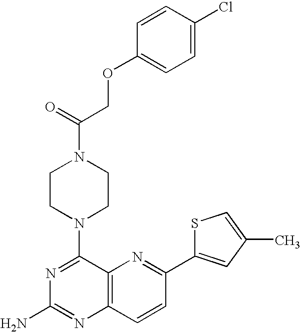

This compound was obtained from 4-methylthiophene-2-boronic acid in 59% yield and was characterized by its mass spectrum as follows: MS (m/z): 495 ([M+H]⁺, 100).

### EXAMPLE 512

### Synthesis of 2-amino-4-[N-(4-chlorophenoxyacetyl)-piperazin-1-yl]-6-(2-methyl-phenyl)-pyrido(3,2-d)pyrimidine

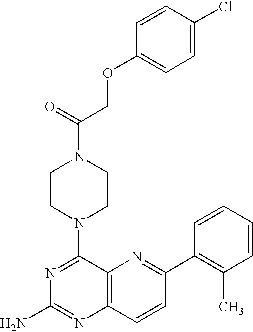

This compound was obtained from 2-methylphenylboronic acid in 81% yield and was characterized by its mass spectrum as follows: MS (m/z): 489 ([M+H]⁺, 100).

### EXAMPLE 513

### Synthesis of 2-amino-4-[N-(4-chlorophenoxyacetyl)-piperazin-1-yl]-6-(3-fluoro-4-benzyloxy-phenyl)-pyrido(3,2-d)pyrimidine

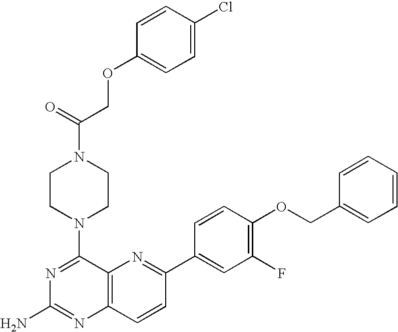

This compound was obtained from 4-benzyloxy-3-fluorophenylboronic acid in 86% yield and was characterized by its mass spectrum as follows: MS (m/z): 599 ([M+H]⁺, 100).

### EXAMPLE 514

### Synthesis of 2-amino-4-[N-(4-chlorophenoxyacetyl)-piperazin-1-yl]-6-(3-methoxy-4-amino-phenyl)-pyrido[3,2-d]pyrimidine

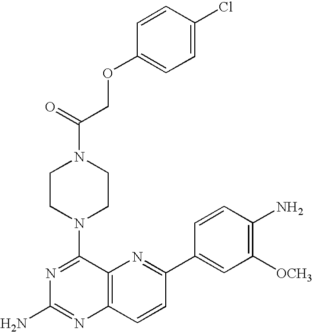

This compound was obtained from 4-amino-3-methoxyphenylboronic acid in 54% yield and was characterized by its mass spectrum as follows: MS (m/z): 520 ([M+H]⁺, 100).

### EXAMPLE 515

### Synthesis of 2-amino-4-[N-(4-chlorophenoxyacetyl)-piperazin-1-yl]-6-(3-methoxy-4-acetoxy-phenyl)-pyrido(3,2-d)pyrimidine

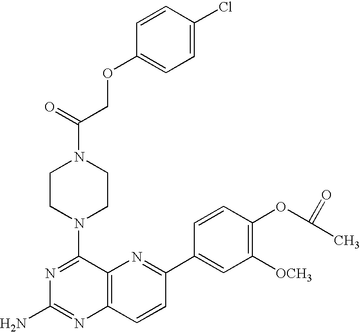

This compound was obtained from 4-acetoxycarbonyl-3-methoxyphenyl boronic acid pinacol ester in 69% yield and was characterized by its mass spectrum as follows: MS (m/z): 563 ([M+H]⁺, 100).

### EXAMPLES 516 TO 522

### Synthesis of 2-amino-4-[N-(4-chlorophenoxyacetyl)-piperazin-1-yl]-6-aryl-pyrido(3,2-d)pyrimidine analogues

To a solution of 2-amino-4-(N-(4-chlorophenoxyacetyl)-piperazin-1-yl)-6-chloro-pyrido[3,2-d]pyrimidine (0.2 mmol, 86 mg) in dioxane (6 ml) was added the appropriate arylboronic acid (0.22 mmol). A solution of potassium carbonate (170 mg) in water (2 ml) was added to the first solution. Tetrakis(triphenylphosphine)palladium(0) (40 mg) was added and the reaction mixture was heated at 75° C. overnight. The solvents were evaporated in vacuo and the residue was purified by preparative thin layer chromatography, the mobile phase being a mixture of dichloromethane/methanol mixture (in a ratio from 90:10) yielding the pure title compounds (yields ranging from 45 to 75%) which were characterised as follows.

### EXAMPLE 516

### Synthesis of 2-amino-4-[N-(4-chlorophenoxyacetyl)-piperazin-1-yl]-6-(3,4-dimethyl-phenyl)-pyrido(3,2-d)pyrimidine

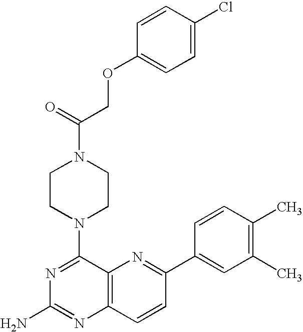

This compound was obtained from 3,4-dimethylphenyl boronic acid in 69% yield and was characterized by its mass spectrum as follows: MS (m/z): 503 ([M+H]⁺, 100.

### EXAMPLE 517

### Synthesis of 2-amino-4-[N-(4-chlorophenoxyacetyl)-piperazin-1-yl]-6-(3,4-dimethyl-phenyl)-pyrido(3,2-d)pyrimidine

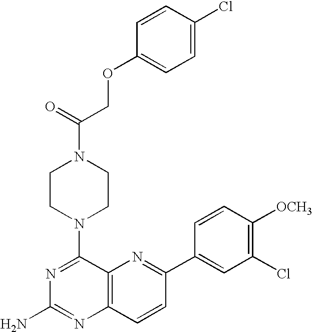

This compound was obtained from 3-chloro-4-methoxyphenyl boronic acid in 71% yield and was characterized by its mass spectrum as follows: MS (m/z): 539 ([M+H]⁺, 100.

### EXAMPLE 518

### Synthesis of 2-amino-4-[N-(4-chlorophenoxyacetyl)-piperazin-1-yl]-6-(3-ethoxy-4-fluoro-phenyl)-pyrido(3,2-d)pyrimidine

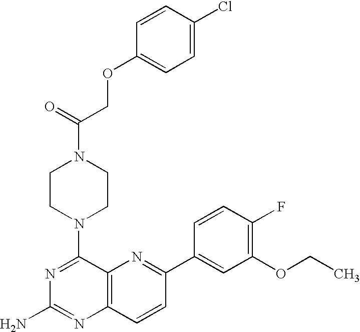

This compound was obtained from 4-fluoro-3-ethoxyphenyl boronic acid in 62% yield and was characterized by its mass spectrum as follows: MS (m/z): 537 ([M+H]⁺, 100).

### EXAMPLE 519

### Synthesis of 2-amino-4-[N-(4-chlorophenoxyacetyl)-piperazin-1-yl]-6-(3-methoxy-4-acetoxy-phenyl)-pyrido(3,2-d)pyrimidine

This compound was obtained from 4-methoxycarbonyl-3-methoxyphenyl boronic acid pinacol ester in 78% yield and was characterized by its mass spectrum as follows: MS (m/z): 563 ([M+H]⁺, 100).

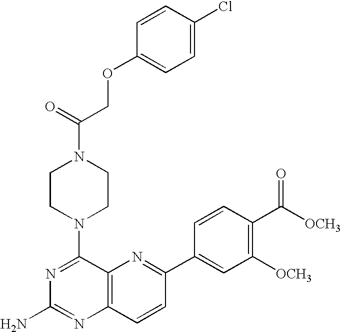

### EXAMPLE 520

### Synthesis of 2-amino-4-[N-(4-chlorophenoxyacetyl)-piperazin-1-yl]-6-(4-isobutyl-phenyl)-pyrido(3,2-d)pyrimidine

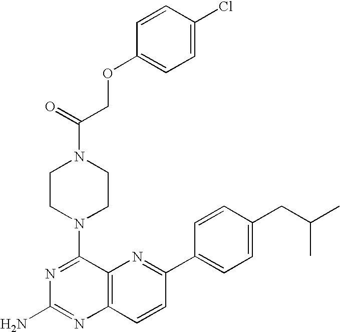

This compound was obtained from 4-isobutylphenyl boronic acid in 83% yield and was characterized by its mass spectrum as follows: MS (m/z): 531 ([M+H]⁺, 100).

### EXAMPLE 521

### Synthesis of 2-amino-4-[N-(4-chlorophenoxyacetyl)-piperazin-1-yl]-6-(3-hydroxy-4-methoxy-phenyl)-pyrido(3,2-d)pyrimidine

This compound was obtained from 4-methoxy-3-hydroxyphenyl boronic acid pinacol ester in 59% yield and was characterized by its mass spectrum as follows: MS (m/z): 521 ([M+H]⁺, 100).

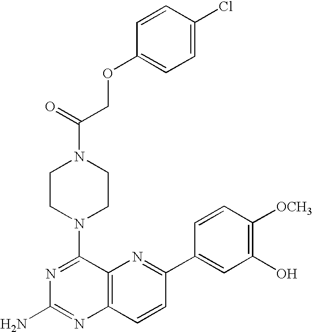

### EXAMPLE 522

### Synthesis of 2-amino-4-[N-(4-chlorophenoxyacetyl)-piperazin-1-yl]-6-(4-isopropoxy-phenyl)-pyrido(3,2-d)pyrimidine

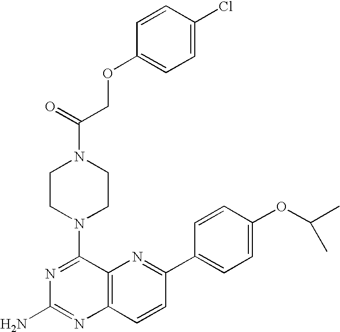

This compound was obtained from 4-isopropoxyphenyl boronic acid in 77% yield and was characterized by its mass spectrum as follows: MS (m/z): 533 ([M+H]⁺, 100).

### EXAMPLE 523

### Synthesis of [4-(2-amino-6-chloropyrido[3,2-d]pyrimidin-4-yl)piperazin-1-yl]chroman-2-ylmethanone

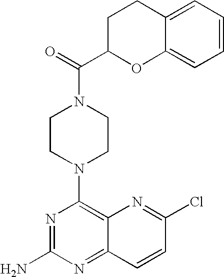

To a solution of chromane-2-carboxylic acid (0.91 mmol, 161 mg) and N,N-diisopropylethylamine (0.91 mmol, 150 μL) in dioxane (25 mL) was added O-(benzotriazol-1-yl)-N,N,N′,N′-tetramethyluronium tetrafluoroborate (TBTU, 0.91 mmol, 291 mg). After 5 minutes of stirring at room temperature, 2-amino-4-piperazino-6-chloro-pyrido[3,2-d]pyrimidine (0.75 mmol, 200 mg) was added whereupon the mixture was sonicated for 1 minute. The reaction mixture was stirred for 16 hours at room temperature, the solvent was evaporated in vacuo and the crude residue was purified by means of silica gel column chromatography (the mobile phase being a dichloromethane/methanol mixture, in a ratio gradually ranging from 99:1 to 97:3) yielding the pure title compound (234 mg, yield 73%), which was characterized by its mass spectrum as follows: MS (m/z): 425 ([M+H]⁺, 100).

### EXAMPLES 524 TO 525

### Synthesis of [4-(2-amino-6-arylpyrido[3,2-d]pyrimidin-4-yl)piperazin-1-yl]chroman-2-ylmethanone

To a solution of [4-(2-amino-6-chloropyrido[3,2-d]pyrimidin-4-yl)piperazin-1-yl]chroman-2-ylmethanone (0.12 mmol, 50 mg) and potassium fluoride (0.29 mmol, 17 mg) in dioxane (4 mL) and water (1.5 mL) was added the appropriate arylboronic acid (0.13 mmol) and tetrakis(triphenylphosphine)palladium(0) (0.006 mmol, 7 mg). The reaction mixture was heated at 90° C. for 24 hours, cooled down to room temperature and extracted with dichloromethane. The organic layer was evaporated in vacuo and the residue was purified by silica gel column chromatography (the mobile phase being a dichloromethane/methanol mixture, in a ratio gradually ranging from 99:1 to 97:3) yielding the pure, corresponding compounds (yield 53%) which were characterised as follows.

### EXAMPLE 524

### Synthesis of [4-(2-amino-6-(4-fluorophenyl)pyrido[3,2-d]pyrimidin-4-yl)piperazin-1-yl]chroman-2-ylmethanone

This compound was obtained from 4-fluorophenylboronic acid in 53% yield and was characterized by its mass spectrum as follows: MS (m/z): 485 ([M+H]⁺, 100).

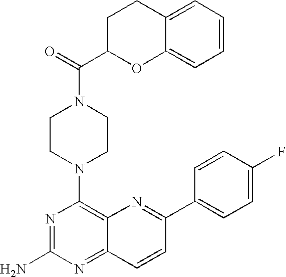

### EXAMPLE 525

### Synthesis of [4-(2-amino-6-p-tolylpyrido[3,2-d]pyrimidin-4-yl)piperazin-1-yl]chroman-2-ylmethanone

This compound was obtained from p-tolylboronic acid in 53% yield and was characterized by its mass spectrum as follows: MS (m/z): 481 ([M+H]⁺, 100).

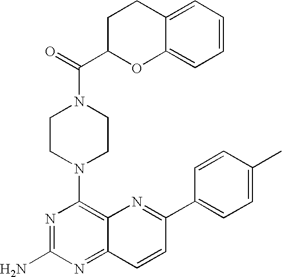

### EXAMPLE 526

### Synthesis of 2-carboxamide-3-amino-6-(4-fluorophenyl)pyridine

To a solution of 2-carboxamide-3-amino-6-chloropyridine (11.6 mmol, 2.0 g) and potassium carbonate (29.1. mmol, 4.03 g) in dioxane (240 mL) and water (90 mL) was added 4-fluorophenylboronic acid (12.8 mmol, 1.79 g) and tetrakis (triphenylphosphine)palladium(0) (0.58 mmol, 673 mg). The reaction mixture was heated at 90° C. for 6 hours, cooled down to room temperature and extracted with dichloromethane. The organic layer was evaporated in vacuo and the residue was purified by silica gel column chromatography (the mobile phase being a dichloromethane/methanol mixture of 99.75:0.25) yielding the pure title compound (2.237 g, yield 83%) which was characterized by its mass spectrum as follows: MS (m/z): 232 ([M+H]⁺, 100).

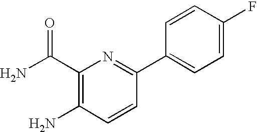

### EXAMPLES 527 TO 529

### Synthesis of 2-substituted-6-(4-fluorophenyl)-pyrido[3,2-d]pyrimidin-4(3H)-one analogues

A suspension of 2-carboxamide-3-amino-6-(4-fluorophenyl)pyridine (1.3 mmol, 300 mg) and the appropriate ortho ester (5 mL) was heated at 140° C. for 24 hours. After cooling down the reaction mixture, the solids formed were filtered off, rinsed with diethyl ether and dried to the air (yields ranging from 58% to 89%). The following compounds were synthesized according to this procedure and were used as such without further purification:

### EXAMPLE 527

### 2-methyl-6-(4-fluorophenyl)-pyrido[3,2-d]pyrimidin-4(3H)-one

This compound was obtained from ethyl ortho-acetate in 58% yield and was characterized by its mass spectrum as follows: MS (m/z): 256 ([M+H]⁺, 100).

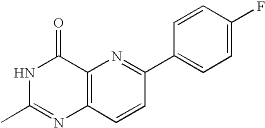

### EXAMPLE 528

### 2-ethyl-6-(4-fluorophenyl)-pyrido[3,2-d]pyrimidin-4(3H)-one

This compound was obtained from ethyl ortho-propionate in 63% yield and was characterized by its mass spectrum as follows: MS (m/z): 270 ([M+H]⁺, 100).

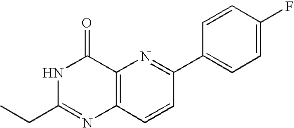

### EXAMPLE 529

### 2-phenyl-6-(4-fluorophenyl)-pyrido[3,2-d]pyrimidin-4(3H)-one

This compound was obtained from ethyl ortho-benzoate in 89% yield and was characterized by its mass spectrum as follows: MS (m/z): 318 ([M+H]⁺, 100).

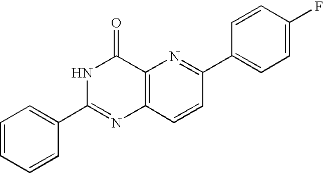

### EXAMPLE 530

### Synthesis of 2-chloromethyl-6-(4-fluorophenyl)-pyrido[3,2-d]pyrimidin-4(3H)-one

To a suspension of 2-carboxamide-3-amino-6-(4-fluorophenyl)pyridine (4.3 mmol, 1.0 g) in o-xylene (10 mL) was added 2-chloro-1,1,1-triethoxyethane (4.7 mmol, 935 mg). The reaction mixture was heated at 145° C. for 4 hours, cooled down and the solids formed were filtered off, rinsed with diethyl ether and dried to the air (1.035 g, yield 87%). The resulting title compound was characterized by its mass spectrum as follows: MS (m/z): 290 ([M+H]⁺, 100).

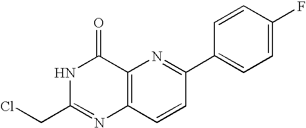

### EXAMPLES 531-533

### Synthesis of 2-substituted-4-[N-(3-methylphenyl-carbamoyl)piperazin-1-yl]-6-(4-fluorophenyl)-pyrido[3,2-d]pyrimidine analogues

To a solution of 2-substituted-6-(4-fluorophenyl)-pyrido[3,2-d]pyrimidin-4(3H)-one (1.0 mmol) and N,N-diisopropylethylamine (2.2 mmol, 377 μL) in toluene (10 mL) was added POCl₃ (1.5 mmol, 137 μL). The reaction mixture was stirred at 110° C. for 5 hours, cooled down to room temperature and extracted with dichloromethane and 2N hydrogen chloride. The organic phase was dried over magnesium sulfate and reduced in vacuo. The residue was dissolved in dichloromethane (25 mL) and piperazine (4.0 mmol, 86 mg), followed by N,N-diisopropylethylamine (2.0 mmol, 342 μL) were added. The reaction mixture was stirred at room temperature for 16 hours whereupon the solvent was evaporated in vacuo. The residue was dissolved in dichloromethane (25 mL) and m-tolyl isocyanate (2.0 mmol, 258 μL) was added. The mixture was stirred at room temperature for 6 hours. Then, the solvent was evaporated in vacuo and the crude reaction mixture was purified by means of silica gel column chromatography (the mobile phase being a dichloromethane/methanol mixture, in a ratio gradually ranging from 99:1 to 97:3) yielding the corresponding pure title compounds (yields ranging from 54% to 91%) which were characterised as follows.

### EXAMPLE 531

### 2-methyl-4-[N-(3-methylphenylcarbamoyl)piperazin-1-yl]-6-(4-fluoro-phenyl)-pyrido[3,2-d]pyrimidine

This compound was obtained in 85% yield and was characterized by its mass spectrum as follows: MS (m/z): 457 ([M+H]⁺, 100).

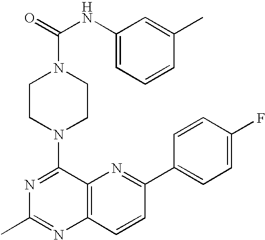

### EXAMPLE 532

### 2-ethyl-4-[N-(3-methylphenylcarbamoyl)piperazin-1-yl-6-(4-fluoro-phenyl)-pyrido[3,2-d]pyrimidine

This compound was obtained in 54% yield and was characterized by its mass spectrum as follows: MS (m/z): 471 ([M+H]⁺, 100).

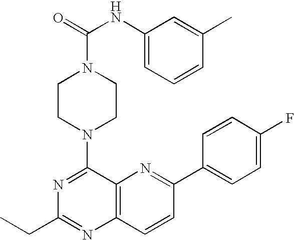

### EXAMPLE 533

### 2-phenyl-4-[N-(3-methylphenylcarbamoyl)piperazin-1-yl]-6-(4-fluoro-phenyl)-pyrido[3,2-d]pyrimidine

This compound was obtained in 91% yield and was characterized by its mass spectrum as follows: MS (m/z): 519 ([M+H]⁺, 100).

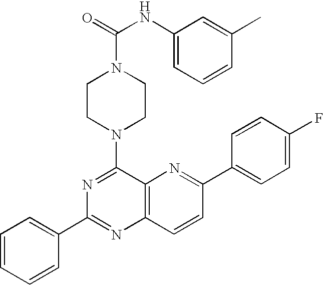

### EXAMPLE 534

### Synthesis of 2-chloromethyl-4-[N-(tert-butoxycarbonyl)piperazin-1-yl]-6-(4-fluorophenyl)-pyrido[3,2-d]pyrimidine

To a solution of 2-chloromethyl-6-(4-fluorophenyl)-pyrido[3,2-d]pyrimidin-4(3H)-one (2.9 mmol, 850 mg) and N,N-diisopropylethylamine (3.2 mmol, 552 μL) in toluene (40 mL) was added POCl₃ (4.1 mmol, 376 μL). The reaction mixture was stirred at 110° C. for 3 hours, cooled down to room temperature and extracted with dichloromethane and 2N hydrogen chloride. The organic phase was dried over magnesium sulfate and reduced in vacuo. The residue was dissolved in 1,4-dioxane (80 mL) and tert-butyl 1-piperazinecarboxylate (3.2 mmol, 601 mg), followed by N,N-diisopropylethylamine (6.5 mmol, 1.1 mL) were added. The reaction mixture was stirred at room temperature for 16 hours whereupon the solvent was evaporated in vacuo. The residue was purified by means of silica gel column chromatography (the mobile phase being a dichloromethane/methanol mixture, in a ratio gradually ranging from 99.5:0.5 to 99:1) yielding the pure title compound (1.115 g, yield 83%) which was characterized by its mass spectrum as follows: MS (m/z): 458 ([M+H]⁺, 100).

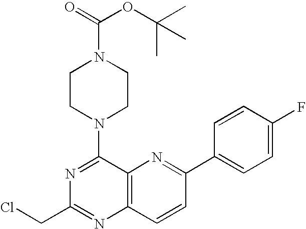

### EXAMPLES 535 TO 536

### Synthesis of 2-chloromethyl-4-[N-(3-methylphenyl-carbamoyl)piperazin-1-yl]-6-(4-fluorophenyl)-pyrido[3,2-d]pyrimidine and 2-chloro-methyl-4-[N-(3-methylbenzylcarbamoyl)piperazin-1-yl]-6-(4-fluorophenyl)-pyrido[3,2-d pyrimidine analogues

To a solution of 2-chloromethyl-4-[N-(tert-butoxycarbonyl)piperazin-1-yl]-6-(4-fluorophenyl)-pyrido[3,2-d]pyrimidine (0.44 mmol, 200 mg) in dichloromethane (20 mL) was added trifluoroacetic acid (20 mL). The reaction mixture was stirred at room temperature for 1 hour after which the solvents were evaporated in vacuo. The residue was dissolved in dichloromethane (20 mL) and N,N-diisopropylethylamine (7.9 mmol, 1.3 mL) followed by the appropriate isocyanate (0.87 mmol) were added. The mixture was stirred at room temperature for 3 hours, the solvents were evaporated in vacuo and the residue was purified by silica gel column chromatography (the mobile phase being a dichloromethane/methanol mixture, in a ratio gradually ranging from 99:1 to 97:3) yielding the corresponding, pure title compounds (yields ranging from 81% to 87%) which were characterised as follows.

### EXAMPLE 535

### 2-chloromethyl-4-[N-(3-methylphenylcarbamoyl)piperazin-1-yl]-6-(4-fluorophenyl)-pyrido[3,2-d]pyrimidine

This compound was obtained from m-tolyl isocyanate in 87% yield and was characterized by its mass spectrum as follows: MS (m/z): 491 ([M+H]⁺, 100).

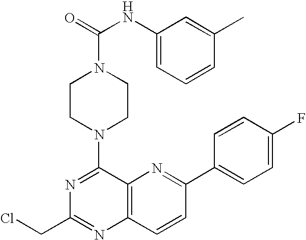

### EXAMPLE 536

### 2-chloromethyl-4-[N-(3-methylbenzylcarbamoyl)piperazin-1-yl]-6-(4-fluorophenyl)-pyrido[3,2-d]pyrimidine

This compound was obtained from 3-methylbenzyl isocyanate in 81% yield and was characterized by its mass spectrum as follows: MS (m/z): 505 ([M+H]⁺, 100).

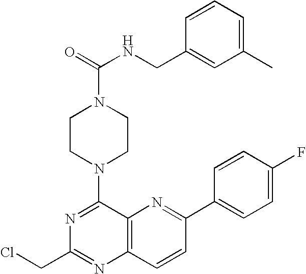

### EXAMPLES 537 TO 539

### Synthesis of 2-(N-alkylamino)methyl-4-[N-(3-methyl-phenylcarbamoyl)piperazin-1-yl]-6-(4-fluorophenyl)-pyrido[3,2-d]pyrimidine analogues

To a solution of 2-chloromethyl-4-[N-(3-methylphenylcarbamoyl)piperazin-1-yl]-6-(4-fluorophenyl)-pyrido[3,2-d]pyrimidine (0.10 mmol, 50 mg) in DMF (1 mL) was added the appropriate amine. The reaction mixture was stirred at room temperature for 20 hours whereupon the solvent was evaporated in vacuo. The residue was purified by preparative liquid chromatography (Waters Delta 600, XBridge™ Prep C18 5 μm 19×150 mm, using a gradient of water/acetonitrile (0.1% triethylamine) as mobile phase) yielding the corresponding pure compounds (yields ranging from 23% to 69%) which were characterised as follows.

### EXAMPLE 537

### 2-(N,N-dimethylamino)methyl-4-[N-(3-methylphenylcarbamoyl)piperazin-1-yl]-6-(4-fluorophenyl)-pyrido[3,2-d]pyrimidine

This compound was obtained from dimethylamine (40% in water) in 69% yield. and was characterized by its mass spectrum as follows: MS (m/z): 500 ([M+H]⁺, 100).

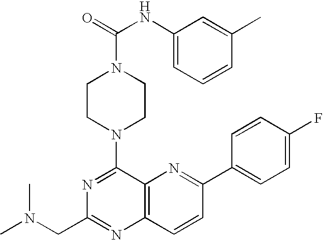

### EXAMPLE 538

### 2-(N-methoxyethylamino)methyl-4-[N-(3-methylphenylcarbamoyl)piperazin-1-yl-6-(4-fluorophenyl)-pyrido[3,2-d]pyrimidine

This compound was obtained from methoxyethylamine in 24% yield and was characterized by its mass spectrum as follows: MS (m/z): 530 ([M+H]⁺, 100).

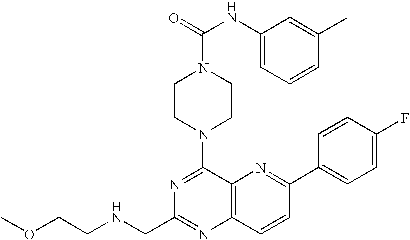

### EXAMPLE 539

### 2-(N-cyclopropylamino)methyl-4-[N-(3-methylphenylcarbamoyl)piperazin-1-yl]-6-(4-fluorophenyl)-pyrido[3,2-d]pyrimidine

This compound was obtained from cyclopropylamine and was characterized by its mass spectrum as follows: MS (m/z): 512 ([M+H]⁺, 100).

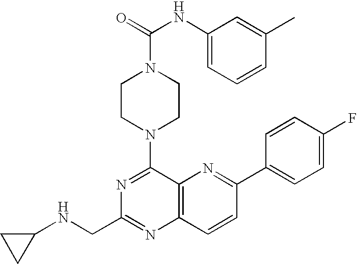

### EXAMPLE 540

### Synthesis of 2-amino-4-(N-piperazin-1-yl)-6-chloro-pyrido(3,2-d)pyrimidine

To a solution of 2-amino-6-chloro-pyrido[3,2-d]pyrimidin-4(3H)-one (example 20, 100 mg, 0.51 mmol) in DMF (20 ml) was added 1,8-diazabicyclo[5.4.0]undec-7-ene (DBU, 115 μl, 0.76 mmol), benzotriazol-1-yloxytris(dimethylamino)-phosphonium hexafluorophosphate (BOP, 0.66 mmol, 292 mg) and piperazine (1.53 mmol, 131 mg). The solution was stirred for 3 hours at room temperature. The solvents were evaporated in vacuo and the crude residue was purified by flash chromatography, the mobile phase being a mixture of methanol and dichloromethane (in a ratio of 3:97 with 0.5% aq. NH₃ solution), yielding the title compound as a white powder (85 mg, 63%) which was characterised as follows: MS (m/z): 266 ([M+H]⁺, 100).

### EXAMPLE 541

### Synthesis of 2-amino-4-[(3-methylphenyl carbamoyl)-piperazin-1-yl]-6-chloro-pyrido[3,2-d]pyrimidine

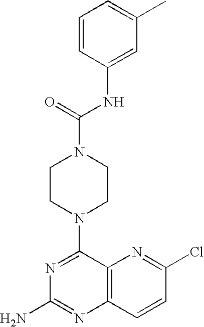

To a solution of 2-amino-4-(N-piperazin-1-yl)-6-chloro-pyrido(3,2-d)pyrimidine (85 mg, 0.32 mmol) in DMF (10 ml) was added m-tolyl isocyanate (0.35 mmol, 46 μl). The reaction was stirred at room temperature overnight. The solvents were evaporated and the crude residue was further purified by flash chromatography, the mobile phase being a mixture of methanol/dichloromethane (in a ratio ranging from 2:98 to 3:97), yielding the pure title compound as a white solid (91 mg, 72%) which was characterised as follows MS (m/z): 398 ([M+H]⁺, 100).

### EXAMPLES 542 TO 551

### Synthesis of 2-amino-4-[(3-methylphenyl carbamoyl)-piperazin-1-yl]-6-aryl-pyrido[3,2-d]pyrimidine analogues

To a solution of 2-amino-4-[(3-methylphenyl carbamoyl)-piperazin-1-yl]-6-chloro-pyrido[3,2-d]pyrimidine (70 mg, 0.18 mmol) in dioxane (15 ml) and water (3 ml) was added potassium carbonate (0.53 mmol, 72 mg), the appropriate arylboronic or heteroarylboronic acid (0.23 mmol) and tetrakis(triphenylphosphine)palladium (8.8 μmol, 10 mg). The reaction was refluxed for 2 hours. The solvents were evaporated and the crude residue was purified by silica gel flash chromatography, the mobile phase being a mixture of methanol and dichloromethane (in a ratio gradually ranging from 2:98 to 3:97), yielding the pure title compounds in yields ranging from 64 to 75% which were characterised as follows.

### EXAMPLE 542

### 2-amino-4-[(3-methylphenyl carbamoyl)-piperazin-1-yl]-6-(4-pyridyl)-pyrido[3,2-d]pyrimidine

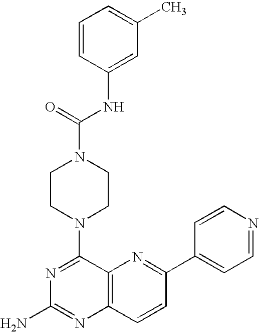

This compound was obtained from 4-pyridylboronic acid and was characterised as follows MS (m/z): 441 ([M+H]⁺, 100)

### EXAMPLE 543

### 2-amino-4-[(3-methylphenyl carbamoyl)-piperazin-1-yl]-6-(4-cyano-phenyl)-pyrido[3,2-d]pyrimidine

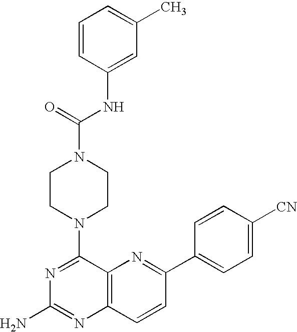

This compound was obtained from 4-cyanophenylboronic acid and was characterised as follows: MS (m/z): 465 ([M+H]⁺, 100).

### EXAMPLE 544

### 2-amino-4-[(3-methylphenyl carbamoyl)-piperazin-1-yl]-6-(3-fluoro-phenyl)-pyrido[3,2-d]pyrimidine

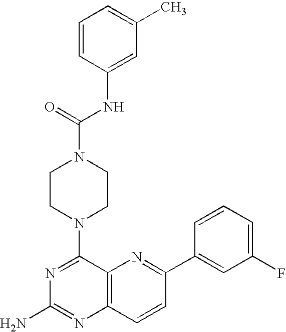

This compound was obtained from 3-fluorophenylboronic acid and was characterised as follows:

MS (m/z): 458 ([M+H]⁺, 100)

### EXAMPLE 545

### 2-amino-4-[(3-methylphenyl carbamoyl)-piperazin-1-yl]-6-(3-cyanophenyl)-pyrido[3,2-d]pyrimidine

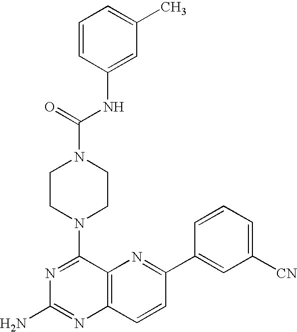

This compound was obtained from 3-cyanophenylboronic acid and was characterised as follows: MS (m/z): 465 ([M+H]⁺, 100)

### EXAMPLE 546

### 2-amino-4-[(3-methylphenyl carbamoyl)-piperazin-1-yl]-6-(2-cyano-phenyl)-pyrido[3,2-d]pyrimidine

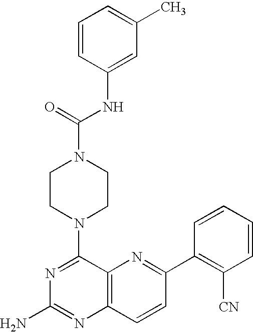

This compound was obtained from 2-cyanophenylboronic acid and was characterised as follows: MS (m/z): 465 ([M+H]⁺, 100)

### EXAMPLE 547

### 2-amino-4-[(3-methylphenyl carbamoyl)-piperazin-1-yl]-6-(2-fluoro-phenyl)-pyrido[3,2-d]pyrimidine

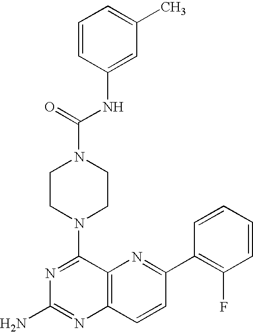

This compound was obtained from 2-fluorophenylboronic acid and was characterised as follows: MS (m/z): 457 ([M+H]⁺, 100)

### EXAMPLE 548

### 2-amino-4-[(3-methylphenyl carbamoyl)-piperazin-1-yl]-6-(4-trifluoromethylphenyl)-pyrido[3,2-d]pyrimidine

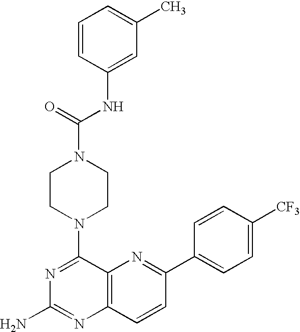

This compound was obtained from 4-trifluoromethylphenylboronic acid and was characterised as follows:

MS (m/z): 508 ([M+H]⁺, 100).

### EXAMPLE 549

### 2-amino-4-[(3-methylphenyl carbamoyl)-piperazin-1-yl]-6-(2-chlorophenyl)-pyrido[3,2-d]pyrimidine

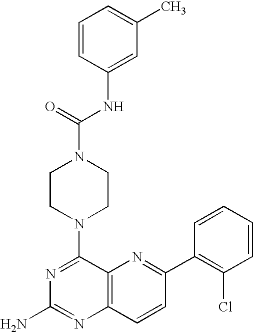

This compound was obtained from 2-chlorophenylboronic acid and was characterised as follows:

MS (m/z): 474 ([M+H]⁺, 100)

### EXAMPLE 550

### 2-amino-4-[(3-methylphenyl carbamoyl)-piperazin-1-yl]-6-(2,6-dimethyl-phenyl)-pyrido[3,2-d]pyrimidine

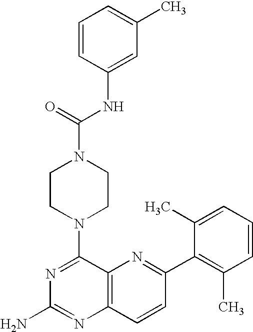

This compound was obtained from 2,6-dimethylphenylboronic acid and was characterised as follows:

MS (m/z): 467 ([M+H]⁺, 100).

### EXAMPLE 551

### 2-amino-4-[(3-methylphenyl carbamoyl)-piperazin-1-yl]-6-(2,4,6-trifluoro-phenyl)-pyrido[3,2-d]pyrimidine

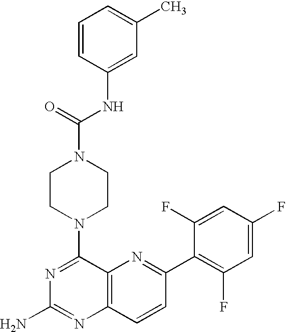

This compound was obtained from 2,4,6-trifluorophenylboronic acid and was characterised as follows:

MS (m/z): 494 ([M+H]⁺, 100).

### EXAMPLE 552

### Synthesis of 4-[(3-methylphenyl carbamoyl)-piperazin-1-yl]-6-chloro-Pyrido[3,2-d]pyrimidine

To a solution of 4-(N-piperazin-1-yl)-6-chloro-pyrido[3,2-d]-pyrimidine (example 65; 450 mg; 1.8 mmol) in dichloromethane (30 ml) was added m-tolyl isocyanate (1 ml). The reaction mixture was stirred for 16 hours. The solvents were evaporated in vacuo. The crude residue was purified by silica gel flash chromatography, the mobile phase being a mixture of dichloromethane and methanol (in a ratio of 95:5), yielding the title compound as a white solid (598 mg, 87%) and was characterised as follows:

MS (m/z): 383 ([M+H]⁺, 100).

### EXAMPLES 553-563

### Synthesis of 4-[(3-methylphenyl carbamoyl)-piperazin-1-yl]-6-aryl Pyrido[3,2-d]pyrimidine analogues

A solution of 4-[(3-methylphenyl carbamoyl)-piperazin-1-yl]-6-chloro-pyrido[3,2-d]pyrimidine (0.2 mmol, 77 mg), the appropriate arylboronic or heteroarylboronic acid (0.3 mmol) and potassium fluoride (46 mg, 0.8 mmol) in dioxane (8 ml) and water (2 ml) was degassed for 30 minutes. Then, tetrakis(triphenylphosphine)palladium (30 mg) was added and the reaction mixture was refluxed for 3 hours. The solvents were evaporated and the residue was further purified by flash chromatography on silica, the mobile phase being a mixture of methanol and dichloromethane (in a ratio of 4:96), yielding the title compounds as white powders, in yields ranging from 50 to 70%, which were characterised as follows:

### EXAMPLE 553

### 4-[(3-methylphenyl carbamoyl)-piperazin-1-yl]-6-(4-cyanophenyl)pyrido[3,2-d]pyrimidine

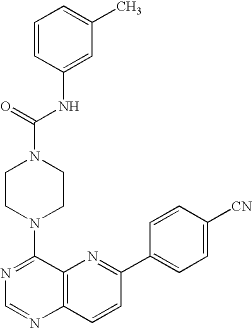

This compound was obtained from 4-cyanophenylboronic acid and was characterised as follows: MS (m/z): ([M+H]⁺, 100).

### EXAMPLE 554

### 4-[(3-methylphenyl carbamoyl)-piperazin-1-yl-6-(3-cyanophenyl)pyrido[3,2-d]pyrimidine

This compound was obtained from 3-cyanophenylboronic acid and was characterised as follows: MS (m/z): ([M+H]⁺, 100).

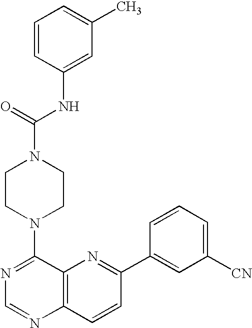

### EXAMPLE 555

### 4-[(3-methylphenyl carbamoyl)-piperazin-1-yl]-6-(3-methyl-4-fluoro-phenyl)pyrido[3,2-d]pyrimidine

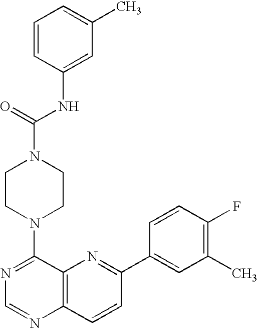

This compound was obtained from 3-methyl-4-fluoro-phenylboronic acid and was characterised as follows:

MS (m/z) 457 ([M+H]⁺, 100).

### EXAMPLE 556

### 4-[(3-methylphenyl carbamoyl)-piperazin-1-yl]-6-(2,4-difluorophenyl) pyrido[3,2-d]pyrimidine

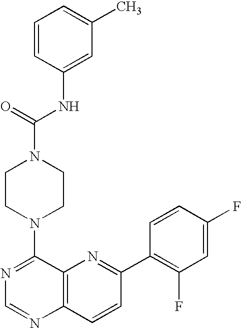

This compound was obtained from 2,4-difluorophenylboronic acid and was characterised as follows:

MS (m/z): 461 ([M+H]⁺, 100).

### EXAMPLE 557

### 4-[(3-methylphenyl carbamoyl)-piperazin-1-yl]-6-(2-cyanophenyl) pyrido[3,2-d]pyrimidine

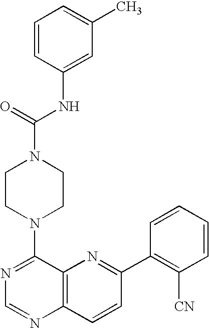

This compound was obtained from 2-cyanophenylboronic acid and was characterised as follows:

MS (m/z): 449 ([M+H]⁺, 100).

### EXAMPLE 558

### 4-[(3-methylphenyl carbamoyl)-piperazin-1-yl]-6-(4-fluorophenyl) pyrido[3,2-d]pyrimidine

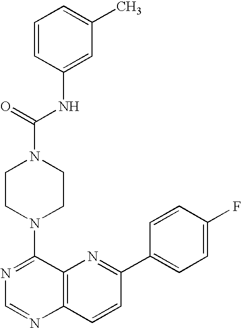

This compound was obtained from 4-fluorophenylboronic acid and was characterised as follows: MS (m/z): 443 ([M+H]⁺, 100)

### EXAMPLE 559

### 4-[(3-methylphenyl carbamoyl)-piperazin-1-yl]-6-(3-fluoro-4-ethoxyphenyl)pyrido[3,2-d]pyrimidine

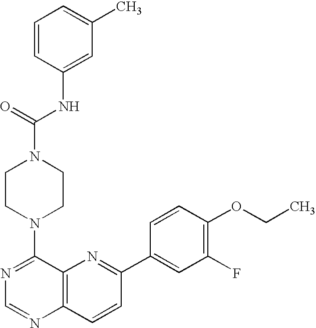

This compound was obtained from 3-fluoro-4-ethoxy-phenylboronic acid and was characterised as follows: MS (m/z): 487 ([M+H]⁺, 100)

### EXAMPLE 560

### 4-[(3-methylphenylcarbamoyl)-piperazin-1-yl]-6-(3-chloro-4-ethoxy-phenyl)pyrido[3,2-d]pyrimidine

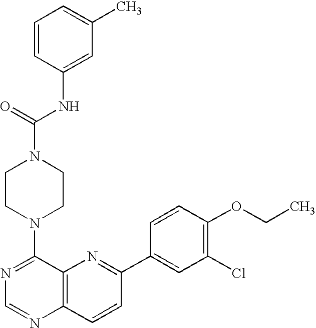

This compound was obtained from 3-chloro-4-ethoxyphenyl boronic acid and was characterised as follows: MS (m/z): 503 ([M+H]⁺, 100)

### EXAMPLE 561

### 4-[(3-methylphenyl carbamoyl)-piperazin-1-yl]-6-(3,4,5-trifluorophenyl)-pyrido[3,2-d]pyrimidine

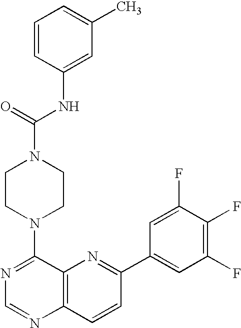

This compound was synthesized from 3,4,5-trifluorophenylboronic acid and was characterised as follows:

MS (m/z): 479 ([M+H]⁺, 100).

### EXAMPLE 562

### 4-[(3-methylphenyl carbamoyl)-piperazin-1-yl]-6-(2-thienyl)pyrido[3,2-d]pyrimidine

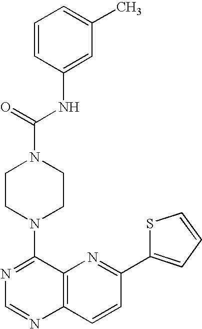

This compound was obtained from 2-thienylboronic acid and was characterised as follows:

MS (m/z): 431 ([M+H]⁺, 100).

### EXAMPLE 563

### 4-[(3-methylphenyl carbamoyl)-piperazin-1-yl]-6-(2-furanyl)pyrido[3,2-d]pyrimidine

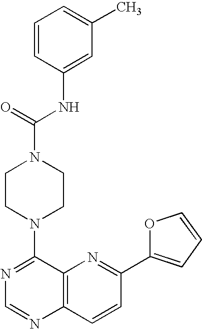

This compound was obtained from 2-furylboronic acid and was characterised as follows:

MS (m/z): 414 ([M+H]⁺, 100).

### EXAMPLE 564

### Synthesis 2,4-dihydroxy-6-(4-fluorophenyl)-pyrido[3,2-d]pyrimidine

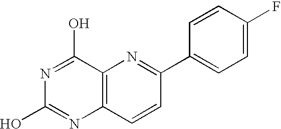

A mixture of 3-amino-6-(4-fluorophenyl)-pyridine-2-carboxylic acid amide (0.92 g, 4.0 mmol) and triphosgene (0.60 g, 2.0 mmol) in 10 ml dioxane was heated under reflux for 2 h. After cooling to room temperature, the precipitate was collected by filtration and washed with diethyl ether. The title compound was obtained (0.98 g, 95%) as a yellowish solid and was characterised as follows:

MS (m/z): 258.2 ([M+H]⁺, 100).

### EXAMPLE 565

### Synthesis of 2,4-dichloro-6-(4-fluorophenyl)-pyrido[3,2-d]pyrimidine

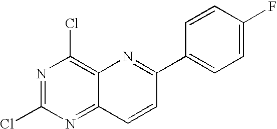

A suspension of 2,4-dihydroxy-6-(4-fluorophenyl)-pyrido[3,2-d]pyrimidine (0.52 g, 2.0 mmol) in phosphorus oxychloride (10 ml) and diisopropylethylamine (1 ml) was refluxed for 6 hours. After concentration under reduced pressure, the residue was extracted with dichloromethane (100 ml) and washed with ice water till pH=6-7. The combined organic layers were dried over MgSO₄, filtered and concentrated under reduced pressure. The crude residue was purified by silica gel flash chromatography, the mobile phase being a mixture of methanol and dichloromethane, in a ratio of 1:50, yielding the title compound as a white solid (400 mg, 68%) and was characterised as follows:

MS (m/z): 295.2 ([M+H]⁺, 100).

### EXAMPLE 566

### Synthesis of 2-chloro-6-(4-fluorophenyl)-4-(4-Boc-piperazino)pyrido[3,2-d]pyrimidine

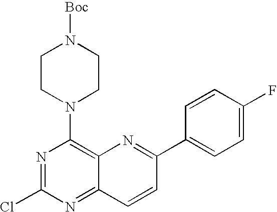

A mixture of 2,4-dichloro-6-(4-fluorophenyl)-pyrido[3,2-d]pyrimidine (0.35 g, 1.2 mmol) and 1-Boc-piperazine (0.28 g, 1.5 mmol) in dioxane (20 ml) was stirred at room temperature for 1 hour. After concentration under reduced pressure, the residue was purified by silica gel flash chromatography, the mobile phase being a mixture of methanol and dichloromethane (in a ratio of 1:100), yielding the title compound as a white solid (0.52 g, 98%) which was characterised as follows:

MS (m/z): 444.2 ([M+H]⁺, 100).

### EXAMPLE 567

### Synthesis of 6-(4-fluorophenyl)-4-(4-Boc-piperazino)-2-pyrrolidino-Pyrido[3,2-d]pyrimidine

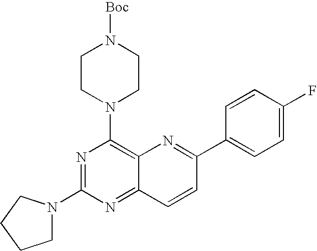

A mixture of 2-chloro-6-(4-fluorophenyl)-4-(4-Boc-piperazino)-pyrido[3,2-d]pyrimidine (133 mg, 0.3 mmol) and pyrrolidine[2.0 ml) in dioxane (10 ml) was heated under reflux for 4 hours. After concentration under reduced pressure, the residue was purified by silica gel flash chromatography, the mobile phase being a mixture of methanol and dichloromethane, in a ratio of 1:25, yielding the title compound as a yellow solid (140 mg, 97%) which was characterised as follows:

MS (m/z): 479.1 ([M+H]⁺, 100)

### EXAMPLE 568

### Synthesis of 2-cyclopentylamino-6-(4-fluorophenyl)-4-(4-Boc-piperazino)-pyrido[3,2-d]pyrimidine

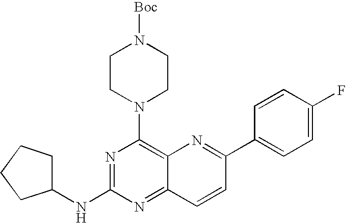

A similar procedure as for the synthesis of the compound of previous example was followed, using cyclopentylamine instead of pyrrolidine. The pure title compound was isolated in 95% yield as a yellow solid which was characterised as follows:

MS (m/z): 493.1 ([M+H]⁺, 100)

### EXAMPLE 569

### Synthesis of 2-methylamino-6-(4-fluorophenyl)-4-(4-Boc-piperazino)-pyrido[3,2-d]pyrimidine and 2,4-dimethylamino-6-(4-fluorophenyl)pyrido[3,2-d]pyrimidine

A mixture of 2-chloro-6-(4-fluorophenyl)-4-(4-Boc-piperazino)-pyrido[3,2-d]pyrimidine (400 mg, 0.9 mmol) and methylamine in THF (2 ml, 8 mmol) was heated in a sealed tube at 90° C. for 3 hours. After cooling to room temperature, the solvents were removed under reduced pressure. The residue was purified by silica gel flash chromatography, the mobile phase being a mixture of methanol and dichloromethane (in a ratio of 1:20), yielding two compounds:

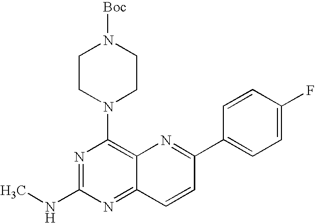

2-methylamino-6-(4-fluorophenyl)-4-(4-Boc-piperazino)-pyrido[3,2-d]pyrimidine(237 mg, 60%), which was characterised as follows:

MS (m/z): 439.1 ([M+H]⁺, 100); and

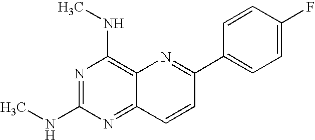

2,4-dimethylamino-6-(4-fluorophenyl)pyrido[3,2-d]pyrimidine (89 mg, 35%) which was characterised as follows:

MS (m/z): 284.2 ([M+H]⁺, 100)

### EXAMPLE 570

### Synthesis of 6-(4-fluorophenyl)-4-[(3-methylphenyl carbamoyl)-piperazin-1-yl]-2-pyrrolidino-pyrido[3,2-d]pyrimidine

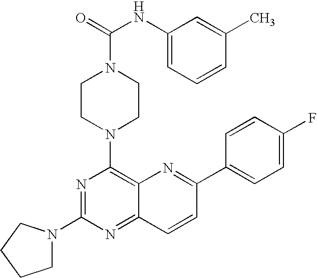

To a solution of 2-pyrrolidino-6-(4-fluorophenyl)-4-(4-Boc-piperazino)-pyrido[3,2-d]pyrimidine (30 mg, 0.06 mmol) in dichloromethane (1 ml), was added trifluoroacetic acid (1 ml). The resulting mixture was stirred at room temperature for 30 minutes. After concentration under reduced pressure, the residue was dissolved in dichloromethane (5 ml). Then, diisopropylethylamine (1 ml) and m-tolyl isocyanate (1 ml) were added respectively. The mixture was stirred at room temperature for 30 min. The solvents were removed under reduced pressure and residue was purified by silica gel flash chromatography, the mobile phase being a mixture of methanol and dichloromethane (in a ratio of 1:30), yielding the title compound as a white solid (30 mg, 98%) which was characterised as follows:

MS (m/z): 512.2 ([M+H]⁺, 100)

### EXAMPLE 571

### Synthesis of 2-cyclopentylamino-6-(4-fluorophenyl)-4-[(3-methylphenyl carbamoyl)-piperazin-1-yl]-pyrido[3,2-d]pyrimidine

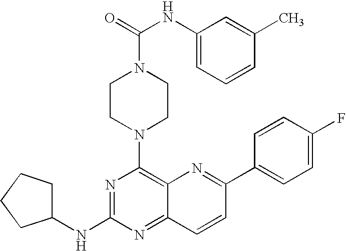

This compound was synthesized from 2-cyclopentylamino-6-(4-fluorophenyl)-4-(4-Boc-piperazino)-pyrido[3,2-d]pyrimidine using the procedure from previous example, yielding the title compound as a white solid in 95% yield which was characterised as follows:

MS (m/z): 526.2 ([M+H]⁺, 100).

### EXAMPLE 572

### Synthesis of 2-pyrrolidino-4-[N-(4-chlorophenoxyacetyl)-piperazin-1-yl]-6-(4-fluorophenyl)-pyrido[3,2-d]pyrimidine

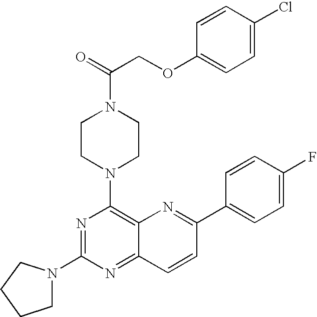

To a solution of 2-pyrrolidino-6-(4-fluorophenyl)-4-(4-Boc-piperazino)-pyrido[3,2-d]pyrimidine (25 mg, 0.05 mmol) in dichloromethane (1 ml), was added trifluoroacetic acid (1 ml) was added. The resulting mixture was stirred at room temperature for 30 minutes. After concentration under reduced pressure, the residue was dissolved in dichloromethane (5 ml). Then, diisopropylethylamine (1 ml) and 4-chlorophenoxyacetyl chloride (0.6 mmol) were added respectively. The mixture was stirred at room temperature for 30 minutes. The solvents were removed under reduced pressure and residue was purified by silica gel flash chromatography, the mobile phase being a mixture of methanol and dichloromethane (in a ratio of 1:30), yielding the title compound as a white solid (25 mg, 89%) which was characterised as follows:

MS (m/z): 561.2 ([M+H]⁺, 100)

### EXAMPLE 573

### Synthesis of 2-cyclopentylamino-6-(4-fluorophenyl)-4-[N-(4-chlorophenoxyacetyl)-piperazin-1-yl]-pyrido[3,2-d]pyrimidine

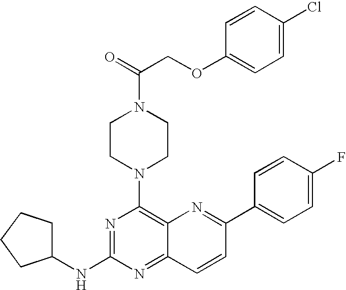

This compound was synthesized from 2-cyclopentylamino-6-(4-fluorophenyl)-4-(4-Boc-piperazino)-pyrido[3,2-d]pyrimidine using the procedure described for example 572. The title compound isolated in 91% yield was characterised as follows:

MS (m/z): 547.2 ([M+H]⁺, 100).

### EXAMPLE 574

### Synthesis of 2-methylamino-4-[N-(4-chlorophenoxyacetyl)-piperazin-1-yl]-6-(4-fluorophenyl)-pyrido[3,2-d]pyrimidine

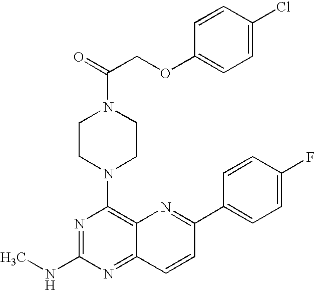

This compound was synthesized from 2-methylamino-6-(4-fluorophenyl)-4-(4-Boc-piperazino)-pyrido[3,2-d]pyrimidine using the procedure described for example 572, yielding the title compound as a yellow solid (75% yield) which was characterised as follows:

MS (m/z): 507.1 ([M+H]⁺, 100).

### EXAMPLE 575

### Synthesis of 2-amino-4-[(R)-3-Boc-amino-pyrrolidin-1-yl]-6-(4-fluoro-phenyl)-pyrido(3,2-d)pyrimidine

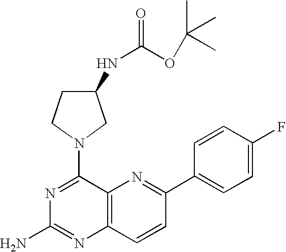

To a solution of 2-acetylamino-4-(1,2,4-triazolyl)-6-(4-fluorophenyl)-pyrido(3,2-d)pyrimidine (163 mg, 0.5 mmol) in dioxane (20 ml) was added [(R)-3-Boc-amino-pyrrolidine (93 mg, 0.5 mmol). The reaction mixture was stirred at 50° C. overnight, yielding crude 2-acetylamino-4-[(R)-3-Boc-amino-pyrrolidin-1-yl]-6-(4-fluorophenyl)-pyrido(3,2-d)pyrimidine. In order to deprotect the acetyl group, the solvents were evaporated in vacuo and the crude residue (containing crude 2-acetylamino-4-[(R)-3-Boc-amino-pyrrolidin-1-yl]-6-(4-fluorophenyl)-pyrido(3,2-d)pyrimidine) was redissolved in a mixture of dichloromethane and ethanol (in a ratio of 80:20, 10 ml). A sodium ethoxide solution (0.2 N solution) was added till pH=12 and the reaction mixture was stirred overnight at room temperature. The solvents were evaporated in vacuo and the crude residue was purified by preparative thin layer chromatography on silica, the mobile phase being a mixture of methanol and dichloromethane (in a ratio of 10:90), yielding the title compound as a white powder (89 mg, 42%) which was characterised as follows:

MS (m/z): 425 ([M+H]⁺, 100).

### EXAMPLE 576

### Synthesis of 2-amino-4-[(S)-3-Boc-amino-pyrrolidin-1-yl]-6-(4-fluorophenyl)-pyrido(3,2-d)pyrimidine

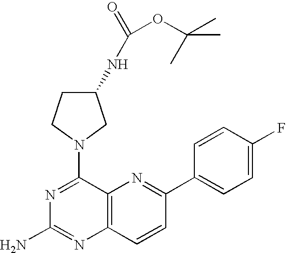

This compound was synthesized in a similar way as described for example 575, using (S)-3-Boc-amino-pyrrolidine as reagent, and was characterised as follows:

MS (m/z): 425 ([M+H]⁺, 100).

### EXAMPLE 577

### Synthesis of 2-amino-4-[(S)-1-Boc-amino-pyrrolidin-3-yl]-6-(4-fluorophenyl)-pyrido(3,2-d)pyrimidine

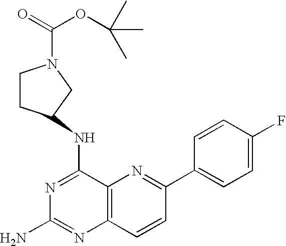

This compound was synthesized using the procedure of example 575, using (S)-1-Boc-3-aminopyrrolidine as a reagent and was characterised as follows:

MS (m/z): 425 ([M+H]⁺, 100)

### EXAMPLE 578

### Synthesis of 2-amino-4-[3-(S)-3-methylphenylureylpyrrolidin-1-yl]-pyrido(3,2-d)pyrimidine

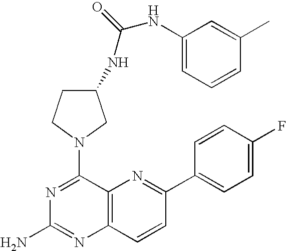

To a solution of 2-amino-4-[(S)-3-Boc-amino-pyrrolidin-1-yl]-6-(4-fluorophenyl)-pyrido(3,2-d)pyrimidine from example 576 (100 mg, 0.24 mmol) in dichloromethane (10 ml) was added a mixture of dichloromethane/trifluoroacetic acid (3 ml, 1:1). The reaction mixture was stirred for 30 minutes at room temperature. The solvents were evaporated in vacuo. The crude residue was redissolved in dichloromethane (5 ml) and diisopropylethylamine (30 μl) and m-tolylisocyanate (30 μl) were added. The reaction was stirred for 2 hours at room temperature. The solvents were evaporated in vacuo and the crude residue was further purified by thin layer preparative thin layer chromatography, the mobile phase being a mixture of methanol and dichloromethane (in a ratio of 10:90), yielding the pure title compound as a white powder (66 mg, 61%) which was characterised as follows:

MS (m/z): 458 ([M+H]⁺, 100).

### EXAMPLE 579

### Synthesis of 2-amino-4-[3-(R)-3-methylphenylureylpyrrolidin-1-yl]-pyrido(3,2-d)pyrimidine

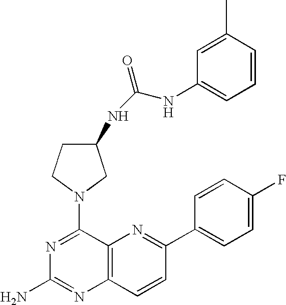

This compound was synthesized using similar methods as described in example 578, using 2-amino-4-[(R)-3-Boc-amino-pyrrolidin-1-yl]-6-(4-fluoro-phenyl)-pyrido(3,2-d)pyrimidine from example 575 as the starting material, and was characterised as follows:

MS (m/z): 458 ([M+H]⁺, 100).

### EXAMPLE 580

### Synthesis of 2-amino-4-[1-(3-methylphenylcarbamoyl)-(S)-amino-pyrrolidin-3-yl]-6-(4-fluorophenyl)-pyrido(3,2-d)pyrimidine

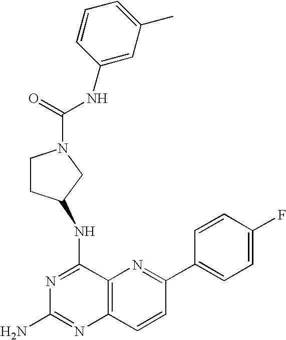

This compound was synthesized using similar methods as described in example 578, using 2-amino-4-[(S)-3-Boc-amino-pyrrolidin-1-yl]-6-(4-fluorophenyl)-pyrido(3,2-d)pyrimidine from example 577 as the starting material, and was characterised as follows:

MS (m/z): 458 ([M+H]⁺, 100).

### EXAMPLE 581

### Synthesis of 2-amino-4-(N-piperidine-4-carboxylic acid m-tolylamide)-6-(4-fluorophenyl)-pyrido(3,2-d)-pyrimidine

Step (a): Synthesis of m-tolylcarbamoyl-piperidine-1-carboxylic acid tert-butyl ester:

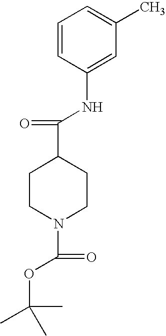

To a solution of N-Boc-piperidine-4-carboxylic acid (1 g, 4.36 mmol) in dry dichloromethane (15 ml) was added 1-hydroxybenzotriazole (HOBT, 600 mg, 4.36 mmol) and dicyclohexylcarbodiimide (900 mg, 4.36 mmol). This solution was stirred for 15 minutes. Then, m-toluidine (4.36 mmol, 0.45 ml) was added and the reaction was stirred overnight at room temperature. The solvents were evaporated in vacuo and the crude residue was redissolved in ethylacetate (10 ml). The precipitate was filtered off and further purified by silica gel flash chromatography, the mobile phase being a mixture of methanol and dichloromethane (in a ratio of 2:98) yielding the pure title compound (1.15 g, 83%) which was characterised as follows:

MS (m/z): 319 ([M+H]⁺, 100)

Step (b): Synthesis of Piperidine-4-carboxylic acid m-tolylamide:

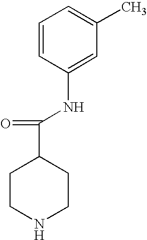

To a solution of m-tolylcarbamoyl-piperidine-1-carboxylic acid tert-butyl ester (1 mmol, 438 mg) in dichloromethane (20 ml) was added a mixture of dichloromethane and trifluoroacetic acid (5 ml, ratio 1:1). The reaction mixture was stirred at room temperature for 90 minutes. The solvents were evaporated in vacuo. The residue was redissolved in dioxane and a few drops of triethylamine were added. The solvents were evaporated and the residue was resuspended in diethyl ether. A white precipitate was formed, which was filtered off yielding the pure title compound (200 mg, 92%) which was characterised as follows:

MS (m/z): 219 ([M+H]⁺, 100).

Step (c): Synthesis of 2-amino-4-(piperidinyl-4-carboxylic acid m-tolylamide)-6-(4-fluorophenyl)-pyrido(3,2-d)-pyrimidine

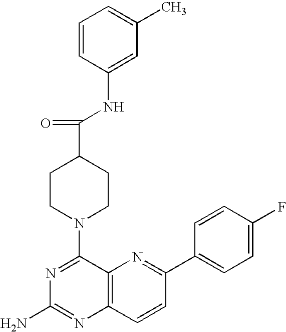

To a solution of 2-acetylamino-4-(1,2,4-triazolyl)-6-(4-fluorophenyl)-pyrido(3,2-d)pyrimidine (163 mg, 0.5 mmol) in dioxane (20 ml) was added piperidine-4-carboxylic acid m-tolylamide (93 mg, 0.5 mmol). The reaction mixture was stirred at 50° C. overnight, yielding crude 2-acetylamino-4-[piperidinyl-4-carboxylic acid m-tolylamide)-6-(4-fluorophenyl)-pyrido(3,2-d)pyrimidine. In order to deprotect the acetyl group, the solvents were evaporated in vacuo and the crude residue was redissolved in a mixture of dichloromethane and ethanol (in a ratio of 80:20, 10 ml). A sodium ethoxide solution (0.2 N solution) was added till pH=12 and the reaction mixture was stirred overnight at room temperature. The solvents were evaporated in vacuo and the crude residue was purified by preparative thin layer chromatography on silica, the mobile phase being a mixture of methanol and dichloromethane (in a ratio of 10:90), yielding the title compound as a white powder (72 mg, 32%) which was characterised as follows:

MS (m/z): 457 ([M+H]⁺, 100).

### EXAMPLE 582

### Synthesis of 2-amino-4-(4-Hydroxy-piperidine-1-carboxylic acid m-tolylamide)-6-(4-fluorophenyl)-pyrido(3,2-d)-pyrimidine

Step (a): Synthesis of 2-acetylamino-(4-hydroxy-N-Boc-piperidine)-6-(4-fluorophenyl)-pyrido(3,2-d)-pyrimidine

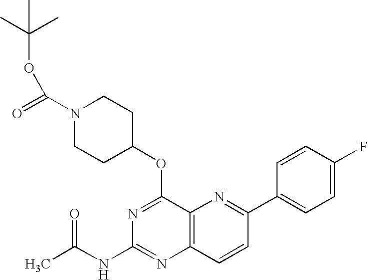

To a solution of N-Boc-4-hydroxy-piperidine (402 mg, 2 mmol) in dioxane (40 ml) was added NaH (70 mg of a 60% dispersion; 2.2 mmol). The solution was stirred for 30 minutes at room temperature. Then, 2-acetylamino-4-(1,2,4-triazolyl)-6-(4-fluorophenyl)-pyrido(3,2-d)pyrimidine (326 mg, 1 mmol) was added and the resulting solution was stirred at room temperature for 24 hours. The precipitate was filtered off, redissolved in dichloromethane and extracted several times with a 0.1 N HCl solution and water. The combined organic layers were dried over sodium sulfate, and evaporated in vacuo yielding the crude compound, which was used for further reaction without any purification, and was characterised as follows:

MS (m/z): 482 ([M+H]⁺, 100)

Step (b): 2-acetylamino-4-(Hydroxy-piperidine-1-carboxylic acid m-tolylamide)-6-(4-fluorophenyl)-pyrido(3,2-d)-pyrimidine

The crude residue obtained in step (a) was redissolved in dichloromethane (10 ml) and a mixture of dichloromethane and trifluoroacetic acid (1:1 ratio; 5 ml) was added. The reaction was stirred for 1 hour at room temperature. The solvents were evaporated yielding a crude residue, which was redissolved in dichloromethane (25 ml). Diisopropylethylamine (150 μl), and m-tolylisocyanate (100 μl) were added. The reaction was stirred for 4 hours at room temperature. The solvents were evaporated in vacuo and the residue was purified by preparative thin layer chromatography, the mobile phase being a mixture of methanol and dichloromethane (in a ratio of 10:90), yielding the pure title compound (87 mg, 14%) which was characterised as follows:

MS (m/z): 515 ([M+H]⁺, 100)

Step (c): 2-amino-4-[4-Hydroxy-piperidine-1-carboxylic acid m-tolylamide-6-(4-fluorophenyl)-pyrido(3,2-d)-pyrimidine

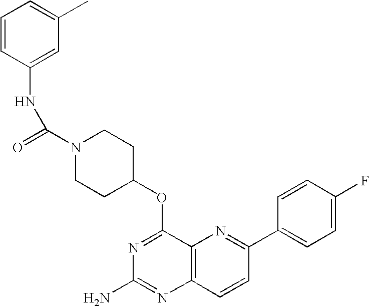

A solution of 2-acetylamino-4-(4-hydroxy-piperidine-1-carboxylic acid m-tolylamide)-6-(4-fluorophenyl)-pyrido(3,2-d)pyrimidine (50 mg, 0.097 mmol) in dioxane/1 M K₂CO₃ in water (ratio 80:20; 10 ml) was stirred for 48 hours at 80° C. and for 1 week at room temperature. The solvents were evaporated in vacuo and the crude residue was purified by preparative thin layer chromatography, the mobile phase being a mixture of methanol and dichloromethane (in a ratio of 10:90), yielding the pure title compound (42%, 19 mg) which was characterised as follows:

MS (m/z): 473 ([M+H]⁺, 100)

### EXAMPLE 583

### Biological activity of Pyrido[3,2-d]pyrimidine derivatives

Some of the pyrido[3,2-d]pyrimidine derivatives being described in the previous examples 381 to 582 have been tested for biological activities according to the methodology of example 319, in particular for their activity in the MLR assay.

The detailed nomenclature of these pyrido[3,2-d]pyrimidine derivatives is shown in the following table 4, which also shows their IC₅₀ values (expressed in μM) in the MLR assay of example 319.

TABLE 4

| ex. Nr. | compound name | MLR IC50 (μM) |
|---|---|---|
| 383 | 2-amino-4-[4-(2-naphthoxyacetyl)piperazin-1-yl]-6-(4- | 0.5 |
| | fluorophenyl)-pyrido[3,2-d]pyrimidine | |
| 384 | 2-amino-4-[4-(3-methylphenoxyacetyl)piperazin-1-yl]-6-(4- | 0.05 |
| | fluorophenyl)-pyrido[3,2-d]pyrimidine | |
| 385 | 2-amino-4-[4-(3-chlorophenoxyacetyl)piperazin-1-yl]-6-(4- | 0.09 |
| | fluorophenyl)-pyrido[3,2-d]pyrimidine | |
| 386 | 2-amino-4-[4-(2,4-dichlorophenoxyacetyl)piperazin-1-yl]-6- | 0.4 |
| | (4-fluorophenyl)-pyrido[3,2-d]pyrimidine | |
| 387 | 2-amino-4-[4-(4-fluorophenoxyacetyl)piperazin-1-yl]-6-(4- | 0.2 |
| | fluorophenyl)-pyrido[3,2-d]pyrimidine | |
| 388 | 2-amino-4-[4-(4-bromophenoxyacetyl)piperazin-1-yl]-6-(4- | 0.05 |
| | fluorophenyl)-pyrido[3,2-d]pyrimidine | |
| 392 | 2-amino-4-[4-(3,3-dimethylbutyryl)piperazin-1-yl]-6-(3,4- | 0.3 |
| | dimethoxyphenyl)-pyrido[3,2-d]pyrimidine | |
| 393 | 2-amino-4-[4-(2-propenoyl)piperazin-1-yl]-6-(3,4- | 0.4 |
| | dimethoxyphenyl)-pyrido[3,2-d]pyrimidine | |
| 396 | 2-amino-4-{4-[N-(tert-butoxycarbonyl)-glycyl]-piperazin-1-yl}- | 0.05 |
| | 6-(3,4-di-methoxyphenyl)-pyrido[3,2-d]pyrimidine | |
| 399 | 2-amino-4-[4-(4-chlorophenoxyacetyl)piperazin-1-yl]-6-(4- | 0.5 |
| | trifluoromethylphenyl)-pyrido-[3,2-d]pyrimidine | |
| 400 | 2-amino-4-[4-(4-chlorophenoxyacetyl)piperazin-1-yl]-6-(4- | 0.05 |
| | cyanophenyl)pyrido-[3,2-d]pyrimidine | |
| 401 | 2-amino-4-[4-(4-chlorophenoxyacetyl)piperazin-1-yl]-6-(3- | 0.3 |
| | fluorophenyl)pyrido-[3,2-d]pyrimidine | |
| 402 | 2-amino-4-[4-(4-chlorophenoxyacetyl)piperazin-1-yl]-6- | 0.03 |
| | (furan-3-yl)pyrido-[3,2-d]pyrimidine | |
| 403 | 2-amino-4-[4-(4-chlorophenoxyacetyl)piperazin-1-yl]-6- | 0.04 |
| | (thiophen-3-yl)pyrido-[3,2-d]pyrimidine | |
| 404 | 2-amino-4-[4-(4-chlorophenoxyacetyl)piperazin-1-yl]-6-(3,4- | 0.04 |
| | difluorophenyl)pyrido-[3,2-d]pyrimidine | |
| 405 | 2-amino-4-[4-(4-chlorophenoxyacetyl)piperazin-1-yl]-6-(4- | 0.05 |
| | chlorophenyl)pyrido-[3,2-d]pyrimidine | |
| 406 | 2-amino-4-[4-(4-chlorophenoxyacetyl)piperazin-1-yl]-6-(3- | 0.07 |
| | chlorophenyl)pyrido-[3,2-d]pyrimidine | |
| 407 | 2-amino-4-[4-(4-chlorophenoxyacetyl)piperazin-1-yl]-6- | 0.005 |
| | (pyridin-4-yl)pyrido-[3,2-d]pyrimidine | |
| 408 | 2-amino-4-[4-(4-chlorophenoxyacetyl)piperazin-1-yl]-6-(3- | 0.2 |
| | chloro-4-fluorophenyl)-pyrido-[3,2-d]pyrimidine | |
| 409 | 2-amino-4-[4-(4-chlorophenoxyacetyl)piperazin-1-yl]-6- | 0.005 |
| | (pyridin-3-yl)pyrido-[3,2-d]pyrimidine | |
| 410 | 2-amino-4-[4-(4-chlorophenoxyacetyl)piperazin-1-yl]-6-(2- | 0.009 |
| | methoxypyridin-5-yl)pyrido-[3,2-d]pyrimidine | |
| 411 | 2-amino-4-[4-(4-chlorophenoxyacetyl)piperazin-1-yl]-6-(3,5- | 0.034 |
| | dimethylisoxazol-4-yl)pyrido-[3,2-d]pyrimidine | |
| 412 | 2-amino-4-[4-(4-chlorophenoxyacetyl)piperazin-1-yl]-6- | 0.039 |
| | (indol-5-yl)pyrido-[3,2-d]pyrimidine | |
| 413 | 2-amino-4-[4-(4-chlorophenoxyacetyl)piperazin-1-yl]-6-(2- | >10 |
| | carboxythiophen-5-yl)pyrido-[3,2-d]pyrimidine | |
| 414 | 2-amino-4-[4-(4-chlorophenoxyacetyl)piperazin-1-yl]-6-(3- | 0.02 |
| | cyanophenyl)pyrido-[3,2-d]pyrimidine | |
| 415 | 2-amino-4-[4-(4-chlorophenoxyacetyl)piperazin-1-yl]-6-(4- | 0.006 |
| | hydroxyphenyl)pyrido-[3,2-d]pyrimidine | |
| 416 | 2-amino-4-[4-(4-chlorophenoxyacetyl)piperazin-1-yl]-6-(2- | 0.012 |
| | cyanophenyl)pyrido-[3,2-d]pyrimidine | |
| 417 | 2-amino-4-[4-(4-chlorophenoxyacetyl)piperazin-1-yl]-6-(4- | 0.028 |
| | (methanesulfonyl)-phenyl)pyrido-[3,2-d]pyrimidine | |
| 418 | 2-amino-4-[4-(4-chlorophenoxyacetyl)piperazin-1-yl]-6-(3- | 0.057 |
| | methoxyphenyl)pyrido-[3,2-d]pyrimidine | |
| 419 | 2-amino-4-[4-(4-chlorophenoxyacetyl)piperazin-1-yl]-6-(3- | 0.02 |
| | aminophenyl)pyrido-[3,2-d]pyrimidine | |
| 420 | 2-amino-4-[4-(4-chlorophenoxyacetyl)piperazin-1-yl]-6-(4- | 0.3 |
| | fluoro-3-methylphenyl)-pyrido-[3,2-d]pyrimidine | |
| 421 | 2-amino-4-[4-(4-chlorophenoxyacetyl)piperazin-1-yl]-6- | 0.049 |
| | phenylpyrido-[3,2-d]pyrimidine | |
| 422 | 2-amino-4-[4-(4-chlorophenoxyacetyl)piperazin-1-yl]-6-(2- | 0.1 |
| | methoxyphenyl)pyrido-[3,2-d]pyrimidine | |
| 423 | 2-amino-4-[4-(4-chlorophenoxyacetyl)piperazin-1-yl]-6-(2,4- | 0.1 |
| | difluorophenyl)pyrido-[3,2-d]pyrimidine | |
| 424 | 2-amino-4-[4-(4-chlorophenoxyacetyl)piperazin-1-yl]-6-(2- | 0.11 |
| | fluorophenyl)pyrido-[3,2-d]pyrimidine | |
| 425 | 2-amino-4-[4-(4-chlorophenoxyacetyl)piperazin-1-yl]-6-(2,3- | 0.3 |
| | dichlorophenyl)-pyrido-[3,2-d]pyrimidine | |
| 426 | 2-amino-4-[4-(4-chlorophenoxyacetyl)piperazin-1-yl]-6-(4- | 0.03 |
| | methoxyphenyl)pyrido-[3,2-d]pyrimidine | |
| 427 | 2-amino-4-[4-(4-chlorophenoxyacetyl)piperazin-1-yl]-6-(2,4- | 0.3 |
| | dichlorophenyl)-pyrido-[3,2-d]pyrimidine | |
| 428 | 2-amino-4-[4-(4-chlorophenoxyacetyl)piperazin-1-yl]-6-(2,6- | 0.3 |
| | difluorophenyl)pyrido-[3,2-d]pyrimidine | |
| 429 | 2-amino-4-[4-(4-chlorophenoxyacetyl)piperazin-1-yl]-6-(2,5- | 0.5 |
| | dichlorophenyl)-pyrido-[3,2-d]pyrimidine | |
| 430 | 2-amino-4-[4-(4-chlorophenoxyacetyl)piperazin-1-yl]-6-(2- | 0.04 |
| | chlorophenyl)pyrido-[3,2-d]pyrimidine | |
| 431 | 2-amino-4-[4-(4-chlorophenoxyacetyl)piperazin-1-yl]-6-(5- | 0.3 |
| | chloro-2-fluorophenyl)-pyrido-[3,2-d]pyrimidine | |
| 432 | 2-amino-4-[4-(4-chlorophenoxyacetyl)piperazin-1-yl]-6- | 0.09 |
| | (3,4,5-trifluorophenyl)-pyrido-[3,2-d]pyrimidine | |
| 433 | 2-amino-4-[4-(4-chlorophenoxyacetyl)piperazin-1-yl]-6-(2,6- | 3.6 |
| | dimethylphenyl)-pyrido-[3,2-d]pyrimidine | |
| 434 | 2-amino-4-{4-[N-(tert-butoxycarbonyl)-glycyl]-piperazin-1-yl}- | 0.093 |
| | 6-(4-fluorophenyl)-pyrido[3,2-d]pyrimidine | |
| 435 | 2-amino-4-[4-(4-chlorophenoxyacetyl)piperazin-1-yl]-6-[N-3- | 0.006 |
| | (methanesulfonamidophenyl)]-pyrido-[3,2-d]pyrimidine | |
| 437 | 2-amino-4-[4-(2-hydroxyacetyl)piperazin-1-yl]-6-(4- | 1.6 |
| | fluorophenyl)-pyrido[3,2-d]pyrimidine | |
| 438 | 2-amino-4-[4-(4-chlorophenoxyacetyl)piperazin-1-yl]-6-(2,6- | 0.7 |
| | dichlorophenyl)-pyrido-[3,2-d]pyrimidine | |
| 439 | 2-amino-4-[4-(4-chlorophenoxyacetyl)piperazin-1-yl]-6-(4- | 0.3 |
| | trifluoromethoxyphenyl)-pyrido-[3,2-d]pyrimidine | |
| 440 | 2-amino-4-[4-(4-chlorophenoxyacetyl)piperazin-1-yl]-6-(2,5- | 0.07 |
| | difluorophenyl)pyrido-[3,2-d]pyrimidine | |
| 441 | 2-amino-4-[4-(4-chlorophenoxyacetyl)piperazin-1-yl]-6-[4- | 0.008 |
| | (hydroxymethyl)phenyl]pyrido-[3,2-d]pyrimidine | |
| 442 | 2-amino-4-[4-(4-chlorophenoxyacetyl)piperazin-1-yl]-6-(2- | 0.3 |
| | chloro-6-fluorophenyl)-pyrido-[3,2-d]pyrimidine | |
| 443 | 2-amino-4-[4-(4-chlorophenoxyacetyl)piperazin-1-yl]-6-[4- | 0.007 |
| | (methylaminocarbonyl)phenyl]-pyrido-[3,2-d]pyrimidine | |
| 444 | 2-amino-4-[4-(aminoacetyl)-piperazin-1-yl]-6-(4- | 2.3 |
| | fluorophenyl)-pyrido[3,2-d]pyrimidine | |
| 445 | 2-amino-4-[4-(4-chlorophenoxyacetyl)piperazin-1-yl]-6-(4- | 0.029 |
| | methylphenyl)pyrido-[3,2-d]pyrimidine | |
| 446 | 2-amino-4-[4-(4-chlorophenoxyacetyl)piperazin-1-yl]-6-(4- | 0.011 |
| | acetylphenyl)pyrido-[3,2-d]pyrimidine | |
| 447 | 2-amino-4-[4-(4-chlorophenoxyacetyl)piperazin-1-yl]-6-[4- | 0.4 |
| | (aminomethyl)phenyl]pyrido-[3,2-d]pyrimidine | |
| 448 | 2-amino-4-[4-(4-chlorophenoxyacetyl)piperazin-1-yl]-6-[4- | 0.015 |
| | (cyclopropylaminocarbonyl)phenyl]-pyrido-[3,2-d]pyrimidine | |
| 449 | 2-amino-4-[4-(4-chlorophenoxyacetyl)piperazin-1-yl]-6-[4- | 0.039 |
| | (acetamido)phenyl]-pyrido-[3,2-d]pyrimidine | |
| 450 | 2-amino-4-[4-(N-ethylcarbamoyl)-piperazin-1-yl]-6-(4- | 0.5 |
| | fluorophenyl)-pyrido[3,2-d]pyrimidine | |
| 451 | 2-amino-4-[4-(N-butylcarbamoyl)-piperazin-1-yl]-6-(4- | 0.2 |
| | fluorophenyl)-pyrido[3,2-d]pyrimidine | |
| 452 | 2-amino-4-[4-(N-methylcarbamoyl)-piperazin-1-yl]-6-(4- | 0.4 |
| | fluorophenyl)-pyrido[3,2-d]pyrimidine | |
| 453 | 2-amino-4-{4-[N-(1-adamantylcarbamoyl)]-piperazin-1-yl}-6- | 4 |
| | (4-fluorophenyl)-pyrido[3,2-d]pyrimidine | |
| 454 | 2-amino-4-[4-(N-cyclopentylcarbamoyl)-piperazin-1-yl]-6-(4- | 0.4 |
| | fluorophenyl)-pyrido[3,2-d]pyrimidine | |
| 455 | 2-amino-4-{4-[N-(4-chlorophenyl)carbamoyl]-piperazin-1-yl}- | 0.07 |
| | 6-(4-fluorophenyl)-pyrido[3,2-d]pyrimidine | |
| 457 | 2-amino-4-[1-(tert-butoxycarbonyl)piperid-3-ylamino]-6-(4- | 6.6 |
| | fluorophenyl)-pyrido[3,2-d]pyrimidine | |
| 458 | 2-amino-4-[4-(benzyloxycarbonyl)-piperazin-1-yl]-6-(4- | 0.7 |
| | fluorophenyl)-pyrido[3,2-d]pyrimidine | |
| 459 | 2-amino-4-{4-[2-(phenyl)ethylcarbamylmethyl]-piperazin-1- | 0.5 |
| | yl}-6-(4-fluorophenyl)-pyrido[3,2-d]pyrimidine | |
| 461 | 2-amino-4-[1-(tert-butoxycarbonyl)piperid-4-ylamino]-6-(4- | 4 |
| | fluorophenyl)-pyrido[3,2-d]pyrimidine | |
| 462 | 2-amino-4-[4-(methoxyacetyl)piperazin-1-yl]-6-(4- | 0.4 |
| | fluorophenyl)-pyrido[3,2-d]pyrimidine | |
| 463 | 2-amino-4-[4-(diethylcarbamyl)piperazin-1-yl]-6-(4- | 0.6 |
| | fluorophenyl)-pyrido[3,2-d]pyrimidine | |
| 464 | 2-amino-4-[4-(dimethylcarbamyl)piperazin-1-yl]-6-(4- | 0.8 |
| | fluorophenyl)-pyrido[3,2-d]pyrimidine | |
| 465 | 2-amino-4-[4-(diisopropylcarbamyl)piperazin-1-yl]-6-(4- | 3 |
| | fluorophenyl)-pyrido[3,2-d]pyrimidine | |
| 466 | 2-amino-4-[4-(morpholinocarbonyl)piperazin-1-yl]-6-(4- | 2.5 |
| | fluorophenyl)-pyrido[3,2-d]pyrimidine | |
| 467 | 2-amino-4-{4-[2-(4-chlorophenyl)-3-methylbutyryl]-piperazin- | 4.7 |
| | 1-yl}-6-(4-fluorophenyl)-pyrido[3,2-d]pyrimidine | |
| 468 | 2-amino-4-[4-(2-chloropropionyl)piperazin-1-yl]-6-(4- | 3.2 |
| | fluorophenyl)-pyrido[3,2-d]pyrimidine | |
| 469 | 2-amino-4-[4-(4-chlorophenoxycarbonyl)piperazin-1-yl]-6-(4- | 0.5 |
| | fluorophenyl)-pyrido[3,2-d]pyrimidine | |
| 472 | 2-amino-4-{4-[2-(4-chlorophenoxy)-2-methylpropionyl]- | >10 |
| | piperazin-1-yl}-6-(4-fluorophenyl)-pyrido[3,2-d]pyrimidine | |
| 473 | 2-amino-4-{4-[3-(4-chlorophenoxy)propionyl]-piperazin-1-yl}- | 0.5 |
| | 6-(4-fluorophenyl)-pyrido[3,2-d]pyrimidine | |
| 474 | 2-amino-4-[4-(2-phenoxypropionyl)piperazin-1-yl]-6-(4- | 1 |
| | fluorophenyl)-pyrido[3,2-d]pyrimidine | |
| 475 | {4-[2-amino-6-(4-fluorophenyl)-pyrido[3,2-d]pyrimidin-4-yl]- | 4.2 |
| | piperazin-1-yl}-acetic acid 4-chloro-benzyl ester | |
| 476 | N-(2-{4-[2-amino-6-(4-fluorophenyl)-pyrido[3,2-d]pyrimidin-4- | 0.06 |
| | yl]-piperazin-1-yl}-2-oxoethyl)-4-chlorobenzamide | |
| 477 | (S)-[2-{4-[2-amino-6-(4-fluorophenyl)-pyrido[3,2-d]pyrimidin- | 4.9 |
| | 4-yl]-piperazin-1-yl}-1-(4-chlorobenzyl)-2-oxoethyl]-carbamic | |
| | acid tert-butyl ester | |
| 478 | N-(2-{4-[2-amino-6-(4-fluorophenyl)-pyrido[3,2-d]pyrimidin-4- | 0.005 |
| | yl]-piperazin-1-yl}-2-oxoethyl)benzamide | |
| 480 | 2-amino-4-[1-(4-chlorophenoxyacetyl)piperid-4-ylamino]-6- | 2.2 |
| | (4-fluorophenyl)-pyrido[3,2-d]pyrimidine | |
| 481 | (R)-4-[2-amino-6-(4-fluorophenyl)-pyrido[3,2-d]pyrimidin-4- | >10 |
| | yl]-3-methylpiperazine-1-carboxylic acid tert-butyl ester | |
| 482 | (R)-2-amino-4-[4-(4-chlorophenoxyacetyl)-2- | 0.5 |
| | methylpiperazin-1-yl]-6-(4-fluorophenyl)-pyrido[3,2- | |
| | d]pyrimidine | |
| 483 | (S,S)-5-[2-amino-6-(4-fluorophenyl)-pyrido[3,2-d]pyrimidin-4- | 6.1 |
| | yl]-2,5-diaza-bicyclo[2.2.1]heptane-2-carboxylic acid tert- | |
| | butyl ester | |
| 484 | (S,S)-1-{5-[2-amino-6-(4-fluorophenyl)-pyrido[3,2- | 2.6 |
| | d]pyrimidin-4-yl]-2,5-diaza-bicyclo[2.2.1]hept-2-yl}-2-(4- | |
| | chlorophenoxy)-ethanone | |
| 485 | 2-amino-4-[1-(phenoxyacetyl)piperid-4-ylamino]-6-(4- | 0.3 |
| | fluorophenyl)-pyrido[3,2-d]pyrimidine | |
| 486 | (S)-4-[2-amino-6-(4-fluorophenyl)-pyrido[3,2-d]pyrimidin-4- | 0.8 |
| | yl]-3-methylpiperazine-1-carboxylic acid benzyl ester | |
| 487 | (R)-2-amino-4-(4-benzoyl-2-methylpiperazin-1-yl)-6-(4- | 7.5 |
| | fluorophenyl)-pyrido[3,2-d]pyrimidine | |
| 488 | (S)-2-amino-4-{4-[3-(4-chlorophenyl)-2- | 4.3 |
| | aminopropionyl]piperazin-1-yl}-6-(4-fluorophenyl)-pyrido[3,2- | |
| | d]pyrimidine | |
| 489 | (S)-2-amino-4-[4-(4-chlorophenoxyacetyl)-2- | 0.005 |
| | methylpiperazin-1-yl]-6-(4-fluorophenyl)-pyrido[3,2- | |
| | d]pyrimidine | |
| 490 | 2-amino-4-[4-(4-chlorophenylcarbamoyl-acetyl)piperazin-1- | 0.34 |
| | yl]-6-(4-fluorophenyl)-pyrido[3,2-d]pyrimidine | |
| 492 | 2-amino-4-(N-(4-chlorophenoxyacetyl)-piperazin-1-yl)-6- | 0.08 |
| | chloro-pyrido[3,2-d]pyrimidine | |
| 493 | 2-amino-4-[N-(4-chlorophenoxyacetyl)-piperazin-1-yl]-6-(3- | 0.057 |
| | chloro-4-ethoxyphenyl)-pyrido(3,2-d)pyrimidine | |
| 494 | 2-amino-4-[N-(4-chlorophenoxyacetyl)-piperazin-1-yl]-6-(4- | 0.033 |
| | ethoxyphenyl)-pyrido(3,2-d)pyrimidine | |
| 495 | 2-amino-4-[N-(4-chlorophenoxyacetyl)-piperazin-1-yl]-6-(4- | 0.022 |
| | methylthiophenyl)-pyrido(3,2-d)pyrimidine | |
| 496 | 2-amino-4-[N-(4-chlorophenoxyacetyl)-piperazin-1-yl]-6-(3- | 0.071 |
| | methoxy-4-hydroxyphenyl)-pyrido(3,2-d)pyrimidine | |
| 497 | 2-amino-4-[N-(4-chlorophenoxyacetyl)-piperazin-1-yl]-6-(2,3- | 0.21 |
| | dihydro-1-benzofuran-5-yl)-pyrido(3,2-d)pyrimidine | |
| 498 | 2-amino-4-[N-(4-chlorophenoxyacetyl)-piperazin-1-yl]-6-(3- | 0.008 |
| | methylphenyl)-pyrido(3,2-d)pyrimidine | |
| 499 | 2-amino-4-[N-(4-chlorophenoxyacetyl)-piperazin-1-yl]-6-(4- | 0.011 |
| | cyanomethylphenyl)-pyrido(3,2-d)pyrimidine | |
| 500 | 2-amino-4-[N-(4-chlorophenoxyacetyl)-piperazin-1-yl]-6-(3- | 0.031 |
| | methoxymethylphenyl)-pyrido(3,2-d)pyrimidine | |
| 501 | 2-amino-4-[N-(4-chlorophenoxyacetyl)-piperazin-1-yl]-6-(3- | >10 |
| | methoxy-4-benzyloxyphenyl)-pyrido(3,2-d)pyrimidine | |
| 502 | 2-amino-4-[N-(4-chlorophenoxyacetyl)-piperazin-1-yl]-6-(3,5- | 0.078 |
| | dimethyl-4-methoxyphenyl)-pyrido(3,2-d)pyrimidine | |
| 503 | 2-amino-4-[N-(4-chlorophenoxyacetyl)-piperazin-1-yl]-6-(4- | 0.02 |
| | cyanomethoxyphenyl)-pyrido(3,2-d)pyrimidine | |
| 504 | 2-amino-4-[N-(4-chlorophenoxyacetyl)-piperazin-1-yl]-6-(4- | 0.05 |
| | acetoxyphenyl)-pyrido(3,2-d)pyrimidine | |
| 505 | 2-amino-4-[N-(4-chlorophenoxyacetyl)-piperazin-1-yl]-6-(2,4- | 0.072 |
| | dimethoxyphenyl)-pyrido(3,2-d)pyrimidine | |
| 506 | 2-amino-4-[N-(4-chlorophenoxyacetyl)-piperazin-1-yl]-6-(2,5- | 0.69 |
| | dimethoxyphenyl)-pyrido(3,2-d)pyrimidine | |
| 507 | 2-amino-4-[N-(4-chlorophenoxyacetyl)-piperazin-1-yl]-6-(3- | 0.029 |
| | amino-4-methylphenyl)-pyrido(3,2-d)pyrimidine | |
| 508 | Synthesis of 2-amino-4-[N-(4-chlorophenoxyacetyl)- | 0.012 |
| | piperazin-1-yl]-6-(4-ethylphenyl)-pyrido(3,2-d)pyrimidine | |
| 509 | 2-amino-4-[N-(4-chlorophenoxyacetyl)-piperazin-1-yl]-6-(3- | 0.031 |
| | methyl-4-methoxyphenyl)-pyrido(3,2-d)pyrimidine | |
| 510 | 2-amino-4-[N-(4-chlorophenoxyacetyl)-piperazin-1-yl]-6-(4- | 0.032 |
| | ethoxycarbonylphenyl)-pyrido(3,2-d)pyrimidine | |
| 511 | 2-amino-4-[N-(4-chlorophenoxyacetyl)-piperazin-1-yl]-6-(4- | 0.054 |
| | methyl-2-thienyl)-pyrido(3,2-d)pyrimidine | |
| 512 | 2-amino-4-[N-(4-chlorophenoxyacetyl)-piperazin-1-yl]-6-(2- | 0.008 |
| | methyl-phenyl)-pyrido(3,2-d)pyrimidine | |
| 513 | 2-amino-4-[N-(4-chlorophenoxyacetyl)-piperazin-1-yl]-6-(3- | >10 |
| | fluoro-4-benzyloxy-phenyl)-pyrido(3,2-d)pyrimidine | |
| 514 | 2-amino-4-[N-(4-chlorophenoxyacetyl)-piperazin-1-yl]-6-(3- | 0.071 |
| | methoxy-4-amino-phenyl)-pyrido(3,2-d)pyrimidine | |
| 516 | 2-amino-4-[N-(4-chlorophenoxyacetyl)-piperazin-1-yl]-6-(3,4- | 0.056 |
| | dimethyl-phenyl)-pyrido(3,2-d)pyrimidine | |
| 517 | 2-amino-4-[N-(4-chlorophenoxyacetyl)-piperazin-1-yl]-6-(3,4- | 0.012 |
| | dimethyl-phenyl)-pyrido(3,2-d)pyrimidine | |
| 524 | [4-(2-amino-6-(4-fluorophenyl)pyrido[3,2-d]pyrimidin-4- | 0.225 |
| | yl)piperazin-1-yl]chroman-2-ylmethanone | |
| 531 | 2-methyl-4-[N-(3-methylphenylcarbamoyl)piperazin-1-yl]-6- | 0.084 |
| | (4-fluorophenyl)-pyrido[3,2-d]pyrimidine | |
| 533 | 2-phenyl-4-[N-(3-methylphenylcarbamoyl)piperazin-1-yl]-6- | 2.6 |
| | (4-fluorophenyl)-pyrido[3,2-d]pyrimidine | |
| 537 | 2-(N,N-dimethylamino)methyl-4-[N-(3- | 3.3 |
| | methylphenylcarbamoyl)piperazin-1-yl]-6-(4-fluorophenyl)- | |
| | pyrido[3,2-d]pyrimidine | |
| 538 | 2-(N-methoxyethylamino)methyl-4-[N-(3- | 0.34 |
| | methylphenylcarbamoyl)piperazin-1-yl]-6-(4-fluorophenyl)- | |
| | pyrido[3,2-d]pyrimidine | |
| 539 | 2-(N-cyclopropylamino)methyl-4-[N-(3- | 0.7 |
| | methylphenylcarbamoyl)piperazin-1-yl]-6-(4-fluorophenyl)- | |
| | pyrido[3,2-d]pyrimidine | |
| 541 | 2-amino-4-[(3-methylphenyl carbamoyl)-piperazin-1-yl]-6- | 0.06 |
| | chloro-pyrido[3,2-d]pyrimidine | |
| 542 | 2-amino-4-[(3-methylphenyl carbamoyl)-piperazin-1-yl]-6-(4- | 0.005 |
| | pyridyl)-pyrido[3,2-d]pyrimidine | |
| 543 | 2-amino-4-[(3-methylphenyl carbamoyl)-piperazin-1-yl]-6-(4- | 0.03 |
| | cyanophenyl)-pyrido[3,2-d]pyrimidine | |
| 545 | 2-amino-4-[(3-methylphenyl carbamoyl)-piperazin-1-yl]-6-(3- | 0.03 |
| | cyanophenyl)-pyrido[3,2-d]pyrimidine | |
| 546 | 2-amino-4-[(3-methylphenyl carbamoyl)-piperazin-1-yl]-6-(2- | 0.004 |
| | cyanophenyl)-pyrido[3,2-d]pyrimidine | |
| 547 | 2-amino-4-[(3-methylphenyl carbamoyl)-piperazin-1-yl]-6-(2- | 0.06 |
| | fluorophenyl)-pyrido[3,2-d]pyrimidine | |
| 548 | 2-amino-4-[(3-methylphenyl carbamoyl)-piperazin-1-yl]-6-(4- | 0.17 |
| | trifluoromethylphenyl)-pyrido[3,2-d]pyrimidine | |
| 549 | 2-amino-4-[(3-methylphenyl carbamoyl)-piperazin-1-yl]-6-(2- | 0.08 |
| | chlorophenyl)-pyrido[3,2-d]pyrimidine | |
| 550 | 2-amino-4-[(3-methylphenyl carbamoyl)-piperazin-1-yl]-6- | 3.5 |
| | (2,6-dimethylphenyl)-pyrido[3,2-d]pyrimidine | |
| 551 | 2-amino-4-[(3-methylphenyl carbamoyl)-piperazin-1-yl]-6- | 0.5 |
| | (2,4,6-trifluorophenyl)-pyrido[3,2-d]pyrimidine | |
| 553 | 4-[(3-methylphenyl carbamoyl)-piperazin-1-yl]-6-(4- | 0.06 |
| | cyanophenyl)pyrido[3,2-d]pyrimidine | |
| 554 | 4-[(3-methylphenyl carbamoyl)-piperazin-1-yl]-6-(3- | 0.037 |
| | cyanophenyl)pyrido[3,2-d]pyrimidine | |
| 555 | 4-[(3-methylphenyl carbamoyl)-piperazin-1-yl]-6-(3-methyl-4- | 0.3 |
| | fluorophenyl)pyrido[3,2-d]pyrimidine | |
| 556 | 4-[(3-methylphenyl carbamoyl)-piperazin-1-yl]-6-(2,4- | 0.1 |
| | difluorophenyl)pyrido[3,2-d]pyrimidine | |
| 557 | 4-[(3-methylphenyl carbamoyl)-piperazin-1-yl]-6-(2- | 0.03 |
| | cyanophenyl)pyrido[3,2-d]pyrimidine | |
| 558 | 4-[(3-methylphenyl carbamoyl)-piperazin-1-yl]-6-(4- | 0.039 |
| | fluorophenyl)pyrido[3,2-d]pyrimidine | |
| 559 | 4-[(3-methylphenyl carbamoyl)-piperazin-1-yl]-6-(3-fluoro-4- | 0.178 |
| | ethoxyphenyl)pyrido[3,2-d]pyrimidine | |
| 560 | 4-[(3-methylphenyl carbamoyl)-piperazin-1-yl]-6-(3-chloro-4- | 0.38 |
| | ethoxyphenyl)pyrido[3,2-d]pyrimidine | |
| 561 | 4-[(3-methylphenyl carbamoyl)-piperazin-1-yl]-6-(3,4,5- | 0.42 |
| | trifluorophenyl)pyrido[3,2-d]pyrimidine | |
| 562 | 4-[(3-methylphenyl carbamoyl)-piperazin-1-yl]-6-(2- | 0.053 |
| | thienyl)pyrido[3,2-d]pyrimidine | |
| 563 | 4-[(3-methylphenyl carbamoyl)-piperazin-1-yl]-6-(2- | 0.036 |
| | furanyl)pyrido[3,2-d]pyrimidine | |
| 566 | 2-chloro-6-(4-fluorophenyl)-4-(4-Boc-piperazino)pyrido[3,2- | >10 |
| | d]pyrimidine | |
| 567 | 6-(4-fluorophenyl)-4-(4-Boc-piperazino)-2-pyrrolidino- | >10 |
| | pyrido[3,2-d]pyrimidine | |
| 568 | 2-cyclopentylamino-6-(4-fluorophenyl)-4-(4-Boc-piperazino)- | 6.5 |
| | pyrido[3,2-d]pyrimidine | |
| 569 | 2,4-dimethylamino-6-(4-fluorophenyl)pyrido[3,2-d]pyrimidine | 5.9 |
| 571 | 2-cyclopentylamino-6-(4-fluorophenyl)-4-[(3-methylphenyl | 0.5 |
| | carbamoyl)-piperazin-1-yl]--pyrido[3,2-d]pyrimidine | |
| 573 | 2-cyclopentylamino-6-(4-fluorophenyl)-4-[N-(4- | 8 |
| | chlorophenoxyacetyl)-piperazin-1-yl]-pyrido[3,2-d]pyrimidine | |
| 574 | 2-methylamino-4-[N-(4-chlorophenoxyacetyl)-piperazin-1-yl]- | 0.36 |
| | 6-(4-fluorophenyl)-pyrido[3,2-d]pyrimidine | |
| 575 | 2-amino-4-[(R)-3-Boc-amino-pyrrolidin-1-yl]-6-(4- | >10 |
| | fluorophenyl)-pyrido(3,2-d)pyrimidine | |
| 576 | 2-amino-4-[(S)-3-Boc-amino-pyrrolidin-1-yl]-6-(4- | 4.2 |
| | fluorophenyl)-pyrido(3,2-d)pyrimidine | |
| 577 | 2-amino-4-[(S)-3-Boc-amino-pyrrolidin-1-yl]-6-(4- | 4.4 |
| | fluorophenyl)-pyrido(3,2-d)pyrimidine | |
| 578 | 2-amino-4-[3-(S)-3-methyl phenyl carbamoyl pyrrolidin-1-yl]- | >10 |
| | pyrido(3,2-d)pyrimidine | |
| 579 | 2-amino-4-[3-(R)-3-methyl phenyl carbamoyl pyrrolidin-1-yl]- | >10 |
| | pyrido(3,2-d)pyrimidine | |
| 580 | 2-amino-4-[(S)-3-methyl-phenyl carbamoyl amino-pyrrolidin- | 0.3 |
| | 1-yl]-6-(4-fluorophenyl)-pyrido(3,2-d)pyrimidine | |

### EXAMPLES 584 TO 628

### Synthesis of 2-amino-4-[4-(N-butylcarbamoyl)-piperazin-1-yl-6-(3,4-dimethoxyphenyl)-pyrido[3,2-d]pyrimidine analogues

The synthetic procedure of example 397 is repeated, except that n-butyl isocyanate is replaced with the same molar amount of one of the following reactants: methyl isocyanate, ethyl isocyanate, n-propyl isocyanate, n-pentyl isocyanate, n-octyl isocyanate, cyclopentyl isocyanate, cyclohexyl isocyanate, adamantyl isocyanate, benzyl isocyanate, D-α-methylbenzyl isocyanate, L-α-methylbenzyl isocyanate, 2-methylbenzyl isocyanate, 3-methylbenzyl isocyanate, 4-methylbenzyl isocyanate, 2-methoxybenzyl isocyanate, 3-methoxybenzyl isocyanate, 4-methoxybenzyl isocyanate, 2-fluorobenzyl isocyanate, 3-fluorobenzyl isocyanate, 4-fluorobenzyl isocyanate, 2-chlorobenzyl isocyanate, 3-chlorobenzyl isocyanate, 4-chlorobenzyl isocyanate, methyl isothiocyanate, ethyl isothiocyanate, n-propyl isothiocyanate, n-butyl isothiocyanate, n-pentyl isothiocyanate, n-hexyl isothiocyanate, n-octyl isothiocyanate, cyclopentyl isothiocyanate, cyclohexyl isothiocyanate, benzyl isothiocyanate, D-α-methylbenzyl isothiocyanate, L-α-methylbenzyl isothiocyanate, 2-methylbenzyl isothiocyanate, 3-methylbenzyl isothiocyanate, 4-methylbenzyl isothiocyanate, 4-methoxybenzyl isothiocyanate, 2-fluorobenzyl isothiocyanate, 3-fluorobenzyl isothiocyanate, 4-fluorobenzyl isothiocyanate, 2-chlorobenzyl isothiocyanate, 3-chlorobenzyl isothiocyanate and 4-chlorobenzyl isothiocyanate.

In this way the following compounds of the invention are obtained in high yields similar to example 397:
2-amino-4-[4-(N-methylcarbamoyl)-piperazin-1-yl]-6-(3,4-dimethoxy-phenyl)-pyrido[3,2-d]pyrimidine (example 584),
2-amino-4-[4-(N-ethylcarbamoyl)-piperazin-1-yl]-6-(3,4-dimethoxyphenyl)-pyrido[3,2-d]pyrimidine (example 585),
2-amino-4-[4-(N-propylcarbamoyl)-piperazin-1-yl]-6-(3,4-dimethoxy-phenyl)-pyrido[3,2-d]pyrimidine (example 586),
2-amino-4-[4-(N-pentylcarbamoyl)-piperazin-1-yl]-6-(3,4-dimethoxy-phenyl)-pyrido[3,2-d]pyrimidine (example 587),
2-amino-4-[4-(N-octylcarbamoyl)-piperazin-1-yl]-6-(3,4-dimethoxy-phenyl)-pyrido[3,2-d]pyrimidine (example 588),
2-amino-4-[4-(N-cyclopentylcarbamoyl)-piperazin-1-yl]-6-(3,4-dimethoxy-phenyl)-pyrido[3,2-d]pyrimidine (example 589),
2-amino-4-[4-(N-cyclohexylcarbamoyl)-piperazin-1-yl]-6-(3,4-dimethoxy-phenyl)-pyrido[3,2-d]pyrimidine (example 590),
2-amino-4-[4-(N-adamantylcarbamoyl)-piperazin-1-yl]-6-(3,4-dimethoxy-phenyl)-pyrido[3,2-d]pyrimidine (example 591),
2-amino-4-[4-(N-benzylcarbamoyl)-piperazin-1-yl]-6-(3,4-dimethoxy-phenyl)-pyrido[3,2-d]pyrimidine (example 592),
2-amino-4-[4-(N-D-α-methylbenzylcarbamoyl)-piperazin-1-yl]-6-(3,4-dimethoxy-phenyl)-pyrido[3,2-d]pyrimidine (example 593),
2-amino-4-[4-(N-L-α-methylbenzylcarbamoyl)-piperazin-1-yl]-6-(3,4-dimethoxy-phenyl)-pyrido[3,2-d]pyrimidine (example 594),
2-amino-4-[4-(N-2-methylbenzylcarbamoyl)-piperazin-1-yl]-6-(3,4-dimethoxy-phenyl)-pyrido[3,2-d]pyrimidine (example 595),
2-amino-4-[4-(N-3-methylbenzylcarbamoyl)-piperazin-1-yl]-6-(3,4-dimethoxy-phenyl)-pyrido[3,2-d]pyrimidine (example 596),
2-amino-4-[4-(N-4-methylbenzylcarbamoyl)-piperazin-1-yl]-6-(3,4-dimethoxy-phenyl)-pyrido[3,2-d]pyrimidine (example 597),
2-amino-4-[4-(N-2-methoxybenzylcarbamoyl)-piperazin-1-yl]-6-(3,4-dimethoxy-phenyl)-pyrido[3,2-d]pyrimidine (example 598),
2-amino-4-[4-(N-3-methoxybenzylcarbamoyl)-piperazin-1-yl]-6-(3,4-dimethoxy-phenyl)-pyrido[3,2-d]pyrimidine (example 599),
2-amino-4-[4-(N-4-methoxybenzylcarbamoyl)-piperazin-1-yl]-6-(3,4-dimethoxy-phenyl)-pyrido[3,2-d]pyrimidine (example 600),
2-amino-4-[4-(N-2-fluorobenzylcarbamoyl)-piperazin-1-yl]-6-(3,4-dimethoxy-phenyl)-pyrido[3,2-d]pyrimidine (example 601),
2-amino-4-[4-(N-3-fluorobenzylcarbamoyl)-piperazin-1-yl]-6-(3,4-dimethoxy-phenyl)-pyrido[3,2-d]pyrimidine (example 602),
2-amino-4-[4-(N-4-fluorobenzylcarbamoyl)-piperazin-1-yl]-6-(3,4-dimethoxy-phenyl)-pyrido[3,2-d]pyrimidine (example 603),
2-amino-4-[4-(N-2-chlorobenzylcarbamoyl)-piperazin-1-yl]-6-(3,4-dimethoxy-phenyl)-pyrido[3,2-d]pyrimidine (example 604),
2-amino-4-[4-(N-3-chlorobenzylcarbamoyl)-piperazin-1-yl]-6-(3,4-dimethoxy-phenyl)-pyrido[3,2-d]pyrimidine (example 605),
2-amino-4-[4-(N-4-chlorobenzylcarbamoyl)-piperazin-1-yl]-6-(3,4-dimethoxy-phenyl)-pyrido[3,2-d]pyrimidine (example 606),
2-amino-4-[4-(N-methylthiocarbamoyl)-piperazin-1-yl]-6-(3,4-dimethoxy-phenyl)-pyrido[3,2-d]pyrimidine (example 607),
2-amino-4-[4-(N-ethylthiocarbamoyl)-piperazin-1-yl]-6-(3,4-dimethoxyphenyl)-pyrido[3,2-d]pyrimidine (example 608),
2-amino-4-[4-(N-propylthiocarbamoyl)-piperazin-1-yl]-6-(3,4-dimethoxy-phenyl)-pyrido[3,2-d]pyrimidine (example 609),
2-amino-4-[4-(N-butylthiocarbamoyl)-piperazin-1-yl]-6-(3,4-dimethoxy-phenyl)-pyrido[3,2-d]pyrimidine (example 610),
2-amino-4-[4-(N-pentylthiocarbamoyl)-piperazin-1-yl]-6-(3,4-dimethoxy-phenyl)-pyrido[3,2-d]pyrimidine (example 611),
2-amino-4-[4-(N-hexylthiocarbamoyl)-piperazin-1-yl]-6-(3,4-dimethoxy-phenyl)-pyrido[3,2-d]pyrimidine (example 612),
2-amino-4-[4-(N-octylthiocarbamoyl)-piperazin-1-yl]-6-(3,4-dimethoxy-phenyl)-pyrido[3,2-d]pyrimidine (example 613),
2-amino-4-[4-(N-cyclopentylthiocarbamoyl)-piperazin-1-yl]-6-(3,4-dimethoxy-phenyl)-pyrido[3,2-d]pyrimidine (example 614),
2-amino-4-[4-(N-cyclohexylthiocarbamoyl)-piperazin-1-yl]-6-(3,4-dimethoxy-phenyl)-pyrido[3,2-d]pyrimidine (example 615),
2-amino-4-[4-(N-benzylthiocarbamoyl)-piperazin-1-yl]-6-(3,4-dimethoxy-phenyl)-pyrido[3,2-d]pyrimidine (example 616),
2-amino-4-[4-(N-D-α-methylbenzylthiocarbamoyl)-piperazin-1-yl]-6-(3,4-dimethoxy-phenyl)-pyrido[3,2-d]pyrimidine (example 617),
2-amino-4-[4-(N-L-α-methylbenzylthiocarbamoyl)-piperazin-1-yl]-6-(3,4-dimethoxy-phenyl)-pyrido[3,2-d]pyrimidine (example 618),
2-amino-4-[4-(N-2-methylbenzylthiocarbamoyl)-piperazin-1-yl]-6-(3,4-dimethoxy-phenyl)-pyrido[3,2-d]pyrimidine (example 619),
2-amino-4-[4-(N-3-methylbenzylthiocarbamoyl)-piperazin-1-yl]-6-(3,4-dimethoxy-phenyl)-pyrido[3,2-d]pyrimidine (example 620),
2-amino-4-[4-(N-4-methylbenzylthiocarbamoyl)-piperazin-1-yl]-6-(3,4-dimethoxy-phenyl)-pyrido[3,2-d]pyrimidine (example 621),
2-amino-4-[4-(N-4-methoxybenzylthiocarbamoyl)-piperazin-1-yl]-6-(3,4-dimethoxy-phenyl)-pyrido[3,2-d]pyrimidine (example 622),
2-amino-4-[4-(N-2-fluorobenzylthiocarbamoyl)-piperazin-1-yl]-6-(3,4-dimethoxy-phenyl)-pyrido[3,2-d]pyrimidine (example 623),
2-amino-4-[4-(N-3-fluorobenzylthiocarbamoyl)-piperazin-1-yl]-6-(3,4-dimethoxy-phenyl)-pyrido[3,2-d]pyrimidine (example 624),
2-amino-4-[4-(N-4-fluorobenzylthiocarbamoyl)-piperazin-1-yl]-6-(3,4-dimethoxy-phenyl)-pyrido[3,2-d]pyrimidine (example 625),
2-amino-4-[4-(N-2-chlorobenzylthiocarbamoyl)-piperazin-1-yl]-6-(3,4-dimethoxy-phenyl)-pyrido[3,2-d]pyrimidine (example 626),
2-amino-4-[4-(N-3-chlorobenzylthiocarbamoyl)-piperazin-1-yl]-6-(3,4-dimethoxy-phenyl)-pyrido[3,2-d]pyrimidine (example 627), and
2-amino-4-[4-(N-4-chlorobenzylthiocarbamoyl)-piperazin-1-yl]-6-(3,4-dimethoxy-phenyl)-pyrido[3,2-d]pyrimidine (example 628).

### EXAMPLES 629 TO 670

### Synthesis of 2-amino-4-[4-(N-ethylcarbamoyl)-piperazin-1-yl]-6-(4-fluorophenyl)-pyrido[3,2-d]pyrimidine analogues

The synthetic procedure of example 450 is repeated, except that ethyl isocyanate is replaced with the same molar amount of one of the following reactants: n-propyl isocyanate, n-pentyl isocyanate, n-hexyl isocyanate, n-octyl isocyanate, cyclohexyl isocyanate, benzyl isocyanate, D-α-methylbenzyl isocyanate, L-α-methylbenzyl isocyanate, 2-methylbenzyl isocyanate, 3-methylbenzyl isocyanate, 4-methylbenzyl isocyanate, 2-methoxybenzyl isocyanate, 3-methoxybenzyl isocyanate, 4-methoxybenzyl isocyanate, 2-fluorobenzyl isocyanate, 3-fluorobenzyl isocyanate, 4-fluorobenzyl isocyanate, 2-chlorobenzyl isocyanate, 3-chlorobenzyl isocyanate, 4-chlorobenzyl isocyanate, methyl isothiocyanate, ethyl isothiocyanate, n-propyl isothiocyanate, n-butyl isothiocyanate, n-pentyl isothiocyanate, n-hexyl isothiocyanate, n-octyl isothiocyanate, cyclopentyl isothiocyanate, cyclohexyl isothiocyanate, benzyl isothiocyanate, D-α-methylbenzyl isothiocyanate, L-α-methylbenzyl isothiocyanate, 2-methylbenzyl isothiocyanate, 3-methylbenzyl isothiocyanate, 4-methylbenzyl isothiocyanate, 4-methoxybenzyl isothiocyanate, 2-fluorobenzyl isothiocyanate, 3-fluorobenzyl isothiocyanate, 4-fluorobenzyl isothiocyanate, 2-chlorobenzyl isothiocyanate, 3-chlorobenzyl isothiocyanate and 4-chlorobenzyl isothiocyanate.

In this way the following compounds of the invention are obtained in yields similar to example 450:
2-amino-4-[4-(N-propylcarbamoyl)-piperazin-1-yl]-6-(4-fluorophenyl)-pyrido[3,2-d]pyrimidine (example 629),
2-amino-4-[4-(N-pentylcarbamoyl)-piperazin-1-yl]-6-(4-fluorophenyl)-pyrido[3,2-d]pyrimidine (example 630),
2-amino-4-[4-(N-hexylcarbamoyl)-piperazin-1-yl]-6-(4-fluorophenyl)-pyrido[3,2-d]pyrimidine (example 631),
2-amino-4-[4-(N-octylcarbamoyl)-piperazin-1-yl]-6-(4-fluorophenyl)-pyrido[3,2-d]pyrimidine (example 632),
2-amino-4-[4-(N-cyclohexylcarbamoyl)-piperazin-1-yl]-6-(4-fluorophenyl)-pyrido[3,2-d]pyrimidine (example 633),
2-amino-4-[4-(N-benzylcarbamoyl)-piperazin-1-yl]-6-(4-fluorophenyl)-pyrido[3,2-d]pyrimidine (example 634),
2-amino-4-[4-(N-D-α-methylbenzylcarbamoyl)-piperazin-1-yl]-6-(4-fluorophenyl)-pyrido[3,2-d]pyrimidine (example 635),
2-amino-4-[4-(N-L-α-methylbenzylcarbamoyl)-piperazin-1-yl]-6-(4-fluorophenyl)-pyrido[3,2-d]pyrimidine (example 636),
2-amino-4-[4-(N-2-methylbenzylcarbamoyl)-piperazin-1-yl]-6-(4-fluorophenyl)-pyrido[3,2-d]pyrimidine (example 637),
2-amino-4-[4-(N-3-methylbenzylcarbamoyl)-piperazin-1-yl]-6-(4-fluorophenyl)-pyrido[3,2-d]pyrimidine (example 638),
2-amino-4-[4-(N-4-methylbenzylcarbamoyl)-piperazin-1-yl]-6-(4-fluorophenyl)-pyrido[3,2-d[pyrimidine (example 639),
2-amino-4-[4-(N-2-methoxybenzylcarbamoyl)-piperazin-1-yl]-6-(4-fluorophenyl)-pyrido[3,2-d]pyrimidine (example 640),
2-amino-4-[4-(N-3-methoxybenzylcarbamoyl)-piperazin-1-yl]-6-(4-fluorophenyl)-pyrido[3,2-d]pyrimidine (example 641),
2-amino-4-[4-(N-4-methoxybenzylcarbamoyl)-piperazin-1-yl]-6-(4-fluorophenyl)-pyrido[3,2-d]pyrimidine (example 642),
2-amino-4-[4-(N-2-fluorobenzylcarbamoyl)-piperazin-1-yl]-6-(4-fluorophenyl)-pyrido[3,2-d]pyrimidine (example 643),
2-amino-4-[4-(N-3-fluorobenzylcarbamoyl)-piperazin-1-yl]-6-(4-fluorophenyl)-pyrido[3,2-d]pyrimidine (example 644),
2-amino-4-[4-(N-4-fluorobenzylcarbamoyl)-piperazin-1-yl]-6-(4-fluorophenyl)-pyrido[3,2-d]pyrimidine (example 645),
2-amino-4-[4-(N-2-chlorobenzylcarbamoyl)-piperazin-1-yl]-6-(4-fluorophenyl)-pyrido[3,2-d]pyrimidine (example 646),
2-amino-4-[4-(N-3-chlorobenzylcarbamoyl)-piperazin-1-yl]-6-(4-fluorophenyl)-pyrido[3,2-d]pyrimidine (example 647),
2-amino-4-[4-(N-4-chlorobenzylcarbamoyl)-piperazin-1-yl]-6-(4-fluorophenyl)-pyrido[3,2-d]pyrimidine (example 648),
2-amino-4-[4-(N-methylthiocarbamoyl)-piperazin-1-yl]-6-(4-fluorophenyl)-pyrido[3,2-d]pyrimidine (example 649),
2-amino-4-[4-(N-ethylthiocarbamoyl)-piperazin-1-yl]-6-(4-fluorophenyl)-pyrido[3,2-d]pyrimidine (example 650),
2-amino-4-[4-(N-propylthiocarbamoyl)-piperazin-1-yl]-6-(4-fluorophenyl)-pyrido[3,2-d]pyrimidine (example 651),
2-amino-4-[4-(N-butylthiocarbamoyl)-piperazin-1-yl]-6-(4-fluorophenyl)-pyrido[3,2-d]pyrimidine (example 652),
2-amino-4-[4-(N-pentylthiocarbamoyl)-piperazin-1-yl]-6-(4-fluorophenyl)-pyrido[3,2-d]pyrimidine (example 653),
2-amino-4-[4-(N-hexylthiocarbamoyl)-piperazin-1-yl]-6-(4-fluorophenyl)-pyrido[3,2-d]pyrimidine (example 654),
2-amino-4-[4-(N-octylthiocarbamoyl)-piperazin-1-yl]-6-(4-fluorophenyl)-pyrido[3,2-d]pyrimidine (example 655),
2-amino-4-[4-(N-cyclopentylthiocarbamoyl)-piperazin-1-yl]-6-(4-fluorophenyl)-pyrido[3,2-d]pyrimidine (example 656),
2-amino-4-[4-(N-cyclohexylthiocarbamoyl)-piperazin-1-yl]-6-(4-fluorophenyl)-pyrido[3,2-d]pyrimidine (example 657),
2-amino-4-[4-(N-benzylthiocarbamoyl)-piperazin-1-yl]-6-(4-fluorophenyl)-pyrido[3,2-d]pyrimidine (example 658),
2-amino-4-[4-(N-D-α-methylbenzylthiocarbamoyl)-piperazin-1-yl]-6-(4-fluorophenyl)-pyrido[3,2-d]pyrimidine (example 659),
2-amino-4-[4-(N-L-α-methylbenzylthiocarbamoyl)-piperazin-1-yl]-6-(4-fluorophenyl)-pyrido[3,2-d]pyrimidine (example 660),
2-amino-4-[4-(N-2-methylbenzylthiocarbamoyl)-piperazin-1-yl]-6-(4-fluorophenyl)-pyrido[3,2-d]pyrimidine (example 661),
2-amino-4-[4-(N-3-methylbenzylthiocarbamoyl)-piperazin-1-yl]-6-(4-fluorophenyl)-pyrido[3,2-d]pyrimidine (example 662),
2-amino-4-[4-(N-4-methylbenzylthiocarbamoyl)-piperazin-1-yl]-6-(4-fluorophenyl)-pyrido[3,2-d]pyrimidine (example 663),
2-amino-4-[4-(N-4-methoxybenzylthiocarbamoyl)-piperazin-1-yl]-6-(4-fluorophenyl)-pyrido[3,2-d]pyrimidine (example 664),
2-amino-4-[4-(N-2-fluorobenzylthiocarbamoyl)-piperazin-1-yl]-6-(4-fluorophenyl)-pyrido[3,2-d]pyrimidine (example 665),
2-amino-4-[4-(N-3-fluorobenzylthiocarbamoyl)-piperazin-1-yl]-6-(4-fluorophenyl)-pyrido[3,2-d]pyrimidine (example 666),
2-amino-4-[4-(N-4-fluorobenzylthiocarbamoyl)-piperazin-1-yl]-6-(4-fluorophenyl)-pyrido[3,2-d]pyrimidine (example 667),
2-amino-4-[4-(N-2-chlorobenzylthiocarbamoyl)-piperazin-1-yl]-6-(4-fluorophenyl)-pyrido[3,2-d]pyrimidine (example 668),
2-amino-4-[4-(N-3-chlorobenzylthiocarbamoyl)-piperazin-1-yl]-6-(4-fluorophenyl)-pyrido[3,2-d]pyrimidine (example 669), and
2-amino-4-[4-(N-4-chlorobenzylthiocarbamoyl)-piperazin-1-yl]-6-(4-fluorophenyl)-pyrido[3,2-d]pyrimidine (example 670).

### EXAMPLES 671 TO 714

### Synthesis of 2-chloromethyl-4-[N-(3-methylbenzylcarbamoyl)-piperazin-1-yl]-6-(4-fluorophenyl)-pyrido[3,2-d]pyrimidine analogues

The synthetic procedure of example 536 is repeated, except that 3-methylbenzyl isocyanate is replaced with the same molar amount of one of the following reactants: methyl isocyanate, ethyl isocyanate, n-propyl isocyanate, n-butyl isocyanate, n-pentyl isocyanate, n-hexyl isocyanate, n-octyl isocyanate, cyclohexyl isocyanate, benzyl isocyanate, adamantyl isocyanate, D-α-methylbenzyl isocyanate, L-α-methylbenzyl isocyanate, 2-methylbenzyl isocyanate, 4-methylbenzyl isocyanate, 2-methoxybenzyl isocyanate, 3-methoxybenzyl isocyanate, 4-methoxybenzyl isocyanate, 2-fluorobenzyl isocyanate, 3-fluorobenzyl isocyanate, 4-fluorobenzyl isocyanate, 2-chlorobenzyl isocyanate, 3-chlorobenzyl isocyanate, 4-chlorobenzyl isocyanate, methyl isothiocyanate, ethyl isothiocyanate, n-propyl isothiocyanate, n-butyl isothiocyanate, n-pentyl isothiocyanate, n-hexyl isothiocyanate, n-octyl isothiocyanate, cyclopentyl isothiocyanate, cyclohexyl isothiocyanate, benzyl isothiocyanate, D-α-methylbenzyl isothiocyanate, L-α-methylbenzyl isothiocyanate, 2-methylbenzyl isothiocyanate, 3-methylbenzyl isothiocyanate, 4-methylbenzyl isothiocyanate, 4-methoxybenzyl isothiocyanate, 2-fluorobenzyl isothiocyanate, 3-fluorobenzyl isothiocyanate, 4-fluorobenzyl isothiocyanate, 2-chlorobenzyl isothiocyanate, 3-chlorobenzyl isothiocyanate and 4-chlorobenzyl isothiocyanate.

In this way the following compounds of the invention are obtained in high yields similar to example 536:
2-chloromethyl-4-[4-(N-methylcarbamoyl)-piperazin-1-yl]-6-(4-fluoro-phenyl)-pyrido[3,2-d]pyrimidine (example 671),
2-chloromethyl-4-[4-(N-ethylcarbamoyl)-piperazin-1-yl]-6-(4-fluorophenyl)-pyrido[3,2-d]pyrimidine (example 672),
2-chloromethyl-4-[4-(N-propylcarbamoyl)-piperazin-1-yl]-6-(4-fluorophenyl)-pyrido[3,2-d]pyrimidine (example 673),
2-chloromethyl-4-[4-(N-butylcarbamoyl)-piperazin-1-yl]-6-(4-fluorophenyl)-pyrido[3,2-d]pyrimidine (example 674),
2-chloromethyl-4-[4-(N-pentylcarbamoyl)-piperazin-1-yl]-6-(4-fluorophenyl)-pyrido[3,2-d]pyrimidine (example 675),
2-chloromethyl-4-[4-(N-hexylcarbamoyl)-piperazin-1-yl]-6-(4-fluorophenyl)-pyrido[3,2-d]pyrimidine (example 676),
2-chloromethyl-4-[4-(N-octylcarbamoyl)-piperazin-1-yl]-6-(4-fluorophenyl)-pyrido[3,2-d]pyrimidine (example 677),
2-chloromethyl-4-[4-(N-cyclohexylcarbamoyl)-piperazin-1-yl]-6-(4-fluorophenyl)-pyrido[3,2-d]pyrimidine (example 678),
2-chloromethyl-4-[4-(N-benzylcarbamoyl)-piperazin-1-yl]-6-(4-fluorophenyl)-pyrido[3,2-d]pyrimidine (example 679),
2-chloromethyl-4-[4-(N-D-α-methylbenzylcarbamoyl)-piperazin-1-yl]-6-(4-fluorophenyl)-pyrido[3,2-d]pyrimidine (example 680),
2-chloromethyl-4-[4-(N-L-α-methylbenzylcarbamoyl)-piperazin-1-yl]-6-(4-fluorophenyl)-pyrido[3,2-d]pyrimidine (example 681),
2-chloromethyl-4-[4-(N-2-methylbenzylcarbamoyl)-piperazin-1-yl]-6-(4-fluorophenyl)-pyrido[3,2-d]pyrimidine (example 682),
2-chloromethyl-4-[4-(N-4-methylbenzylcarbamoyl)-piperazin-1-yl]-6-(4-fluorophenyl)-pyrido[3,2-d]pyrimidine (example 683),
2-chloromethyl-4-[4-(N-2-methoxybenzylcarbamoyl)-piperazin-1-yl]-6-(4-fluorophenyl)-pyrido[3,2-d]pyrimidine (example 684),
2-chloromethyl-4-[4-(N-3-methoxybenzylcarbamoyl)-piperazin-1-yl]-6-(4-fluorophenyl)-pyrido[3,2-d]pyrimidine (example 685),
2-chloromethyl-4-[4-(N-4-methoxybenzylcarbamoyl)-piperazin-1-yl]-6-(4-fluorophenyl)-pyrido[3,2-d]pyrimidine (example 686),
2-chloromethyl-4-[4-(N-2-fluorobenzylcarbamoyl)-piperazin-1-yl]-6-(4-fluorophenyl)-pyrido[3,2-d]pyrimidine (example 687),
2-chloromethyl-4-[4-(N-3-fluorobenzylcarbamoyl)-piperazin-1-yl]-6-(4-fluorophenyl)-pyrido[3,2-d]pyrimidine (example 688),
2-chloromethyl-4-[4-(N-4-fluorobenzylcarbamoyl)-piperazin-1-yl]-6-(4-fluorophenyl)-pyrido[3,2-d]pyrimidine (example 689),
2-chloromethyl-4-[4-(N-2-chlorobenzylcarbamoyl)-piperazin-1-yl]-6-(4-fluorophenyl)-pyrido[3,2-d]pyrimidine (example 690),
2-chloromethyl-4-[4-(N-3-chlorobenzylcarbamoyl)-piperazin-1-yl]-6-(4-fluorophenyl)-pyrido[3,2-d]pyrimidine (example 691),
2-chloromethyl-4-[4-(N-4-chlorobenzylcarbamoyl)-piperazin-1-yl]-6-(4-fluorophenyl)-pyrido[3,2-d]pyrimidine (example 692),
2-chloromethyl-4-[4-(N-methylthiocarbamoyl)-piperazin-1-yl]-6-(4-fluorophenyl)-pyrido[3,2-d]pyrimidine (example 693),
2-chloromethyl-4-[4-(N-ethylthiocarbamoyl)-piperazin-1-yl]-6-(4-fluorophenyl)-pyrido[3,2-d]pyrimidine (example 694),
2-chloromethyl-4-[4-(N-propylthiocarbamoyl)-piperazin-1-yl]-6-(4-fluorophenyl)-pyrido[3,2-d]pyrimidine (example 695),
2-chloromethyl-4-[4-(N-butylthiocarbamoyl)-piperazin-1-yl]-6-(4-fluorophenyl)-pyrido[3,2-d]pyrimidine (example 696),
2-chloromethyl-4-[4-(N-pentylthiocarbamoyl)-piperazin-1-yl]-6-(4-fluorophenyl)-pyrido[3,2-d]pyrimidine (example 697),
2-chloromethyl-4-[4-(N-hexylthiocarbamoyl)-piperazin-1-yl]-6-(4-fluorophenyl)-pyrido[3,2-d]pyrimidine (example 698),
2-chloromethyl-4-[4-(N-octylthiocarbamoyl)-piperazin-1-yl]-6-(4-fluorophenyl)-pyrido[3,2-d]pyrimidine (example 699),
2-chloromethyl-4-[4-(N-cyclopentylthiocarbamoyl)-piperazin-1-yl]-6-(4-fluorophenyl)-pyrido[3,2-d]pyrimidine (example 700),
2-chloromethyl-4-[4-(N-cyclohexylthiocarbamoyl)-piperazin-1-yl]-6-(4-fluorophenyl)-pyrido[3,2-d]pyrimidine (example 701),
2-chloromethyl-4-[4-(N-benzylthiocarbamoyl)-piperazin-1-yl]-6-(4-fluorophenyl)-pyrido[3,2-d]pyrimidine (example 702),
2-chloromethyl-4-[4-(N-D-α-methylbenzylthiocarbamoyl)-piperazin-1-yl]-6-(4-fluorophenyl)-pyrido[3,2-d]pyrimidine (example 703),
2-chloromethyl-4-[4-(N-L-α-methylbenzylthiocarbamoyl)-piperazin-1-yl]-6-(4-fluorophenyl)-pyrido[3,2-d]pyrimidine (example 704),
2-chloromethyl-4-[4-(N-2-methylbenzylthiocarbamoyl)-piperazin-1-yl]-6-(4-fluorophenyl)-pyrido[3,2-d]pyrimidine (example 705),
2-chloromethyl-4-[4-(N-3-methylbenzylthiocarbamoyl)-piperazin-1-yl]-6-(4-fluorophenyl)-pyrido[3,2-d]pyrimidine (example 706),
2-chloromethyl-4-[4-(N-4-methylbenzylthiocarbamoyl)-piperazin-1-yl]-6-(4-fluorophenyl)-pyrido[3,2-d]pyrimidine (example 707),
2-chloromethyl-4-[4-(N-4-methoxybenzylthiocarbamoyl)-piperazin-1-yl]-6-(4-fluorophenyl)-pyrido[3,2-d]pyrimidine (example 708),
2-chloromethyl-4-[4-(N-2-fluorobenzylthiocarbamoyl)-piperazin-1-yl]-6-(4-fluorophenyl)-pyrido[3,2-d]pyrimidine (example 709),
2-chloromethyl-4-[4-(N-3-fluorobenzylthiocarbamoyl)-piperazin-1-yl]-6-(4-fluorophenyl)-pyrido[3,2-d]pyrimidine (example 710),
2-chloromethyl-4-[4-(N-4-fluorobenzylthiocarbamoyl)-piperazin-1-yl]-6-(4-fluorophenyl)-pyrido[3,2-d]pyrimidine (example 711),
2-chloromethyl-4-[4-(N-2-chlorobenzylthiocarbamoyl)-piperazin-1-yl]-6-(4-fluorophenyl)-pyrido[3,2-d]pyrimidine (example 712),
2-chloromethyl-4-[4-(N-3-chlorobenzylthiocarbamoyl)-piperazin-1-yl]-6-(4-fluorophenyl)-pyrido[3,2-d]pyrimidine (example 713), and
2-chloromethyl-4-[4-(N-4-chlorobenzylthiocarbamoyl)-piperazin-1-yl]-6-(4-fluorophenyl)-pyrido[3,2-d]pyrimidine (example 714).

### EXAMPLES 715-744

According to the synthetic procedures as described above, the following compounds of the present invention are obtained:
4-[6-(4-Fluoro-phenyl)-2-imidazol-1-ylmethyl-pyrido[3,2-d]pyrimidin-4-yl]-piperazine-1-carboxylic acid m-tolylamide (example 715).
4-[6-(4-Fluoro-phenyl)-2-[1,2,4]triazol-1-ylmethyl-pyrido[3,2-d]pyrimidin-4-yl]-piperazine-1-carboxylic acid m-tolylamide (example 716)
4-[6-(4-Fluoro-phenyl)-2-pyrrolidin-1-ylmethyl-pyrido[3,2-d]pyrimidin-4-yl]-piperazine-1-carboxylic acid m-tolylamide (example 717).
4-[6-(4-Fluoro-phenyl)-2-[1,2,3]triazol-1-ylmethyl-pyrido[3,2-d]pyrimidin-4-yl]-piperazine-1-carboxylic acid m-tolylamide (example 718).
4-[6-(4-Fluoro-phenyl)-2-pyrazol-1-ylmethyl-pyrido[3,2-d]pyrimidin-4-yl]-piperazine-1-carboxylic acid m-tolylamide (example 719).
2-(4-Chloro-phenoxy)-N-[4-{4-[2-(4-chloro-phenoxy)-acetyl]-piperazin-1-yl}-6-(4-fluoro-phenyl)-pyrido[3,2-d]pyrimidin-2-yl]-acetamide (example 720).
4-[2-(N′,N′-Dimethyl-hydrazinomethyl)-6-(4-fluoro-phenyl)-pyrido[3,2-d]pyrimidin-4-yl]-piperazine-1-carboxylic acid m-tolylamide (example 721).
4-(4-tert-Butoxycarbonyl-piperazin-1-yl)-6-(4-fluoro-phenyl)-pyrido[3,2-d]pyrimidine-2-carboxylic acid ethyl ester (example 722).
4-{4-[2-(4-Chloro-phenoxy)-acetyl]-piperazin-1-yl}-6-(4-fluoro-phenyl)-pyrido[3,2-d]pyrimidine-2-carboxylic acid ethyl ester (example 723).
4-{4-[2-(4-Chloro-phenoxy)-acetyl]-piperazin-1-yl}-6-(4-fluoro-phenyl)-pyrido[3,2-d]pyrimidine-2-carboxylic acid amide (example 724).
4-{4-[2-(4-Chloro-phenoxy)-acetyl]-piperazin-1-yl}-6-(4-fluoro-phenyl)-pyrido[3,2-d]pyrimidine-2-carboxylic acid methylamide (example 725).
4-[2-Cyanomethyl-6-(4-fluoro-phenyl)-pyrido[3,2-d]pyrimidin-4-yl]-piperazine-1-carboxylic acid m-tolylamide (example 726).
4-{4-[2-(4-Chloro-phenoxy)-acetyl]-piperazin-1-yl}6-(4-fluoro-phenyl)-pyrido[3,2-d]pyrimidine-2-carboxylic acid propylamide (example 727).
4-{4-[2-(4-Chloro-phenoxy)-acetyl]-piperazin-1-yl}-6-(4-fluoro-phenyl)-pyrido[3,2-d]pyrimidine-2-carboxylic acid (2-methoxy-ethyl)-amide (example 728).
4{4-[2-(4-Chloro-phenoxy)-acetyl]-piperazin-1-yl}-6-(4-fluoro-phenyl)-pyrido[3,2-d]pyrimidine-2-carboxylic acid dimethylamide (example 729).
2-(4-Chloro-phenoxy)-1-{4-[6-(4-fluoro-phenyl)-2-(pyrrolidine-1-carbonyl)-pyrido[3,2-d]pyrimidin-4-yl]-piperazin-1-yl}-ethanone (example 730).
4-{4-[2-(4-Chloro-phenoxy)-acetyl]-piperazin-1-yl}-6-(4-fluoro-phenyl)-pyrido[3,2-d]pyrimidine-2-carboxylic acid cyclopropylamide (example 731).
4-(2-{4-[2-Amino-6-(4-fluoro-phenyl)-pyrido[3,2-d]pyrimidin-4-yl]-piperazin-1-yl}-2-oxo-ethoxy)-benzoic acid methyl ester (example 732).
4-{4-[2-(4-Chloro-phenoxy)-acetyl]-piperazin-1-yl}-6-(4-fluoro-phenyl)-pyrido[3,2-d]pyrimidine-2-carboxylic acid (example 733).
4-{4-[2-(4-Chloro-phenoxy)-acetyl]-piperazin-1-yl}-6-(4-fluoro-phenyl)-pyrido[3,2-d]pyrimidine-2-carbonitrile (example 734).
4-(6-Pyridin-3-yl-pyrido[3,2-d]pyrimidin-4-yl)-piperazine-1-carboxylic acid m-tolylamide (example 735).
4-(6-Thiophen-3-yl-pyrido[3,2-d]pyrimidin-4-yl)-piperazine-1-carboxylic acid m-tolylamide (example 736)
4-[2-Amino-6-(4-fluoro-phenyl)-pyrido[3,2-d]pyrimidin-4-yl]-piperazine-1,3-dicarboxylic acid 3-tert-butylamide 1-m-tolylamide (example 737).
4-(6-Chloro-pyrido[3,2-d]pyrimidin-4-yl)-piperazine-1-carboxylic acid m-tolylamide (example 738).
4-(6-Pyridin-4-yl-pyrido[3,2-d]pyrimidin-4-yl)-piperazine-1-carboxylic acid m-tolylamide (example 739).
4-[6-(2-Chloro-phenyl)-pyrido[3,2-d]pyrimidin-4-yl]-piperazine-1-carboxylic acid m-tolylamide (example 740).
4-(6-o-Tolyl-pyrido[3,2-d]pyrimidin-4-yl)-piperazine-1-carboxylic acid m-tolylamide (example 741).
4-(6-Furan-3-yl-pyrido[3,2-d]pyrimidin-4-yl)-piperazine-1-carboxylic acid m-tolylamide (example 742).
4-[6-(2-Cyano-phenyl)-pyrido[3,2-d]pyrimidin-4-yl]-piperazine-1-carboxylic acid (4-chloro-2-trifluoromethyl-phenyl)-amide (example 743).
4-[6-(2-Cyano-phenyl)-pyrido[3,2-d]pyrimidin-4-yl]-piperazine-1-carboxylic acid (4-tert-butyl-phenyl)-amide (example 744).

1. A pyrido(3,2-d)pyrimidine derivative having the general formula: 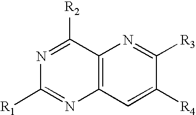
wherein:
R₁ is selected from the group consisting of hydrogen, halogen, cyano, carboxylic acid, acyl, thioacyl, alkoxycarbonyl, acyloxy, carbonate, carbamate, C₁₋₇ alkyl, aryl, amino, acetamido, N-protected amino, (mono- or di) C₁₋₇ alkylamino, (mono- or di) arylamino, (mono- or di) C₃₋₁₀ cycloalkylamino, (mono- or di) hydroxy C₁₋₇ alkylamino, (mono- or di) C₁₋₄ alkyl-arylamino, mercapto C₁₋₇ alkyl, C₁₋₇ alkyloxy, and groups of the formula R₆—NR₇R₁₂, wherein R₆ is a bond or C₁₋₃ alkylene, wherein R₇ and R₁₂ are independently selected from the group consisting of hydrogen, C₁₋₇ alkyl, C₂₋₇ alkenyl, C₂₋₇ alkynyl, aryl, arylalkyl, C₃₋₁₀ cycloalkyl and heteroaryl, or wherein R₇ and R₁₂ together form a heterocycle,
R₂ is selected from the group consisting of (mono- or di-) C₁₋₁₂ alkylamino; monoarylamino; diarylamino; (mono- or di-) C₃₋₁₀ cycloalkylamino; (mono- or di-) hydroxyC₁₋₇ alkylamino; (mono- or di-) C₁₋₄ alkylarylamino; (mono- or di-) arylC₁₋₄ alkylamino; morpholinyl; mercapto C₁₋₇ alkyl; C₁₋₇ alkoxy, homopiperazinyl and piperazinyl, wherein said homopiperazinyl or piperazinyl is optionally N-substituted with a substituent R₅ selected from the group consisting of formyl, acyl, thioacyl, amide, thioamide, sulfonyl, sulfinyl, carboxylate, thiocarboxylate, amino-substituted acyl, alkoxyalkyl, C₃₋₁₀ cycloalkyl-alkyl, C₃₋₁₀ cycloalkyl, dialkylaminoalkyl, heterocyclic-substituted alkyl, acyl-substituted alkyl, thioacyl-substituted alkyl, amido-substituted alkyl, thioamido-substituted alkyl, carboxylato-substituted alkyl, thiocarboxylato-substituted alkyl, (amino-substituted acyl)alkyl, heterocyclic, carboxylic acid ester, ω-cyanoalkyl, ω-carboxylic ester-alkyl, halo C₁₋₇ alkyl, C₂₋₇ alkenyl, C₂₋₇ alkynyl, arylalkenyl, aryloxyalkyl, arylalkyl and aryl, wherein the aryl moiety of each of said arylalkenyl, aryloxyalkyl, arylalkyl and aryl radicals is optionally substituted with one or more substituents independently selected from the group consisting of halogen, C₁₋₇ alkyl, C₂₋₇ alkenyl, C₂₋₇ alkynyl, halo C₁₋₇ alkyl, nitro, hydroxyl, sulfhydryl, amino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thio-heterocyclic, arylalkylthio, heterocyclic-substituted alkylthio, formyl, carbamoyl, thiocarbamoyl, ureido, thioureido, sulfonamido, hydroxylamino, alkoxy-amino, mercaptoamino, thioalkylamino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazino, alkylhydrazino and phenylhydrazino;
R₃ and R₄ are independently selected from the group consisting of hydrogen, heteroaryl and aryl groups, wherein said heteroaryl or aryl groups are optionally substituted with one or more substituents selected from the group consisting of halogen, C₁₋₇ alkyl, C₂₋₇ alkenyl, C₂₋₇ alkynyl, halo C₁₋₇ alkyl, nitro, hydroxyl, sulfhydryl, amino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thio-heterocyclic, arylalkylthio, heterocyclic-substituted alkylthio, formyl, carbamoyl, thiocarbamoyl, ureido, thioureido, sulfonamido, hydroxylamino, alkoxy-amino, mercaptoamino, thioalkylamino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazino, alkylhydrazino and phenylhydrazino, provided that R₃ and R₄ are not both hydrogen, and further provided that R₄ is hydrogen when R₂ is monoarylamino,
or a pharmaceutical acceptable addition salt or a stereochemical isomeric form thereof or a N-oxide thereof or a solvate thereof.

2. A pyrido(3,2-d)pyrimidine derivative according to claim 1, wherein R₄ is hydrogen.

3. A pyrido(3,2-d)pyrimidine derivative according to claim 1, wherein R₁ is not hydrogen.

4. A pyrido(3,2-d)pyrimidine derivative according to claim 1, wherein R₁ is amino or acetamido.

5. A pyrido(3,2-d)pyrimidine derivative according to claim 1, wherein R₁ is amino or acetamido, and further wherein R₃ is a substituted aryl group.

6. A pyrido(3,2-d)pyrimidine derivative according to claim 1, wherein R₁ is amino or acetamido, wherein R₃ is a substituted aryl group and wherein R₄ is hydrogen.

7. A pyrido(3,2-d)pyrimidine derivative according to claim 1, wherein R₂ is a piperazin-1-yl group, said group being optionally substituted in the 4 position with a substituent R₅, wherein R₅ is selected from the group consisting of:
COR₈ wherein R₈ is selected from hydrogen; C₁₋₇ alkyl; C₃₋₁₀ cycloalkyl; aryl optionally substituted with one or more substituents selected from the group consisting of halogen, C₁₋₇ alkyl, cyano and C₁₋₇ alkoxy; heterocyclic optionally substituted with one or more halogen atoms; arylalkyl; aryloxyalkyl; arylalkoxyalkyl; alkoxyalkyl; arylalkoxy; aryloxy; arylalkenyl; heterocyclic-substituted alkyl; alkylamino, arylamino and alkylarylamino;
CSR₉, wherein R₉ is selected from the group consisting of alkylamino and aryloxy;
SO₂R₁₀, wherein R₁₀ is selected from the group consisting of aryl and arylalkyl; and
R₁₁, wherein R₁₁ is selected from the group consisting of C₁₋₇ alkyl, aryl, arylalkyl, arylalkenyl, alkoxyalkyl, heterocyclic-substituted alkyl, cycloalkylalkyl, hetero-cyclic, C₃₋₁₀ cycloalkyl, alkylaminoalkyl, aryloxyalkyl, alkoxyaryl, ω-cyanoalkyl, ω-carboxylatoalkyl and carboxamidoalkyl.

8. A pyrido(3,2-d)pyrimidine derivative having the general formula: 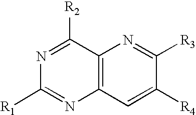
wherein:
R₁ is selected from the group consisting of hydrogen, halogen, C₁₋₇ alkyl, aryl, amino, acetamido, N-protected amino, (mono- or di) C₁₋₇ alkylamino, (mono- or di) C₁₋₄ alkyl-arylamino, C₁₋₇ alkyloxy, and groups of the formula R₆—NR₇R₁₂, wherein R₆ is a bond or C₁₋₃ alkylene, wherein R₇ and R₁₂ together form a heterocycle,
R₂ is selected from the group consisting of (mono- or di-) C₁₋₁₂ alkylamino; monoarylamino; diarylamino; (mono- or di-) C₁₋₄ alkylarylamino; (mono- or di-) arylC₁₋₄ alkylamino; morpholinyl; mercapto C₁₋₇ alkyl; C₁₋₇ alkoxy; homopiperazinyl and piperazinyl, wherein said homopiperazinyl or piperazinyl is optionally N-substituted, with a substituent R₅ selected from the group consisting of acyl, heterocyclic-substituted alkyl, heterocyclic, aryloxyalkyl, arylalkyl and aryl, wherein the aryl moiety of each of said arylalkenyl, aryloxyalkyl, arylalkyl and aryl radicals is optionally substituted with one or more substituents independently selected from the group consisting of halogen and C₁₋₇ alkyl;
R₃ and R₄ are independently selected from the group consisting of hydrogen, heteroaryl and aryl groups, wherein said or aryl, groups are optionally substituted with one or more substituents selected from the group consisting of halogen, C₁₋₇ alkyl, hydroxyl, C₁₋₇ alkoxy, C₃₋₁₀ cycloalkoxy,
or a pharmaceutical acceptable addition salt or a stereochemical isomeric form thereof
or a N-oxide thereof or a solvate thereof.

9. A pyrido[3,2-d]pyrimidine derivative selected from the group consisting of:
2-chloro-4-(4-[3-methylphenyl)amino]carbonyl]piperazin-1-yl)-6-(3,4-dimethoxyphenyl)pyrido[3,2-d]pyrimidine
2-dimethylamino-4-(4-[3-methylphenyl)amino]carbonyl]piperazin-1-yl)-6-(3,4-dimethoxyphenyl)pyrido[3,2-d]pyrimidine
2-[(N-hydroxyethyl)morpholino]-4-(4-[3-methylphenyl)amino]carbonyl]piperazin-1-yl)-6-(3,4-dimethoxyphenyl)pyrido[3,2-d]pyrimidine
2-(1-methyl-2-pyrrolidino-ethoxy)-4-(4-[3-methylphenyl)amino]carbonyl]piperazin-1-yl)-6-(3,4-dimethoxyphenyl)pyrido[3,2-d]pyrimidine
2-(2-phenoxyethoxy)-4-(4-[3-methylphenyl)amino]carbonyl]piperazin-1-yl)-6-(3,4-dimethoxyphenyl)pyrido[3,2-d]pyrimidine
2-phenyl-4-(4-[3-methylphenyl)amino]carbonyl]piperazin-1-yl)-6-(3,4-dimethoxyphenyl)pyrido[3,2-d]pyrimidine
2-acetamido-4-(1,2,4-triazolyl)-6-(3,4-dimethoxyphenyl)-pyrido[3,2-d]pyrimidine
2-amino-4-[4-(ethoxycarbonyl)piperidin-1-yl]-6-(3,4-dimethoxyphenyl)pyrido[3,2-d]pyrimidine
2-amino-4-[3,4-(methylenedioxy)aniline]-6-(3,4-dimethoxyphenyl)pyrido[3,2-d]pyrimidine
2-amino-4-(N-methyl-piperazino)-6-(3,4-dimethoxyphenyl)-pyrido[3,2-d]pyrimidine
2-amino-4-(thienyl-2-methylamino)-6-(3,4-dimethoxyphenyl)-pyrido[3,2-d]pyrimidine-2,4-diamine
2-amino-4-[4-(2-aminoethyl)morpholino]-6-(3,4-dimethoxyphenyl)-pyrido[3,2-d]pyrimidine
2-amino-4-[2-(aminomethyl)pyridino]-6-(3,4-dimethoxyphenyl)-pyrido[3,2-d]pyrimidine
2,4-diamino-6-(3,4-dimethoxyphenyl)pyrido[3,2-d]pyrimidine
2-amino-4-(4-{[(3-methylphenyl)amino]carbonyl}piperazin-1-yl)-6-(3,4-dimethoxyphenyl)-pyrido[3,2-d]pyrimidine
2-amino-4-[2-(piperazin-1-yl)-acetic acid N-(2-thiazolyl)-amide]-6-(3,4-dimethoxyphenyl)-pyrido[3,2-d]pyrimidine
2-morpholino-4-[(N-3-methyl-phenylcarbamoyl-piperazin-1-yl]-6-(3,4-dimethoxyphenyl)-pyrido[3,2-d]pyrimidine
2-butoxy-4-[(N-3-methyl-phenylcarbamoyl)-piperazin-1-yl]-6-(3,4-dimethoxyphenyl)-pyrido[3,2-d]pyrimidine
2-methoxy-4-[(N-3-methyl-phenylcarbamoyl)-piperazin-1-yl]-6-(3,4-dimethoxy-phenyl)-pyrido[3,2-d]pyrimidine
2-(p-tolylamino)-4-[(N-3-methylphenylcarbamoyl)-piperazin-1-yl]-6-(3,4-dimethoxy-phenyl)-pyrido[3,2-d]pyrimidine
2-[(3-chloro-4-fluoro-anilino)-4-[(N-3-methyl-phenylcarbamoyl)-piperazin-1-yl]-6-(3,4-dimethoxyphenyl)-pyrido[3,2-d]pyrimidine
2,4-diamino-6-(4-hydroxy-3-methoxyphenyl)-pyrido[3,2-d]pyrimidine
2,4-diamino-6-(3-chloro-4-methoxyphenyl)-pyrido[3,2-d]pyrimidine
2-amino-4-[(N-4-fluoro-phenyl-carbamoyl)-piperazin-1-yl]-6-(4-hydroxy-3-methoxy-phenyl)-pyrido[3,2-d]pyrimidine
4-(4-methyl-phenyl-piperazin-1-yl)-6-(3,4-dimethoxy-phenyl)-pyrido[3,2-d]pyrimidine
2-acetamido-4-(N-piperazin-1-yl)-6-(1,4-benzodioxane-phenyl)pyrido[3,2-d]pyrimidine
2-amino-4-(morpholino)-6-(1,4-benzodioxane-phenyl)pyrido[3,2-d]pyrimidine
2-acetamido-4-(1,2,4-triazolyl)-6-(3,4-dichloro-phenyl)pyrido[3,2-d]pyrimidine
2-acetamido-4-(1,2,4-triazolyl)-6-(4-fluoro-phenyl)-pyrido[3,2-d]pyrimidine
2-acetamido-4-(1,2,4-triazolyl)-6-(1,4-benzodioxane)-pyrido[3,2-d]pyrimidine
2-acetamido-4-(1,2,4-triazolyl)-6-(3,4-methylenedioxyphenyl)-pyrido[3,2-d]pyrimidine
2-amino-4-[(N-4-chloro-benzylcarbamoyl)-piperazin-1-yl]-6-(4-fluorophenyl)-pyrido[3,2-d]pyrimidine
2-amino-4-[2-(piperazin-1-yl acetic acid N-(2-thiazolyl)-amide)]-6-3,4-methylenedioxyphenyl)-pyrido[3,2-d]pyrimidine
2-amino-4-[N-(3-methyl-phenyl-carbamoyl)-piperazin-1-yl]-6-(3,4-methylenedioxyphenyl)-pyrido[3,2-d]pyrimidine
2-amino-4-[N-(3-methyl-phenyl-carbamoyl)-piperazin-1-yl]-6-(3,4-dichlorophenyl)-pyrido[3,2-d]pyrimidine
2-amino-4-[N-(3-methyl-phenyl-carbamoyl)-piperazin-1-yl]-6-(1,4-benzodioxane)-pyrido[3,2-d]pyrimidine
2-amino-4-[2-(piperazin-1-yl acetic acid N-(2-thiazolyl)-amide]-6-(1,4-benzodioxane)-pyrido[3,2-d]pyrimidine
2-amino-4-[2-(piperazin-1-yl acetic acid N-(2-thiazolyl)-amide]-6-(3,4-dichlorophenyl)-pyrido[3,2-d]pyrimidine
2-acetamido-4-[(S)-3-(Boc-amino)pyrrolidine]-6-chloro-pyrido[3,2-d]pyrimidine
2-amino-4-[(S)-3-(Boc-amino)pyrrolidine]-6-chloro-pyrido[3,2-d]pyrimidine
2-amino-4-[(S)-3-(Boc-amino)pyrrolidine]-6-(3,4-dimethoxyphenyl)-pyrido[3,2-d]pyrimidine
2-amino-4-[(S)-3-(amino)pyrrolidine]-6-(3,4-dimethoxyphenyl)-pyrido[3,2-d]pyrimidine
2-amino-4-[3-(S)-4-chloro-phenoxy-acetyl-amino)-pyrrolidin-1-yl]-6-(3,4-dimethoxyphenyl)-pyrido[3,2-d]pyrimidine
2-amino-4-[3-(S)-3-methyl phenyl carbamoyl pyrrolidin-1-yl]-6-(3,4-dimethoxyphenyl)-pyrido[3,2-d]pyrimidine
2-acetamido-4-[(R)-3-Boc-aminopyrrolidin-1-yl]-6-chloro pyrido[3,2-d]pyrimidine
2-amino-4-[(R)-3-Boc-aminopyrrolidin-1-yl)]-6-(3,4-dimethoxyphenyl)-pyrido[3,2-d]pyrimidine
2-amino-4-[(1-Boc-piperidin-4-yl)amino]-6-(3,4-dimethoxyphenyl)-pyrido[3,2-d]pyrimidine
2-amino-4-[(1-Boc-piperidin-3-yl)amino]-6-(3,4-dimethoxyphenyl)-pyrido[3,2-d]pyrimidine
2-amino-4-[(1-Cbz-piperidin-3-yl)amino]-6-(3,4-dimethoxyphenyl)-pyrido[3,2-d]pyrimidine
2-amino-4-[(R)-3-aminopyrrolidin-1-yl]-6-(3,4-dimethoxy-phenyl)-pyrido[3,2-d]pyrimidine
2-amino-4-[3-(R)-(3-methylphenylcarbamoyl)-pyrrolidin-1-yl]-6-(3,4-dimethoxyphenyl)-pyrido[3,2-d]pyrimidine
2-amino-4-[(3-methylphenylcarbamoyl)-ethylenediamine-1-N-yl]-6-(3,4-dimethoxyphenyl)-pyrido[3,2-d]pyrimidine
2-amino-4-[(3-methylphenylcarbamoyl)-3-aminopropane-amino-1-N-yl]-6-(3,4-dimethoxyphenyl)-pyrido[3,2-d]pyrimidine
2-amino-4-[1-(3-methylphenylcarbamoyl)piperidin-4-yl)amino]-6-(3,4-dimethoxyphenyl)-pyrido[3,2-d]pyrimidine
2-amino-4-[(3-methylphenylcarbamoyl)piperidin-3-yl)amino)-6-(3,4-dimethoxyphenyl]-pyrido[3,2-d]pyrimidine
2-amino-4-[2-(4-chlorophenoxy-acetyl-ethylenediamine-1-N-yl]-6-(3,4-dimethoxyphenyl)-pyrido[3,2-d]pyrimidine
2-amino-4-[3-N-(4-chlorophenoxy-acetyl)-3-amino-propane-amine-1-N-yl]-6-(3,4-dimethoxyphenyl)-pyrido[3,2-d]pyrimidine
2-amino-4-[(3-(R)-(4-chlorophenoxyacetyl-amino)-pyrrolidin-1-yl]-6-(3,4-dimethoxyphenyl)-pyrido[3,2-d]pyrimidine
2-amino-4-[(3-carboxylic acid isobutylamide)-piperidin-1-yl]-6-(3,4-dimethoxyphenyl)-pyrido[3,2-d]pyrimidine
2-amino-4-(4-chlorophenyl-4-hydroxypiperidin-1-yl)-6-(3,4-dimethoxy-phenyl)-pyrido[3,2-d]pyrimidine
2-amino-4-[4-(N-2-phenylethylacetamid-2-yl)piperazin-1-yl]-6-(3,4-dimethoxyphenyl)-pyrido[3,2-d]pyrimidine
2-amino-4-[3-(4-acetylpiperazin-1-yl)-propan-3-one-1-yl-amino]-6-(3,4-dimethoxyphenyl)-pyrido[3,2-d]pyrimidine
2-amino-4-(N-pyrrolidinyl-acetamid-2-yl-piperazin-1-yl)-6-(3,4-dimethoxyphenyl)pyrido[3,2-d]pyrimidine
2-amino-4-(N-pyridinylacetamid-2-yl-piperazin-1-yl)-6-(3,4-dimethoxyphenyl)-pyrido[3,2-d]pyrimidine
2-amino-4-[N-(piperazino)-acetyl-morpholino]-6-(3,4-dimethoxyphenyl)-pyrido[3,2-d]pyrimidine
2-amino-4-[2-amino-1-(4-methyl-piperazin-1-yl)-ethanone]-6-(3,4-dimethoxyphenyl)-pyrido[3,2-d]pyrimidine
2-acetamido-4-[(N-pyridin-3-yl-acetamid)-2-yl-piperazin-1-yl]-6-(3,4-dimethoxyphenyl)-pyrido[3,2-d]pyrimidine
2-acetamido-4-[(N-methyl-N-phenylacetamid)-2-yl-piperazin-1-yl]-6-(3,4-dimethoxyphenyl)-pyrido[3,2-d]pyrimidine
2-acetamido-4-benzyloxy-6-chloro-pyrido[3,2-d]pyrimidine
2-amino-4-benzyloxy-6-(o-fluorophenyl)-pyrido[3,2-d]pyrimidine
2-amino-4-benzyloxy-6-(m-fluorophenyl)-pyrido[3,2-d]pyrimidine
2-amino-4-benzyloxy-6-(p-fluorophenyl)-pyrido[3,2-d]pyrimidine
2-amino-4-(1,8-diaminooctyl)-6-(3,4-dimethoxyphenyl)pyrido[3,2-d]pyrimidine
2-amino-4-(1,9-diaminononyl)-6-(3,4-dimethoxyphenyl)pyrido[3,2-d]pyrimidine
2-amino-4-(1,10-diaminodecyl)-6-(3,4-dimethoxyphenyl)pyrido[3,2-d]pyrimidine
2-amino-4-(1,12-diaminododecyl)-6-(3,4-dimethoxyphenyl)pyrido[3,2-d]pyrimidine
2-acetamido-4-(1,2,4-triazolyl)-6-(3-methyl-4-methoxyphenyl)pyrido-[3,2-d]pyrimidine
2-acetamido-4-(1,2,4-triazolyl)-6-(3-chloro-4-methoxy-phenyl)pyrido-[3,2-d]pyrimidine
2-acetamido-4-(1,2,4-triazolyl)-6-(3-chloro-4-ethoxy-phenyl)pyrido-[3,2-d]pyrimidine
2-acetamido-4-(1,2,4-triazolyl)-6-(3-fluoro-4-ethoxy-phenyl)pyrido-[3,2-d]pyrimidine
2-acetamido-4-(1,2,4-triazolyl)-6-(3-methyl-4-fluoro-phenyl)pyrido-[3,2-d]pyrimidine
2-acetamido-4-(1,2,4-triazolyl)-6-(3,4-dichloro-phenyl)pyrido-[3,2-o]pyrimidine
2-acetamido-4-(1,2,4-triazolyl)-6-(3,4-(methylenedioxy)phenyl)pyrido[3,2-d]pyrimidine
2-acetamido-4-(1,2,4-triazolyl)-6-(1,4-benzodioxane-phenyl)pyrido[3,2-o]pyrimidin-4(3H)-one

10. A pyrido(3,2-d)pyrimidine derivative according to claim 1, being selected from the group consisting of:
4-[(2-phenoxyethyl)-piperazin-1-yl]-6-(3,4-dimethoxyphenyl)-pyrido[3,2-d]pyrimidine,
4-(4-[3-methylphenyl)amino]carbonyl]piperazin-1-yl)-6-(3,4-dimethoxyphenyl)-pyrido[3,2-d]pyrimidine,
2-methyl-4-(4-[3-methylphenyl)amino]carbonyl]piperazin-1-yl)-6-(3,4-dimethoxy-phenyl)-pyrido[3,2-d]pyrimidine,
2-dimethylamino-4-(4-[3-methylphenyl)amino]carbonyl]piperazin-1-yl)-6-(3,4-dimethoxyphenyl)pyrido[3,2-d]pyrimidine,
2-[(N-hydroxyethyl)morpholino]-4-(4-[3-methylphenyl)amino]carbonyl]piperazin-1-yl)-6-(3,4-dimethoxyphenyl)pyrido[3,2-d]pyrimidine,
2-(1-methyl-2-pyrrolidino-ethoxy)-4-(4-[3-methylphenyl)amino]carbonyl]piperazin-1-yl)-6-(3,4-dimethoxyphenyl)pyrido[3,2-d]pyrimidine,
2-(2-phenoxyethoxy)-4-(4-[3-methylphenyl)amino]carbonyl]piperazin-1-yl)-6-(3,4-dimethoxyphenyl)pyrido[3,2-d]pyrimidine,
2-phenyl-4-(4-[3-methylphenyl)amino]carbonyl]piperazin-1-yl)-6-(3,4-dimethoxy-phenyl)pyrido[3,2-d]pyrimidine,
2-amino-4-morpholino-6-(3,4-dimethoxyphenyl)-pyrido[3,2-d]pyrimidine,
2-amino-4-isopropoxy-6-(3,4-dimethoxyphenyl)-pyrido[3,2-d]pyrimidine,
2-amino-4-phenoxyethoxy-6-(3,4-dimethoxyphenyl)-pyrido[3,2-d]pyrimidine,
2-amino-4-(4-[3-methylphenyl)amino]carbonyl]piperazin-1-yl)-6-(3,4-dimethoxy-phenyl)-pyrido[3,2-d]pyrimidine,
2-amino-4-[(4-carboxylic ethyl ester)-piperidin-1-yl]-6-(3,4-dimethoxyphenyl)pyrido[3,2-d]pyrimidine,
2-amino-4-(m-tolylamino)-6-(3,4-dimethoxyphenyl)-pyrido[3,2-d]pyrimidine,
2-amino-4-benzodioxolanylamino-6-(3,4-dimethoxyphenyl)-pyrido[3,2-d]pyrimidine,
2-amino-4-(m-bromophenylamino)-6-(3,4-dimethoxyphenyl)-pyrido[3,2-d]pyrimidine,
2-amino-4-(4-methylpiperazin-1-yl)-6-(3,4-dimethoxyphenyl)-pyrido[3,2-d]pyrimidine,
2-amino-4-(thien-2-ylmethyl)amino-6-(3,4-dimethoxyphenyl)-pyrido[3,2-d]pyrimidine,
2-amino-4-(2-N-morpholinoethyl)amino-6-(3,4-dimethoxyphenyl)-pyrido[3,2-d]pyrimidine,
2-amino-4-(2,2-dimethoxyethyl)amino-6-(3,4-dimethoxyphenyl)-pyrido[3,2-d]pyrimidine,
2-amino-4-(pyridin-2-ylmethyl)amino-6-(3,4-dimethoxyphenyl)-pyrido[3,2-d]pyrimidine,
2-amino-4-(2-chloro-5-methoxyphenyl)amino-6-(3,4-dimethoxyphenyl)-pyrido[3,2-d]pyrimidine,
2-amino-4-(4-aminocyclohexylamino)-6-(3,4-dimethoxyphenyl)-pyrido[3,2-d]pyrimidine,
2-N-morpholinylethoxy-4-(4-[3-methylphenyl)amino]carbonyl]piperazin-1-yl)-6-chloro-pyrido[3,2-d]pyrimidine,
2-chloro-4-(4-[3-methylphenyl)amino]carbonyl]piperazin-1-yl)-6-(3,4-dimethoxy-phenyl)-pyrido[3,2-d]pyrimidine,
4-[6-(4-Fluoro-phenyl)-2-imidazol-1-ylmethyl-pyrido[3,2-d]pyrimidin-4-yl]-piperazine-1-carboxylic acid m-tolylamide,
4-[6-(4-Fluoro-phenyl)-2-[1,2,4]triazol-1-ylmethyl-pyrido[3,2-d]pyrimidin-4-yl]-piperazine-1-carboxylic acid m-tolylamide,
4-[6-(4-Fluoro-phenyl)-2-pyrrolidin-1-ylmethyl-pyrido[3,2-d]pyrimidin-4-yl]-piperazine-1-carboxylic acid m-tolylamide,
4-[6-(4-Fluoro-phenyl)-2-[1,2,3]triazol-1-ylmethyl-pyrido[3,2-d]pyrimidin-4-y]-piperazine-1-carboxylic acid m-tolylamide,
4-[6-(4-Fluoro-phenyl)-2-pyrazol-1-ylmethyl-pyrido[3,2-d]pyrimidin-4-yl]-piperazine-1-carboxylic acid m-tolylamide,
2-(4-Chloro-phenoxy)-N-[4-{4-[2-(4-chloro-phenoxy)-acetyl]-piperazin-1-yl}-6-(4-fluoro-phenyl)-pyrido[3,2-d]pyrimidin-2-yl]-acetamide,
4-[2-(N′,N′-Dimethyl-hydrazinomethyl)-6-(4-fluoro-phenyl)-pyrido[3,2-d]pyrimidin-4-yl]-piperazine-1-carboxylic acid m-tolylamide,
4-(4-tert-Butoxycarbonyl-piperazin-1-yl)-6-(4-fluoro-phenyl)-pyrido[3,2-d]pyrimidine-2-carboxylic acid ethyl ester,
4-{4-[2-(4-Chloro-phenoxy)-acetyl]-piperazin-1-yl}-6-(4-fluoro-phenyl)-pyrido[3,2-d]pyrimidine-2-carboxylic acid ethyl ester,
4-{4-[2-(4-Chloro-phenoxy)-acetyl]-piperazin-1-yl}-6-(4-fluoro-phenyl)-pyrido[3,2-d]pyrimidine-2-carboxylic acid amide,
4-{4-[2-(4-Chloro-phenoxy)-acetyl]-piperazin-1-yl}-6-(4-fluoro-phenyl)-pyrido[3,2-d]pyrimidine-2-carboxylic acid methylamide,
4-[2-Cyanomethyl-6-(4-fluoro-phenyl)-pyrido[3,2-d]pyrimidin-4-yl]-piperazine-1-carboxylic acid m-tolylamide,
4-{4-[2-(4-Chloro-phenoxy)-acetyl]-piperazin-1-yl}-6-(4-fluoro-phenyl)-pyrido[3,2-d]pyrimidine-2-carboxylic acid propylamide,
4-{4-[2-(4-Chloro-phenoxy)-acetyl]-piperazin-1-yl}-6-(4-fluoro-phenyl)-pyrido[3,2-d]pyrimidine-2-carboxylic acid (2-methoxy-ethyl)-amide,
4-{4-[2-(4-Chloro-phenoxy)-acetyl]-piperazin-1-yl}-6-(4-fluoro-phenyl)-pyrido[3,2-d]pyrimidine-2-carboxylic acid dimethylamide,
2-(4-Chloro-phenoxy)-1-{4-[6-(4-fluoro-phenyl)-2-(pyrrolidine-1-carbonyl)-pyrido[3,2-d]pyrimidin-4-yl]-piperazin-1-yl}-ethanone,
4-{4-[2-(4-Chloro-phenoxy)-acetyl]-piperazin-1-yl}-6-(4-fluoro-phenyl)-pyrido[3,2-d]pyrimidine-2-carboxylic acid cyclopropylamide,
4-(2-{4-[2-Amino-6-(4-fluoro-phenyl)-pyrido[3,2-d]pyrimidin-4-yl]-piperazin-1-yl}2-oxo-ethoxy)-benzoic acid methyl ester,
4-{4-[2-(4-Chloro-phenoxy)-acetyl]-piperazin-1-yl}-6-(4-fluoro-phenyl)-pyrido[3,2-d]pyrimidine-2-carboxylic acid,
4-{4-[2-(4-Chloro-phenoxy)-acetyl]-piperazin-1-yl}-6-(4-fluoro-phenyl)-pyrido[3,2-d]pyrimidine-2-carbonitrile,
4-(6-Pyridin-3-yl-pyrido[3,2-d]pyrimidin-4-yl)-piperazine-1-carboxylic acid m-tolylamide,
4-(6-Thiophen-3-yl-pyrido[3,2-d]pyrimidin-4-yl)-piperazine-1-carboxylic acid m-tolylamide,
4-[2-Amino-6-(4-fluoro-phenyl)-pyrido[3,2-d]pyrimidin-4-yl]-piperazine-1,3-dicarboxylic acid 3-tert-butylamide 1-m-tolylamide,
4-(6-Chloro-pyrido[3,2-d]pyrimidin-4-yl)-piperazine-1-carboxylic acid m-tolylamide,
4-(6-Pyridin-4-yl-pyrido[3,2-d]pyrimidin-4-yl)-piperazine-1-carboxylic acid m-tolylamide,
4-[6-(2-Chloro-phenyl)-pyrido[3,2-d]pyrimidin-4-yl]-piperazine-1-carboxylic acid m-tolylamide,
4-(6-o-Tolyl-pyrido[3,2-d]pyrimidin-4-yl)-piperazine-1-carboxylic acid m-tolylamide,
4-(6-Furan-3-yl-pyrido[3,2-d]pyrimidin-4-yl)-piperazine-1-carboxylic acid m-tolylamide,
4-[6-(2-Cyano-phenyl)-pyrido[3,2-d]pyrimidin-4-yl]-piperazine-1-carboxylic acid (4-chloro-2-trifluoromethyl-phenyl)-amide, and
4-[6-(2-Cyano-phenyl)-pyrido[3,2-d]pyrimidin-4-yl]-piperazine-1-carboxylic acid (4-tert-butyl-phenyl)-amide.

11. A pharmaceutical composition comprising one or more pharmaceutically acceptable carriers and a pyrido(3,2-d)pyrimidine derivative having the general formula: 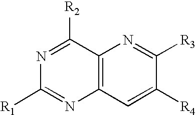
wherein:
R₁ is selected from the group consisting of hydrogen, halogen, cyano, carboxylic acid, acyl, thioacyl, alkoxycarbonyl, acyloxy, carbonate, carbamate, C₁₋₇ alkyl, aryl, amino, acetamido, N-protected amino, (mono- or di) C₁₋₇ alkylamino, (mono- or di) arylamino, (mono- or di) C₃₋₁₀ cycloalkylamino, (mono- or di) hydroxy C₁₋₇ alkylamino, (mono- or di) C₁₋₄ alkyl-arylamino, mercapto C₁₋₇ alkyl, C₁₋₇ alkyloxy, and groups of the formula R₆—NR₇R₁₂, wherein R₆ is a bond or C₁₋₃ alkylene, wherein R₇ and R₁₂ are independently selected from the group consisting of hydrogen, C₁₋₇ alkyl, C₂₋₇ alkenyl, C₂₋₇ alkynyl, aryl, arylalkyl, C₃₋₁₀ cycloalkyl and heteroaryl, or wherein R₇ and R₁₂ together form a heterocycle,
R₂ is selected from the group consisting of (mono- or di-) C₁₋₁₂ alkylamino; monoarylamino; diarylamino; (mono- or di-) C₃₋₁₀ cycloalkylamino; (mono- or di-) hydroxyC₁₋₇ alkylamino; (mono- or di-) C₁₋₄ alkylarylamino; (mono- or di-) aryl C₁₋₄ alkylamino; morpholinyl; mercapto C₁₋₇ alkyl; C₁₋₇ alkoxy, homopiperazinyl and piperazinyl, wherein said homopiperazinyl or piperazinyl is optionally N-substituted with a substituent R₅ selected from the group consisting of formyl, acyl, thioacyl, amide, thioamide, sulfonyl, sulfinyl, carboxylate, thiocarboxylate, amino-substituted acyl, alkoxyalkyl, C₃₋₁₀ cycloalkyl-alkyl, C₃₋₁₀ cycloalkyl, dialkylaminoalkyl, heterocyclic-substituted alkyl, acyl-substituted alkyl, thioacyl-substituted alkyl, amido-substituted alkyl, thioamido-substituted alkyl, carboxylato-substituted alkyl, thiocarboxylato-substituted alkyl, (amino-substituted acyl)alkyl, heterocyclic, carboxylic acid ester, w-cyanoalkyl, w-carboxylic ester-alkyl, halo C₁₋₇ alkyl, C₂₋₇ alkenyl, C₂₋₇ alkynyl, arylalkenyl, aryloxyalkyl, arylalkyl and aryl, wherein the aryl moiety of each of said arylalkenyl, aryloxyalkyl, arylalkyl and aryl radicals is optionally substituted with one or more substituents independently selected from the group consisting of halogen, C₁₋₇ alkyl, C₂₋₇ alkenyl, C₂₋₇ alkynyl, halo C₁₋₇ alkyl, nitro, hydroxyl, sulfhydryl, amino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thio-heterocyclic, arylalkylthio, heterocyclic-substituted alkylthio, formyl, carbamoyl, thiocarbamoyl, ureido, thioureido, sulfonamido, hydroxylamino, alkoxy-amino, mercaptoamino, thioalkylamino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazino, alkylhydrazino and phenylhydrazino;
R₃ and R₄ are independently selected from the group consisting of hydrogen, heteroaryl and aryl groups, wherein said heteroaryl or aryl groups are optionally substituted with one or more substituents selected from the group consisting of halogen, C₁₋₇ alkyl, C₂₋₇ alkenyl, C₂₋₇ alkynyl, halo C₁₋₇ alkyl, nitro, hydroxyl, sulfhydryl, amino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thio-heterocyclic, arylalkylthio, heterocyclic-substituted alkylthio, formyl, carbamoyl, thiocarbamoyl, ureido, thioureido, sulfonamido, hydroxylamino, alkoxy-amino, mercaptoamino, thioalkylamino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazino, alkylhydrazino and phenylhydrazino, provided that R₃ and R₄ are not both hydrogen, and further provided that R₄ is hydrogen when R₂ is monoarylamino,
or a pharmaceutical acceptable addition salt or a stereochemical isomeric form thereof
or a N-oxide thereof or a solvate thereof.

12. A pharmaceutical composition according to claim 11, further comprising one or more biologically-active drugs being selected from the group consisting of immunosuppressant and/or immunomodulator drugs, antineoplastic drugs, phosphodiesterase-4 inhibitors and antiviral agents.

13. A method of treatment of a disease mediated by phosphodiesterase-4 activity in a patient, comprising the administration of an effective amount, preferably a phosphodiesterase-4 inhibiting amount, of a pyrido(3,2-d)pyrimidine derivative.

14. A method of treatment according to claim 13, wherein said pyrido(3,2-d)pyrimidine derivative has the general formula: 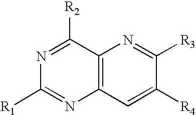
wherein:
R₁ is selected from the group consisting of hydrogen, halogen, cyano, carboxylic acid, acyl, thioacyl, alkoxycarbonyl, acyloxy, carbonate, carbamate, C₁₋₇ alkyl, aryl, amino, acetamido, N-protected amino, (mono- or di) C₁₋₇ alkylamino, (mono- or di) arylamino, (mono- or di) C₃₋₁₀ cycloalkylamino, (mono- or di) hydroxy C₁₋₇ alkylamino, (mono- or di) C₁₋₄ alkyl-arylamino, mercapto C₁₋₇ alkyl, C₁₋₇ alkyloxy, and groups of the formula R₆—NR₇R₁₂, wherein R₆ is a bond or C₁₋₃ alkylene, wherein R₇ and R₁₂ are independently selected from the group consisting of hydrogen, C₁₋₇ alkyl, C₂₋₇ alkenyl, C₂₋₇ alkynyl, aryl, arylalkyl, C₃₋₁₀ cycloalkyl and heteroaryl, or wherein R₇ and R₁₂ together form a heterocycle,
R₂ is selected from the group consisting of (mono- or di-) C₁₋₁₂ alkylamino; monoarylamino; diarylamino; (mono- or di-) C₃₋₁₀ cycloalkylamino; (mono- or di-) hydroxyC₁₋₇ alkylamino; (mono- or di-) C₁₋₄ alkylarylamino; (mono- or di-) arylC₁₋₄ alkylamino; morpholinyl; mercapto C₁₋₇ alkyl; C₁₋₇ alkoxy, homopiperazinyl and piperazinyl, wherein said homopiperazinyl or piperazinyl is optionally N-substituted with a substituent R₅ selected from the group consisting of formyl, acyl, thioacyl, amide, thioamide, sulfonyl, sulfinyl, carboxylate, thiocarboxylate, amino-substituted acyl, alkoxyalkyl, C₃₋₁₀ cycloalkyl-alkyl, C₃₋₁₀ cycloalkyl, dialkylaminoalkyl, heterocyclic-substituted alkyl, acyl-substituted alkyl, thioacyl-substituted alkyl, amido-substituted alkyl, thioamido-substituted alkyl, carboxylato-substituted alkyl, thiocarboxylato-substituted alkyl, (amino-substituted acyl)alkyl, heterocyclic, carboxylic acid ester, ω-cyanoalkyl, ω-carboxylic ester-alkyl, halo C₁₋₇ alkyl, C₂₋₇ alkenyl, C₂₋₇ alkynyl, arylalkenyl, aryloxyalkyl, arylalkyl and aryl, wherein the aryl moiety of each of said arylalkenyl, aryloxyalkyl, arylalkyl and aryl radicals is optionally substituted with one or more substituents independently selected from the group consisting of halogen, C₁₋₇ alkyl, C₂₋₇ alkenyl, C₂₋₇ alkynyl, halo C₁₋₇ alkyl, nitro, hydroxyl, sulfhydryl, amino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thio-heterocyclic, arylalkylthio, heterocyclic-substituted alkylthio, formyl, carbamoyl, thiocarbamoyl, ureido, thioureido, sulfonamido, hydroxylamino, alkoxy-amino, mercaptoamino, thioalkylamino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazino, alkylhydrazino and phenylhydrazino;
R₃ and R₄ are independently selected from the group consisting of hydrogen halogen, heteroaryl and aryl groups, wherein said heteroaryl or aryl groups are optionally substituted with one or more substituents selected from the group consisting of halogen, C₁₋₇ alkyl, C₂₋₇ alkenyl, C₂₋₇ alkynyl, halo C₁₋₇ alkyl, nitro, hydroxyl, sulfhydryl, amino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thio-heterocyclic, arylalkylthio, heterocyclic-substituted alkylthio, formyl, carbamoyl, thiocarbamoyl, ureido, thioureido, sulfonamido, hydroxylamino, alkoxy-amino, mercaptoamino, thioalkylamino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazino, alkylhydrazino and phenylhydrazino, provided that R₃ and R₄ are not both hydrogen,
or a pharmaceutical acceptable addition salt or a stereochemical isomeric form thereof
or a N-oxide thereof or a solvate thereof.

15. A method of treatment according to claim 13, wherein said disease is erectile dysfunction.

16. A method of treatment according to claim 13, wherein said administration is transurethral administration.

17. A pyrido(3,2-d)pyrimidine derivative represented by the structural formula (II) 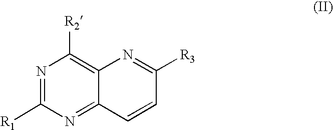
or the structural formula (III) 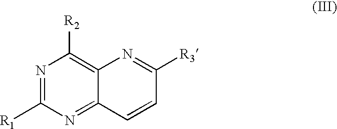
or the structural formula (IV) 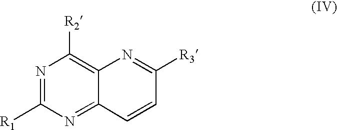
wherein:
R₁ is selected from the group consisting of hydrogen, halogen, cyano, carboxylic acid, acyl, thioacyl, alkoxycarbonyl, acyloxy, carbonate, carbamate, C₁₋₇ alkyl, aryl, amino, acetamido, N-protected amino, (mono- or di) C₁₋₇ alkylamino, (mono- or di) arylamino, (mono- or di) C₃₋₁₀ cycloalkylamino, (mono- or di) hydroxy C₁₋₇ alkylamino, (mono- or di) C₁₋₄ alkyl-arylamino, mercapto C₁₋₇ alkyl, C₁₋₇ alkyloxy, and groups of the formula R₆—NR₇R₁₂, wherein R₆ is a bond or C₁₋₃ alkylene, wherein R₇ and R₁₂ are independently selected from the group consisting of hydrogen, C₁₋₇ alkyl, C₂₋₇ alkenyl, C₂₋₇ alkynyl, aryl, arylalkyl, C₃₋₁₀ cycloalkyl and heteroaryl, or wherein R₇ and R₁₂ together form a heterocycle;
R₂ is selected from the group consisting of (mono- or di-) C₁₋₁₂ alkylamino; monoarylamino; diarylamino; (mono- or di-) C₃₋₁₀ cycloalkylamino; (mono- or di-) hydroxyC₁₋₇ alkylamino; (mono- or di-) C₁₋₄ alkylarylamino; (mono- or di-) arylC₁₋₄ alkylamino; morpholinyl; mercapto C₁₋₇ alkyl; C₁₋₇ alkoxy, homopiperazinyl and piperazinyl, wherein said homopiperazinyl or piperazinyl is optionally N-substituted with a substituent R₅
R₅ is selected from the group consisting of formyl, acyl, thioacyl, amide, thioamide, sulfonyl, sulfinyl, carboxylate, thiocarboxylate, amino-substituted acyl, alkoxyalkyl, C₃₋₁₀ cycloalkyl-alkyl, C₃₋₁₀ cycloalkyl, dialkylaminoalkyl, heterocyclic-substituted alkyl, acyl-substituted alkyl, thioacyl-substituted alkyl, amido-substituted alkyl, thioamido-substituted alkyl, carboxylato-substituted alkyl, thiocarboxylato-substituted alkyl, (amino-substituted acyl)alkyl, heterocyclic, carboxylic acid ester, ω-cyanoalkyl, ω-carboxylic ester-alkyl, halo C₁₋₇ alkyl, C₂₋₇ alkenyl, C₂₋₇ alkynyl, arylalkenyl, aryloxyalkyl, arylalkyl and aryl, wherein the aryl moiety of each of said arylalkenyl, aryloxyalkyl, arylalkyl and aryl radicals is optionally substituted with one or more substituents independently selected from the group consisting of halogen, C₁₋₇ alkyl, C₂₋₇ alkenyl, C₂₋₇ alkynyl, halo C₁₋₇ alkyl, nitro, hydroxyl, sulfhydryl, amino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thio-heterocyclic, arylalkylthio, heterocyclic-substituted alkylthio, formyl, carbamoyl, thiocarbamoyl, ureido, thioureido, sulfonamido, hydroxylamino, alkoxy-amino, mercaptoamino, thioalkylamino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazino, alkylhydrazino and phenylhydrazino;
R₃ is independently selected from the group consisting of hydrogen, heteroaryl and aryl groups, wherein said heteroaryl or aryl groups are optionally substituted with one or more substituents selected from the group consisting of halogen, C₁₋₇ alkyl, C₂₋₇ alkenyl, C₂₋₇ alkynyl, halo C₁₋₇ alkyl, nitro, hydroxyl, sulfhydryl, amino, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thio-heterocyclic, arylalkylthio, heterocyclic-substituted alkylthio, formyl, carbamoyl, thiocarbamoyl, ureido, thioureido, sulfonamido, hydroxylamino, alkoxy-amino, mercaptoamino, thioalkylamino, acylamino, thioacylamino, cyano, carboxylic acid or esters or thioesters or halides or anhydrides or amides thereof, thiocarboxylic acid or esters or thioesters or halides or anhydrides or amides thereof, alkylamino, cycloalkylamino, alkenylamino, cyclo-alkenylamino, alkynylamino, arylamino, arylalkylamino, hydroxyalkylamino, mercaptoalkylamino, heterocyclic amino, hydrazino, alkylhydrazino and phenylhydrazino;
R₂′ is selected from the group consisting of:
piperazinyl or homopiperazinyl wherein one or more carbon atoms of said piperazinyl or homopiperazinyl are independently substituted with C₁₋₄ alkyl, or two carbon atoms of said piperazinyl or homopiperazinyl together with their alkyl substituent form a C₁₋₄ alkylene group, and wherein said piperazinyl or homopiperazinyl is optionally N-substituted with R₅;
piperidin-4-yl-amino, piperidin-3-yl-amino, piperidin-4-yl-oxy, pyrrolidin-1-yl, 3-amino-pyrrolidin-1-yl, pyrrolidin-3-yl-amino or 2,6-diazabicyclo[3.2.0]heptan-2-yl, wherein said piperidin-4-yl, piperidin-3-yl, 3-amino-pyrrolidin-1-yl, pyrrolin-3-yl or 2,6-diazabicyclo[3.2.0]heptan-2-yl is optionally N-substituted with R₅ or C₁₋₄alkylarylcarbamoyl; or wherein any carbon atom of said piperidin-1-yl may be further substituted with one or more substituent selected from the group consisting of hydrocy, aryl, C₁₋₄alkylcarbamoyl, C₁₋₄alkoxycarbonyl and C₁₋₄alkylarylcarbamoyl;
piperazinyl or homopiperazinyl being N-substituted with a substituent selected from the group consisting of C₁₋₄alkyl; arylcarbamoyl-substituted alkanoyl; arylalkanoyl wherein alkanoyl is substituted with one or more substituents selected from the group consisting of amino, hydroxy and halogen; mono-C₁₋₄alkylaryl-carbamoyl; di-C₁₋₄alkylaryl-carbamoyl; tri-C₁₋₄alkylaryl-carbamoyl; mono-C₁₋₄alkylaryl-C₁₋₄alkylcarbamoyl; di-C₁₋₄alkylaryl-C₁₋₄alkylcarbamoyl; tri-C₁₋₄alkylaryl-C₁₋₄alkylcarbamoyl; alkoxycarbonyl; alkanoyl substituted with one or more substituents independently selected from the group consisting of amino, alkoxycarbonyl, alkylcarbamate, arylamido and arylcarbamoyl; arylalkanoyl substituted by alkylcarbamate; cycloalkylcarbamoyl; alkoxyalkanoyl; dialkyl-carbamoyl; heterocyclic carbamoyl C₁₋₄alkyl; arylC₁₋₄alkylcarbamoyl; heterocyclic carbonyl C₁₋₄alkyl and aryl C₁₋₄alkylcarbamoyl C₁₋₄alkyl;
triazolyl; heterocyclic amino; heterocyclic C₁₋₄alkylamino; alkoxy C₁₋₄alkylamino; amino cycloalkylamino; amino C₂₋₁₄alkylamino; amino C₁₋₆alkylamino wherein the N-atom is further substituted with C₁₋₄alkylarylcarbamoyl or aryloxy C₁₋₄alkanoyl; aryl C₁₋₄alkoxy; 3-amino-pyrrolidin-1-yl; N—C₁₋₄alkyl-N-arylcarbamoyl; and; C₁₋₄alkyl or alkanoyl substituted heterocyclic carbonyl C₁₋₄alkylamino; and,
R₃′ is an aryl group substituted with one or more substituents selected from the group consisting of heterocyclic; C₃₋₁₀ cycloalkylcarbamoyl; C₁₋₄ alkylcarbamoyl; C₁₋₄ alkylsulfonyl; C₁₋₄ alkylsulfonamido; C₁₋₄ alkyl-carboxylate; C₁₋₄ alkyl and C₁₋₄ alkoxy substituted with one or more substituents selected from the group consisting of amino, halogen, cyano and C₁₋₄alkoxy;
or a pharmaceutical acceptable addition salt or a stereochemical isomeric form thereof
or a N-oxide thereof or a solvate thereof.

18. A pyrido(3,2-d)pyrimidine derivative according to claim 17, being selected from the group consisting of
2-amino-4-[4-(4-chlorophenylcarbamoyl-acetyl)piperazin-1-yl]-6-(4-fluorophenyl)-pyrido[3,2-d]pyrimidine
(S)-2-amino-4-[4-(4-chlorophenoxyacetyl)-2-methylpiperazin-1-yl]-6-(4-fluorophenyl)-pyrido[3,2-d]pyrimidine
(S)-2-amino-4-{4-[3-(4-chlorophenyl)-2-aminopropionyl]piperazin-1-yl}-6-(4-fluorophenyl)-pyrido[3,2-d]pyrimidine
(R)-2-amino-4-(4-benzoyl-2-methylpiperazin-1-yl)-6-(4-fluorophenyl)-pyrido[3,2-d]pyrimidine
(S)-4-[2-amino-6-(4-fluorophenyl)-pyrido[3,2-d]pyrimidin-4-yl]-3-methylpiperazine-1-carboxylic acid benzyl ester
(S,S)-5-[2-amino-6-(4-fluorophenyl)-pyrido[3,2-d]pyrimidin-4-yl]-2,5-diazabicyclo[2.2.1]heptane-2-carboxylic acid tert-butyl ester
4-[(3-methylphenyl carbamoyl)-piperazin-1-yl]-6-(4-fluorophenyl)pyrido[3,2-d]pyrimidine
4-[(3-methylphenyl carbamoyl)-piperazin-1-yl]-6-(3-fluoro-4-ethoxyphenyl)pyrido[3,2-d]pyrimidine
4-[(3-methylphenyl carbamoyl)-piperazin-1-yl]-6-(3-chloro-4-ethoxyphenyl)pyrido[3,2-d]pyrimidine
4-[(3-methylphenyl carbamoyl)-piperazin-1-yl]-6-(3,4,5-trifluorophenyl)pyrido[3,2-d]pyrimidine
4-[(3-methylphenyl carbamoyl)-piperazin-1-yl]-6-(2-thienyl)pyrido[3,2-d]pyrimidine
4-[(3-methylphenyl carbamoyl)-piperazin-1-yl]-6-(2-furanyl)pyrido[3,2-d]pyrimidine
2-(N-methoxyethylamino)methyl-4-[N-(3-methylphenylcarbamoyl)piperazin-1-yl]-6-(4-fluorophenyl)-pyrido[3,2-d]pyrimidine
2-amino-4-[N-(4-chlorophenoxyacetyl)-piperazin-1-yl]-6-(4-cyanomethylphenyl)-pyrido(3,2-d)pyrimidine
2-amino-4-[N-(4-chlorophenoxyacetyl)-piperazin-1-yl]-6-(3-methoxymethylphenyl)-pyrido(3,2-d)pyrimidine
2-amino-4-[N-(4-chlorophenoxyacetyl)-piperazin-1-yl]-6-(4-cyanomethoxyphenyl)-pyrido(3,2-d)pyrimidine
2-amino-4-[N-(4-chlorophenoxyacetyl)-piperazin-1-yl]-6-(4-acetoxyphenyl)-pyrido(3,2-d)pyrimidine
2-amino-4-[N-(4-chlorophenoxyacetyl)-piperazin-1-yl]-6-(3-methoxy-4-acetoxy-phenyl)-pyrido(3,2-d)pyrimidine
2-amino-4-(4-Hydroxy-piperidine-1-carboxylic acid m-tolylamide)-6-(4-fluorophenyl)-pyrido(3,2-d)-pyrimidine
2-amino-4-{4-[N-(tert-butoxycarbonyl)-glycyl]-piperazin-1-yl}-6-(3,4-di-methoxyphenyl)-pyrido[3,2-d]pyrimidine
2-amino-4-[4-(4-chlorophenoxyacetyl)piperazin-1-yl]-6-(4-(methanesulfonyl)-phenyl)pyrido-[3,2-d]pyrimidine
2-amino-4-{4-[N-(tert-butoxycarbonyl)-glycyl]-piperazin-1-yl}-6-(4-fluorophenyl)-pyrido[3,2-d]pyrimidine
2-amino-4-[4-(4-chlorophenoxyacetyl)piperazin-1-yl]-6-[N-3-(methanesulfonamidophenyl)]-pyrido-[3,2-d]pyrimidine
2-amino-4-[4-(2-hydroxyacetyl)piperazin-1-yl]-6-(4-fluorophenyl)-pyrido[3,2-d]pyrimidine
2-amino-4-[4-(4-chlorophenoxyacetyl)piperazin-1-yl]-6-(4-trifluoromethoxyphenyl)-pyrido-[3,2-d]pyrimidine
2-amino-4-[4-(4-chlorophenoxyacetyl)piperazin-1-yl]-6-[4-(hydroxymethyl)phenyl]pyrido-[3,2-d]pyrimidine
2-amino-4-[4-(4-chlorophenoxyacetyl)piperazin-1-yl]-6-[4-(methylaminocarbonyl)phenyl]-pyrido-[3,2-d]pyrimidine
2-amino-4-[4-(aminoacetyl)-piperazin-1-yl]-6-(4-fluorophenyl)-pyrido[3,2-d]pyrimidine
2-amino-4-[4-(4-chlorophenoxyacetyl)piperazin-1-yl]-6-[4-(aminomethyl)phenyl]pyrido-[3,2-d]pyrimidine
2-amino-4-[4-(4-chlorophenoxyacetyl)piperazin-1-yl]-6-[4-(cyclopropylaminocarbonyl)phenyl]-pyrido-[3,2-d]pyrimidine
2-amino-4-{4-[N-(1-adamantylcarbamoyl)]-piperazin-1-yl}-6-(4-fluorophenyl)-pyrido[3,2-d]pyrimidine
2-amino-4-[4-(N-cyclopentylcarbamoyl)-piperazin-1-yl]-6-(4-fluorophenyl)-pyrido[3,2-d]pyrimidine
2-amino-4-[4-(methoxyacetyl)piperazin-1-yl]-6-(4-fluorophenyl)-pyrido[3,2-d]pyrimidine
2-amino-4-[1-(tert-butoxycarbonyl)piperid-3-ylamino]-6-(4-fluorophenyl)-pyrido[3,2-d]pyrimidine
2-amino-4-[4-(2-chloropropionyl)piperazin-1-yl]-6-(4-fluorophenyl)-pyrido[3,2-d]pyrimidine
2-amino-4-[(R)-3-Boc-amino-pyrrolidin-1-yl]-6-(4-fluorophenyl)-pyrido(3,2-d)pyrimidine
2-amino-4-[4-(diethylcarbamyl)piperazin-1-yl]-6-(4-fluorophenyl)-pyrido[3,2-d]pyrimidine
2-amino-4-[4-(dimethylcarbamyl)piperazin-1-yl]-6-(4-fluorophenyl)-pyrido[3,2-d]pyrimidine
2-amino-4-[4-(diisopropylcarbamyl)piperazin-1-yl]-6-(4-fluorophenyl)-pyrido[3,2-d]pyrimidine
2-amino-4-[(S)-3-Boc-amino-pyrrolidin-1-yl]-6-(4-fluorophenyl)-pyrido(3,2-d)pyrimidine
2-amino-4-[1-(tert-butoxycarbonyl)piperid-4-ylamino]-6-(4-fluorophenyl)-pyrido[3,2-d]pyrimidine
2-amino-4-[4-(methoxycarbonylacetyl)piperazin-1-yl]-6-(4-fluorophenyl)-pyrido[3,2-d]pyrimidine
2-amino-4-[3-(R)-3-methyl phenyl carbamoyl pyrrolidin-1-yl]-pyrido(3,2-d)pyrimidine
2-amino-4-[3-(S)-3-methyl phenyl carbamoyl pyrrolidin-1-yl]-pyrido(3,2-d)pyrimidine
2-amino-4-[(S)-3-methyl-phenyl carbamoyl amino-pyrrolidin-1-yl]-6-(4-fluorophenyl)-pyrido(3,2-d)pyrimidine
N-(2-{-[2-amino-6-(4-fluorophenyl)-pyrido[3,2-d]pyrimidin-4-yl]-piperazin-1-yl}-2-oxoethyl)-4-chlorobenzamide
(S)-[2-{4-[2-amino-6-(4-fluorophenyl)-pyrido[3,2-d]pyrimidin-4-yl]-piperazin-1-yl}-1-(4-chlorobenzyl)-2-oxoethyl]-carbamic acid tert-butyl ester
N-(2-{4-[2-amino-6-(4-fluorophenyl)-pyrido[3,2-d]pyrimidin-4-yl]-piperazin-1-yl}-2-oxoethyl)benzamide
2-amino-4-[1-(4-chlorophenoxyacetyl)piperid-4-ylamino]-6-(4-fluorophenyl)-pyrido[3,2-d]pyrimidine
(S,S)-1-{5-[2-amino-6-(4-fluorophenyl)-pyrido[3,2-d]pyrimidin-4-yl]-2,5-diazabicyclo[2.2.1]hept-2-yl}-2-(4-chlorophenoxy)-ethanone,
2-amino-4-[1-(phenoxyacetyl)piperid-4-ylamino]-6-(4-fluorophenyl)-pyrido[3,2-d]pyrimidine,
4-[6-(4-Fluoro-phenyl)-2-imidazol-1-ylmethyl-pyrido[3,2-d]pyrimidin-4-yl]-piperazine-1-carboxylic acid m-tolylamide,
4-[6-(4-Fluoro-phenyl)-2-[1,2,4]triazol-1-ylmethyl-pyrido[3,2-d]pyrimidin-4-yl]-piperazine-1-carboxylic acid m-tolylamide,
4-[6-(4-Fluoro-phenyl)-2-pyrrolidin-1-ylmethyl-pyrido[3,2-d]pyrimidin-4-yl]-piperazine-1-carboxylic acid m-tolylamide,
4-[6-(4-Fluoro-phenyl)-2-[1,2,3]triazol-1-ylmethyl-pyrido[3,2-d]pyrimidin-4-yl]-piperazine-1-carboxylic acid m-tolylamide,
4-[6-(4-Fluoro-phenyl)-2-pyrazol-1-ylmethyl-pyrido[3,2-d]pyrimidin-4-yl]-piperazine-1-carboxylic acid m-tolylamide,
2-(4-Chloro-phenoxy)-N-[4-{4-[2-(4-chloro-phenoxy)-acetyl]-piperazin-1-yl}-6-(4-fluoro-phenyl)-pyrido[3,2-d]pyrimidin-2-yl]-acetamide,
4-[2-(N′,N′-Dimethyl-hydrazinomethyl)-6-(4-fluoro-phenyl)-pyrido[3,2-d]pyrimidin-4-yl]-piperazine-1-carboxylic acid m-tolylamide,
4-(4-tert-Butoxycarbonyl-piperazin-1-yl)-6-(4-fluoro-phenyl)-pyrido[3,2-d]pyrimidine-2-carboxylic acid ethyl ester,
4-{4-[2-(4-Chloro-phenoxy)-acetyl]-piperazin-1-yl}-6-(4-fluoro-phenyl)-pyrido[3,2-d]pyrimidine-2-carboxylic acid ethyl ester,
4-{4-[2-(4-Chloro-phenoxy)-acetyl]-piperazin-1-yl}-6-(4-fluoro-phenyl)-pyrido[3,2-d]pyrimidine-2-carboxylic acid amide,
4-{4-[2-(4-Chloro-phenoxy)-acetyl]-piperazin-1-yl}-6-(4-fluoro-phenyl)-pyrido[3,2-d]pyrimidine-2-carboxylic acid methylamide,
4-[2-Cyanomethyl-6-(4-fluoro-phenyl)-pyrido[3,2-d]pyrimidin-4-yl]-piperazine-1-carboxylic acid m-tolylamide,
4-{4-[2-(4-Chloro-phenoxy)-acetyl]-piperazin-1-yl}-6-(4-fluoro-phenyl)-pyrido[3,2-d]pyrimidine-2-carboxylic acid propylamide,
4-{4-[2-(4-Chloro-phenoxy)-acetyl]-piperazin-1-yl}-6-(4-fluoro-phenyl)-pyrido[3,2-d]pyrimidine-2-carboxylic acid (2-methoxy-ethyl)-amide,
4-{4-[2-(4-Chloro-phenoxy)-acetyl]-piperazin-1-yl}-6-(4-fluoro-phenyl)-pyrido[3,2-d]pyrimidine-2-carboxylic acid dimethylamide,
2-(4-Chloro-phenoxy)-1-{4-[6-(4-fluoro-phenyl)-2-(pyrrolidine-1-carbonyl)-pyrido[3,2-d]pyrimidin-4-yl]-piperazin-1-yl}-ethanone,
4-{4-[2-(4-Chloro-phenoxy)-acetyl]-piperazin-1-yl}-6-(4-fluoro-phenyl)-pyrido[3,2-d]pyrimidine-2-carboxylic acid cyclopropylamide,
4-(2-{4-[2-Amino-6-(4-fluoro-phenyl)-pyrido[3,2-d]pyrimidin-4-yl]-piperazin-1-yl}-2-oxo-ethoxy)-benzoic acid methyl ester,
4-{4-[2-(4-Chloro-phenoxy)-acetyl]-piperazin-1-yl}-6-(4-fluoro-phenyl)-pyrido[3,2-d]pyrimidine-2-carboxylic acid,
4-{4-[2-(4-Chloro-phenoxy)-acetyl]-piperazin-1-yl}-6-(4-fluoro-phenyl)-pyrido[3,2-d]pyrimidine-2-carbonitrile,
4-(6-Pyridin-3-yl-pyrido[3,2-d]pyrimidin-4-yl)-piperazine-1-carboxylic acid m-tolylamide,
4-(6-Thiophen-3-yl-pyrido[3,2-d]pyrimidin-4-yl)-piperazine-1-carboxylic acid m-tolylamide,
4-[2-Amino-6-(4-fluoro-phenyl)-pyrido[3,2-d]pyrimidin-4-yl]-piperazine-1,3-dicarboxylic acid 3-tert-butylamide 1-m-tolylamide,
4-(6-Chloro-pyrido[3,2-d]pyrimidin-4-yl)-piperazine-1-carboxylic acid m-tolylamide,
4-(6-Pyridin-4-yl-pyrido[3,2-d]pyrimidin-4-yl)-piperazine-1-carboxylic acid m-tolylamide,
4-[6-(2-Chloro-phenyl)-pyrido[3,2-d]pyrimidin-4-yl]-piperazine-1-carboxylic acid m-tolylamide,
4-(6-o-Tolyl-pyrido[3,2-d]pyrimidin-4-yl)-piperazine-1-carboxylic acid m-tolylamide,
4-(6-Furan-3-yl-pyrido[3,2-d]pyrimidin-4-yl)-piperazine-1-carboxylic acid m-tolylamide,
4-[6-(2-Cyano-phenyl)-pyrido[3,2-d]pyrimidin-4-yl]-piperazine-1-carboxylic acid (4-chloro-2-trifluoromethyl-phenyl)-amide, and
4-[6-(2-Cyano-phenyl)-pyrido[3,2-d]pyrimidin-4-yl]-piperazine-1-carboxylic acid (4-tert-butyl-phenyl)-amide,
or a pharmaceutical acceptable addition salt or a stereochemical isomeric form thereof
or a N-oxide thereof or a solvate thereof.

19. A pharmaceutical composition comprising one or more pharmaceutically acceptable carriers and a pyrido(3,2-d)pyrimidine derivative according to claim 17.

20. A pharmaceutical composition comprising one or more pharmaceutically acceptable carriers and a pyrido(3,2-d)pyrimidine derivative according to claim 17 and further comprising one or more biologically-active drugs being selected from the group consisting of immunosuppressant and/or immunomodulator drugs, antineoplastic drugs, phosphodiesterase-4 inhibitors and antiviral agents.

21. A pharmaceutical composition comprising one or more pharmaceutically acceptable carriers and a pyrido(3,2-d)pyrimidine derivative according to claim 18.

22. A pharmaceutical composition comprising one or more pharmaceutically acceptable carriers and a pyrido(3,2-d)pyrimidine derivative according to claim 18 and further comprising one or more biologically-active drugs being selected from the group consisting of immunosuppressant and/or immunomodulator drugs, antineoplastic drugs, phosphodiesterase-4 inhibitors and antiviral agents.

23. A method of treatment of a disease selected from the group consisting of rheumatoid arthritis, Crohn's disease, ulcerative colitis, uveitis, multiple sclerosis, atopic dermatis, psoriasis, lupus erythematosus, or prevention of transplant rejection in a patient, comprising the administration of an effective amount, of a pyrido(3,2-d)pyrimidine derivative according to claim 17.

24. A method of treatment of a disease selected from the group consisting of rheumatoid arthritis, Crohn's disease, ulcerative colitis, uveitis, multiple sclerosis, atopic dermatis, psoriasis, lupus erythematosus, or prevention of transplant rejection in a patient, comprising the administration of an effective amount, of a pyrido(3,2-d)pyrimidine derivative according to claim 18.
